(19) United States
(12) Patent Application Publication
(10) Pub. No.: US 2026/0345183 A1
(43) Pub. Date: Nov. 5, 2026

(54) PULSE ENHANCED BALLOON CATHETER AND METHODS OF USE

(71) Applicant: CAGENT VASCULAR, INC., Wayne, PA (US)

(72) Inventors: Robert M. Giasolli, Orange, CA (US); Peter Johansson, Wayne, PA (US)

(21) Appl. No.: 19/662,961

(22) Filed: Apr. 29, 2026

Related U.S. Application Data
(60) Provisional application No. 63/797,393, filed on Apr. 30, 2025.
(60) Provisional application No. 63/946,831, filed on Dec. 22, 2025.

(51) Int. Cl. A61B 17/22; A61B 17/00; A61M 25/10

(52) U.S. Cl. CPC A61B 17/22012; A61B 17/00234; A61M 25/104; A61B 2017/00305; A61B 2017/00778; A61B 2017/22001; A61B 2017/22008; A61B 2017/22025; A61B 2017/22065; A61M 2025/1004; A61M 2025/1086; A61M 2205/3331; A61M 2210/12

(57) ABSTRACT

Systems and methods can involve medical balloons with raised features for forming serrations within plaque for angioplasty, lithotripsy and/or atherectomy. The intravascular systems can include a balloon and a plurality of raised features. The intravascular systems can include a generator configured to generate a change in pressure, wherein the pressure change directly correlates with a change in diameter and length of the balloon. The intravascular systems can include a generator configured to generate a pressure wave. Methods can include generating a change in pressure, wherein the pressure change directly correlates with a change in diameter and length of the balloon. Methods can include generating a pressure wave.

### CROSS-REFERENCE TO RELATED APPLICATIONS

This application claims priority benefit to U.S. Provisional Patent Application No. 63/797,393, filed Apr. 30, 2025 and U.S. Provisional Patent Application No. 63/946,831, filed Dec. 22, 2025, the entirety of which is hereby incorporated by reference herein.

### BACKGROUND

### Field

Certain embodiments disclosed herein relate generally to one or more serrated structures adhered to or integrated with a medical balloon, such as an angioplasty balloon, and systems and methods of use for breaking up calcium, calcium deposits, or plaque, at or below the surface or within the vessel. Additional use of wave mechanics applied by a generator to the serrated medical balloon can decrease the time required to breakup calcium, calcium deposits, or plaque, at or below the surface or within the vessel.

### Description of the Related Art

Human vessel diseases, such as atherosclerotic occlusive disease that is the primary cause of stroke, heart attack, limb loss, and death in the United States and the industrialized world, are a significant contributor to organ failure. For instance, atherosclerotic plaque forms a hard layer along the wall of a blood vessel and is comprised of calcium, cholesterol, compacted thrombus, and cellular debris. As the atherosclerotic disease progresses, the blood supply intended to pass through the blood vessel is diminished or even prevented by the occlusive process. One of the most widely utilized methods of treating clinically significant human vessel diseases, including atherosclerotic plaque, is balloon angioplasty.

Balloon angioplasty is a method of opening blocked or narrowed vessels in the body. The balloon angioplasty catheter is placed into the diseased vessel (such as an artery) from a remote access site that is created either percutaneously or through open exposure of the artery. The catheter must navigate a variety of anatomical pathways and move along the inside of the diseased vessel over a wire that guides the way of the catheter. The portion of the catheter with the balloon attached is placed at the location of the disease (e.g., atherosclerotic plaque) that requires treatment. The balloon is generally inflated to a size that is consistent with, or slightly larger than, the reference vessel diameter of the artery (a neighboring artery region prior to developing occlusive disease).

Due to the nature of occlusive diseases the progression of the disease moves from minimally stenotic (a small amount of the artery lumen is reduced) to a totally closed or occluded (chronic total occlusion). Independent of the disease state, when the balloon is inflated, the plaque is stretched, compressed, fractured, or broken, depending on its composition, location, and the amount of pressure exerted by the balloon. The plaque is heterogeneous and may be soft in some areas or hard in others causing unpredictable cleavage planes to form under standard balloon angioplasty. Balloon angioplasty can cause plaque disruption and sometimes even arterial injury at the angioplasty site.

### SUMMARY

Arterial disease is inherently heterogeneous. Micro-structures within the cellular matrix induce stress concentrations at specific microscopic sites. When these stress concentrations exceed a critical threshold, they cause localized micro-damage within the micro-structure. This critical threshold can be interpreted as weaknesses within the cellular network and are therefore sites for fracture initiation event. Plain balloon angioplasty is intended to accelerate stress concentration and induce a fracture event. In doing so balloon angioplasty increases the micro-damage produced at the cellular level during balloon inflation. The inclusion of periodic linear raised features on the outside of a balloon can induce an intentional lower stress state thereby introducing a purposeful linear micro cracking pathway or fracturing along the periodically linear raised features producing a serrated line. The raised features create initiation sites as tiny prick points. This new induced micro-cracking pathway reduces internal stress in the affected areas. With serration technology, the pressure of the balloon can cause cleavage planes to connect the initiation sites along longitudinal cracks along the axis of the vessel.

With the addition of pulse wave technologies, there can be synergistic results including that the initiation sites more effectively and rapidly release the stored elastic potential energy in the form of pressure waves. The pressure waves can be mechanical vibrations that can propagate through the vessel including calcified regions that originate from disturbances such as the initiation sites. The pressure waves can cause particles to oscillate. The pressure waves can be mechanical waves that travel through material. The pressure waves can be a subset of elastic waves wherein particles move parallel to the wave direction. The pressure waves can compress the medium. These pressure waves can manifest as spontaneous ultrasonic signals originating from the initiation sites or stress concentration sites. The use of ultrasonic frequency pressure waves inclusive with the pulsed wave balloon angioplasty further can enhance the cellular break down of hardened calcified tissue. The use of ultrasonic frequency pressure waves inclusive with serration balloon angioplasty can significantly enhance the cellular break down of hardened calcified tissue. As the raised features of the serration angioplasty balloon initiate micro-cracking, the micro-cracking reduces internal stress in the affected areas, releasing some of the stored elastic potential energy as pressure waves. These pressure waves manifest as spontaneous ultrasonic signals originating from the impact zones at the raised features, such as the outwardly facing surfaces on the strip tips, that are in contact with the hardened calcium.

In some embodiments, an intravascular system can be provided. The intravascular system can include a balloon. The intravascular system can include a plurality of raised features. The intravascular system can include a generator configured to generate a pressure wave. The intravascular system can include a pulsatile pump system configured to generate a pressure wave. The intravascular system can include a pump system configured to generate an ultrashort pulse wave. The intravascular systems pulse wave can be a hydraulic pulse that includes an ultrasonic frequency wave. In some intravascular systems the pulse wave can be generated by an extracorporeal generator. The intravascular systems extracorporeal generator can include elements of the pulse wave that are configured to generate a change in pressure in the system. In some embodiments, the pressure change directly correlates with a change in diameter and length of the balloon. The intravascular system can include a generator configured to generate a high frequency pulse wave. The intravascular system can include a generator configured to generate a single high frequency pulse wave. The intravascular system can include a generator configured to generate a series of high frequency pulse waves. In some embodiments, the periodicity of the series of high frequency pulse waves can be set by the operator. In some embodiments, the periodicity of the series of high frequency pulse waves can be varied from 0.5 Hz to 20 Hz, while in some designs the pulse wave can be set between 1 Hz and 10 Hz. While other designs allow the operator to set the frequency of the pulses between 1 Hz and 3 Hz. In some embodiments, the intensity of the pulse wave directly correlates with the length of the catheter. In some embodiments, the intensity of the wave directly correlates with a change in diameter and length of the balloon. In some embodiments, the intensity of the wave can be altered independent of the diameter and length of the balloon. In some embodiments, the intensity of the wave can be altered independent of the catheter length. In some embodiments, the intensity of the wave can be altered by the operator. There can be a correlation between the intensity of the wave and the sound pressure spectrum level. In some embodiments, the sound pressure spectrum level can be varied from 0.1 dB re microPa\hat{ }2/Hz to 1000 dB re microPa\hat{ }2/Hz, but in some designs the sound pressure spectrum level can be set between 10 dB re microPa\hat{ }2/Hz to 500 dB re microPa\hat{ }2/Hz. The design can allow the operator to set the sound pressure spectrum level of the pulses between 10 dB re microPa\hat{ }2/Hz to 100 dB re microPa\hat{ }2/Hz.

In some embodiments, the generator can be configured to increase pressure in the balloon to expand the balloon. In some embodiments, the generator can be configured to decrease pressure in the balloon to deflate the balloon. In some embodiments, the generator can be configured to cycle between balloon growth and retraction. In some embodiments, the generator can be configured to move the plurality of raised features in and out of tissue. In some embodiments, the generator can be configured to move the plurality of raised features in and out of tissue at a dimension equal to the change in balloon radius. In some embodiments, an extracorporeal generator produces a high frequency wave that includes frequencies in the ultrasonic band from outside the body and transmits the energy pulse through the catheter to the balloon. In some embodiments, the extracorporeal generator produces a pressure wave as part of the component of the high frequency wave that includes frequencies in the ultrasonic band. In some embodiments, the generator can be configured to produce a lithotripsy effect. The lithotripsy effect can be independently associated with the ultrasonic frequency emanating from the balloon. The lithotripsy effect can be independently associated with the features on the balloon. The lithotripsy effect can be magnified or amplified at the raised features on the balloon. The lithotripsy effect can be independently associated with the balloon growth and retraction due to the pressure wave. The lithotripsy effect can be a combination of one or more of the effects produced by the generator. In some embodiments, the generator can be configured to break calcium bonds. In some embodiments, the generator can be configured to cyclically displace an incompressible fluid. In some embodiments, the generator can be configured to deliver a waveform of energy. In some embodiments, the generator can be configured to induce a range of acoustic frequencies caried within a wave. In some embodiments, the generator can be configured to induce primarily a single defined frequency within a wave. In some embodiments the length of the catheter can be longer than the resonant frequency being produced at the generator. In some embodiments, the primary frequency can be in the ultrasonic frequency range. In some embodiments, the primary frequency can be acoustic but not ultrasonic. In some embodiments, the primary frequency includes ultrasonic and sonic frequencies. In some embodiments, the ultrasound produces one or more single crest followed by a gap in time with no wave. In some embodiments, the ultrasound periodicity between one or more single crest followed by a gap in time with no wave can be set by the operator. In some embodiments, the plurality of raised features comprise metal. In some embodiments, the plurality of raised features can be disposed on an outer surface of the balloon. In some embodiments, the plurality of raised features can be configured to facilitate translation of pulse energy through mechanical friction. In some embodiments, the plurality of raised features can be configured to facilitate translation of pulse energy through a mechanical interface to reduce impedance loss. In some embodiments, the plurality of raised features can be configured to facilitate translation of the frequency. In some embodiments, the plurality of raised features can be configured to amplify the effect of translation of the frequency into the tissue. In some embodiments, the plurality of raised features can be configured to facilitate a mechanical interface. In some embodiments, the plurality of raised features can be configured to facilitate a reduction in impedance loss.

In some embodiments, a method can be provided. The method can include positioning a catheter at a treatment site in a vessel. In some embodiments, the catheter can include a balloon and a plurality of raised features. The method can include expanding the balloon at the treatment site to form serrations. The method can include generating a change in pressure. In some embodiments, the pressure changes directly correlate with a change in diameter and length of the balloon.

In some embodiments, an acute or shock like event can be used to generate a pressure wave with a frequency in the acoustic spectrum. In some embodiments, an acute or shock like event produces a pressure wave with a frequency in the ultrasound spectrum. In some embodiments, an acute or shock like event produces a pressure wave with a frequency in both the acoustic and the ultrasound spectrums. In some embodiments, generating the change in pressure produces a wave through cavitation (e.g., implosion of a bubble). In some embodiments, cavitation can include the formation, growth, and collapse of a bubble. The cavitation can occur in the liquid within the vessel. The cavitation can occur when local pressure drops below the vapor pressure of the liquid. The cavitation can cause liquid jets extending from raised features of the balloon. In some embodiments, the localized change in pressure can occur outside the balloon producing a wave through cavitation (e.g., implosion of a bubble). In some embodiments, generating the change in pressure comprises transitioning between two discrete levels of pressure over time at a constant frequency. In some embodiments, generating the change in pressure comprises transitioning between more than two discrete levels of pressure over time at a constant frequency. In some embodiments, generating the change in pressure comprises transitioning between two discrete levels of pressure over time at a variable frequency. In some embodiments, generating the change in pressure comprises transitioning between more than two discrete levels of pressure over time at a variable frequency. In some embodiments, generating the change in pressure reduces the time for lesion yielding. In some embodiments, generating the change in pressure minimizes neighboring tissue damage.

In fracture mechanics, dynamic loading, wherein the load changes over time, can lead to unstable cracking and structural failure quicker than static loading conditions. The inclusion of an acoustic frequency to the dynamic load can further facilitate the failure mechanism. The inclusion of an ultrasonic frequency to the dynamic load can further facilitate the failure mechanism. The combination of longitudinally oriented raised features, with the intent to facilitate longitudinally oriented fractures in diseased vessels, along with dynamic pressure loading with acoustic and/or ultrasonic waves, can offer an innovative advantage to modifying disease and returning vessel compliance to hardened, heavily calcified vessels, such as arteries or veins. In addition, it has been observed that the use of linearly oriented serrated strips with pressure waves increases lumen gain, volumetric blood flow, and vessel compliance and decreases vessel injury and vessel recoil compared with the use of linearly oriented serrated strips without pressure waves. The inclusion of dynamic pressure loading to these systems offers additional advantages of decreasing time to yield the diseased lesion while increasing the effectiveness of increasing lumen gain, volumetric blood flow, and vessel compliance and decrease vessel injury and vessel recoil.

In some embodiments, an intravascular system is provided. The system can include a balloon configured to be filled with a liquid medium. The system can include a plurality of raised features extending from the balloon. The system can include a generator for generating a pressure wave configured to propagate though the liquid medium used to fill the balloon and break calcium deposits.

In some embodiments, the generator can include a cam hammer and a piston. In some embodiments, the generator can include a deflectable membrane. In some embodiments, the pressure wave can include a shock wave. In some embodiments, the generator can produce longitudinal cracks in the vessel, including the plaque, at or below the surface. In some embodiments, the generator can compress plaque. In some embodiments, the generator can produce a lithotripsy effect. In some embodiments, the generator can break calcium bonds. In some embodiments, the pressure wave can include a maximum pressure of 10 atm within the balloon. In some embodiments, the plurality of raised features can magnify energy delivery.

In some embodiments, a method is provided. The method can include positioning a balloon and a plurality of raised features at a treatment site. The method can include expanding the balloon at the treatment site. The method can include generating a pressure wave that propagates through a liquid medium used to fill the balloon and breaks calcium deposits.

In some embodiments, generating the pressure wave can include translating a cam hammer. In some embodiments, generating the pressure wave can include striking a piston. In some embodiments, generating the pressure wave can include deflecting a membrane. In some embodiments, generating the pressure wave can include spiking the pressure to 10 atm within the balloon. In some embodiments, generating the pressure wave can include generating a shock wave. In some embodiments, the method can include increasing the maximum pressure of the pressure wave by compressing a spring. In some embodiments, the method can include increasing the maximum pressure of the pressure wave by increasing the cam diameter. In some embodiments, the method can include increasing the pulse frequency of the pressure wave by increasing the speed of a motor. In some embodiments, generating the change in pressure can include transitioning between two discrete levels of pressure over time. In some embodiments, the pressure wave creates cracks in plaque. In some embodiments, the pressure wave creates cracks in vessel plaque. In some embodiments, the pressure wave creates cracks in a vessel inclusive of plaque. In some embodiments, the pressure wave creates cracks in a vessel including the plaque, at or below the surface.

In some embodiments, a system is provided. The system can include a balloon configured to be expanded with a liquid medium. The system can include a plurality of strips arranged along a surface of the balloon. In some embodiments, the plurality of strips can include raised features. The system can include a generator operably coupled to the balloon and configured to percussively move a deflectable membrane to produce a pressure wave with ultrasonic frequency. In some embodiments, the pressure wave can propagate through the liquid medium. In some embodiments, the generator can include a synchronous motor.

In some embodiments, the synchronous motor uses a direct current electric power supply. In some embodiments, the synchronous motor is configured to be driven at speeds under 5 Hz. In some embodiments, the synchronous motor is configured to driven at speeds of 1 Hz, 2 Hz, or 3 Hz. In some embodiments, the generator controls frequency of pressure wave generation by controlling a speed of the synchronous motor. In some embodiments, a speed of the motor is configured to be adjusted by a user via a speed control. In some embodiments, the generator further comprises an adjuster configured to change a position of the synchronous motor. In some embodiments, the system can include a handle operably coupled to the balloon, wherein the motor is within the handle. In some embodiments, the motor is configured to translate relative to the handle.

In some embodiments, a system is provided. The system can include a balloon configured to be expanded with a liquid medium. The system can include a plurality of strips arranged along a surface of the balloon. In some embodiments, the plurality of strips can include raised features. The system can include a generator operably coupled to the balloon and configured to percussively move a deflectable membrane to produce a pressure wave with ultrasonic frequency. In some embodiments, the pressure wave can propagate through the liquid medium. In some embodiments, the generator can include a cam hammer and a spring. In some embodiments, an amplitude of the pressure wave can be adjustable by adjusting a compression of the spring.

In some embodiments, the generator can include an adjuster. In some embodiments, the generator can be configured to limit the amplitude of the pressure wave by creating stops for movement of the adjuster. In some embodiments, the system can include a handle operably coupled to the balloon. In some embodiments, the cam hammer and the spring can be located within the handle. In some embodiments, the generator can include a motor comprising a motor shaft. In some embodiments, the spring can be expanded and compressed as the motor shaft of the motor rotates. In some embodiments, the generator can include a piston, wherein the cam hammer applies an impact force to the piston based on a compression of the spring. In some embodiments, the impact force applied to the piston by the cam hammer can increase as compression of the spring increases. In some embodiments, the cam hammer can be moved by a force of the spring. In some embodiments, the generator can be configured to periodically raise a pressure inside the balloon to about 10 atm.

In some embodiments, a system is provided. The system can include a catheter. The system can include a balloon coupled to the catheter and configured to be expanded with a liquid medium. The system can include a plurality of strips arranged along a surface of the balloon. In some embodiments, the plurality of strips can include raised features.

The system can include a handle coupled to the catheter and housing a generator configured to percussively move a deflectable membrane to produce a pressure wave with ultrasonic frequency. In some embodiments, the pressure wave can propagate through the liquid medium. In some embodiments, each of a frequency and an amplitude of the pressure wave can be adjustable via the handle. In some embodiments, the frequency of the pressure wave can be 1 Hz, 2 Hz, or 3 Hz. In some embodiments, the generator can include a speed control. In some embodiments, the frequency of the pressure wave is configured to be adjusted via the speed control. In some embodiments, the generator further comprises a motor. In some embodiments, the frequency of the pressure wave is adjustable by adjusting a speed of a motor. In some embodiments, the generator can include a spring. In some embodiments, the amplitude of the pressure wave can be adjustable by compressing the spring. In some embodiments, the pressure waves can be delivered continuously when the generator is powered on. In some embodiments, the generator can include an adjuster. In some embodiments, the amplitude of the pressure wave can be adjusted by rotating the adjuster. In some embodiments, the generator can include a motor. In some embodiments, the amplitude of the pressure wave can be adjusted by translation of the motor. In some embodiments, the pressure wave can include a peak positive pressure between 8 atm and 20 atm. In some embodiments, the pressure wave can include a pulse duration between 1 microsecond and 5 milliseconds. In some embodiments, the pressure wave can include a rise time or time delay per unit of pressure of between 1 nanosecond per pascal and 500 nanoseconds per pascal. In some embodiments, the pressure wave can include a pulse width as defined as the full width at half maximum (FWHM) of between 0.5 microsecond and 3 milliseconds.

In some embodiments, a method is provided. The method can include providing a balloon and a plurality of strips arranged along a surface of the balloon. In some embodiments, the plurality of strips can include raised features. The method can include positioning the balloon at a treatment site. The method can include expanding the balloon with a liquid medium at the treatment site. The method can include generating a pressure wave that propagates though the liquid medium. In some embodiments, the raised features of the plurality of strips undulate with the pressure wave.

In some embodiments, the raised features of the plurality of strips can undulate at the speed of sound. In some embodiments, the raised features of the plurality of strips can undulate along a length of the balloon as the pressure wave propagates along the length of the balloon. In some embodiments, each strip of the plurality of strips can be unconnected to other strips of the plurality of strips. In some embodiments, a portion of the raised features of the plurality of strips can move into engagement with plaque while a portion of raised features of the plurality of strips are not in engagement with plaque. In some embodiments, each strip of the plurality of strips can be longitudinally orientated along the surface of the balloon. In some embodiments, the pressure wave can apply a load along a length of each strip as the wave propagates distally. In some embodiments, the raised features of the plurality of strips can undulate by rising proximal edges of the plurality of strips followed by rising the first proximal raised features, and then neighboring raised features. In some embodiments, the raised features of the plurality of strips can rise to the same height during undulation. In some embodiments, undulation of the raised features of the plurality of strips can accelerate formation of microperforations. In some embodiments, undulation of the raised features of the plurality of strips can accelerate the formation of cracks.

In some embodiments, a method is provided. The method can include providing a balloon and a plurality of strips arranged along a surface of the balloon. In some embodiments, the plurality of strips can include raised features. The method can include positioning the balloon at a treatment site. The method can include expanding the balloon with a liquid medium at the treatment site. The method can include generating a pressure wave that propagates though the liquid medium. In some embodiments, the raised features of the plurality of strips zipper into plaque.

In some embodiments, the raised features of the plurality of strips zipper out of the plaque. In some embodiments, the plurality of strips is spaced apart circumferentially. In some embodiments, the raised features of the plurality of strips zipper into the plaque due to the elongate nature of the plurality of strips. In some embodiments, the raised features of the plurality of strips zipper into the plaque due to the column strength of the plurality of strips. In some embodiments, the raised features of the plurality of strips zipper into the plaque due to bending direction of the plurality of strips. In some embodiments, the raised features of the plurality of strips move with a rolling motion. In some embodiments, the raised features along a strip of the plurality of strips do not move simultaneously. In some embodiments, the pressure wave transmits longitudinally down a body of the balloon.

In some embodiments, a method is provided. The method can include providing a balloon and a plurality of strips arranged along a surface of the balloon. In some embodiments, the plurality of strips comprises raised features. In some embodiments, positioning the balloon at a treatment site. In some embodiments, the method can include expanding the balloon with a liquid medium at the treatment site. In some embodiments, the method can include generating a pressure wave that propagates though the liquid medium, wherein the raised features of the plurality of strips move in a wave.

In some embodiments, the pressure wave travels along a length of the balloon from a proximal end of the balloon to a distal end of the balloon. In some embodiments, the raised features of the plurality of strips progressively penetrate into diseased tissue. In some embodiments, a point source proximal to the balloon transmits the pressure wave. In some embodiments, each strip of the plurality of strips is angularly spaced along the surface of the balloon. In some embodiments, the raised features along a strip of the plurality of strips do not move simultaneously. In some embodiments, progressive movement of each raised feature in series along each strip facilitates linear vessel expansion at the speed of the pressure wave.

In some embodiments, a system is provided. The system can include a balloon configured to be expanded with a liquid medium. The system can include a strip comprising raised features. In some embodiments, the strip can be arranged along a surface of the balloon. The system can include a generator to produce a pressure wave with ultrasonic frequency. In some embodiments, the pressure wave can propagate though the liquid medium. In some embodiments, the pressure wave can propagate along a length of the balloon from a proximal end to the distal end. In some embodiments, the raised features can amplify the pressure wave to accelerate a speed at which a fracture occurs while maintaining a propensity for the fracture to occur in a longitudinally oriented line.

In some embodiments, the raised features can amplify the pressure wave by at least 50×. In some embodiments, the raised features can amplify the pressure wave by at least 100×. In some embodiments, the pressure wave can travel longitudinally along an edge of a raised feature. In some embodiments, the raised features can include sloped sidewalls that direct the pressure wave. In some embodiments, the generator can be proximal to the balloon. In some embodiments, the strip can be longitudinally oriented on the surface of the balloon. In some embodiments, the length of the balloon can be between 10 mm and 200 mm. In some embodiments, the length of the balloon can be between 40 mm and 120 mm. The balloon 100 can be longer than other therapeutic balloons. The balloon can be 50% longer than other angioplasty balloons. The balloon can be 100% longer than other intravascular lithotripsy angioplasty balloons. The longer balloon can lead to faster treatment times. In some embodiments, a diameter of the balloon can be between 1.5 mm and 12 mm.

In some embodiments, a system is provided. The system can include a balloon configured to be expanded with a liquid medium. The system can include a strip comprising raised features. In some embodiments, the strip can be arranged along a surface of the balloon. The system can include a generator configured to produce a pressure wave with ultrasonic frequency. In some embodiments, the pressure wave can propagate though the liquid medium to impact each raised feature against plaque in series. In some embodiments, the pressure of the entire pressure wave can be magnified to a single raised feature.

In some embodiments, the raised features can magnify the pressure between 1000× and 40,000×. In some embodiments, the pressure wave can travel longitudinally as directed by a long edge of an outward surface of the raised features. In some embodiments, the generator can be external to the balloon. In some embodiments, the strip and the raised features can include a material that is more reflective than the balloon.

In some embodiments, a system is provided. The system can include a balloon configured to be expanded with a liquid medium. The system can include a strip comprising raised features. In some embodiments, the strip is arranged along a surface of the balloon. The system can include a generator configured to produce a pressure wave with ultrasonic frequency. In some embodiments, the pressure wave can propagate though the liquid medium. In some embodiments, the pressure wave can be transmitted along a length of the balloon. In some embodiments, a magnifying effect of each raised feature can be driven by the motion of the pressure wave through the balloon.

In some embodiments, the magnifying effect can be between 50× and 1500×. In some embodiments, wherein the magnifying effect can be greater than 100×. In some embodiments, a transverse component and a longitudinal component of the pressure wave can be magnified by the raised features. In some embodiments, the length of the balloon can be between 10 mm and 200 mm.

In some embodiments, a system is provided. The system can include a balloon. The system can include a plurality of strips comprising raised features arranged along a surface of the balloon. The system can include a handle comprising a reservoir, a piston, and a membrane. In some embodiments, the membrane can transfer energy from the piston to fluid inside the reservoir to generate an ultrasonic wave therein.

In some embodiments, the system is a modular, hand-held medical instrument. In some embodiments, the system can supply energy at a frequency of 1 Hz, 2 Hz, or 3 Hz. In some embodiments, the system can break down calcifications in tissue. In some embodiments, the piston can deflect the membrane to transfer energy to the fluid. In some embodiments, the force applied to the piston by the cam hammer can increase as the spring is compressed.

In some embodiments, a system is provided. The system can include a balloon. The system can include a plurality of strips comprising raised features arranged along a surface of the balloon. The system can include a proximal handle comprising a reservoir, a piston, and a membrane. In some embodiments, the membrane can be deflected by the piston thereby causing a volumetric change in the reservoir and creating an ultrasonic wave in fluid in the reservoir.

In some embodiments, the reservoir can be transparent. In some embodiments, the system can include a pressure sensor. In some embodiments, the pressure sensor is located within the reservoir. In some embodiments, the balloon can be in fluid communication with the reservoir. In some embodiments, the volumetric change in the reservoir can expand the balloon. In some embodiments, the ultrasonic wave can effect a body of the balloon progressively from a proximal end to a distal end of the balloon. In some embodiments, the membrane can include flexible material. In some embodiments, the membrane can be concave. In some embodiments, the volumetric change can expand the balloon within the upper and lower boundaries of the compliance curve of the balloon. In some embodiments, the ultrasonic wave can accelerate as the ultrasonic wave travels from the reservoir into a catheter.

In some embodiments, a system is provided. The system can include a balloon configured to be expanded with a liquid medium. The system can include a plurality of strips comprising raised features arranged along a surface of the balloon. The system can include a proximal handle comprising a reservoir and a membrane configured to be deflected thereby causing an increase in pressure in the reservoir and creating an ultrasonic wave in liquid medium in the reservoir.

In some embodiments, the system can include pressure of the reservoir is monitored by the pressure sensor. In some embodiments, the membrane can include silicone. In some embodiments, the membrane can be deflected to induce a pressure change from 2 atm to 12 atm in the balloon. In some embodiments, a peak positive pressure of the ultrasonic wave can be between 8 atm and 20 atm. In some embodiments, the pressure of the balloon can be consistent along a length of the balloon. In some embodiments, the ultrasonic wave can move through each progressive medium delivering a proportional component of the wave energy relative to the reflection coefficient of each medium the ultrasonic wave passes through. In some embodiments, the reservoir can include a funnel shape. In some embodiments, the membrane can be deflected to match the growth of the balloon at both the upper and lower boundaries of pressure corresponding to the compliance curves of the balloon.

In some embodiments, a method is provided. The method can include providing a balloon and a plurality of strips arranged along a surface of the balloon. In some embodiments, the plurality of strips can include raised features. The method can include positioning the balloon within a vessel. The method can include sequentially inflating and deflating the balloon to allow the balloon to inch past an occlusion.

In some embodiments, the raised features move with an up and down motion. In some embodiments, the raised features do not move simultaneously. In some embodiments, a portion of the raised features of the plurality of strips move into engagement with the occlusion while a portion of raised features of the plurality of strips are not in engagement with the occlusion. In some embodiments, each strip of the plurality of strips is spaced apart. In some embodiments, the method can include creating a pressure wave for lithotripsy.

In some embodiments, a method is provided. The method can include providing a balloon and a plurality of strips arranged along a surface of the balloon. In some embodiments, the plurality of strips can include raised features. The method can include positioning the balloon within a vessel. The method can include inflating the balloon and deflating the balloon to reach a treatment site.

In some embodiments, the raised features have a rising and falling form. In some embodiments, the plurality of strips allows bending in certain directions. In some embodiments, the plurality of strips allows a rolling motion under the influence of a pressure wave. In some embodiments, the method can include inflating the balloon at the treatment site to form serrations. In some embodiments, the method can include generating a change in pressure, wherein the change in pressure directly correlates with a change in diameter and length of the balloon. In some embodiments, the balloon is taken to the treatment site across a calcified lesion. In some embodiments, the raised features are blunt. In some embodiments, the method can include breaking calcium bonds. In some embodiments, the method can include modifying calcified plaque.

In some embodiments, a method is provided. The method can include providing a balloon and a plurality of strips arranged along a surface of the balloon. In some embodiments, the plurality of strips can include raised features. . . . The method can include positioning the balloon within a vessel. The method can include navigating the vessel by inflating and deflating the balloon to reach a treatment site.

In some embodiments, the raised features have a rolling motion. In some embodiments, a pressure wave introduces bending to the plurality of strips. In some embodiments, the method can include creating serrations, indentations, and/or microperforations at the treatment site. In some embodiments, the balloon navigates across narrow or occluded areas in the vessel. In some embodiments, the method can include creating ultrasonic waves at the treatment site. In some embodiments, the raised features are wedge shaped. In some embodiments, the method improves crossability through narrow lesions, tortious paths, and/or hardened disease. In some embodiments, the method can include providing lithotripsy along an entire length of the balloon. In some embodiments, the method can include providing lithotripsy along an entire circumference of the balloon.

In some embodiments, a system is provided. The system can include a balloon. The system can include a strip comprising raised features. In some embodiments, the raised features can have a greater length than width. In some embodiments, a longitudinal axis of the strip can be longitudinally oriented along the balloon. The system can include a generator configured to propagate a wave through the balloon. In some embodiments, the raised features can follow the wave that propagates through the balloon. In some embodiments, the generator can produce a series of waves. In some embodiments, the series of waves can repeatably drive the raised features in a tapping motion.

In some embodiments, the tapping motion can reduce a time required to yield a hardened diseased tissue. In some embodiments, the tapping motion can accelerate creation of microperforations in plaque. In some embodiments, the tapping motion can accelerate creation of a crack along a cleavage plane. In some embodiments, the tapping motion can reduce a time required to create cracks between microperforations. In some embodiments, the tapping motion can reduce a time required to break calcium bonds. In some embodiments, the system can return to a baseline pressure between each wave of the series of waves. In some embodiments, the wave can include an ultrasonic pressure wave. In some embodiments, the series of waves can repeatably drive lobes of the balloon in a tapping motion.

In some embodiments, a system is provided. The system can include a balloon. The system can include a strip comprising raised features. In some embodiments, the raised features can have a greater length than width. In some embodiments, a longitudinal axis of the strip can be longitudinally oriented along the balloon. The system can include a generator that can produce a series of pressure waves. In some embodiments, all of the raised features along a length of the balloon can move radially outward with the rise in pressure with the balloon. In some embodiments, all of the raised features along the length of the balloon can move radially inward with the fall in pressure with the balloon.

In some embodiments, the proximal raised features can move radially outward before middle raised features. In some embodiments, the middle raised features can move radially outward before distal raised features. In some embodiments, all of the raised features can make contact with a vessel wall simultaneously. In some embodiments, the system can utilize a hammering motion. In some embodiments, a frequency of the hammer motion can be independent of the speed of the pressure wave. In some embodiments, the pressure wave can propagate through the balloon to create short bursts of pressure or shock waves including ultrasonic frequencies that enter surrounding tissue. In some embodiments, the system can generate a serrated line in the tissue in a shorter period of time than when the series of pressure waves is not used. In some embodiments, each pressure wave in the series of pressure waves can spike to the same maximum pressure. In some embodiments, a maximum pressure of any pressure wave in the series of pressure waves can be between 8 atm and 20 atm. In some embodiments, any two pressure waves of the series of pressure waves can include a pressure difference between 4 atm and 20 atm. In some embodiments, the pressure waves can propagate from a source proximal to the proximal end of the balloon. In some embodiments, the raised features can create cavitation bubbles outside of the balloon.

In some embodiments, a system is provided. The system can include a balloon. The system can include a strip comprising raised features. In some embodiments, the raised features can have a greater length than width. In some embodiments, a longitudinal axis of the strip can be longitudinally oriented along the balloon. The system can include a generator configured to create a series of impacts between the raised features and diseased tissue via pressure waves delivered to the balloon to accelerate fracturing with each consecutive pressure wave.

In some embodiments, the pressure waves can propagate from outside of a body of the balloon. In some embodiments, the pressure waves can propagate from a pressure wave source within a proximal handle of the system. In some embodiments, the pressure wave can transmit a frequency in the ultrasound spectrum. In some embodiments, the raised features can repeatedly impact without longitudinal movement of the balloon. In some embodiments, the raised features can create cavitation bubbles. In some embodiments, the raised features can accelerate fracturing through the internal elastic lamina and into a calcium sheet of the diseased tissue. In some embodiments, the strip and the raised features amplify the mechanical force of the pressure wave by 50 times

In some embodiments, a system is provided. The system can include a balloon. The system can include a plurality of strips comprising raised features arranged along a surface of the balloon. The system can include a piston and a membrane. In some embodiments, the membrane is configured to be deflected by the piston thereby creating a pressure wave.

In some embodiments, a dwell time of the piston against the membrane is altered by a pulse frequency. In some embodiments, a dwell time of the piston against the membrane is altered by a speed of a motor. In some embodiments, a dwell time of the piston against the membrane is altered by a diameter of a cam can. In some embodiments, a dwell time of the piston against the membrane is altered by a slope length of a cam can insert. In some embodiments, a dwell time of the piston against the membrane is altered by an arc shape of a cam can insert. In some embodiments, a dwell time of the piston against the membrane is between 55 milliseconds and 65 milliseconds at 3 Hz pulse frequency. In some embodiments, a dwell time of the piston against the membrane is between 65 milliseconds and 75 milliseconds at 2 Hz pulse frequency. In some embodiments, a dwell time of the piston against the membrane is between 80 milliseconds and 90 milliseconds at 1 Hz pulse frequency.

In some embodiments, a method is provided. The method can include providing a balloon and a plurality of strips arranged along a surface of the balloon. In some embodiments, the plurality of strips comprises raised features. The method can include positioning the balloon at a treatment site. The method can include expanding the balloon with a liquid medium at the treatment site. The method can include generating a pressure wave that causes cavitation.

In some embodiments, the cavitation occurs in the liquid within the vessel. In some embodiments, the cavitation causes liquid jets extending from the raised features. In some embodiments, the cavitation is confined into a crack under the raised features. In some embodiments, the cavitation is caused by movement of the raised features.

In some embodiments, an intravascular system is provided. The intravascular system can include a balloon. The intravascular system can include a plurality of raised features. The intravascular system can include a pulsatile pump system configured to generate a change in pressure. In some embodiments, the pressure change directly correlates with a change in diameter and length of the balloon.

In some embodiments, the pulsatile pump system is configured to increase pressure in the balloon to expand the balloon. In some embodiments, the pulsatile pump system is configured to decrease pressure in the balloon to deflate the balloon. In some embodiments, the pulsatile pump system is configured to cycle between balloon growth and retraction. In some embodiments, the pulsatile pump system is configured to move the plurality of raised features in and out of tissue. In some embodiments, the pulsatile pump system is configured to move the plurality of raised features in and out of tissue at a dimension equal to the change in balloon radius. In some embodiments, the pulsatile pump system is configured to produce a lithotripsy effect. In some embodiments, the pulsatile pump system is configured to break calcium bonds. In some embodiments, the pulsatile pump system is configured to cyclically displace an incompressible fluid. In some embodiments, the pulsatile pump system is configured to deliver a wave form of energy. In some embodiments, the pulsatile pump system is configured to induce a defined frequency within a wave. In some embodiments, the frequency is ultrasonic. In some embodiments, the ultrasound produces one or more single crest followed by a gap in time with no wave. In some embodiments, the plurality of raised features comprise metal. In some embodiments, the plurality of raised features are disposed on an outer surface of the balloon. In some embodiments, the plurality of raised features are configured to facilitate translation of pulse energy through mechanical friction. In some embodiments, the plurality of raised features are configured to facilitate translation of pulse energy through a mechanical interface to reduce impedance loss.

In some embodiments, a method is provided. The method can include positioning a catheter at a treatment site in a vessel, the catheter comprising a balloon and a plurality of raised features. The method can include expanding the balloon at the treatment site to form serrations. The method can include generating a change in pressure. In some embodiments, the pressure change directly correlates with a change in diameter and length of the balloon.

In some embodiments, generating the change in pressure produces a wave in the infrasound spectrum. In some embodiments, generating the change in pressure produces a wave in the ultrasound spectrum. In some embodiments, generating the change in pressure produces a wave through cavitation of a bubble. In some embodiments, generating the change in pressure comprises transitioning between two discrete levels of pressure over time at a constant frequency. In some embodiments, generating the change in pressure comprises transitioning between more than two discrete levels of pressure over time at a constant frequency. In some embodiments, generating the change in pressure comprises transitioning between two discrete levels of pressure over time at a variable frequency. In some embodiments, generating the change in pressure comprises transitioning between more than two discrete levels of pressure over time at a variable frequency. In some embodiments, generating the change in pressure reduces the time for lesion yielding. In some embodiments, generating the change in pressure minimizes neighboring tissue damage.

In some embodiments, an intravascular system is provided. The intravascular system can include a balloon configured to be filled with a liquid medium. The intravascular system can include a plurality of raised features extending from the balloon. The intravascular system can include an assembly for generating a pressure wave configured to propagate though the liquid medium used to the fill the balloon and break calcium deposits.

In some embodiments, the assembly comprises a cam hammer and a piston. In some embodiments, the assembly comprises a deflectable membrane. In some embodiments, the pressure wave comprises a shock wave. In some embodiments, the assembly is configured to produce longitudinal cracks in plaque adhered to a vessel wall. In some embodiments, the assembly is configured to compress plaque. In some embodiments, the assembly is configured to produce a lithotripsy effect. In some embodiments, the assembly is configured to break calcium bonds. In some embodiments, the pressure wave comprises a maximum pressure of 10 atm within the balloon. In some embodiments, the plurality of raised features magnify energy delivery.

In some embodiments, a method is provided. The method can include positioning a balloon and a plurality of raised features at a treatment site. The method can include expanding the balloon at the treatment site. The method can include generating a pressure wave that propagates though the liquid medium used to the fill the balloon and breaks calcium deposits.

In some embodiments, generating the pressure wave comprises translating a cam hammer. In some embodiments, generating the pressure wave comprises striking a piston. In some embodiments, generating the pressure wave comprises deflecting a membrane. In some embodiments, generating the pressure wave comprises spiking the pressure to 10 atm within the balloon. In some embodiments, generating the pressure wave comprises generating a shock wave. In some embodiments, the method can include increasing the maximum pressure of the pressure wave by compressing a spring. In some embodiments, the method can include increasing the frequency of the pressure wave by increasing the speed of a motor. In some embodiments, generating the change in pressure comprises transitioning between two discrete levels of pressure over time. In some embodiments, the pressure wave creates longitudinal cracks in plaque adhered to a vessel wall.

### BRIEF DESCRIPTION OF THE DRAWINGS

These and other features, aspects and advantages are described below with reference to the drawings, which are intended to illustrate but not to limit the invention. In the drawings, like reference characters denote corresponding features consistently throughout similar embodiments.

FIG. 1 is a view of a pulse enhanced balloon catheter system.

FIG. 2 represents the arterial pulse waveform.

FIGS. 3 and 4 are block diagrams that schematically illustrate an exemplary system for components that might work together to produce a pulse enhanced balloon system.

FIG. 5 is a schematic view of a pump.

FIG. 6 illustrates a pulse enhanced balloon catheter system.

FIG. 7 illustrates an exploded view of the pulse enhanced balloon catheter system of FIG. 6.

FIGS. 8A-80 illustrate components of the pulse enhanced balloon catheter system of FIG. 6.

FIG. 9 illustrates a cross-sectional view of the pulse enhanced balloon catheter system of FIG. 6.

FIGS. 10-11 illustrate the pulse enhanced balloon catheter system of FIG. 6.

FIG. 12 illustrates a serration enhanced balloon catheter.

FIG. 13 is a close-up detail view of an embodiment of a raised feature on its associated strip.

FIG. 14 illustrates a schematic perspective view of various dimensions and terminology of a wedge dissector, according to some embodiments.

FIGS. 15-21 illustrate various embodiments of raised feature geometries.

FIGS. 22A-22F illustrate respective end and isometric views of various raised feature geometries, according to some embodiments.

FIGS. 23A-23D illustrate respective end and isometric views of various asymmetric raised feature geometries, according to some embodiments.

Not to be limited by theory, FIGS. 23E, 23F, and 23G show potential mechanisms of actions of a serration device.

FIG. 24 illustrates an embodiment illustrating how the unbounded surface may have a varying height, according to some embodiments.

FIGS. 25A-25K illustrate various embodiments of strips with reliefs in various locations.

FIGS. 26A-26B illustrates an embodiment of a strip with raised features where the raised feature has a sloped non-linear edges.

FIG. 27 illustrates the top of the raised feature can have a variety of the features on the radially outward facing surface that contacts the tissue.

FIG. 28 is another design illustrating an alternate variation of the serrated edge of the wedge dissector, where the central segment can include a small depression as shown.

FIG. 29 illustrates the raised features having rounded double-hump like contacting surfaces at the tip that can provide effective tissue penetration.

FIG. 30 illustrates variations on a design that provides a relatively sharp, pointed double contacting surface at the tip of each raised feature providing effective tissue penetration.

FIG. 31 illustrates a similar design that provides a relatively sharp, pointed double contacting surface at the tip of each raised feature which provides effective tissue penetration, that abut a central deeper, and more shallow valley/depression respectively.

FIGS. 32A-32K illustrate various views of one embodiment of an overall system for producing serratoplasty showing a series of serrating or scoring raised features on the outer diameter of the catheter attached to a catheter with a guidewire hub and balloon inflation hub.

FIG. 33 illustrates an electrical diagram.

FIG. 34 illustrates the frequency signal from the pressure wave.

FIG. 35 illustrates the time domain of the pressure wave.

FIGS. 36-42 are graphs of the pressure wave and diameter displacement of the balloon.

FIGS. 43-50 are graphs of the pressure input and diameter displacement of balloon.

FIGS. 51-52 illustrate the pulse enhanced balloon catheter system of FIG. 6.

FIG. 53 illustrates the pulse enhanced balloon catheter system of FIG. 6.

FIG. 54 illustrates time data collected to crack a calcium modeled ring for a catheter system without a wave generator.

FIG. 55 illustrates time data collected to crack a calcium modeled ring for a catheter system with a wave generator.

FIG. 56 illustrates the reduction in time with pressure wave generation.

FIG. 57 illustrates an upper limit boundary data for a catheter system with a wave generator.

FIG. 58A-58E is a series of illustrations showing the mechanism for the raised features to turn from a tangential orientation to being uncovered and oriented perpendicularly and subsequent penetration by the raised features.

FIG. 59 illustrates crack propagation.

FIGS. 60A-60C illustrate views of the vessel.

FIG. 61 illustrates the pulse enhanced balloon catheter system of FIG. 6 with a carrier.

FIG. 62 illustrates a graph of frequency over time.

FIG. 63 illustrates an example of energy delivery.

FIG. 64 illustrates an example of a balloon.

FIGS. 65A-65D illustrate an example of a strip with raised features.

FIGS. 66A-66B illustrate graphs of fracture incidence.

FIG. 67 illustrates images of fractures.

FIG. 68 illustrates a frequency range.

### DETAILED DESCRIPTION

Systems and methods can include a balloon having a plurality of strips disposed on an outer surface of the balloon, wherein each strip has a plurality of raised features. Systems and methods can include a generator to produce a pressure wave. The pressure waves can produce distinct effects inside of the balloon and outside of the balloon. Inside the balloon, the pressure wave can create a fluid wave. Systems and methods can produce a fluid wave that raises the raised features of the strips. The fluid wave can cause alternating regions of high pressure and low pressure that can cause corresponding movement of raised features along the strip. The fluid wave can create longitudinal motion of fluid particles of the liquid medium used to fill the balloon. The fluid wave can create regions of compression and expansion of the fluid particles in the balloon. Systems and methods are described herein that move the raised features radially, proximally to distally in a wave. Systems and methods are described herein which can produce a fluid wave capable of and effective at micro fracturing the calcium. The fluid wave can have an additive effect independent of the strips 200 and the raised features 300 which create serrations. Systems and methods are described herein wherein the fluid wave can have an additive effect independent of the pressure within the balloon 200. In some embodiments the pressure wave can be a fluid pressure wave. Outside of the balloon, the pressure wave can carry an acoustic wave when it propagates through the solid medium of the balloon 100, the strips 200, and the raised features 300. The acoustic wave can be detected at various points outside of the balloon, including at the wall of the balloon, at the raised features, and within the tissue. The acoustic wave can be in the ultrasonic frequency. Systems and methods are described herein that produce an acoustic wave comprised of a plurality of frequencies including at least one ultrasonic frequency. Systems and methods are described herein that propagate the acoustic wave through a wall of the balloon. Systems and methods are described herein that propagate the acoustic wave to break calcium, such as hardened calcified plaque in arteries or veins. Systems and methods are described herein which can produce an acoustic wave capable of and effective at micro fracturing the calcium. Systems and methods are described herein which can produce an acoustic wave capable of and effective at generating nodes of weakness in the calcium sheet. The calcium can be fractured with the acoustic waves. The acoustic wave can have an additive lithotripsy effect independent of the strips 200 and the raised features 300 which create serrations. The acoustic wave can travel through the wall of the balloon 100, the strip 200, and/or the raised features 300 to fracture calcium. This effect of the acoustic wave itself can impart forces on the calcium which is independent of the serration effect created by the raised features 300. The acoustic wave traveling through the wall of the balloon 100 can have a lithotripsy effect. The acoustic wave traveling through both the balloon 100 and the strips 200 with the raised features 300 can have a lithotripsy effect. The lithotripsy effect of the acoustic wave, in combination with the serration effect created by the raised features 300, can increase the effectiveness of the procedure, such as more effective lumen gain, quicker time to fracture, and lower pressure to fracture. Systems and methods are described herein wherein the acoustic wave can have an additive lithotripsy effect independent of the pressure within the balloon 200. The acoustic wave can be produced at any time during the procedure when fluid is within the balloon 100. The acoustic wave can be produced when the balloon 100 is at a base pressure, such as a low pressure about 1 ATM to 2 ATM. The pressure wave detected outside of the balloon 100 with the strips 200 and the raised features 300 can have various frequencies. In some embodiments, the pressure wave detected outside of the balloon 100 can be an infrasonic pressure wave having frequencies below 20 Hz. In some embodiments, the pressure wave detected outside of the balloon 100 can be an acoustic pressure wave having frequencies within the range of 20 Hz to 20 kHz. In some embodiments, the pressure wave detected outside of the balloon 100 can be an ultrasonic pressure wave having frequencies above 20 kHz. In some embodiments, the pressure wave detected outside of the balloon 100 can be an ultrasonic pressure wave having frequencies up to 200 KHz.

Systems and methods are described herein related to serration enhanced and pulse enhanced balloon catheter embodiments. Systems and methods can include synergies between serration technology and pulse wave technology to remodel the vessel. Systems and methods can include crack propagation through lines of weakness. Systems and methods are described herein that fracture calcium bonds. Systems and methods can include disrupting calcium bonds through an acoustic wave that travels from the balloon and into tissue. Systems and methods can include a generator to create pressure wave through the body of the balloon. The pressure waves can be pulsed with rapid increases and decreases in pressure. The pressure waves can have a rhythmic repetition. The pressure waves can have a number of pulses per second measured in Hz. The pressure wave can have a pulse frequency of 1 Hz, 2 Hz, 3 Hz, 4 Hz, or 5 Hz, or ranges thereof. The pressure waves can have a rate at which pressure waves are generated. Systems and methods can generate and direct pressure waves from the proximal generator to the fluid medium used to fill the balloon. Systems and methods can generate and direct pressure waves with a range of frequencies. Systems and methods can generate and direct pressure waves with a range of pressures. Systems and methods can produce specific synergies not previously known. For example, the pressure waves can accelerate the crack formation, shortening procedure time. For example, the pressure waves can be concentrated to promote linear formation of cracks for segmentation of the plaque. For example, the pressure waves can create cavitation outside of the balloon causing fracturing of calcium. For example, the pressure waves can create a shearing stress on calcium thereby reducing the thickness of the calcium bed. For example, the pressure waves can compress the calcium deposit leading to increased lumen gain. These and many other advantages are described herein. Systems and methods can produce unexpected results from the use of low frequency pulses of pressure waves. For example, the low frequency pulses of pressure waves can allow the strips to undulate. For example, the low frequency pulses of pressure waves can concentrate the pressure wave to a single raised feature 300. For example, the low frequency pulses of pressure waves can allow the strip 200 with the raised features 300 to move with the fluid wave as the fluid wave travels from the proximal end to the distal end of the balloon. For example, the low frequency pulses of pressure waves can allow cavitation to occur. These and many other advantages are described herein. Systems In some embodiments, systems and methods can generate pulses at a pulse to pulse rate under 5 Hz. Systems and methods can have the ability to maintain the pressure within the balloon below a particular threshold throughout the procedure. Systems and methods can return to a baseline pressure due to back pressure within the system. Systems and methods can increase the pressure to a peak positive pressure at the speed of sound. Systems and methods can increase the pressure to a peak positive pressure regardless of the tortuous path of the catheter. Systems and methods can combine specific ranges of paraments to produce unexpected results as described herein. For example, systems and methods can combine specific ranges of paraments such as, but not limited to, the frequency of pressure wave generation and the amplitude of pressure waves to produce the pressure waves. These parameters can be within an operating envelope of the generator. For example, the systems and methods can combine specific ranges of paraments to create a pressure wave that carries certain frequencies. The pressure waves generated with the systems and methods described herein can carry an ultrasonic frequency that breaks down lesions which narrow vessels. The pressure waves can carry an ultrasonic frequency that can fracture calcium deposits including deep and superficial deposits. The pressure waves can carry a frequency that can restore blood flow. The pressure waves can cooperate with the serrations created by the balloon 100 with the strips 200 and the raised features 300. This cooperation can produce a combined effect greater than serration alone or pressure wave generation alone. These synergies and others are described further herein. Systems and methods can both displace fluid and generate a pressure wave. Systems and methods can produce a pressure wave with minimal changes (e.g., 1% to 10%) in amplitude from the generator to the distal end of the balloon wherein the peak positive pressure remains nearly constant along the length of the balloon 100. The pressure wave can be highly stable. The system can have minimal external interference. The system can have minimal friction. The pressure wave can propagate along the length of the balloon with minimal changes in amplitude. The pressure wave can propagate along the length of the balloon with minimal changes (e.g., 1% to 10%) in amplitude from the generator due mostly to catheter dampening or attenuation leading to dB losses along the catheter length. This dampening along the catheter length can be controlled by material selection of the catheter, among other design parameters. Systems and methods can include several advantages as described in greater detail below.

Systems and methods can be serration enhanced. Systems and methods as disclosed herein can deploy the balloon with the raised features in any body lumen, including vascular lumens such as arteries and veins. The arteries could be coronary arteries, peripheral arteries, or carotid or other cerebral arteries, for example, iliac, femoral, superficial femoral, popliteal, anterior and posterior tibial, peroneal, or other peripheral vasculature, for example, left main coronary artery, right coronary artery, circumflex artery, left anterior descending, distal right coronary artery, or other coronary arteries, for example. The systems and methods may also be used in any lumen or transportation vessel found in any of the vascular, respiratory, digestive, urinary, reproductive, lymphatic, auditory, optical, or endocrine systems. The systems or methods for generating serrations in any one, two, or more of these anatomical regions, can have slightly different catheter bodies and features. In some systems and methods, the design may include catheter exchange features like those found in a monorail design while other systems may offer rapid exchange like design features. Other design features may also be included and each may take slightly different forms. Independent of the location where the systems and methods might be used, some embodiments of system and methods include raised features that may be joined together on a supporting strip, and a balloon to increase and decrease the diameter of the raised features. Together the strips with raised features and the balloon produce a serrated balloon element. The serrated balloon element can be then attached to a base catheter-like device. Systems and methods can include any balloon features or catheter features described herein.

Systems and methods can be pulse enhanced. The catheter-like device can provide pressure waves, in some embodiments, to produce a lithotripsy effect to fracture calcium. The pressure waves can be pulsed by a generator, as described herein. For instance, the pressure wave can break down the plaque. In some embodiments, the lithotripsy effect may not be limited to ultrasonic pressure waves, but rather describes the broad class of technologies that break plaque. In some embodiments, the calcium bonds of the plaque are broken by the lithotripsy effect of the pressure waves. In some embodiments, mechanical energy can produce the pressure wave delivered within a frequency range to produce the lithotripsy effect. The catheter-like device can provide pressure waves, in some embodiments, to produce an atherectomy effect to grind calcium. The inclusion of the pressure wave in the balloon, whether generated from a generator connected to the inflation hub or alternate pressure source, can produce a micro movement event at the raised features which can abrade the surface of the plaque. Systems and methods can include any pressure wave generator features described herein.

Systems and methods can include synergistic, unexpected results from combining serration enhanced and pulsed enhanced features. In some pulse enhanced serration angioplasty embodiments, the pulsatile energy can be delivered via a pressure wave, such as a variable pulsed energy wave, through the balloon body into the surrounding tissue. In some embodiments, the pulse enhanced serration angioplasty technology can include three quantifiable features that can be used together to effectuate the result. The three features can include a generator to produce pressure waves at a low frequency, e.g., generated at a frequency below 5 Hz, a serration angioplasty balloon having longitudinally oriented strips with raised features, wherein the strips are not circumferentially connected, and a source for establishing a base pressure, such as an indeflator. Combined, these features can produce three effects. In the first of the three effects, the embodiments can produce and deliver a pressure wave that can be configured to deliver energy, such as acoustic or ultrasonic energy, to the diseased tissue, for instance to fracture or break apart the disease morphology, especially calcium hardened disease. The pressure wave can be a short pulsatile energy that can be varied by both intensity of the wave and the periodicity of the wave pulse within ranges below a designated threshold. The variability of the short pulse pressure wave can be controlled by an operator, a computer, an algorithm, and/or a feedback loop, allowing for variations in the intensity and periodicity of the wave pattern to match the desired outcome during use. In the second effect, the serration balloon embodiments include linearly oriented raised features shaped to offer penetration advantage into the intimal layers of tissue. The third effect can be a synergistic effect when both the first effect, the pressure wave, and second effect, the serration balloon embodiments, are combined, and the energy is propagated through the raised features and amplified to the narrow contact zone of the raised features. . . . As the pressure wave translates through the serration balloon, the energy can be combined and amplified with mechanical energy delivery of the raised features on the balloon. The strip can undulate with the pressure wave further concentrating the energy delivery. The raised features can also produce localized cavitation events. Other synergistic effects from the combined pressure wave generation and the serrations are described herein. With these three effects integrated into a pulsed enhanced serration angioplasty system, embodiments can offer the additional advantage of fracturing diseased tissue, in the form of lithotripsy, with a high level of prediction, precision, and effectiveness. Independent of the design, the inclusion of these three effects collectively can be included in variations of pulsed enhanced serration angioplasty embodiments. The pulsatile energy in addition to the serrated strips can offer a tri-model impact on the diseased tissue.

Systems and methods can produce a serrated line. While not to be bound by a theory, the serrated line can weaken the tissue. The weakened tissue gives to the pressure of the balloon. The weakened tissue gives under the influence of time. The weakened tissue can allow the crack to propagate along the serrated line deeper until the medial layer is reached. The serrations, indentations, and/or microperforations slowly combine into a linear dissected line, and the serrated line slowly deepen through the layers of the tissue and disease. The serrations, indentations, and/or microperforations are initially formed by the strip. The linear dissected line can be formed by means of crack propagation along the series of serrations, indentations, and/or microperforations which deepen over time and as the balloon exerts more pressure. As the crack propagates, the balloon may be able to more easily push the vessel wall outward thereby deepening the serrated line. The crack propagates longitudinally between the serrations, indentations, and/or microperforations.

Systems and methods can propagate a crack. While not to be bound by a theory, the crack can deepen as the balloon expands. The crack can deepen as the pressure of the balloon 100 is increased. The crack deepens as the strip fully seats within the serrations, indentations, and/or microperforations. The crack deepens as the strip wedges more tissue apart along the height of the raised feature. The crack deepens as the balloon exerts a greater pressure of the vessel wall thereby pulling the vessel wall away from the strip. While not to be bound by a theory, the crack propagation is spaced from the unhoned tip of the raised feature. The unhoned tip of the raised feature itself doesn't not further deepen the crack. Rather, the tissue tears along the length of the raised feature to deepen the crack. The crack can be deeper than the height of the raised feature. In some methods, the cracks will trend towards alignment with the linear array of raised features 300. In some methods, due to the inhomogeneous nature of disease morphology, the crack will propagate along cellular lines of weakness. This trend for cracks to propagate along cellular weak planes can occur both in regions of raised features 300 and in regions where no raised features are present, such as the lobes.

### Systems

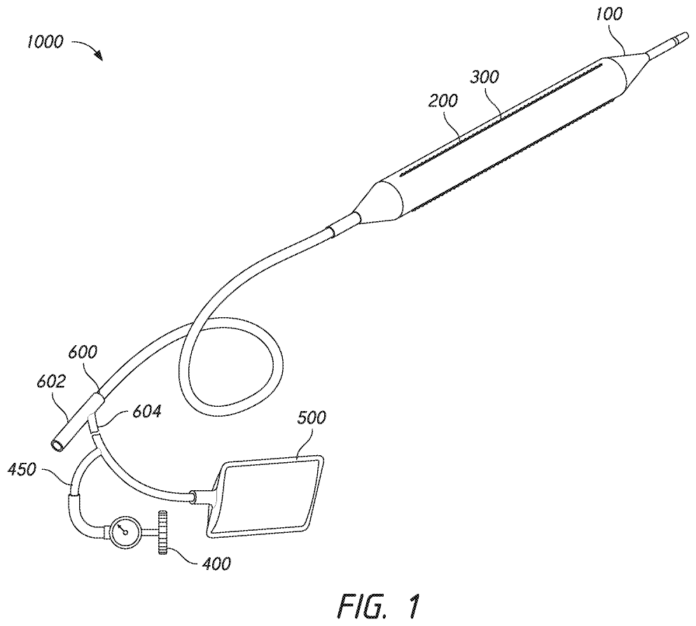

FIG. 1 illustrates a system 1000. The system 1000 can be designed for combined serration and lithotripsy as described herein. The system 1000 can include an expandable balloon 100. The expandable balloon 100 can have any feature described herein. The system 1000 can include one or more strips 200. The strip 200 can have any feature described herein. Each strip 200 can include a plurality of raised features 300 spaced apart along the surface of the strip. The raised features 300 can have any feature described herein.

The system 1000 can be considered a generator system. The system 1000 can be considered a pump system. The system 1000 can be considered a pulsatile pump system. The system 1000 can generate a change in pressure in the system. The pressure change can directly corollate with a diameter and length change in the balloon 100. The serrated strips 200 may be rigid beams of material which ride on the balloon surface. Each pulse of the system 1000 first increases pressure (phase 1) expanding the balloon 100, followed by a second decrease in pressure (phase 2) shrinking the balloon 100. Each cycle of the balloon growth and retraction moves the rigid beams of the serrated strips 200 in and out of the tissue at a dimension equal to the change in the radius of the balloon 100. This action can be produced by the pressure wave that can offer an effective lithotripsy effect capable of breaking calcium bonds.

The system 1000 can include an inflation device 400. The inflation device 400 can have any feature described herein. The inflation device 400 can be an indeflator. The inflation device 400 can deliver and regulate the fluid medium used to fill the balloon. The inflation device 400 can monitor pressure with a pressure gauge. The inflation device 400 can be an indeflator with more than a pressure gauge. The inflation device 400 can have a flow rate of up to 50 l/min. The inflation device 400 can include an integrated temperature controlling element. The inflation device 400 can be connected to the expandable balloon 100 with a connector 450. The connector 450 can be a Y-connector. The connector 450 can be a T-divider. The connector 450 can be a stopcock. The connector 450 can be a 3-way stopcock. The connector 450 can be a 4-way stopcock. The connector 450 can control the direction of liquid flow between ports. The connector 450 can include one or more luer fittings. The connector 450 can include one or more ports. The connector 450 can connect to the inflation device 400.

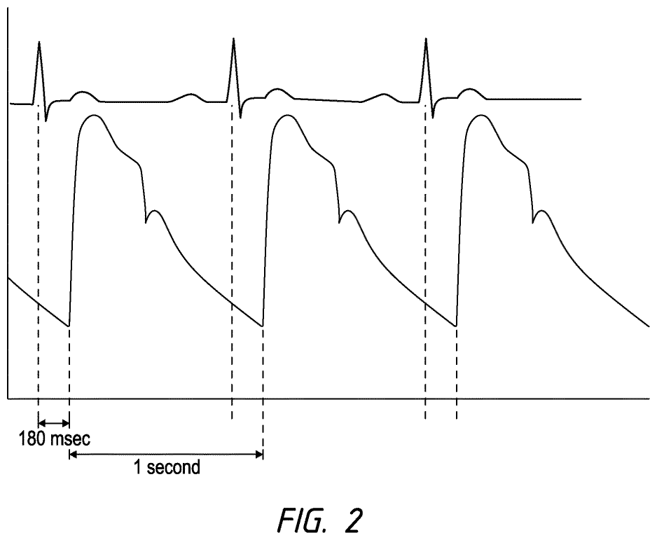

The system 1000 can include a generator 500. The generator 500 can be a pump. The generator 500 can be a pump system. The generator 500 can be a pulsatile pump system. The generator 500 can have any features of generators described herein. The generator 500 can generate pressure waves, as described herein. The generator 500 can pump with a repeatable defined regular rhythm, in some embodiments. The generator 500 can generate a waveform, in some embodiments. The generator 500 can simulate waveforms of blood in the body, in some embodiments. The generator 500 can simulate the ventricle hemodynamic waveform of the heart. The generator 500 can counter waveforms of blood in the body, in some embodiments. The generator 500 can be connected to the expandable balloon 100 with the connector 450. In some embodiments, a triggering event linked to physiological markers can be employed. For instance, intramyocardial blood pressure changes during systolic versus diastolic pressures. Therefore, the variations of cardio pressure could be interpreted through an EKG and then used to synchronize the pressure wave of the generator 500 to the appropriate known blood pressures in the myocardial vessels. In some embodiments, the waveform from the generator 500 can be configured to align with the pulsatile biological behavior of the heart. In some embodiments, the waveform is perfectly in sync with the pulsations of the heart. In some embodiments, the waveform is perfectly out of sync with the pulsations of the heart. The patient's heartbeat can be determined by an EKG or other medical technique. The patient's heart arterial pulse waveform be determined by an EKG or other medical technique. The arterial pulse waveform can represent blood pressure change within the arteries over time. FIG. 2 represents the arterial pulse waveform as an example of the waveform generated by the generator 500. The EKG which can be used to determine the waveform to be generated by the generator 500. The pulse waveform produced by the generator 500 can by synced to match the naturally produced blood pressure waveform found in the body. When the pressure wave from the generator 500 is in sync with the blood pressure waves, the additive pressure in the vessels can be magnified by combining the pressure from the generator 500 and the naturally occurring pressure. The pulse waveform produced by the generator 500 can be configured to be out of sync with the naturally produced blood pressure waveform found in the body. When the pressure wave from the generator 500 is out of sync with the blood pressure waves, the generator 500 can have a greater impacting force on the vessel as the vessel decreases in size due to the low pressure event occurring locally. In some embodiments, the pressure waves can be pulsatile. In some embodiments, the pressure waves can mimic the natural, rhythmic heartbeat. In some embodiments, the pressure waves can be nonpulsatile. In some embodiments, the pressure waves can be at pulses that do not mimic the natural, rhythmic heartbeat. The generator 500 can connect to the connector 450.

The inflation device 400 can be a stand-alone device. The inflation device 400 can be any inflation device including Merit Medical BlueDiamond™, basixTOUCH™, Boston Scientific Encore™ 26, BD Preston™, or other inflation devices. The inflation device 400 can be a custom inflation device. The inflation device 400 and the generator 500 can be integrated into one device. The inflation device 400 can incorporate the pulsatile pump function. The generator 500 can be integrated with or within the custom inflation device. The inflation device 400 and the generator 500 can be separate devices.

The inflation device 400 can be a standalone system. The generator 500 can be a standalone system. The purpose of the inflation device 400 can be to inflate the angioplasty balloon. The purpose of the inflation device 400 can be to monitor the pressure within the balloon.

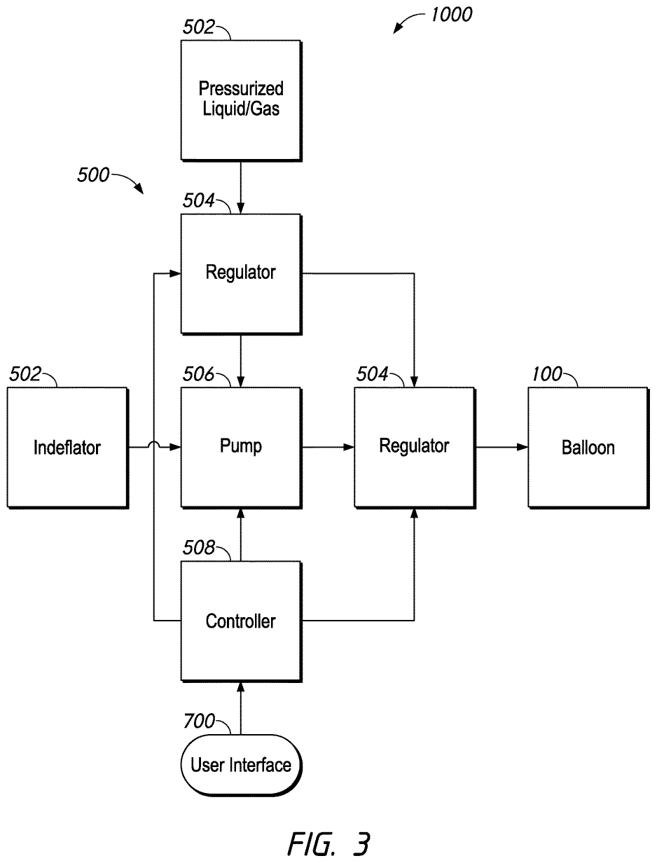

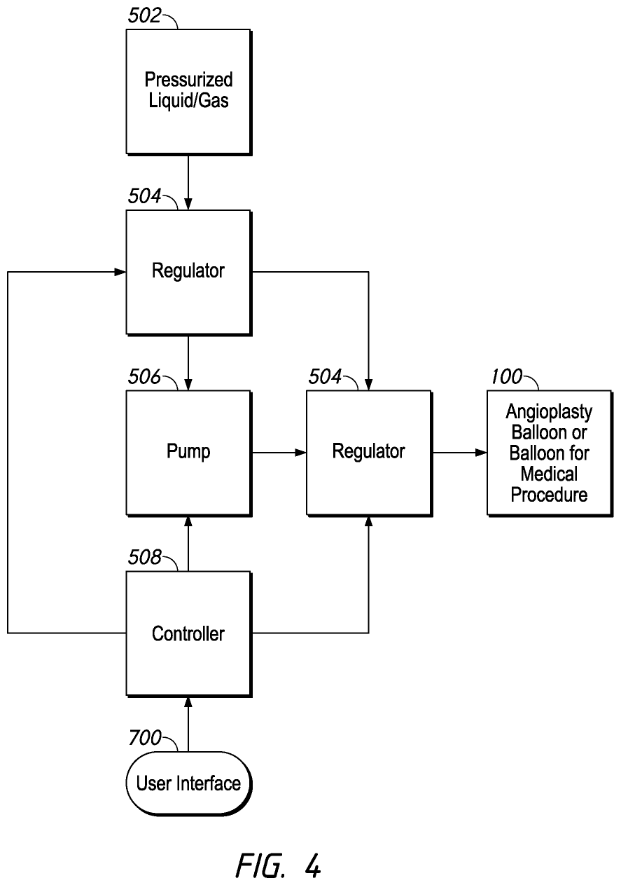

FIGS. 3 and 4 are block diagrams that schematically illustrate the system 1000 according to some embodiments. The system 1000 can include the generator 500 adapted to generate an increase in pressure or a high-velocity pressure wave or burst pressure wave in the fluid for use in pulse wave enhanced balloon angioplasty. The generator 500 can include one or more subcomponents including any subcomponents of the generator 800 described herein. The generator 800 can include one or more subcomponents including any subcomponents of the generator 500 described herein. In some embodiments, the shock pulse of the pressure wave can be very short in duration. The source of fluid 502 may comprise any gas suitable for use in the vascular system, for example, carbon dioxide. The source of fluid 502 can provide pressurized liquid or gas. The source of fluid can be an indeflator. The generator 500 can include a pump 506. The generator 500 can include a compressor 506. The generator 500 can include one or more regulators 504. In some embodiments, the generator 500 comprises a regulator 504 between the source of fluid 502 and the pump 506. In some embodiments, the generator 500 comprises a regulator 504 between the pump 506 and the balloon 100.

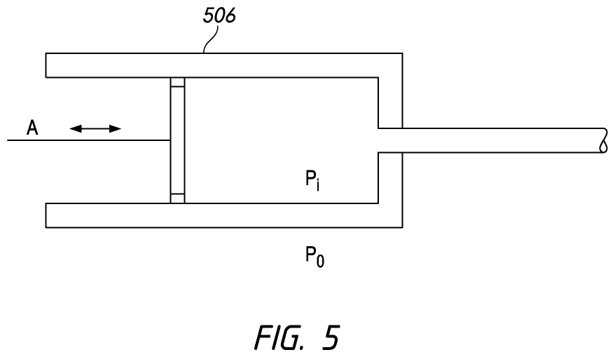

FIG. 5 is an example of the pump 506 of the generator 500. The generator 500 can displace incompressible fluid with a piston. The piston can be movable relative to a cylinder. The pressure pattern can be actuated by movement of the piston as shown by arrow A. The piston can be manipulated to achieve the desired displacement using any pressure pattern described herein.

The source of fluid 502 can pneumatically connect to the pump 506 via the regulator 504. The regulator 504 can be used to regulate the pressure of the input fluid to a desired pressure such as, for example, 100 psi. In some embodiments, the pump 506 can include a hydraulic pressure intensifier that uses the compressed fluid to increase the pressure of fluid received from the source of the compressed fluid. For example, a pressure wave regulator having a 2:1 pressure range ratio can increase and decrease the pressure of the fluid to about 12 ATM using pressurized gas or liquid at 6 ATM psi from the regulator 504. The pressure wave regulator can have pressure range of 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1, or any range of two of the foregoing values. Different pressure intensification ratios may be used in different embodiments, for instance, depending on the pressure of the source and the pressure desired within the balloon 100. By adjusting the fluid pressure with the regulator 504, the pressure of the fluid output from the pump 506 can be selectively adjusted to a desired value or range of values. In some embodiments, the pressure of the fluid can be adjusted within a range from about 2 ATM to about 20 ATM. In some embodiments, a pressure range from about 2 ATM to about 10 ATM can produce effective surface motion, contraction and expansion, at the angioplasty balloon surface that enables particularly effective movement of the raised features 300 of the strip 200 to effectively treat diseased vessel lesions. The pressure within the balloon can spike with the pressure wave to be 4 ATM, 5 ATM, 6 ATM, 7 ATM, 8 ATM, 9 ATM, 10 ATM, or any range of two of the foregoing values.

The source of fluid 502 can include a fluid container such as an indeflator commonly used in angioplasty surgeries holding a medical-grade saline solution, a contrast agent, a solution with chemicals or medications, or any combination thereof. More than one fluid source may be used. In some embodiments, it can be advantageous for pressure wave translation or formation if the liquid provided by the source of fluid 502 is substantially free of dissolved gases (e.g., less than 0.1% by Volume) and particulates, which can act as nucleation sites for bubbles, where the bubbles may limit the effectiveness of translating the pressure wave through the liquid medium. In some embodiments, a bubble detector may be disposed between the source of fluid 502 and the pump 506. In some embodiments, the tubing can be configured to limit diameter changes under high-pressure loads.

The system 1000 can include one or more regulators 504. The regulator 504 may be operable with compressed liquid or compressed gas from the source of fluid 502. The regulator 504 may be operable with the pump 506. The regulator 504 can be used to regulate the pressure of the liquid or gas to a desired value or range of values, like that of a wave pattern. For example, in some embodiments, the regulator 504 reduces the 6 ATM pressure from the pump 506 to about 2 ATM and then raises the pressure to 10 ATM. The regulator 504 may include one or more pressure sensors and/or one or more bleed-off valves (e.g., an air-driven needle valve) to provide the desired pressure and to permit an operator to actuate/de-actuate the pressure wave output.

The system 1000 may include a controller 508 that controls the operation of the components of the system 1000. The controller 508 can control the generator 500. The controller 508 can comprise a microprocessor, a special or general purpose computer, a floating point gate array, and/or a programmable logic device, any software or hardware described herein. In some embodiments, the controller 508 can be used to operate the one or more regulators 504 and the pump 506 so that the range-of-pressure of the fluid delivered is at a suitable working pressure. The controller 508 may also be used to control the system 1000, for example, by limiting system pressures to be below thresholds and/or by limiting the time that the pressure remains in the high pressure state. The controller 508 can be used to control the system 1000 by ensuring a base pressure within the fluid of the system. The controller 508 can control motor speed. The controller 508 can control rotation. The controller 508 can control how frequently the pressure wave is sent. The controller 508 can control parameters of the pressure wave. The controller 508 can increase the intensity of the pressure wave. The controller 508 can control the amplitude of the pressure wave. The controller 508 can control frequency separately from intensity. The controller 508 can regulate the impact energy of each pulse to the balloon. The controller 508 can include a feedback loop. The controller 508 can determine if there is a frequency change. The controller 508 can determine if there is an intensity change. The controller 508 can regulate the range of pressure change. The controller 508 can determine if there is a pressure change. The controller 508 can turn off the motor if there is an intensity change. The controller 508 can turn off the motor if there is a pressure change. In some embodiments, the controller 508 may be used to vary or cycle the pressure of the fluid delivered to the balloon 100, for example, by cycling pressures provided by one or more of the regulators 504. In some embodiments, sinusoidal or sawtooth pressure wave variability, as described herein, can be used to provide corresponding variability in the pressure delivered to the balloon 100. In certain embodiments, cycle time for the pressure variability may be in a range from about 1 Hz to about 1 KHz. In some embodiments, the controller 508 may regulate a pulse intensifier device. A pulse intensifier such as a piezoelectric transducer, can cause pulsations such as a percussion pulse or percussion wave. For example, in some embodiments, the pulse wave comprises a percussive wave, which may include a series of discrete changes in the pressure pulses, like that of a shockwave. The controller 508 can include a communication interface, such as a Bluetooth interface. The controller 508 can include a Bluetooth interface to communicate data from the controller 508 to an external device, such as an app on a phone. There can be Bluetooth data transmission either into and out of the device, both into and out of the device. The data transmission can be for collecting data, such as the number of pulses, the pressure of the system, and at which pulses did the pressure change up or down, or any other data generated by the generator 500.

The controller 508 advantageously can control the amplitude and frequency of the pulsations sent to the balloon 100 and to the serrated strips 200 with the raised features 300. In some embodiments, the amplitude of the pressure variation may be in a range from single digits to triple digits of psi, e.g., 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 200 psi, 300 psi, 400 psi, 500 psi, 600 psi, 700 psi, 800 psi, 900 psi, or any range of the foregoing values. The pulse frequency may be in a range from about 0.1 Hz to about 10 MHz. However, the higher frequencies can be dampened by the length of the catheter. Therefore, some embodiments can be limited to 60 KHz or less, due to the length of the catheter body from the proximal hub 602 and the generator 500 providing the pressure source to the ballon 100.

The system 1000 may also include a user interface 700 that outputs relevant system data and accepts user input. In some embodiments, the user interface 700 comprises a touch screen graphics display. In some embodiments, the user interface 700 may display information including the working fluid pressure and instructions and/or procedures for operating on different disease types (e.g., calcified, fibrin, or a combination). The user interface 700 may accept user input such as a time setting that sets a maximum time during which the system 1000 can deliver pressure wave. In some embodiments, an operator can select a ramp up and/or ramp down option in which the working fluid pressure can be gradually increased or decreased, respectively. The user interface 700 can allow the user to adjust parameters of the system 1000. The user interface 700 can allow the user to adjust the frequency of the pressure waves. The user interface 7000 can allow the user to adjust the amplitude of the pressure waves. The user interface 700 can allow the user to start generating pressure waves. The user interface 700 can allow the user to stop generating pressure waves. The user interface 700 can allow the user to alter one or more parameters as described herein. The user interface 700 can allow the user to visualize a parameter of the system 1000. The user interface 700 can allow the user to visualize what is happening within the balloon 100. The user interface 700 can allow the user to visualize what is happening within the vessel.

The purpose of the inflation device 400 can be to deflate the balloon. In some methods of use, the system 1000 has been purged of air or other gas fluids and can be entirely comprised of incompressible fluid, such as water, saline, or a combination of saline with any commonly used contrast material. In some methods of use, the system 1000 contains the incompressible fluid in semi-rigid components (e.g. rigid plastic inflation device, semi-rigid tubing connections, semi-rigid catheter shaft) except for the therapeutic balloon region 100, which can be intentionally comprised of less rigid and more compliant material. The connector 450 can be configured to enable connectivity of fluid among the three components, including the inflation device 400, the generator 500, and the angioplasty balloon 100 when the inflation device 400 and the generator 500 are separate components. In some embodiments, the system 1000 does not include the connector 450 when the pulsatile pump function is incorporated in the inflation device 400. The inflation device 400 and the generator 500 can be connected to the same port of the catheter in some embodiments. The inflation device 400 and the generator 500 can be connected to the different ports of the catheter in some embodiments.

In some embodiments, the purpose of the generator 500 can be to produce pressure waves. The pressure wave can function as a fluid wave. The pressure wave can compress fluid particles. The pressure wave can travel through fluid at the speed of sound. Unlike many intravascular lithotripsy devices that do not displace a volume, the generator 500 can displace the fluid volume. As the generator 500 increases pressure in the reservoir, a change in the pressure is translated through the catheter body to the balloon 100. As the pressure wave enters the balloon body 100, the regional volume within the balloon increases enabling the balloon membrane to expand, allowing the internal volume of the balloon to increase temporarily during each pressure wave. The volume change continues until the pressure-induced stretching of the balloon wall balances the energy input from the pressure wave. The design of the balloon 100 membranes flexibility can be modified to match the desired balloon expansion during each pulsatile wave. The design of the generator 500 can include a reservoir with enough volume to adequality deliver the necessary volume to match the balloon expansion during each pump event. The design of the generator 500 can include a piston that displaces the system membrane with enough volume to adequality deliver the necessary volume to match the balloon expansion during each pump event. In some embodiments, the pressure waves can change the fluid volume locally within the balloon 100. The pressure wave can locally increase or decrease the fluid within the balloon as the pressure wave propagates. The pressure wave can create slight volume changes within the fluid of the balloon 100. In some embodiments, the purpose of the generator 500 can be to cyclically displace the incompressible fluid within the system 1000 resulting in cyclical displacement of the balloon 100. The cyclical displacement can include a change in diameter and/or length of the balloon 100. The cyclical displacement of the balloon 100 in turn generates a cyclical relative displacement of the serrated strips 200 and the raised features 300. This can be accomplished by the following principles.

The generator 500 can constrain incompressible fluid within a reservoir. The reservoir can be outside of the body of the balloon 100. The reservoir can be within the generator 500 which can be proximal to the balloon 100. The generator 500 can constrain incompressible fluid in a semi-rigid container walls of the reservoir. The semi-rigid container walls can be transparent. The semi-rigid container walls can comprise hard plastic, such as Lexan®. The reservoir of the generator 500 can include one wall or membrane which can be moveable. The movable wall or membrane can be configured to displace a defined volume of fluid when actuated. The generator 500 can include a piston in a cylinder to displace the membrane. In some embodiments, the piston can have o-rings or piston rings to minimize fluid loss when the system 1000 is pressurized.

The generator 500 can have any feature of the systems described herein. In some embodiments, the generator 500 can move the membrane of the generator 500. The membrane can have any features of the membrane described herein. The membrane can be moved. The membrane can be manipulated to achieve the desired displacement using the following movements, alone or in combination. The membrane can be moved by a manual movement of the operator. The operator can push in and pull out a handle connected to the membrane. The movement can be translated to a rotational or other desirable movement by the operator to facilitate more ergonomic effort. The membrane can be moved by means of stored mechanical energy. The stored mechanical energy system can include a spiral torsion spring to enable the cyclical movement (in and out). The spring can have any features of the spring described herein. The generator 500 could be pre-loaded with energy by an operator for each cycle of usage, in some embodiments. While in other embodiments, the generator 500 can be pre-loaded once by the operator and the system uses electrical power to re-load the system with stored energy for generating subsequent pulses. The generator 500 can be provided in a stored energy condition. The membrane can be moved by movement of a rotating cam drive or by a solenoid, or by a piezoelectric stack. The membrane can be moved by movement from a compressed spring or piezo stack. The membrane can be moved by movement powered by electricity (e.g. battery DC or wall AC). The membrane can be moved by movement from a rotating cam shaft. The membrane can be moved by movement from a rotating cam shaft linked to a pin with a spring.

The membrane movement can be quick, shallow movements when a piezoelectric stack or piezoelectric pump is used. For example, the shallow movements can be between a few microns and 100 microns. For example, the movements can be quick in milliseconds, or even microseconds. These pumps can limit the ability for moving volumes necessary to produce a radial expansion at the balloon. The membrane movement can be quick with movements adequate to produce radial expansion for a variety of balloon diameters and length when DC or stepper motors are used. Stepper motors can offer high torque and precision motion control but typically run slow. Additionally, stepper motors can typically require complicated drivers. The drivers for a stepper motor can provide precise pulses of power to certain poles within the motor at a pre-determined rate to make the motor turn. This type of motor can limit flexibility by forcing more complication in the software or hardware design in the drivers to move the motor. A brushless DC motor with an integrated controller can allow for DC power to be applied to the motor to drive it. This setup can be significantly cheaper and less complicated to design. To address the limitation of torque and precision found in stepper motors but not in brushless DC motors, the use of a high geared 12000 RPM motor with a planetary gear box at a 138:1 ratio can drive up the torque and increases the precision of motion in some embodiments. Although other motor designs including combination of motors such as a DC motor in line with a piezoelectric stack are also an option in the design, these alternative can have their own limitations due to increasing overall system complexity.

The membrane can have any structure described herein. In some designs, the membrane can be made of a highly durable and flexible material, for instance silicone. In some embodiments, the membrane can have a shaped design. The membrane can have a central area configured to be struck. The central area can be flexible, such as a plastic or polymer. The central area can be rigid, such as a metal. The central area can be circular. The membrane can have a peripheral area. The peripheral area can be circular. The peripheral area can be flexible, such as a plastic or polymer. The peripheral area can be flexible due to shape. The peripheral area can include one or more flexible regions. The peripheral area can be configured to allow for deformation. The peripheral area can have increased flexibility. The peripheral area can allow for movement. The peripheral area can allow for increased bending. The peripheral area can include one or more corrugations. The peripheral area can include bellows. The peripheral area can include flexible joints. In some embodiments, the membrane can be made of a more rigid material with relief features along the edge of the central cone or area. When the membrane is made of rigid materials, such as a metal or composite, the membrane can add energy to the pressure wave. The membrane can snap up or down when struck. The membrane can snap into the fluid side of the membrane when struck. The membrane can snap into the dry side of the membrane after being struck. The membrane can be designed to occupy one of two distinct energy states. The membrane exists in one or the other energy state depending on its loading condition. The membrane can be any structure that separate the working fluids from the drive mechanism. The membrane can be a flexible diaphragm. The membrane can comprise flexible material such as rubber, PTFE, polyester, or silicone. The membrane can comprise rigid material such as stainless steel, nodular cast iron, iron, tin, aluminum alloys, silicon alloys, aluminum-silicon alloys, steel alloys, molybdenum alloys. The membrane can include a coating such as a fiber-reinforced coating or Ferrostan coatings. The membrane can minimize friction. The membrane can include a rigid metal membrane configured to withstand high loads. The membrane can be a piston membrane. The membrane can be a metallic diaphragm. When metallic diaphragm are considered, the use of circular regions, typically near the rim of the diaphragm, can offer relief and support for the movement of the diaphragm during the pressure wave production. The membrane can endure the loading of the piston under repeated pulses. The membrane can be rigid. The membrane can be semi-rigid. The membrane can seal against the motion of the piston. The membrane can create a suction and discharge. The membrane can be actuated by a piston. The piston can apply a local force on the membrane. The piston can be drive by eccentric cams. The cam-based system can translate rotation movement to linear movement to act on the membrane. The membrane can seal a fluid chamber of reservoir. The membrane can prevent leakage into the area of the piston. The generator can include additional seals or o-rings.

The membrane can have a neutral configuration from which the membrane is moved. The neutral configuration of the membrane can be concave. The neutral configuration of the membrane can be flat. The neutral configuration of the membrane can be convex. The membrane can be cone or dome shaped. The membrane can be a movable wall. The membrane can be a drum. The membrane can be a plate. The membrane can have a flexible rim. The membrane can be bound to a wall. The membrane can have a majority of the surface that is extendable. The membrane can be a movable wall. The membrane can be a drumhead. The membrane can be thin and flexible. The membrane can be semi-rigid. The membrane can be stretched over the reservoir. The membrane can be a plate with a flexible rim. The membrane can be bound to the wall of the reservoir while a majority of the surface of the membrane can be extendable. The membrane can be a diaphragm. The membrane can be struck creating sound waves that resonate within the fluid medium used to fill the balloon 100. The membrane can vibrate in a frequency pattern.

The generator 500 can deliver pressure waves to the balloon 100. The pressure wave can be a fluid wave within the balloon 100. The fluid molecules within the balloon 100 can vibrate in the same direction as the propagation of the wave, from the proximal end of the balloon to the distal end of the balloon. The balloon 100 can be filled with an incompressible fluid. The balloon 100 can be connected to a catheter 600. The catheter 600 can be filled with an incompressible fluid. The catheter 600 can include a proximal hub 602. The proximal hub 602 can include a fluid port valve 604. Fluid from the generator 500 can be delivered to the fluid port valve 604 of the proximal hub 602. Fluid from the inflation device 400 can be delivered to the fluid port valve 604 of the proximal hub 602. The balloon 100 can be configured to be inflated and deflated with the inflation device 400 through fluid delivered to the fluid port valve 604. The balloon 100 can be configured to receive a pulsatile flow from the generator 500 through fluid delivered to the fluid port valve 604. The catheter 600 can extend from the balloon 100 to the generator 500 and/or the inflation device 400. In some embodiments, the catheter 600 can connect to the connector 450. In some embodiments, the generator 500 can connect to the connector 450. In some embodiments, the inflation device 400 can connect to the connector 450.

In some embodiments, the generator 500 can generate a pressure wave that travels from the generator 500, to the fluid port valve 604 on the hub 602 of the catheter 600, along the length of the catheter 600, and to the balloon 100. In some embodiments, the generator 500 can generate a pressure wave from movement of the membrane within the generator 500. In some embodiments, cyclical acceleration, velocity, and/or deacceleration of the membrane and/or piston can be manipulated to result in the desired pressure waves of the fluid medium used to fill the catheter 600 and the balloon 100. Independent of the design of the generator 500, the pulsatile pressure wave can be of many forms, including but not limited to a fluid wave that travels within the body of the balloon and an acoustic wave that travels from the body of the balloon into the tissue. Independent of the design of the generator 500, a defined frequency can be induced within the pressure wave such that the wave is transmitting a frequency in the audible spectrum, the infrasound spectrum, and/or the ultrasound spectrum. The generator 500 can produce more than a single frequency carried on the pressure wave. In some cases, a batch or set of frequencies are carried by a pressure wave. The pressure wave can have a complex, broadband signal. The center frequency of the pressure wave can be the peak or dominate frequency within the range, which can be used to represent the pressure wave. The pressure wave can have several, individual frequency components. The center frequency can be a calculated average allowing the pressure wave to be characterized. In some embodiments, other wave generation systems are contemplated including systems that create a shock event through any variety of techniques, including, but not limited to, the cavitation of a bubble where the static pressure of the liquid in the system reduces to below the liquid's vapor pressure leading to the formation of small vapor-filled cavities in the liquid producing inertial cavitation. When subjected to higher pressure, these cavities, called bubbles or voids, collapse and can generate shockwaves transmitting ultrasonic frequencies inclusive of frequencies below 1 KHz and frequencies above 100 KHz. These cavitation events can occur within the balloon 100 or outside the balloon 100. Sometimes the cavitation event can be produced at the region of the raised features 300. Sometimes the cavitation bubbles are so abundant and confined into the crack or wedge under the raised features 300 that they compress and expand rapidly as the individual raised feature 300 moves into and out of the wedge formed on in the tissue surface. The compression and rarefaction event cycle can promote bubble implosion. In some embodiments, cavitation bubble implosion can generate destructive shockwaves. In some embodiments, cavitation bubble implosion can facilitate crack propagation. In some embodiments, cavitation bubble implosion can facilitate longitudinally oriented cracks along the serrations. The generator can be a handheld pressure regulator in series with the motorized pressure pump regulator. The generator can be a pulse intensifier device. A pulse intensifier such as a piezoelectric transducer, can cause pulsations such as a percussion pulse or percussion wave. The generator can include a miniature array of emitters. The generator can include lithotripsy emitters. The serrations can reduce the number of emitters. The serrations can reduce the number of transducers. The generator can create a localized effect at the treatment site. The generator can discharge energy. The generator can vaporize the fluid within the balloon 100. The generator can create a bubble that expands and contracts. The generator can emit energy from a transducer within the balloon body. Other wave generator systems are contemplated, alone, or in combination with the systems described herein.

The generator 500 can generate pressure waves that can be high-pressure waves of energy, such as burst pressure waves, or blast waves, or shockwaves. The inclusion of these pressure waves can offer advantages over design features that produce waves that are set at a defined pressure and then rest at the pressure for a given amount of time. The generator 500 can generate pressure waves that produce an extremely rapid and intense release of energy. For example, the pressure wave can propagate at the speed of sound. The liquid in the balloon 100 can be much less compressible than gases, and the pressure wave can travel about 4× faster in liquid than gases. The pressure wave can travel at approximately 1520 to 1540 meters per second in the fluid of the system. The pressure wave can travel at approximately 1540 meters per second through soft tissue. The pressure wave can release mechanical energy along the length of the balloon, and beyond the distal end of the balloon. The pressure wave can release energy through molecular movement, accelerating the breakdown of calcium. The pressure wave can release energy through cavitation. For example the system can use 30 V to generate pressure waves. For example, the system can create about 10 ATM to 14 ATM of pressure within the balloon 100. For example, the system can operate at 1 Hz to 3 Hz. For example, the system can create a low energy density less than 0.1 mJ/mm², for example 0.001 mJ/mm² to 0.01 mJ/mm². For example, the system can specifically target calcified lesions while leaving soft tissue undisturbed. In some embodiments, the system 1000, advantageously has the ability to limit the overall length of time to generate fracturing of the disease state by the inclusion of pressure waves. By transmitting a series of pressure waves or burst pressure waves to the serrated strips 200, the system may advantageously generate a serrated line in the tissue in a shorter period of time than when the series of pressure waves or burst pressure waves is not used. Fracture mechanics teaches that an alternating driving force into a surface can be more likely to yield sooner than a static force. That can be the highest pressure being delivered or the highest portion (the peak) of the waveform remains the same. This effect can be amplified when the initiation sites for the fracture, which occur due to the raised features 300 on the strips 200 contacting the surface, are contacted repeatably. When pulsatile wave pressure can be used in place of constant pressure at a given atmosphere, the cellular matrix of the tissue can be repeatably subjected to a load. The repeated impact load can produce a shock wave through the material matrix that with each consecutive impact, and more cellular bonds can break down enabling the vessel to become more compliant. The combination of pressure waves with serration elements can induce a more effective calcium fracturing along a longitudinally-oriented line of weakness with a significant reduction in time. This crack line can be deep enough such that the raised features 300 of the serrated strips 200 no longer contact the vessel. The crack propagates beyond the raised features to penetrate deeper into the tissue. The initiation sites of the crack can join to create the longitudinally-oriented crack aligned along the longitudinal axis of the vessel. The pressure waves can accelerate crack formation. The balloon 100 contacts the vessel wall during delivery of the pressure waves. The pressure waves can expand and contract the lobes of the balloon 100 against the vessel wall. The balloon 100 can apply a tensile force on the vessel wall. As the pressure wave travels through the balloon 100, the surface of the balloon 100 that is in full contact with the vessel wall can pulsate, in regions where no serrated strips 200 are placed, in a pounding like event further cracking and crushing the diseased state of the vessel, including any calcium mass present along the length or diameter of the balloon 100.

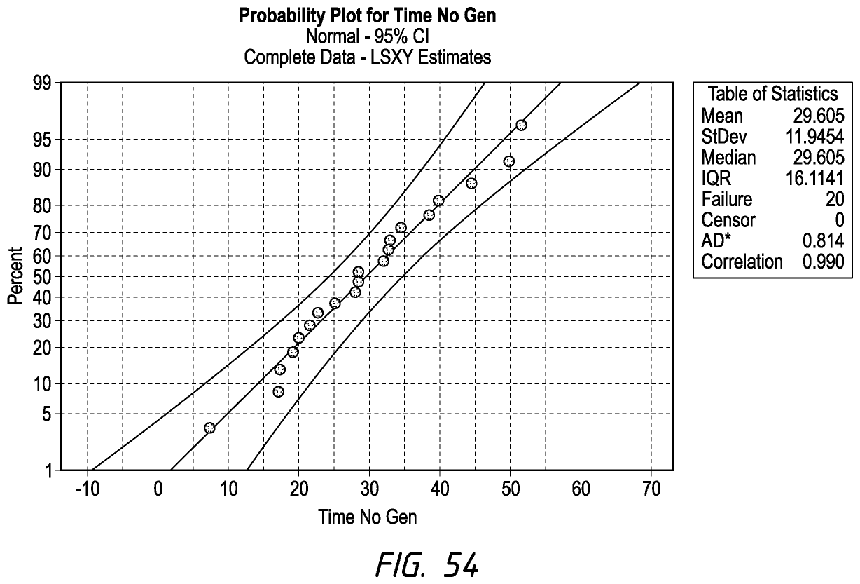

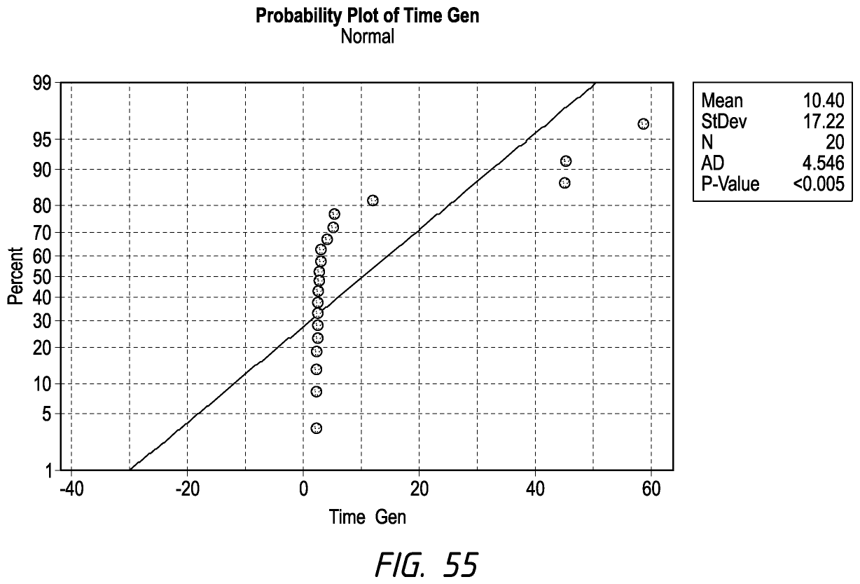

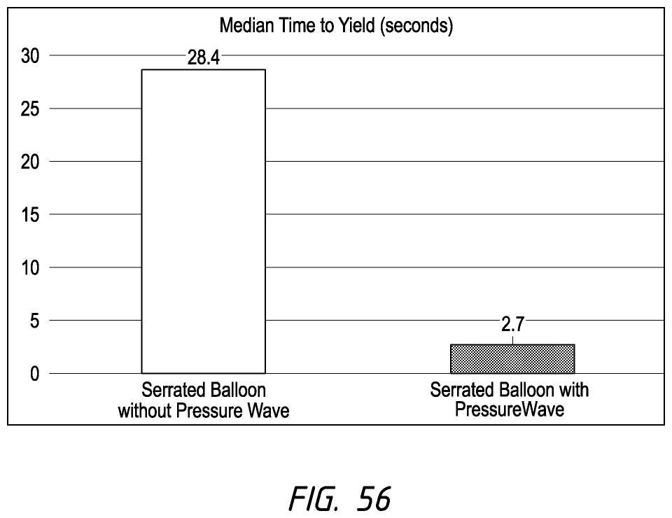

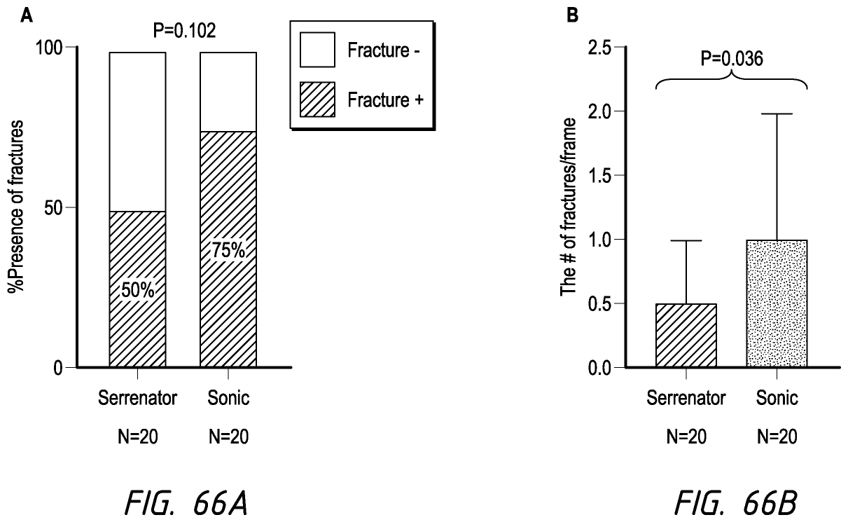

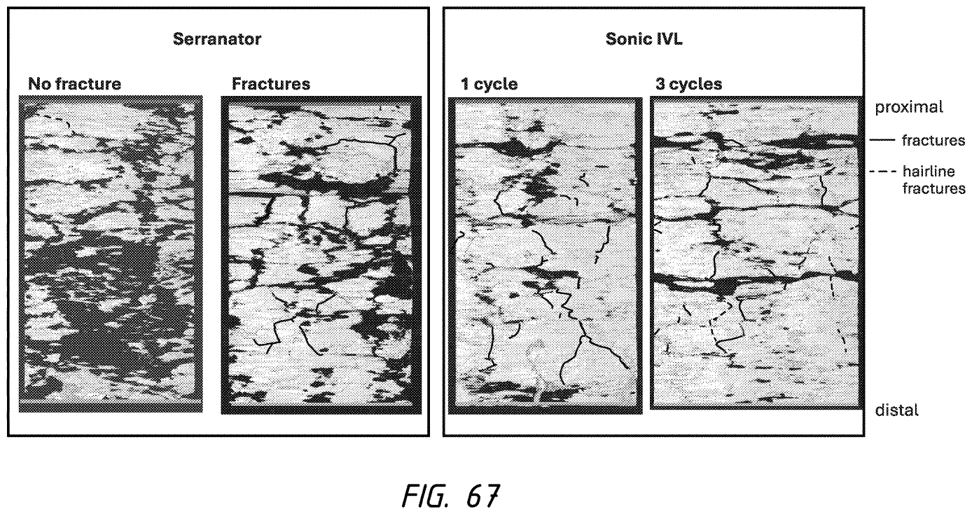

Experiments have been conducted to compare the time needed for effective calcium fracturing by comparing serration angioplasty without pressure wave generation to serration angioplasty with pressure wave generation. Time reduction has been recorded in experiments to drop from over two minutes with serration angioplasty without pressure wave generation to under 2 seconds with serration angioplasty with pressure wave generation to generate effective calcium fracturing. This is a sixty fold decrease in time to generate effective calcium fracturing. FIG. 54 shows additional data with the probability plot with serration angioplasty without pressure wave generation. Less than 5% of samples yielded within 10 seconds with serration angioplasty without pressure wave generation. More than 95% yielded within 55 seconds with serration angioplasty without pressure wave generation. Some samples took even longer to yield with serration angioplasty without pressure wave generation. FIG. 55 shows additional data with the probability plot with serration angioplasty with pressure wave generation. More than 75% yielded within 5 seconds with serration angioplasty with pressure wave generation. FIG. 56 shows additional data, with the median time to yield being 28.4 seconds with serration angioplasty without pressure wave generation and with the median time to yield being 2.7 seconds with serration angioplasty with pressure wave generation. This is a ten fold decrease from the median time to yield. This reduction in time to fracturing can reduce the overall time of the procedure. This reduction in time to fracturing can reduce the time the vessel is occluded by the balloon 100. This reduction in time to fracturing can allow additional vessels to be treated, such heart vessels which cannot be occluded for long periods of time. FIG. 66A shows additional data illustrating the comparison of the percentage of the fracture incidence. The percentage presence of fractures was 50% without pressure wave generation and 75% with pressure wave generation. The percentage of fracture incidence significantly increases with pressure wave generation using the systems and methods described herein. FIG. 66B illustrates the number of fractures in the frame. The number of fractures in the frame was about 0.5 without pressure wave generation and 1.0 with pressure wave generation. The increase in fractures in the frame dramatically increased with pressure wave generation. FIG. 67 are summary images of the fracture on the flattened outside view. The views on the left are without pressure generation and the views on the right are with pressure wave generation. There is an increase in the number of fractures with pressure wave generation. There is also an increase in linearity with the fractures with pressure wave generation. These and other data are described herein.

The system 1000 can deliver pressure waves to achieve specific results. In some embodiments, the system 1000 can deliver shockwaves to the surrounding tissue. The system 1000 can perform intravascular lithotripsy to fracture calcium in arteries and veins. The system 1000 can use pressure waves to break down calcified plaque in blood vessels. The system 1000 can use pressure waves to break plaque. The system 1000 can use pressure waves to make it easier to dilate the vessel. The pressure waves can be focused on the plaque. The system 1000 can use pressure waves to create microfissures within the plaque.

In some embodiments, designs for delivering pressure wave or burst pressure waves outlined herein include periods where the serrated structure, such as the plurality of strips 200 with raised features 300, can be moved due to the change in pressure of the balloon 100. The periods of time can be sequential. The periods of time can overlap in whole or in part. The plurality of strips 200 with raised features 300 can be moved via the pressure wave that moves longitudinally along the length of the balloon. These periods with movement of the raised features 300 include the growth or balloon expansion period, the crushing or cracking period, the penetration or serration period, and the recovery or retraction period, which are described herein.

In some embodiments, the pressure wave can be delivered during the growth or balloon expansion period. In some embodiments, the pressure wave can be delivered after the growth or balloon expansion period. The growth or balloon expansion period can correlate with the unfolding and initial penetration of the raised features 300, in some embodiments. The growth or balloon expansion period can correlate with the crack propagation of the microperforations caused by the raised features 300, in some embodiments.

In some embodiments, in both the cracking period and the serration period, the strips 200 with raised features 300 have the propensity to generate a crack with a depth such that the raised features 300 no longer contact the vessel wall. When a crack is generated, the energy delivered within the cracking and serration period can be delivered to the vessel wall. The energy can travel through the balloon 100 that sits between the strips 200. In some embodiments, two distinct and separate forms of energy can be delivered in these periods. The first form of energy can be the mechanical energy of movement produced by the volumetric change of liquid filling the balloon 100. This fluid wave can be generated by the pressure wave. The second form of energy can be the acoustic wave entering the vessel wall through the surface of the balloon 100 and through the strips 200 with raised features 300 coupled to the surface of the balloon 100. This acoustic wave can be generated by the pressure wave. The surface of the balloon 100 oscillates larger and smaller in a pounding or vibrational behavior. This pounding or vibrational effect from the motion of the balloon 100 can be more effective at vessel remodeling than designs without the generation of pressure waves. This pounding or vibrational effect from the motion of the balloon can be more effective in designs with raised features 300 than in designs without raised features 300 due to the nucleation points of a set of serrated lines created by the raised features 300 that initially produce a set of cracked or crushing planes. The pounding or vibrational effect from the motion of the balloon 100 can accelerate crack formation. The pounding or vibrational effect from the motion of the balloon 100 occurs after the microperforations are created by the raised features 300 in some embodiments. With the plaque circumferentially serrated or broken with crack propagation, the arcs of calcium or calcified plaque between the serrated lines can more easily be crushed or pounded into smaller pieces by the pounding of the surface of the balloon 100.

In some embodiments, the balloon 100 can be slowly expanded to contact the vessel wall with the strips 200 and raised features 300 seated into the vessel wall prior to the pressure wave initiating. This can be the growth or balloon expansion period in which the raised features 300 connected to the strips 200 can be pressed perpendicularly against the vessel wall. The raised features 300 of the strips 200 can be positioned in linear rows aligned longitudinally with the vessel to enable effective crack propagation in some embodiments. The raised features 300 of the strips 200 can be positioned in linear rows aligned longitudinally with the vessel enabling vessel compliance with minimal cellular injury in some embodiments. The spacing and linear nature of the strips 200 provides for effective orientational delivery of the pressure wave to the vessel. The spacing of raised features 300 that have a length longer than their width where the length can be oriented in a similar plane as the growth plan for the intimal cells offers advantage to minimizing damage to the vessel wall. When these oriented, spaced apart raised features 300 are combined with the pressure wave, the effectiveness of the combined pulsed enhanced and serration system can be magnified. Thereby the system of the balloon 100, the strips 200, and the raised features 300, combined with the generator 500, allows each of the balloon 100, the strips 200, and the raised features 300 to contribute to the overall lithotripsy outcome. The generator 500 can generate the pressure wave providing all the necessary energy to generate an effective lithotripsy event.

In some embodiments, the generator 500 can include the reservoir, wherein fluid contained within the reservoir is disturbed by the pressure wave. The reservoir can be a chamber, a funnel, or trumpet like shape that directs the pressure wave toward the catheter 600 and the balloon 100. The shape of the reservoir of the generator 500, where the wave is generated, can act like a conversion point and the catheter 600 acts like the throat of a nozzle. The catheter 600 can be the narrowest cross-sectional area of the system 1000 for the flow of fluid. When applying a pressure wave through a funnel to a nozzle, the speed of the pressure wave increases. Due to the contraction of the incompressible fluid through the nozzle and in the system 1000, wherein the reservoir can be the funnel and the throat of the nozzle can be the catheter 600, therefore the speed of the pressure wave increases as seen at the balloon 100. Therefore, the flow can accelerate and can increase in relative speed as the flow exits the catheter 600 and fills the balloon 100. In some embodiments, the pressure wave accelerates as the pressure wave exits the catheter 600. In some embodiments, the pressure wave accelerates as the pressure wave travels from the reservoir of the generator 500 into the catheter 600. In some embodiments, the pressure wave decelerates as the pressure wave travels along a proximal cone of the balloon 100 due to the larger area of the balloon 100 compared to the catheter 600. In some embodiments, the pressure wave maintains a speed across the body of the balloon 100. In some embodiments, the pressure wave accelerates as the pressure wave travels through the distal cone of the balloon 100 due to the smaller area of the cone compared with the body of the balloon 100.

In some embodiments, pressure wave can be delivered during any period of the procedure to facilitate crushing or cracking of the calcium. The crushing or cracking can correspond with the delivery of a single pressure wave, or the repeated delivery of pressure waves by both the balloon 100 and the strips 200 with raised features 300, in some embodiments. The crushing or cracking can correspond with the delivery of one or more pressure waves to crush or crack the calcium or calcified plaque in addition to serration. The crushing and cracking can be circumferential with both the balloon 100 and the strips 200 with raised features 300 delivering pressure waves. The crushing and cracking can be along the entire length of the balloon 100 with both the balloon 100 and the strips 200 with raised features 300 delivering pressure waves. The crushing and cracking can be beyond the length of the balloon 100 as the pressure waves travel distally from the distal tip of the balloon 100. In some embodiments, the use of different frequencies and different wave intensities can produce different effects on the calcium including but not limited to cracking and crushing. In some embodiments, the pressure wave can include ultrasonic frequencies that break the bonds that hold the calcium together. In some embodiments, the pressure wave can include lower frequency waves, such as acoustic waves. These lower frequency waves can contribute to crushing or cracking by pulsating the balloon 100 and strips 200 with raised features 300 against the vessel wall.

In some embodiments, sufficiently increasing balloon pressure through the pressure wave can initiate contact between the raised features 300 of the strips 200 and the wall of the vessel. This initial contact can be distributed across a very small area, such as the leading surfaces of the raised features 300. Thus, the corresponding pressure applied by the raised feature 300 can be a multiple of the initial pressure such as 50×, 100×, 1000×, 1500×, wherein the multiplication is related to the inverse of the surface area difference between the balloon body and the strip tip. As the pressure waves continue, the raised features 300 of the strip 200 may initiate a crack in the plaque with a depth such that the raised features 300 no longer contact the vessel wall. This crack depth corresponds to a crack width, which can be simultaneously driven to open further radially due to the expanding balloon 100 on the entire vessel diameter. When such a crack is initiated, the pressure wave can be distributed to the vessel wall through the body of the balloon 100 that sits between the serrated strips 200. The surface of the balloon 100 can oscillate in pressure and diameter in a vibrational behavior until sufficient cracks under load are initiated.

In some embodiments, the pressure wave can be delivered during any period of the procedure to facilitate penetration or serration. The penetration or serration can correspond with the delivery of a single pressure wave, or the repeated delivery of pressure waves by both the balloon 100 and the strips 200 with raised features 300, in some embodiments. The pressure wave can facilitate penetration of the raised features 300 at low pressure. The pressure wave can facilitate the formation of nucleation sites. The nucleation sites can be formed quicker with pressure wave generation than without pressure wave generation. The pressure wave can facilitate the crack propagation. The cracks can propagate quicker with pressure wave generation than without pressure wave generation. The pressure wave can facilitate longitudinally oriented cracks. The pressure wave can concentrate energy along the strip 200. The pressure wave can concentrate energy to the raised feature 300. The concentrated energy can facilitate the longitudinal nature of the crack propagation. The pressure wave can concentrate energy to prevent or limit random crack formation. The vibrational effect from the motion of the balloon 100, due to the balloon's pressure changes, can be more effective with raised features 300 than in designs without raised features 300 due to the effect of a set of serrated lines initially producing a set of cleavage planes. The lines of serration create cleavage planes that facilitate the crushing of plaque with the pressure wave along the cleavage planes. The cleavage planes can direct the energy from the pressure waves to crack or crush calcium along longitudinally oriented lines. With the plaque circumferential broken by the serrated lines, the arcs of calcium between the serrated lines can more easily be crushed or pounded into smaller pieces by the pressure waves.

The raised features 300 can be a mechanical interface for pressure wave to be translated through to the plaque. The raised features 300 can be serrated. The raised features 300 can rise above the surface of the balloon 100. The raised features 300 can focus the pressure wave. The raised features 300 can focus and multiply the effect of the pressure wave. The raised features 300 can focus and multiply the effect of the acoustic wave that travels through the raised features 300 and into the tissue. The raised features 300 can change surface area that the acoustic wave is applied from the whole balloon 100 with the strips 200 and the raised features 300 to only the outward facing surface of the raised features 300. The raised features 300 can have a small surface area compared with the surface area of the balloon 100. The raised features 300 can concentrate the acoustic wave through the raised features 300 to the calcified plaque. The raised features 300 can be impaction surface. The raised features 300 can have a harder, less compliant material than the balloon 100. The raised features 300 can effectively transmit an impaction force from the balloon 100 to the tissue. The raised features 300 can focus the fluid wave that travels along the body of the balloon 100. The raised features 300 can create an amplitude effect of the fluid wave. The raised features 300 can create a greater propensity for fracturing of the calcium or calcified plaque with the pressure wave.

The system 1000 can provide a relatively large axial coverage of lesions. The system 1000 can provide lithotripsy along the entire length of the balloon 100. The system 1000 can provide lithotripsy along the entire circumference of the ballon 100. The system 1000 can provide lithotripsy through the lobes 120 of the balloon 100. The system 1000 can provide lithotripsy through the strips 200 with the raised features 300. The system 1000 can be effect for 360 degree treatment. The system 1000 can provide treatment for full-circle calcium. In comparison, devices which provide a shockwave may be severely limited in the area of coverage. For instance, these systems provide a relatively small coverage of lesions. In comparison, devices which provide a shockwave may be severely limited in the effective translation of energy due to the amorphous structure of the disease state. For instance, these systems provide a relatively poor translation of energy to tissue beds with a wide range of impedances. These systems rely on the positioning of electrode pairs, and may require several additional electrode pairs to provide axial coverage, but may not cover the entire length of the balloon and may not cover the entire circumference of the balloon. These systems may require multiple high intensity pulses to deliver an adequate amount of energy to effect the diseased tissue, and the energy may be damaging to healthy tissue while the energy may be reflected off highly diseased dense bone like hardened calcified plaque. These disadvantages can be resolved with systems and methods described herein. The system 1000 can provide pressure waves that travel the entire length of the balloon 100. The pressure waves can create a fluid wave that travels distally within the fluid that fills the balloon 100. The pressure waves can create an acoustic wave that passes through the body of the balloon 100. The acoustic wave can deliver ultrasonic frequency along the entire length of the balloon 100. The system 1000 can provide pressure waves that travel beyond the entire length of the balloon 100. The pressure wave can travel distally beyond the distal end of the balloon 100. The pressure wave can travel through fluids, both inside and outside of the balloon. The pressure wave can propagate through the fluid as an acoustic wave. The acoustic wave can travel through the wall of the balloon 100 and through the strips 200 with the raised features 300. The acoustic wave can travel through the mediums. The acoustic wave can travel from the fluid of the system, through the balloon 100 with the strips 200 and the raised features, and into the tissue. The acoustic wave can travel outward into tissue. The acoustic wave can propagate by causing particle-to-particle interactions. The acoustic wave can travel through interfaces of different materials. In some embodiments, the acoustic wave may not be limited to the bounds of the system such as the bounds of the balloon 100. The acoustic wave can carry energy from one point to another. The pressure wave can propagate through the fluid as a fluid wave. The fluid wave can move within the fluid of the system. There can be fluid motion because the pressure change can create fluctuations in density and velocity within the fluid of the system. The pressure wave can induce fluid movement. In some embodiments, the fluid wave can be limited to the bounds of the system such as the bounds of the balloon 100. The fluid wave can be stopped by a solid surface of the balloon 100. The balloon 100 can act as a boundary that the fluid cannot pass though. The fluid wave can carry energy from one point to another. The fluid wave can cause the balloon 100 to vibrate. The fluid wave can cause the balloon 100 to expand. The fluid wave can cause the strip 200 to move in a wave. These and other properties of the pressure waves are described herein.

The system 1000 can provide pressure waves. The pressure waves can travel through a medium including the fluid used to fill the balloon 100 and the tissue disposed near the balloon 100. The pressure wave can include ultrasonic frequency. This ultrasonic frequency can be used to break the bonds of calcium. In some embodiments, ultrasonic frequency can be used instead of subsonic or acoustic frequencies, in some embodiments. The pressure waves can have a wide spectrum of frequencies, with each frequency impacting the vessel in a specific manner. The pressure waves can be equally distributed along the length of the balloon and along the circumference of the balloon. Unlike other systems, the pressure waves do not create localized hotspots of energy. Instead, the energy can be evenly and equally distributed.

The system 1000 can provide pressure waves for disrupting or cracking calcified regions within a vessel, alone or in combination with serration. The pressure waves create disruptive effects which may be synergistic with serration. The pressure waves can contact both non-calcified regions of the vessel and calcified regions of the vessel. The pressure waves created by the balloon 100 can pulse against the non-calcified regions causing no harm to the vessel wall. The pressure waves can pass through healthy tissue. The pressure waves from the system 1000 can extend partially into the calcified regions. The pressure waves from the system 1000 can extend entirely through the calcified regions. The pressure waves can break up hard calcified material.

The system 1000 can generate a pressure wave. The system 1000 can generate a traveling pulse of pressure that moves through the fluid within the balloon 100. The pressure wave can move generally away from the generator 500 from which the pressure wave originates. This pressure wave may comprise a symmetrical expansion shape around the balloon 100, in some embodiments. This pressure wave may comprise an asymmetrical expansion shape around the balloon 100, in some embodiments. The pressure wave travels through the material of the balloon 100 and the material of the strip 200 to impact the tissue and calcified regions outside of the balloon 100.

In some embodiments, the pressure wave carries a single frequency. In some embodiments, the pressure wave carries more than one frequency. In some embodiments, the pressure wave can carry a combination of different frequencies. In some embodiments, the pressure wave carries an infrasonic frequency below 20 Hz. In some embodiments, the pressure wave carries an acoustic frequency between 20 Hz and 20 kHz. In some embodiments, the pressure wave carries an ultrasonic frequency between 20 kHz and 200 kHz. In some embodiments the pressure wave frequencies can include acoustic and ultrasonic frequencies. In some embodiments, the pressure wave carries an acoustic wave that can deliver energy separately from other components of the pressure wave, such as a fluid wave. The acoustic wave can carry one or more infrasonic frequency, one or more acoustic frequency and/or one or more ultrasonic frequency. The inclusion of an acoustic wave with a combination of frequencies can offer advantages. For instance, high frequency acoustic waves, such as ultrasonic waves, can transmit a portion of the pressure wave's energy beyond a transition of materials. The acoustic wave can travel through the balloon 100, the strips 200, and the raised features 300. Translation of frequencies through mediums can be calculated using the reflection coefficient at each interface that the acoustic wave encounters.

$${( \frac{{Z}_{medium 1} - {Z}_{medium 2}}{{Z}_{medium 1} + {Z}_{medium 2}} )}^{2}$$

There can be a transmission effect of the acoustic wave as the wave travels through the system and into the surrounding body.

In some embodiments the use of higher frequency, low amplitude ultrasonic waves, such as those above 1 MHz, can produce a higher friction like motion at the interface between the raised features 300 and the tissue. This high frequency motion can generate high contact friction which can be used to atherectomies the tissue it is in contact with.

In some embodiments, pressure wave can be delivered during any period of the procedure including when the raised features 300 are retracted or in recovery. The pressure wave can be delivered when the serrated strip 200 with raised features 300 is at least partially retracted, in some embodiments. The pressure wave can be delivered before penetration of the raised features 300. The pressure wave can be delivered during penetration of the raised features 300. The pressure wave can be delivered after penetration of the raised features 300 when the raised features fall away from the penetration. The void created by the penetration can be the site of cavitation. The pressure wave can create and implode bubbles within the nucleation site. The pressure wave can create cavitation when the raised features 300 are retracted. The raised features 300 can be retracted under the motion of the fluid wave. The raised features 300 can be retracted with the fall of pressure back to base pressure. The energy delivered can include vibrations from the pressure waves which can travel through the body of the balloon 100. The balloon 100 can vibrate when the raised features 300 are retracted. The vibrational effect from the motion of the balloon 100 can be delivered when the serrated strip 200 is at least partially retracted, in some embodiments. The vibrational effect from the motion of the balloon 100 can be delivered independently of serration. The vibrational effect from the motion of the balloon 100 can be delivered before serration. The vibrational effect from the motion of the balloon 100 can be delivered after serration. The vibrational effect from the motion of the balloon 100 and the penetration of the raised features 300 can be performed in an alternating manner. The vibrational effect from the motion of the balloon 100 and the penetration of the raised features 300 can be performed at the same time. The vibrational effect from the motion of the balloon 100 and the penetration of the raised features 300 can produce a synergistic result. The balloon 100 can deliver acoustic waves when the when the raised features 300 are retracted. The acoustic waves can radiate from the balloon 100 along the circumference of the vessel. The acoustic waves can radiate along the length of the balloon 100, and beyond the distal end of the balloon 100. The acoustic waves can be directed from the balloon 100 to the retracted raised features. The acoustic waves can be directed from the base of the strip 200 to the retracted raised features 300. The energy can be concentrated to the raised features 300. The raised features 300 can deliver amplified energy to the void when advanced into tissue. The raised features can deliver energy from both the strip and the adjacent lobes of the balloon when advanced.

In some embodiments, the inclusion of pulse wave technologies applied to coronary vessels can be configured to reduce the amount of time required to produce adequate lumen gain and effective vessel compliance. The embodiments can be configured to accelerate the effectiveness of serrations, which would be of benefit since coronary vessels have a biologic limitation to the inflation time of angioplasty balloons that constrict blood flow. Pulse wave enhanced angioplasty can be advantageous to reducing the time needed to generate effective lumen gain. During the cycling event, the serration intravascular lithotripsy system 1000 can produce three modes of energy delivery. The first mode of energy delivery can be the pressure wave acting as an acoustic wave which moves through each progressive medium delivering a proportional component of the wave energy relative to the reflection coefficient of each medium it passes through. The second mode of energy delivery can be the pressure wave acting as a fluid wave that produces a volumetric change in the balloon that expands the balloon nearly matching the growth of the balloon at both the upper and lower boundaries of pressure corresponding to the balloon's compliance curves. The second mode of energy delivery can function as an alternating pressure gradient. The second mode of energy delivery can move as a wave along the length of the balloon. The third mode of energy can be the impact of the raised features 300 that sit proud of the balloon surface which individually and collectively act like magnifying points for both the first mode of energy delivery and the second mode of energy delivery. For instance, the second mode of energy and third mode of energy in combination produce a magnified pressure between 1,000 to 1,500 times the energy of the fluid wave. The magnification amount can depend on the number and periodicity of the raised features 300 on each strip 200. This significant magnification can be useful for accelerating the speed at which fracturing and vessel compliance occurs while still maintaining a propensity for the fracture to occur in one or more longitudinally oriented serrated lines.

This alternating pressure gradient of the second mode of energy, in combination with strips 200 and raised features 300 extending from the balloon surface of the third mode of energy, can result in a more rapid and effective initiation, formation and propagation of cracks in the intima, internal elastic lamina, and medial tissues. The crack formation and propagation can follow material fatigue principles. The fatigue failure of materials such as atherosclerosis or plaque can be more vulnerable when subjected to cyclic, non-static loading than when subjected to static loading. The pulse wave enhanced serration angioplasty system 1000 thereby applies this principle of dynamic loading. This cyclic, dynamic loading through the pressure wave generation can effectively immiserates or pulverizes many different materials including all forms of atherosclerosis, where alternating loads or stresses have a much more substantial impact on accelerating fatigue failure than static loads or stresses.

The concept of balloon angioplasty with the inclusion of wave technology can be referred to as intravascular lithotripsy (IVL). In some embodiments, lithotripsy can be described as calcified plaque modification. In some embodiments, lithotripsy can use waves to fracture calcium bonds. In some embodiments, lithotripsy can utilize pressure waves. In some embodiments, lithotripsy can utilize acoustic waves. In some embodiments, lithotripsy can utilize burst pressure waves. In some embodiments, lithotripsy can utilize shockwaves. In some embodiments, lithotripsy can utilize any wave described herein. In some embodiments, lithotripsy can utilize acoustic waves, which interact with the calcium to cause multiplanar fractures. In some embodiments, lithotripsy can utilize emitters to employ an electric spark within the balloon body. The sparks produce expansion and collapse of bubbles within the balloon that results in production of acoustic pressure waves, in some embodiments. The study of the interaction of bubbles with shock waves and ultrasound can be sometimes termed acoustic cavitation. Lithotripsy concepts can include peak-positive and peak-negative pressure, rise time, pulse duration, energy flux density, as well as different definitions of focal zones. These lithotripsy concepts can be applied to pulse wave serration angioplasty systems as described herein.

The system 1000 can produce pressure waves for lithotripsy. These pressure waves can travel through soft tissues with minimal effect due to the nature of soft tissue and due to the reflection coefficient of the tissue. These pressure waves have fracturing effects when these pressure waves encounter tissues with differing impedances or reflection coefficients, such as the transition from soft normal tissue to hard calcified tissue. Lithotripsy technologies that use pressure waves to act upon the calcium can be configured to produce pressure waves that are transmitted both circumferentially and transmurally in an unfocussed manner. Lithotripsy technologies can use pressure waves to pass through thin layers of soft tissue and have an effect on both superficial and deep lying calcium. Lithotripsy technologies for coronary intervention can produce pressure waves with relatively lower positive peak pressures (50 atm versus 1000 atm) with a negligible or absent trailing peak negative pressure (0 atm versus −150 atm). Lithotripsy technologies that use pressure waves for renal intervention can have higher positive peak pressures and/or trailing peak negative pressure. Lithotripsy technologies that use pressure waves for coronary intervention can result in a tensile stress below the threshold associated with tissue damage. Thus, the leading edge of the pressure wave can impart compressive stress to calcium and the compressive stress can be the primary mechanism of calcium fracture. The compressive stress can be amplified when the pressure wave is combined with stress concentrators, such as the raised features 300 on the surface of the balloon 100. Lithotripsy technologies that use pressure waves can limit the potential for soft tissue damage wherein the soft tissue may have similar impedance parameters of the fluid in the balloon 100.

The pressure waves can create a hammering motion with a certain frequency. The hammering motion can convert potential energy to kinetic energy to strike a surface. The potential energy can be from a strike surface being moved into and out of contact with a surface. The potential energy can be from the radially outwardly facing surface 304 of the raised feature 300 being moved into and out of contact with a surface of the vessel. The hammering can include a repetitive back and forth linear action used to deliver an impact force. The hammering motion can be characterized by rhythmic pulses. In some embodiments, the hammering motion can be distinct from continuous vibrations. The hammering motion can include directional force application, such as outward from the balloon 100. The hammering motion can maximize energy transfer through impact. For example, percussive hammering motions can be highly efficient at transferring energy to break or penetrate hardened material such as calcified material. The hammering motion can covert kinetic energy into stress waves. The stress waves can propagate through the raised feature 300 directly to the calcium. The hamming motion can create fractures and fragmentation rather than abrasion. The impact of the hammering motion can propagate through the raised feature 300 to the material directly in contact with the raised features 300, such as the calcified deposits. While axial stress waves are the primary stress waves, the interaction of the raised features 300 with the calcium can also induce vibrations in certain directions. The stress waves can produce crushing and fragmentation of the calcium. In some embodiments, the raised features 300 of the plurality of strips 200 can transfer energy at the speed of sound. In some embodiments, the raised features 300 of the plurality of strips 200 can transfer energy. The pressure waves can cause the balloon 100 which is in contact with the vessel wall to produce a hammering motion. The pressure waves can cause the strips 200 with the raised features 300 to produce a hammering motion. In some embodiments, the frequency of the hammering motion can be independent of the speed of the pressure wave carried during each cycle. In some embodiments, the hammering motion of the balloon 100 with the strips 200 and raised features 300 can be measured in the number of pulses per second. In some embodiments, the pulsations of the balloon 100 can be 1 Hz, 2 Hz, 3 Hz, 4 Hz, 5 Hz, 10 Hz, or any range of two of the foregoing values. In some embodiments, each pulsed wave of the balloon 100 can include a portion of the sonic frequency in the ultrasonic frequency range above 20000 Hz. In some embodiments, the pulsations of the balloon 100 can include frequency bands in the 5 kHz, 10 kHz, 20 kHz, 25 kHz, 30 kHz, 35 kHz, 40 kHz, 45 kHz, 50 kHz, 55 kHz, 60 kHz, 65 kHz, 70 kHz, 75 kHz, or any range of two of the foregoing values. The system 1000 can utilize pulsations after the balloon 100 is inflated. The pulsations can be different than the typical inflation and deflation cycle that occurs during a procedure. Rather, the pulsations can be directed mechanical energy that generates pressure waves while the balloon 100 is inflated. The pulsations of the balloon 100 with the strips 200 and raised features 300 can occur rapidly and repeatedly.

The pressure waves can include a static pressure and a dynamic pressure. According to Bernoulli's law, the total pressure in a flowing fluid includes static (e.g., thermodynamic) pressure plus dynamic pressure (associated with fluid kinetic energy). The ratio of static pressure to dynamic pressure can vary depending on the type of system that generates the pressure waves. Static pressures in motor driven pump systems may be less than static pressures in compressor systems, because the motor-driven pump primarily increases the dynamic pressure (e.g., the fluid velocity) of the liquid. The total pressures, including static pressure plus dynamic pressure, and the pressure waves generated therein can be implemented with variety of embodiment designs, such as those utilizing either compressor systems and/or pump systems. Embodiments of various systems are described herein.

The effectiveness of using pulse wave technology to further enhance serration angioplasty can be compared with percussion-based methods, such as Churn drilling, which can be a percussive drilling method where the drilling tool is repeatedly raised to a certain height and then released to fall freely onto the rock. The hammering motion of the pressure wave can be comparable to this percussion-based method wherein the raised feature 300 is raised and lowered to fracture calcium through impact. Pulse wave serration angioplasty therein creates a series of impacts not by a free-falling drill bit but by induced pressure waves delivered to balloon 100 that break the diseased tissue allowing the linear line of raised features 300 to advance deeper into the diseased tissue with each consecutive wave of pressure. Churn drilling can be characterized by its simplicity and effectiveness in drilling through hard rock formations. Likewise, the application of pulse wave serration angioplasty offers an increased level of efficiency, which can be defined as the fraction of impact energy delivered to the diseased vessel tissue. The pulse wave technology described herein can have high efficiency by transmitting a large fraction of impact energy from the balloon 100 with the strips and raised features 300 to the tissue.

The effectiveness of using pulse wave technology to further enhance serration technology can amplify the impact force of the raised features 300. As the pressure wave translates through the medium used to fill the balloon 100, the pressure wave induces a translation of the strip 200 with the raised features 300. In some embodiments, the raised features 300 can have outward facing surface features such as blunt surfaces. In some embodiments, the raised features 300 can be wedge dissectors. Independent of the shape of the raised features 300, the motion of the strip 200 with the raised features 300, which rise above the balloon surface, can be short discrete events in the range of ultrasound frequency pulses. The movement of the raised features 300, during the pressure wave, can be in the micro or nano scale. Independent of the scale in time or amplitude, the effectiveness of the pressure wave when combined with the raised features 300 can be amplified on the disease morphology more thoroughly. The raised features 300 can move at a high speed in the sonic or ultrasonic frequency. The effect of interface contact between the raised features 300 due to the pressure wave and the static diseased tissue can induce an impact shock force. The effect of interface contact between the raised features 300 due to the pressure wave and the static diseased tissue can induce an enhanced effectiveness in rapidly separating the tissue at the points of impact of the raised features.

The system 1000 can include key advantages related to pulse wave serration angioplasty. The system 1000 can advantageously allow for reduced time scale. The pulse wave serration angioplasty can include several time scales that characterize the dynamics of pulse wave serration angioplasty, such as the travel time of raised features 300, the response time of the plaque interface, and the duration of the pulse generated by the generator 500, among other characteristics.

The system 1000 can advantageously allow for efficiency of energy delivery. The energy transfer efficiency depends on many elements. The elements can include the frequency of the waveform, the periodicity of the wave, the intensity or amplitude of the waveform, the acoustic impedance Z of the disease elements, that can be the disease state including the degree and the location of calcium burden, and the ratios of the wave to the elastic properties of the diseased tissue, the compliance of the balloon, the compliance of the catheter tubing and connections, and the geometry and material of the raised features 300, among other characteristics. In some embodiments, maximum performance can be likely to be achieved with circumferential calcium rings in the lumen or with minimal depth below the adventitia or the medial layers of the vessel wall.

When the pressure wave comprises frequencies in the ultrasound range, the pressure wave can carry energy that can be absorbed by the medium carrying the pressure wave, producing effects that vary with intensity. In some systems, ultrasonic energy focused at intensities of 103 to 105 W/m² can be used to shatter or pulverize tissue in surgical procedures. Intensities this great can damage individual cells, potentially causing their protoplasm to stream inside them, altering their permeability, or rupturing their walls through cavitation. In some systems, cavitation can be the creation of vapor cavities in a fluid—the longitudinal vibrations in ultrasound alternatively compress and expand the medium, and at sufficient amplitudes the expansion separates molecules. Most cavitation damage can be done when the vapor cavities collapse or implode, producing even greater shock pressures. In some embodiments described herein, the system 1000 can advantageously avoid the drawbacks of other systems. Alternative approaches to generation of cavitation are described herein

When an object moves at speeds beyond the speed of sound, a shockwave can be formed. The fast-moving object can depart the shock in the form of a pressure wave that is moving at ultrasonic frequencies. The use of this pressure wave can deliver its energy broadly into tissue of all types including plaque adhered to the vessel wall and atherosclerotic plaque accumulated in the walls of the vessel. The inclusion of raised features 300, especially those that are metal and have a narrow interface with the tissue that the wave is intending to be delivered to, can offer an advantage for delivering and amplifying the pressure wave. In some embodiments, the narrow distances and narrow contact area for the wave to propagate through can produce a physical and mechanical arc. The narrow surface contact of the raised features 300 combined with energy delivery in a pulsed fashion by the pressure waves has been observed to improve outcomes in testing. When the narrow surface of the raised features 300, above the balloon 100 where a pressure wave is propagating from contacts the diseased tissue, the interface can be in the range of 100 square nanometers to 100 square micrometers. This small surface contact area increases the focal energy in ranges of 100 times, 200 times, 500 times, 700 times, 1000 times, 1100 times, 1200 times, 1300 times, 1400 times, 1500 times, 1600 times, 1700 times, or any range of two of the foregoing values, which can be capable of enhancing the transfer of ultrasonic energy into the diseased tissue. Additionally, the small gap between the outer edge of the raised feature 300 and the diseased tissue limits the distance needed for a high power, high intensity shockwave. In some embodiments, the narrow outward facing surface of the raised features 300 can be configured to move faster than the speed of sound over a very short distance. Sometimes the short distance can be only in one direction. When these raised features 300 move at ultrasound speeds, there can be an abrupt decrease in the flow area around the raised features 300, and shock waves can be generated in the flow region between the raised feature 300 and the surface lumen that the raised features 300 are moving towards. When these raised features 300 have rough or imperfect surfaces and are moving at sonic speeds, there can be an abrupt decrease in the flow area around the raised features 300, and cavitation events can be generated in the flow region between the raised feature 300 and the surface lumen they are moving towards. These cavitation events can collapse producing shock waves in very small regions in the gap where the gap shrinks vary rapidly. Across this pulse shock wave event, the static pressure, temperature, and medium density increase almost instantaneously. The increase in focal energy acts like shock impact force that pulverizes, crushes, or fractures the tissue and/or the plaque deposits within the tissue. When a linear line of narrow contact points of the raised features 300 are aligned in a longitudinal pattern, a rapid linear expansion of the serrated line can be generated. In some embodiments, the pressure wave can be supersonic. The shock pulse can travel faster than the speed of sound.

When the pressure wave is in the subsonic range, a deflagration shock wave can be produced. The deflagration pulse shock wave can carry energy that can be absorbed by the medium carrying the pressure wave, producing effects that vary with intensity. Subsonic energy can be used to shatter or pulverize tissue. In some embodiments, the shock pulse can be subsonic. The shock pulse can travel slower than the speed of sound.

In all forms of wave energy delivery, the pressure wave can be partially absorbed by the structure of the tissue that the pressure wave contacts. Reflections at boundaries between two different tissue types or media occur because of differences in a characteristic known as the acoustic impedance Z of each substance. Impedance can be defined as Z=ρv, where ρ can be the density of the medium (in kg/m³) and v can be the speed of sound through the medium (in m/s). The units for Z can be therefore kg/(m²·s). The density of diseased versus healthy tissue can vary and so does the speed of sound through various media (including various soft tissues) and the associated acoustic impedances. For instance, the acoustic impedances for soft tissue may not vary much but there can be a big difference between the acoustic impedance of soft tissue and air and also between soft tissue and bone (or hardened plaque). In wave propagation, when the pressure wave impacts a boundary between media of different acoustic impedances, some of the pressure wave can be reflected and some can be transmitted. The greater the difference in acoustic impedance between the two media, the greater the reflection and the smaller the transmission. The impact of this difference can be significantly reduced through the inclusion of mechanical features, such as serration strips, such as strips 200 with the raised features 300, on the angioplasty balloon 100. The strips 200 with the raised features 300 can provide an impact force for the pressure wave to directly be converted from acoustic energy to impact energy increasing the effectiveness of the energy translation from the surface of the strip 200 with the raised features 300 to medium surface. This level of energy translation can reduce the need for repeated wave pulses and can be described as impedance matching. Contact energy translation of wave dynamics in combination with longitudinally oriented narrow contact points of the raised features 300 can reduce the time needed to breakup diseased tissue. Effective vessel expansion with minimal applied pressure can be envisioned.

In shockwave mechanics, the intensity reflection coefficient a can be defined as the ratio of the intensity of the reflected wave relative to the incident (transmitted) wave. This statement can be written mathematically as

$$a = \frac{{( Z 2 - Z 1 )}^{2}}{{( Z 1 + Z 2 )}^{2}} ,$$

where Z₁ and Z₂ can be the acoustic impedances of the two media making up the boundary. A reflection coefficient of zero (corresponding to total transmission and no reflection) occurs when the acoustic impedances of the two media can be the same. An impedance “match” (no reflection) provides an efficient coupling of acoustic energy from one medium to another.

The system 1000 can advantageously allow for pressure wave propagation through the cellular matrix. Wave propagation dynamics of different percussive waveforms through the contact edge of the raised features 300 through the tissue bed can be modeled to produce a radial, tangential, and/or longitudinal wave. Wave propagation dynamics of different percussive waveforms through the surface of the balloon 100 into the strips 200 with the raised features 300 and from the strips 200 with the raised features 300 through the tissue bed can be modeled to produce a radial, tangential, and/or longitudinal wave. The modeling of the pressure wave can be based on assumption of the bi-linear bit/rock interface law.

The system 1000 can advantageously allow experimental and theoretical insights. The embodiments can leverage both experimental data and theoretical models to predict and understand the mechanics of pulse wave serration angioplasty and the influence of various parameters on the effectiveness of penetration of the raised features 300 of the serrated strip 200 into tissue beds.

The system 1000 can have synergies between the strips 200 with the raised features 300 and the generator 500. In some embodiments, the generator 500 can be separate from the catheter with the balloon 100 with the strips 200 and the raised features 300. In some embodiments, the generator 500 can be integrated with the catheter with the balloon 100 with the strips 200 and the raised features 300. The generator 500 can provide energy in combination with serrations on the outer surface of the balloon 100. The generator 500 can provide various functionality such as compressing the plaque, propagating cracks, cracking the medial plaque, cracking the intimal plaque, lesion preparation, and/or breaking up the plaque. The generator 500 can produce ultrasonic vibrations or ultrasound. In some embodiments, the pressure wave can be transmitted along the longitudinally oriented structures such as the strips 200 with the raised features. The vibrational energy can be translated utilizing longitudinal, radial, or torsional oriented vibrations, or any combination therein. In some embodiments, the pressure wave can be transmitted through the medium (e.g., liquid or gas) used to fill the balloon and through the wall of the balloon 100 in addition to the plurality of strips 200 with the raised features. In some embodiments, the raised features 300 can have a shape to enable the effective transduction of the acoustic energy into the tissue. Thereby, the design of the raised features 300 can enable a more effective transmission of the acoustic energy into the diseased tissues. In some embodiments, the pressure waves can travel through a balloon inflation port to the balloon. The pressure waves can provide pneumatic and/or hydraulic energy delivery to the balloon 100. In some embodiments, the generator 500 can be provided enabling pressure waves with a series of periodic higher-pressure events followed by periodic low-pressure events. In some embodiments the high-pressure events can be 20 ATM, 15 ATM, 12 ATM, 10 ATM, 9 ATM, 8 ATM, 7 ATM, 6 ATM, or 5 ATM or some combination of these pressures. In some embodiments the low-pressure events can be 8 ATM, 7 ATM, 6 ATM, 5 ATM, 4 ATM, 3 ATM, 2 ATM, 1 ATM, or 0.5 ATM or some combination of these pressures. The range between the low-pressure event and the high-pressure event that the pressure wave produces can be 20 ATM, 19 ATM, 18 ATM, 17 ATM, 16 ATM, 15 ATM, 14 ATM, 13 ATM, 12 ATM, 11 ATM, 10 ATM, 9 ATM, 8 ATM, 7 ATM, 6 ATM, 5 ATM, 4 ATM, 3 ATM, 2 ATM, 1 ATM, or 0.5 ATM or some combination of these pressure ranges. The periodicity of the higher-pressure events and low-pressure events can be constant. The periodicity of the higher-pressure and low-pressure events can vary. The peak of the peak high pressure can be constant. The peak of the peak low pressure can be constant. The peak of the peak high pressure can vary. The peak of the peak low pressure can vary. In some embodiments, the periodicity and the level of the pressure wave between the high and low events can be constant from pulse to pulse. In some embodiments, the periodicity and the level of the pressure wave between the high and low events can vary from pulse to pulse. The generator 500 can enable fewer strips necessary to produce an effective outcome compared with systems without the generator 500. For example, two strips 200 or even a single strip 300 can produce an effective outcome compared with systems without the generator 500 with three or more strips 200. Additionally, the spaces between the raised features 300 could be made longer in system with the generator 500 while producing an effective outcome compared with systems without the generator 500. For example, the gap between adjacent raised features can be between about 2 times to about 10 times the length of the raised features. For example, a larger gap between raised features can produce an effective outcome compared with systems without the generator 500 with a shorter gap between raised features. The shape of the raised feature 300 could be made shallower or shorter in system with the generator 500 while producing an effective outcome compared with systems without the generator 500. For example, the height of the raised features can be 0.01 mm to about 0.50 mm. For example, a shorter height of the raised features can produce an effective outcome compared with systems without the generator 500 with a greater height of the raised features. The system 1000 can combine the strips 200 with the raised features 300 and the generator 500. The system 1000 can combine pressure wave delivery with serrations. In some embodiments, the complexity of the generator 500 combined with the strips 200 with the raised features 300 can be reduced compared with systems without serrations. The system 1000 can include a series pneumatic and/or hydraulic pumps to produce effective lumen gain or to break-up the disease state for some disease modalities. The energy delivery of the generator 500 and the strip 200 with the raised features 300 may have a synergistic effect. The energy delivery of the generator 500 may be more effective in combination with the nucleation sites provided by the raised features 300. The raised features 300 may increase tissue transduction of energy from the generator 500. The raised features 300 may concentrate the energy delivery of the generator 500. The raised features 300 may penetrate plaque easier in combination with energy delivery of the generator 500. The crack may propagate easier or faster with both modalities of the serrations and energy delivery of the generator 500. The crack may propagate in a more controlled manner with both modalities of the serrations and energy delivery of the generator 500. This combination of modalities of the serrations and energy delivery of the generator 500 may offer an advantage by reducing the complexity of the integrated system with the balloon 100 and the raised features 300, while minimizing the drawbacks of poor tissue transduction of the energy through the disease state and into the tissue by utilizing the raised features 300 to more effectively transmit the energy through the disease tissue state.

The pressure wave can cause crack propagation. The pressure wave can cause crack propagation along the serrations, indentations, and/or microperforations caused by the raised features 300. The pressure wave can cause crack propagation in the vessel to the medial layer. The pressure wave can create predicable and repeatable crack propagation along the serrations, indentations, and/or microperforations. The pressure wave can expand the raised features 300 in a radially outward direction that form precise indentations, cleavage lines or planes in the plaque or other locations in the vessel wall. The pressure wave can push the radially outward facing surfaces of the raised features 300 into the plaque. The radially outward facing surfaces of the raised features 300 can have small surface areas. The serrations, indentations, and/or microperforations may not be continuous when formed by the raised features 300. The serrations, indentations, and/or microperforations can disrupt the continuity of the surface of the plaque. The pressure wave can cause crack propagation along the serrations, indentations, and/or microperforations. The pressure wave can create one or more longitudinal cracks the plaque. The pressure wave can apply a tensile force by the balloon 100 on the vessel wall. The pressure wave can stretch the vessel to cause the serrations, indentations, and/or microperforations to connect forming a permanent linear dissection line. The pressure wave can cause tearing along the serrations, indentations, and/or microperforations. The pressure wave can exert a tensile force on the vessel wall to facilitate the tearing. In some embodiments, the pressure wave can only cause crack propagation. In some embodiments, the pressure wave can create serrations, indentations, and/or microperforations and cause crack propagation. In some embodiments, the pressure wave can cause both serrations and crack propagation. In some embodiments, the pressure wave can cause serrations, crack propagation, and create a shock wave. In some embodiments, the pressure wave can cause serrations, crack propagation, and lithotripsy effects.

The pressure wave can accelerate crack propagation. The pressure wave can cause pulsed movement of the strips 200 with the raised features 300. The pressure wave can cause outward movement of the strip 200 due to the pressure under the strip 200. The pressure wave can cause outward movement of the strip 200 due to the pressure in the balloon 100 under the strip 200. The pressure wave can cause the crack to quickly progress through the intima tissue. The pressure wave can cause the crack to quickly progress through the internal elastic lamina. The pressure wave can cause the crack to quickly progress through the medial tissue.

In some embodiments, shear cracking can occur at a very fast speed. In some embodiments, cracks can propagate faster than the speed of sound. In some embodiments, crack fractures can travel at speeds up to 2200 m/s. The distance to the media can be 0.1 mm so the pressure wave can cause the crack to reach the medial plane at approximately 4.5×10⁻⁸ seconds. Cracking in tightly bonded calcium molecules can slow the progression down. Calcium bonds can have a high bond energy 80 kcal/mole for a single bond and 145 kcal/mol for a double bond. This very high bond energy can slow the crack propagation.

The pressure wave can facilitate the dynamics of crack propagation. The pressure wave can create dynamic loading of the vessel. The pressure wave can facilitate longitudinal crack propagation. In some embodiments, the system 1000 can only form longitudinal cracks. The pressure wave can facilitate longitudinal crack propagation along serrations, indentations, and/or microperforations. The serrations, indentations, and/or microperforations can be linear due to the arrangement of the raised features 300 on the strip 200. The serrations, indentations, and/or microperforations can be aligned along a longitudinal axis of the vessel. In some embodiments, the cracks may not be randomly formed. In some embodiments, the crack only propagate longitudinally with the pressure wave.

In some embodiments, the system 1000 described herein can control crack propagation. The pressure wave can cause the microperforations to tear. The pressure wave can influence the propagation of the crack into deeper tissue than the raised feature 300 contacted. The raised features 300 can only produce the initial microperforations after which the raised features 300 do not contact the tissue that the raised features 300 is penetrating. The pressure wave can cause the balloon 100 in contact with the vessel wall to expand. The balloon can impart a strain on the tissue near the initial microperforations. The pressure wave can generate cleaving stress on the tissue. The pressure wave can exert a prying force on the side walls of the penetrated tissue. The pressure wave can tear the tissue along the microperforations. The pressure wave can exert strain on the vessel wall through the balloon 100. The pressure wave can magnify the strain exerted by the balloon 100. The pressure wave can facilitate crack formation. The pressure wave can facilitate crack propagation. The pressure wave can control crack propagation by preventing dissections. The pressure wave can concentrate energy delivery along the raised features 300. The concentrated delivery can reduce the formation of dissections. The pressure wave can create regularly spaced longitudinal cracks. The pressure wave can create linear incisions. The pressure wave can cause the release of strain energy that propagates along the microperforations. The strain energy can be quickly dispersed along the microperforations. The microperforations can produce the line for the strain to be released. The microperforations produce a serrated line of weakness. In some embodiments, the pressure wave can create cracks only along a straight line. In some embodiments, the pressure wave can cause the plaque to separate only along longitudinal lines. % In some embodiments, the pressure wave can form an interruption in the circumference of the vessel, thereby longitudinally segmenting the vessel. %

The systems described herein can provide several advantages. The system 1000 can have advantageous mechanisms of action. The system 1000 can provide dual-modality intravascular lithotripsy. The system 1000 can provide a serration modality that creates serrations, indentations, and/or microperforations. The serrations, indentations, and/or microperforations can cause longitudinal cracks along the axis of the vessel. The serration modality can be a mechanical modification of the plaque. The serration modality can be crack superficial plaque. The serration modality can be used independently of other modalities. The serration modality can be used in combination with other modalities. The system 1000 can provide a wave modality. The wave modality can break calcium bonds with the application of energy. The wave modality can use pressure waves to fracture calcium deposits. The wave modality can break calcium bonds in superficial plaque. The wave modality can break calcium bonds in deposits embedded within tissue. The wave modality can modify deep lesions. The wave modality can fracture calcium. The wave modality can provide acoustic energy delivery. The wave modality can provide sonic energy delivery. The wave modality can provide ultrasonic energy delivery. The wave modality can be used independently of other modalities. The serration modality can be used before the wave modality. The serration modality can be used after the wave modality. The serration modality can be used in combination with the wave modality.

The systems described herein can provide improved therapy outcomes. The system 1000 can provide concentric therapy. The system 1000 can provide pressure waves around the circumference of the vessel. The system 1000 can provide pressure waves that radiate outward. The system 1000 can provide pressure waves that extend equally around the circumference of the vessel. The system 1000 can provide pressure waves that have an outward spread. The system 1000 can provide pressure waves that transfer energy outward from the balloon 100. The pressure waves can radiate outward from the balloon 100. The system 1000 can provide therapy that originates at the same center. The system 1000 can provide eccentric therapy. The system 1000 can provide therapy that does not share the same center. The system 1000 can provide therapy that can be offset from center. The concentric therapy and eccentric therapy can be configured to treat variable vessel geometries. The concentric therapy and eccentric therapy can be configured to treat variable lesion morphologies. The pressure wave can treat all lesion morphologies. In some embodiments, the pressure wave treatment is not limited to concentric calcium. The pressure wave can treat eccentric calcium. The pressure wave can treat calcium arcs. The pressure wave can treat soft calcium. The wave modality can be concentric. The serration modality can be eccentric. The serration modality can treat different regions differently.

The systems described herein can change vessel compliance under the strain of the balloon 100. The systems described herein can change vessel compliance with a lower injury score, causing less cellular damage and lower rates of flow limiting dissections. The system 1000 can restore compliance through controlled pressure waves and serration-assisted expansion. The system 1000 can improve the ability of the vessel to expand and contract. The system 1000 can improve compliance to accommodate changes in blood pressure in volume. The system 1000 can improve tissue composition. The system 1000 can improve elasticity of the vessel by modifying embedded calcium deposits. The system 1000 can improve elasticity by forming longitudinal cracks to the medial layer. The system 1000 can improve elasticity by forming longitudinal cracks through the internal elastic lamina.

The systems described herein can allow for predicable vessel opening. The system 1000 can minimize trauma. The system 1000 can minimize vessel injury. The system 1000 can minimize dissection risk.

The systems described herein can allow for deep disruption capability. The system 1000 can provide ultrasonic energy. The ultrasonic energy can propagate deep into the vessels wall tissue for improved calcium modification. The system 1000 can create pressure waves that produce ultrasonic energy to fracture calcium. The pressure wave can pass through the calcium deposit causing internal stress in the calcium deposit. The pressure wave can induce stress to pull apart calcium bonds. The system 1000 can create micro-cracks. The system 1000 can break calcium into small fragments. The smaller fragments can remain adhered to the vessel wall. The smaller fragments can remain within the tissue.

The systems described herein can allow multiple morphology performance. The system 1000 can be effective for calcified lesions. The system 1000 can be effective for mixed-morphology lesions. The system 1000 can be effective for fibrotic lesions. The system 1000 can be effective for deep and highly calcified lesions. These lesions can be challenging to treat due to the calcium bonds. The system 1000 can modify the dense calcium bonds by fracturing. The system 1000 can be effective for superficial lesions near the lumen of the vessel. The system 1000 can be effective for deep lesions in the media. The system 1000 can be effective for deep lesions in the adventitia. The system 1000 can be effective for advance atherosclerosis. The system 1000 can be effective for treating concentric deposits, often signifying severe disease. The system 1000 can be effective for treating deposits in a nodular pattern.

The systems described herein can allow for sonic and/or ultrasonic performance. The system 1000 can allow for a predicable sonic and/or ultrasonic output. The system 1000 can include the generator 500 for predicable wave formation. The wave formation can be repeatable. The wave formation can be consistent. The wave formation can be continuous. The system 1000 can be integrated with an indeflator. The system 1000 can include an inflation device to supply the base pressure to the balloon 100. The system 1000 can include the inflation device to fill the balloon 100. The system 1000 can include the inflation device to fill the catheter 600. The system 1000 can include the inflation device to fill the reservoir. The inflation device can be connected to the connector 450. In some embodiments, the system 1000 can include a single source of fluid medium. The inflation device can be used to inflate the balloon 100 to the base pressure. In some embodiments, the inflation device does not generate sonic and/or ultrasonic pressure waves. In some embodiments, only the generator 500 generates sonic and/or ultrasonic pressure waves. The generator 500 can act on the fluid provided by the inflation device. The generator 500 can act on the fluid provides separately from the inflation device. The generator 500 can increase the pressure of the fluid in the balloon 100. The generator 500 can increase the volume of the fluid in the balloon 100. In some embodiments, the system 1000 can include two sources of fluid medium. In some embodiments, the system 1000 can include a separate source of fluid medium that fills the reservoir. The system 1000 can include an integrated inflation device. The system 1000 can provide consistent therapy delivery.

The system 1000 can be used intravascularly in various methods of use. The systems and methods described herein can provide a concentrated output. The systems and methods described herein can provide uniform energy distribution. The systems and methods described herein can be optimized to minimize energy loss. The systems and methods described herein can be optimized to maintain effective translation of energy to the lesion. The systems and methods described herein can utilize a fluid-filled system such as balloon 100 and catheter 600 to transfer energy. The systems and methods described herein can control reflection. The systems and methods described herein can control diffraction. The systems and methods described herein can maintain a constant pressure and impulse along the length of the balloon 100.

The systems and methods described herein can amplify the wave energy. The strip 200 can function as an amplifier. The raised features 300 can function as an amplifier. The design of the strip 200 and the raised features 300 can amplify and propagate sonic and/or ultrasonic pressure waves. The design of the strip 200 and the raised features 300 can amplify and propagate pressure waves across a range of lengths of the balloon 100, such as 10 mm, 16 mm, 20 mm, 40 mm, 80 mm, 120 mm, 160 mm, 200 mm, or any range of two of the foregoing values. The design of the strip 200 and the raised features 300 can amplify and propagate waves across a range of diameters of the balloon 100, such as 2.0 mm, 2.5 mm, 3.0 mm, 3.5 mm, 4.0 mm, 5.0 mm, 6.0 mm, 7.0 mm, and 8.0 mm, 9.0 mm, 10.0 mm, 11 mm, 12 mm, or any range of two of the foregoing values. The design of the strip 200 and the raised features 300 can amplify and propagate pressure waves for above the knee procedures. The design of the strip 200 and the raised features 300 can amplify and propagate pressure waves for below the knee procedures. The design of the strip 200 and the raised features 300 can amplify and propagate pressure waves for coronary procedures. The design of the strip 200 and the raised features 300 can amplify and propagate pressure waves for carotid procedures. The design of the strip 200 and the raised features 300 can amplify and propagate pressure waves for arterial-venous fistula procedures. The design of the strip 200 and the raised features 300 can amplify and propagate pressure waves for procedures in other vessels inside the vascular system. The design of the strip 200 and the raised features 300 can amplify and propagate pressure waves for procedures in other vessels outside the vascular system. The systems and methods can propagate pressure waves along the length of the catheter, which can be about 80 cm, 110 cm, 140 cm, 150 cm or any range of two of the foregoing values. The systems and methods can propagate pressure waves from the reservoir through the catheter 600 and to the balloon 100. The systems and methods can propagate waves along the length of the balloon 100. The systems and methods can propagate waves beyond the distal tip of the balloon 100.

The systems and methods described herein can channel energy. The strips 200 with the raised features 300 can channel energy into a 360° arc to restore compliance along controlled serration lines. The strips 200 with the raised features 300 can be equally spaced along the balloon 100. The balloon 100 can include one strip, two strips, three strips, four strips, five strips, six strips, or any range of two of the foregoing values. The pressure waves can propagate circumferentially. The pressure waves can improve vessel compliance.

The systems and methods described herein can have therapy benefits. The systems and methods described herein can restore natural vessel function. The systems and methods described herein can address the internal elastic lamina. The systems and methods described herein can minimize vessel recoil. The systems and methods described herein can reduce the elastic tendency of the vessel to snap back. The systems and methods described herein can produce permanent vessel remodeling with the formation of longitudinal cracks. The systems and methods described herein can produce permanent vessel remodeling with the fracturing of calcium deposits. The systems and methods described herein can promote conditions for the vessel to heal itself.

The systems and methods described herein can cause calcium disruption. The strip 200 with the raised features 300 can cause a mechanical interaction with calcium. The pressure waves can cause an ultrasonic interaction with calcium. The mechanical and ultrasonic interaction with calcium can provide a controlled massaging effect for calcium disruption. The mechanical and ultrasonic interaction can smooth the lumen. The mechanical and ultrasonic interaction can reduce turbulence. The mechanical and ultrasonic interaction can compress the plaque against the vessel wall. The mechanical and ultrasonic interaction can be a therapeutic manipulation of calcium bonds through fracturing along an ultrasonic wave. The mechanical and ultrasonic interaction can cause cyclical energy delivery. The mechanical and ultrasonic interaction can cause improved blood flow. The mechanical and ultrasonic interaction can cause vessel relaxation.

The systems and methods described herein can cause predictable lesion response. The systems and methods described herein can be compatible with varied lesion formations and depths. The systems and methods described herein can provide synergistic lesion alteration. The systems and methods described herein can alter lesions by serrations, indentations, and/or microperforations in combination with pressure wave generation. The systems described herein can alter lesions by serrations, indentations, and/or microperforations in combination with acoustic waves. The systems described herein can alter lesions by serrations, indentations, and/or microperforations in combination with fluid waves. The systems and methods described herein can reduce intimal calcification. The systems and methods described herein can reduce medial calcification. The systems and methods described herein can reduce arterial stiffness with the combination of pressure wave generation. The systems and methods described herein can increase the pathways for drug uptake with the combination of pressure wave generation.

The systems and methods described herein can provide efficient therapy. The systems and methods described herein can provide effective modification with minimal wasted energy. The systems and methods described herein can provide effective transmission of the pressure wave from the generator 500 to the balloon 100. The systems described herein can provide effective transmission of the wave through the balloon 100, the strips 200, and the raised features 300.

The systems and methods described herein can improve deliverability. The systems and methods described herein can improve the ability to cross through very narrow lesions, tortious paths, and or hardened disease. The systems and methods described herein can allow ultrasonic therapy to be delivered effectively even in distal, hard-to-reach segments. The systems and methods described herein can have features that can improve deliverability of balloon 100. The systems and methods described herein can include the strip 200 and the raised features 300. The systems and methods described herein can have improved column strength compared with other balloons. The systems and methods described herein can navigate across narrow or occluded areas in the vessel. The systems and methods described herein can have an improved stiffness to pass through the vessel.

The systems and methods described herein can support techniques to navigate vessels. The systems and methods described herein can allow for balloon inch worming. The systems and methods described herein can allow for sequentially inflating and deflating the balloon 100 to allow the balloon to inch past an occlusion, e.g., similar to the motion of an inch worm. The balloon 100 can be inflated and deflated to reach the treatment site. The balloon 100 can be inflated, the system can produce a pressure wave that emits distally pass the tip of the device. The balloon 100 can be inflated, the system can produce a pressure wave that emits distally pass the tip of the device then deflated. The systems described herein can be inflated and deflated to navigate the vessel to the treatment site. The systems described herein can be inflated and deflated for navigation before energy delivery. The systems described herein can be inflated and deflated for navigation before creating serrations, indentations, and/or microperforations.

The systems and methods described herein can be integrated with other technologies. The systems described herein can be disposable. The systems described herein can be reusable. The systems described herein can be connected to power. The systems described herein can be battery operated. The systems described herein can be compact. The systems described herein can include a generator to generate pressure waves. The systems can be handheld. The systems and methods described herein can allow for easy setup. The systems and methods described herein can allow for intuitive use. The systems and methods described herein can allow for easy workflow.

The systems and methods described herein can have a modular platform. The systems described herein can support various balloons. The systems and methods described herein can support additional future therapeutic technologies. The systems and methods described herein can support future catheter designs. The systems and methods described herein can support future balloon designs. The systems and methods described herein can support future strip designs. The systems and methods described herein can support future assembly designs.

The systems and methods described herein can allow for smart controls. The systems and methods described herein can allow for data collection. The systems and methods described herein can track when the therapy yields the calcium by measuring parameters of the vessel, including but not limited to changes in vessel diameter, vessel remodeling, changes in the uniformity of the vessel diameter across the lesion, blood pressure change proximal to the lesion, blood pressure change distal to the lesion, and changes in volumetric flow rate, or changes in acoustic Doppler flow rates or acoustic Dopler flow patterns. The systems and methods described herein can track when the therapy yields the calcium by measuring the changes in the system, including a drop in the system pressure, a change in the system high-pressure setting, a change in the systems low-pressure setting, a change in the pressure range setting. The systems and methods described herein can allow for tunability to lesion presentation. The systems and methods described herein can allow for variation in maximum pressure by controls. The systems and methods described herein can allow for variation in frequency by smart controls. The systems and methods described herein can allow for variation in parameters of energy delivery by smart controls.

The systems described herein can have heuristic smart capabilities for consistent outcomes. The heuristic smart capabilities can focus on key cues to make decisions. The heuristic smart capabilities can focus on certain parameters within the system such as pressure, frequency, and/or amplitude. The heuristic smart capabilities can include certain parameters outside of the system such as changes in blood pressure (near the treatment zone and/or taken in specific regions such as ankle and/or brachial blood pressures), changes in blood flow, angiographic changes in the balloon shape, changes in the lesion diameter, changes in the overall average lumen diameter across the lesion, changes in volumetric blood flow, and/or changes in acoustic Dopler flow patterns. The heuristic smart capabilities can ignore irrelevant data. The heuristic smart capabilities can be AI-driven. The heuristic smart capabilities can be provided with data from sensors within the system. The heuristic smart capabilities can improve precision in delivering the pressure waves. The systems described herein can include indeflator integration. The systems described herein can allow seamless control of balloon inflation with synchronized energy delivery.

The systems described herein can include data transmission. The systems described herein can include a Bluetooth interface to communicate data from the generator 800 to an external device. The external device can be any external device, such as an app on a phone, a smartphone, a computer, a tablet, and/or a smartwatch. The systems can include Bluetooth data transmission into the generator 800. The systems can include data transmission for system updates. The systems can include Bluetooth data transmission out of generator 800. The systems can include Bluetooth data transmission both into and out of the generator 800. The systems can include any means of data transmission. In some embodiments, the system can include transmission in only one direction, such as out of the generator for collecting data such as the number of pulses, the pressure of the system and/or at which pulses did the pressure change up or down.

The systems described herein can have features to improve predictability. The systems described herein can produce pressure waves with certain characteristics. The systems and methods described herein can have a controlled frequency within a narrow range of frequencies, such as 1 Hz, 2 Hz, and/or 3 Hz for pressure wave generation. The systems and methods described herein can have a controlled pressure within a narrow range of pressure, such as a peak positive pressure within the system of 8 ATM, 9 ATM, 10 ATM, 11 ATM, 12 ATM, 13 ATM, 14 ATM, and/or 15 ATM. The systems and methods described herein can have controlled pressure wave generation with pressure waves having consistent properties. The systems and methods described herein can produce pressure waves with a specific acoustic and ultrasonic frequency signature. The systems and methods described herein can produce pressure waves with a specific peak frequency. The systems and methods described herein can minimize vessel injury. The systems and methods described herein can allow controlled crack propagation. The systems and methods described herein can allow controlled energy delivery. The systems and methods described herein can allow delivery of energy that passes through soft tissue. The systems and methods described herein can allow for low pressure angioplasty. The systems and methods described herein can allow for controlled serration and sonic output that reduces uncontrolled dissections. In some embodiments, the systems and methods described herein can allow for controlled crack propagation only along longitudinal lines. In some embodiments, the systems and methods described herein can allow for controlled longitudinal segmentation of the vessel along the length of balloon 100. These and other advantages will be further described herein.

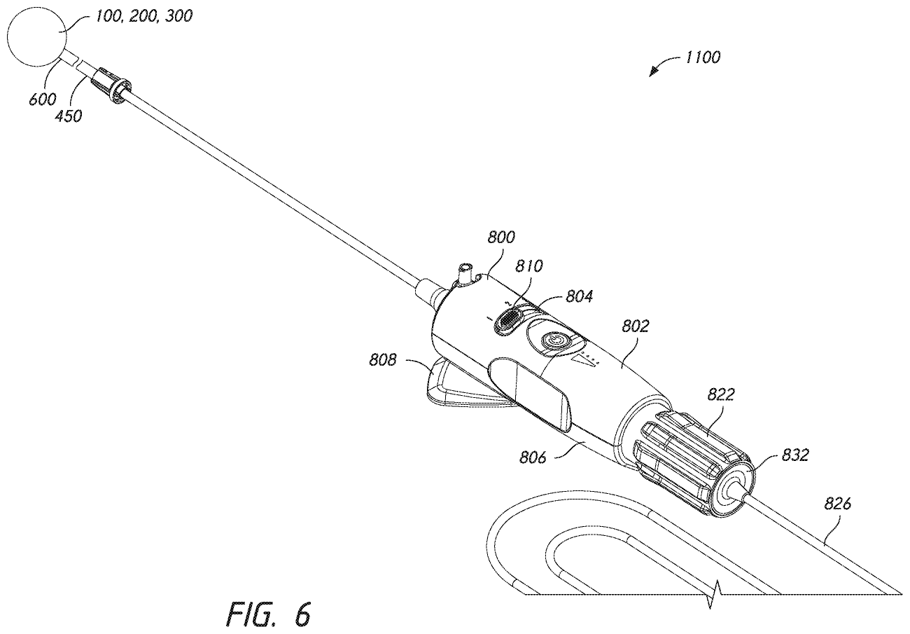

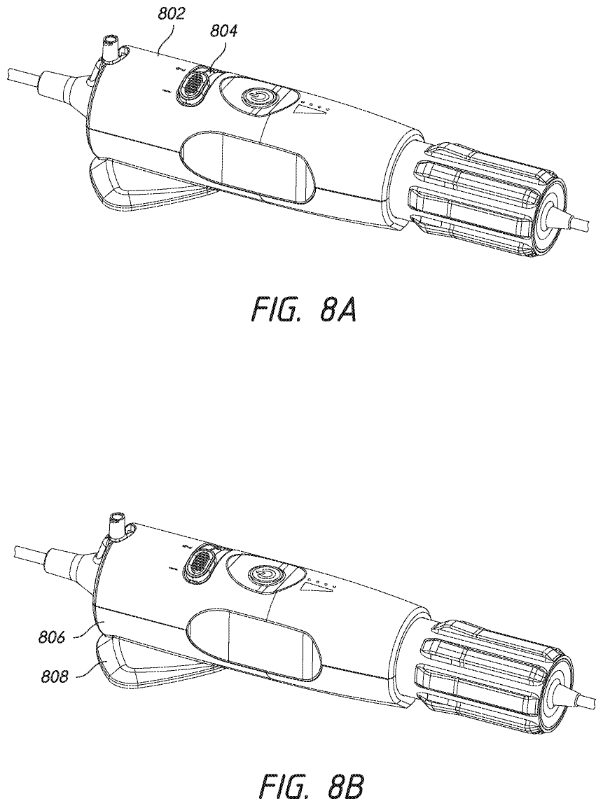

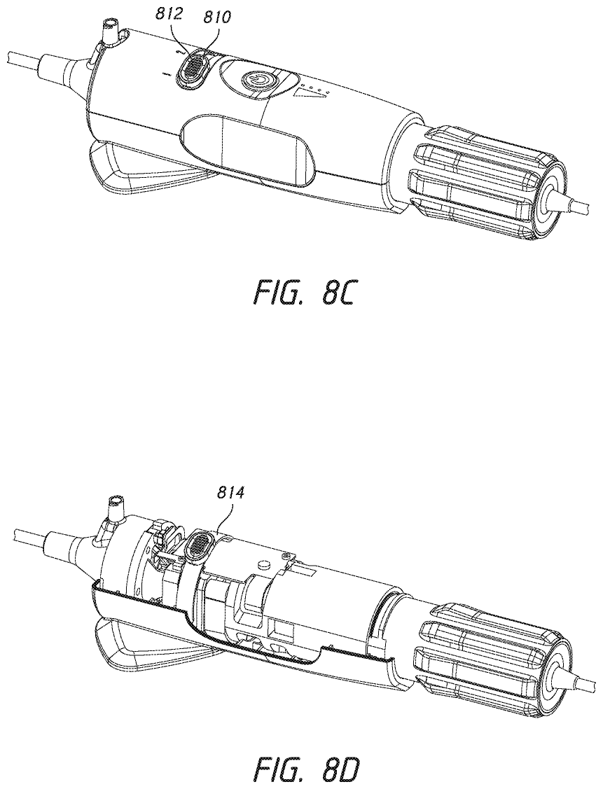

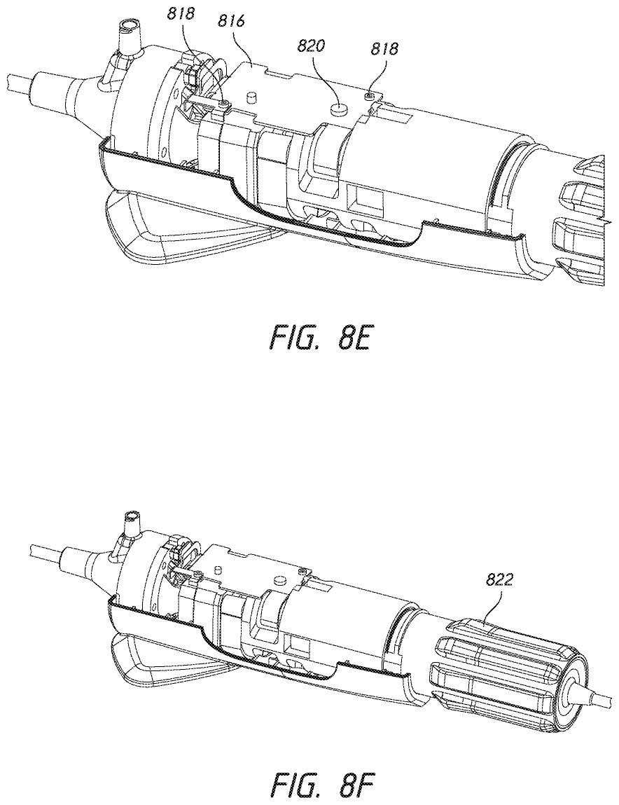

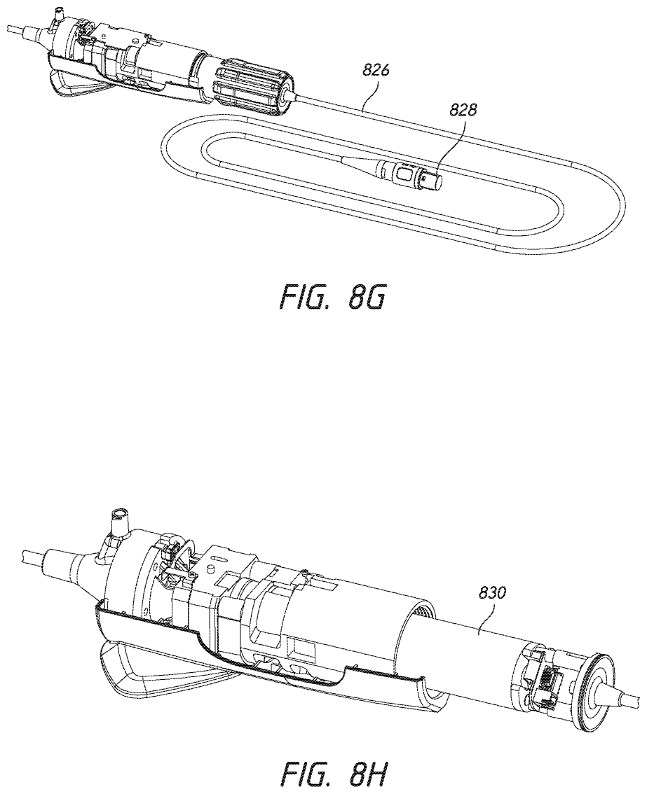

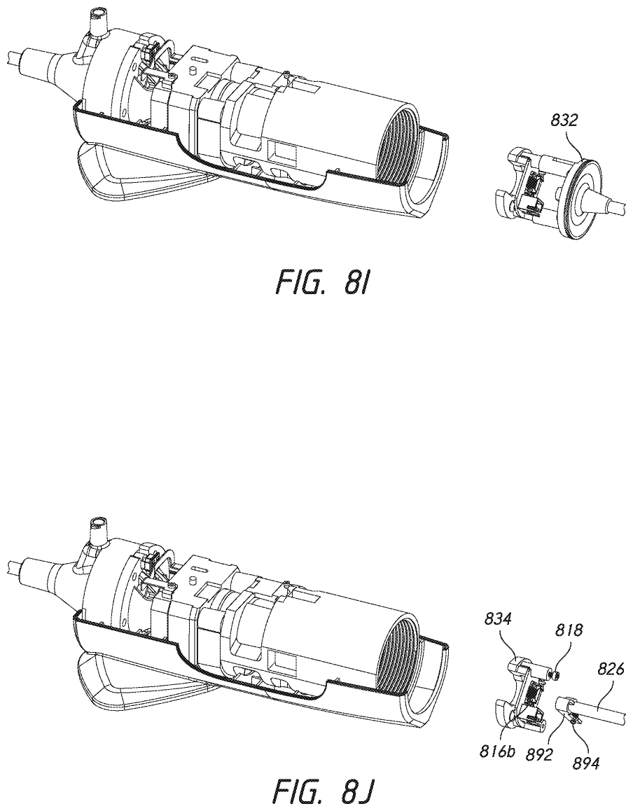

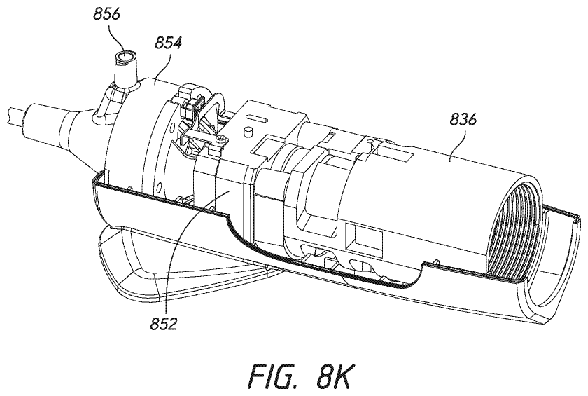

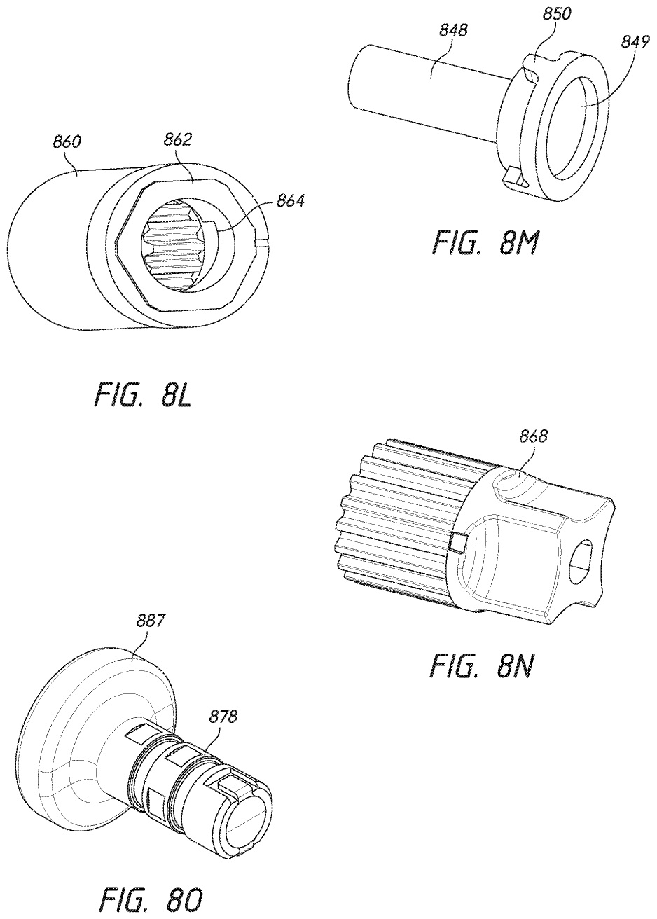

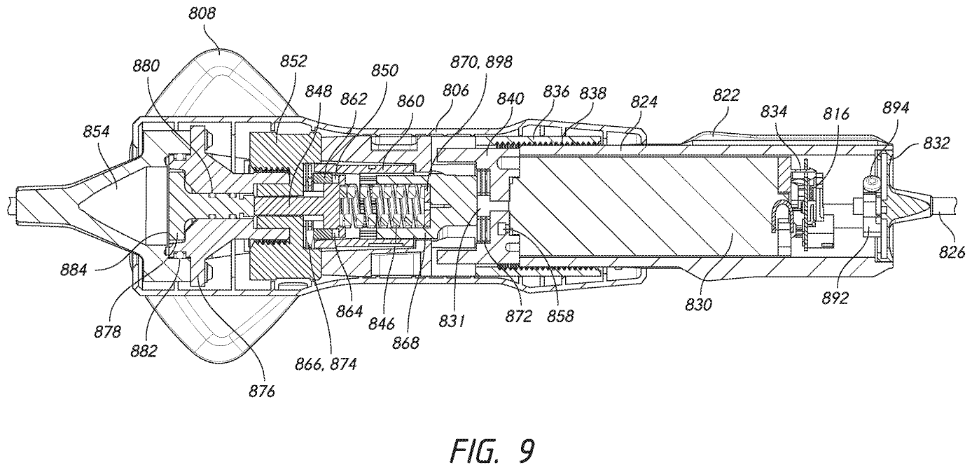

FIGS. 6-11 illustrate an embodiment of a pulse enhanced balloon catheter system 1100. The system 1100 can include any of the features of the system 1000 described herein. The system 1100 can provide a relatively large axial coverage of lesions. The system 1100 can provide lithotripsy along the entire length of the strips 200 with the raised features. The system 1100 can provide lithotripsy along the entire length of the balloon 100. The system 1100 can provide lithotripsy beyond the length of the balloon 100. The system 1100 can provide lithotripsy along the entire circumference of the ballon 100. The system 1100 can provide lithotripsy through the lobes 120 of the balloon 100. The system 1100 can provide lithotripsy through the strips 200 with the raised features 300. The system 1100 can be effect for 360 degree treatment. The system 1100 can provide treatment for full-circle calcium. The system 1100 can provide treatment regardless of the arc of the calcium. The system 1100 can include the balloon 100 described herein. The system 1100 can include one or more strips 200 described herein. The system 1100 can include raised features 300 as described herein. The system 1100 can include an generator 800. The generator 800 can include any feature or function of the inflation device 400 described herein. The generator 800 can include any feature or function of the generator 500 described herein. The system 1100 can be a variation of the system 1100 with the generator 800 to generate pressure waves as described herein. The generator 800 can utilize a motor driven system to generate pressure waves. The generator 800 can utilize a mechanical piston and cam system to repeatably strike a membrane to generate the pressure waves. The generator 800 can include a reservoir 854 for fluid. The generator 800 can include a membrane 884, a cam hammer 848, a piston 878, and a spring 846. In some embodiments, translating the membrane 884 can contribute to generating the pressure wave. In some embodiments, translating the cam hammer 848 can contribute to generating the pressure wave. In some embodiments, translating the piston 878 can contribute to generating the pressure wave. In some embodiments, compressing the spring 846 can contribute to generating the pressure wave. The generator 800 can produce pressure waves using these components, as described herein. The system 1100 can include any feature or function of the catheter 600 described herein. The catheter 600 can connect the generator 800 to the balloon 100 with strips 200 and raised features 300. The system 1100 can have any feature described herein. The system 1100 can perform any function described herein. The system 1100 can include the connector 450. The connector 450 can include a stopcock. The connector 450 can be a uniquely designed connector that limits the use of the generator with catheters not designed for use with the generator. The connector 450 can be a multiport stopcock. The connector 450 can be a three-way stopcock. The connector 450 can be a four-way stopcock. The connector 450 can be a multiport manifold. The connector 450 can be a three-way manifold. The connector 450 can be a four-way manifold. The connector 450 can be connected and disconnected from the catheter 600 and the balloon 100 with strips 200 and raised features 300. The connector 450 can be connected and disconnected from the generator 800. The connector 450 can be connected and disconnected from the inflation device 400. The connector 450 can manage fluid. The connector 450 can ensure a closed-system design. The connector 450 can allow for multi-directional flow. The connector 450 can allow for inflation device 400 to be isolated from the generator 500 pressure wave and the catheter 600. The inflation device 400 can allow the system 1100 to be filled. The balloon 100 can be filled to be in contact with the vessel wall in some methods. The fluid can be maintained at the same pressure, such as the base pressure, as described herein. The fluid can be within the balloon 100, the catheter 600, and the reservoir 854 of the generator 800 described herein. The system 1100 can be filled with fluid from the inflation device 400, such as indeflator. There can be a one-way valve between the inflation device 400 can the catheter 600 and balloon 100. There can be a four-way valve between the inflation device 400, the generator 500, the catheter 600, and balloon 100.

The system 1100 can be configured to produce the pressure wave. In some embodiments, the base pressure of the balloon 100 can be set prior to the generator 800 being actuated. The base pressure can be 0.5 atm, 1 atm, 2 atm, 3 atm, 4 atm, 5 atm, 6 atm, 7 atm, 8 atm, 9 atm, 10 atm, 11 atm, 12 atm, between 1 atm and 6 atm, between 0.5 atm and 12 atm, or any range of two of the foregoing values. The fluid in the reservoir 854 can be at the base pressure. The fluid in the catheter 600 can be at the base pressure. The fluid in the balloon 100 can be at the base pressure. The fluid that fills the fluidic connections of the system 1100 can be at the base pressure. The base pressure can press against one side of the membrane 884. The base pressure can push the membrane 884 back to the original starting position. The base pressure of the reservoir 854 can maintain the membrane 884 in a neutral position. The base pressure of the reservoir 854 can maintain the spring 846 in a neutral position.

The system can maintain the pressure of the balloon 100 below a threshold value throughout the procedure. The threshold value can be 8 atm, 9 atm, 10 atm, 11 atm, 12 atm, 13 atm, 14 atm, 15 atm, 16 atm, 17 atm, 18 atm, 19 atm, 20 atm, or any range of two of the foregoing values. The threshold value can be lower than typically pressure for balloon angioplasty. The balloon 100 can be inflated to the base pressure during the procedure to contact the vessel wall. The fluid used to fill the balloon can maintain the base pressure during the procedure as pressure waves are generated. The pressure wave can produce an acute rapid rise of pressure in the balloon 100 as the acoustic wave passes through it. The system 1100 can be configured to self limit the high-pressure threshold during the acute rapid pressure rise. The self-limiting design can be driven by the equilibrium state of the forward piston impact of the piston 878 on the membrane 884 against the back pressure of the fluid in the system 1100. As the pressure on the fluid side of the membrane 884 rises, the membrane movement due to the impact of the piston 878 is reduced. At the threshold pressure value, the piston impact no longer is able to displace the membrane 884 into the fluid reservoir 854. This designed self-limiting threshold can be used to limit physician error in over pressurizing the balloon 100 causing balloon failure or rupture.

The system 1100 can be configured to spike the pressure. In some embodiments, the pressure of the balloon 100 can spike when the generator 800 is actuated. The maximum pressure of the spike can be 8 atm, 9 atm, 10 atm, 11 atm, 12 atm, 13 atm, 14 atm, 15 atm, 16 atm, 17 atm, 18 atm, 19 atm, 20 atm, 21 atm, 22 atm, 23 atm, 24 atm, 25 atm, 26 atm, 27 atm, 28 atm, 29 atm, 30 atm, between 8 atm and 10 atm, or any range of two of the foregoing values. The pressure of the fluid in the reservoir 854 can spike. The pressure of the fluid in the catheter 600 can spike. The pressure of the fluid in the balloon 100 can spike. The pressure of the fluid of the system 1100 can spike. The pressure of the fluid can spike when the membrane 884 is deflected. The pressure of the fluid can spike when the membrane 884 is moved from the original starting position. The pressure of the fluid can spike when the membrane 884 deflects from the neutral position. The pressure of the fluid can spike when the membrane 884 moves forward. The pressure of the fluid can spike when the piston 878 is moved from the original starting position. The pressure of the fluid can spike when the piston 878 is struck by the cam hammer 848. The pressure of the fluid can spike when the piston 878 moves forward. The pressure of the fluid can spike when the piston 878 strikes the membrane 884. The pressure of the fluid can spike when the cam hammer 848 is moved from the original starting position. The pressure of the fluid can spike when the cam hammer 848 strikes the piston 878. The pressure spike can be nearly instantaneous after the cam hammer 848 is actuated. The pressure of the fluid can spike when the spring 846 is unloaded. The pressure of the fluid can spike when the spring 846 imparts a greater than baseline force on the cam hammer 848. The pressure of the fluid can spike when the membrane 884 deflects a volume of fluid. The pressure of the fluid can spike when the membrane 884 changes the pressure of fluid in the system 100 when actuated by the cam hammer 848 and the piston 878.

The generator 800 can function as a pressure wave source. The generator 800 can include the membrane 884, the cam hammer 848, the piston 878, and the spring 846, among other features described herein. The generator 800 can be within the proximal handle of the system 1100. The generator 800 can be outside of the balloon 100. The generator 800 can produce a pressure wave through movement of the cam hammer 848, the piston 878, and the spring 846. The cam hammer 848 can impact the piston 878 that impacts the membrane 884. The membrane 884 can be deflected to change the pressure of the fluid within the reservoir 854. The membrane 884 can be deflected to change the pressure of the fluid in the catheter 600. The membrane 884 can be deflected to change the pressure of the fluid within the balloon 100. The system 1100 can include a single actuator to create pressure waves. The cam hammer 848 can modulate the pressure of the balloon 100 from a baseline pressure. The cam hammer 848 can produce a series of pressure impulses. The cam hammer 848 can modulate pressure with a waveform. The cam hammer 848 can move at a very fast speed that produces a pressure wave with an ultrasonic waveform. The periodicity of the cam hammer 848 can have a frequency less than 0.5 Hz, 0.5 Hz, greater than 0.5 Hz, less than 1 Hz, 1 Hz, greater than 1 Hz, less than 2 Hz, 2 Hz, greater than 2 Hz, less than 3 Hz, 3 Hz, greater than 3 Hz, less than 4 Hz, 4 Hz, greater than 4 Hz, less than 5 Hz, 5 Hz, greater than 5 Hz, or any range of two of the foregoing values. The cam hammer 848 can vary the pressure to a resonant frequency of the balloon 100.

The system 1100 can include the inflation device 400. The inflation device 400 can provide the base pressure. The inflation device 400 can include a source of fluid to inflate the balloon 100. The inflation device 400 can be connected to the reservoir 854. The balloon 100 can be inflated by the inflation device through the reservoir 854. The reservoir 854 can include the connector 856. The connector 856 can allow the balloon 100 to be inflated. The connector 856 can receive a source of fluid to inflate the balloon 100. The inflation device 400 can be in fluid communication with the fluid in the reservoir 854. The balloon 100 can be at least partially inflated by the inflation device 400. The balloon 100 can be inflated from lobes 120 first. The lobes 120 can be expanded. The lobes 120 can experience minimal resistance to filling. The lobes 120 can apply a pressure to the inside wall of the vessel 10. The lobes 120 can apply a pressure to the strip 200. The lobes 120 can apply a pressure to the inside wall of the strip 200. The lobes 120 can apply a pressure to rotate the strip 200. The strip 200 can be perpendicular to the surface of the ballon 100. The lobes 120 can apply pressure to orient the strip 200 perpendicular to the surface of the balloon 100. In some methods, the strips 200 can apply a pressure to the inside wall of the vessel 10 when the balloon 100 is at least partially inflated. In some methods, the strips 200 can produce microperforations when the balloon 100 is at least partially inflated. The balloon 100 can be fully inflated by the inflation source 400. The balloon 100 can apply the base pressure to the vessel wall. The balloon 100 can be in contact with the vessel wall at the base pressure. The inflation source 400 can be connected to the connector 856 of the reservoir 854. The inflation source can be at another location along the system 1100. In some embodiments, the inflation device 400 is separate from the generator 800. In some embodiments, the inflation device 400 is integrated with the generator 800.

The system 1100 can include the generator 800 to produce the pressure wave within the reservoir 854. The system 1100 can have a single pressure wave source. The generator 800 can act on the fluid in the reservoir 854. The inflation device 400 can be the inflation source. In some embodiments, the inflation source and the pressure wave source can be fluidically coupled to the balloon 100 in parallel. In some embodiments, the inflation source and the pressure wave source can be fluidically coupled to the balloon 100 in series. In some embodiments, the inflation source and the pressure wave source can both act on fluid in the reservoir 854. In some embodiments, the inflation source and the pressure wave source can be fluidically coupled with the reservoir 854. In some embodiments, the inflation source and the pressure wave source can change the pressure of the balloon 100 from the proximal end of the balloon 100.

The pressure wave source can create the pressure wave. The pressure wave can have any parameter described herein. The pressure wave can be a multi-component wave. The pressure wave can include an ultrasonic wave frequency. The pressure wave can include an infrasonic wave frequency. The pressure wave can include a subsonic wave frequency. The pressure wave can include an acoustic wave frequency. The pressure wave can include a combination including an ultrasonic wave frequency and one or more additional frequencies. The pressure wave can include a combination including an ultrasonic wave frequency and an acoustic wave frequency. In some embodiments, the pressure wave comprises frequency in both the acoustic and the ultrasound spectrums. In some embodiments, the primary frequency of the pressure wave can be in the ultrasonic frequency range. The pressure wave can include any combination of waves described herein. The pressure wave can have wave components that travel at different speeds. The pressure wave can have more than a single frequency carried on the wave. The pressure wave can include a set of frequencies that are carried with a calculated average or center frequency. In some embodiments, the calculated average frequency comprises ultrasonic frequency. The pressure wave can travel through liquid medium. The pressure wave can travel through the liquid medium used to fill the balloon 100. The pressure wave can carry one or more frequencies in a wave signal. The wave signal can be a time-varying parameter that oscillates periodically. The wave signal can carry a variation in amplitude, frequency, and/or phase. The waveform can describe the shape of the wave signal over time. The waveform can have any shapes described herein. The wave signals can be a time-varying quantity carried by the pressure wave. The pressure wave can be frequently modulate to carry the wave signal. The wave signals can travel through the balloon 100 and into tissue. The wave signals can travel through the strips 200 and raised features 300 and into tissue. The wave signals can transfer from medium to medium. The wave signals can include a time-varying acoustic frequency. The wave signals can include a time-varying ultrasonic frequency. The wave signals can travel through solid objects such as the strips 200, the raised features 300 and the wall of the balloon 100. The pressure wave can include an explosive acoustic wave or shock wave that carries the wave signal. The pressure wave can travel through solid objects such as the strips 200, the raised features 300 and the wall of the balloon 100. The pressure wave can have components that travel through solid objects. The pressure wave can have at least one wave signal that travels at the speed of sound.

The pressure wave can pressurize and depressurize the liquid medium of the balloon 100. The pressure wave can pressurize and depressurize the liquid medium that the pressure wave travels through. The pressure wave can be a disturbance that travels through the liquid medium. The pressure wave can be a non-linear pressure wave. The pressure wave can travel through the liquid medium of the balloon 100. The pressure wave can be a fluid wave. The pressure wave can be a shock wave. The pressure wave can be an acoustic shock wave. The shock wave can be a pressure wave with a specific speed and intensity. The shock wave can have a sudden, steep increase in pressure. The shock wave can propagate faster than the speed of sound. The shock wave can propagate with supersonic speed. The shock wave can concentrate energy in a region along the length of the balloon 100. The shock wave can propagate outward through the balloon 100 into the vessel. The shock wave can propagate distally through the balloon 100 into the vessel. The shock wave can propagate through the strips 200. The shock wave can propagate through the raised features 300. The pressure wave can be an acoustic wave.

The strips 200 and the raised features 300 can interact with the pressure wave. The strips 200 and the raised features 300 can have a rising and falling from as the pressure wave travels through the liquid medium of the balloon 100. The strips 200 and the raised features 300 can move under the influence of the fluid wave. The strips 200 and the raised features 300 can direct energy from the pressure wave. The raised features 300 can concentrate the mechanical force of the pressure wave by increasing the mechanical force several-fold. The raised features 300 can focus the energy of the pressure wave from the large surface area of the balloon 100 to the smaller surface area of the raised features 300. The pressure wave can propagate radially outward from the strip 200 to the raised features 300. The smaller surface area of the raised features 300 relative to the strip 200 can concentrate energy from the pressure wave. The raised features 300 can direct the pressure wave. The raised features 300 can direct the acoustic wave. The pressure wave can propagate along the edge of the raised features 300. The pressure wave can propagate longitudinally along the raised features 300. The pressure wave can be caused by a change in pressure within the balloon 100. The pressure wave can be caused by volumetric change in fluid of the balloon 100. The pressure wave can be acoustically transmitted. The pressure wave can be transmitted through the strips 200. The pressure wave can be transmittal distal to the balloon 100. The pressure wave can be transmitted along the length of the balloon 100. The pressure wave can be transmitted from the balloon 100 to the strip 200. In some embodiments, a portion of the pressure wave can be reflected when transmitted to the strip 200. The pressure wave can be transmitted from the strip 200 to the raised features 300. The pressure wave can be transmitted from the balloon 100 to the tissue. In some embodiments, a portion of the pressure wave can be reflected when transmitted to the tissue. The pressure wave can be transmitted from the strip 200 to the tissue. The pressure wave can be transmitted from the raised feature 300 to the tissue. In some embodiments, a portion of the pressure wave can be reflected when transmitted to the tissue.

The strip 200 and the raised features 300 can comprise a material different than the balloon 100. The strip 200 and the raised features 300 can comprise a material that is more reflective of the ultrasonic signal than the balloon 100. There can be an acoustic impedance mismatch as described herein. The materials of the balloon 100, the strips 200, and the raised features 300 can be selected to capitalize on this concept. The material of the strips 200 and the raised features 300 can be selected to be more dense than the calcium to deliver most of the acoustic energy traveling through the strips 200 and the raised features 300 to the calcium. The shape of the strips 200 and the raised features 300 can be selected to further enhance energy delivery. The angle of incidence between the raised features 300 and the calcium can produce shear waves in the calcium. The raised features can have an arced interfaces to transmit the acoustic energy through multiple angles of incidence. This can produce a shearing that more effectively cracks calcium. The raised features can have sharp edged interfaces to transmit the acoustic energy through narrow contact zones. Surface features or surface roughness of the contact zone can create narrow acoustic energy transmission zones. Narrow contact zones can produce a more precise region for the energy to travel into the calcium, increasing the regional shearing, more effectively cracking the calcium. In some embodiments the raised features 300 can amplify the stress. Bearing pressure is the case where one rigid or elastic body contacts a separate rigid or elastic body. Bearing pressure is determined by the opposing normal force between the bodies divided by the contact area between the bodies. “Point Force” means an intentional bearing contact area reduction, which results in increase in bearing pressure which is inversely proportional to the contact area reduction. Assuming the entire pressure wave effects the most proximal single raised feature 300, the raised feature 300 will exerts a mechanical advantage, amplifying the force of the pressure wave. The magnification can be approximately equal to the height divided by the width of the raised features 300, or 0.01 mm divided by 0.0002 mm or 50 times. The strip 200 and the raised features 300 can have a magnifying effect. The raised features 300 can amplify the effect of the pressure wave into the tissue. The strip 200 and the raised features 300 can have a narrow surface area. The strip 200 and the raised features 300 can amplify the pressure wave. The strip 200 and the raised features 300 can amplify the mechanical force of the pressure wave. The strip 200 and the raised features 300 can amplify the mechanical force of the pressure wave by 50 times. The raised features 300 can amplify the mechanical force of the pressure wave by 10 times, 20 times, 30 times, 40 times, 50 times, 60 times, 70 times 100 times, or any range of two of the foregoing values The strip 200 and the raised features 300 can amplify the pressure wave 10×, 15×, 20×, 25×, 35×, 50×, less than 100×, 100×, greater than 100×, 1000×, greater than 1000×, 1100×, greater than 1100×, 1200×, greater than 1200×, 1300×, greater than 1300×, 1400×, greater than 1400×, 1500×, greater than 1500×, or any range of two of the foregoing values. The lithotripsy effect of the pressure wave to break calcium can be amplified by the raised features 300. The raised features 300 can be shaped for treatment of peripheral artery disease. In some embodiments, the raised features 300 can distribute a point force compared with the pressure on the area of the strip 200. The raised features 300 can concentrate the pressure by 10×, 15×, 20×, 25×, 35×, 50×, less than 100×, 100×, greater than 100×, 1000×, greater than 1000×, 1100×, greater than 1100×, 1200×, greater than 1200×, 1300×, greater than 1300×, 1400×, greater than 1400×, 1500×, greater than 1500×, or any range of two of the foregoing values. The raised features 300 can deliver 1000× focal pressure. The raised features 300 can crack plaque. The raised features 300 can release the internal elastic lamina.

The pressure wave can travel along the length of the balloon 100. The pressure wave can impact the raised features 300 at different times. The pressure wave can come in contact with each raised features 300 as the pressure wave propagates distally. The pressure wave can impact each individual raised feature 300 at a given time. Therefore the pressure of the entire pressure wave can be magnified to a single raised feature 300. Assuming the entire pressure wave exerts the pressure amplification at a single raised feature 300 having a projected surface area of 0.00226 mm², then the magnification of the pressure wave can be 41,738 times. The strip 200 and the raised features 300 can amplify the pressure wave. In some embodiments, the raised features 300 can amplify the pressure waves when all of the pressure is concentrated to a single raised feature 300. For example, the pressure wave can be magnified to a single raised feature. The strip 200 and the raised features 300 can amplify the pressure wave 10,000×, greater than 10,000×, 20,000×, greater than 20,000×, 30,000×, greater than 30,000×, 40,000×, greater than 40,000×, 50,000×, greater than 50,000×, 60,000×, greater than 60,000×, 70,000×, greater than 70,000×, 80,000×, greater than 80,000×, 90,000×, greater than 90,000×, 100,000×, greater than 100,000×, 200,000×, greater than 200,000×, 300,000×, greater than 300,000×, 400,000×, greater than 400,000×, or any range of two of the foregoing values. The raised features 300 innovatively amplify the pressure wave based on the movement of the fluid along a wave.

The pressure wave can travel within the balloon 100 along a direction. The pressure wave can originate proximal to the proximal end of the balloon 100. The system 1100 can form a pressure wave that propagates within the balloon 100 along the length of the balloon 100. The pressure wave can enter the proximal end of the balloon 100. The pressure wave can travel along the length of the balloon 100 from the proximal end of the balloon 100 to the distal end of the balloon 100. The pressure wave can travel distally from the distal end of the balloon 100. The system 1100 can form a pressure wave that propagates distally. In some embodiments, the system 1100 generates the pressure wave with the generator 800. The generator 800 can include the membrane 884 and features to strike the membrane 884. The generator 800 can generate the pressure wave from a location outside of the balloon 100. The location outside of the balloon 100 facilitates the directional movement of the pressure wave.

In some embodiments, the system 1100 does not include features within the balloon 100 that generate the pressure wave. In some embodiments, the system 1100 does not include an arc generator. In some embodiments, the system 1100 does not include features within the balloon that generate shockwaves by discharging energy. These features can produce an effect that emanates as a burst from the energy source. These features can produce inconsistent results along the length of the balloon. In contrast, the generator 800 of the system 1100 can produce an effect that emanates from the generator 800. The generator 800 of the system 1100 can produce more consistent results along the length of the balloon.

The system 1100 can be adjusted to change parameters of the pressure wave. The system 1100 can include the spring 846 for increasing or decreasing the striking force of the components that strike the membrane 884. The system 1100 can include the spring 846 for increasing or decreasing the amplitude of the pressure wave. The amplitude of the pressure wave can be adjustable by adjusting the compression of the spring 846. The system 1100 can include the adjuster 822 for changing the compression of the spring 846 to change the pressure generated. The system 1100 can include the cam hammer 848 for generating the pressure waves by impacting the piston 878 which impacts the membrane 884. The system 1100 can include the motor 830 for determining the speed of the pressure waves. The spring 846 can be expanded and compressed as the cam can 860 rotates. The spring 846 can be expanded and compressed as the motor 830 rotates The spring 846 can be expanded and compressed as the motor shaft 831 of the motor 830 rotates. The spring 846 can be expanded and compressed as the adjuster translates. The spring 846 can be expanded and compressed as the adjuster 822 rotates. The compression of the spring 846 can be determined by the position of the adjuster 822. The adjuster 822 can be move toward the reservoir 854 to increase the compression of the spring 846. The adjuster 822 can be move away from the reservoir 854 to decrease the compression of the spring 846. The compression of the spring 846 can be determined by the position of the motor 830. The system 1100 can be a spring based system. The compression of the spring 846 can change the amplitude of the pressure wave. The compression of the spring 846 can change the striking force of the piston 878 on the membrane 884.

The system 1100 can create a shock pulse. The cam hammer 848 can be tensioned by the spring 846. In some embodiments, the spring 846 can be a compression spring as shown in the illustrated embodiment. The spring 846 can store energy when compressed. In some embodiments, the spring 846 can be a compression spring. The spring 846 can store energy when compressed. The spring 846 can release energy by returning to the uncompressed length. The spring 846 can release stored potential energy. The spring 846 can release energy when an external compressive force is removed. The spring 846 can convert potential energy into kinetic energy. The spring 846 can be loaded and unloaded during movement of the cam hammer 848. The system can allow for the loading and unloading of the spring 846 as the cam hammer 848 is rotated. The degree of compression or tension of the spring 846 can determine the amplitude of the pressure wave. The greater compression of the spring 846 can cause a higher amplitude of the pressure wave. The lesser compression of the spring 846 can cause a lower amplitude of the pressure wave. The system 1100 can allow for the near instantaneous delivery of pressure waves to the balloon 100. The cam hammer 848 can be located outside of the body of the balloon 100. The cam hammer 848 can be located at any position proximal to the balloon 100. In some embodiments, the system 1100 does not include pulse generators within the body of the balloon. The cam hammer 848 can produce the pressure wave when the membrane 884 is struck by the piston 878. The pressure wave can propagate from the reservoir 854 to the balloon 100. The pressure wave can propagate along the length of the balloon 100 from the proximal end to the distal end. In some embodiments, the pressure wave does not dissipate along the length of the balloon 100. In some embodiments, the pressure wave can travel the length of the balloon 100 with the same or substantially same wave signal or waveform. In some embodiments, there can be minimal attenuation over the length of the balloon 100. In some embodiments, there can be minimal dispersion over the length of the balloon 100. In some embodiments, the pressure wave can travel the length of the balloon 100 with the same frequency profile. In some embodiments, the pressure wave can travel the length of the balloon 100 without interruption. In some embodiments, the pressure wave can travel the length of the balloon 100 undisturbed. In some embodiments, the pressure wave is equally distributed along the length of the balloon 100 over time. In some embodiments, the pressure wave may not be concentrated at specific locations within the body of the balloon 100, such as found in arc generators and other features within balloon designs that generate shockwaves. In some embodiments, the pressure wave may not originate at specific locations within the body of the balloon 100. In some embodiments, the pressure wave originates outside of the body of the balloon 100.

The system 1100 can create pressure waves for lithotripsy. Lithotripsy can be used to break calcium bonds in vivo. Lithotripsy can be used to break plaque into small fragments. The system 1100 can create a pressure wave. The pressure wave can be a shock wave. The pressure wave can be a compression wave generated by an impact. The pressure wave can be generated by one or more percussion components that can move forward and backward. The percussion component can be the membrane 884. The percussion component can be the piston 878. The percussion component can include the cam hammer 848. The percussion component can move forward and backward as a motor shaft 831 of a motor 830 rotates. The cam hammer 848 can move forward and backward to strike the piston 878. The piston 878 can transfer the impact force onto the membrane 884. The membrane 884 can transfer the impact force to the adjoining liquid medium in the reservoir 854. The pressure wave can be generated by the percussion component that is accelerated by the motor 830 at a speed of 1 Hz, 2 Hz, or 3 Hz or higher speeds. The system 1100 can produce periodic pressure waves that in combination with the serration angioplasty balloon produce a impact energy for lithotripsy.

The generator 800 can produce the pressure waves for the synergistic effect with serrations. The generator 800 can generate and direct pressure waves into fluid of the attached catheter 600. The generator 800 can have several advantageous features described herein. The generator 800 can allow the user to adjust various parameters of the pressure wave. The generator 800 can provide adjustable frequency. The generator 800 can provide adjustable wave power. The generator 800 can provide adjustable wave intensity. The parameters of the generator 800 can be tunable. The frequency and intensity of the pressure waves can be tunable. The operator can customize the frequency and intensity by lesion type. . . . The operator can customize the frequency and intensity by anatomy type. The generator 800 can provide adjustable baseline pressure. The generator 800 can include a portable, hand-held housing. The generator 800 can provide ergonomic features. The generator 800 can prevent physical access to internal components and connectors. The generator 800 can insulate user from heat, acoustic sound and electrical energy. The generator 800 can provide holds for use of all device user interface controls. The generator 800 can be used in any orientation. The generator 800 can create pressure waves within the fluid medium used to fill the balloon 100. The generator 800 can interface with fluids common to procedures. The generator 800 can interface with fluids such as balanced salt solution (BSS) and 50/50 contrast. The generator 800 can be an electric generator. The generator 800 can interface with a power supply. These and other advantageous features are described herein.

### Housing

The generator 800 can include a proximal handle. The generator 800 can include components that produce pressure waves for calcium fracturing. The generator 800 can include the membrane 884, the piston 878, and the cam hammer 848, among other components described herein. The proximal handle can be located proximal to the balloon 100, such that pressure waves generated by the generator originate proximal to the balloon 100. The proximal handle can be held by one hand of the user. The controls of the generator 800 can be accessed by the user when held by the user. The proximal handle can be coupled to the catheter 600 coupled to the balloon 100. The generator 800 can have components that move the membrane 884 to produce the pressure wave with ultrasonic frequency. In some embodiments, each of a frequency and an amplitude of the pressure wave is adjustable via the proximal handle.

The generator 800 can include a top housing 802. The top housing 802 can include a slot 804. The slot 804 can guide a speed control 810 as described herein. The slot 804 can include detents. The top housing 802 can include a flexible overmold to engage features of a printed circuit board, such as a button. The top housing 802 can be transparent to allow light transmission from a printed circuit board. The top housing 802 can include viewing ports to allow an LED light display. In some embodiments, the top housing 802 can include a display panel. The top housing 802 can include features to limit movement of internal components such as components of the generator. The top housing 802 can include features to limit rotation of internal components such as components of the generator. The top housing 802 can include a bearing surface for rotating components such as components of the generator.

The generator 800 can include a bottom housing 806. The top housing 802 and the bottom housing 806 can be secured. In some embodiments, the top housing 802 and the bottom housing can be secured with an ultrasonic weld. In some embodiments, the top housing 802 and the bottom housing 806 can be secured with a mechanical fastener such as a screw, snap, or rivet. In some embodiments, the top housing 802 and the bottom housing 806 can be secured with an adhesive. The bottom housing 806 can include features to limit movement of internal components such as components of the generator. The bottom housing 806 can include features to limit rotation of internal components such as components of the generator. The top housing 802 can include a bearing surface for rotating components such as components of the generator. The top housing 802 and the bottom housing 806 can prevent physical access to internal components. The top housing 802 and the bottom housing 806 can prevent physical access to connectors. The bottom housing 806 can include an orientation feature 808. The orientation feature 808 can be one or more fins projecting from the bottom housing 806. The orientation feature 808 can facilitate an upright orientation of the generator 800 during use. The orientation feature 808 can facilitate user access to controls during use. The orientation feature 808 can be configured to lie flat on a surface in some embodiments. The top housing 802 and the bottom housing 806 can maintain integrity of the generator 800 during use. The top housing 802 and the bottom housing 806 can form the majority of the proximal handle. The top housing 802 and the bottom housing 806 can provide an enclosure for other components as described herein. The top housing 802 and the bottom housing 806 can form the proximal handle.

### Actuator

The generator 800 can allow the frequency of pressure waves to be adjusted. The generator 800 can include the motor 830 as described herein. The generator 800 can include features to change the rotational speed of the motor 830. The motor 830 can rotate the cam hammer 848 of the generator. The speed of the motor 830 can correlate with the percussive movement of the generator which creates the pressure waves.

The generator 800 can include the speed control 810. The speed control 810 can be a slider. The speed control 810 can be an actuator. The speed control 810 can slide within the slot 804 of the top housing 802. The slot 804 can guide the speed control 810. The slot 804 can guide the speed control 810 to discrete locations. The slot 804 can guide the speed control 810 to detents. The detents can correspond to distinct frequencies of the motor 830. The speed control 810 can be secured to additional internal components described herein that effectuate the change in frequency of the motor 830 as the speed control 810 is moved. The speed control 810 can provide a surface that can be actuated by the user. The speed control 810 can provide a surface that limits the slipping of the user's finger during adjustment. The speed control 810 can include a rough surface to reduce slipping. The speed control 810 can provide physical feedback for speed positions. In the illustrated embodiment, the speed control 810 can provide physical feedback for three speed positions. The slot 804 can include detents for three positions. In some embodiments, the three positions can correspond to 1 Hz, 2 Hz, and 3 Hz. These frequencies can correspond to rotational speed of the motor shaft 831 of the motor 830. The speed control 810 can change the frequency of the motor shaft 831. The speed control 810 can change the number of cycles or events per second. The speed control 810 can change the frequency of pressure wave generation. In some embodiments, the generator 800 can include visual feedback related to the position of the speed control 810. The generator 800 can include markings that indicate the position of the speed control 810. The generator 800 can include markings that indicate the corresponding frequency. The generator 800 can include lights that indicate the position of the speed control 810. The speed control 810 can maintain integrity during use.

The generator 800 can include a position sensor system to determine the position of the speed control. The position sensor system can be a magnetic position sensor system. The generator 800 can include a magnet 812 coupled to the speed control 810. In some embodiments, the magnet 812 can be secured to the speed control 810 by a heat stake. In some embodiments, the magnet 812 can be secured to the speed control 810 by a fastener such as screws or adhesive. The magnet 812 can provide feedback on position of the speed control 810. The magnet 812 can provide consistent magnetic field for interaction with a detector of the position sensor system. The position sensor system can utilize other types of sensors including potentiometric sensors, optical encoders, inductive sensors, among other sensor types.

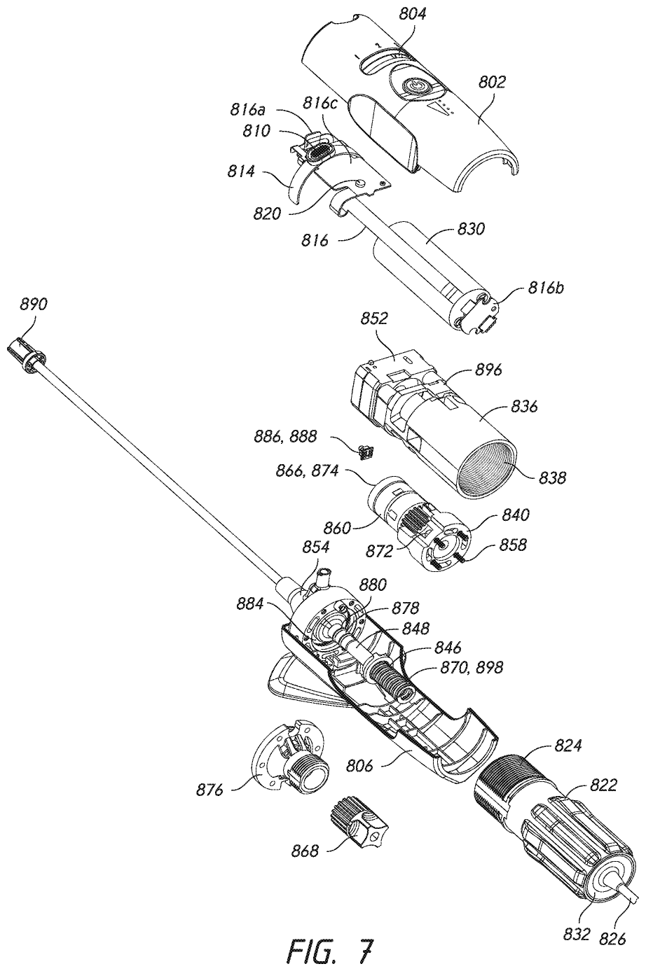

The generator 800 can include features to facilitate the movement of the speed control 810. The generator 800 can include a bearing strip 814 as shown in FIG. 7. The speed control 810 can be secured to the bearing strip 814. The speed control 810 can be secured within a slot of the bearing strip 814. The speed control 810 can form a snap fit with the bearing strip 814. The bearing strip 814 can rotate relative to the top housing 802 and the bottom housing 806. The bearing strip 814 can rotate within a slot within the top housing 802. The bearing strip 814 can rotate within a slot within other internal components. The bearing strip 814 can provide limited movement of the speed control 810. The bearing strip 814 can provide an acceptable user interface for the actuation force. The bearing strip 814 can maintain integrity during use. The top housing 802 can provide slots or guides for the bearing strip 814. The top housing 802 can provide a bearing surface for the bearing strip 814.

The speed control 810 and the bearing strip 814 can be coupled. The speed control 810 and the bearing strip 814 can move as a unit. The top housing 802 can include the slot 804. The speed control 810 can project through the slot 804. The bearing strip 814 can be positioned under the slot 804. The bearing strip 814 can rotate relative to the top housing 802. The bearing strip 814 can rotate within slots of the top housing 802 and internal components, such as a hammer bearing block 852 described herein. The bearing strip 814 can provide a surface to receive the actuation force of the user. The speed control 810 and the bearing strip 814 can slide relative to the top housing 802. The speed control 810 and the bearing strip 814 can slide to discrete positions. The top housing 802 can have detents which guide the speed control 810 to discrete positions. The speed control 810 and the bearing strip 814 can slide when the user interacts with the speed control 810. The speed control 810 and the magnet 812 can be coupled. The speed control 810 and the magnet 812 can move as a unit. The speed control 810 can control the speed of the motor 830. The user can control the speed of the motor 830 by sliding the speed control 810. The top housing 802 can include detents for a plurality of speeds of the motor 830. The user can visualize the speed of the motor 830 by viewing the location of the speed control 810. The user can visualize the speed of the motor 830 by viewing markings associated with the position of the speed control 810. In some embodiments, the generator 800 can have three speeds that can be selectable by the user. The three speeds can be 1 Hz, 2 Hz, or 3 Hz. Other configurations are contemplated.

### PCB

The generator 800 can include a printed circuit board assembly 816. The printed circuit board assembly 816 can be used to send electronic signals between components of the generator 800. The printed circuit board assembly 816 can include one or more printed circuit boards. The printed circuit board assembly 816 can include one or more processors. The printed circuit board assembly 816 can include one or more integrated circuits. The printed circuit board assembly 816 can route electrical signals from one point of the generator 800 to another point of the generator 800. The printed circuit board assembly 816 can include one or more subcomponents located within the generator 800 which control certain functions. The printed circuit board assembly 816 can include a main printed circuit board 816A. The printed circuit board assembly 816 can include a motor printed circuit board 816B. The motor printed circuit board 816B can control the motor 830. The printed circuit board assembly 816 can include a LED printed circuit board 816C. The LED printed circuit board 816C can control one or more LED lights. The LED printed circuit board 816C can include one or more LED lights which can be illuminated and viewed by the user. The top housing 802 can be transparent or have a transparent portion. The top housing 802 can allow light transmission from the LED printed circuit board 816C. The top housing 802 can include ports to allow transmission of LED light.

In some embodiments, the printed circuit board assembly 816 can be fixed relative to the top housing 802 and the bottom housing 806. In some embodiments, the main printed circuit board 816A can be fixed relative to the top housing 802 and the bottom housing 806. In some embodiments, the LED printed circuit board 816C can be fixed relative to the top housing 802 and the bottom housing 806. In some embodiments, the motor printed circuit board 816B can translate as the motor 830 translates. The printed circuit board assembly 816, or a portion thereof, can be fixed relative to internal components such as a cam bearing block 836 described herein. The printed circuit board assembly 816, or a portion thereof, can be fixed relative to internal components such as a hammer bearing block 852 described herein. The printed circuit board assembly 816, or a portion thereof, can be fixed relative to internal components such as a proximal printed circuit board cage 834.

The generator 800 can include the proximal printed circuit board (PCB) cage 834. The proximal printed circuit board cage 834 can be secured to the cable strain relief 832. In some embodiments, the proximal printed circuit board cage 834 can be secured to other internal components such as a cable strain relief 832 with one or more printed circuit board mounting screws 818. The proximal printed circuit board cage 834 can be secured to the motor printed circuit board 816B. In some embodiments, the proximal printed circuit board cage 834 can be secured to the motor printed circuit board 816B with a flexible snap. The proximal printed circuit board cage 834 can constrain the motor printed circuit board 816B away from the metal surface of the motor 830. The proximal printed circuit board cage 834 can be secured to other components with screws. The proximal printed circuit board cage 834 can be secured to the motor 830 with screws. The proximal printed circuit board cage 834 can maintain integrity during use. The printed circuit board assembly 816 can secure to the proximal printed circuit board cage 834 with snap features. The printed circuit board mounting screws 818 can secure the cable strain relief 832 to the proximal PCB cage 834. The proximal printed circuit board cage 834 can be secured to the motor 830 using snaps.

The printed circuit board assembly 816 can provide consistent positioning of one or more sensors that monitor rotation. The printed circuit board assembly 816 can provide consistent positioning of one or more sensors that monitor movement of components. The printed circuit board assembly 816 can provide consistent positioning of the detector of the position sensor system. The printed circuit board assembly 816 can include the detector of the position sensor system of the speed control 810. The printed circuit board assembly 816 can include the detector that can read the position of the magnet 812 of the speed control 810. The printed circuit board assembly 816 can be positioned within the generator 800 using manufacturing methods described herein. The printed circuit board assembly 816 can read a position of an internal component via a magnet. The printed circuit board assembly 816 can read the magnet 812 of the speed control 810. The printed circuit board assembly 816 can control the speed of the motor 830 based on the position of the magnet 812. The printed circuit board assembly 816 can change the speed of the motor 830 when the speed control 810 is moved. The printed circuit board assembly 816 can position the LEDs under the viewing ports of the top housing 802. The LEDs can illuminate to convey information to the user about the status of the device. The printed circuit board assembly 816 can be connected to input power. The printed circuit board assembly 816 can have the button 820 that can be depressed by the user through the top housing 802. The top housing 802 can have a flexible overmold to allow the user to engage the button 820. The top housing 802 can allow light transmission from the printed circuit board assembly 816. The printed circuit board assembly 816 can provide consistent positioning of a pressure sensor.

The printed circuit board assembly 816 of the generator 800 can be secured to internal components of the generator 800 using one or more printed circuit board mounting screws 818. The printed circuit board assembly 816 can be secured to internal components using snap features. The printed circuit board assembly 816 can be secured to internal components heat stake posts. The printed circuit board assembly 816 can retain the LED printed circuit board 816C with heat stake posts. The printed circuit board assembly 816 can be configured to accept DC power. In some embodiments, the printed circuit board assembly 816 can accept 22-30 VDC input power. The printed circuit board assembly 816 can provide a detachable connection to DC power. The printed circuit board assembly 816 can provide a detachable connection to one or more internal components. The printed circuit board assembly 816 can have a button 820 that interfaces with the top housing 802. The top housing 802 can include a flexible overmold to engage with the button 820. The button 820 can be actuated by the user. In some embodiments, the button 820 turns on and off a feature of the generator 800. The printed circuit board assembly 816 can enable software validation for firmware related functions. The printed circuit board mounting screws 818 can secure the printed circuit board assembly 816 to internal components such as stationary blocks. The printed circuit board mounting screws 818 can secure additional components such as components for strain relief. The printed circuit board mounting screws 818 can maintain integrity during use.

The generator 800 can include a controller that controls the operation of the components of the system 1100. The printed circuit board assembly 816 can include the controller. The controller can control the generator 800. The controller can comprise a microprocessor, a special or general purpose computer, a floating point gate array, and/or a programmable logic device. In some embodiments, the controller can be used to operate the generator 800 so that the range-of-pressure of the pressure wave delivered is at a suitable working pressure. The controller may also be used to improve the system 1100, for example, by limiting system pressures to be below thresholds and/or by limiting the time that the pressure remains in the high pressure state. In some embodiments, the controller may be used to vary or cycle the pressure of the fluid delivered to the balloon 100, for example, by cycling pressures provided by the generator. In some embodiments, sinusoidal or sawtooth pressure wave variability, as described herein, can be used to provide corresponding variability in the pressure delivered to the balloon 100. In some embodiments, the controller may regulate the frequence of the motor 830. The motor 830 can cause pulsations such as a percussion pulse or percussion wave. In some embodiments, the pressure wave comprises a percussive wave. In some embodiments, the pressure wave may include a series of discrete changes in the pressure pulses. In some embodiments, the pressure wave is a shockwave.

The controller advantageously can control the amplitude and frequency of the pulsations sent to the balloon 100 and to the serrated strips 200 with the raised features 300. The system 1100 may also include a user interface that outputs relevant system data and accepts user input. The user interface can be separate from the generator 800. The user interface can be integrated with the generator 800. For instance, the user interface can be disposed within the portable hand-held housing of the generator 800. In some embodiments, the user interface comprises a touch screen graphics display. In some embodiments, the user interface may display information related to the pressure wave. In some embodiments, the user interface may display information including the working fluid pressure. In some embodiments, the user interface may display information including instructions and/or procedures for operating on different disease types (e.g., calcified, fibrin, or a combination). The user interface may accept user input such as a time setting that sets a maximum time during which the system 1100 can deliver pressure wave. In some embodiments, an operator can select a ramp up and/or ramp down option in which the working fluid pressure can be gradually increased or decreased, respectively.

### Balloon

Due to the vastly different anatomical regions that use angioplasty, the design of balloons for angioplasty can include numerous advantageous specifications and specific balloon characteristics. One specification that can be of particular interest to the embodiments described herein is balloon compliance. Balloon compliance can be defined as the growth of the balloon in correlation to the pressure applied. Therefore, as the pressure increases, so does the balloon diameter and length. The incremental increase in growth can be controlled to some degree by the materials used, processes used in pre balloon blowing, and in the balloon blowing process. Independent of the controls put in place to control the balloon growth, there can be a direct correlation between the pressure applied to the balloon and the diameter and length of the balloon.

The balloon compliance can be referred to three distinct categories: non-compliant, semi-compliant, and compliant. Non-compliant (high-pressure) balloons can be commonly made of polyester or nylon or composite layers of material. Non-compliant balloons can be used for applications in which the balloon needs to expand to a specific diameter and exert high pressure to open a blockage or to dilate the vessel with little observable growth in the balloon size. Semi-compliant (mid-pressure) balloons can be commonly made of nylon or higher durometer polyurethanes. Semi-compliant balloons can be used in applications that require mid-high pressures but need more compliance than a non-compliant balloon and more flexibility to ease delivery and provide a range of balloon sizes related to the pressure delivered. Compliant (elastomeric) balloons can be made of polyurethane or silicone. Compliant balloons can be inflated by regulated volume instead of pressure. Compliant balloons may be inflated from 100%-800%. Compliant balloons can be used in applications that require the balloon to fully conform to or occlude the anatomy.

The effectiveness of balloon compliance in various disease morphology has been documented. For example, highly diseased and hardened arteries require higher pressure balloons to yield. Compliant and some semi-complaint balloons can reshape to the vessel wall generating the appearance of a “dog bone” with the diseased part of the vessel not yielding to the pressure being delivered. The use of non-compliant balloons allows for increases in pressure with minimal or no “dog-bone” ballooning behavior. By limiting the balloon to retain its shape and not conform to the vessel wall, the pressure can be delivered to the hardened, diseased region of the vessel. As the physician increases the pressure of the balloon, the balloon applies pressure to the region of the diseased vessel enabling the disease to yield under the load.

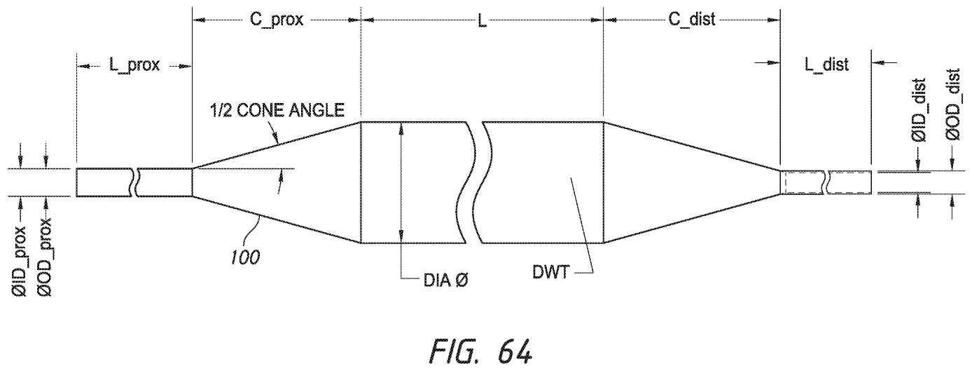

The balloon 100 can include various dimensions as shown in FIG. 64. The cylindrical balloon length (L) can range from 8 mm to 300 mm. The balloon diameter (DIA) can range from 1.0 mm to 30 mm. The Double Wall Thickness (DWT) can be minimized to obtain the smallest deflated state in balance with the maintaining functional performance of the balloon (e.g. burst resistance). The Double Wall Thickness (DWT) and material properties can be selected to limit stress (e.g. hoop stress) and manage growth rate of the balloon at various pressures. Materials such as polyvinyl chloride, cross-linked polyethylene, polyethylene terephthalate, nylon, polyurethane or other synthetic or co-polymers in combination of balloon DWT between 0.25 mm and 0.5 mm can be used. The balloon cone length (shown as C_prox and C_dist) can be a direct relation to the ½ cone angles range from 12-24 degree angles as shown giving a balloon cone length range of 1.0 mm to 10 mm. The balloon leg dimensions (e.g. ID_prox, OD_prox, and L_prox) can be as follow: ID_prox can range from 0.5 mm to 2.2 mm, OD_prox can range from 0.5 mm to 2.5 mm, and L_prox can range from 2.5 mm to 25 mm.

Specialty balloon designs included shaped structures on the outer surface of the balloon in part intended to address these limitations of plain balloons. The raised features 300 can include unhoned outward facing surfaces. The raised features 300 can be microperforation elements. The raised features 300 can be wedge shaped. The raised features 300 can be blunt. The raised features 300 can function as a hammer, not a chisel. In some embodiments, the raised features 300 can be blunt to pound against the tissue. In some embodiments, the raised features 300 can be blunt to avoid slicing or scoring the tissue. In some embodiments, the raised features 300 do not have a cutting edge. In some embodiments, the raised features 300 do not have a honed or sharpened edge. The raised features 300 can include an impaction surface. The raised features 300 can be used to strike the plaque. The raised features 300 can include a blunt face for striking. The raised features 300 can comprise metal. The strip 200 can include an outward facing surface. The strip 200 can include a blunt surface between the raised features. The strip 200 can function as a hammer. The strip 200 can include an impaction surface. The strip 200 can be used to strike the plaque. The strip 200 can include a blunt face for striking. The strip 200 can comprise metal. In some embodiments, the raised features 300 can function as an impact drill. In some embodiments, the raised features 300 can move at rate of speeds nearly equal to the speed of sound in the fluid the wave is traveling through. In some embodiments, the raised features 300 can move at the speed of sound in a particular direction. In some embodiments, the raised features 300 can move at higher sound velocities due, in part, to the stiffness of the material. In some embodiments, the raised features 300 can move at ultrasonic frequency.

In some embodiments, the balloon 100 can be a single layer balloon. In some embodiments, the balloon 100 comprises a base balloon 102 and an outer retention balloon 104. The outer retention balloon 104 can facilitate the retention of the strips 200. The raised features 300 of the strips 200 can rise above the outer surface of the outer retention balloon 104. The raised features 300 can be in direct contact with the plaque and the vessel wall when the balloon 100 is inflated. In some embodiments, both the base ballon 102 and the outer retention balloon 104 have the same compliance, allowing similar expansion under the same pressure load. In some embodiments, the base ballon 102 and the outer retention balloon 104 have a slightly different compliance, where the two balloons have dissimilar expansion ratios under the same pressure load.

Through the use of balloon 100, the changes in pressure produced by the pressure waves can be effectively applied to repeatably drive the raised features 300 of the serrated strips 200 into the diseased tissue. The balloon 100 can drive the raised features 300 of the serrated strips 200 directly connected to the balloon 100. The balloon 100 can drive the raised features 300 of the serrated strips 200. The raised features 300 of the serrated strips 200 can impact with each pressure wave. The raised features 300 of the serrated strips 200 can produce a tapping motion under the repeated influence of multiple pressure waves. The use of an alternating driving force by the pressure waves can cause tapping of the diseased tissue. The tapping can drive energy through the diseased cellular matrix of the plaque which can force fracturing of the plaque. Tapping the diseased tissue and driving energy through the diseased cellular matrix of the plaque can cause the eventual yielding of the plaque under the repeated loads. The tapping motion reduces the time required to yield the hardened diseased tissue. The tapping motion can accelerate the creation of microperforations in the plaque. The tapping motion can accelerate the creation of cracks along cleavage planes. The tapping motion can create fracturing of the plaque through the propagation of waves. The tapping motion can reduce the time needed to create cracks between the microperforations. The tapping motion can reduce the time needed to break calcium bonds. The reduced time can be particularly useful with certain vessels, such as those that supply blood to the heart.

The balloon 100 can interact with the wall of the vessel. The body of the balloon 100 can be in direct contact with the vessel when the balloon 100 is inflated. The balloon 100 can undulate with the motion of the pressure wave. The balloon 100 can undulate at the speed of sound. The undulation can be rapid. The undulation can be a wavelike movement. The balloon 100 can resonate. The balloon 100 can reverberate. The balloon 100 can vibrate, wherein the vibrations can be caused by sound or echo. The balloon 100 can expand and contract with the pressure wave. The balloon 100 can vibrate against the surface of the vessel. Some designs can intentionally generate a surface motion of the balloon 100 while the balloon 100 remains engaged against the vessel wall. In some embodiments, the balloon 100 produces vibrational motion on the surface of the wall. The movement of the balloon can impart a force on the vessel which is separate and distinct from the force of the raised features 300 of the strips 200. The movement of the ballon 100 can be induced by the pressure wave that travels through the body of the balloon 100. In some embodiments, the pressure wave moving through the liquid filled system can induce a translation of energy to the strip 200 and the raised features 300 including but not limited to vibrating the raised features 300 of the strips 200. The movement of the raised features 300 of the strips 200 can be induced by the pressure wave that travels through the body of the balloon 100.

The pressure wave within the body of the ballon can have a longitudinal component. Within the fluid used to fill the balloon 100, the pressure wave can be exclusively or predominantly longitudinal. The transverse component of the pressure wave does not propagate within the fluid of the balloon 100. The body of the balloon 100 can be a solid surface. In the body of the balloon 100, the pressure wave can propagate with both a longitudinal and transverse component. The pressure wave can have a complex wave motion. Upon interaction with the pressure wave, the balloon 100 can allow particles of the balloon material to vibrate in the longitudinal direction of the wave propagation and perpendicular to the direction of wave propagation. This bi-direction movement can cause the vibrational movement of the balloon 100 against the wall of the vessel. The strips 200 and the raised features 300 can be a solid surface. In the strips 200 with raised features 300, the pressure wave can propagate with both a longitudinal and transverse component. The pressure wave can have a complex wave motion. Upon interaction with the pressure wave, the strips 200 and the raised features 300 can allow particles of the material of the strips 200 and the raised features 300 to vibrate in the longitudinal direction of the wave propagation and perpendicular to the direction of wave propagation. This bi-direction movement can cause the vibrational movement of the strips 200 and the raised features 300 against the wall of the vessel.

The pressure wave can change the shape of the balloon 100. The changes in the balloon pressure from the pressure wave, independent of the amount, frequency, or waveform appearance that the pressure takes, can all induce a change in the balloon shape. The shape change can be in the form of a diameter shift, a length shift, or a combination of relative motion of both diameter and length of the body of the balloon 100. In the design and manufacturing of medical balloons, the polymer strands can be oriented to allow for the balloons to stretch unidirectionally. Some designs enable the balloon to expand more typically radially. Some designs enable the balloon to expand more typically longitudinally. Some designs enable the balloon to expand more typically bidirectionally. Some designs enable the balloon to expand more typically longitudinally and radially.

As the pressure is applied to the inner balloon surface from the pressure wave, the material within the balloon matrix can stretch to release the growing pressure. The balloon matrix material stretch can occur at the molecular bond level. The multiple layers of molecules that form the balloon 100 begin to elongate, stretching the long molecular bonds of polymers that make up the material of the balloon 100. This internal molecular elongation event enables the diameter and/or length of the balloon 100 to grow without rupturing the balloon 100. The balloon 100 can change based on the parameters of the pressure wave generated by the systems described herein. The balloon 100 can change based on the pulsatile pressure generated by the generator 800. The balloon 100 can change based on the direction that the pressure waves propagate through the generator 800 to the balloon 100. The pressure wave can propagate along the length of the balloon 100 without a reduction amplitude of the pressure wave between the proximal and distal end of the balloon 100. In some embodiments, the pressure wave can propagate along the length of the balloon 100 with the same parameters at the proximal end of the balloon body, at the middle of the balloon body, and at the distal end of the balloon body. In some embodiments, the pressure wave can propagate along the length of the balloon 100 with accelerated or decelerated speed at the cones of the balloon induced by the shape of the cones.

The nature of the material choices and the method for manufacturing the balloon 100 can provide the structure which enables the correlation between the pressure delivered and the growth of the balloon 100. Therefore, designs can be made to effect the body of the balloon 100 directly in reaction to the pressure wave in some embodiments. In some embodiments, designs can pulsate the diameter of the balloon 100 as the pressure changes with the pressure waves. In some embodiments, designs can pulsate the length of the balloon 100 as the pressure changes with the pressure waves. In some embodiments, designs can have minimal impact on the diameter of the balloon 100 as the pressure changes with the pressure waves but the balloon design more effectively translates the energy to the strip 200. In some embodiments, designs can have minimal impact on the diameter of the balloon 100 as the pressure changes with the pressure waves but the balloon design more effectively translates the energy to the raised features 300 of the strip 200. In some embodiments, designs can have both an impact on the diameter of the balloon as the pressure changes with the pressure waves as well as the balloon design effectively translates the energy to the body of the strip 200 and the raised features 300. The delivery of energy to the balloon 100, the strip 200, and/or the raised features 300 of the strip 200 produces an effect on the ability of the serration balloon to more effectively crush or fracture diseased vessels. The pressure waves delivered to the strips 200 engages the strips 200 into the vessel more effectively, produces a pathway for the strips 200 to tap or hammer the strips 200 into the diseased state of the vessel. The pressure waves delivered to the balloon 100 offers an effective stretching mechanism for the balloon 100 that when engaged with the vessel surface provides a series of forces including radial, longitudinal, and tangential forces. As these forces are applied and then removed in a periodic fashion, the diseased state of the vessel can weaken. The disease state can be a tight interconnected lattice of hydroxy-apatite, a weave of fibrin, an unstructured body of plaque, or a combination of one or more of these diseased states. In the case of thick, hardened, or heavily burdened atherosclerosis disease, the ability to crack or crush the diseased vessel can be provided. In the case of thrombin disease, the ability to tear the fibrin can be provided. While, in the more complex atherosclerotic disease, the design of the system 1100 can offer more than one modality to open the vessel and increase blood flow. In complex atherosclerotic disease, the pulse enhanced serration angioplasty can modifies the vessel, by utilizing the pressure wave to push the complex atherosclerotic disease out of the vessel flow region. By enabling the vessel to expand under low pressure, the pulse enhanced serration angioplasty systems described herein can enable a space for the complex atherosclerotic disease to occupy within the vessel matrix providing effective lumen growth lumen expansion.

The balloon 100 can have features to enable delivery to the treatment site. In some embodiments, balloon 100 can be pleated and crimped down to the very narrow profile allowing the ballon 100 to be delivered through and introducer sheath with a narrow diameter. The balloon 100 can have features to enable retrieval from the treatment site. Once the balloon 100 has been deployed and deflated, the post-inflated balloon profile can be larger than its original pleated and crimped down diameter. This new profile may have strips 200 that sit proud of the balloon profile potentially scraping the arterial wall or snagging on the opening of an accessory device such as an introducer sheath. In some embodiments, the strips 200 facilitate folding of the strips under flaps of the balloon for retraction.

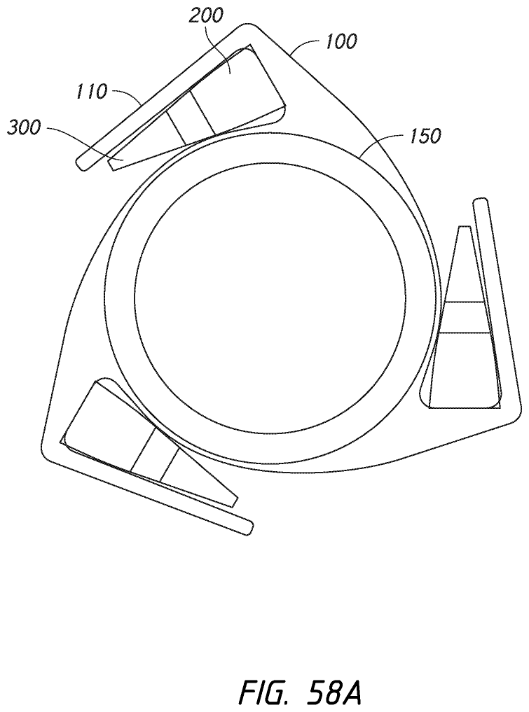

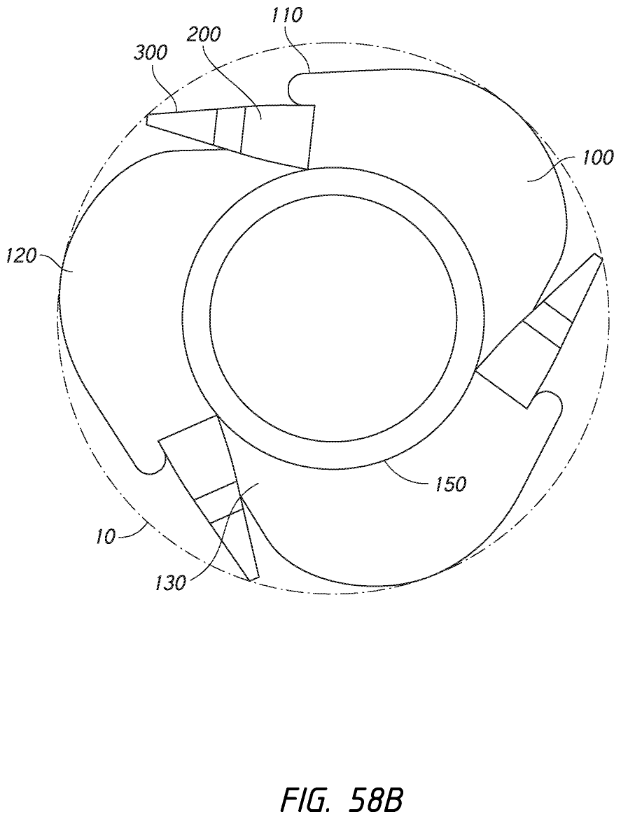

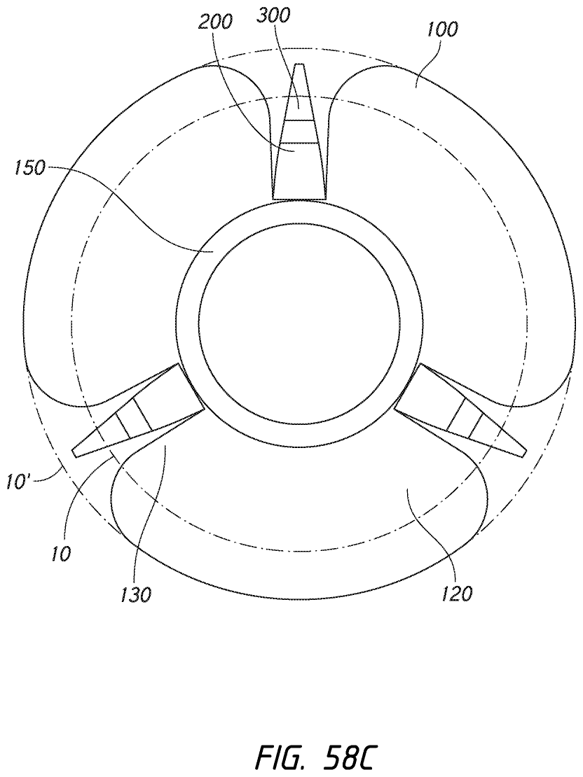

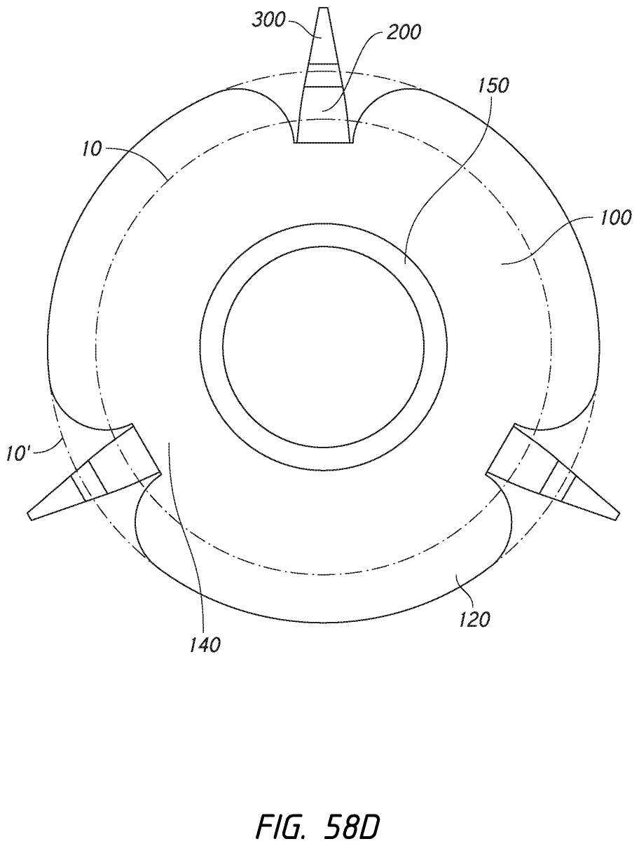

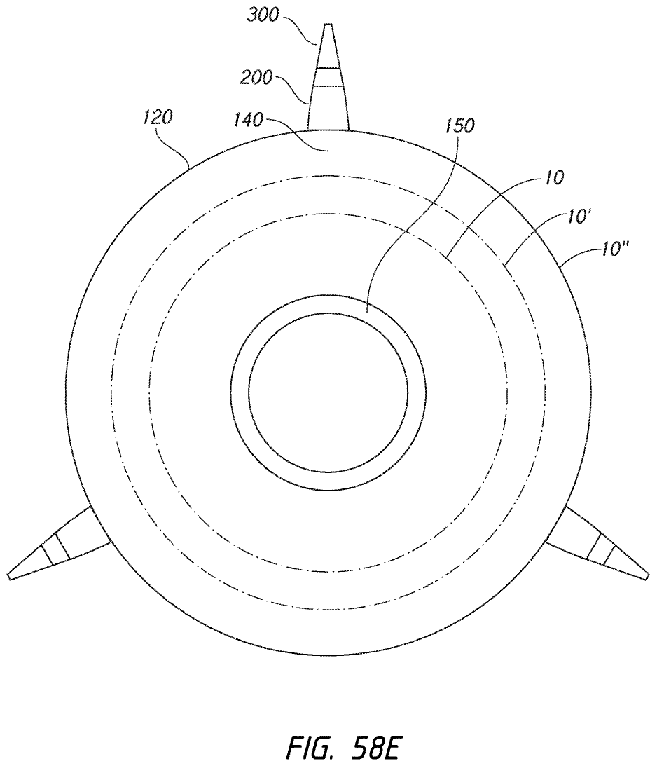

The balloon 100 can have features to orient the strips 200 and the raised features. The balloon 100 can be inflated according to FIG. 58A-58E described herein, which show the mechanism for serrated strip elements 200 to turn from a tangential orientation under the wing of the balloon material to being uncovered and orienting perpendicularly within a very tight stenotic lesion. This series of illustrations shows the delivery configuration of the balloon and subsequent expansion. As an overview, the strips 200 rotate during expansion of the balloon 100. The strips 200 can be delivered in a tangential orientation. The strips 200 can be at least partially or fully covered by the balloon 100 during delivery. The strips 200 lie down during delivery. The expansion of the balloon can cause the strips 200 to turn from the tangential orientation to the perpendicular orientation. Additionally, the deflation of the balloon can cause the strips 200 to turn from the perpendicular orientation to the tangential orientation. The strips 200 can rotate within vessels with very small diameters. The strips 200 can rotate within small diameter stenotic lesions. The mechanics of the expansion of the balloon 100 facilitate the serration and subsequent crack propagation. These figures illustrate how the balloon expansion occurs.

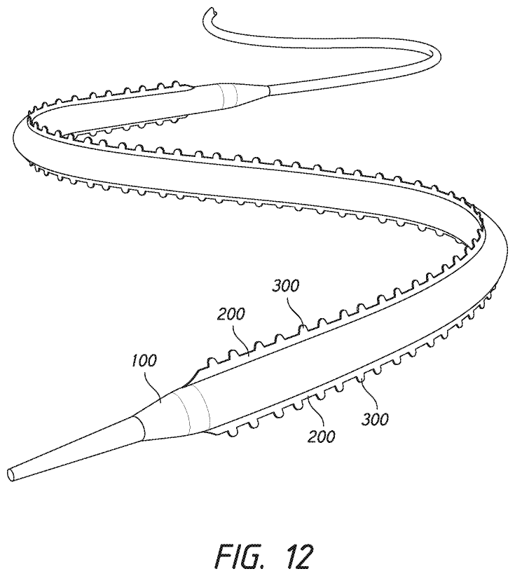

The balloon 100 can include any number of strips 200 with raised features. FIG. 12 illustrates an embodiment of a balloon 100. The balloon catheter 100 can include two strips 200. The balloon 100 consists of only two strips 200. The two strips 200 can be equally spaced around the circumference of the balloon. The two strips 200 can be separated by approximately 180 degrees. The strips 200 can have any shape described herein. The strips 200 can include a plurality of raised features 300. Each raised feature can include radially outward facing surface. Each raised feature can include an unhoned surface. The raised feature can have any shape described herein. The balloon 100 can be coupled to raised features 300. The strips 200 with the raised features 300 can be disposed on the balloon 100. The strips 200 with the raised features 300 can be directly connected to the balloon 100. The balloon catheter 100 can include one strip 200. The balloon 100 consists of only one strip 200. The balloon catheter 100 can include zero strips 200. The strips 200 can be disposed on a separate carrier 1200 as described herein. The raised features 300 can be disposed on a separate carrier 1200 as described herein.

The raised features 300 can be configured to provide a series of oriented punctures or serrations into a diseased vessel wall. The reduction to two strips 200 can provide two lines of oriented punctures or serrations. The two lines can create predictable and controlled lumen expansion along the two serrated lines. In some embodiments, the balloon 100 consisting of only two strips 200 can be as effective as balloons 100 with more strips 200. In some embodiments, the balloon 100 consisting of only two strips 200 can have the same translation of energy as balloons 100 with more strips 200. The raised features 300 of each strip 200 can produce a linear line of weakness or perforations without cutting a continuous axial segment of the vessel wall. The strips 200 can enable an effective vessel lumen expansion of vessels below the knee. The balloon 100 can be inflated and the balloon 100 can unfold in the vessel below the knee. The strips 200 can rotate from a tangential orientation to a perpendicular orientation within the vessel. The lobes 120 of the balloon 100 can continue to expand and press against the strips 200 and the vessel wall. The raised features 300 can contact the vessel wall. The narrow profiles of the raised features 300 can penetrate the vessel wall generating nucleation sites for the fissuring event. The mechanics of crack propagation can accelerates the fissuring event for vessels below the knee. The mechanics of tensile force of the lobes 120 can accelerates the fissuring event for vessels below the knee. The fissures can produce cracking along the intra-luminal surface along the two serrated lines. The cracks can join longitudinally along the serrated lines due to the proximity and alignment of the cracks. The cracks can join to become a long crack along the intra-luminal surface corresponding to each strip 200. The cracks can extend along the entire length of the strip 200, or less of the strip length, or greater than the strip length. The depth of the penetration of the crack can be typically similar to the depth of medial tissue. There can be two longitudinal cracks produced in the tissue for vessels below the knee.

The balloon 100 can have a diameter and length based on the vasculature to be treated. The balloon 100 can be configured to be delivered to any vessel with plaque buildup. The balloon 100 can be configured to be delivered to vasculature of the heart. The balloon 100 can be configured to be delivered to the coronary arteries. The balloon 100 can be configured to be delivered to the left main coronary artery. The balloon 100 can be configured to be delivered to the left anterior descending artery. The balloon 100 can be configured to be delivered to the left circumflex artery. The balloon 100 can be configured to be delivered to the right coronary artery. The balloon 100 can be configured to be delivered to carotid arteries. The balloon 100 can be configured to be delivered to the abdominal aorta. The balloon 100 can be configured to be delivered to the renal arteries. The balloon 100 can be configured to be delivered to the mesenteric arteries. The balloon 100 can be configured to be delivered to the vertebral arteries. The balloon 100 can be configured to be delivered to peripheral arteries. The balloon 100 can be configured to be delivered to vasculature above the knee. The balloon 100 can be configured to be delivered to the common femoral artery. The balloon 100 can be configured to be delivered to the superficial femoral artery. The balloon 100 can be configured to be delivered to the deep femoral artery. The balloon 100 can be configured to be delivered to the popliteal artery.

The balloon 100 can be configured to be delivered to vasculature below the knee. The balloon 100 can be configured to be delivered to blood vessels located in the lower leg. The balloon 100 can be configured to be delivered to the arteries and veins that are below the knee joint. The balloon 100 can be configured to be delivered to vasculature in the foot. The balloon 100 can be configured to be delivered to the popliteal artery. The balloon 100 can be configured to be delivered to the anterior tibial artery. The balloon 100 can be configured to be delivered to the peroneal artery. The balloon 100 can be configured to be delivered to the posterior tibial artery. The balloon 100 can be configured to be delivered to the infrapopliteal vessels. The balloon 100 can be configured to be delivered to tibiopedal vessels located below the knee. The balloon 100 can be configured to be delivered to a vessel that supplies blood to the lower leg. The balloon 100 can be configured to be delivered to a vessel that supplies blood to the foot. The balloon 100 can be configured to treat peripheral artery disease.

Treatments can be particularly challenging below the knee. The vessels below the knee produce procedural challenges based on the size of the vessels. The vessels below the knee produce procedural challenges based on the tortuous bends of the vessels. The patients that require treatment may have additional challenges, such as critical limb ischemia. The balloon 100 can be sized to be delivered to the treatment site. The procedure can include contralateral common femoral artery access. The procedure can include antegrade ipsilateral femoral artery access. The balloon 100 can be passed through the vasculature to the treatment site. The balloon 100 can be positioned within the target vessel. The balloon 100 can be expanded and the raised features 300 can penetrate the plaque, similar to methods described herein. The balloon 100 for below the knee vasculature can be smaller than other balloons for above the knee vasculature. The balloon can have a 20 mm balloon length. The balloon can have a balloon diameter of 0.5 mm, 1 mm, 1.5 mm, 2.0 mm, 2.5 mm, 3 mm, 3.5 mm, or any range of two of the foregoing values.

In some embodiments, the balloon 100 with only two strips 200 can offer advantages to aid in delivery of the balloon 100. The two strips 200 can be diametrically opposed. The balloon 100 can be self-orienting within the vessel. The balloon 100 can be self-orienting to have low strain on the strips 200. The balloon 100 can be self-orienting to have less bending of the strips 200. The balloon 100 can rotate within the vessel to orient the two strips 200. The strips 200 self-orient away from being aligned with the curve of the vessel. The strips 200 self-orient to be tangential to the curve of the vessel. While not to be bound by a theory, the balloon 100 can self-orient in response to the bending force apply to the strip 200 as the strip 200 navigates tortuous anatomy. The balloon 100 can be free to rotate in response to the bending force. The balloon 100 can rotate toward a low stress orientation relative to the curved anatomy. The low stress orientation can have the strips 200 tangential to the curve of the vessel. The strips 200 experience lower stress when tangential to the curve of the vessel than aligned with the curve of the vessel. The strips 200 can continue to reorient themselves along various curves of the vessel on the way to the treatment site. At each curve of the vessel, the strips 200 can self-orient toward a lower stress state in which the strips are not aligned with the curve, but rather offset relative to the curve. The strips 200 orient themselves to the least energy. The strips 200 orient themselves to be lateral to the curve of the vessel. The strip 200 can be oriented laterally relative to the curve of the vessel.

Not to be limited by theory, but the balloon 100 consisting of only two strips 200 has the advantage of self-righting within curved vessels. In some embodiments, balloon catheters with three or more strips may not be able to self-orient since at least one strip would be aligned with the curve of the vessel. In some embodiments, balloon catheters with three or more strips may not be able to rotate toward a lower stress orientation within the vessel. The balloon 100 consisting of only two strips 200 can rotate such that the strips 200 are oriented ninety degrees relative to the curve of the vessel. Not to be limited by theory, but the balloon 100 consisting of only two strips 200 may be lighter and easier to rotate due to the reduced number of strips. Balloon catheters with three or more strips have additional rigidity due to the number of strips. The balloon 100 consisting of only two strips 200 may be less rigid and easier to self-orient within the vessel.

### Raised Features

The systems described herein can have raised features 300. In some embodiments, the raised features 300 can be longitudinally oriented which may have advantages when interacting with the pressure wave. In some embodiments, the raised features 300 can have a significantly smaller surface area than the balloon 100 and the corresponding strip 200 which may have advantages when interacting with the pressure wave. In some embodiments, the raised features 300 can have a particular shape that may amplify pressure delivered by the pressure wave.

The shape of the raised features 300 can direct the longitudinal component of the pressure wave. The shape of the raised features 300 can direct the transverse component of the pressure wave. In some embodiments, the side walls of the raised features 300 along the width of the strip 200 can direct a component of the pressure wave. The side walls of the raised features 300 can act as mechanism for the transverse component of the pressure waves to be transmitted along the side walls. In some embodiments, the longitudinal walls of the raised features 300 along the length of the strip 200 can direct a component of the pressure wave. The longitudinal walls of the raised features 300 can enable the translation of the longitudinal component of the pressure waves. The shape of the raised features can direct components of the pressure waves along perpendicular directions. The shape of the raised features 300 can be configured to offer an advantage for the delivery of the pressure wave. The pressure wave can have the transverse component and the longitudinal component when the pressure wave travels in solid materials, such as the raised features 300. The raised features 300 can support both the longitudinal and transverse movement, allowing both components of the pressure wave to propagate.

The raised features 300 can be disposed on the strips 200 in some embodiments. The strips 200 can extend longitudinally along the balloon. The strips 200 can extend along the length of the balloon 100. In some embodiments, a series of strips 200 is disposed on the balloon 100. In some embodiments, the strips 200 may be a series of strips bonded in each row. For instance, if a single strip can be 120 mm long on the balloon 100, then the designs can have a series of four 30 mm strips or a series of three 40 mm strips or a series of six 20 mm strips. The series of strips 200 can be arranged longitudinally along the outer surface of the balloon 100. The series of strips 200 can be separated by a gap. Any combination of strip length plus separation between the strips, times the number of strips in a row would be a replacement of a single strip in any of the orientations around the balloon 100 as described herein. In some embodiments where the strips are fractions of the overall length separated by a gap between each strip fraction, the expansion and contraction of the balloon 100 by the pressure wave in the wave enhanced balloon angioplasty system can generate two innovative features. As the balloon 100 expands due to the pressure wave, the balloon 100 can grow both longitudinally and radially. In some embodiments, the radial growth can allow the balloon 100 to apply a force on the vessel wall, as described herein. The radial growth can have many advantages, such as accelerating crack propagation through tensile force on the vessel wall and the hammering motion of the balloon body. The effects of the longitudinal growth are typically minimized in designs where the strip length can be nearly equal to the balloon length. The strip 200 can limit or prevent growth longitudinally with the pressure wave when the strip length is similar to the length of the body of the balloon 100. In the strips arranged in series configuration, wherein a plurality of strips are arranged in a row, the growth of the balloon 100 increases the size of each gap between the strip fractions under the influence of the pressure wave. The balloon growth under the influence of the pressure wave allows each strip to move in both the radial direction and in the longitudinal direction. The strips arranged in series configuration can produce a longitudinal fractional plowing, scraping, or cutting like event when the balloon 100 grows longitudinally with the pressure wave. The strips arranged in series configuration can produce longitudinal scraping that behaves like an atherectomy catheter. The ability to crush the hardened atherosclerosis in the vessel in two directions, both radially and longitudinally, can have advantages. The advantages further advance the effectiveness of these embodiments to more quickly restore vessel inner diameter, volumetric flow through the vessel, and restore vessel compliance. In some embodiments, slightly offset strips or non-linear strips arranged in series are envisioned. In some embodiments, non-longitudinally oriented strips or strips with a cork screw like configured on the balloon surface can offer advantages in some diseased morphologies.

The raised features 300 and associated strip 200 can have features that facilitate serration with pressure waves. The raised features 300 and associated strip 200 can have features to function as rigid beams for pulsatile pumping. The raised features 300 and associated strip 200 can induce a more effective calcium fracturing along longitudinally-oriented line of weakness.

In some embodiments, the raised features 300 comprise a wedge dissector. In some embodiments, the raised features 300 comprise a wedge shape. In some embodiments, the raised features 300 can have any feature described herein. In some embodiments, the raised features 300 can be a scoring element. In some embodiments, the raised features 300 can be blunt. In some embodiments, the raised features 300 can contact a physical object, such as plaque.

In some embodiments, as illustrated for example in FIG. 13 which is a close-up detail view of an embodiment of a raised features 300 on its associated strip 200, a raised features 300 can include a strip-facing base surface 302 (which may also be referred to herein as a bounded surface). The strip-facing base surface 302 of the raised features 300 can be defined by the base where the raised features 300 protrude outward and directly continuous with a surface of the strip 200 at the interface between the raised features 300 and the strip 200. The strip could be a spline 200 or other strip-like structure. In some embodiments, this strip-facing base surface 302 has a relatively narrow width.

The raised features 300 and associated strip 200 can be made of a solid material. The raised features 300 and associated strip 200 can be made of a hard material. In some embodiments, the material can be martensitic stainless steel, with a hardness as low as 48 and as high as 70 but more typically between 52 to 64 on the Rockwell C-scale (HRC) although other materials including a polymer or co-polymer including but not limited to polyolefin, fluoropolymer (including fluorinated ethylene-propylene (FEP), polytetrafluoroethylene (PTFE) or polyvinylidene fluoride (PVDF) (e.g. KYNAR)), polyvinyl chloride (PVC), neoprene, silicone, elastomer or synthetic rubber and fluoropolymer elastomer (e.g. VITON), or a combination thereof can be utilized. The strip 200 and the raised features 300 can have hardness on the Rockwell C-scale (HRC) of 48, 49, 50, 51, 52, 53, 54, 55, 56, 57, 58, 59, 60, 61, 62, 63, 64, 65, 66, 67, 68, 69, 70, or any range of the foregoing values. In some embodiments, the strip can be about or no more than about 0.004″, 0.005″, or 0.006″ wide (oriented circumferentially). In some cases, the width can be between about 0.006″ and about 0.020″ or between about 0.004″ and about 0.030″. In some embodiments, the strip 200 typically runs longitudinally the length of the balloon 100. In some embodiments, the strip 200 can be oriented in angles up to and including 90 degrees from the longitudinal axis of the balloon 100. In some embodiments, the strip 200 can be oriented in a helical fashion at varying pitches. In some embodiments, the height of the base strip 200 can be between about 0.004″ and about 0.010″, or between about 0.002″. and about 0.020″ in some embodiments. The height of the base strip 200 can be equal to the height of the raised features 300 in some embodiments. The height of the base strip 200 can be less than the height of the raised features 300 in some embodiments. The height of the base strip 200 can be greater than the height of the raised features 300 in some embodiments.

Still referring to FIG. 13, a raised feature 300 can also include a radially outwardly facing surface 304 (which may be referred to herein as an unbounded surface or tip) that can define a top surface of the raised feature 300 from first (e.g., proximal) edge 306 to second (e.g., distal) edge 308 and be configured to contact tissue, plaques, or other structures within the body. Also shown are anterior surface 310, posterior surface 312, and opposing lateral surfaces 314 and 316. In some embodiments, the lateral surfaces 314, 316 extend upward generally perpendicular to the longitudinal axes of the strips, and the radially outward facing surface extends between the lateral surfaces as a linear, curved, or other geometry as described elsewhere herein at an angle to the lateral surface/lateral surface axis. The strips 200 can have an unbounded (e.g., superior-facing) surface 202 that can be coextensive with the strip-facing surface or boundary 302 of the raised feature 300. The strips 200 can have side surfaces 204. The strips 200 can have an inferior-facing surface 203. In some embodiments, the radially outwardly facing surface 304 can facilitate delivery of pulsatile energy of the pressure wave through the surface area of the raised features 300 having an unhoned width along a length. In some embodiments, the anterior surface 310, posterior surface 312, and opposing lateral surfaces 314 and 316 can facilitate delivery of pulsatile energy of the pressure wave through the wedge shape. In some embodiments, the raised feature 300 can facilitate delivery of pulsatile energy of the pressure wave through the rigid material of the raised feature 300. In some embodiments, the raised feature 300 can facilitate delivery of pulsatile energy of the pressure wave through the shape of the raised feature 300.

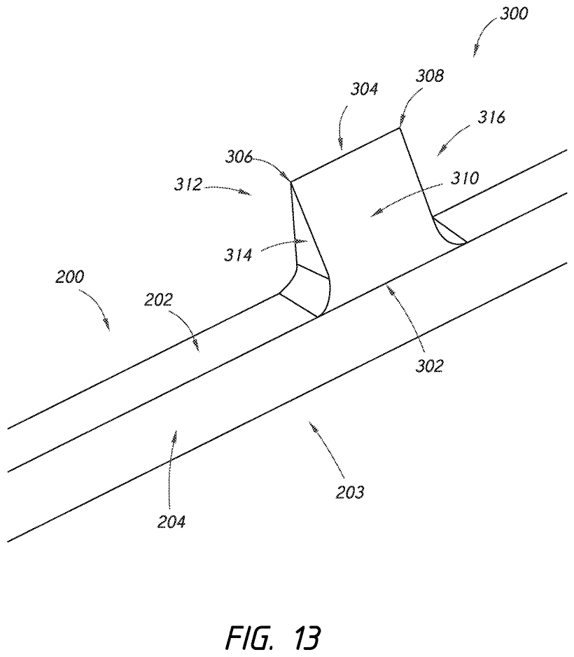

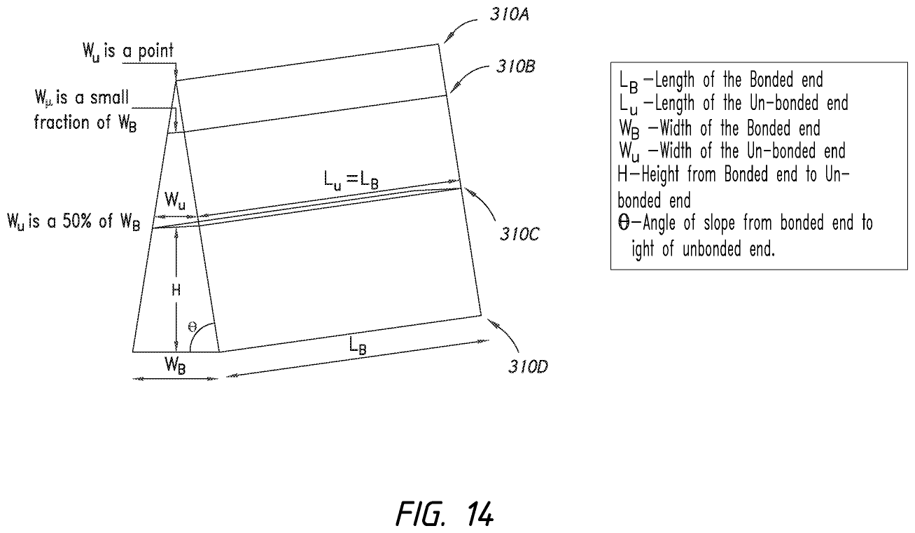

FIG. 14 is a schematic illustrating several possible non-limiting embodiments of the raised feature 300. In some embodiments, the length of the radially outwardly facing surface L_{U} (e.g., radially outwardly facing surface 304 between first edge 306 and second edge 308 of FIG. 13) can be between about 30%, 20%, or 10% less than the total length of the strip-facing surface L_{B} (of strip-facing surface 302 in FIG. 13). In some embodiments, the radially outwardly facing surface length L_{U} can be from about 50% to about 20% less than the strip-facing surface length L_{B}, and sometimes as large as the strip-facing surface length L_{B}. The radially outwardly facing surface width W_{U} can be in some cases equal to or less than the strip-facing surface width W_{B}, and typically between or less than about 10%, 20%, 30%, 40%, or 50% of the strip-facing surface width W_{B}, or between about 20% and about to 50% less than the strip-facing surface width W_{B}, and sometimes about or up to about 50%, 60%, 70%, 75%, or 80% of the strip-facing surface width W_{B}. The radially outwardly facing surface width W_{U} can be in some cases equal to or less than 0.01 inch. The radially outwardly facing surface width W_{U} can be in some cases equal to or between 0.005 inch and 0.002 inch, or 0.002 inch and 0.001 inch, or 0.0012 inch and 0.0002 inch. The radially outwardly facing surface width W_{U} can be in some cases equal to 0.0011 inch, 0.0009 inch, 0.0008 inch, 0.0007 inch, 0.0006 inch, 0.0005 inch, 0.0004 inch, or 0.0003 inch. In some embodiments, the radially outwardly facing surface area (L_{U}×W_{U}) can be from about 30% to about 10% less than the strip-facing surface area (L_{B}×W_{B}). Therefore, in some embodiments there can be an angle θ that is equal to or less than about 90 degrees that defines the slope from the strip-facing surface width W_{B} to the radially outwardly facing surface width W_{U} on at least one of the strip-facing surface width W_{B} edges. While in some embodiments the radially outwardly facing surface width W_{U} can be constant from edge to edge, in some embodiments the radially outwardly facing surface width W_{U} varies along the radially outwardly facing surface length L_{U} as described elsewhere herein, such as decreasing from a first lateral edge to a point or segment in between the first lateral edge and the second lateral edge of the radially outwardly facing surface segment, and then increasing, from the point or segment in between the proximal edge and the distal edge, to the distal edge. In some embodiments, the relatively central segment in between the proximal edge and the distal edge has a constant width, while the lateral segments surrounding relatively central segment have variable, such as tapered widths. The raised features 300 can be micro wedges.

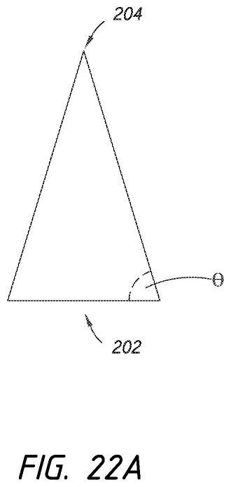

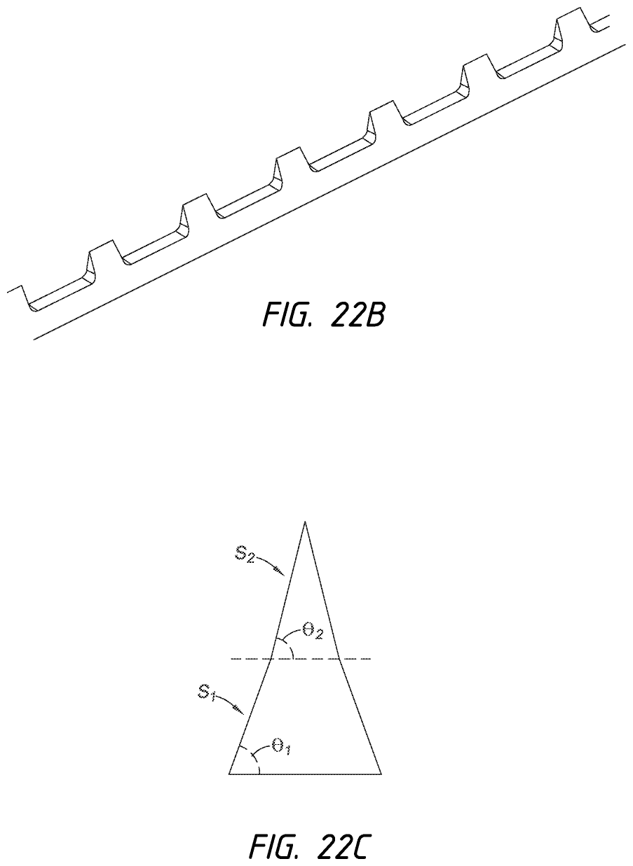

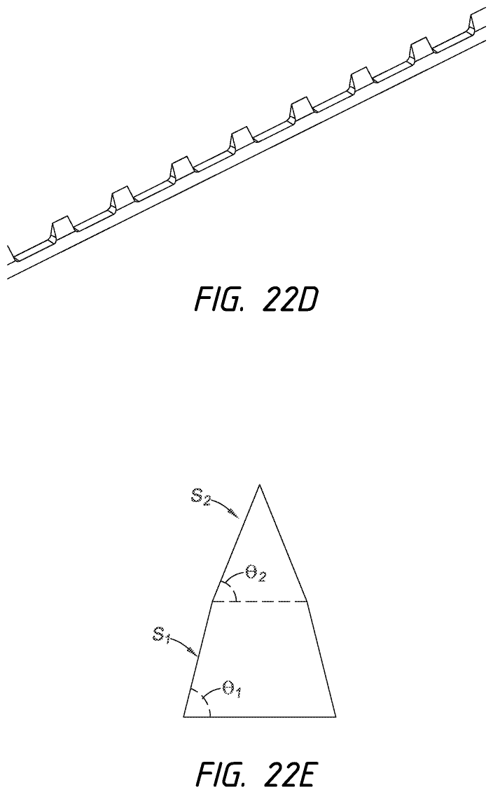

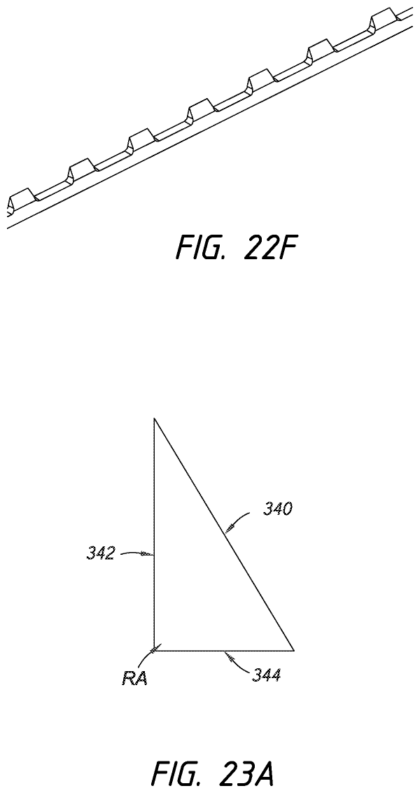

Although the radially outward facing width W_{U} can come to a point, sloping from the strip-facing base width W_{B} of the strip-facing base surface 302 to the radially outward facing width W_{U} of the radially outward facing surface 304 in a single, constant sloped angle θ or bevel such as shown in FIG. 22A (end view resembling an isosceles triangle) and FIG. 22B (isometric view), the raised feature 300 can also in some embodiments include a plurality of different angles, such as more than a single slope angle such as a double, triple or more bevel (e.g., a first angle for a first segment of the height, a second angle for a second part of the height that can be less than or greater than the first angle, and in some cases a third angle for a third part of the height that can be less than or greater than the first angle, and less than or greater than the second angle). FIG. 22C illustrates an end view and FIG. 22D illustrates an isometric view of a raised feature 300 with a plurality of differing slopes and associated angles from the strip-facing base surface to the radially outward facing surface, where the angle θ2 between horizontal and an upward slope after a transition point can be greater than an angle θ1 between the horizontal strip-facing base edge and the intersecting upward slope (in other words, the first slope S1 from the strip-facing base edge base can be less steep than a second slope S2 higher up after a transition point). FIGS. 22E and 22F illustrate an embodiment of a raised feature 300 similar to FIGS. 22C and 22D except the angle θ2 can be less than the angle θ1 (in other words, the first slope S1 from the strip-facing base edge base can be steeper than a second slope S2 higher up after a transition point).

Alternately, some embodiments of the raised feature 300 may also include a series of steps at different heights where the width transitions to a narrower width and then continues to climb in height. When a series of steps is used in place of the bevel, it can sometimes be due to fabrication limitation when methods other than a reel of stainless steel is honed to an edge. For instances where chemical etching is used to form the steps from the strip-facing surface width W_{B} to the radially outward facing width Wu, the shape of the side walls of the raised feature 300 may not have a single slope. The side wall may tend towards a concave shape with one or more series of concave etch regions transitioning from the strip-facing surface width W_{B} to the radially outward facing width W_{U}.

The shapes of the radially outward facing edge or surface (e.g., radially outward facing surface 304 of FIG. 13) of the raised feature 300 can in some embodiments be the same height from one edge 306 of the radially outward facing length or width to the other edge 308. In some embodiments, the height along the radially outward facing surface 304 can vary from one edge 306 to the other edge 308. When the height of the radially outward facing edge or surface 304 varies, the radially outward facing edge can have a series of steps, spikes, or serrating elements. In some embodiments, the midpoint of the raised feature 300 along the radially outward facing length 304 between edges 306, 308 can be the highest point of the radially outward facing surface. However, in some embodiments, the highest point of the raised features 300 can be offset from the midpoint. In some embodiments, there may be a plurality of highest points interspersed by lower point relative to the bounded/base surface 302. The maximal variation of height between edges 306, 308 of the radially outward facing surface 304 of the raised features 300 and the radially outward facing surface 202 of the base strip 200 between the raised features 300 can in some embodiments be less than about 80%, 70%, 60%, 50%, 40%, 30%, 20%, 10%, or less than the total height of the raised feature 300. In some embodiments, the radially outward facing surface 202 of the base strip 200 facilities delivery of pulsatile energy of the pressure wave through the rigid material of the strip 200. In some embodiments, the radially outward facing surface 202 of the base strip 200 facilities delivery of pulsatile energy of the pressure wave through the shape of the strip 200.

In some embodiments, the base strip 200 has a roughened or otherwise textured inferior surface to aid in adhesion to an outer surface of the underlying balloon 100. The base strip can have any desired geometry such as square, rectangular, or in some embodiments trapezoidal with the bottom surface having a greater width, such as about or at least about 10%, 20%, 30%, 40%, 50%, 60%, 70%, 80%, 90%, 100%, or more of the top surface. In some embodiments between about ⅓ and ½ of the top surface of the strip 200 can be covered by raised features 300, while between about ½ and ⅔ of the top surface can be free of raised features 300. In some embodiments, the majority of the top surface of the strip 200 is free of raised features 300. The top surface of the strip 200 can contact the vessel wall in some embodiments. The top surface of the strip 200 can facilitate the hammering motion. The top surface of the strip 200 can hammer the surface of the vessel in the vicinity of the microperforations. The top surface of the strip 200 can hammer the surface of the vessel to facilitate longitudinal crack formation between the microperforations.

Referring to FIG. 14, in some embodiments, the radially outward facing surface of the raised feature 300 viewed from the top can be seen as a line extending from one edge of the radially outward facing length to the other edge of the radially outward facing length (e.g., where W_{U} can be a point assuming 310A is the radially outward facing surface of the device). This would be analogous to a honed or “razor-sharpened” edge with no apparent width. In some embodiments, the raised feature 300 can be honed. In some embodiments, the raised feature 300 can be sharpened into a cutting blade. In other embodiments, the top view can appear as an unhoned surface that is slightly blunt resembling a rectangle (e.g., if 310B or 310C is the top of the device, and assuming everything above those lines were cut off) with the width of the radially outward facing surface W_{U} being less than the strip-facing base surface W_{B} but directly correlated with the slope or slopes between the width edge and height from the strip-facing base surface to the radially outward facing surface. The raised features 300 can be micro wedges. In some embodiments, the top or the radially outward facing surface can be a line, a flat rectangle, a rounded or mounded surface (that might appear to be a rectangle or square in a 2-dimension point of view), or take a pyramidal, wedge, trapezoidal, or other polygonal shape. In some embodiments, the unhoned surface facilities delivery of pulsatile energy of the pressure wave through the raised feature 300.

In some embodiments, an unhoned width can be a width, for example, that can be about or a combination of about 1 nm, 5 nm, 10 nm, 50 nm, 100 nm, 500 nm, 1 μm, 2 μm, 5 μm, 7 μm, 10 μm, 15 μm, 20 μm, 25 μm, and 30 μm, measured at the radially outward facing edge or surface. In some embodiments, unhoned radially outward facing surfaces of the raised features 300 can be advantageous as being slightly blunt/relatively less sharp than honed edges, in situations for example where creating serrations, indentations, and/or microperforations in a target, for example, can be desirable rather than making cuts through the entire luminal wall. In some embodiments, the entire radially outward facing surface of the raised feature 300 has an unhoned width.

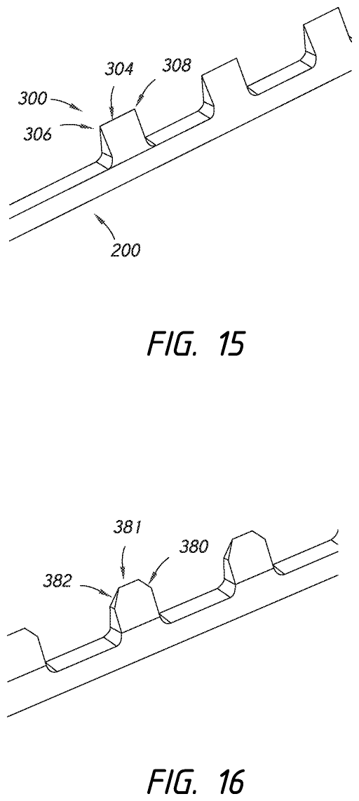

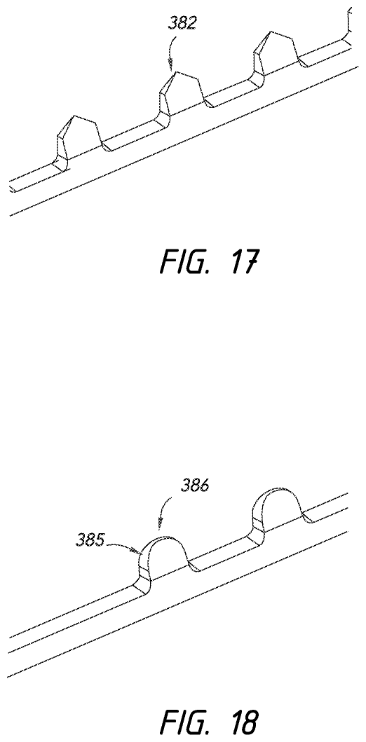

The shape of the raised feature 300 can take many forms, including further non-limiting embodiments as those shown in FIGS. 13-23D. For example, FIG. 15 illustrates the raised feature 300 rising from a base strip 200 with a honed or sharp radially outward facing surface 304 from edge 306 to edge 308. FIGS. 16-17 illustrates the raised feature 300 with chamfered segments 380 of a radially outward facing surface on both lateral edges that slope or otherwise ramp upward to a honed central single point 382 or edge having a length 381. The slope could be a straight line ramp, or follow a curve as seen in FIG. 18 below. As illustrated in FIG. 16, the raised feature 300 includes lateral segments 380 of radially outward facing surface that increases in height, but decreases in width from a first edge to a central mid-portion 381 having a length with minimal/negligible width, and then increases in width and decreases in width from the midpoint to the second edge. FIG. 17 illustrates a raised feature 300 similar to FIG. 16 except that the mid-portion is a single honed apex point 382. In some embodiments, the slope of the raised feature 300 facilitates delivery of pulsatile energy of the pressure wave through the raised feature 300.

FIG. 18 illustrates a raised feature 300 with a radiused radially outward facing surface 385 that increases in height from an edge along a first curved length but decreases in width from a first edge to a central zone such as a midpoint 386, then decreases in height and increases in width along a second curved length to another edge. In some embodiments, the radiused radially outward facing surface of the raised feature 300 can facilitates delivery of pulsatile energy of the pressure wave through the raised feature 300.

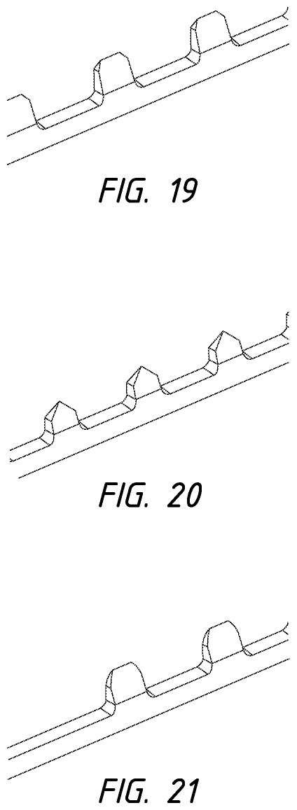

FIGS. 19-21 illustrate embodiments of raised features 300 with an unhoned, radially outward facing surface that do not include a sharp honed point or edge (e.g., having a width that is larger than that of a honed edge). FIG. 19 illustrates an embodiment of a raised feature 300 similar to that of FIG. 16, except the radially outward facing surface can be completely unhoned along its length. FIG. 20 illustrates an embodiment of a raised feature 300 similar to that of FIG. 17, except the radially outward facing surface can be completely unhoned along its length. FIG. 21 illustrates an embodiment of a raised feature 300 similar to that of FIG. 18, except the radially outward facing surface can be completely unhoned along its length. In some embodiments, the width of the radially outward facing surface of the raised feature 300 can facilitate delivery of pulsatile energy of the pressure wave through the raised feature 300.

One commonality of the embodiments of FIGS. 16-21 of the raised features 300 can be that the widths of the radially outward facing surfaces are greater (wider) at the lateral edges, and narrower/less wide more centrally, either at a central point or longer central segment. The height of the radially outward facing surface from one edge to the other edge can be arched or otherwise variable, e.g., with a highest point more centrally and the shortest height at one or more edges when viewed from the side. In these embodiments, the orientation of the narrowest or thinnest (least wide) section of the radially outward facing surface can be along the longitudinal axis of the strip 200, which may or may not be aligned with the longitudinal axis of the balloon 100. In some embodiments, the central segment of the radially outward facing surface of the raised feature 300 can facilitates delivery of pulsatile energy of the pressure wave through the raised feature 300.

In other embodiments, the narrower point or segment need not be symmetric about the midpoint of the length of the radially outward facing surface of the raised feature 300, but can be asymmetrical/offset from the midpoint of the length in some cases. In some embodiments, the offset shape of the raised feature 300 can facilitate delivery of pulsatile energy of the pressure wave through the raised feature 300.

Independent of the geometry of the raised features 300, some embodiments can be characterized by having a bounded end 302. The raised features 300 can have a base that the raised features 300 are attached to such as a spline, the strip 200, the balloon 100, the carrier 1200, and/or a molded element, The bounded end 302 can have a length and width. The raised features 300, in some embodiments, can be characterized by having a radially outward facing surface 304, with a length and width. In some embodiments, the width of the radially outward facing surface 304 can be about, or less than about 90%, 85%, 80%, 75%, 70%, 65%, 60%, 55%, 50%, 45%, 40%, 35%, 30%, 25%, 20%, or less than the width of the strip-facing bounded end 302, or ranges incorporating any of two of the foregoing values. The width of the strip-facing bounded end 302 of the raised feature 300 (as well as the width of the strip 200) can be fixed or constant along the length of the strip 200. The width of the strip-facing bounded end 302 of the raised feature 300 (as well as the width of the strip 200) can be variable in some embodiments. In some embodiments, the connection between the strip 200 and the raised feature 300 can facilitates delivery of pulsatile energy of the pressure wave through the raised feature 300.

The raised features 300 can be a number of different sizes and shapes. In some embodiments, the raised features 300 can be about or less than about, for example, 0.10″, 0.09″, 0.08″, 0.07″, 0.06″, 0.05″, 0.04″, 0.03″, 0.02″, or 0.01″ in length at the strip-facing base end or ranges incorporating any of two of the foregoing values, or between about 0.01″ and about 0.06″, or between about 0.01″ and about 0.04″ in length. In some embodiments, the raised features 300 can be about or less than about 0.05″, 0.04″, 0.03″, 0.025″, 0.02″, 0.015″, 0.01″, or 0.005″ in height as measured from the unbonded edge of the base strip, or between about 0.005″ and about 0.025″ or between about 0.01″ and about 0.025″, or between about 0.005″ and about 0.015″ in some embodiments. In some embodiments, the dimensions of the raised feature 300 can facilitate delivery of pulsatile energy of the pressure wave through the raised feature 300.

The raised features 300 can be micro wedges. The raised features 300 can, in some embodiments, have a strip-facing base length of about, or less than about 25 mm, 20 mm, 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm long, or ranges incorporating any two or more of the foregoing values. In some embodiments the raised features 300 have a strip-facing base length of 2 mm, 2.5 mm, or 3 mm long, or between about 1 mm and about 5 mm long, or between about 1.5 mm and about 3.5 mm long. The raised features 300 can be spaced apart in a regular or irregular fashion to increase the flexibility of the device. For example, the space between adjacent raised features 300 can be, for example, between about 2 times to about 10 times the strip-facing base length of the raised features 300, with the raised features 300 positioned lengthwise. For example, in some embodiments, raised features 300 with a strip-facing base length about 2.5 mm long can have about 5 mm spaces between them, or about 25 mm spaces between them. In some embodiments, groups of raised features 300 can be spaced apart with a first smaller ratio of, for example, about 1-4 times the strip-facing base length of the raised features 300 and then a group of raised features 300 can be spaced apart by a second larger ratio, for example, about 8-10 times the strip-facing base length of the raised features 300. For example, a first group of raised features 300 with a strip-facing base length of 2.5 mm can have 5 mm spaces between adjacent raised features 300 and then a second group of raised features 300 can be spaced 20 mm from first group. The second group of the raised features 300 can have the same or a different size, shape, and or spacing as the first group of raised features. In some embodiments, the orientation of the raised feature 300 relative to the strip 200 can facilitate delivery of pulsatile energy of the pressure wave through the raised feature 300.

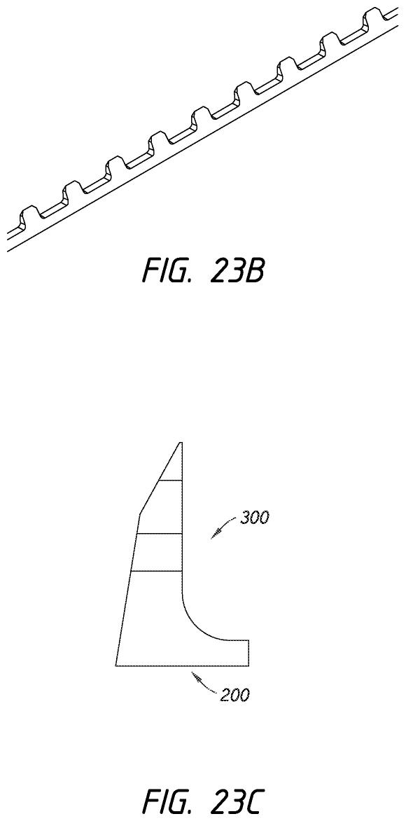

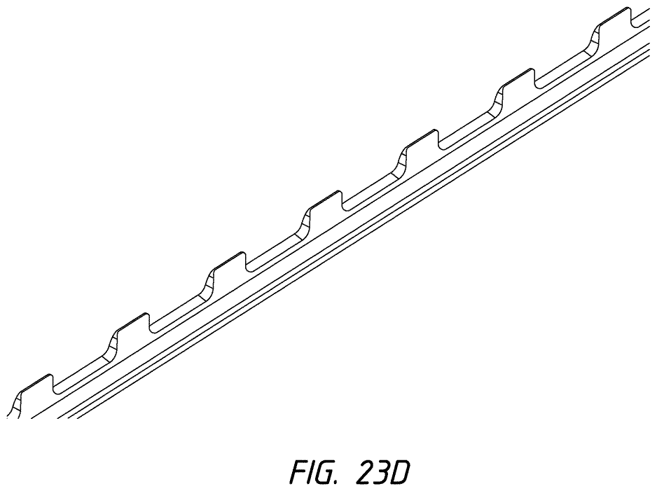

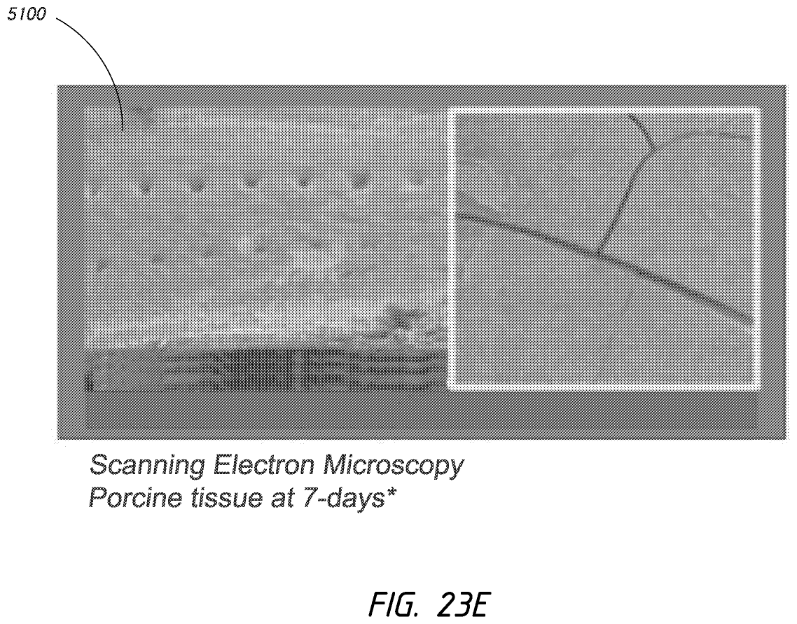

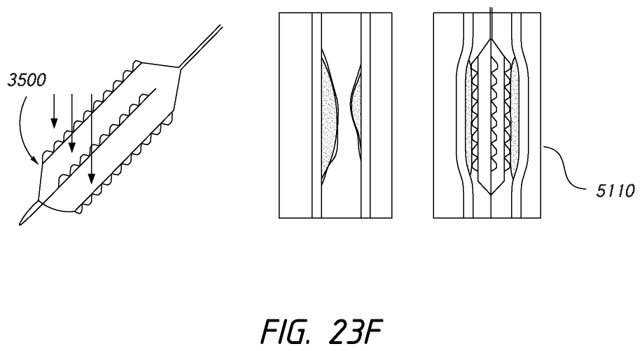

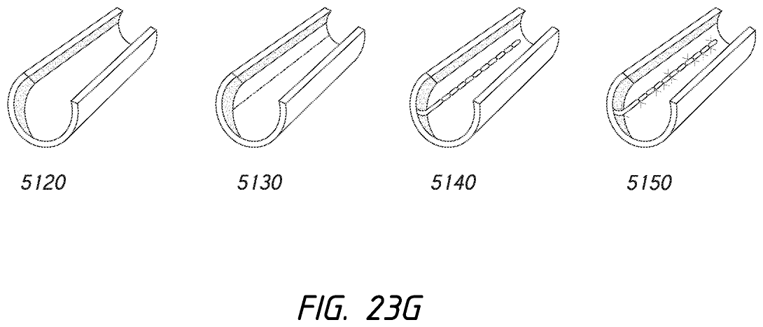

The location of the radially outward facing surface relative to the strip-facing base surface of the raised features 300 may not always centered or symmetric in some embodiments. In other words, the midpoint of the radially outward facing surface can be offset from the midpoint of the strip-facing base surface. FIGS. 23A-23B illustrate an asymmetric radially outward facing surface of the raised feature 300. An asymmetric radially outward facing surface can be off center with respect to the alignment of a radially outward facing width edge directly over the strip-facing base width edge. In this embodiment, only one of the strip-facing base width edges has a tilted edge 340 climbing in height off of the radially outward facing surface while the other height edge 342 can be perpendicular, at a 90 degree (right) angle RA to the strip-facing base surface 344, as shown in FIG. 23A. In addition, the edges of the radially outward facing surface in one or both of the width ends and/or in one or both of the length ends can be chamfered or beveled or have a radius. In some embodiments, the radially outward facing surface location can be limited to the area projected upward over the strip-facing base surface. The radially outward facing surface can be a sharp line (e.g., honed edge) or any of the described unhoned edge variations for example. FIGS. 23C-23D illustrate an embodiment of the raised feature 300 where the total volume or substantially the total volume of the raised feature 300 rises or is present over less than the entire width (or surface area) of the base of the strip 200, such as about or less than about 70%, 60%, 50%, 40%, or 30% of the width or surface area of the strip 200. The raised features 300 can be asymmetrically offset either anteriorly or posteriorly from the longitudinal axis of the strip 200. In some embodiments, the geometry of the raised feature 300 relative to the strip 200 can facilitates delivery of pulsatile energy of the pressure wave through the raised feature 300.

In some embodiments, radially outward facing surface 304 of the raised feature 300 may have a varying height (increasing from first height at first edge 306 to second height at second edge 308) from the strip-facing base surface 302 and may include edge profiles that can be rounded with a radius of curvature of the radially outward facing length edges 306, 308. In some embodiments, the height of the raised feature 300 can facilitate delivery of pulsatile energy of the pressure wave through the raised feature 300. In some embodiments, the edge profile of the raised feature 300 can facilitate delivery of pulsatile energy of the pressure have through the raised feature 300.

In some embodiments, the various raised features 300 described herein can offer advantages to aid in delivery of the device, including but not limited to reducing vessel trauma if the radially outward facing surface can be positioned outside of the delivery apparatus and/or can contact the luminal wall and has the potential to scrape the vessel wall during movement through the artery. This can be the case, for example, in embodiments with raised features with unhoned, radially outward facing surfaces.

In addition, not to be limited by theory, certain shapes may offer more effective penetration into the tissue. For instance, raised features 300 that include chamfered or rounded radially outward facing edges can potentially enter the vessel wall with less force (requires less pressure to penetrate tissue) while still weakening the tissue and enabling tissue expansion with minimal vessel trauma and cellular injury.

Furthermore, while there have been proposals for providing blades or sharp edges or scoring wire on a balloon during angioplasty or other procedure for cutting or scoring the plaque in conjunction with balloon expansion, these methods can have problems or disadvantages which are eliminated or avoided by systems and methods as disclosed herein.

Cutting or scoring a luminal wall, such as, for example, the plaque during angioplasty can be performed at high pressures that can result in high injury to the blood vessel. The cutting blades, edges or scoring wire can be forced into the wall of the blood vessel at the same time that the angioplasty balloon can be expanded to dilate the plaque. During this process the cutting blades, edges, or scoring wire can be forced into the vessel wall at oblique angles and can plow up the plaque potentially increasing the tendency for dissections and the potential need for implants like stents. In contrast, in some embodiments, the raised features 300 can be expanded into the plaque at low pressures so as to form precise microperforations, serrations, and/or indentations in a radially outward direction that form precise indentations, cleavage lines or planes in the plaque or other location in the luminal wall, or other target. The radially outward facing surface of the raised feature 300 can push into the plaque or other luminal surface in small surface areas, thereby being much less likely to plow up the plaque or luminal surface.

The raised features 300 can be designed, in some embodiments, to provide a series of oriented punctures, initiation sites, or serrations into (but not completely through in some cases) a diseased vessel wall, which can create in some cases predictable and controlled lumen expansion along the serrated lines with minimal injury. In some embodiments, the raised features 300 can create serrations without cutting with blades with honed or sharp edges. The perforations can serve as a pathway such as micro-channels for pharmaceutical or other agents as shown in FIG. 23E. The pharmaceutical or other agents could be delivered using a drug-coated balloon, incorporated either with the device disclosed herein, or on a separate device that can be used following the usage of the disclosed device. In some embodiments, the raised features 300 can be detachable from the strip 200. In some embodiments, the raised features 300 be coated or otherwise impregnated with one or more pharmaceutical agents for drug delivery. The raised features 300 can produce a linear line of weakness or perforations without cutting a continuous axial segment of the vessel wall. The raised features 300 can enable more effective and gentler vessel lumen expansion 5110 as shown in FIGS. 23F and 23G. These figures provide examples of stages of gradual expansion and serration in 5110, 5130, 5140, 5150. The balloon 100 can be inflated and while the pressure in the balloon 100 increases the following series of events can occur: the balloon unfolds in the artery and the strips 200 can be exposed from their resting place within the folds; the tips (e.g., radially outward facing surface) of the raised features 300 on the strips 200 can contact the wall; the tips' relatively narrow profile can penetrate the wall generating nucleation sites for the fissuring event; the fissures quickly produce cracking along the intra-luminal surface; due to the proximity and alignment of the cracks, the cracks join to become a long crack along the intra-luminal surface that can extend along the entire length of the strip 200, or less than the strip length, or greater than the strip length; and/or the depth of the penetration of the crack can be similar to the depth of medial tissue. In some embodiments, pressure waves generated by the generator 800 can accelerate intercellular fracturing by splicing and thinning fibrin strands. Fibrin strands can be fibrous proteins that can be a major component of atherosclerotic plaque. In some embodiments, the pressure waves break the fibrin strands. The broken fibrin bands enable greater access and exposure pathways into the tissue. The pathways can be for the delivery of healthy blood and/or medication through direct balloon delivery or system flow. These pathways that extend into the medial tissue provide drug receptor sites with greater surface area for absorption and retention.

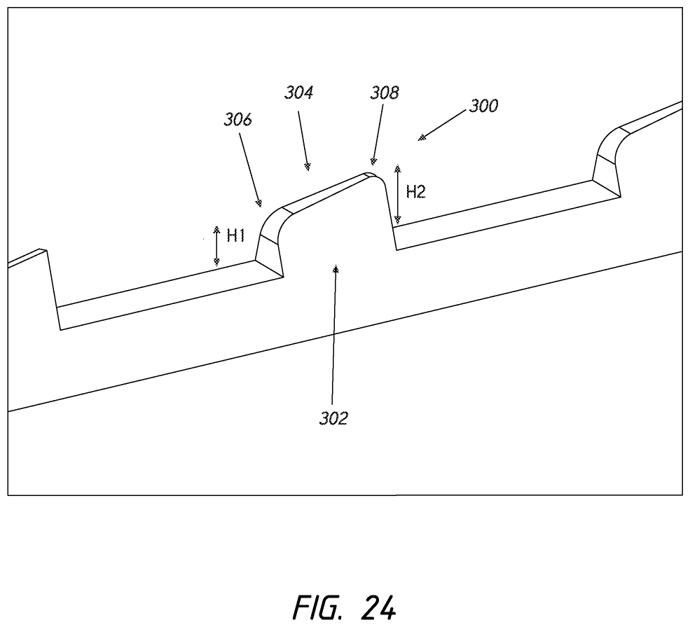

FIG. 24 illustrates an embodiment illustrating how the radially outward facing surface 304 of the raised feature 300 can have a varying height (increasing from first height H1 at first edge 306 to second height H2 at second edge 308) from the strip-facing base surface 302. The outward facing surface 304 can include edge profiles that can be rounded with a radius of curvature of the radially outward facing length edges 306, 308. The outward facing surface 304 can include a wider radius of curvature at one edge 306 that has a shallow height H1 measured from the strip-facing base surface 302 while the radius of curvature of the opposite edge 308 can be narrower and has a longer height H2 measured from the strip-facing base surface 302.

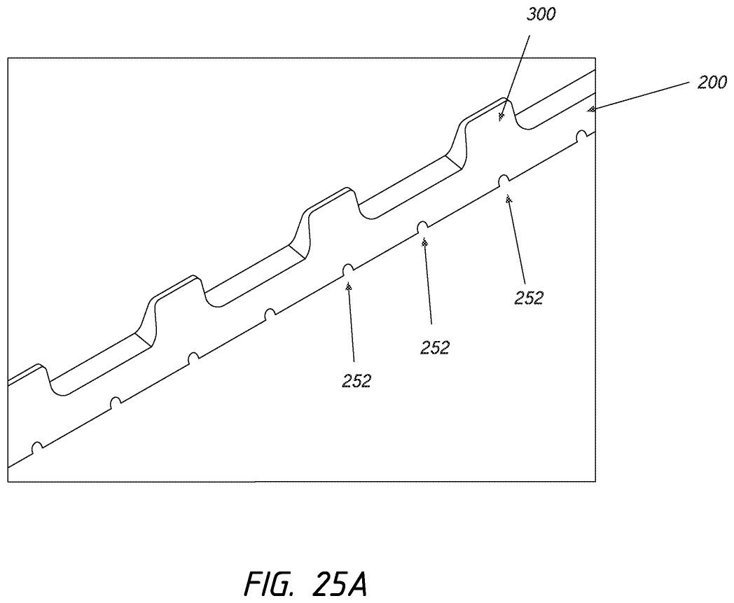

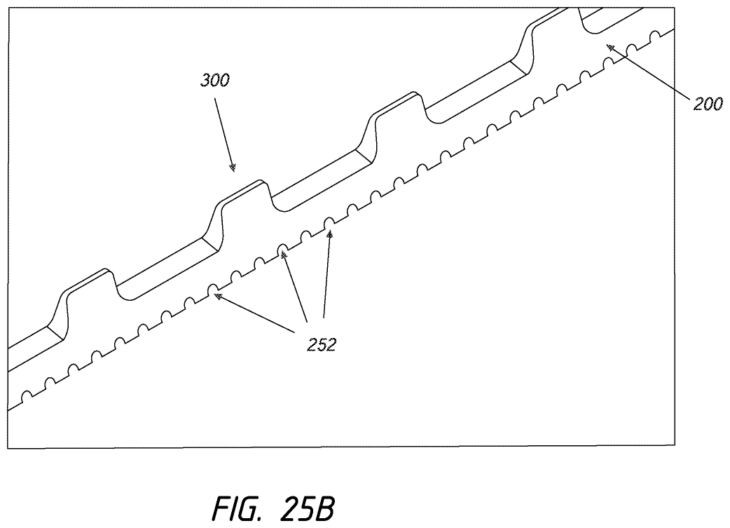

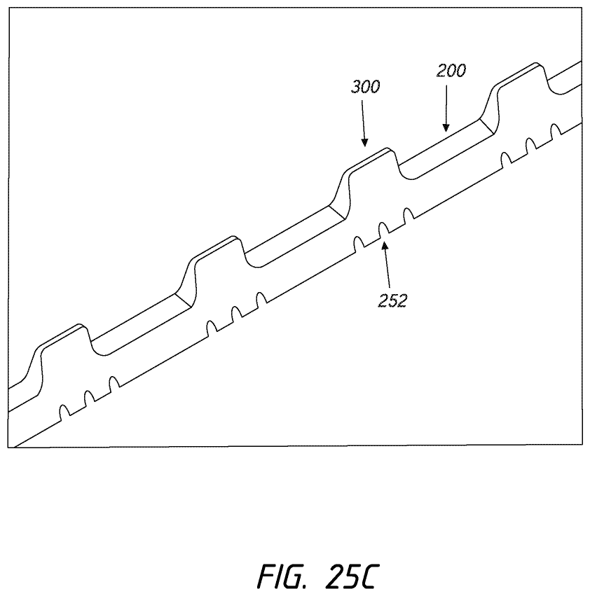

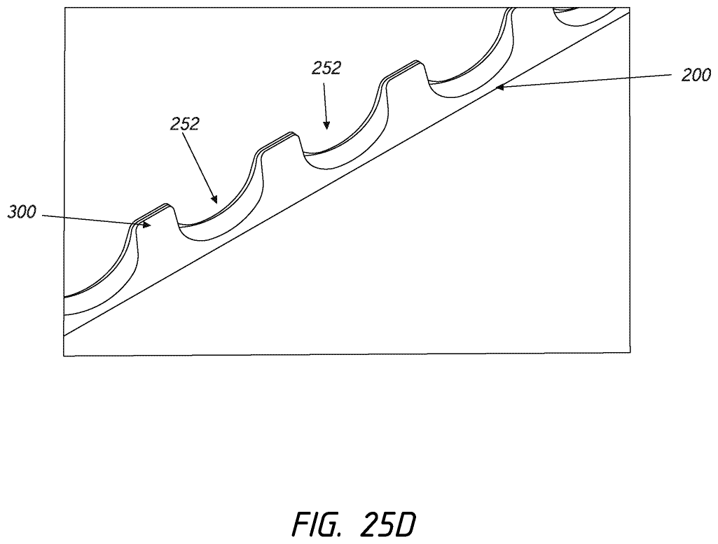

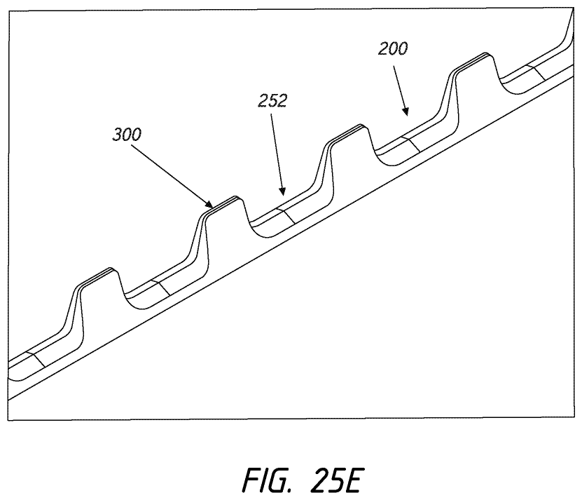

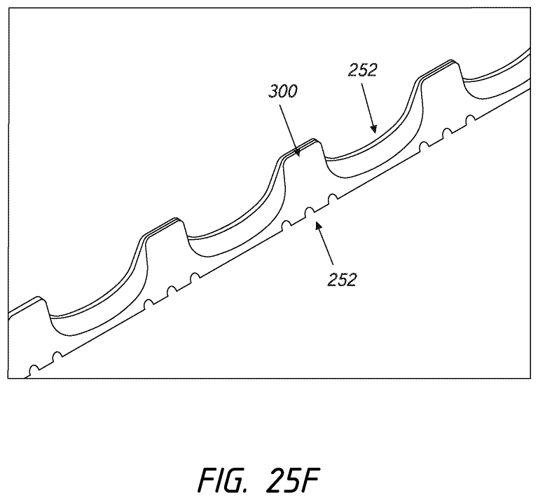

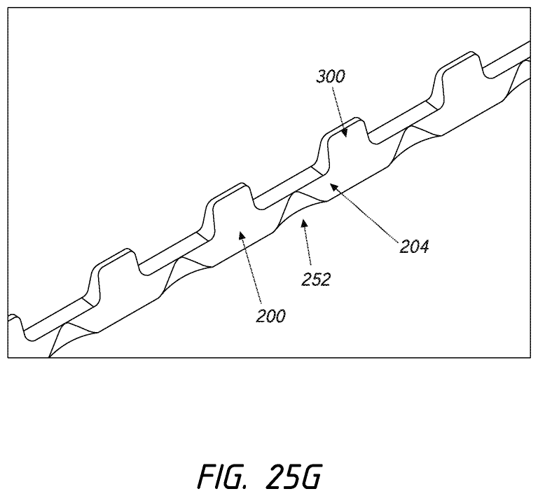

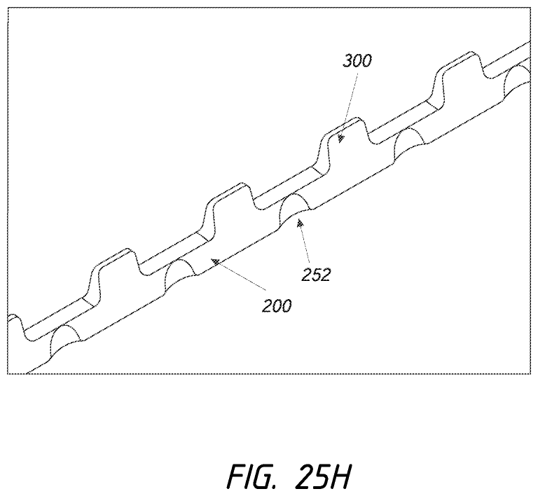

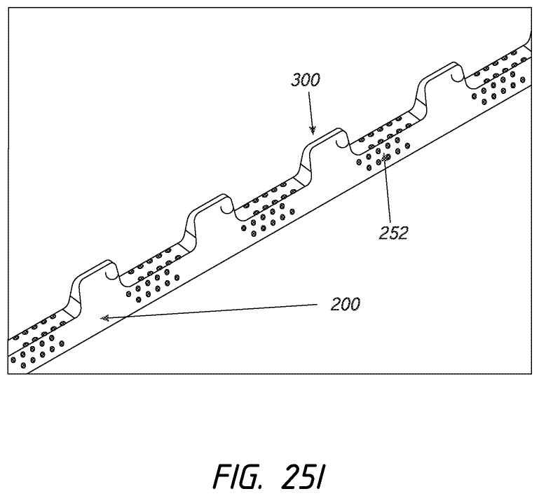

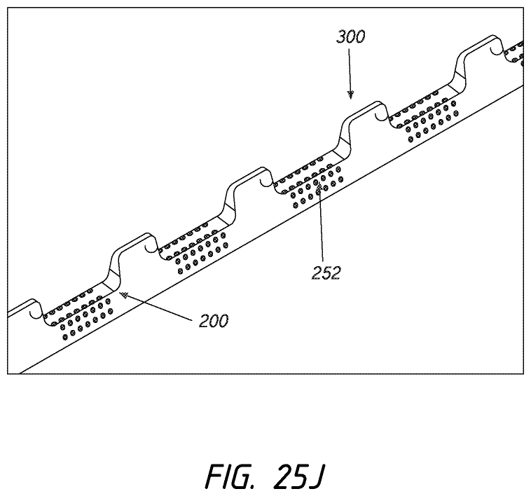

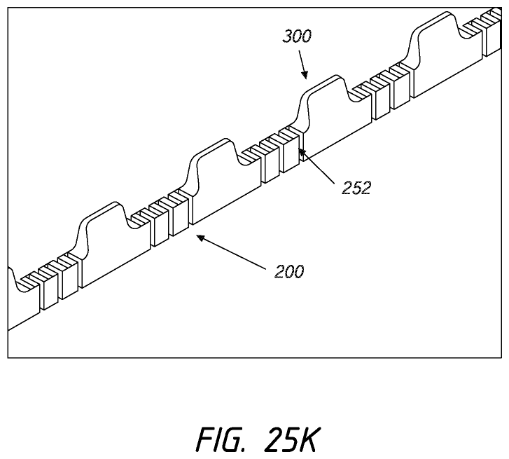

To reduce potential rigidity of strip 200, a series of reliefs on the strip 200 can be added in some embodiments, as illustrated in FIGS. 25A-25K. The relief elements can be produced to have material removed. The reliefs can provide a more pliable strip 200 for the raised features 300 to be attached. Relief can be made in the base of the strip 200 opposite the strip-facing base surface 302 of the raised feature 300, at the top of the strip 200 directly adjacent the strip-facing base surface 302 of the raised feature 300, or in both locations, e.g., a combination of top and bottom of the strip 200. The reliefs can also be made on the side of the strip 200. In some embodiments, through apertures can be added to the strip 200. Any combination of top, bottom, side or through apertures can be added to the strip 200 to offer relief. In some embodiments, the reliefs of the strip 200 can facilitate delivery of pulsatile energy of the pressure wave through the strip 200.

In some embodiments, the strip 200 can have relief holes or slits located at the top, bottom, centered or off center that can be either circular, rectangular, linear, triangular, or elliptical or combinations thereof. The strip 200 offers a supporting base infrastructure, intended to be flexible and follow the movement of the balloon 100, for the raised features 300 to be oriented correctly.

The relief holes illustrations can be specifically configured to offer a pathway for balloon-based pharmacological agents to migrate through; in addition, they offer strain relief in the surface to enhance the deliverability of the device in tortuous anatomy. FIGS. 25A-25C illustrate embodiments of raised features 300 with reliefs 252 on the inferior surface of the strips 200 opposite the bounded surface of the raised features 300. FIG. 25A illustrates an embodiment where the reliefs 252 can be regularly spaced apart by approximately a length of the bounded surface of each raised feature 300. FIG. 25B illustrates an embodiment where the reliefs 252 can be regularly spaced apart by 50% or less of the length of the bounded surface of each raised feature 300. FIG. 25C illustrates an embodiment where each relief 252 can be spaced apart by 50% or less of the length of the bounded surface of each raised feature 300, but the reliefs 252 can be grouped only under the raised features 300 and are not present under the strip sections in between the raised features. In other embodiments, the reliefs 252 can be grouped only under the strip sections in between the raised features 300, but not under the strip sections directly below the raised features 300.

FIGS. 25D-25E illustrates an embodiment where the reliefs 252 can be present on the superior-facing surface 202 of the strip 200 in between the raised features 300. In FIGS. 25D and 25E, the reliefs 252 form depressions in the superior-facing surface 202 of the strips 200 in between raised features 300 with a generally curved based as illustrated in FIG. 25D, and a relatively more square or rectangular base as illustrated in FIG. 25E, with or without rounded edges. FIG. 25F is an embodiment combining two different kinds of reliefs 252 found in the embodiments of FIGS. 25C and 25D. Other permutations of combinations can be also possible, depending on the desired clinical result. FIGS. 25G and 25H illustrate other embodiments where the reliefs 252 can be on an anterior side surface 204 and/or posterior side surface 204 of the strip 200. FIG. 25G illustrates generally pyramidal-shaped reliefs 252, while FIG. 25H illustrates generally arcuate reliefs 252. The reliefs 252 can be spaced axially apart from the raised features 300 as shown, and/or spaced axially aligned with raised features 300 in other embodiments. FIGS. 25I and 25J illustrate embodiments where the reliefs 252 take the form of vertically (FIG. 25I) or horizontally (FIG. 25J) oriented through-channels, which can be spaced axially apart from the raised features 300. In some embodiments, the reliefs 252 can be oriented at an oblique angle to the longitudinal axis of the strip 200. FIG. 25K illustrates an embodiment where the reliefs 252 take the form of slots on the anterior and/or posterior side surfaces, which can be bounded at the base surface and/or at other locations.

In some embodiments, the longitudinal axis of the strips 200 can be longitudinally oriented along the balloon 100 and spaced apart from each other. The strips 200 can be spaced apart circumferentially. The strips 200 can be unconnected from other strips at proximal ends of the strips 200. The strips 200 can be unconnected from other strips along the lengths of the strips 200. The strips 200 can be unconnected from other strips at distal ends of the strips 200. The strips 200 can be equally spaced around the circumference of the balloon 100. The strips 200 can be unequally spaced around the circumference of the balloon 100. The strips 200 can be separated by lobes 120 of the balloon as described herein. The strips 200 can be separated by lobes 120 having equal dimensions. The strips 200 can be separated by equal parts of the balloon 100. The strips 200 can be disposed along the periphery of the balloon 100. The strips 200 can be separate from each other. The strips 200 can be detached from each other. The strips 200 can unattached from other strips 200. The strips 200 can independent. The strips 200 can be uncoupled for independent movement. The strips 200 can isolated from the movement of other strips. In some embodiments, the strips 200 do not completely cover the circumference of the balloon 100.

While not to be limited by a theory, there can be benefits of having the strips 200 unconnected from other strips when used in conjunction with pressure waves. The unconnected strips 200 can undulate as the pressure wave propagates. The unconnected strips 200 can raise and lower each raised feature 300 from the proximal end to the distal end of the strip 200. The unconnected strips 200 can move independently. The unconnected strips 200 can allow the lobes 120 of the balloon 100 to directly contact the vessel. It can be significantly easier to transmit acoustic waves, including acoustic waves with ultrasonic frequencies, when surfaces are in direct contact compared to when the surfaces are separated. The lobes 120 of the balloon 100 can be directly contacting the vessel wall when transmitting the acoustic waves. The lobes 120 can touch the calcified lesion. The lobes 120 can form a continuous path for energy. The lobes 120 can allow the acoustic wave to travel from one solid, the balloon 100, to another solid, the calcified lesion. The acoustic impedance matches better from solid surface to solid surface, than solid surface to air. When a wave travels from one solid to another solid through direct contact, there can be reduced impedance mismatch. The lobes 120 of the balloon 100 can impart the pressure wave directly to the vessel. The lobes 120 can impart strain on the vessel wall to propagate cracks. The lobes 120 can impart shearing stress on the calcium by delivering the pressure wave. The lobes 120 can have a large surface area for the acoustic wave to travel through. The lobes 120 can be in direct contact with the vessel due to the unconnected nature of the strips 200. There can be longitudinally orientated structures in contact with the vessel. The strip 200 can directly contact the vessel. The raised features 300 can directly contact the vessel. It can be significantly easier to transmit acoustic waves when surfaces are in direct contact compared to when the surfaces are separated. The raised features 300 can be directly contacting the vessel wall when transmitting the acoustic waves. The raised features 300 can touch the calcified lesion. The raised features 300 can form a continuous path for energy. The raised features 300 can allow the acoustic wave to travel from one solid, the strip 200 with the raised features 300, to another solid, the calcified lesion. The acoustic impedance matches better from solid surface to solid surface, than solid surface to air. When a wave travels from one solid to another solid through direct contact, there can be reduced impedance mismatch. The raised features 300 can impart the pressure wave directly to the vessel. The raised features 300 can impart a force to create a nucleation site. The raised features 300 can impart shearing stress on the calcium by delivering the pressure wave. The raised features 300 can have a small surface area for the acoustic wave to travel through compared with the strip 200, thereby concentrating the wave. The raised features 300 can be in direct contact with the vessel.

While not to be bound by a theory, the longitudinally orientated structures can facilitate the cracks to propagate longitudinally when pressure waves are generated. The raised features 300 can amplify the pressure along longitudinally oriented serrations. The pressure wave can be concentrated along the raised features 300 to deliver more energy. The cleavage planes can be created faster with pressure wave generation. The cleavage planes can extend generally longitudinally. The cleavage planes can be caused by weaknesses in the anatomical structure or lattice of the calcium. The cleavage planes can be generated faster by applying localized energy along the raised features 300. The longitudinally oriented raised features 300 can exploit pre-existing weak planes in calcium, causing the calcium to fracture along the lines. The raised features 300 of the plurality of strips 200 can create parallel lines. The raised features 300 of the plurality of strips 200 can create lines that segment the lesion. The cleavage lines can occur because the atomic bonds in certain planes can be weaker than other planes. The calcified lesion can preferentially split along longitudinal lines due to the unconnected nature of the strips 200 with the raised features 300. The strips 200 with the raised features 300 can be spaced apart circumferentially to minimize circumferentially cracking. The raised features 300 can be oriented with a longer length than width to facilitate the longitudinally cracking. These and other features of the balloon 100, the strip 200, and the raised features 300, when used in conjunction with the pressure waves, can produce unexpected results.

In some embodiments, the strips 200 do not completely cover the length of the balloon 100. For example, in one embodiment an 80 mm long balloon 100 can have strips 200 that measure 76.6 mm. For example, in one embodiment an 80 mm long balloon 100 can have a series of strips 200 measuring less than 80 mm. While the length of the strip 200 can be the same as the defined working balloon length, in some embodiments the length of the strip 200 can be shorter than the defined working balloon length to allow for balloon contraction that can be typically observed when a balloon 100 goes to rated burst pressure. The length of each strip 200 can in some cases be no more than about 15%, 10%, 9%, 8%, 7%, 6%, 5%, 4%, 3%, 2%, or 1%, or between about 1% and about 7%, between about 1% and about 5%, or between about 1% and about 4% shorter than the overall working balloon length. In some embodiments, the working balloon length does not include the lengths of the cones. In some embodiments, the working balloon length corresponds to the body of the balloon.

In some embodiments, part of the strip 200, e.g., the base of the strip (e.g., the inferior-facing surface 203 configured to be attached to the outer surface of the balloon 100) can be roughened to aid in adhesion. The inferior-facing surface 203 can be attached to the balloon 100 in any manufacturing methods described herein. The strips 200 can be attached to the base balloon 102 of the balloon 100. The strips 200 can be retained by the outer retention balloon 104 of the balloon 100. The outer retention balloon 104 can cover at least a portion of the radially outward facing surface 202 of the base strip 200 between the raised features 300. The strip 200 can be coupled to the balloon 100 in any manufacturing methods described herein.

The raised features 300 can be fabricated in many different manufacturing methods and in a large range of shapes. Regarding the manufacturing processes, the strip 200 and/or the raised features 300 may be fabricated using one or more additive or subtractive processes. Additive processes such as high energy vapor deposition, for instance laser chemical vapor deposition, self-assembly techniques, polymer/metal 3D printing, selective laser sintering, powder printers, or other stereo lithographic can be a few such options but other additive processes may be used to manufacture the raised features 300 and/or the strip 200. Alternatively, subtractive processes can be used to manufacture the raised features 300 and/or the strip 200 such as etching, CNC milling, laser cutting, water jet, or electrical discharge machining can be just a few examples, but other subtractive processes may be used.

In some embodiments, a method of fabrication of the raised features and/or the strip 200 includes the use of a reel of martensitic stainless steel, such as for example a 300 or 400 series stainless steel with a hardness of as low as 48 and as high as 70 but more typically between about 52 to about 64 on the Rockwell C-scale (HRC) or a broader Mohs range between 4 and 7, although other materials can be used. The reel can be either honed or precisely unhoned on one or both sides of one edge of the steel to form the raised features 300. In some embodiments, the steel can be in the form of a thin reel strip between about 0.004″ and about 0.020″ thick or between about 0.005″ to about 0.010″ thick, and can be as wide as the processing system can handle. Sometimes this can be as large or larger than 36″.wide. The edge that can be either honed or unhoned can be a single hone or two or more honed angles to form the raised features 300. In some embodiments, when the angle of a honed edge can be measured as the slope from the bounded end to the height of the unbounded end, the angle of the honed edge can be, for example, greater than about 75 degrees. But when more than one honed angle is used, then the tip angle can be less than, for example, about 75 degrees. In some embodiments, when a honed edge is measured as the angle between the strip-facing surface width W_{B} to the radially outward facing width Wu, the unhoned edge can have an angle of about, at least about, or an amount between 70, 75, 80, 85 degrees or greater as it moves toward the honed edge in a series of bevels. In addition to the honed edge, independent of the number of honed angles, in some embodiments a separate and additional edge can be generated at the very tip of the unbound edge of the strips 200. When added, the additional tip edge height from the honed edge to the unbounded edge can be very short and typically has a much larger angle than the overall honed edge. Independent of the number of honed angles used, the unbounded tip width, Wu, can be described as the radius of the tip. The unbounded tip width, W_{U} can be the penetrating edge into the lesion, when the width is, in some cases, less than about 0.015″ or 0.006″, the surface area of the raised feature 300 can be minimized to have a less pronounced contact surface with the vessel enabling a reduced amount of energy requirement for penetration of the raised feature 3000. When the raised feature 300 is configured for penetration into harder surfaces such as calcium beds, in some cases either a more obtuse angle or the removal of the unbound tip at a greater distance from the unbounded surface can produce a wider tip edge (see FIG. 21, Wu). Not to be limited by theory, this wider edge of the raised feature 300 distributes the load across the larger surface area generating a more effective resistance to tip deformation when the raised feature 300 is pressured into rigid tissue surfaces. Once the reel is sharpened, the reel can be stamped to a desired length of the raised features 300. In some embodiments, the reel can be hardened and then stamped to the desired length. Independent of when the stamping occurs, the raised features 300 can in some cases be passivated and hardened above, e.g., about HRC 45. but more typically in a range of from about HRC 58 to about HRC 62. The hardened raised features can then be laser cut, stamped, EDM'ed or another precise metal shaping technology to form the shape of the raised feature 300 as described herein. In some cases, the raised features 300 can be processed on the reel and then hardened and passivated. In some embodiments of strips 200 where the raised feature 300 is not a sharpened honed edge, the tip of the raised feature 300 that was produced during the reel sharpening step can be removed during the raised feature and strip manufacturing step to produce an unhoned edge. In some cases, the material removal (711) can be configured to start a distance, such as from about 0.0001″ to about 0.003″ below the honed edge (710), or from about 0.0001″ to about 0.0005″ can be removed from the honed edge (710), producing a flat top (Wu) as illustrated in FIG. 21. The thinnest edge remaining (now a flat top in some cases) on the previously honed edge side can be what will become the unbounded surface of the raised feature 300.

In some embodiments, disclosed are methods for attaching the strips 200 to the balloon 100. The methods can include any number of processing steps that provide effective strip retention, perpendicular orientation, and structural stability during the fabrication and use. In one embodiment the inferior-facing surface 203 of the strip 200 can be coated with a base coat of an appropriate material, such as a polymer, e.g., polyurethane through a controlled dipping process producing a uniform layer of polyurethane. The coating can be dried and typically 3 or 4 strips are aligned with a strip alignment mechanism or jig and glued with a medical grade cyanoacrylate into place at predetermined orientations on the balloon 100. The number of strips 200 and the periodicity can vary from, for example, 1 to 8, for example 1 strip, 2 strips, 3 strips, 4 strips, 5 strips, 6 strips, 7 strips, 8 strips, between 1 and 3 strips, between 2 and 4 strips, or any range of two of the foregoing values. In some embodiments, the balloon 100 does not include any strips. The number of strips 200 can be associated with the same number of balloon folds for example, 1 to 8 folds. The number of strips 200 can be less than the number of folds and the periodicity can be non-sequential. Once the strips 200 are bonded to the balloon 100, a single or series of multiple top coats or retention layers, can be placed over the raised features 300 to retain the strips 200 and protect the balloon 100 from the thin tips of the raised features 300. In some embodiments, these retention layers follow a similar process as the base or pre coat using a controlled dipping process producing one or more uniform layers of urethane or polyurethane. In some embodiments, there can be no base coat and only one top coat. Variations in the numbers of base coats and top coats can be between 0-4 on either base coat or the top coat. Once the retention layer or layers are cured, a layer of hydrophilic or other coating may be applied to decrease balloon friction and increase the balloon's deliverability and retrievability. When incorporated, the outer slip coating can increase the functionality of the balloon 100 by reducing the force to insert and retract the device. In some embodiments, the balloon 100 can include the base balloon 102 and the outer retention balloon 104. The inferior-facing surface 203 of the strip 200 can be attached to the base balloon 102. In some embodiments, the retention balloon 104 can be placed over the raised features 300. The raised features 300 can be interrupted scoring elements disposed on the strip 200.

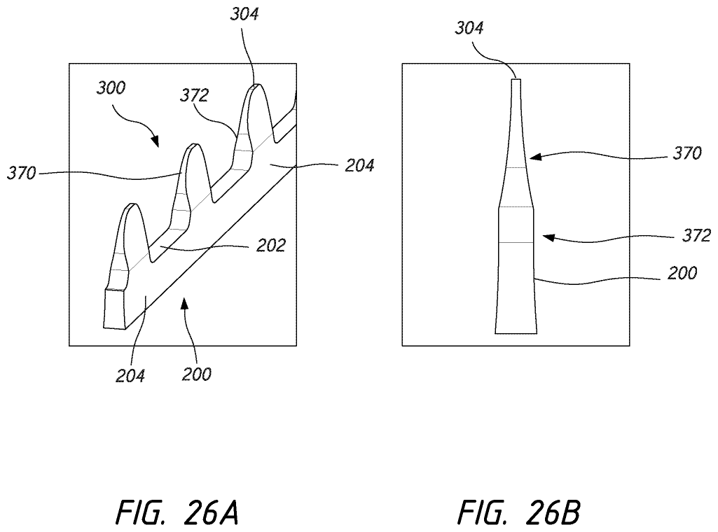

FIG. 26A illustrates an embodiment of a strip 200 with raised features 300 where the raised feature 300 has sloped non-linear edges. The raised features 300 can include radially-outward facing peaks 360. The strip 200 can has the top surface 202 and the side surface 204. The sloped non-linear edge of the raised features 300 can have an upper concave or convex feature 370 that can be different from the bottom concave or convex feature 372 along one or both sloped side surfaces 204 of the strip 200. Another view can be seen in the FIG. 26B end view illustration. The raised features 300 can have one or more concave features. The raised features 300 can have one or more convex features. The strip 200 can have one or more concave features. The strip 200 can have one or more convex features.

In the side view shown in FIG. 26B, the bottom feature 372 can be a biconcave section of the strip 200 that has a greater thickness than the upper feature 370 which can be a biconcave section of the raised feature 300. The upper feature 370 shows a thinner biconcave section as the strip 200 and/or the raised feature 300 narrows towards the radially outward-facing surface 304 in some cases. Other designs and manufacturing techniques can produce a series of concave sections on the side surfaces 204 of the strip 200. In some embodiments, the concavity of the raised feature 300 can facilitate delivery of pulsatile energy of the pressure wave through the raised feature 300. In some embodiments, the concavity of the strip 200 can facilitate delivery of pulsatile energy of the pressure wave through the strip 200.

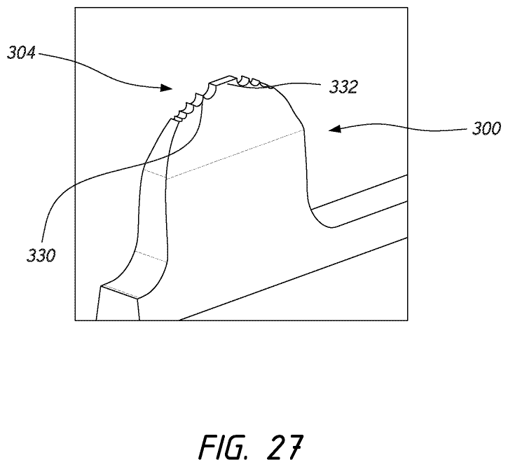

As illustrated in FIG. 27, the top of the raised feature 300 can have a variety of features 330 on the radially outward facing surface 304 that contacts the tissue. As shown, the raised feature 300 has the appearance of a serrated tip with micro-concavities, valleys, or features 330 near the apex of the radially-outward surface of the raised feature 300, that have small, e.g., less than about 10%, 8%, 6%, 4%, 2%, or less increases or decreases in height with respect to the entire height of the raised feature 300 that laterally border a central segment 332 which can be flat-topped as shown. In some embodiments, microcavities of the raised feature 300 can facilitate delivery of pulsatile energy of the pressure wave through the raised feature 300.

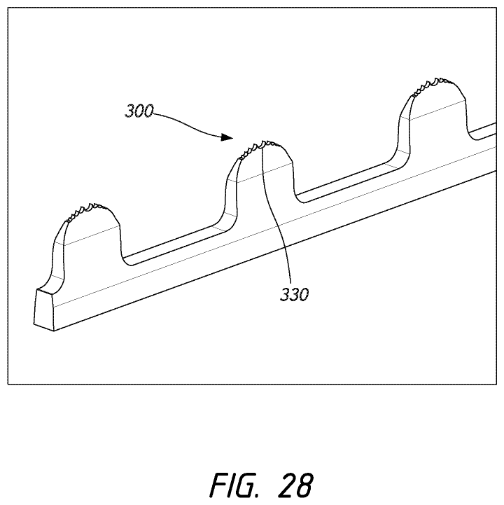

FIG. 28 is another design illustrating an alternate variation of the serrated edge of the raised feature 300, where the central segment can include a small depression 330 as shown, which can have a depth of, for example, less than about 10%, 8%, 6%, 4%, 2%, or less increases or decreases in height with respect to the entire height of the raised feature 300. In some embodiments, the shape of the raised feature 300 can facilitate delivery of pulsatile energy of the pressure wave through the raised feature 300.

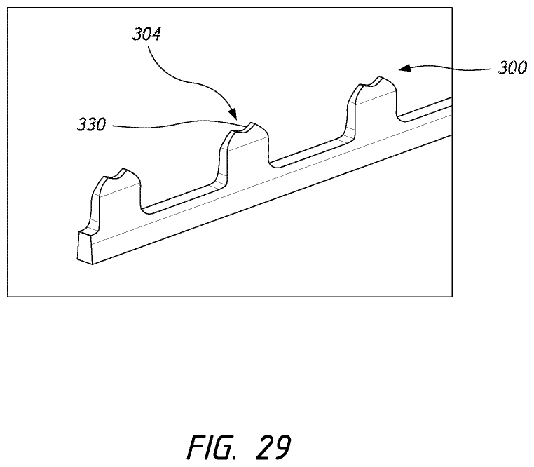

In another embodiment, shown in FIG. 29, the raised features 300 can include rounded double-hump like contacting surfaces 330 at the radially outward facing surface 304 that can provide effective tissue penetration. The effect of multiple points of contact on the surface of the raised feature 300 can in some cases provide better penetration force (point source) into a variety of disease morphologies, including calcium, while still enabling serration effect across the diseased lesion and thereby facilitating arterial lumen gain with minimal balloon pressure. The shape of the contacting surfaces of the raised features 300 can facilitate delivery of pulsatile energy of the pressure wave through the raised feature 300.

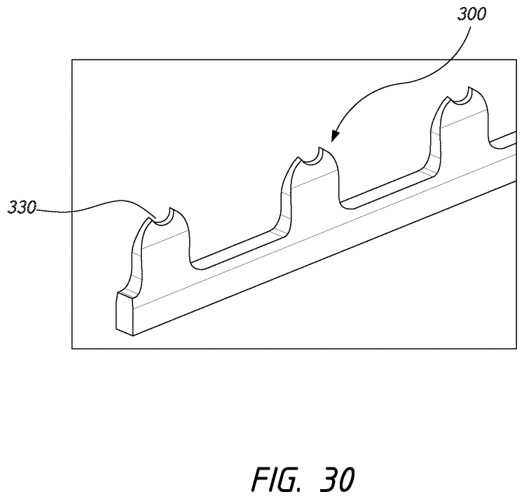

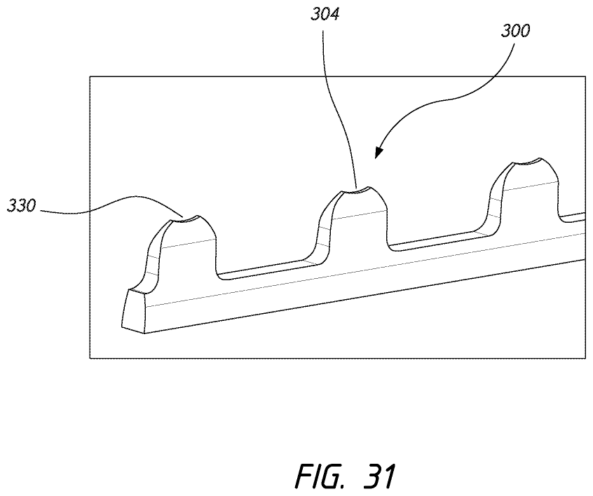

FIG. 30 illustrates variations on a design that provides a relatively sharp, pointed double contacting surface surfaces 330 at the radially outward facing surface 304 of each raised feature 300 that can provide effective tissue penetration. FIG. 31 also illustrates a similar design that provides a relatively sharp, pointed double contacting surface at the tip of each raised feature 300 that can provide effective tissue penetration, that abut a central wider, and shallower valley/depression respectively. The shape of the contacting surfaces of the raised features 300 can facilitate delivery of pulsatile energy of the pressure wave through the raised feature 300.

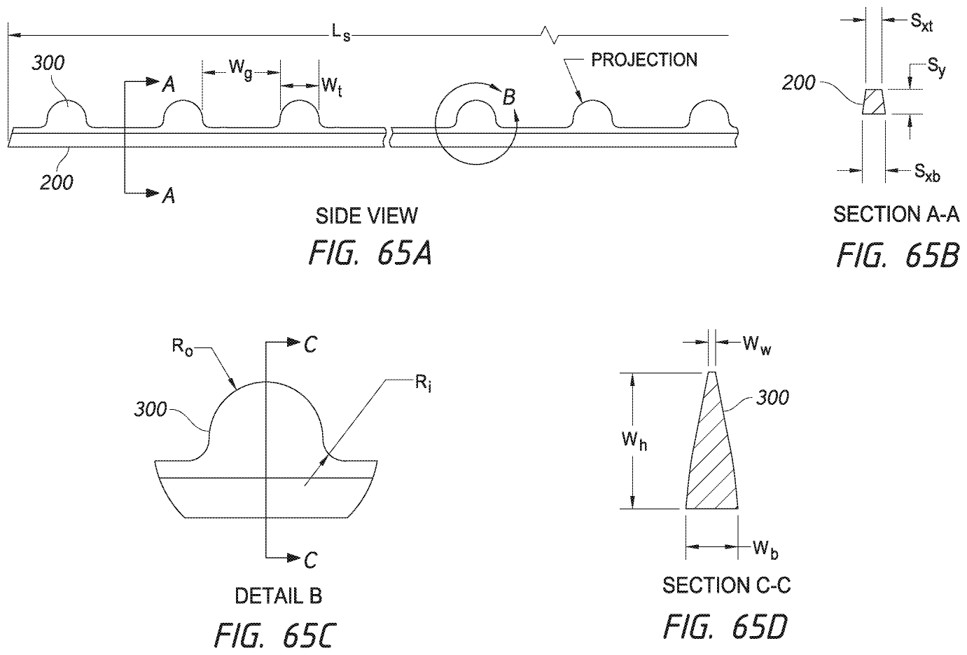

The balloon 200 with raised features 300 can include various dimensions as shown in FIGS. 65A-65D. The overall strip length Ls can range from 5 mm to 300 mm with lengths directly correlated to the overall balloon length. The projection width Wt can range from 0.1 mm to 0.8 mm. The gap between projections, Wg, can range from 0.1 to 2.0 mm. The strut region, shown in Section A-A can have a base width Sxb which can range from 0.05 mm to 1.0 mm but can be more typically between 0.07 mm to 0.25 mm, a height Sy which can range from 0.01 mm to 0.5 mm but can be more typically between 0.1 mm to 0.2 mm, and a top width Sxt, which can range from 0.001 mm to 0.07 mm but can be more typically between 0.07 mm to 0.25 mm. The projection can have radii features Ro which can range from 0.001 mm to 0.5 mm but can be more typically between 0.07 mm to 0.25 mm and Ri which can range from 0.001 mm to 0.5 mm but can be more typically between 0.05 mm to 0.13 mm. The projection cross section, shown in Section C-C, can have a base width Wb of 0.04 mm to 0.5 mm but can be more typically between 0.07 mm to 0.20 mm, height Wh can have a range of 0.1 mm to 2.5 mm but can be more typically between 0.2 mm to 0.4 mm, and top width WW can have a range of 0.0005 mm to 0.1 mm but can be more typically between 0.002 mm to 0.06 mm. Additional features of the balloon, strip, and raised features can be found in U.S. patent Ser. No. 10/166,374, issued Jan. 1, 2019, which is incorporated by reference herein in its entirety. Additional features of the systems can be found in U.S. Pub. No. 2022/0211983 published Jul. 7, 2022, U.S. Pub. No. 2024/0001092 published Jan. 4, 2024, and U.S. Pub. No. 2025/0213828 published Jul. 3, 2025, which are incorporated by reference herein in its entirety.

The strip material selection can be limited to those compatible with blood contact (e.g. Nitinol, certain grades of stainless steel, and synthetic polymers such as polyether ether ketone (PEEK)). Material property selection can be influenced by: (1) stiffness, (2) bonding capability (i.e. surface adhesion properties), (3) compatibility with fabrication processes (e.g. stamping, grinding, laser cutting, chemical etching, EDM, etc), (4) fatigue resistance, (5) hardness, and/or (6) cost.

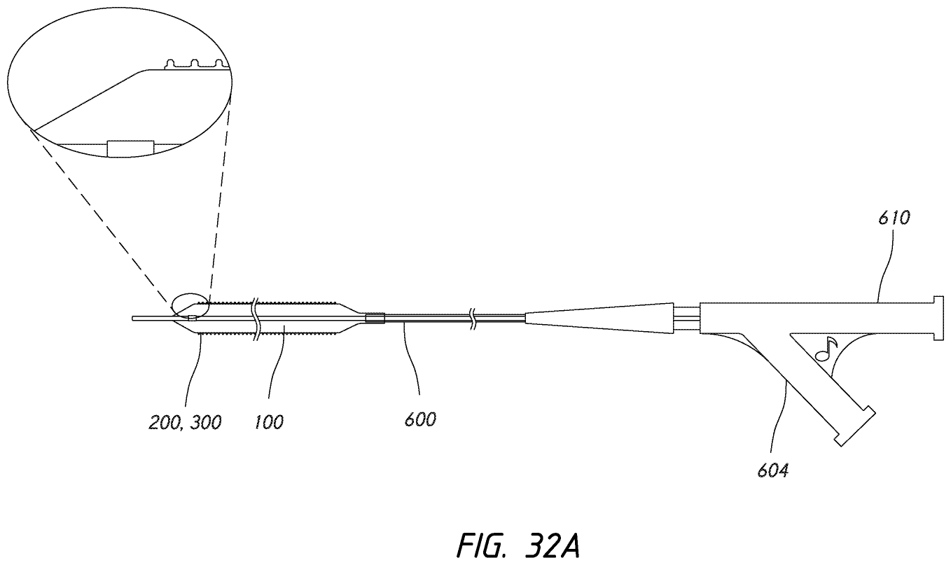

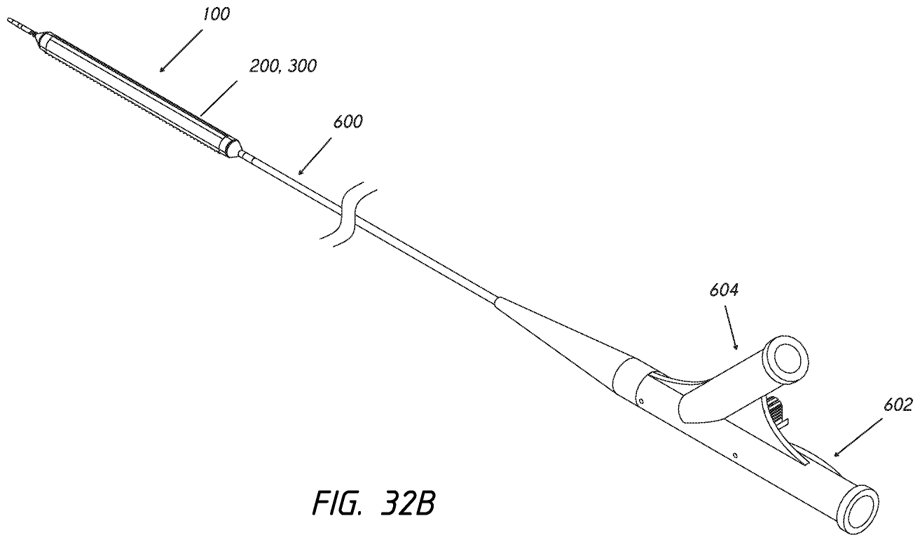

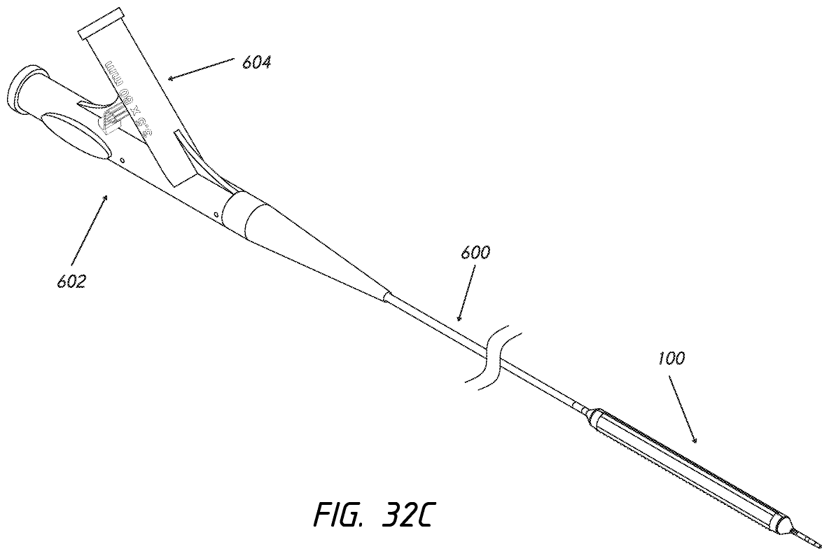

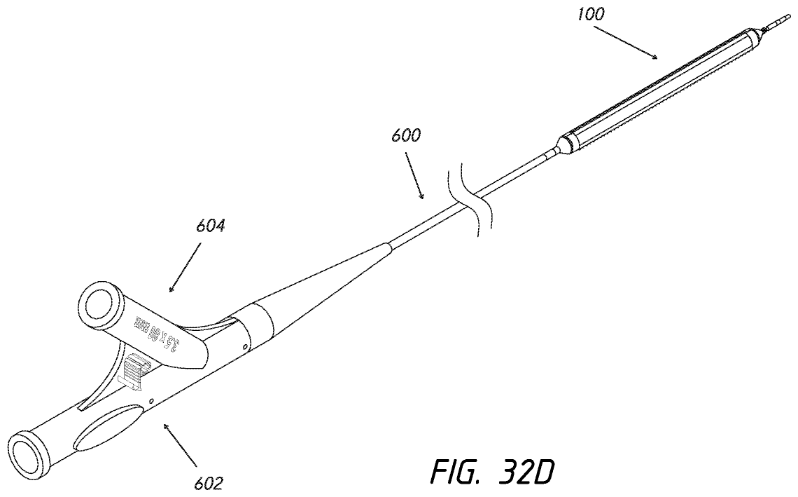

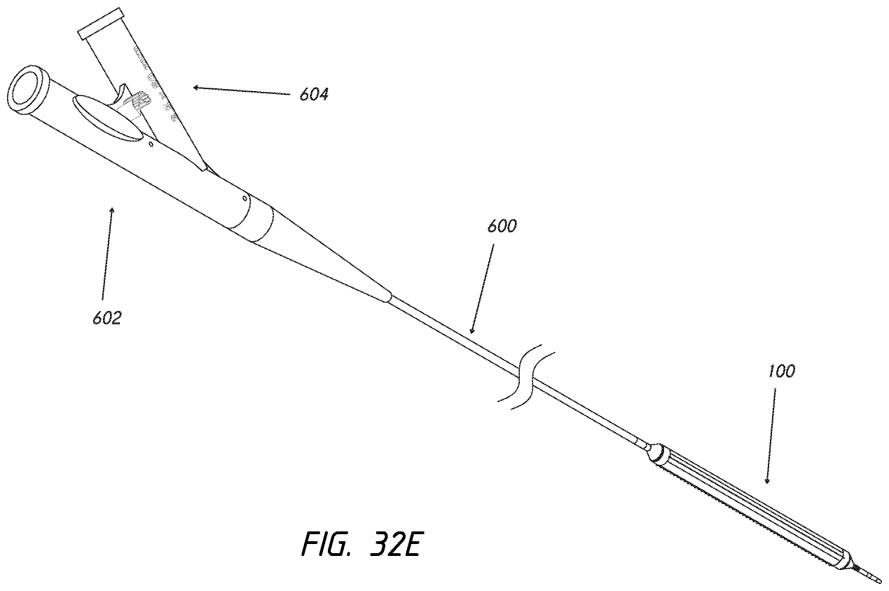

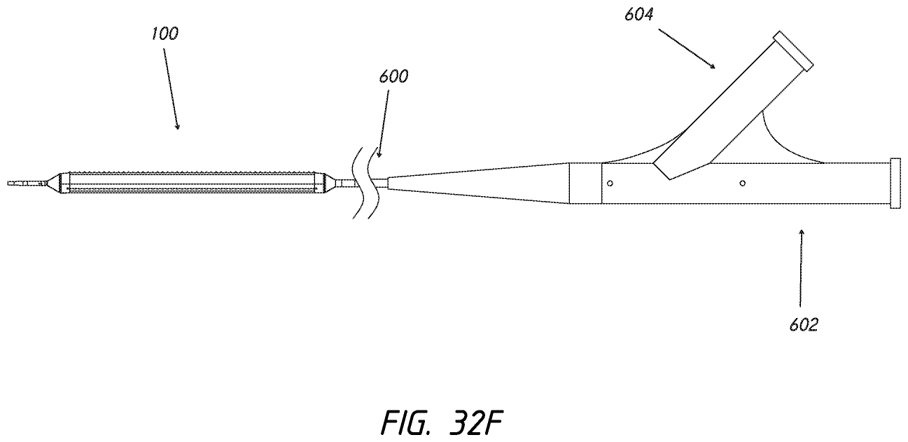

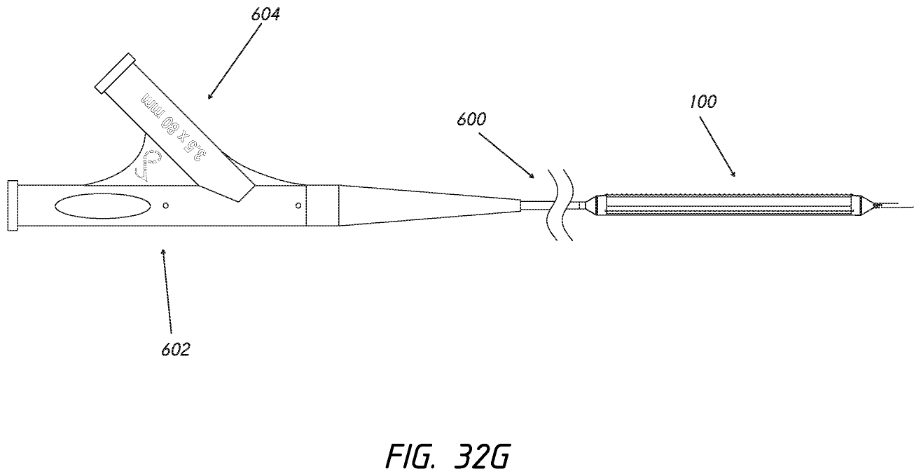

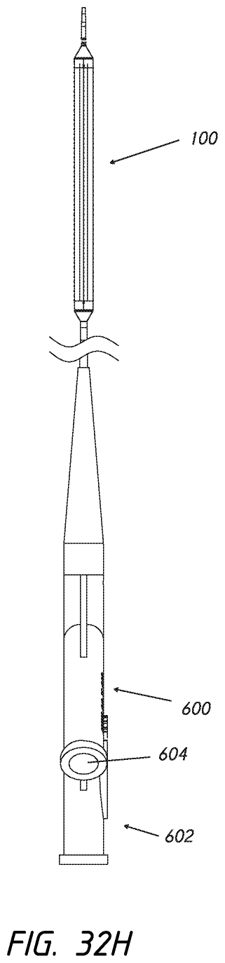

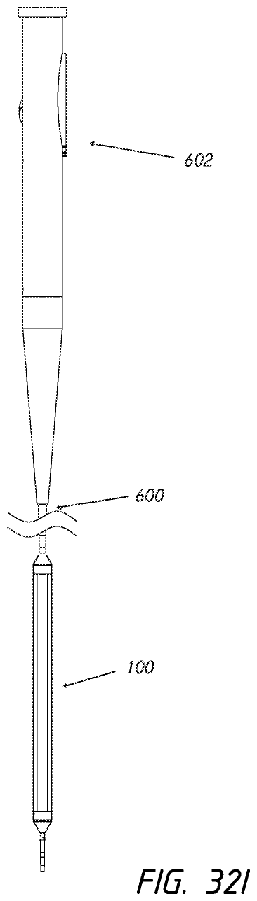

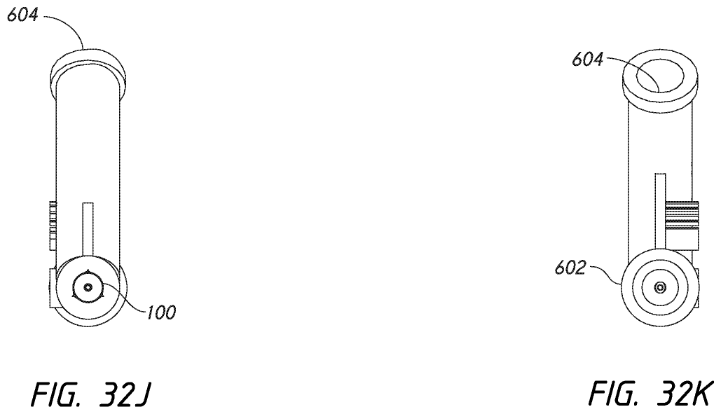

FIG. 32A is an illustration of one embodiment of an overall system for producing serratoplasty showing a series of serrating raised features 300 on the outer diameter of the balloon 100 attached to a catheter 600 with a guidewire hub 610 and the balloon inflation hub 604. The balloon inflation hub 604 can include features to couple with the inflation device 400. The proximal handle of the system can include features to couple with the generator 500. The proximal handle can include the generator 800 as described herein. FIGS. 32B-32J illustrate additional views of the system 1000.

In some embodiments, the raised features 300 described herein can offer advantages to aid in delivery of the pressure wave. The raised features 300 can have the radially outward facing surface 304 with a small surface area relative to the strip 200. The raised features 300 can magnify energy delivery as the pressure wave travels through the strip 200 to the raised features 300. The raised features 300 can have sloped sidewalls that direct the pressure wave. The raised features 300 can have a blunt surface which delivers the pressure wave. The pressure wave can be focused outward by the raised features 300. The pressure wave can be focused by the small surface area of the radially outward facing surface 304 of the raised features 300. The pressure wave can travel longitudinally along an edge of the raised features 300. The pressure wave can travel longitudinally as directed by the long edge of the radially outward facing surface 304 of the raised features 300. The pressure wave can travel longitudinally as directed the longitudinally oriented raised features 300. The pressure wave can travel longitudinally as directed the longitudinally oriented strip 200. The pressure wave can travel transversely along an edge of the raised features 300. The pressure wave can travel transversely as directed by the short edge of the radially outward facing surface 304 of the raised features 300. The pressure wave can travel transversely as directed the raised features 300. The pressure wave can travel transversely as directed the strip 200. In some embodiments, the cleavage planes created by the pressure waves do not intersect other cleavage planes. In some embodiments, the cleavage planes created by the pressure waves are only longitudinally oriented.

The raised features 300 of the strips 200 can be manufactured by any methods described herein. In some embodiments, chemical etching can be used. In some embodiments, the strips 200 including the raised features 300, or a portion thereof, can be fabricated in many different manufacturing methods. The strips 100 including the raised features 300 can be fabricated using one or more additive or subtractive processes. Additive processes such as high energy vapor deposition, for instance laser chemical vapor deposition, self-assembly techniques, polymer/metal 3D printing, selective laser sintering, powder printers, or other stereo lithographic can be a few such options but other additive processes may be used. In some embodiments, the strips 200 including the raised features 300, or a portion thereof, can be fabricated by subtractive processes such as chemical etching, CNC milling, stamping, laser cutting, water jet, pulse electro chemical machining, or electrical discharge machining can be just a few examples but other subtractive processes may be used. In some embodiments, the strip 200 with the raised features 300 can be laser cut, stamped, EDM'ed, pECM'ed, or another precise metal shaping technology to create the strips.

In some embodiments, chemical etching can include some or all of the followings steps. A mask or mask set can be created for the pattern of the strip 200 that includes the details related to the size and shape of the raised features 300. The mask can be placed on a photo resistant layer and each mask comprises a series of openings to allow light to shine through the mask. The mask set can allow partial etching through of a single side of the stainless steel material. Chemical etching of material such as stainless steel sheets using masks, photoresist, and etching materials can be advantageously applied to allow for large volumes of material to be etched at low costs. Bulk chemical etching can allow for extremely repeatable and low cost parts to be fabricated in volume. Traditionally, chemical etching produces rounded edges with gentle slope side walls through the material at angles approximating 90 degrees. To achieve more gentle sloped angles, new masking techniques can utilize relatively narrow patterns to control etch rates. By controlling the etch material flow through the resist layer, angles for the strip 200 with raised features 300 have been achieved. Two-sided mask exposure can enable etching through the material from both sides. With dual side exposure the edge profile produces greater control mirror imaging profiles on either side of the stainless-steel material. The chemical etching can form the strip-facing base surface 302 to the radially outward facing surface 304. The chemical etching can form the shape of the side walls of the raised features 300 to have one or more slopes. The side walls of the raised features 300 may tend towards a concave shape with one or more series of concave etch regions transitioning from the strip-facing base surface 302 to the radially outward facing surface 304. The lateral surfaces 314, 316 of the raised feature 300 from the strip-facing base surface 302 to the proximal edge of the radially outward facing surface 304 can have one or more a parabolic slope as would be generated by chemical etching. The lateral surfaces 314, 316 of the raised feature 300 from the strip-facing base surface 302 to the distal edge of the radially outward facing surface 304 can have one or more a parabolic slope as would be generated by chemical etching.

Other methods like electrochemical machining or other low-cost high volume can be envisioned for manufacturing the strip 200 with the raised features 300. The bulk processing can include one or more processes such as chemical etching, pulse electro chemical machining (PECM), electric discharge machining (EDM), CNC milling, CNC drilling, CNC machining, CNC laser machining, CNC plasma cutting machining, water jet cutting, polygon machining, milling, precision stamping, or other bulk processing used alone or in combination. Incorporated alone, or with the bulk processing, some methods can include extrusions, stamping, progressing die stamping, deep drawn metal stamping, transfer stamping, multi-side metal stamping, cutting, punching, blanking, embossing, piercing, bending, coining, embossing, flanging, precision stamping, and or forming, and or shaping to produce the intended shape. In some embodiments, the manufacturing method can use materials that start in a flat stock pattern, or flat stock reel, and can be post processed and reshaped to resemble the intended design. The flat stock can be shaped with either one or two sides or edges. The edges can be bent or lifted to an angled orientation relative to the base section. The edges can be perpendicular to the base section or with an angle pointing inward or outward to some degree. The edges can become the radially outward facing surface 304 that can be intended to create prick points in the plaque or vessel wall. In some embodiments, the intended final design of the strip 200 can include a series of raised features 300 along the length of the strip 200.

In some embodiments, a method of fabrication of the strip 200 with the raised features 300 can include the use of a flat sheet or reel of metal such as stainless steel. The metal can be martensitic metal such as martensitic stainless steel. The steel can be, for example, a 300 or 400 series stainless steel with a hardness of about 52 to about 64 on the Rockwell C-scale (HRC) although other materials can be used. In some embodiments, the material can be a polymer. The pattern of the strip 200 can be formed from the material. The pattern can include one or more edges that form the raised feature 300. In some embodiments, the pattern of the strip 200 can be about 0.007″ to about 0.015″ thick and between about 0.25″ to about 0.75″ wide, but can range between about 0.005″ and 0.020″ thick and between 0.15″ and 1″ wide. The material can be tapered, single side etched, and/or flattened toward the edges of the pattern. In some embodiments, the material retains a width such that the edge or edges remains unhoned. In some embodiments, the edges may not be honed or “razor-sharpened” or an edge with no apparent width. The strips 200 can be passivated and hardened above, e.g., about HRC 45, but more typically in a range of from about HRC 58 to about HRC 62.

In some embodiments, the strips 200 described herein comprise a series of raised features 300. In some embodiments, the strips 200 when initially produced can include a base section with a pairing of periodic features on either side of the base section that can be shaped to produce raised features 300. In some embodiments, once produced, the edges of the pairing of periodic features be tilted approximately 90 degrees. The shape of the raised features 300 can take many forms, including further non-limiting embodiments as described herein. In some embodiments, the raised features 300 rise from a base section with an unhoned radially outward facing surface 304. In some embodiments, the raised features 300 can include chamfered segments. In some embodiments, the raised features 300 can be tilted inward after placement relative to the base balloon 102 and the outer retention balloon 104. The raised features 300 can include surfaces that slope or otherwise tilt inward to the radially outward facing surface 304. The slope could be a straight slope, or follow a curve, or have one or more sloped segments. The raised features 300 can be radiused along the length. The raised features 300 can follow a smooth curve. The raised features 300 can increase in height to a midpoint and then decrease in height. The raised features 300 can have the radially outward facing surface 304 that does not include a sharp honed point or edge. The raised features 304 can have the radially outward facing surface 304 having a width that can be larger than that of a honed edge. In some embodiments, the radially outward facing surface 304 can be formed by a separation of two or more edges. In some embodiments, the radially outward facing surface 304 can be formed by two or more edges by folding relative to each other.

The raised features 300 could be surface treated, such as with plasma. In some embodiments, the addition of a surface functionalization can be applied to the strip 200. The functionalization can be done in a large plasma chamber under vacuum conditions with a plurality of strips 200 placed within the plasma field. The deposited functionalized layer can be very thin in the order of hundreds of angstroms. The use of surface enhancement or functionalization can produce an effective bonding surface. The plasma can be deposited on the entire surface of the strip 200. The plasma can be deposited on one or more surfaces of the strip 200. The plasma layer can facilitate bonding between the strip 200 and the balloon 100. The plasma layer can facilitate bonding between the strip 200 and adhesive. The plasma layer can facilitate bonding between the strip 200 and the outer retention balloon 104. The plasma layer can improve the bonding coefficient between two surfaces, such as the surface of the strip 200 and another layer.

The methods described herein can be used to manufacture various strips 200 with raised features. The systems can include any number of strips, such as one strip, two strips, three strips, four strips, five strips, six strips, seven strips, eight strips, nine strips, ten strips, twenty strips, thirty strips, or any range of two of the foregoing values. The strip 200 can include one raised feature 300. The strip 200 can include a plurality of raised features 300. The strip 200 can include one or more raised features 300 with a blunt or unhoned radially outwardly facing surface 304. The strip 200 can include one or more raised features 300 with a sharpened or honed surface. The methods described herein can be used to attach the strip 200 to the balloon 100. The methods described herein can be used to attach the strip 200 to the outer retention balloon 104. The methods described herein can be used for raised feature 300 of the strip 200 to penetrate the outer retention balloon 104. The methods described herein can be used for the raised feature 300 of the strip 200 to press through holes of the outer retention balloon 104. The methods described herein can be used to attach portions of strips 200 together to form the carrier 1200. The methods described herein can be used to bend the strip 200 into a final shape. The disclosure relates to the products formed by the processes described herein.

### Carrier

The systems and methods can be utilized with the carrier 1200 as shown in FIG. 61. The carrier 1200 can be configured for expansion within the vessel. The carrier 1200 can be any surface to carry the raised features described herein. The balloon 100 can be configured for sonic generation. The balloon 100 can be folded to be inserted into the carrier 1200. The generator 800 can be a source for energy for the carrier 1200. The balloon 1000 can be attached to the generator 800, and inserted into the carrier 1220 and generate energy through the balloon into the carrier 1200. The carrier 1200 can be used in conjunction with the pressure waves generated by the generator 800. The carrier 1200 can be a temporary stent. The carrier 1200 can be a permeant stent. The balloon 100 can be used with the carrier 1200. The balloon 100 can deploy the carrier 1200 in a balloon-assist deployment. The carrier 1200 can include the strip 200. The carrier 1200 can include any features of the strip 200 described herein. The carrier 1200 can omit the strip 200. The carrier 1200 can be a cage. The carrier 1200 can be an expandable structure. The carrier 1200 can be a stent. The carrier 1200 can be a scaffold. The carrier 1200 can be a cage. The carrier 1200 can be a tubular structure. The carrier 1200 can be a circumferential structure. The carrier 1200 can be an expandable structure. The carrier 1200 can be a frame. The carrier 1200 can be a mesh. The carrier 1200 can include interwoven fibers. The carrier 1200 can include overlapping struts. The carrier 1200 can include helical struts. The carrier 1200 can have circumferential connections between struts. The carrier 1200 can include elements arranged in a pattern. The carrier 1200 can include a woven network forming numerous small, open spaces. The open spaces can facilitate collapse and expansion in some embodiments. The open spaces can have parallel edges. The carrier 1200 can be braided. The carrier 1200 can be a radially expandable frame. The carrier 1200 can have pairs of struts joined together. The carrier 1200 can include pairs of struts that intersect. The carrier 1200 can be a coil. The carrier 1200 can be a cylindrical frame with struts. The carrier 1200 can be circumferentially connected. The carrier 1200 can be connected along the circumference. The carrier 1200 can have a generally cylindrical shape. The carrier 1200 can receive the balloon 100 therewithin. The carrier 1200 can be bonded to the balloon 100 through any method described herein. The carrier 1200 can be carried by the balloon 100 to the treatment site. The carrier 1200 can surround the balloon 100. The carrier 1200 can be expanded against the vessel by the balloon 100. The carrier 1200 can be pushed against the vessel under the expansion force of the balloon 100.

The carrier 1200 can allow for compression and expansion. The carrier 1200 can be flexible. The carrier 1200 can be able to be navigated through the vessel. The carrier 1200 can be generally cylindrical shaped. The carrier 1200 can be a self-expanding stent. The carrier 1200 can include a lumen to receive the balloon 100. The balloon 100 and the carrier 1200 can be configured for treatment of lesions.

The carrier 1200 can include any raised feature. The carrier 1200 can include any protrusion. The carrier 1200 can include the raised features 300 described herein. The carrier 1200 can include the raised features 300 shown on the strips 200. The carrier 1200 can include the raised features 300 not shown on strips 200. The carrier 1200 can omit the raised features 300. The carrier 1200 can include the raised feature 300 without the strip 200. The balloon 100 and the carrier 1200 can be configured for controlled penetration by the raised features 300. The raised features 300 can be mounted to the carrier 1200 through any means described herein. The raised features 300 can protrude from the carrier 1200. The raised features 300 can be protruding features carried by the carrier 1200. The raised features 300 can be barbs. The raised features 300 can be scoring elements. The raised features 300 can extend radially outward from the carrier 1200. The raised features 300 can extend from the structure of the carrier 1200. The raised features 300 can extend from vertices of the carrier 1200. The raised features 300 can extend from intersecting struts. The raised features 300 can extend perpendicular to the carrier 1200. The raised features 300 can be self-expanding. The raised features 300 can be radially expandable spikes. The raised features 300 can move from a generally tangential orientation to a perpendicular orientation. The raised features 300 can have a shape memory material. The carrier 1200 and the raised features 300 can be integrally formed. The carrier 1200 and the raised features 300 can be separately formed. The carrier 1200 and the raised features 300 can be coupled. The raised features 300 can have any feature described herein. The raised features 300 can be longitudinally arranged. The raised features 300 can be axially aligned. The raised features 300 can be arranged along the same circumferential position. The raised features 300 can function to form serrations as described herein. The raised features 300 can penetrate tissue and/or plaque. In some embodiments, the raised features 300 can be randomly arranged. In some embodiments, the raised features 300 can be arranged in a pattern. In some embodiments, the raised features 300 can be arranged at vertices of the mesh. In some embodiments, the raised features 300 can be helically arranged. The raised features 300 can be arranged with different axial and/or circumferential positions. In some embodiments, the raised features 300 can be along any portion of the carrier 1200.

The raised features 300 can deliver drugs. The raised features 300 can include drug-coated material. The raised features 300 can penetrate tissue to deliver drugs. The raised features 300 can penetrate plaque to deliver drugs. The raised features 300 can deliver drugs upon penetration. The carrier 1200 can be a drug-eluting stent. The carrier 1200 can include drug-coated material. The carrier 1200 can release a drug when the carrier 1200 contacts a vessel wall. The carrier 1200 can directly contact a wall to release a drug. The balloon 100 can be a drug coated balloon. The balloon 100 can directly contact a wall to release a drug.

The carrier 1200 can be any material described herein. The carrier 1200 can be bioresorbable. The carrier 1200 can include a shape memory material. The carrier 1200 can include metal such as nitinol. The carrier 1200 can include a polymer. The carrier 1200 can include one or more radiopaque markers. The carrier 1200 and the raised features 300 can comprise the same material. The carrier 1200 and the raised features 300 can comprise different materials.

The balloon 100 can function as a delivery system for the carrier 1200. The carrier 1200 can be positioned over the balloon 100. The balloon 100 can be without strips 200. The balloon 100 can be without raised features 300. The balloon 100 can include one or more layers described herein. The balloon 100 can be a plain angioplasty balloon described herein. The balloon 100 can be coupled with the generator 800 for pressure wave delivery as described herein. The generator 800 can be used with only the balloon 100. The generator 800 can be used with the balloon 100, wherein the balloon 100 does not include any strips 200 or raised features 300. The balloon 100 can be a dilation balloon. The balloon 100 can stabilize the carrier 1200. The balloon 100 can assist in pushing the raised features 300 into the vessel.

The balloon 100 and the carrier 1200 can be integrated. The balloon 100 and the carrier 1200 can be fixedly coupled. The balloon 100 and the carrier 1200 can deploy and collapse together. The carrier 1200 can be anchored to the balloon 100 such as with the retention balloon 104. The raised features 300 can rise above the retention balloon 104. The balloon 100 and the carrier 1200 can be removable coupled. The balloon 100 and the carrier 1200 can deploy together. The balloon 100 and the carrier 1200 can collapse separately in some methods. The balloon 100 can collapse while the carrier 1200 does not collapse in some methods.

In some embodiments, the balloon 100 can be used with the carrier 1200 according to methods described herein. The carrier 1200 can be separate from the balloon 100. The carrier 1200 can include one or more strips 200. The carrier 1200 can include one or more raised features 300. The strips 200 with the raised features 300 can be indirectly connected to the balloon 100. The balloon 100 itself can produce advantages in combination with the generator 800. The balloon 100 can produce advantages regardless of the structure of the strips 200 and the raised features 300. The balloon 100 and the generator 800 can be used to produce the pressure waves that produce the lithotripsy effects described herein. The balloon 100 and the generator 800 can be used in combination with strips 200 with the raised features 300, whether directly disposed on the balloon 100 or on the carrier 1200. In some embodiments, the raised features 300 can protrude from a surface of the carrier 1200. The balloon 100 can be used in a balloon-assisted technique to expand the carrier 1200. The carrier 1200 can be expanded by the balloon 100 to press the raised features 300 into the surface of the vessel. The raised features 300 can be pressed into the surface of the vessel under the expansion force of the balloon 100 when the balloon 100 is inflated. The raised features 300 can be pressed into the surface of the vessel due to the pressure waves when the balloon 100 is inflated. The balloon 100 can be used as a means to stabilize the carrier 1200. The balloon 100 can be used as a means to press the raised features 300 into the vessel wall. The carrier 1200 can be a means for carrying the raised features 300. In some embodiments, the carrier 1200 can remain within the vessel when the balloon 100 is removed. In some embodiments, the carrier 1200 can be removed with the balloon 100 when the balloon 100 is removed. In some embodiments, the carrier 1200 can be a self-expanding structure. In some embodiments, the carrier 1200 can expand under the influence of the balloon. In some embodiments, the carrier 1200 can be a stent. In some embodiments, the carrier 1200 can be an interwoven structure. In some embodiments, the carrier 1200 can be a mesh. In some embodiments, the carrier 1200 can include one or more strips arranged in a pattern. In some embodiments, the carrier 1200 can be connected circumferentially. The carrier 1200 can have any features of the strips 200 described herein. The carrier 1200 can have a plurality of raised features 300, which can have any feature described herein.

The raised features 300 may be separate from the balloon surface, such as on the carrier 1200 described herein. The raised features 300 of the carrier 1200 can have any shape described herein. The raised features 300 of the carrier 1200 can be blunt. The raised features 300 of the carrier 1200 can be sharpened. The raised features 300 of the carrier 1200 can be a needle point. The raised features 300 of the carrier 1200 can be longitudinally oriented on the carrier 1200. The raised features 300 of the carrier 1200 may be delivered prior to the balloon 100 design to translate pressure wave energy from the balloon surface. The raised features 300 may be part of a design of the carrier 1200 intended to be deployed independent of a balloon. The raised features 300 may be part of a design of the carrier 1200 intended to be deployed separately but in conjunction with the balloon 100. The raised features 300 of the carrier 1200 may be shaped as points of contact. The raised features 300 may not be a wedge shape. The raised features 300 may be configured to raise during deployment.

### Generator

The generator 800 can function to generate pressure waves. The generator 800 can produce a rhythmic pumping action. The generator 800 can provide intermittent pressure changes rather than a continuous pressure. In some embodiments, the generator 800 can be a pulsatile pump. In some embodiments, the generator 800 can mimic the pumping action of the heart. In some embodiments, the generator 800 can replicate a variety of waveforms. In some embodiments, the generator 800 can produce pressure changes. In some embodiments, the generator 800 can produce volumetric displacement. The generator 800 can include the membrane 884, the cam hammer 848, the piston 878, and the spring 846 among other components. The generator 800 can produce pressure waves using these components, as described herein.

The generator 800 can be a pulse driven mechanism. In some embodiments, design of pulse driven mechanisms can include a cam drive behind a flexible membrane or moveable valve. For example, the generator 800 can include the piston 878 driven by the cam hammer 848 behind the membrane 884. The pulse driven mechanisms can create a frequency for the mechanical energy delivery. In some embodiments, the pulse driven mechanism can be separate from the inflation mechanism such as inflation device 400 that inflates and deflates the balloon 100. In some embodiments, the pulse driven mechanism can be integrated into the inflation mechanism that inflates and deflates the balloon 100. In some embodiments, the frequency of the pulses can be tailored to produce the desired lithotripsy effect. In some embodiments, the compliance of the balloon 100 can be tailored to produce the desired lithotripsy effect. In some embodiments, the variation in pressure of the fluid in the balloon 100 can be tailored to produce the desired lithotripsy effect. In some embodiments the variation in pressure carries an ultrasonic wave. In some embodiments, the variation in volume of the fluid in the balloon 100 can be tailored to produce the desired lithotripsy effect. In some embodiments, there can be shift in the radius of the balloon 100 caused the pulse of the pressure wave. The strip 200 can pulse in and out from the pressure wave, for instance between half the height of the strip 200 and the full height of the strip 200. The strip 200 can be used for lithotripsy. The strip 200 can be effective at cracking calcium. The raised features 300 can pulse in and out from the pressure wave, for instance between half the height of the raised features 300 and the full height of the raised features 300. The raised features 300 can be used for lithotripsy. The raised features 300 can be effective at cracking calcium.

The system 1100 can be a serration enhanced and pulse enhanced balloon catheter system. The generator 800 can create pressure waves through the liquid medium used to fill the balloon 100. The generator 800 can create pressure waves through the solid structures of the balloon 100, the strips 200, and the raised features 300. The generator 800 can direct pressure waves from the generator 800 which is proximal to the balloon 100. The generator 800 can provide a proximal source for pressure wave generation. The generator 800 can create ultrasonic pressure waves. The generator 800 can create acoustic pressure waves. The generator 800 can create pressures waves over a low ultrasonic frequency range. The generator 800 can create pressure waves over a low pressure range. The system 1100 can produce a synergistic effect between the serrations and the pressure wave generation.

The generator 800 can include several components that produce the pressure waves. The generator 800 can include the deflectable membrane 884 that produces the pressure wave when deflected. The deflection can be generated by the cam hammer 848 and the piston 878. The deflection can be spring actuated by the spring 846. The generator 800 can function to provide spring actuated movement of the membrane 884. The generator 800 can function to provide percussive movement of the membrane 884. The deflection of the membrane 884 can be caused by a cam-driven actuator. The piston 878 can be driven by the cam hammer 848. The generator 800 can allow the piston 878 to transfer impact energy to the membrane 884, wherein the membrane 884 transfers impact energy to fluid inside the reservoir 854. The generator 800 can allow the piston 878 to displace a defined volume of fluid inside the reservoir 854. The generator 800 can allow energy to be imparted onto fluid inside the reservoir 854. The generator 800 can cause an increase of pressure within the reservoir 854. The generator 800 can cause an increase of pressure within the catheter 600. The generator 800 can cause an increase of pressure within the balloon 100. The generator 800 can move the piston 878 at a speed that generates an ultrasonic pressure wave within the system 1110. The generator 800 can move the piston 878 at a speed that generates an ultrasonic pressure wave that travels through the balloon 100 and into the diseased tissue surrounding the balloon 100. These functions and others will be described herein.

### Generator Subassembly: Internal Support Structure

The generator 800 can include a support structure. The support structure can include a cam bearing block 836, a hammer bearing block 852, and a cylinder 876. The generator 800 can include the cam bearing block 836. The cam bearing block 836 can provide structural support between components of the generator and the housing 802, 806. The cam bearing block 836 can provide structural support for the printed circuit board assembly 816. The printed circuit board assembly 816 can be secured to the cam bearing block 836 using printed circuit board mounting screws 818. The cam bearing block 836 can include a threaded portion 838. The threaded portion 838 can engage an adjuster 822 as described herein. The cam bearing block 836 can be aligned within features of the top housing 802 and the bottom housing 806. The cam bearing block 836 can be prevented from rotating within the top housing 802 and the bottom housing 806. The cam bearing block 836 can be prevented from translating within the top housing 802 and the bottom housing 806. The printed circuit board assembly 816 can be secured to the cam bearing block 836.

The generator 800 can include the hammer bearing block 852. The hammer bearing block 852 can provide structural support between components of the generator and the housing 802, 806. The hammer bearing block 852 can provide structural support for the printed circuit board assembly 816. The hammer bearing block 852 can secure the main printed circuit board 816A using one or more printed circuit board mounting screws 818. The hammer bearing block 852 can set the height of the printed circuit board assembly 816. The hammer bearing block 852 can constrain components of the generator. The hammer bearing block 852 can allow free rotation components of the generator. The hammer bearing block 852 can allow axial translation of the cam hammer 848. The hammer bearing block 852 can constrain rotation of the cam hammer 848. The hammer bearing block 852 can mount to the cam bearing block 836. In some embodiments, the hammer bearing block 852 can be secured to the cam bearing block 836 with one or more screws. The hammer bearing block 852 can fit one or more thrust bearings. The hammer bearing block 852 can fit one or more thrust washer. The hammer bearing block 852 can allow for clearance for the main printed circuit board 816A. The bearing strip 814 can be configured to rotate within a slot of the hammer bearing block 852. The top housing 802 and the hammer bearing block 852 can guide movement of the bearing strip 814. The printed circuit board assembly 816 can be secured to the hammer bearing block 852. The printed circuit board assembly 816 can be secured to the hammer bearing block 852 using printed circuit board mounting screws 818. The cam hammer 848 can translate relative to the hammer bearing block 852. The cam hammer 848 can be prevented from rotating relative to the cam hammer 848.

The generator 800 can include a bearing block screw 896. The bearing block screw 896 can connect the cam bearing block 836 to the hammer bearing block 852.

The generator 800 can include the cylinder 876. The cylinder 876 can thread into the hammer bearing block 852. The cylinder 876 can have a threaded portion and the hammer bearing block 852 can have a threaded portion. The cylinder 876 can engage a bore of the hammer bearing block 852. The cylinder 876 can provide clearance for a sensor port. The cylinder 876 can serve as a mounting surface for the reservoir 854. The cylinder 876 can interface with a feature of the bottom housing 806. In some embodiments, the cylinder 876 can interface with a rib of the bottom housing 806 to prevent rotation when installed. The cylinder 876 can seal a component between the reservoir 854 and the cylinder 876. The hammer bearing block 852 can allow the cylinder 876 to thread into a bore. The hammer bearing block 852 can position the cylinder 876. The reservoir 854 can be secured to the cylinder 876. The reservoir 854 can be secured to the cylinder 876 using one or more screws.

### Generator Subassembly: Cam-Driven Actuator

The generator 800 can include a spline adapter 868. The spline adapter 868 can interface between the motor 830 and a cam can 860. The spline adapter 868 can rotate the cam can 860 as the motor 830 rotates. The spline adapter 868 can translate relative to the cam can 860. The spline adapter 868 can transmit torque to the cam can 860. The spline adapter 868 can rotate freely in a cam bearing block 836. The spline adapter 868 can interface with the motor shaft 831 of the motor 830. The spline adapter 868 can allow thrust washers to be inserted. The spline adapter 868 can allow shims to be inserted. The spline adapter 868 can accept torque and rotation for the motor shaft 831 of the motor 830. The motor 830 can provide torque and rotation to the spline adapter 868. The spring 846 can fit inside a bore of the spline adapter 868. The spline adapter 868 can allow the spring 846 to be inserted. The spring 846 can apply a compressive force between the cam hammer 848 and the spline adapter 868. The cam hammer 848 and the spline adapter 868 can capture the spring 846. The cam can 860 can translate torque from the spline adapter 868 to the cam can insert 862. The cam can 860 can allow axial translation of the spline adapter 868 within the cam can 862.

The generator 800 can include the cam can 860. The cam can 860 can be rotated by the spline adapter 868. The cam can 860 can cause translation of the cam hammer 848. The cam can 860 can convert rotational movement of the motor 830 into translational movement of the cam hammer 848. The cam can 860 can translate torque from the spline adapter 862 to a cam can insert 862. The cam can 860 can be secured to the cam can insert 862. The cam can 860 can provide contrast between white reflective indicators and the body of the cam can 860 to interface with an optical sensor to monitor rotation of the cam can 860. The cam can 860 can allow axial translation of the spline adapter 862 within cam can 860. The cam can 860 can allow translation of the cam hammer 848. The printed circuit board assembly 816 can position a reflective sensor relative to the cam can 860. The printed circuit board assembly 816 can monitor rotation of the cam can 860. The cam can 860 can provide contrast between white reflective indicators and the body of the cam can 860 to interface with Optical IR Sensor on the printed circuit board assembly 816. The hammer bearing block 852 can allow free rotation of the cam can 860.

The generator 800 can include a cam can insert 862. The cam can insert 862 can convert rotational movement of the motor 830 into translational movement of the cam hammer 848. The cam can 860 can secure to the cam can insert 862. The generator 800 can include a keyed interface to allow the cam can insert 862 to align with the cam profile of the cam can 862. In some embodiments, the keyed interface can be both physical and visual. The cam can insert 862 can provide a ramp 864. The ramp 864 can interact with the cam hammer 848. The ramp 864 can include at least one inclined surface. The cam hammer 848 can translate the height of the ramp 864. The cam hammer 848 can translate the difference in height between the lowest point of the ramp 864 and the highest point of the ramp 864. The ramp 864 of the cam can insert 862 can actuate the cam hammer 848. The cam can insert 862 can allow free rotation relative to cam hammer 848. The cam can insert 862 can allow free translation relative to cam hammer 848. The cam can insert 862 can interface with a needle thrust bearing 866 for smooth rotation relative to the hammer bearing block 852. The cam can insert 862 can transmit torque from the cam can 860 to the cam hammer 848. The cam hammer 848 can interface with the cam can insert 862. The cam hammer 848 can interface with the cam can insert 862 to load and unload the spring 846. The cam hammer 848 can interface with the cam can insert 862 to load and unload the spring 846 by translation of the cam hammer 848. The cam can 860 can be secured to the cam can insert 862.

The generator 800 can include a cam hammer 848. The cam hammer 848 can translate as the cam can 860 rotates. The cam hammer 848 can be actuated by the cam can insert 862 of the cam can 860. The cam hammer 848 can strike the piston 878 when actuated. The cam hammer 848 can receive energy from the spring 846. The cam hammer 848 can transfer energy to the piston 878 when striking the piston 878. The cam hammer 848 can capture a portion of the spring 846. The cam hammer 848 load and unload the spring 846. The cam hammer 848 can strike the piston 878 a discrete number of times per complete revolution of the cam can 860. The cam hammer 848 can include a cam surface 850. The cam surface 848 can include one projection, two projections, three projections, four projections, or any range of two of the foregoing values. The cam surface 848 can include one or more projections that serve as an optical reference for a striking position. The striking position can correspond to the striking of the piston 878 with the cam hammer 848. The spring 846 can be at its most extended position in the cycle. The spring 846 can ensure that cam hammer 848 has maximum velocity upon impact. The spring 846 can release its stored energy to drive the cam hammer 848. The striking position can correspond to the maximum induced load on the spring 846. The spring 846 will be at its maximum compression when driving the piston 878. The spring 846 can be compressed to deliver maximum force. The striking position can correspond to the maximum induced load on the spring 846 by the cam hammer 848. The striking position can correspond to a forward position of the cam hammer 848. The striking position can correspond to a compressed position of the spring 846. The striking position can correspond to a forward position of the piston 878. The striking position can correspond to a deflected position of the deflectable membrane 884. The striking position can correspond to a spike of pressure in the balloon 100. The striking position can correspond to the generation of the pressure wave. The cam surface 850 of the cam hammer 848 can include one valley, two valleys, three valleys, four valleys, or any range of two of the foregoing values. The cam surface 848 can include one or more valleys that serve as an optical reference for a home position. The home position can correspond to the base pressure in the system 1100. The home position can correspond to the period between the generation of pressure waves. The home position can correspond to the minimum induced load on the spring 846. The home position can correspond to the minimum induced load on the spring 846 by the cam hammer 848. The home position can correspond to a neutral position of the cam hammer 848. The home position can correspond to a neutral position of the spring 846. The home position can correspond to a neutral position of the piston 878. The home position can correspond to a neutral position of the deflectable membrane 884. The home position can correspond to a low baseline pressure in the balloon 100. The cam surface 850 can include at least one projection and at least one valley. The cam surface 850 can include at least two projections and at least two valleys. The projections can interact with the ramp 864 of the cam can insert 862 to acuate the cam hammer 848 as described herein. The cam hammer 848 can translate. The cam hammer 848 can translate the height of the cam surface 850. The cam hammer 848 can translate the difference in height between a projection and a valley of the cam surface 850. The spring 846 can store compressive energy to apply to the cam hammer 848. The spring 846 can fit inside a cavity 849 of the cam hammer 848. The cavity 849 can be in the head of the cam hammer 848.

The system 1100 can have a dwell time. The dwell time can be the time that the piston 878 is against the membrane 884. The dwell time can be the time that the cam hammer 848 is against the piston 878. The dwell time can be the time that the hammer-piston system of the cam hammer 848 and the piston 878 are against the membrane 884. The dwell time of the hammer-piston system against the membrane 884 at any given intensity is altered by the pulse-to-pulse variation speed (e.g., the 1 Hz, 2 Hz, or 3 Hz controlled by the speed control 810). The dwell time can be affected by one or more of the following factors including the speed of the motor 830, the diameter of the cam can 860, the slope length of the ramp 864 on the cam can insert 862, and/or the arc shape on the ramp 864. The speed of the motor 830 and cam can 860 design together play a role in determining the length of time that the cam hammer 848 dwells against the piston 878 applying a force against the membrane 884. This dwell time of the hammer-piston system 848, 878 against the membrane 884 can directly effect the pressure wave periodicity. The pressure wave periodicity can directly effect the length of time that the balloon 100 can stay at its greatest volume. The high volume of the balloon 100 can increase the outer diameter of the balloon 100. The decrease in the Hertz rate can increase the dwell time of the balloon 100 at the higher diameter. As described herein, the systems and methods can convert mechanical energy into pressure waves. In some embodiments, the dwell time can be a feature of the mechanical system that cannot be achieved with pneumatic impact energy or hydraulic impact energy. The dwell time can be the length of time of consistent, prolonged contact. The dwell time can measure the duration of the interaction between the membrane 884 and other components of the generator 800. The dwell time can be correlated to specific design features of the system, such as the motor 830, the cam can 860, and the cam can insert 862 which can prolong the impact of the hammer-piston system 848, 878 against the membrane 884. The dwell time can be repeatable and consistent. The dwell time can be when the hammer-piston system 848, 878 strikes and stays. The dwell time can be the length of time of the striking position. The dwell time can be the length of time before resetting the system. The dwell time can be the length of time before returning to the home position. The parameters of the dwell time are described herein.

The generator 800 can include the spring 846. The spring 846 can store compressive energy to apply to the cam hammer 848. The spring 846 can fit inside recesses within the cam hammer 848 and the spline adapter 868. The spring 846 can interface with one or more thrust washers 844. The thrust washer 844 can be placed between rotating components such as the cam hammer 848 and stationary components such as the spring 846. The thrust washer 844 can handle axial loads. The spring 846 can be a compression spring. The amplitude of the pressure wave can be determined by the compression of the spring 846, which can be adjusted as described herein. The compression of the spring 846 can be within a range determined by the structure of the generator 800, such as stops that limit translation of the adjuster 822 as described herein. The spring 846 can only be compressed within a range, thereby limiting the upper limit of the pressure described herein.

The generator 800 can include the piston 878. The piston 878 can transfer impact energy from the cam hammer 848. The piston 878 can transfer energy through the membrane 884 and into fluid of the reservoir 854. The piston 878 can reduce impact sound produced by striking the piston 878. The piston 878 can include materials to dampen sound. The piston 878 can include a material along a core of the piston. The piston 878 can include sealing components. The piston 878 can freely translate within the cylinder 876. The piston 878 can translate forward and backwards. The piston 878 can have travel limited by the cylinder 876. The cylinder 876 can include an edge to limit movement of the piston 878. The piston 878 can interface with or abut an edge of the cylinder 876 to prevent the piston from being retracted too far. The cam hammer 848 can receive energy from spring 846. The cam hammer 848 can transfer energy to the piston 878. The reservoir 854 can allow free travel of the piston 878. The cylinder 876 can allow for smooth axial translation of the piston 878.

The generator 800 can include a piston o-ring 880. The piston o-ring 880 can provide backup sealing. The piston o-ring 880 can seal an interface with the piston 878 and the cylinder 876.

### Generator Subassembly: Reservoir and Membrane

The generator 800 can include the reservoir 854. The reservoir 854 can hold fluids. The reservoir 854 can be in fluid communication with the catheter 600 and the balloon 100. The membrane 884 can be a movable wall of the reservoir 854. The membrane 884 can be deflected to cause a pressure wave within the fluid of the reservoir 854. The pressure wave 854 can originate in the reservoir 854 before propagating through the catheter 600 to the balloon 100. The shape of the reservoir 854 can focus the pressure wave. The reservoir 854 can be funnel shaped. The shape of the reservoir 854 can accelerate the pressure wave as the pressure wave exits the reservoir 854. The reservoir can be located within the proximal handle of the generator 800. The reservoir 854 can secure to the cylinder 876 described herein using one or more screws. The reservoir 854 can provide a sealing surface. The reservoir 854 can provide a back-up o-ring seal. The reservoir 854 can have a connector 856. The connector 856 can allow the balloon 100 to be inflated by the inflation device 400. The connector 856 can be a standard Luer thread connector. The connector 856 can include a bleed port. The reservoir 854 can include one or more sensors to monitor a parameter of the reservoir 854. The reservoir 854 can be transparent. The reservoir 854 can maintain clarity for user to observe air bubbles. The generator 800 can include one or more ports or pathways to remove bubbles. The generator 800 can allow for a method of bubble removal. The reservoir 854 can allow free travel of components. The reservoir 854 can direct air bubbles toward the bleed port. The reservoir 854 can aid in bleeding procedure. The reservoir 854 can prevent air in the system. The hammer bearing block 852 can bleed the Luer port to the top housing 502. The generator 800 can include adhesive to secure one or more components. The adhesive can be Loctite™. The adhesive can created a seal between the reservoir 854 and another component, such as a sensor.

The generator 800 can include a reservoir radial o-ring 882. The reservoir radial o-ring 882 can provide backup sealing. The reservoir radial o-ring 882 can seal an interface with the cylinder 876 and the reservoir 854.

The generator 800 can include the membrane 884. The membrane 884 can be thin. The membrane 884 can be flexible. The membrane 884 can be pliable. The membrane 884 can be a diaphragm. The membrane 884 can be a partition. The membrane 884 can be a sheet. The membrane 884 can be act as a boundary for the reservoir 854. The membrane 884 can separate the reservoir 854 from the piston 878. The membrane 884 can be a batter head. The membrane 884 can be a drum batter. The membrane 884 can be a drum head which is struck to produce sound. The membrane 884 can be a surface that is physically struck by the piston 878. The membrane 884 can create the pressure wave when struck. The membrane 884 can create sound when struck. The membrane 884 can create the acoustic wave when struck. The membrane 884 can create the fluid wave when struck. The membrane 884 can seal fluid inside the reservoir 854. The membrane 884 can seal fluid between the cylinder 876 and the reservoir 854. The membrane 884 can allow the piston 878 to transfer impact energy to fluid inside the reservoir 854. The membrane 884 can be deflected by the piston 878. The membrane 884 can allow the piston 878 to transfer impact energy by deflecting. The membrane 884 can be configured to be deflected. The membrane 884 can be able to elongate to allow maximum extension of the piston 878. The piston head 887 of the piston 878 can be concave. The piston head 887 of the piston 878 can be convex. The piston head 887 of the piston 878 can be flat. The piston head 887 of the piston 878 can contact the membrane 884. The piston head 887 of the piston 878 can deflect the membrane 884 to cause the pressure wave. The reservoir 854 can provide a sealing surface for the membrane 884. The cylinder 876 can seal membrane between the reservoir 854 and the cylinder 876. The piston 878 can transfer impact energy from cam hammer 848 to membrane 884 by deflecting membrane 884. The membrane 884 can be considered a movable wall. The membrane 884 can be made of a highly durable and flexible material, such as silicone. The membrane 884 can be concave. The membrane 884 can be convex. The membrane 884 can be flat. The generator 800 can include a membrane-diaphragm acoustic signal intravascular lithotripsy system.

The generator 800 can include a pressure sensor 888. The pressure sensor 888 can monitor pressure within the reservoir 854. The pressure sensor 888 can monitor pressure during generation of pressure waves. The pressure sensor 888 can accept an upper limit and a lower limit of device pressures. The pressure sensor 888 can provide a signal to the printed circuit board assembly 816 if the reservoir pressure drops below a set point. The pressure sensor 888 can provide a signal to the printed circuit board assembly 816 if the reservoir pressure is above a set point. The pressure sensor 888 can be installed in the reservoir 854. The printed circuit board assembly 816 can allow the pressure sensor 888 to be installed in the reservoir 854. The hammer bearing block 852 can align the pressure sensor 888. The reservoir 854 can secure to the pressure sensor 888. The reservoir 854 can secure to the pressure sensor 888 using adhesive. The adhesive can create a seal between the reservoir 854 and the pressure sensor 888. The pressure sensor 888 can monitor the low end of the pressure. The pressure sensor 888 can monitor the base pressure. The pressure sensor 888 can monitor the back pressure of the system. The spring 846 can limit the high end of the pressure. The spring 846 can only provide so much momentum. In some embodiments, the pressure cannot exceed the threshold due to the compression limits of the spring 846.

In some embodiments, the system 1100 can be sensitive to too little pressure. The base pressure can be used to reset the membrane 884 to the home position. The base pressure can be used to reset the piston 878 to the home position. The base pressure can be used to reset the spring 846 to the home position. The base pressure can be used to reset the system to allow the pressure waves to be generated. The home position can correspond to the minimum induced load on the spring 846. The home position can correspond to a neutral position of the cam hammer 848. The home position can correspond to a neutral position of the spring 846. The home position can correspond to a neutral position of the piston 878. The home position can correspond to a neutral position of the deflectable membrane 884. The home position can correspond to the fluid in the system being at the base pressure. The system 1100 can return to the base pressure after each pressure wave as further described herein. The pressure sensor 888 can confirm there is enough pressure in the system 100 to return to the home position. The pressure sensor 888 can confirm there is enough pressure in the system 100 to reset after each strike. The pressure sensor 888 can ensure the base pressure is 0.5 ATM, 1 ATM, 1.5 ATM, 2 ATM, 2.5 ATM, 3 ATM, at least 0.5 ATM, at least 1 ATM, or any range of two of the foregoing values. The pressure sensor 888 can provide insight into what is happening at the balloon 100. The pressure sensor 888 can ensure that enough pressure is provided to enable the balloon 100 to contact the vessel wall. The pressure sensor 888 can ensure that enough pressure is provided to enable the balloon 100 to apply a force on the vessel wall. The pressure sensor 888 can provide feedback related to the base pressure.

The pressure sensor 888 can detect a drop in pressure in the system 1100. The drop in pressure can correspond to the crack formation. The drop in pressure can correspond to segmentation of the plaque. The drop in pressure can correspond to compression of the plaque. The drop in pressure can correspond to lithotripsy. The drop in pressure can correspond to shearing of the calcium layer. The drop in pressure can correspond to lumen gain. The pressure sensor 888 can provide insight into what is happening at the vessel. The pressure sensor 888 can provide feedback the vessel has yielded.

The pressure sensor 888 can be used in a feedback loop design. The pressure sensor 888 can sense the pressure of the system 1100 and produce a data stream. The software and/or processor in the system 1100 can collect this data stream and perform a variety of functions based on the data points collected by the pressure sensor 888. In some designs, the software and/or processor can turn off power to the motor 830 when the pressure falls below a limit point of pressure. The limit point of pressure for the software to take an action can be preset in the software. The limit point of pressure to control the motor 830 can be set by the operator. The limit point of pressure to control the motor 830 can be driven by an artificial intelligent program. The computer program can be configured to monitor the sensor input and perform tasks such as reasoning, learning, problem-solving, decision-making to produce a desired outcome such as more effective stenosis reduction.

The generator 800 can include a pressure sensor retainer 886. The pressure sensor retainer 886 can interface with the hammer bearing block 852. The pressure sensor retainer 886 can fasten to the hammer bearing block 852 with one or more screws. The pressure sensor retainer 886 can interface with a rigid section of the main printed circuit board 816A. The pressure sensor retainer 886 can interface with the printed circuit board assembly 816 where the pressure sensor 888 is mounted. The pressure sensor retainer 886 can provide a retention force to hold the pressure sensor 888 in place during operation and shelf life. The hammer bearing block 852 can constrain the pressure sensor retainer 886. The hammer bearing block 852 can align the pressure sensor retainer 886. The cylinder 876 can provide clearance for the pressure sensor retainer 886.

The generator 800 can include a pigtail tube 890. The pigtail tube 890 can be coupled to the reservoir 854. In some embodiments, the pigtail tube 890 can be solvent bonded to the reservoir 854. The reservoir 854 can secure to the pigtail tube 890. The pigtail tube 890 can be connected to a catheter 600 that is connected to the proximal end of the balloon 100. The reservoir 854 can be configured to be filled with an incompressible fluid. The pigtail tube 890 can be configured to be filled with an incompressible fluid. The catheter 600 can be configured to be filled with an incompressible fluid. The balloon 100 can be configured to be filled with an incompressible fluid. The reservoir 854 can be fluidically connected to the balloon 100.

In FIGS. 6-7, the system 1100 can include the generator 800 which can be external to the body of the balloon 100. The balloon 100 can be connected to the catheter 600. The reservoir 854 can secure to the pigtail tube 890 and the pigtail tube 890 can be connected to the catheter 600. The catheter 600 can be connected to the proximal end of the balloon 100. The balloon 100 can be in fluidic communication with the reservoir 854 via the catheter 600. The generator 800 can be connected proximal to the proximal cone of the balloon 100. The generator 800 can be connected proximal to the proximal leg of the balloon 100. The generator 800 can be connected proximal to the catheter 600.

The generator 800 can generate the pressure wave. The generator 800 can be the pressure wave source. The generator 800 can be connected proximal to the proximal cone of the balloon 100. The generator 800 can be connected proximal to the proximal leg of the balloon 100. The generator 800 can be external to the balloon 100. The generator 800 can be external to the body of the patient. The generator 800 can be extracorporeal. In some embodiments, the pressure wave can be generated from a line source outside the body of the balloon 100. In some embodiments, the pressure wave can be generated from sources proximal to the body of the balloon 100. In some embodiments, the pressure wave can be generated from sources extracorporeally. In some generators that sit extracorporeally, the pressure wave can enter the balloon 100 through the catheter 600 connected to the proximal balloon leg of the balloon 100. The catheter 600 can include a main shaft and an inner member. In some embodiments, fluid medium fills the space between the main shaft and the inner member of the catheter 600. In some embodiments, when the pressure wave is transmitted through the catheter 600, the pressure wave can travel through the cavity in the space between the main shaft and the inner member of the catheter 600. In some embodiments, the generator 800 can have a chamber shape of the reservoir 854 that directs the pressure wave down the catheter 600 minimizing acoustic energy loss through the main shaft and into the vessel proximal of the balloon 100. The reservoir 854 can direct the pressure wave toward the balloon 100. The pressure wave can be delivered along the length of the balloon 100. The pressure wave can be delivered distal to the length of the balloon 100. The design of the spacing between the main shaft and the inner member of the catheter 600 can be made to promote this translation of the pressure wave. In some embodiments, the length of the catheter 600 can be shorter than the attenuation of the frequency being transmitted. In some embodiments, the walls of the catheter 600 can be configured to limit acoustic dampening.

### Generator Subassembly: Adjuster

The generator 800 can include an adjuster 822. The adjuster 822 can be rotated by the user. The adjuster 822 can be rotated by the user to change the compression of the spring 846. The adjuster 822 can be rotated by the user to change the force of impact of the piston 878. The adjuster 822 can be rotated by the user to change the amplitude of the pressure wave. The adjuster 822 can be rotated by the user to increase or decrease the spring load to in turn adjust the characteristics of the pressure wave produced. The adjuster 822 can increase the pressure of the system 1100. The adjuster 822 can decrease the pressure of the system 1100.

The adjuster 822 can include grips to facilitate rotation. The adjuster 822 can include longitudinal ridges around the circumference of the adjuster 822. In some embodiments, the adjuster 822 can move freely within the top housing 802 and the bottom housing 806. In some embodiments, the adjuster 822 can rotate freely relative to the top housing 802 and the bottom housing 806. In some embodiments, the top housing 802 can have features to limit movement of the adjuster 822. The top housing 802 can constrain the inner assembly from rotating. In some embodiments, the bottom housing 806 can have features to limit movement of the adjuster 822. The bottom housing 806 can constrain the inner assembly from rotating. The adjuster 822 can rotate freely around a cable for the printed circuit board assembly 816. The adjuster 822 can rotate freely around a cable for the main printed circuit board 816A

The adjuster 822 can be include a threaded barrel. The adjuster 822 can comprise a threaded portion 824. The adjuster 822 can thread into the cam bearing block 836. The threaded portion 824 of the adjuster 822 can engage the threaded portion 838 of the cam bearing block 836 . . . . The adjuster 822 can prevent gross water ingress by interfacing with a cable strain relief 832, as described herein. The adjuster 822 can prevent user pinch between the top housing 802 and the bottom housing 806. The adjuster 822 can provide user interface for adequate grip to actuate. The adjuster 822 can isolate the user from excessive temperatures of the motor 830. The adjuster 822 can maintain integrity during use.

The adjuster 822 can be moved by the user. The adjuster 822 can rotate. The adjuster 822 can translate as the adjuster 822 rotates. The adjuster 822 can move freely within the top housing 802 and the bottom housing 806. The top housing 802 and the bottom housing 806 can remain stationary as the adjuster 822 rotates and translates. The adjuster 822 can thread into the cam bearing block 836. The adjuster 822 can rotate and translate relative to the cam bearing block 836. The cam bearing block 836 can remain stationary as the adjuster 822 rotates and translates. The cam bearing block 836 can be fixed relative to the top housing 802 and the bottom housing 806.

The adjuster 822 can rotate and translate to change the position of internal components. The adjuster 822 can rotate and translate to change the position of the motor 830. The motor 830 can have limited translation within the top housing 802 and the bottom housing 806. The motor 830 can be prevented from rotating within the top housing 802 and the bottom housing 806. The motor 830 can only translate. The motor 830 can only translate as the adjuster 822 rotates and translates. Several components translate as the motor 830 translates. In some embodiments, the proximal printed circuit board cage 834 and the cable strain relief 832 translate with the motor 830. In some embodiments, the motor printed circuit board 816B translates with the motor 830. In some embodiments, a cable assembly 826 translates with the motor 830. In some embodiments, the motor mount pre-load block 840 translates with the motor 830. In some embodiments, the spline adapter 868 translates with the motor 830. The spring 846 can be received within the spline adapter 868. The spring 846 can be received between the spline adapter 868 and the cam hammer 848. The spring 846 can be compressed between the spline adapter 868 and the cam hammer 848 as the motor 830 translates. The spring 846 can be compressed when the adjuster 822 is translated distally. The spring 846 can be decompressed when the adjuster 822 is translated proximally. The adjuster 822 can move the motor 830 which can cause compression of the spring 846. The amplitude of the pressure wave can be adjusted by translation of the adjuster 822. The amplitude of the pressure wave can be adjusted by translation of the motor 830. The amplitude of the pressure wave can be adjusted by compression of the spring 846.

In some embodiments, the generator 800 is configured to limit the amplitude of the pressure wave by creating stops for movement of the adjuster 822. The adjuster 822 can have limits to movement. The adjuster 822 can abut a stop within the handle at the distal end of travel. The adjuster 822 can abut a stop within the handle at the proximal end of travel. The adjuster 822 can have limited travel to limit over compression of the spring 846. The adjuster 822 can have limited travel to maintain the amplitude of the pressure wave below a threshold.

The generator 800 can include a position sensor system to determine the position of the adjuster 822. The position sensor system can be a magnetic position sensor system. The generator 800 can include a magnet 858. The magnet 858 can provide feedback for the adjuster 822. The magnet 858 can secure to the motor mount pre-load block 840. In some embodiments, the magnet 858 can be secured to the motor mount pre-load block 840 by a heat stake. In some embodiments, the magnet 812 can be secured to the motor mount pre-load block 840 by a fastener such as screws or adhesive. The magnet 858 can provide feedback on position of the adjuster 822. The magnet 858 can provide consistent magnetic field for interaction with a detector of the position sensor system. The position sensor system can utilize other types of sensors including potentiometric sensors, optical encoders, inductive sensors, among other sensor types. The printed circuit board assembly 816 can detect the magnet 858 to determine the position of the adjuster 822. The printed circuit board assembly 816 can read the position of the adjuster 822 via the magnet 858 installed in the motor mount pre-load block 840. The motor mount pre-load block 840 can include the magnet 858. The printed circuit board assembly 816 can determine the location of the magnet 858. The location of the magnet 858 can indicate the position of the adjuster 822. The location of the magnet 858 can indicate the position of the motor 830. The location of the magnet 858 can indicate the compression of the spring 846. The location of the magnet 858 can indicate the amplitude of the pressure wave based on the compression of the spring 846.

The user can select the force of the pressure wave by moving the adjuster 822. The user can rotate adjuster 822 to increase the maximum pressure of the pressure wave delivered by the generator 800. The user can rotate adjuster 822 to increase the compression of the spring 846. The user can rotate adjuster 822 to decrease the maximum pressure of the pressure wave delivered by the generator 800. The user can rotate adjuster 822 to decrease the compression of the spring 846. The position of the magnet 858 of the motor mount pre-load block 840 can be detected by the printed circuit board assembly 816. The printed circuit board assembly 816 can provide an indication of pressure depending on the position of the magnet 858 of the motor mount pre-load block 840. The system 1100 can include one or more LED lights. The LED lights can be illuminated based on the position of magnet 858. The LED lights can be illuminated based on the position of the motor mount pre-load block 840. The motor mount pre-load block 840 with the magnet 858 can be translated by the adjuster 822. The LED lights can be illuminated based on position of the magnet 858. The LED lights can be illuminated based on the compression of the spring 846. The LED lights can be illuminated and viewable through the top housing 802.

### Generator Subassembly: Motor

The generator 800 can include the motor 830. In some embodiments, the motor 830 can be miniature motor, such as a 28 mm motor. The generator 800 can include wire leads to secure the motor 830 to the motor printed circuit board 816B. The motor 830 can secure to other components such as the proximal PCB cage 834 with snaps. The motor 830 can secure to other components such as the motor mount pre-load block 840 with screws. The motor 830 can provide torque and rotation to other components such as the spline adapter 868. The motor 830 can provide torque and rotation to the cam can 860 and cam can insert 862. The rotation of the motor 830 can cause cam hammer 848 to translate. The rotation of the motor 830 can cause cam hammer 848 to strike the piston 878. The rotation of the motor 830 can cause piston 878 to strike the membrane 884. The rotation of the motor 830 can membrane 884 to be deflected. The rotation of the motor 830 can cause the generation of the pressure wave. The generator 800 can be motor driven.

The motor 830 can be translated proximally and distally by the adjuster 822. The motor 830 can allow clearance for the adjuster 822. The motor 830 can allow translation and rotation of the adjuster 822. The adjuster 822 can rotate freely around the motor 830. The motor 830 can provide cable strain relief when the adjuster 822 is rotated. The motor 830 can prevent the cable assembly 826 from spinning when the adjuster 822 is rotated. The motor 830 can provide sufficient torque and rotation to a drive system. The motor 830 can include a motor controller for interfacing with system control. The motor 830 can output speed feedback. The motor 830 can accept speed control input. The speed of the motor can be controlled by speed control 810. The speed control 810 can adjust the speed of the motor 830, such as 1 Hz, 2 Hz, 3 Hz. The motor 830 can be compatible with 20-30 VDC power. The motor 830 can receive directional control input. The motor 830 can maintain integrity during use. The printed circuit board assembly 816 can provide detachable connection to the motor 830. The adjuster 822 can isolate the user from excessive temperatures of the motor 830. The motor 830 can include a motor shaft 831. The motor shaft 831 can rotate. The printed circuit board assembly 816 can provide a detachable connection to the motor 830. The adjuster 822 can freely rotate around the motor 830. The motor 830 can be a non-stepper motor. The motor 830 can be a continuous rotation motors. The motor 830 can rotate smoothly.

The generator 800 can include the motor mount pre-load block 840. The motor mount pre-load block 840 can move with the motor 830 as the motor moves. The motor mount pre-load block 840 can secure to the motor 830 with one or more screws. The motor mount pre-load block 840 can prevent rotation of the motor mount pre-load block 840 and the motor 830 relative to the cam bearing block 836. The motor mount pre-load block 840 can be prevented from rotating relative to other components. The motor mount pre-load block 840 can allow axial translation relative to the cam bearing block 836. The motor mount pre-load block 840 can slide relative to other components. The motor mount pre-load block 840 can prevent over pressurization of the system by limiting translating of the motor 830. The motor mount pre-load block 840 can prevent over pressurization by abutting the cam bearing block 836. The motor mount pre-load block 840 can prevent over pressurization of the system by limiting compression of the spring 846. The motor mount pre-load block 840 can allow free rotation of the motor shaft 831. The motor shaft 831 can rotate within a bore of the motor mount pre-load block 840. The motor mount pre-load block 840 can fit one or more thrust bearings. The motor mount pre-load block 840 can fit one or more thrust washer. The motor mount pre-load block 840 can allow the main printed circuit board 816A to pass through.

The spring 846 can provide a force to maintain interface between the motor mount pre-load block 840 and the adjuster 822. The spring 846 can provide a force on the motor mount pre-load block 840. The spring 846 can provide a force to move the motor mount pre-load block 840 proximally. The adjuster 822 can compress the spring 846 by moving the motor mount pre-load block 840 distally.

The motor 830 can be a synchronous motor. The synchronous motor 830 can produce constant speed regardless of load compared to non-synchronous motor. The synchronous motor can operate without slip. The synchronous motor 830 can be superior for precise, high-torque, low-speed applications such as the generator 800. The motor 830 can produce smooth motion compared to a non-synchronous motor. The motor 830 can operate at a precise speed compared to a non-synchronous motor. The motor 830 can have a constant RPM compared to a non-synchronous motor. The motor 830 can provide consistent, periodic motion to the fluid of the system 1100. The motor 830 can result in steady-state pressure wave generation compared to a non-synchronous motor. The motor 830 can produce the fluid wave of the pressure wave. The motor 830 can create a predictable, consistent fluid wave. The motor 830 can ensure that pulses of the pressure wave occur at precise frequencies. The motor 830 can produce a steady, recurring pressure wave. The motor 830 can ensure that the wave frequency remains exactly the same over time. The motor 830 can run at very low speeds, such as 1 Hz, 2 Hz, and 3 Hz. This allows for the slow-motion movements needed to create specific fluid waves in the balloon 100. The motor 830 may not be a stepper motor. Stepper motors can produce jerky, stepped motion. Stepper motors can move in discrete increments or steps. Stepper motors may be unable to produce the fluid wave described herein.

The motor 830 can have a variety of features. The motor 830 can be a brushless direct current electric motor, also known as an electronically commutated motor. The motor 830 can be a synchronous motor using a direct current electric power supply. The motor 830 can move at very low frequencies, thereby produce a slow, precise and constant speed for the generation of pressure waves. The motor 830 can maintain a constant speed for actuating the cam hammer 848. The cam hammer 848 periodically impacts the piston 878. The frequency of impact of the cam hammer 848 can be determined in part by the rotation of the motor shaft 831 which rotates the cam can 860 and the cam can insert 862. The cam can insert 862 that interacts with the cam hammer 848 to translate the cam hammer 848 as the cam can insert 862 rotates. The compression of the spring 846 can change the amplitude of the force applied by the cam hammer 848. The impact force of the cam hammer 848 can be increased or decreased by the force of the spring 846. The spring 846 can be compressed and released as the cam hammer 848 translates. The spring 846 can be loaded and unloaded as the cam hammer 848 translates. The spring 846 can store energy when compressed and then release that energy to propel the cam hammer 848 forward. The spring 846 can store energy to increase impact force. The spring 846 can facilitate rebound the cam hammer 848. The spring 846 can provide consistent, controlled resistance within the generator. The frequency of impact of the piston 878 can be determined in part by the rotation of the motor 830. The frequency of impact of the membrane 884 can be determined in part by the rotation of the motor 830. The striking of the membrane 884 generates the pressure wave. The pressure wave propagates from the reservoir 854 to the liquid medium of the balloon 100 which transfers the wave to the tissue to be treated.

The rotations of the cam can 860 and the cam can insert 862 can be driven by the motor 830. The motor 830 can be driven by a power supply. The motor shaft 831 can be rotated at a rotational speed to create periodic pressure waves. The speed of the motor shaft 831 can be adjusted to adjust the relative impact frequency of the cam hammer 848. The motor 830 can be driven at speeds under 5 Hz (300 rpm). The motor 830 can be driven at 1 Hz. The motor 830 can be driven at 2 Hz. The motor 830 can be driven at 3 Hz. The user can select the speed of the motor 830 by the speed control 810. The position of the magnet 812 of the speed control 810 can be determined by the system 1100. The printed circuit board assembly 816 can adjust the speed of the motor 830 depending on the position of the magnet 812 of the speed control 810.

The motor 830 can drive the cam-based generator. The motor 830 can translate the cam hammer 848. The cam hammer 848 engages periodically with the ramp 864 of the cam can insert 862. The cam hammer moves proximally or distally by the height of the ramp 864, thereby compressing the spring 846 by this height. The impact energy can be stored by the spring 846 can be released nearly instantaneously as the cam hammer 848 drops off the ramp 864 of the cam can insert 862. The spring force can be generated by the unloading of the spring 846. The spring force can be generated by the release of compression in the spring 846. The piston 878 can be acted on nearly instantaneously by the cam hammer 848. The membrane 884 can be acted on nearly instantaneously by the piston 878. The impact can produce pressure waves which propagate through the liquid medium to the vessel to be treated. The resulting pressure waves can break down calcifications in the vessel tissue and calcifications adhered to the vessel wall. The pressure wave can be driven by pressure fluctuations within the balloon 100.

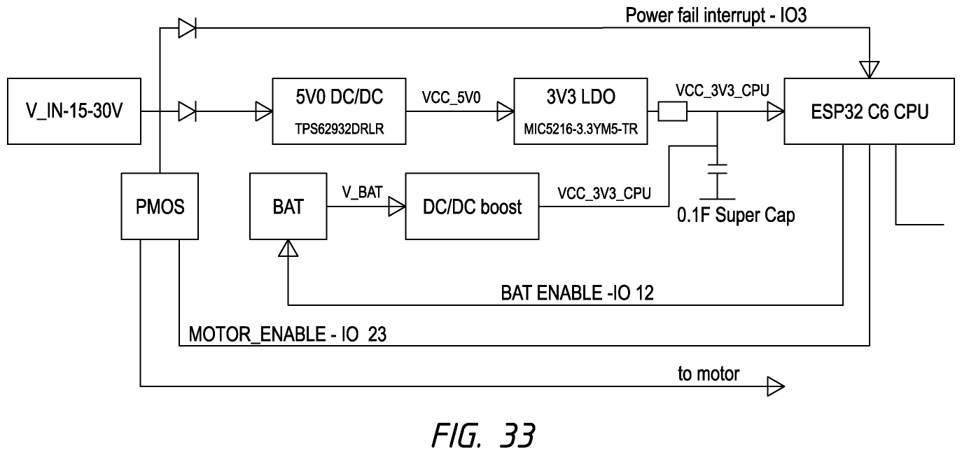

FIG. 33 illustrates an electrical diagram. The operating voltage can be provided by the power supply. The power supply can provide a constant, unidirectional flow of electricity. The operating voltage can maintain a stable polarity. The operating voltage, such as 30 VDC, can go to the motor 830 through a fuse and on/off switching. The operating voltage, such as 30 VDC, can be stepped down to 5V and 3.3V for the printed circuit board assembly 816. The printed circuit board assembly 816 can include button and/or lights as described herein. The operating voltage can be stepped down for components other than the motor 830.

### Generator Subassembly: Power Supply

The generator 800 can include the cable assembly 826. The cable assembly 826 can connect to a power supply. The cable assembly 826 can transmit power from the power supply to the printed circuit board assembly 816. The cable assembly 826 can transmit power from the power supply to the motor printed circuit board 816B. The cable assembly 826 can be connected to the motor printed circuit board 816B. The cable assembly 826 can prevent a user from exposure to electricity. The cable assembly 826 can maintain integrity during use. The cable assembly 826 can include a connector 828. The connector 828 can connect to the power supply. The cable assembly 826 can include a single cable or multiple cables.

The generator 800 can include a cable strain relief 832. The cable strain relief 832 can be connected to the cable assembly 826. The cable strain relief 832 can minimize the bending strain of the cable assembly 826. The cable strain relief 832 can prevent gross water ingress by interfacing with the adjuster 822. The cable strain relief 832 can interface with components to prevent the cable assembly 826 from becoming disconnected from the motor printed circuit board 816B. The cable strain relief 832 can maintain integrity during use. The adjuster 822 can rotate freely around the cable strain relief 832. The cable assembly 826 can pass through the cable strain relief 832.

The cable assembly 826 can form the proximal end of the system 1100. The cable assembly 826 can be connected to the power supply via the connector 828. The cable assembly 826 can bend and twist along the length of the cable assembly 826. The cable assembly 826 can be connected to cable strain relief 832. The cable strain relief 832 can be connected to the adjuster 822. The adjuster 822 can rotate freely around the cable strain relief 832. In some embodiments, the cable strain relief 832 and the adjuster 822 create a seal to prevent fluid ingress. The cable assembly 826 can extend into the proximal end of the adjuster 822. The cable assembly 826 can be coupled to the cable clip 892. The cable clip screw 894 and the cable clip 892 can function as a clamp. The cable clip screw 894 and the cable clip 892 can clamp a portion of the cable assembly 826. The cable strain relief 832 and the proximal printed circuit board cage 834 can be coupled. The proximal printed circuit board cage 834 can be coupled to the motor printed circuit board 816B. The motor printed circuit board 816B can be connected to the motor 830. The proximal printed circuit board cage 834 can be connected to the motor 830. The adjuster 822 can rotate freely around the motor 830.

The generator 800 can include a cable clip 892. The cable clip 892 can secure to the cable assembly 826. In some embodiments, the cable clip 892 can secure to the cable assembly 826 with a cable clip screw 894. The cable clip 892 can interface with the cable strain relief 832 to prevent the cable assembly 826 from being pulled out of the connector. The cable clip 892 can prevent the cable assembly 826 from rotating relative to the cable strain relief 832. The cable assembly 826 can secure to the cable clip 892 with the cable clip screw 894. The cable strain relief 832 can interface with the cable clip 892 to prevent the cable assembly 826 from becoming disconnected from the motor printed circuit board 816B.

The device can have proximal components that facilitate the delivery of power. The cable assembly 826 can connect to the power supply. The cable assembly 826 can supply power to the printed circuit board assembly 816. The cable assembly 826 can pass through the cable strain relief 832. The cable strain relief 832 can minimize bending strain of the cable assembly 826. The cable clip screw 894 and the cable clip 892 can couple to the cable assembly 826. The cable strain relief 832 and the proximal printed circuit board cage 834 can be coupled. The motor printed circuit board 816B can be coupled to the proximal PCB cage 834. The proximal printed circuit board cage 834 can coupled to the motor 830.

Generator Subassembly: Additional features

The generator 800 can include features to reduce friction between components. The generator 800 can include features to handle loads. The generator 800 can include spring shams 898. The spring shams 898 can be inserted into the spline adapter 868. The spring shams 898 can adjust the spring free length relative to the received length. The spline adapter 868 can allow the spring shams 898 to be inserted.

The generator 800 can include a spring thrust washer 870. The spring thrust washer 870 can prevent wear and/or galling of the spline adapter 868. The spring thrust washer 870 can fit within the spline adapter 868. The spring 846 can interface with the spring thrust washer 870. The spline adapter 868 can allow the spring thrust washer 870 to be inserted.

The generator 800 can include a motor thrust bearing 872. The motor thrust bearing 872 can fit in the motor mount pre-load block 840. The motor thrust bearing 872 can interface with the spline adapter 868. The motor thrust bearing 872 can allow for smooth rotation between components. The motor thrust bearing 872 can withstand a thrust load. In some embodiments, the motor mount pre-load block 840 can fit the motor thrust bearing 872.

The generator 800 can include a needle thrust bearing 874. The needle thrust bearing 874 can fit in the hammer bearing block 852. The needle thrust bearing 874 can interface with the cam can insert 862. The needle thrust bearing 874 can allow for smooth rotation between components. The needle thrust bearing 874 can withstand a thrust load. In some embodiments, the hammer bearing block 852 can fit the needle thrust bearing 874.

### Function of the Generator

The generator 800 can be held by the user. The generator 800 can be the proximal handle of the system 1100. The generator 800 can be a compact assembly that can be intuitive for the user to use. The generator 800 can allow the user to adjust parameters of the pressure waves, as described herein. The generator 800 can allow the user to adjust the speed of the motor 830. The generator 800 can allow the user to adjust the compression of the spring 846. The generator 800 can allow the user to turn on or off the generation of pressure waves. The generator 800 can include various feedback controls to monitor parameters as described herein.

The generator 800 can include the top housing 802 and the bottom housing 806. The top housing 802 and the bottom housing 806 can form the majority of the proximal handle. The top housing 802 and the bottom housing 806 can allow the user to easily hold the generator 800. In some embodiments, there can be several components that remain stationary in use. In some embodiments, there can be several components that are fixed to each other in use. In some embodiments, there can be several components that do not rotate or translate in use. The top housing 802 can be fixed. The bottom housing 806 can be fixed. The top housing 802 and the bottom housing 806 can be fixed as the user holds the device. The top housing 802 and the bottom housing 806 can form a fixed reference frame for other movable components of the generator 800. Other components can translate relative to the top housing 802 and the bottom housing 806 such as the motor 830. Other components can rotate relative to the top housing 802 and the bottom housing 806 such as the adjuster 822. While the top housing 802 and the bottom housing 806 are illustrated as separate components, the top housing 802 and the bottom housing 806 can be combined into one housing. The top housing 802 and the bottom housing 806 can include features to facilitate movement of other components. The top housing 802 and the bottom housing 806 can include slots or grooves that facilitate limited movement of internal components such as the bearing strip 814. The top housing 802 and the bottom housing 806 can include projections or stops that facilitate limited movement of internal components such as the bearing strip 814.

The motor 830 can be coupled to multiple components such as the motor mount pre-load block 840 and the spline adapter 868. These components translate as the motor 830 translates. The adjuster 822 can abut the motor mount pre-load block 840, wherein translation of the adjuster 822 can cause corresponding translation of the motor mount pre-load block 840. The motor mount pre-load block 840 can provide a bearing surface between the motor 830 and the adjuster 822. The motor mount pre-load block 840 can be secured to the motor 830. The motor 830 moves with the motor mount pre-load block 840. The adjuster 822 can translate the motor 830 forward and backward by pushing against the motor mount pre-load block 840. The motor mount pre-load block 840 and the motor 830 can be coupled. The motor mount pre-load block 840 and the motor 830 translate within the top housing 802 and the bottom housing 802.

The motor mount pre-load block 840 and the motor 830 can be prevented from rotating relative to the cam bearing block 836. The cam bearing block 836 can remain in position as the motor 830 translates. The motor mount pre-load block 840 and the motor 830 can axially translate relative to the cam bearing block 836. The cam bearing block 836 can be fixed relative to the top housing 802 and the bottom housing 806. The motor mount pre-load block 840 and the motor 830 can axially translate when the adjuster 822 is rotated. The motor mount pre-load block 840 and the motor 830 can axially translate in a first direction to increase the pressure in the system 1100. The motor mount pre-load block 840 and the motor 830 can axially translate in a second direction, opposite the first direction to decrease the pressure in the system 1100. The motor mount pre-load block 840 can allow free rotation of the motor shaft 831. The motor shaft 831 can extend through a bore of the motor mount pre-load block 840. The motor mount pre-load block 840 and the motor 830 can axially translate a predetermined distance. The motor mount pre-load block 840 and the motor 830 can axially translate a set distance that prevents over-pressurization of the system 1100. The motor mount pre-load block 840 can have limited translation relative to the cam bearing block 836. The motor mount pre-load block 840 and the motor 830 can only translate relative to the cam bearing block 836. The motor mount pre-load block 840 and the motor 830 cannot rotate relative to the cam bearing block 836. The motor mount pre-load block 840 and the motor 830 can only translate. The motor mount pre-load block 840 can couple to the motor thrust bearing 872. The motor thrust bearing 872 can translate as the motor 830 translates.

Additional components can translate as the motor 830 translates. The proximal printed circuit board cage 834 translates as the motor 830 translates. The cable strain relief 832 translates as the motor 830 translates. The adjuster 822 can rotate relative to cable strain relief 832. The cable strain relief 832 does not rotate as the adjuster 822 rotates. The cable strain relief 832 only translates as the adjuster 822 translates.

The motor 830 can translate a predetermined distance. The predetermined distance can be related to a gap between the motor mount pre-load block 840 and the cam bearing block 836. The cam bearing block 836 can prevent forward movement of the motor mount pre-load block 840 when surfaces of the cam bearing block 836 and the motor mount pre-load block 840 abut. The motor 830 can translate a predetermined distance that prevents over pressurization of the system 1100. The amplitude of the pressure can correspond to the compression of the spring 846. The motor 830 can translate a predetermined distance that prevents over compression of the spring 846. The motor 830 can translate a predetermined distance within the cam bearing block 836. The motor 830 can translate a predetermined distance related to the compression of the spring 846. The distance that the motor 830 translates can correspond to the maximum and minimum compression of the spring 846. The spring 846 can provide a force onto the motor mount pre-load block 840. The spring 846 can provide a force to abut the motor mount pre-load block 840 and the adjuster 822.

The motor 830 can translate to change the pressure of the device. The motor 830 can translate to compress the spring 846. The motor 830 can translate forward or distally relative to the top housing 802 and the bottom housing 806 to compress the spring 846. The motor 830 can translate backward or proximally relative to the top housing 802 and the bottom housing 806 to decompress the spring 846. The motor 830 can translate a predetermined distance which limits the range of compression of the spring 846. The spring 846 can store compressive energy. The spring 846 can store energy to apply to the cam hammer 848. The cam hammer 848 can apply more energy when the spring 846 is more compressed. The cam hammer 848 can apply less energy when the spring 846 is less compressed. The cam hammer 848 can apply energy depending on the location of the motor 830. The user can rotate the adjuster 822 to change the position of the motor 830. The motor 830 can be coupled to the spline adapter 868. The spline adapter 868 can receive the spring. The user can change the position of the motor 830 to change the position of the spline adapter 868. The spline adapter 868 can translate forward or distally relative to the top housing 802 and the bottom housing 806 to compress the spring 846. The spline adapter 868 can translate backward or proximally relative to the top housing 802 and the bottom housing 806 to decompress the spring 846. The user can rotate the adjuster 822 to compress the spring 846. The user can rotate the adjuster 822 to change the pressure that the cam hammer 848 applies.

The motor 830 can include the motor shaft 831 that rotates at the frequency determined by the speed control 810. Additional components such as the spline adapter 868 can rotate as the motor shaft 831 rotates. The spline adapter 868 rotates as the motor shaft 831 rotates. The cam can 860 rotates as the motor shaft 831 rotates. The cam can insert 862 rotates as the motor shaft 831 rotates. The cam can insert 862 rotates around the cam hammer 848. In some embodiments, the cam hammer 848 does not rotate as the motor shaft 831 rotates. The cam hammer 848 only translates as the motor shaft 831 rotates. In some embodiments, the spring 846 does not rotate as the motor shaft 831 rotates. The spring 846 only translates as the motor shaft 831 rotates. The spring 846 fits within a bore of the spline adapter 848. The spring 846 fits within the cavity 849 of the cam hammer 848. The spring 846 applies a force to both the spline adapter 848 and the cam hammer 848. The spring 846 stores compressive energy between the spline adapter 848 and the cam hammer 848. The cam hammer 848 can receive energy from the spring 846. The cam hammer 848 can transfer energy to the piston 878.

The spring 846 can be loaded and unloaded as the motor shaft 831 rotates. The spring 846 can be loaded and unloaded as the cam surface 850 of the cam hammer 848 interacts with the ramp 864 of the cam can insert 862. The motor shaft 831 of the motor 830 can rotate. The motor shaft 831 can couple to the spline adapter 868. The spline adapter 868 can rotate with the motor shaft 831. The spline adapter 868 can rotate the cam can 860. The cam can 860 can rotate with the motor shaft 831. The cam can 860 can be coupled to the cam can insert 862. The cam can insert 862 can rotate with the motor shaft 831. As the motor shaft 831 rotates, the spline adapter 868, the cam can 860, and the cam can insert 862 rotate.

The generator 800 can include a position sensor system to determine the position of the cam can 860. The position sensor system can be a reflective position sensor system. The system 1100 can monitor rotation of the cam can 860. The cam can 860 can include white reflective indicators on an otherwise black body. The printed circuit board assembly 816 can include a reflective infrared sensor detector. The printed circuit board assembly 816 can position the reflective infrared sensor detector relative to the cam can 860 to monitor the cam can 860. The cam can 860 can provide contrast between reflective indicators and the body of the cam can 860. The reflective indicators can be detected by the reflective infrared sensor detector of the printed circuit board assembly 816 to determine the rotation of the cam can 860. The rotation of the cam can 860 can correspond with the frequency of the pressure wave in some embodiments.

The spline adapter 868 can translate within the cam can 860. The spline adapter 868 can translate when the adjuster 822 translates. The spline adapter 868 can translate forward or distally when the adjuster 822 is rotated in a first direction. The spline adapter 868 can translate forward when the adjuster 822 is translated forward. The spline adapter 868 can translate forward when the motor 830 is translated forward. The spline adapter 868 can compress the spring 846 when the motor 830 is translated forward. The spline adapter 868 can compress the spring 846 such that the spring applies a greater compressive force on the cam hammer 848. The spline adapter 868 can compress the spring 846 to increase the maximum pressure delivered by the generator 800.

The spline adapter 868 can translate backward or proximally when the adjuster 822 is rotated in a second direction, opposite the first direction. The spline adapter 868 can translate backward when the adjuster 822 is translated backward. The spline adapter 868 can translate backward when the motor 830 is translated backward. The spline adapter 868 can decompress the spring 846 when the motor 830 is translated backward. The spline adapter 868 can decompress the spring 846 such that the spring applies a lesser compressive force on the cam hammer 848. The spline adapter 868 can decompress the spring 846 to decrease the maximum pressure delivered by the generator 800 . . . . The spline adapter 868 can translate within the cam can 860. The spline adapter 868 can translate as the motor 830 translates. The motor 830 translates to change the compressive force of the spring 846.

The motor shaft 831, the spline adapter 868, the cam can 860 and the cam can insert 862 can rotate together. The motor shaft 831 can transmit torque to the spline adapter 868. The motor shaft 831 can transmit torque to the cam can 860. The motor shaft 831 can transmit torque to the cam can insert 862. The cam can insert 862 can have the ramp 864 to actuate the cam hammer 848. The cam can insert 862 can rotate relative to the cam hammer 848. The cam can insert 862 can transmit torque from the cam can 860 to the cam hammer 848. The cam hammer 848 can translate as the cam surface 850 interacts with the rotating cam insert 862. The cam insert 862 can rotate about the cam hammer 848. The cam hammer 848 can rise and fall as the cam surface 850 of the cam hammer 848 interacts with the ramp 864 of the cam insert 862.

The cam hammer 848 can rise and fall to impart energy. The cam hammer 848 can transmit energy to the piston 878. The cam hammer 848 can cyclically impact the piston 878. The cam hammer 848 can cyclically impact the piston 878 based on the ramp 864 of the cam can insert 862 interacting with the cam surface 850 of the cam hammer 848. The cam hammer 848 can apply an impact force on the piston 878. The cam hammer 848 can apply an impact frequency to the piston 878 based on the speed of the motor 830. The cam hammer 848 can apply an impact force to the piston 878 based on the compression of the spring 846. The cam hammer 848 can apply a greater impact force to the piston 878 when the spring 846 is more compressed. The cam hammer 848 can apply a lesser impact force to the piston 878 when the spring 846 is less compressed. The cam hammer 848 interfaces with the cam can insert 862 to load and unload the spring 846 as the cam can insert 862 rotates.

The cam hammer 848 can translate proximally and distally. The cam hammer 848 can translate forward and backwards. The cam hammer 848 can translate relative to the hammer bearing block 852. The cam hammer 848 can be prevented from rotating relative to the hammer bearing block 852. The hammer bearing block 852 can allow rotation of the cam can 860. The cam hammer 848 can translate relative to the cam bearing block 836. The hammer bearing block 852 and the cam bearing block 836 can be coupled. The hammer bearing block 852 and the cam bearing block 836 can be fixed relative to the top housing 802 and the bottom housing 806. The cam hammer 848 can translate relative to the top housing 802 and the bottom housing 806. The cam hammer 848 can translate relative to the cylinder 876 and reservoir 854. The cylinder 876 can be coupled to the hammer bearing block 852. The reservoir 854 can be coupled to the cylinder 876. The cylinder 876 and reservoir 876 can be fixed relative to the top housing 802 and the bottom housing 806.

The cam hammer 848 can translate to actuate the piston 878. The cam hammer 848 can actuate the piston 878 a discrete number of times per rotation of the cam can 860 and cam can insert 862. The cam hammer 848 can actuate the piston 878 a discrete number of times per rotation of the motor shaft 831. In some embodiments, the cam hammer 848 can actuate the piston 878 each rotation. In some embodiments, the cam hammer 848 can actuate the piston 878 twice per rotation. In some embodiments, the cam hammer 848 can actuate the piston 878 four times per rotation. In some embodiments, the cam hammer 848 can control the frequency of pulses generated by the generator 800. In some embodiments, the cam can insert 862 can control the frequency of pulses generated by the generator 800. The frequency of pulses generated by the generator 800 can be determined by the speed of the motor 830 and the shape of the ramp 864 of the cam can insert 862. The number of projections of the ramp 864 can increase the frequence of pulses generated per rotation of the motor shaft 831. The frequency of movement of the cam hammer 848 can correspond to the movement of the piston 878. The frequency of movement of the cam hammer 848 can correspond to the movement of the membrane 884. The cam hammer can translate relative to the membrane 884.

The piston 878 can translate proximally and distally. The piston 878 can translate forward and backwards. The piston 878 can translate within the cylinder 876. The piston 878 can include a surface that abuts with the cylinder 876 to prevent the piston 878 from being retracted too far. The piston 878 can translate relative to the cylinder 876. The piston 878 can be prevented from rotating relative to the cylinder 876. The cylinder 876 can allow rotation of the piston 878. The piston 878 can translate relative to the reservoir 854. The cylinder 876 and the reservoir 854 can be coupled. The cylinder 876 and the reservoir 854 can be fixed relative to the top housing 802 and the bottom housing 806. The piston 878 can translate relative to the top housing 802 and the bottom housing 806. The piston 878 can translate relative to the membrane 884. The membrane 884 can be coupled to the cylinder 876. The membrane 884 can be coupled to the reservoir 854. The membrane 884 can be deflected relative to the cylinder 876 and the reservoir 854. The piston 878 can couple to the piston o-ring 880. The piston o-ring 880 can seal an interface between piston 878 and the cylinder 876. The piston 878 can have forward travel. The forward travel of the piston 878 can be accommodated by the deflecting membrane 884. The backward travel of the piston 878 can be limited by the cylinder 876.

The piston 878 can transfer impact energy from the cam hammer 848 to the membrane 884. The piston 878 can deflect the membrane 884. The piston 878 can include materials that reduces the impact of sound. The piston 878 can include sound dampening materials, such as a thermoplastic polyurethane (TPU) overmold or the addition of an o-ring, such as nitrile, or an fluoroelastomer, at the surface of the piston-hammer contact. The piston 878 can include an overmolded structure. The piston 878 can include glass filled nylon, a single metal, such as aluminum or titanium, a composite metal, such as stainless steel or Nitinol, a infused polymer, such as a carbon nano-tube infused. The piston 878 can apply a greater impact force to the membrane 884 when the spring 846 is more compressed. The piston 878 can apply a lesser impact force to the membrane 884 when the spring 846 is less compressed. The membrane 884 can be between the reservoir 854 and the cylinder 876. The membrane 884 can create a fluid seal between the cylinder 876 and the reservoir 854. The membrane 884 can allow the piston 878 to transfer impact energy to fluid inside the reservoir 854. The membrane 884 can elongate to allow piston 878 to translate. The pressure of the reservoir 854 can be monitored by the pressure sensor 888.

The deflection of the membrane 884 can create the pressure wave. The deflection of the membrane 884 can create the pressure wave with a variety of frequencies. The deflection of the membrane 884 can create the acoustic wave. The deflection of the membrane 884 can create the fluid wave. The pressure wave can propagate through the liquid medium used to fill the balloon 100. The frequencies carried on the pressure wave can be a single frequency. The frequencies carried on the pressure wave can be more than one discrete frequency ranges. Independent of the frequencies carried on the pressure wave, the pressure waves can have some level of attenuation to the surrounding tissue by exiting through the balloon body and out the surface of the balloon 100. The pressure wave can propagate from the balloon to the tissue. In some embodiments, the production of pressure waves carry a broad range of frequencies with some frequencies having higher amplitude than others. The pressure waves can carry acoustic frequencies. The amplitude and frequencies of the pressure waves can be tuned to increase the amplitude of some frequencies while minimizing the amplitude of other frequencies. Frequencies in the low frequency range, such as the Solfeggio frequencies including 174 Hz, 285 Hz, 396 Hz, 417 Hz, 528 Hz, 639 Hz, 741 Hz, 852 Hz, and 963 Hz, can reduce cellular damage and increase cellular healing. Although the Solfeggio frequencies are considered, the use of these specific frequencies can be captured here to emphasize the opportunity of frequencies throughout the acoustic spectrum that are considered for increasing cellular repair since the pulse enhanced serration balloon system can be capable of delivering a broad range of frequencies from 20 Hz and below to 1 MHz and above through any lumen to deliver a specified energy locally.

In some embodiments, the base pressure of the balloon 100 can be set prior to the generator 800 being actuated, such as by the inflation device 400 connected to the reservoir 854. The fluid in the reservoir 854 can be at the base pressure. The system 1100 can be configured to produce the pressure wave. The system 1100 can return to the base pressure after each pressure wave. The base pressure can serve as a back pressure for resetting the system 1100. The base pressure can act on the system to retract the membrane 884. The base pressure resets the membrane 884 to its initial starting position after the pressure wave is sent. The system 1100 can return to the base pressure by resetting the membrane 884. The base pressure can act on the system to retract the piston 878. The base pressure resets the piston 878 to its initial starting position after the pressure wave is sent. The system 1100 can return to the base pressure by resetting the piston 878. The base pressure can act on the system 1100 to retract the cam hammer 848. The base pressure resets the can hammer 848 to its initial starting position after the pressure wave is sent. The system 1100 can return to the base pressure by resetting the cam hammer 848. The base pressure resets the system 1100 to the neutral position wherein the generator is ready to produce the pressure wave. The base pressure resets the can spring 846 to its initial starting position after the pressure wave is sent. The system 1100 can return to the base pressure by the force provided by the spring 846.

The system 1100 can be configured to produce the pressure wave. The movement of the membrane 884 can cause the pressure wave. The amplitude of pressure generated by the system 1100 can be increased by increasing the force of the piston 878 striking the membrane 884 by increasing the compression of the spring 846 . . . . The amplitude of pressure generated by the system 1100 can be decreased by decreasing the force of the piston 878 striking the membrane 884 by decreasing the compression of the spring 846. The amplitude of pressure generated by the system 1100 can be decreased by decreasing the force of the piston 878 striking the membrane 884 and reducing the volume of liquid displaced by the membrane 884. The amplitude of pressure generated by the system 1100 can be decreased by decreasing the force of the piston 878 striking the membrane 884 and reducing the travel of the piston 878 against the membrane 884. The pressure wave can vary in pressure between the base pressure and the pressure spike. The pressure range between the base pressure and the pressure spike can be from 2 atm to 8 atm, from 2 atm to 10 atm, from 2 atm to 12 atm, from 2 atm to 14 atm, from 2 atm to 16 atm, from 2 atm to 18 atm, from 2 atm to 20 atm, or any range of two of the foregoing values. The pressure difference between the base pressure and the pressure spike can be 4 atm, 6 atm, 8 atm, 10 atm, 12 atm, 14 atm, 16 atm, 18 atm, 20 atm, or any range of two of the foregoing values. The pressure wave can vary in pressure. The pressure wave can have a single peak pressure. The pressure wave generated by the system 1100 can have a pressure measured at the balloon 100. The pressure wave generated by the system 1100 can have a pressure measured within the liquid medium used to fill the balloon 100. The pressure wave generated by the system 1100 can have a pressure measured at the reservoir 854. The pressure wave generated by the system 1100 can have a pressure measured by the pressure sensor 888. The pressure wave generated by the system 1100 can have a pressure measured in the proximal handle of the generator 800. The pressure wave generated by the system 1100 can be amplified by the raised features 300 of the strip 200. The pressure can be measured within the tissue. The pressure can be amplified within the tissue compared to the pressure inside the balloon 100. The pressure wave can be a shock wave. The pressure wave can travel faster than the speed of sound. The pressure waves create a sudden discontinuous jump in pressure with the fluid of the system 1100. The pressure wave can require an initial energy discharge. The energy discharge can be the striking of the membrane 884. The striking of the membrane 884 can produce a shock wave. The shock wave produced by the system 1100 does not require an electrical discharge. The shock wave produced by the system 1100 does not require a laser pulse. The shock wave produced by the system 1100 does not require a collapsing cavitation bubble. The shock wave can be produced by the generator 800, as described herein.

The pressure wave can propagate through liquid medium. The pressure wave can propagate through liquid medium in the reservoir 854. The pressure wave can travel the length of the catheter 600 to the balloon 100. The pressure wave can propagate through liquid medium in the balloon 100. The pressure wave can originate proximal to the proximal end of the balloon 100. The pressure wave can travel without decreasing in pressure along the length of the balloon. In some embodiments, the pressure wave may decrease in pressure as the pressure wave travels distally. As the pressure wave travels, a change in pressure can be detected. The pressure wave can travel at the speed of sound. The pressure wave can travel as a fluid wave along the length of the balloon. The pressure wave can also be an acoustic wave. The pressure wave can carry a variety of acoustic frequencies. The pressure wave can carry energy and information through the liquid medium. In some embodiments, the pressure wave can steepen as the pressure waves travels through the liquid medium. The pressure wave can have any waveform described herein. The pressure wave can be a longitudinal pressure wave, such as a sound wave. The pressure wave can consist of high pressure compressions and low pressure rarefactions. The compressions travel faster than the rarefactions causing the waveform to become steeper. The pressure increases nearly instantaneously at the wave front, resulting in a shock wave. In some embodiments, the shock wave occurs over the length of the balloon 100. In some embodiments, the shock wave occurs over a length greater than the length of the balloon 100. For instance, the pressure wave can be detected past the distal end of the balloon 100 with some energy emitted past the balloon body and out the distal end of the balloon 100.

The pressure wave can move from the reservoir 854 to the balloon 100. The pressure waves can move through the liquid medium used to fill the catheter 600. The pressure waves can travel from the liquid medium of the balloon 100 and through the wall of the balloon 100. The pressure wave can travel from the liquid medium of the balloon 100 to the strip 200 and the raised feature 300. The pressure wave can travel through each medium delivering a proportional component of the wave energy relative to the reflection coefficient of each medium that the pressure wave passes through. The membrane 884 can be configured to be deflected. The deflection of the membrane 884 can produce a volumetric change in the liquid medium. The volumetric change can expand the balloon 100. The balloon 100 can have a set volume when inflated. The membrane 884 can be deflected to cause a volumetric change. The membrane 884 can be deflected to cause a pressure change. The membrane 884 can be deflected to nearly match the growth of the balloon at both the upper and lower boundaries of pressure corresponding to the compliance curves of the balloon. The membrane 884 can be deflected to induce a pressure change from about 2 atm to about 10 atm. The membrane 884 can be deflected to induce a pressure change of between 4 atm and 8 atm. The raised features 300 of the strip 200 can act like magnifying points for the pressure wave. The raised features 300 of the strip 200 can magnify the pressure wave by 1000× to 1500×. This large magnification of the pressure wave can accelerate the speed at which fracturing and vessel compliance occurs while still maintaining a propensity for the fracture to occur in one or more longitudinally oriented lines. The pressure wave can be accelerated by the shape of the balloon 100. The proximal cone of the balloon 100 can accelerate the speed of the pressure wave. The body of the balloon 100 can maintain the speed of the pressure wave along the length of the balloon 100. The distal cone of the balloon 100 can deaccelerate the speed of the pressure wave.

The system 1100 can include the generator 800. The generator 800 can be external to the balloon 100. The generator 800 can generate pressure waves. The pressure waves can be sent from the reservoir 854 to the balloon 100. The energy from the pressure wave can be rapidly absorbed by the fluid medium within the balloon 100. The energy from the pressure wave can be rapidly absorbed from the proximal end of the balloon 100 to the distal end of the balloon 100. The energy from the pressure wave can be rapidly absorbed over the entire length of the balloon 100. The pressure wave can propagate through the balloon 100 to create bursts of pressure. The pressure wave can propagate through the balloon 100 at ultrasonic frequencies The pressure wave can propagate through the balloon 100 and enter surrounding tissue.

The pressure wave within the fluid of the system 1100 can cause a pressure wave that extends beyond the distal end of the balloon 100. The pressure wave can emanate radially from the balloon 100. The pressure wave can emanate longitudinally from the balloon 100. The pressure wave can emanate from the one or more strips 200. The pressure wave can emanate from the raised features 300. The pressure wave can produce a lithotripsy effect. The pressure wave can impinge on the vessel to break calcium bonds. The pressure wave can impinge on the vessel to break mineralized calcium bonds. The pressure wave can propagate from the balloon 100. The pressure wave can propagate through the lobes 120 of the balloon 100. The pressure wave can propagate through the strips 200. The pressure wave can propagate through the raised features 300. The pressure wave can travel through the vessel. The pressure wave can fracture the hardened calcium deposits. The pressure wave can break calcium bonds. In some embodiments, the pressure wave does not damage healthy tissue. In some embodiments, the pressure waves only breaks calcified regions. The pressure waves can allow the vessel to become more compliant as described herein. The pressure waves can allow the vessel to become more flexible. The pressure waves can break calcium bonds to achieve higher volume flow rates as described herein. The pressure waves can break calcium bonds to improve compliance. The pressure waves can break calcium bonds to improve flow dynamics. The pressure waves can break calcium bonds to increase the internal diameter of the vessel. The pressure waves can break calcium bonds to prevent recoil. The pressure waves can break calcium bonds to allow plaque to become less turbulent. The pressure waves can break calcium bonds enabling more effective displacement of the plaque resulting in less turbulent flow. The pressure waves can break calcium bonds to allow plaque to have fewer perturbances in the flow stream. The pressure waves can break calcium bonds to reduce wall friction. The pressure waves can break calcium bonds to reduce flow resistance. The pressure waves can break calcium bonds to increase the vessel size to increase blood flow. The pressure waves can break calcium bonds to increase lumen gain. The pressure waves can break calcium bonds to reduce recoil. The pressure waves can break calcium bonds within the cellular network of the vessel wall. The pressure waves can break calcium bonds in a permanent manner. The pressure waves can break calcium bonds to permanently remodel the vessel. In some embodiments, the pressure waves can only create a shock wave. In some embodiments, the pressure wave can cause only lithotripsy effects. In some embodiments, the generator 800 can only produce pressure waves that have a lithotripsy effects. In some embodiments, the generator 800 can only produce shock waves.

The pressure waves can create serrations, indentations, and/or microperforations. The pressure waves can expand the balloon 100. The pressure waves can cause radial displacement of the strip 200 and the raised features 300. The pressure waves can move the strip 200 and the raised features 300 radially outward. The pressure waves can allow the raised features 300 to apply a force toward the vessel wall. The pressure waves can increase and decrease the pressure within the balloon 100. The pressure waves can cause outward movement of the strip 200 and the raised features 300. The pressure waves can cause the raised features 300 to create serrations, indentations, and/or microperforations in the surface of the plaque. The pressure waves can cause the raised features 300 to create serrations, indentations, and/or microperforations in the internal elastic lamina. The pressure waves can cause the raised features 300 to create a line of weakness in the plaque. In some embodiments, the pressure waves can only create serrations, indentations, and/or microperforations. In some embodiments, the pressure waves can create serrations, indentations, and/or microperforations and create a shock wave. In some embodiments, the pressure waves can cause both serrations and lithotripsy effects.

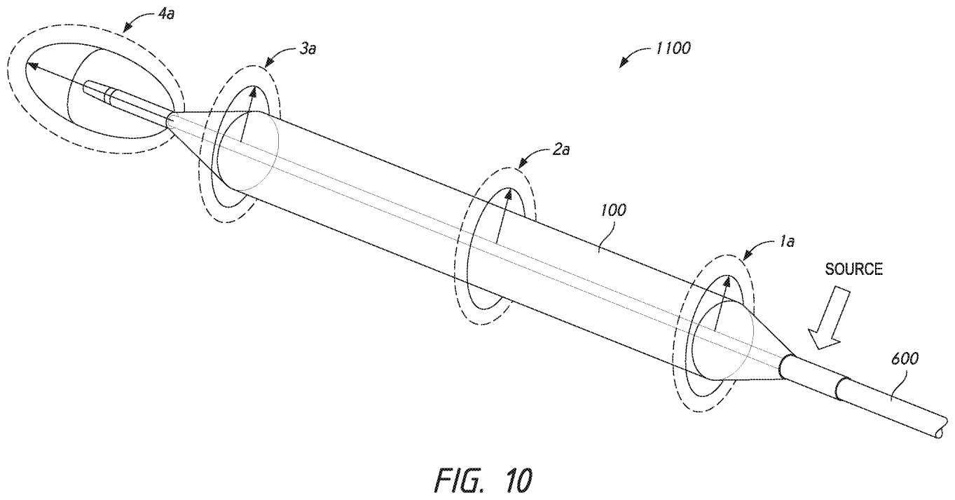

The system 1100 can produce a series of pressure waves. Before, after and between the series of pressure waves, the system 1100 can return to a baseline, low pressure. Each pressure wave of the series of pressure waves can spike to the same maximum pressure. Each pressure wave, in the series of pressure waves, can be configured to have an upper limit for the maximum pressure. Each pressure wave of the series of pressure waves can spike to different maximum pressures. Each pressure wave of the series of pressure waves can have a maximum pressure of 4 atm, 5 atm, 6 atm, 7 atm, 8 atm, 9 atm, 10 atm, 11 atm, 12 atm, 13 atm, 14 atm, 15 atm, 16 atm, 17 atm, 18 atm, 19 atm, 20 atm, or any range of two of the foregoing values. The series of pressure waves can have two pressure waves with a pressure difference between maximum pressures of 4 atm, 5 atm, 6 atm, 7 atm, 8 atm, 9 atm, 10 atm, 11 atm, 12 atm, 13 atm, 14 atm, 15 atm, 16 atm, 17 atm, 18 atm, 19 atm, 20 atm, or any range of two of the foregoing values. The pressure waves can propagate from theoretical source proximal to the proximal end of the balloon 100. The theoretical source can be within the catheter 600. The theoretical source can be within the generator 800. The theoretical source can be the reservoir 854. An example of the theoretical source can be shown in FIG. 10. The pressure waves can propagate from outside of the body of the balloon 100. The pressure waves can propagate outside of the body of the balloon 100 while introduced intravascularly through the balloon 100.

### Wave Parameters and Waveforms

The system 1100 can produce pressure waves with key technical parameters to allow lithotripsy. The system 1100 can produce pressure waves that are highly controlled. The system 1100 can produce pressure waves that are delivered with the generator 800. The system 1100 can produce pressure waves that are delivered to the balloon 100 with strips 200 having raised features 300. While not to be limited by a theory, certain combination of parameters may lead to the unexpected results and advantages described herein. This section describes certain parameters of the systems described herein. This section also describes certain conditions of the balloon 100 with the strips 200 and the raised features. In some embodiments, there can be a combination of parameters that provide unexpected results.

The pressure waves can be generated when the balloon 100 contacts the vessel wall. The pressure waves can be generated when the balloon 100 is filled to contact nearly the entire circumference of the vessel wall. The balloon 100 can include lobes 120, with a corresponding lobe between adjacent strips 200. The pressure waves can be generated when the lobes of the balloon 100 contacts the vessel wall. FIGS. 58C and 58D describe the filling of the lobes 120 to contact the vessel wall. The base pressure can allow the lobes 120 of the balloon 100 to conform to the vessel. The pressure waves can be generated when the raised features 300 contacts the vessel wall. The pressure waves can be generated when the raised features 300 do not penetrate the vessel wall. The pressure waves can be generated when the raised features 300 penetrate the vessel wall to create microperforations. The system 1100 can provide pressure waves along the entire circumference of the ballon 100. The system 1100 can provide lithotripsy through the lobes 120 of the balloon 100. The system 1100 can provide lithotripsy through the strips 200 with the raised features 300. The system 1100 can provide lithotripsy along the entire circumference of the ballon 100. The system 1100 can provide lithotripsy along the entire length of the balloon 100. The system 1100 can provide lithotripsy greater than the length of the balloon 100.

The pressure wave can be generated when the balloon 100 is inflated to the base pressure. The base pressure can be the starting fluid system pressure. The base pressure can be the base pressure of the reservoir 854. The base pressure can be the base pressure of the catheter 600. The base pressure can be the base pressure of the balloon 100. The base pressure can be established prior to initiating the pressure waves. The pressure in the system 1100 can alternate between the base pressure and the peak positive pressure during the pressure wave generation. The base pressure can be 2 ATM, 2.5 ATM, 3 ATM, 3.5 ATM, 4 ATM, 4.5 ATM, 5 ATM, 5.5 ATM, 6 ATM, 6.5 ATM, 7 ATM, 7.5 ATM, 8 ATM, between 2 ATM and 8 ATM, between 2 ATM and 4 ATM, or any range of two of the foregoing values. The base pressure can start at 2 ATM. The lower limit of the base pressure can be 0 ATM, 0.5 ATM, 1 ATM, 1.5 ATM, 2 ATM, 2.5 ATM, 3 ATM, 3.5 ATM, 4 ATM, or any range of two of the foregoing values. The upper limit of the base pressure can be 4 ATM, 4.5 ATM, 5 ATM, 5.5 ATM, 6 ATM, 6.5 ATM, 7 ATM, 7.5 ATM, 8 ATM, 8.5 ATM, 9 ATM, 9.5 ATM, 10 ATM, 10. 5 ATM, 11 ATM, 11.5 ATM, 12 ATM, or any range of two of the foregoing values. The base pressure can range from 0 ATM to 8 ATM. The balloon 100 can have a base pressure under 4 ATM. The base pressure can be a sub-nominal pressure. The base pressure can be static, resting pressure. The base pressure can be same at the generator 800 and at the balloon 100. In some embodiments, the base pressure can be controlled by the user. In some embodiments, the base pressure is controlled by the system 1100. In some embodiments, the base pressure can be maintained within a range of 0 ATM to 8 ATM. In some embodiments, both the lobes 120 of the balloon 100 and the raised features 300 contact the vessel wall at the base pressure.

In some embodiments, it can be advantageous for both the lobes 120 of the balloon 100 and the raised features 300 to contact the vessel wall for pressure wave delivery. While not to be limited by a theory, contact of both the lobes 120 and the raised features 300 can ensure efficient energy transfer to the vessel wall for crack propagation and lithotripsy. The contact of both the lobes 120 and the raised features 300 can reduce air gaps which may reflect energy. The contact of both the lobes 120 and the raised features 300 can minimize energy loss. The contact of both the lobes 120 and the raised features 300 can allow the pressure waves to be directed through tissue. The contact of both the lobes and the raised features can ensure deep penetration of the pressure waves to at least the medial layer. The contact of both the lobes 120 and the raised features 300 can allow for the effect treatment of targeted areas. The contact of both the lobes 120 and the raised features 300 can prevent distortion of the waveform of the pressure wave. The contact of both the lobes 120 and the raised features 300 can ensure that the ultrasonic signal remains consistent.

The system 1100 can include the generator 800 that produces the pressure wave. The system 1100 can have pressure generator parameters controlled by the user. The system 1100 can have a peak positive pressure. The peak positive pressure can be generated by the pressure wave. The peak positive pressure can be the maximum fluid system pressure. The pressure wave can cause a peak positive pressure. The peak positive pressure can be the pressure spike experienced by the balloon 100 when the pressure wave is generated. The peak positive pressure can be maintained below a threshold. The peak positive pressure can be maintained below the threshold during the procedure. The peak positive pressure can be adjusted by the user. The user can adjust the adjuster 822 to change the compression of the spring 846. The user can move the adjuster 822 toward the reservoir 854 to increase the compression of the spring 846. The user can move the adjuster away from the reservoir 854 to decrease the compression of the spring 846. The compression of the spring 846 can be correlated with the peak positive pressure of the fluid of the system 1100. The increase in compression of the spring 846 can cause a greater peak positive pressure. The decrease in compression of the spring 846 can cause a lesser peak positive pressure. The user can adjust the peak positive pressure within a range. The user can adjust the peak positive pressure continuously along a range based on the position of the adjuster 822. The user can adjust the peak positive pressure continuously along a range based on compression of the spring 846. The system 1100 can include stops that limit the translation of the adjuster 822. The system 1100 can include stops that limit the compression of the spring 846. These stops can also limit the range of the peak positive pressure delivered by the system 1100.

The peak positive pressure can be the peak positive pressure of the reservoir 854. The peak positive pressure can be the peak positive pressure of the catheter 600. The peak positive pressure can be the peak positive pressure of the balloon 100. The peak positive pressure can be the maximum pressure during the pressure wave. The peak positive pressure can be the maximum pressure during the activation of the generator 800. The peak positive pressure can be the maximum pressure during the pressure impulses. The peak positive pressure can be the intentional maximum fluid system pressure during activation of the generator 800. In some embodiments, the peak positive pressure can be independent of the base pressure.

The peak positive pressure can be 8 ATM, 8.5 ATM, 9 ATM, 9.5 ATM, 10 ATM, 10. 5 ATM, 11 ATM, 11.5 ATM, 12 ATM, or any range of two of the foregoing values. The peak positive pressure can about 9 ATM. The lower limit of the peak positive pressure can be 4 ATM, 4.5 ATM, 5 ATM, 5.5 ATM, 6 ATM, 6.5 ATM, 7 ATM, 7.5 ATM, 8 ATM, 8.5 ATM, 9 ATM, 9.5 ATM, 10 ATM, or any range of two of the foregoing values. The upper limit of the peak positive pressure can be 14 ATM, 14.5 ATM, 15 ATM, 15.5 ATM, 16 ATM, 16.5 ATM, 17 ATM, 17.5 ATM, 81 ATM, 18.5 ATM, 19 ATM, 19.5 ATM, 20 ATM, 20. 5 ATM, 21 ATM, 21.5 ATM, 22 ATM, 22.5 ATM, 23 ATM, 23.5 ATM, 24 ATM, 24.5 ATM, 25 ATM, or any range of two of the foregoing values. The peak positive pressure can range from 5 ATM to 20 ATM. The peak positive pressure at the generator 800 can be different than the peak positive pressure at the balloon 100. In some embodiments, some energy can be dissipated along the catheter 600 before the energy reaches the balloon 100. The peak positive pressure can be lower at the balloon 100. The peak positive pressure can be maintained below a threshold. The peak positive pressure can be maintained below 20 ATM. The peak positive pressure can be maintained below 10 ATM. The peak positive pressure can be maintained below a threshold of 9 ATM, 10 ATM, 11 ATM, 12 ATM, 13 ATM, 14 ATM, 15 ATM, 16 ATM, 17 ATM, 18 ATM, 19 ATM, 20 ATM, or any range of two of the foregoing values.

The peak positive pressure can be the highest compression point. The peak positive pressure can characterize the pressure wave generated by the generator 800. The peak positive pressure can characterize the pressure wave generated by the generator 800 and measurable within the balloon 100. The peak positive pressure of the pressure wave can be a single maximum value. The peak positive pressure of the pressure wave can be the maximum pressure amplitude. The peak positive pressure of the pressure wave can be a range of values. The peak positive pressure can be correlated with the strike force of the piston 878. The peak positive pressure can be correlated with the compression of the spring 846. The peak positive pressure can be correlated with the deflection of the membrane 884. In some embodiments, the membrane 884 can be unmovable above the peak positive pressure. In some embodiments, the system 1100 can be constrained to deliver the peak positive pressure below a threshold. In some embodiments, the peak positive pressure can be controlled by the user. In some embodiments, the peak positive pressure is controlled by the system 1100. In some embodiments, the peak positive pressure can be maintained within a range of 5 ATM to 20 ATM. In some embodiments, the peak positive pressure is about 9 ATM. In some embodiments, the peak positive pressure can be maintained within a range of 5 ATM to 15 ATM. In some embodiments, the peak positive pressure can be under 15 ATM. The peak positive pressure can be generally low pressure inflation compared with other angioplasty techniques which frequently exceed 15 ATM. The peak positive pressure can ensure that the balloon 100 does not exceed diameter limits. The peak positive pressure can be a relatively low range, preventing balloon rupture.

In some embodiments, it can be advantageous for the peak positive pressure to be within the range described herein. While not to be limited by a theory, the peak positive pressure of the system 1100 described herein can prevent vessel trauma. The peak positive pressure can reduce the risk of intimal tearing. The peak positive pressure can prevent random cracking. The peak positive pressure can reduce dissections. The peak positive pressure of the system 1100 described can be transient. The peak positive pressure can be the peak pressure imparted by the pressure wave. The balloon 100 can return to the base pressure almost instantaneously based on speed of the pressure wave. While not to be limited by a theory, the system 1100 can be the low, base pressure during the majority of the procedure, wherein using low pressure can be termed gentle angioplasty. In typical angioplasty, peak positive pressure of greater than 15 ATM can be used to provide the necessary force over a longer duration to compress the plaque. The balloon is inflated and maintained at the peak positive pressure to try to remodel the vessel. In contrast, the fluid in the system 1100 experiences the peak positive pressure in fleeting spurts. The peak positive pressure can travel with the pressure wave at the speed of the sound. The fluid in the system 1100 nearly instantaneously increase to the peak positive pressure and nearly instantaneously returns to the base pressure.

The system 1100 can have one or more pressure generator parameters controlled by the user. The system 1100 can have a pressure wave frequency. The system 1100 can have a rate at which the pressure waves are generated. The system 1100 can have a waveform frequency. The system 1100 can have a measurement of time between pressure waves. The system 100 can have a pulse frequency. The pulse frequency can be a pulse repetition frequency. The pulse frequency can be how many pressure waves occur per second. The pulse frequency can be 0.5 Hz. The pulse frequency can be 1 Hz. The pulse frequency can be 2 Hz. The pulse frequency can be 3 Hz. The pulse frequency can be 4 Hz. The pulse frequency can be 5 Hz. The pulse frequency can be selected from the group of 1 Hz, 2 Hz, or 3 Hz. The pulse frequency can be selected from the group of 0.5 Hz, 1 Hz, 2 Hz, 3 Hz, or 4 Hz. The pulse frequency can be selected from the group of 0.5 Hz, 1 Hz, 2 Hz, 3 Hz, 4 Hz, and 5 Hz. The pulse frequency can be 0.5 Hz, 1 Hz, 2 Hz, 3 Hz, 4 Hz, or 5 Hz. The pulse frequency can be less than 1 Hz. The pulse frequency can be 3 Hz or less. The pulse frequency can be less than 4 Hz. The pulse frequency can be less than 5 Hz. The pulse frequency can be less than 10 Hz. The pulse frequency can be less than 100 Hz. The pulse frequency can be less than 1 kHz. The pulse frequency can be maintained below a threshold. The pulse frequency can be maintained below 5 Hz. The peak positive pressure can be maintained between 1 Hz and 3 Hz. The pulse frequency can be maintained below a threshold of 5 Hz. The pulse frequency of the pressure wave can be increased by increasing the speed of the motor 830. The pulse frequency of the pressure wave can be increased by decreasing the diameter of the cam can 860. The pulse frequency of the pressure wave can be decreased by decreasing the speed of the motor 830. The pulse frequency of the pressure wave can be decreasing by increasing the diameter of the cam can 860.

The system 1100 can include a pulse-to-pulse frequency. The system 1100 can have an average difference or change in the frequency between consecutive frequency. The pulse-to-pulse frequency can measure the stability or precision of multiple pressure waves. The pulse-to-pulse frequency can be a measurement of variation between consecutive pulses. The system 1100 operating at low pulse frequency can have minimal dynamic error. The system 1100 operating at low pulse frequency can have minimal vibration. The system 1100 operating at low pulse frequency can have minimal mechanical wear. The system 1100 operating at low pulse frequency can have minimal heat signature. The system 1100 operating at low pulse frequency can have consistent accuracy.

The pulse frequency can be maintained below a threshold. The pulse frequency can be maintained below the threshold during the procedure. The pulse frequency can be adjusted by the user. The user can adjust the speed control 810 to change the speed of the motor 830. The user can adjust the speed control 810 to change the rotation of the motor shaft 831 of the motor 832. The user can move the speed control 810 between discreet frequencies. The user can move the speed control 810 between three discreet frequencies. The user can move the speed control 810 to change the pulse frequency from 1 Hz to 2 Hz, or vice versa. The user can move the speed control 810 to change the pulse frequency from 2 Hz to 3 Hz, or vice versa. The position of the speed control 810 can change the speed of the motor 830. As described herein, the motor 830 can cause rotation of the cam can 860 which actuates the cam hammer 848 a certain number of times per rotation based on the ramp 864. In the illustrated embodiment, the motor 830 is rotating at about 0.3 Hz with the pulse frequency 1 Hz. In the illustrated embodiment, the motor 830 is rotating at about 0.6 Hz with the pulse frequency 2 Hz. In the illustrated embodiment, the motor 830 is rotating at about 1 Hz with the pulse frequency 3 Hz. The user can move speed control 810 to increase the pulse frequency. The user can move speed control 810 to decrease the pulse frequency. The user can adjust the pulse frequency within a range. The user can adjust the pulse frequency at discrete steps along a range based on the position of the speed control 810. The system 1100 can include stops that limit the movement of the speed control 810. The system 1100 can include stops that limit the speed of the motor 830. These stops can also limit the range of the pulse frequency delivered by the system 1100. In some embodiments, the system 1100 can minimize the revolutions per minute of the motor 830. In some embodiments, the system 1100 can use a geared 12000 RPM motor 830. The system 1100 can use a planetary gear box to gear down the revolutions per minute at a 138:1 ratio. This ratio means that the input shaft of the motor rotates 138 times for each rotation of the output shaft. The planetary gear box can provide a high reduction ratio. This high reduction ratio significantly increases the torque. This high reduction ratio can drastically reduce speed. The system 1100 can gear down below 138:1 ratio. The system 1100 can gear up above 138:1 ratio. The system 1100 can be designed for applications requiring high force. The system 1100 can be designed for precision motor control.

In some embodiments, it can be advantageous for the pulse frequency to be within the range described herein. While not to be limited by a theory, the pulse frequency of the system 1100 described herein can allow undulation of the strip. The frequency can allow the strip 200 to rise and fall with the fluid wave. The pulse frequency can allow a time period for undulation to occur. The pulse frequency can allow the raised features to zipper into and out of the vessel wall. While not to be limited by a theory, the pulse frequency can allow the system 1100 to reset after each pressure wave. The pulse frequency can allow the fluid to return to the base pressure. While not to be limited by a theory, the pulse frequency of the system 1100 described herein can allow for the formation of cavitation bubbles. The pulse frequency can allow bubbles to form as the raised features zipper into and out of the vessel wall. While not to be limited by a theory, the pulse frequency of the system 1100 can impart periodic shear stresses on calcium to aid in lithotripsy. The pulse frequency can trigger a calcium cellular response. The pulse frequency impact events at a given periodicity function as an acute pressure event at the tissue interface. The pulse frequency impact events at a given periodicity function as acute pressure events resonating through the disease. The pulse frequency impact events at a given periodicity function as acute pressure events resonating through the obstruction. The pulse frequency impact events at a given periodicity function as acute pressure events resonating through the plaque. The pulse frequency impact events at a given periodicity function as acute pressure events resonating through the calcified plaque. The pulse frequency impact events at a given periodicity functions as shock events at the tissue interface. The pulse frequency impact events at a given periodicity function as shock events resonating through the disease. The pulse frequency impact events at a given periodicity function as shock events resonating through the obstruction. The pulse frequency impact events at a given periodicity function as shock events resonating through the plaque. The pulse frequency impact events at a given periodicity function as shock events resonating through the calcified plaque. The pulse frequency can deliver controlled, measured impulses. The pulse frequency can allow for dynamic loading on the calcium.

In some embodiments, it can be advantageous to provide an operating envelope for the user. The system 1100 can define permissible boundaries for certain parameters. The system 1100 can define a range of speeds provided by the motor 830. The defined range of speeds can correlated with the pulse frequency of the pressure waves generated by the system 1100. The system 1100 can define a range of positions of the adjuster 822 which correlates with a defined range of compression of the spring 846. The defined range of compression can correlate with the peak positive pressure of the system 1100. In some embodiments, the user can select from a range of pulse frequencies, wherein all pulse frequencies that can be selected by the user can produce the outcomes described herein. In some embodiments, the user can select from a range of peak positive pressure, wherein all peak positive pressures that can be selected by the user can produce the outcomes described herein.

In some embodiments, the combination of parameters related to pulse frequency and peak positive pressure can allow for a measurable debulking of vascular plaque. In some embodiments, the combination of parameters related to pulse frequency and peak positive pressure can allow for a measurable remodeling of vascular plaque. While not to be bound by a theory, the pulse frequency can control the periodicity of the pressure wave delivery and the peak positive pressure can control the amplitude of the pressure wave delivery. These two parameters relate to both the time domain and the force domain of calcium fracturing. In some embodiments, the combination of parameters within the ranges of the pulse frequency and peak positive pressure can cause production of longitudinal cracks by reducing plaque material. In some embodiments, the combination of parameters within the ranges of the pulse frequency and peak positive pressure can reduce the restrictive layer of the vessel. In some embodiments, the combination of parameters within the ranges of the pulse frequency and peak positive pressure can allow for vessel expansion along longitudinal cracking.

The system 1100 can have a dwell time. The dwell time of the hammer-piston system 848, 878 against the membrane 884 at any given intensity can be altered by the pulse frequency (e.g., the 1 Hz, 2 Hz, or 3 Hz controlled by the speed control 810). The dwell time can be the length of time of contact with the membrane 884. The dwell time can measure the duration of the interaction between the membrane 884 and the hammer-piston system 848, 878. It has been observed that at 3 Hz that the dwell time of the system 1100 at a given intensity is between 55 milliseconds to 65 milliseconds. It has been observed that at 2 Hz that the dwell time of the system 1100 at a given intensity is between 65 milliseconds to 75 milliseconds. It has been observed that at 1 Hz that the dwell time of the system 1100 at a given intensity is between 80 milliseconds to 90 milliseconds. For example for a 2 mm by 20 mm balloon 100, wherein the pulse frequency can be 1 Hz, 2 Hz, or 3 Hz, the dwell times were measured at maximum intensity based on the compression of the spring 846. At 3 Hz, the dwell time from peak to ramp down was 60 milliseconds. At 2 Hz, the dwell time from peak to ramp down was 70 milliseconds. At 1 Hz, the dwell time from peak to ramp down was 84 milliseconds. The decrease in the pulse frequency can increase the dwell time. The increase in the pulse frequency can decrease the dwell time. The dwell time can be 30 milliseconds, 35 milliseconds 40 milliseconds, 45 milliseconds, 50 milliseconds, 55 milliseconds, 60 milliseconds, 65 milliseconds, 70 milliseconds, 75 milliseconds, 80 milliseconds, 85 milliseconds, 90 milliseconds, 95 milliseconds, 100 milliseconds, 105 milliseconds, 110 milliseconds, 120 milliseconds, 125 milliseconds, between 55 milliseconds and 65 milliseconds, between 65 milliseconds and 75 milliseconds, between 80 milliseconds and 90 milliseconds, or any range of two of the foregoing values.

The pressure wave can carry a frequency to have a therapeutic benefit. While not to be bound by a theory, different frequency bands can have different therapeutic benefits. The frequency can restore natural vessel function. The frequency can promote conditions for the vessel to heal itself. The frequency can provide for calcium disruption by applying a shearing force. The frequency can provide for controlled fracturing. The frequency can be utilized for various lesion formations and depths. The frequency can be effective in calcified, mixed-morphology, and fibrotic lesions. The frequency can reach deep locations within the vessel. The frequency can change vessel compliance through controlled pressure wave assisted expansion of the balloon 100.

The pressure wave can have an ultrasonic frequency. The pressure wave can have a frequency greater than 20000 Hz (20 kHz). The pressure wave can have a ultrasonic frequency of 20000 Hz (20 kHz), 21000 Hz (21 kHz), 22000 Hz (22 kHz), 23000 Hz (23 kHz), 24000 Hz (24 kHz), 25000 Hz (25 kHz), 26000 Hz (26 kHz), 27000 Hz (27 kHz), 28000 Hz (28 kHz), 29000 Hz (29 kHz), 30000 Hz (30 kHz), 31000 Hz (31 kHz), 32000 Hz (32 kHz), 33000 Hz (33 kHz), 34000 Hz (34 kHz), 35000 Hz (35 kHz), 36000 Hz (36 kHz), 37000 Hz (37 kHz), 38000 Hz (38 kHz), 39000 Hz (39 kHz), 40000 Hz (40 kHz), 41000 Hz (41 kHz), 42000 Hz (42 kHz), 43000 Hz (43 kHz), 44000 Hz (44 kHz), 45000 Hz (45 kHz), 46000 Hz (46 k Hz), 47000 Hz (47 kHz), between 20 kHz and 50 kHz, or any range of two of the foregoing values. The pressure wave can have a ultrasonic frequency of 20000 Hz (20 kHz), 25000 Hz (25 kHz), 30000 Hz (30 kHz), 35000 (35 kHz), 40000 Hz (40 kHz), 44000 Hz (44 kHz), 50000 Hz (50 kHz), 55000 Hz (55 kHz), 60000 Hz (60 kHz), 65000 Hz (65 kHz), 70000 Hz (70 kHz), 75000 Hz (75 kHz), 80000 Hz (80 kHz), 85000 Hz (85 kHz), 90000 Hz (90 kHz), 95000 Hz (95 kHz), 100000 Hz (100 kHz), 105000 Hz (105 kHz), 110000 Hz (110 kHz), 115000 Hz (115 kHz), 120000 Hz (120 kHz), 125000 Hz (125 kHz), 130000 Hz (130 kHz), 135000 (135 kHz), 140000 Hz (140 kHz), 145000 Hz (145 kHz), 150000 Hz (150 kHz), 155000 Hz (155 kHz), 160000 Hz (160 kHz), 165000 Hz (165 kHz), 170000 Hz (170 kHz), 175000 Hz (175 kHz), 180000 Hz (180 kHz), 185000 Hz (185 kHz), 190000 Hz (190 kHz), 195000 Hz (195 kHz), 200000 Hz (200 kHz), 205000 Hz (205 kHz), 210000 Hz (210 kHz), 215000 Hz (215 kHz), 220000 Hz (220 kHz), 225000 Hz (225 kHz), 230000 Hz (230 kHz), 235000 (235 kHz), 240000 Hz (240 kHz), 245000 Hz (245 kHz), 250000 Hz (250 kHz), between 20 kHz and 100 kHz, between 40 kHz and 100 kHz, up to 120 kHz, up to 200 kHz, up to 250 kHz, or any range of two of the foregoing values. While not to be bound by a theory, the ultrasonic frequency of the pressure wave can facilitate calcium fracturing. The ultrasonic frequency can cause shearing of the calcium sheet. The ultrasonic frequency can allow for calcium fragmentation. The ultrasonic frequency can yield better patient outcomes and advantages as described herein. The ultrasonic frequency can be applied directly to the calcium when the raised features 300 are in direct contact with the calcium. The ultrasonic frequency can be applied directly to the calcium when the lobes 120 are in direct contact with the calcium. The pressure wave can have an ultrasonic band between 20 kHz and 40 kHz. The ultrasonic band can carry most of the energy of the pressure wave. The pressure wave can have a smaller ultrasonic band above 40 kHz. The pressure wave can have a defined frequency induced within the pressure wave. The pressure wave can transmit a frequency in the ultrasonic spectrum.

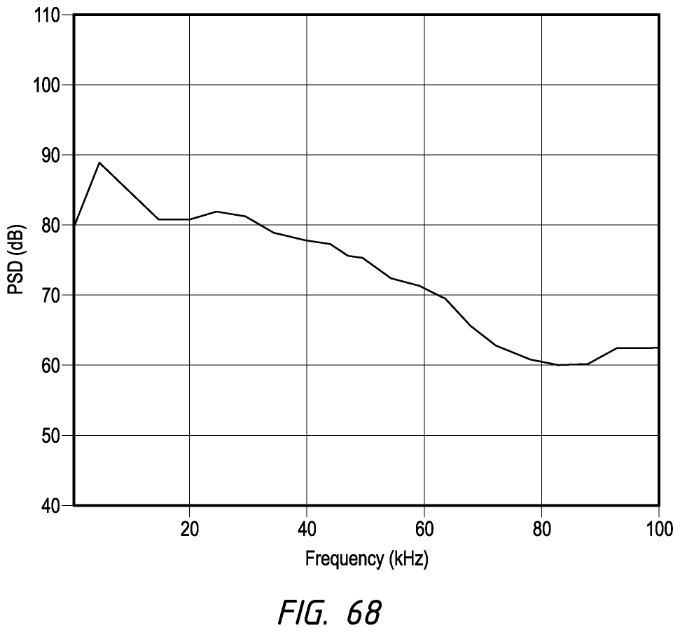

FIG. 68 illustrates a frequency range captured during testing of the system 1100. The graph illustrates the Power Spectral Density (PSD) in decibels (dB) per unit frequency (kHz). The graph represents how signal energy is distributed across frequencies. The graph demonstrates frequencies that were generated by the system 1100 in the acoustic range (e.g., under 20 kHz). The graph demonstrates frequencies that were generated by the system 1100 in the ultrasonic range (e.g., above 20 kHz). The graph demonstrates frequencies that were generated by the system 1100 in the ultrasonic range up to 100 kHz. The frequencies can be greater than 100 kHz as described herein (e.g., up to 125 kHz, up to 200 kHz).

The pressure wave can have a sonic frequency. The pressure wave can have a frequency equal to or greater than 1 kHz. The pressure wave can have sonic frequency of less than 20000 Hz (20 kHz). The pressure wave can have sonic frequency of 20 Hz, 100 Hz, 200 Hz, 300 Hz, 400 Hz, 500 Hz, 1000 Hz (1 kHz), 5000 Hz (5 kHz), 10000 Hz (10 kHz), 15000 Hz (15 kHz), 20000 Hz (20 kHz), between 1 kHz and 10 kHz, between 10 kHz and 20 kHz, between 1 kHz and 20 kHz, below 20 kHz, or any range of two of the foregoing values. While not to be bound by a theory, the sonic frequency can have a therapeutic effect. The sonic waves can cause can deliver energy. The sonic waves can cause calcium to fracture. The sonic waves can travel through softer tissue. The pressure can have a sub-ultrasonic band. The pressure wave can have an acoustic band.

The pressure wave can have a frequency that emits from the lobes 120 of the balloon 100. The pressure wave can have a frequency that emits from the strip 200. The pressure wave can have a frequency that emits from the raised features 300. The pressure wave can have a frequency that is detected when facing the serrated edge of the strips 200. The pressure wave can have a frequency that is detected when facing away from the serrated edge of the strips. The pressure wave can have a frequency that is detected at the proximal end of the balloon 100 with the strips 200 and raised features 300. The pressure wave can have a frequency that is detected at a midpoint the balloon 100 with the strips 200 and raised features 300. The pressure wave can have a frequency that is detected proximal of the balloon 100 with the strips 200 and raised features 300. The pressure wave can have a frequency that is detected at the distal end of the balloon 100 with the strips 200 and raised features 300. The frequency detected can be any range described herein.

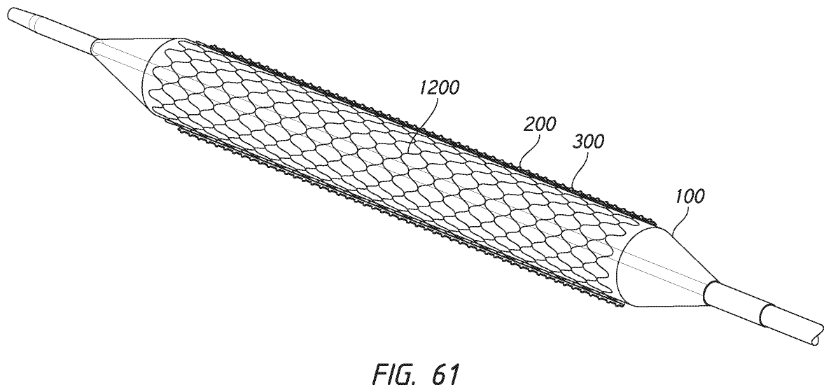

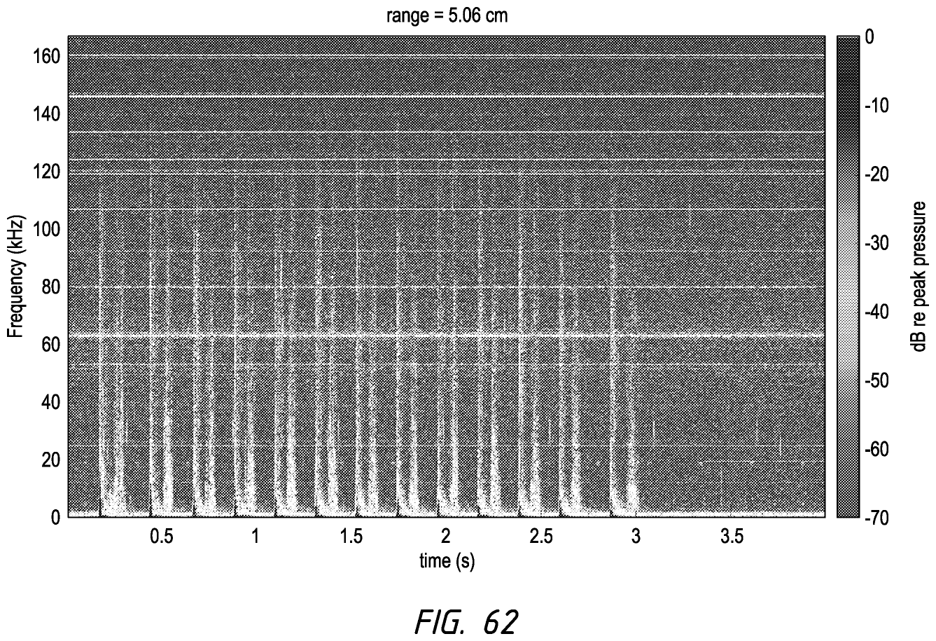

The pressure waves can carry one or more frequencies. The pressure waves can include infrasonic frequencies The pressure waves can include acoustic frequencies. The pressure waves can include ultrasonic frequencies. FIG. 62 illustrate the ultrasonic propagation in the radial scan data. The balloon 100 can radiate ultrasonic energy. The frequency is above 20 kHz. The frequency up to about 120 kHz is present. There can be acoustic propagation of sonic frequencies up to 20 kHz. There can be acoustic propagation of ultrasonic frequencies of 20 kHz to 120 kHz There can be acoustic propagation of ultrasonic frequencies up to 500 kHz. The figure illustrates that the balloon definitively radiates ultrasonic energy. The radial scan data was further analyzed using short-time Fourier transforms. The noise was sufficiently far below the ultrasonic signal. The radial scan compared mean maximum pressure to distance from the balloon. The mean maximum pressure can be about 61 Pa close to the balloon, e.g., less than 5 mm. The mean maximum pressure can be about 41 Pa about 10 mm from the balloon 100. The mean maximum pressure can be about 32 Pa about 25 mm from the balloon 100. The mean maximum pressure can be about 23 Pa about 50 mm from the balloon 100. The mean maximum pressure can be about 9-11 Pa about 100 mm from the balloon 100. The mean maximum pressure can be less than 10 Pa greater than 100 mm from the balloon 100. The data can be on a log-log plot and spectrograms can be calculated from a selection of data in the far field. FIG. 62 is a spectrogram for the data. The balloon 100 was subjected to pressure waves and the spectrogram illustrates the frequency outside of the ballon 100 over time. The frequency was in the ultrasonic range, greater than 20 kHz. The direct wave from the balloon can be the first detected peak, about 120 kHz to 140 kHz. The peak pressure was measured along the first peak. The dB of the peak pressure was about 40 dB above tonal noise. The acoustic energy above 20 kHz is present. The acoustic energy above 120 kHz is present.

The system 1100 can have additional parameters as described herein. In some embodiments, one or more of the parameters can be controlled by the user such as the motor frequency and the spring compression described herein. In some embodiments, one or more of the parameters are not controlled by the user such as parameters predetermined by the system itself. In some embodiments, one or more of the parameters relate to the pressure wave generated by the system 1100.

The system 1100 can deflect a volume of fluid with the deflection of the membrane 884. The system 1100 can move fluid. The system 1100 can create a pressure difference. The system 1100 can convert mechanical energy into hydraulic energy. The system 1100 can move fluid from one place to another. The pressure wave can include a fluid wave. The fluid wave can move from the reservoir 854 to the balloon 100 along the length of the system 1100. The system 1100 can include a volume displacement per deflection. The cam hammer 848 to the piston 878 to the membrane 884 distance can be related to two distinct variables. The first variable can be the pressure against the wall on the catheter side of the membrane. The second variable can be the distance that the piston 878 can extend the membrane 884 outwardly into the reservoir 854. The limit of travel of the piston 878 can be set by the travel of the cam hammer 848.

The volume displacement at the membrane 884 can be 100 mm³, 150 mm³, 200 mm³, 250 mm³, 300 mm³, 350 mm³, 400 mm³, 450 mm³, 500 mm³, 550 mm³, 600 mm³, 650 mm³, 700 mm³, 750 mm³, 400 mm³ and 500 mm³, between 450 mm³ and 550 mm³, about 471 mm³, 0.1 cc, 0.15 cc, 0.2 cc, 0.25 cc, 0.3 cc, 0.35 cc, 0.4 cc, 0.45 cc, 0.5 cc, 0.55 cc, 0.6 cc, 0.65 cc, 0.7 cc, 0.75 cc, or any range of two of the foregoing values.

The balloon 100 can experience a change in diameter from the pressure wave. The change in diameter can be 0.1 mm, 0.2 mm, 0.3 mm, 0.4 mm, 0.5 mm. 0.6 mm, 0.7 mm, 0.8 mm, 0.9 mm, 1 mm, between 0 mm and 0.3 mm, between 0 mm and 0. 5 mm, or any range of two of the foregoing values. The balloon 100 can experience a volume change from the pressure wave. The volume displacement in the balloon 100 can be 0.1 mm³, 0.2 mm³, 0.3 mm³, 0.4 mm³, 0.5 mm³, 0.6 mm³, 0.7 mm³, 0.8 mm³, 0.9 mm³, 1 mm³, 2 mm³, 3 mm³, 4 mm³, 5 mm³, 6 mm³, 7 mm³, 8 mm³, 9 mm³, 10 mm³, 11 mm³, 12 mm³, 13 mm³, 14 mm³, 15 mm³, 20 mm³, 25 mm³, 30 mm³, 35 mm³, between 0 mm³ and 1 mm³, between 0 mm³ and 10 mm³, 0.001 cc, 0.005 cc, 0.01 cc, 0.02 cc, 0.03 cc, 0.035 cc, or any range of two of the foregoing values. The volume of the balloon 100 can change minimally. The balloon 100 can be in contact with the vessel at the base pressure. The balloon 100 can be in contact with the vessel at the peak positive pressure. The balloon 100 can be constrained by the vessel during the pressure wave. The strip 200 with the raised features 300 can experience strip motion in microns. The strip 200 and raised features 300 motion can be 50 microns, 100 microns, 150 microns, 200 microns, 250 microns, 300 microns, 350 microns, 400 microns, between 50 micron and 150 microns, under 350 microns, or any range of two of the foregoing values.

While not to be limited by a theory, the membrane 884 can rapidly displace the fluid to create pressure waves that propagate mechanical energy through the fluid. The forward movement of the membrane 884 can push on the fluid creating a region of higher pressure. The backward motion of the membrane can create a lower pressure region. The alternating compression and rarefaction can travel through the fluid of the system 1100. The pressure wave can create a fluid wave that imparts a wave motion on the strip. There can be a physical displacement of fluid to expand the diameter of the balloon 100. There can be a physical displacement of fluid to cause micromotion of the strips 200 with raised features. The resulting micromotion motion of the strip can be caused by pressure wave. There can be a combined effect of the pressure wave and the fluid motion. The pressure disturbance and the motion of the fluid can have a combined effect. The strip 200 with the raised features 300 can move radially during the pressure wave. The raised features 300 can be in contact with the vessel wall at the base pressure. The pressure wave can cause micromotion of the raised features 300. The pressure wave can causes the raised features 300 to zipper into and out of the plaque on the order of microns.

The system 1100 can have parameters related to volume displacement. In the illustrated embodiment, the accuracy of the travel of the cam hammer 848 can be +/−0.1 mm which corresponds to 3 mm maximum travel of the cam hammer 848. In the illustrated embodiment, at the maximum movement of the cam hammer 848, the maximum displacement of the membrane 884 can be 1.5 mm. In the illustrated embodiment, this displacement of the membrane 884 can correspond to a volume displaced of about 471 mm³ or about 0.47 cc. As the pressure in the reservoir 854 is increased, there can be an equilibrium state that is reached by the potential energy of the spring 846 pushing forward and the back pressure due to the indeflator pressure on the catheter side of the membrane 884. As the spring 846 is compressed, the potential energy of the generator 800 is increased. The equilibrium state can shift towards a higher pressure state based on the spring compression. Independent of these changes in the spring compression and the indeflator pressure, the minimum volume displacement in the balloon 100 can be 0 cc and the maximum volume displacement is 0.5 cc. In the illustrated embodiment, the area of the piston 878 can be 314 mm². In the illustrated embodiment, the displacement of the piston 878 can be 1.5 mm.

The system 1100 can include an accuracy of the deflection volume. In some embodiments, the change in volume can vary with the balloon size. The back pressure can stop the piston 878 from moving forward. Given a known balloon size of the balloon 100, a known pressure set at the indeflator, and a known spring tension of the spring 846, the deflection of the membrane 884 can be within a tolerance of 1-10% repeatable hit-to-hit of the piston 878 to the membrane 884. In some embodiments, the main contributing factor to loss in repeatability is over time can be the degradation of the cam hammer 848 and any wear in the inner diameter of the hammer bearing block 852. In some embodiments, combined this potential wear could limit transmission of energy from the spring 846 to the piston 878 and the membrane 884. During testing performed at highest frequency and highest intensity over 10,000, minimal wear has been observed with less than a 20-30% loss in membrane deflection. The spring force tolerance range can be 1% to 10%, such as 5%.

In some embodiments, it can be advantageous for the volume displacement to be within the range described herein. While not to be limited by a theory, the volume displacement of the system 1100 described herein can allow for the pressure wave generation within the fluid of the system 1100. The balloon 100 can be filled with an incompressible fluid. The balloon 100 can be contact with the vessel wall. The pressure wave can be generate by rapidly compressing and decompressing the fluid of the system 1100. The pressure wave can be generated by the mechanical deflection of the membrane 848 by the cam hammer 848 driven by the motor 830. The membrane 8484 can be mechanically moved to create alternating compressions and rarefactions in the fluid of the system 1100. The membrane 8484 can be mechanically moved in a controlled manner. The membrane 848 can be mechanically moved to accurately deliver the same pressure wave repeatedly.

The system 1100 can include a rise time parameter. The rise time can be the time delay per unit of pressure. The rise time in terms of time and pressure can be 1 nanosecond per pascal, 10 nanoseconds per pascal, 50 nanoseconds per pascal, 75 nanoseconds per pascal, 100 nanoseconds per pascal, 125 nanoseconds per pascal, 150 nanoseconds per pascal, 175 nanoseconds per pascal, 200 nanoseconds per pascal, 225 nanoseconds per pascal, 250 nanoseconds per pascal, 275 nanoseconds per pascal, 300 nanoseconds per pascal, 325 nanoseconds per pascal, 350 nanoseconds per pascal, 375 nanoseconds per pascal, 400 nanoseconds per pascal, 425 nanoseconds per pascal, 450 nanoseconds per pascal, 475 nanoseconds per pascal, 500 nanoseconds per pascal, 750 nanoseconds per pascal, 1 microsecond per pascal, between 1 nanosecond per pascal and 500 nanoseconds per pascal, between 100 nanoseconds per pascal and 300 nanoseconds per pascal, or any range of two of the foregoing values. The rise time can be 10 microseconds, 15 microseconds, 20 microseconds, 25 microseconds, 30 microseconds, 35 microseconds, 40 microseconds, 45 microseconds, 50 microseconds, between 10 microseconds and 50 microseconds, or any range of two of the foregoing values. The rise time can be about 30 microseconds. The rise time can be about 40 microseconds. The rise time can have a lower limit of 0.5 microseconds, 1 microseconds, 2 microseconds, 3 microseconds, 4 microseconds, 5 microseconds, 6 microseconds, 7 microseconds, 8 microseconds, 9 microseconds, 10 microseconds, 15 microseconds, 20 microseconds, 30 microseconds, or any range of two of the foregoing values. The rise time can have an upper limit of 30 microseconds, 35 microseconds, 40 microseconds, 45 microseconds, 50 microseconds, 55 microseconds, 60 microseconds, 70 microseconds, 75 microseconds, 80 microseconds, 85 microseconds, 90 microseconds, or any range of two of the foregoing values. The rise time can have a range of any two values described herein. The rise time can be 33.5 microseconds.

The rise time can include a portion of linear rise. The linear rise of the rise time can be 0 microseconds, 2.5 microseconds, 5 microseconds, 7.5 microseconds, 10 microseconds, 12.5 microseconds, 15 microseconds, 17.5 microseconds, 20 microseconds, 22.5 microseconds, 25 microseconds, or any range of two of the foregoing values. The linear rise of the rise time can be less than 15 microseconds. The linear rise of the rise time can be about 13 microseconds.

While not to be limited by a theory, the rise time can correlate with the frequency of the pressure wave. The rise time can correlate with the acoustic frequency of the pressure wave. The rise time can correlate with the ultrasonic frequency of the pressure wave. The rise time and the frequency carried by the pressure wave can be inversely correlated. The higher frequency, ultrasonic pressure waves can have a shorter rise time. In some embodiments, the pressure wave can comprise a frequency greater than 20 kHz. The pressure wave can include a frequency signature including at least ultrasonic frequency.

The system 1100 can include a pulse width parameter. The pulse width can be defined as the full width at half maximum (FWHM). The pulse width can be 0.5 microseconds, 1 microseconds, 10 microseconds, 50 microseconds, 100 microseconds, 150 microseconds, 200 microseconds, 250 microseconds, 300 microseconds, 350 microseconds, 400 microseconds, 450 microseconds, 500 microseconds, 550 microseconds, 600 microseconds, 650 microseconds, 700 microseconds, 750 microseconds, 800 microseconds, 850 microseconds, 900 microseconds, 950 microseconds, 1 millisecond, 1.5 milliseconds, 2 milliseconds, 2.5 milliseconds, 3 milliseconds, between 0.5 microseconds and 3 milliseconds, between 300 microseconds and 500 microsecond, between 100 microsecond and 1 milliseconds, or any range of two of the foregoing values.

The system 1100 can include a peak pressure at the reservoir 854 of the generator 800. The peak pressure at the reservoir 854 can be 1000 PSI, 1100 PSI, 1200 PSI, 1300 PSI, 1400 PSI, 1500 PSI, 1600 PSI, 1700 PSI, 1800 PSI, 1900 PSI, 2000 PSI, between 1000 PSI and 2000 PSI, or any range of two of the foregoing values. The peak pressure at the reservoir 854 can be about 1500 PSI. The peak pressure at the reservoir 854 can be about 1750 PSI. The peak pressure at the reservoir 854 can be about 1100 PSI. The peak pressure at the reservoir 854 can have a lower limit of 500 PSI, 600 PSI, 700 PSI, 800 PSI, 900 PSI, 1000 PSI, 1100 PSI, 1200 PSI, 1300 PSI, 1400 PSI, 1500, or any range of two of the foregoing values. The peak pressure at the reservoir 854 can have an upper limit of 1500 PSI, 1600 PSI, 1700 PSI, 1800 PSI, 1900 PSI, 2000 PSI, 2100 PSI, 2200 PSI, 2300 PSI, 2500 PSI, or any range of two of the foregoing values. The peak pressure at the reservoir 854 can have a range of any two values described herein.

The system 1100 can include a waveform shape parameter. The waveform can have a rapid rise at the front of the wave. This rapid rise can be due to the percussive impact energy traveling to the balloon 100. The back side of the waveform can be broader and dampened with a decaying shape. The waveform shape can be sinusoidal. The waveform shape can be a smooth periodic oscillation. The waveform shape can have a repeating pattern. The waveform shape can be damped sinusoid wave. The waveform can be a sinusoid oscillation with an amplitude that decreases over time. The waveform shape can have a decaying envelop. The waveform shape can become flatter over time. The waveform shape can be a decaying sinusoid wave. The waveform shape can have a decay constant. The waveform shape can have a varying frequency sinusoid wave. The waveform shape can have a frequency-modulated signal. The waveform shape can have an instantaneous frequency that changes over time. The waveform shape can have a non-constant frequency. The waveform shape can be a varying frequency and amplitude sinusoid wave. The waveform shape can have a non-stationary signal. The waveform shape can have a modulated signal.

The system 1100 can include pulses per cycle parameter. The cycle can be defined as the length of time per treatment of a single treatment zone. The cycle can be defined as a length of time that the generator 800 is activated. The cycle can be the length of the procedure. A range of pulses may be adequate to produce a positive remodeling effect at a treatment zone. The number of pulses per cycle can include 1 pulse per cycle, 2 pulses per cycle, 3 pulses per cycle, 4 pulses per cycle, 5 pulses per cycle, 6 pulses per cycle, 7 pulses per cycle, 8 pulses per cycle, 9 pulses per cycle, 10 pulses per cycle, 20 pulses per cycle, 30 pulses per cycle, 40 pulses per cycle, 50 pulses per cycle, 60 pulses per cycle, 70 pulses per cycle, 80 pulses per cycle, 90 pulses per cycle 100 pulses per cycle, or any range of two of the foregoing values. In some embodiments, there is no upper limit of pulses that the generator 800 would be bound to other than the practical limit of time during a procedure. Unlike other systems, such as those that require an arc generator to create shockwaves, the generator 800 may not require a pause to reset. There may be a practical limit of the procedure, such as 10 minutes. Therefore, the upper limit of pulses per cycle can be 1800 pulses per cycle. The system 1100 can continuously produce pulses until powered off. The system 1100 can continuously produce pulses until paused by the user. The system 1100 can continuously produce pulses at a single treatment site. The system 1100 can continuously produce pulses during the cycle. Unlike other systems, such as those that require an arc generator to create shockwaves, the generator 800 may not require a pause between cycles. Therefore, the single treatment site can be treated with one cycle. In some methods, the single treatment site can be treated with more than one cycle.

The system 1100 can include number of cycles parameter. The number of cycles can be defined as the number of treatment zones. A range of cycles may be adequate to produce a positive remodeling effect in multiple treatment zones in more than one location throughout the body. The number of cycles can be defined by the number of treatment zones before removal from the body. The number of cycles can be 1 cycle, 2 cycles, 3 cycles, 4 cycles, 5 cycles, 5 cycles, 6 cycles, 7 cycles, 8 cycles, 9 cycles, 10 cycles, 11 cycles, 12 cycles, 13 cycles, 14 cycles, 15 cycles, or any range of two of the foregoing values. The balloon 100 can be deflated to be moved to another treatment site. In some methods, the system 1100 does not produce pressure waves as the balloon 100 is moved to another treatment site. The movement of the balloon 100 to another treatment site can be a pause between cycles. In some embodiments, there is no upper limit of cycles that the generator 800 would be bound to other than the practical limit of time during a procedure. There may be a practical limit of the procedure, such as 10 treatment sites. Therefore, the upper limit of can be 18,000 pulses in total over 10 treatment sites.

While not to be limited by a theory, the system 1100 can produce results with a single pressure wave. The pressure wave can crack calcium in a single cycle. The pressure wave can create longitudinal cracks with a single pressure wave. The calcium can yield with a few pressure waves such as 1 pressure wave, 2 pressure waves, 3 pressure waves, 4 pressure waves, 5 pressure waves, less than 5 pressure waves, less than 10 pressure waves, or any range of two of the foregoing values. In contrast, other lithotripsy systems may require 10 pulses per cycle, and between 8 and 10 cycles. Other lithotripsy systems may also require a time period between cycles in order to dissipate energy and/or clear bubbles. While not to be limited by a theory, the strips 200 with the raised features 300 can significantly reduce the pressure waves per cycle. The strips 200 with the raised features 300 can accelerate the time to yield. The strips 200 with the raised features 300 can facilitate energy transmission, thereby reducing the pulses per cycle.

The system 1100 can include a motor frequency parameter of the motor 830. The motor 830 can spin one time per one hit on the membrane 884. The motor 830 can spin one time per two hits on the membrane 884. The motor 830 can spin one time per three hits on the membrane 884. The motor 830 can spin one time per four hits on the membrane 884. The motor 830 can spin one time per five hits on the membrane 884. The motor 830 can spin one time per six hits on the membrane 884. In the illustrated embodiment, each rotation of the motor shaft 831 of the motor 830 can cause three hits of the membrane 884 by the piston 878. Therefore, in the illustrated embodiment, when we are hitting the membrane 884 at 1 Hz the motor 830 is rotating at 0.3 Hz. Therefore, in the illustrated embodiment, when we are hitting the membrane at 3 Hz the motor 830 is rotating at a frequency of 1 Hz.

The motor 830 can have parameters to enable the generation of pressure waves described herein. In some embodiments, the motor 830 can be a synchronous motor. In contrast to synchronous motors, stepper motors can offer high torque and precision motion control but typically run slower. Stepper motors typically require complicated drivers. The drivers for a stepper motor provide precise pulses of power to certain poles within the motor at a pre-determined rate to make the motor turn. Stepper motors can break rotation into discrete steps. The system 1100 can use a brushless DC motor with an integrated controller. The system 1100 can allow for the application of direct power to the motor 830 to drive the motor 830. In the illustrated embodiment, to produce the torque needed for driving the cam hammer 848, the system 1100 can use a geared 12000 RPM motor, with a planetary gear box to gear down the RPM at a 138:1 ratio. This significantly increases the torque and allows for similar precision motor control.

The system 1100 can include a strike frequency parameter. The striking frequency can set by the operator by the speed control 810. The striking frequency can be directly correlated to the pulse frequency which is equivalent to 1 Hz to 3 Hz.

The system 1100 can include a striking force. The system 1100 can include a striking force of the cam hammer 848 to the piston 878. The system 1100 can include a striking force of the piston 878 to the membrane 884. The system 1100 can include a spring force to achieve the pressure rise. The spring force can be 100 lb/in, 125 lb/in, 150 lb/in, 175 lb/in, 200 lb/in, 225 lb/in, 250 lb/in, 275 lb/in, 300 lb/in, 325 lb/in, 350 lb/in, 375 lb/in, 400 lb/in, between 200 lb/in and 400 lb/in, or any range of two of the foregoing values. The pressure rise can be 3 ATM to 7 ATM. The system can have an equilibrium pressure between 19 lb and 50 lb.

The system 1100 can include a striking accuracy. The piston 878 can be constrained within the generator 800. The cam hammer 848 can be constrained within components of the generator 800. The inner diameter tolerance can be +/−0.5 mm where the cam hammer 848 hits the piston 878. The inner diameter tolerance can be +/−0.5 mm where the piston 878 hits the membrane 884. Due distance of travel, the angle of piston misalignment can be negligible. The striking accuracy of the cam hammer 848 to the piston 878 can be +/−0.25 mm. The striking accuracy of the piston 878 to the membrane 884 can be +/−0.5 mm.

The system 1100 can have a pulse duration parameter. In some embodiments, the pulse duration can be 1 microsecond, 2 microseconds, 3 microseconds, 4 microseconds, 5 microseconds, 10 microsecond, 20 microseconds, 30 microseconds, 40 microseconds, 50 microseconds, 100 microseconds, 500 microseconds (0.5 milliseconds), 600 microseconds, 600 microseconds (0.6 milliseconds), 700 microseconds (0.7 milliseconds), 800 microseconds (0.8 milliseconds), 900 microseconds (0.9 milliseconds), 1 millisecond, 1.1 milliseconds, 1.2 milliseconds, 1.3 milliseconds, 1.4 milliseconds, 1.5 milliseconds, 1.6 milliseconds, 1.7 milliseconds, 1.8 milliseconds, 1.9 milliseconds, 2 milliseconds, 3 milliseconds, 4 milliseconds, 5 milliseconds, between 1 microsecond and 5 milliseconds, between 0.5 milliseconds and 1.5 milliseconds, between 0.8 milliseconds and 1.1 milliseconds, or any range of two of the foregoing values. The pulse shape and/or the duration can be different between the generator 800 and the balloon 100. The different modes of transmissions (plane wave, bulk wave, etc.) can exhibit different speeds and different attenuations and thus by the time all these modes arrive at the balloon 100 the net sum of them can result in a very different wave shape and duration.

The system 1100 can return to the base pressure due to back pressure within the system 1100. The peak positive pressure can be dissipated through the tissue and distally as the pressure waves travel. The system 1100 can return to the base pressure within the reservoir 854. The system 1100 can return to the base pressure within the catheter 600. The system 1100 can return to the base pressure within the balloon 100. The system 1100 can return to the base pressure to reset the position of the membrane 884. The system 1100 can return to base pressure at the speed of propagation of the pressure wave. The system 1100 can return to base pressure at the speed of sound. The system 1100 can return to base pressure as the pressure wave passes.

The system 1100 can have a threshold pressure of the balloon 100. The threshold pressure can be the pressure that the balloon should not be inflated above. The threshold pressure can be the rated burst pressure of the balloon 100. The balloon 100 can be inflated up to the burst limits of the balloon 100. The balloon 100 can be inflated above tested burst limits in some embodiments.

The system 1100 can have a parameter related to the volume of the balloon 100. The total fluid space internal to the balloon 100 can be a calculated volumetric combination of a cylinder with two conical ends. The volume of the balloon 100 can be a function of the diameter of the balloon and the length of the balloon 100. The diameter of the balloon can be 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, or any range of two of the foregoing values. The length of the balloon can be 20 mm, 40 mm, 60 mm, 80 mm, 100 mm, 120 mm, or any range of two of the foregoing values. In some embodiments, the diameter of the balloon changes 0.1 mm, 0.2 mm, 0.3 mm, 0.4 mm, 0.5 mm, 0.6 mm, 0.7 mm, or any range of two of the foregoing values. In some embodiments, the diameter of the balloon changes 0%, 1%, 2%, 3%, 4%, 5%, 6%, 7%, 8%, 9%, 10%, 11%, 12%, 13%, 14%, 15%, under 5%, under 10%, under 15%, or any range of two of the foregoing values. The balloon volume can typically range from 30 mm³ to 3,400 mm³. The balloon volume can typically range from 15 mm³ to 21,000 mm³. In some embodiments, the volume of the balloon changes minimally. In some embodiments, the volume change of the balloon 100 for balloon can be 0.2 mm³, 0.3 mm³, 0.4 mm³, 0.5 mm³, 0.6 mm³, 0.7 mm³, 0.8 mm³, 0.9 mm³, 1 mm³, 2 mm³, 3 mm³, 4 mm³, 5 mm³, 6 mm³, 7 mm³, 8 mm³, 9 mm³, 10 mm³, 11 mm³, 12 mm³, 13 mm³, 14 mm³, 15 mm³, 16 mm³, or any range of two of the foregoing values. In some embodiments, the volume change of the balloon 100 for balloon greater than 120 mm in length can be 1 mm³, 8 mm³, 9 mm³, 10 mm³, 11 mm³, 12 mm³, 13 mm³, 14 mm³, 15 mm³, 20 mm³, 25 mm³, 30 mm³, 35 mm³, or any range of two of the foregoing values. The volumes of the balloon can change minimally above 2 ATM.

The system 1100 can have a parameter related to strip motion. The strip 200 can move 25 microns, 50 microns, 75 microns, 100 microns, 125 microns, 150 microns, 175 microns, 200 microns, 250 microns, 275 microns, 300 microns, 325 microns, 350 microns, or any range of two of the foregoing values. The lower limit of movement of the strip 200 can be 0 microns, 5 microns, 10 microns, 15 microns, 20 microns, 25 microns, 30 microns, 40 microns, 50 microns, 60 microns, 70 microns, 80 microns, 90 microns, 100 microns, or any range of two of the foregoing values. The upper limit of movement of the strip 200 can be 50 microns, 100 microns 150 microns, 200 microns, 250 microns, 300 microns, 350 microns, 400 microns, 450 microns, 500 microns, or any range of two of the foregoing values.

The system 1100 can have a parameter related to the time of the pressure wave to travel along the length of the catheter 600. The catheter 600 can have a length of about 1100 mm. The time for the pressure wave to travel along this catheter length can be 0.72 milliseconds. The catheter 600 can have a length of about 1500 mm. The time for the pressure wave to travel along this catheter length can be 0.98 milliseconds.

The system 1100 can have a parameter related to the time of the pressure wave to travel along the length of the balloon 100. The balloon can have a length of about 20 mm. The time for the pressure wave to travel along this balloon length can be 0.013 milliseconds. The balloon can have a length of about 40 mm. The time for the pressure wave to travel along this balloon length can be 0.026 milliseconds. The balloon can have a length of about 80 mm. The time for the pressure wave to travel along this balloon length can be 0.052 milliseconds. The balloon can have a length of about 120 mm. The time for the pressure wave to travel along this balloon length can be 0.078 milliseconds.

The pressure wave can travel at the speed of sound through the various mediums. The pressure wave can travel between about 1480 m/s and 1530 m/s through the liquid medium used to fill the system 1100. The pressure wave can travel about 1540 m/s through soft tissue of the vasculature. The pressure wave can travel between 5850 m/s and 5920 m/s through the strips 200 with the raised features 300. The pressure wave can travel at about 1526 m/s at body temperature (40 C). The pressure wave can travel at about 1580 m/s to 1630 m/s when traveling through 50/50 contrast at body temperature (40 C).

The pressure wave can have minimal dissipation from the generator 800 to the balloon 100. The energy loss between the reservoir 854 and the balloon 100 can be realized during pressure impulse transit. The energy loss can be due to the strain response of all components including the reservoir 854, the catheter 600, and the balloon 100 subjected to the impulse pressure.

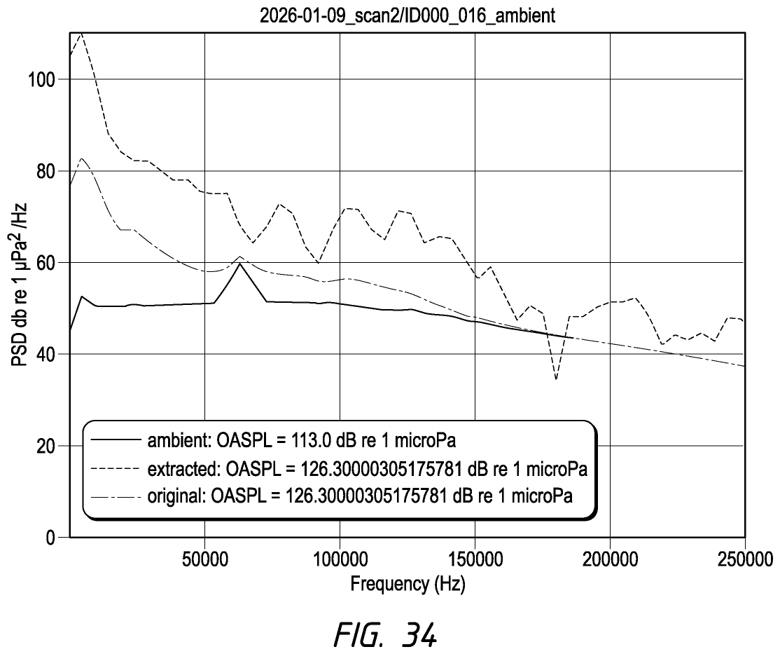

FIG. 34 illustrates the extracted signal of the pressure wave in some embodiments. The signal was measured 400 microseconds around the peak of the pressure wave. In some embodiments, because the extracted level is greater than the measurement peak noise at 62.5 kHz, bandpass filtering may not be needed. The figures illustrates the ambient signal in blue, the extracted signal in black, and the original signal in red. The signal has a spike around 2 kHz. The signal can have a frequency in the ultrasonic range greater than 20 kHz into the 150 kHz range. The signal can have a frequency in the ultrasonic range greater than 20 kHz.

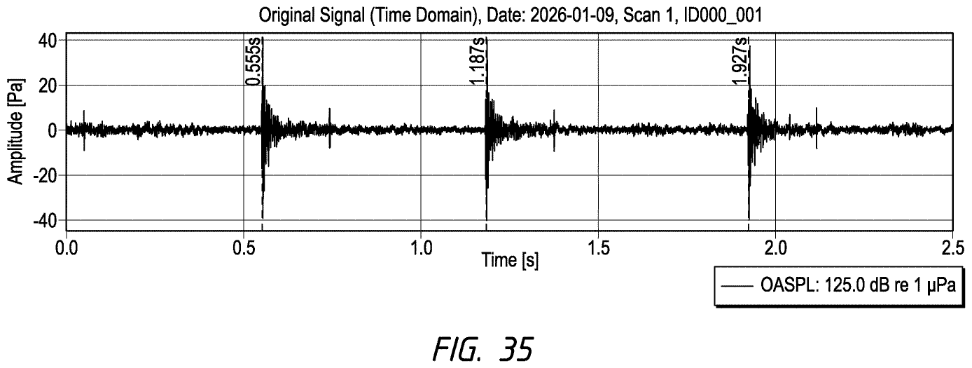

FIG. 35 illustrates the time domain of the pressure wave in some embodiments. The signal was measured in 2.5 seconds at a 10 MHz sampling rate. The data was extracted 0.5 ms before and 1.5 ms after peak in each event. The frequency of the pressure wave is approximately 2.5 Hz. The amplitude of the pressure wave is approximately 80 Pa. In some embodiments, the max pressure is about 40 Pa. In some embodiments, the peak pressure can be 38+/−20 Pa. The 10 db down frequencies can be 38+/−10 kHz. The rise can be between 240 ns/Pa and 270 ns/Pa. The fastest rise time can be 239+/−60 ns/PA The data can be developed in power spectral density plots.

While not to be limited by a theory, the raised features 300 can be particularly well suited for energy transmission. The raised features 300 can be longitudinally oriented having a longer length than width. The radially outward surface 304 of the raised features 300 can be longitudinally oriented having a longer length than width. The raised features 300 can direct the energy from the pressure wave longitudinally along the serrated lines. The raised features 300 can direct the energy from the pressure wave to facilitate crack propagation along longitudinal lines. The raised features 300 can direct the energy from the pressure wave to speed up crack propagation, in some instances by 10×. The raised features 300 can concentrate the energy from the strip 200 to smaller surface areas of the radially outward surfaces 304. The raised features 300 can amplify the pressure wave as the wave propagates. The raised features 300 can have surface imperfections. The pressure waves can be sensitive to surface imperfections. The surface imperfections of the raised features 300 can significantly magnify shear stress imparted on the calcium. While not to be limited by a theory, the raised features 300 can act as stress concentrators. The raised features 300 can be in direct contact with the calcium. The raised features 300 can create areas where shear stress is higher. The raised features can provide alternating shear stress by the rolling contact of the raised features 300 with the calcium. The raised features 300 can include a surface roughness which can increase shear stresses. The surface roughness can be an intentional design of the raised features 300. The surface roughness can be imparted by the material. The surface roughness can be imparted by manufacturing techniques.

While not to be limited by a theory, the shear stresses can cause calcium fracturing. The shear stress can be the primary mechanism for lithotripsy. The calcium can be subjected to shear stresses by the raised features 300. The calcium can be subjected to shear stresses by the lobes 120 of the balloon 100. The calcium can be subjected to stress which is parallel to the fracture plane. The sheer stress can be applied in a controlled manner by the raised features to prevent detachment of the calcium or dissections.

While not to be limited by a theory, the raised features 300 can comprise a material that is more reflective of the ultrasonic signal in the pressure wave than the balloon 100. In some embodiments, the acoustic impedance mismatch is significant and will dramatically reduce the energy transmitted to the raised features 300. There can be a small portion of the total acoustic energy that makes it through the raised features 300 compared with the balloon 100. The balloon 100 can deliver the majority of the total acoustic energy. The change in density and the speed of sound through the mediums can be summed for acoustic impedance mismatch. The material of the raised features 300 can be more dense than the calcium so the raised features 300 to deliver most of the acoustic energy traveling through the raised features 300 to the calcium. The raised features 300 can be driven by the pulsatile nature of the pressure waves. With each pressure wave, the acoustic energy that made it to the raised features 300 (small portion of the total acoustic energy) can be distributed rapidly with a lower acoustic impedance mismatch. The relative to acoustic impedance mismatch the interface geometry and the boundary conditions can have an effect. The angle of incidence can affect how impedance mismatch manifests via mode conversion, where parts of longitudinal waves may convert to shear waves in solids, modifying effective reflection. The effectiveness of transmission of the acoustic energy through the multifaceted angles of incidence along the arced interfaces at the transition of the raised feature 300 to the calcium ridge can produce a shearing wave that more effectively cracks the calcium.

While not to be limited by a theory, the raised features 300 can comprise a shape that directs the energy. The raised features 300 can channel energy into an arc. In some embodiments, the raised features 300 can channel energy into a 360° arc. The raised features 300 can restore compliance along controlled serration lines. The raised features 300 can have a shape for deep-tissue penetration. The shape of the raised features 300 can promote muti-angle distribution of energy. The raised features 300 can channel energy along a sloped side wall. The raised features 300 can channel energy into radiused arc along the radially outward surface 304.

In some embodiments, the generator can be characterized by a single parameter. In some embodiments, the generator can be characterized by two or more parameters in combination. In some embodiments, the pressure waves can generate a peak positive pressure of approximately 5 atm to 20 atm. In some embodiments, this pressure can be lower than the 50 atm or greater pressures used by other angioplasty systems. In some embodiments, the pressure wave can have a short pulse duration. In some embodiments, the pulse duration can be 1 microsecond, 2 microseconds, 3 microseconds, 4 microseconds, 5 microseconds, 10 microsecond, 20 microseconds, 30 microseconds, 40 microseconds, 50 microseconds, or any range of two of the foregoing values. In some embodiments, the pulse duration can be 0.1 milliseconds, 0.3 milliseconds, 0.5 milliseconds, 1 millisecond, 2 milliseconds, 3 milliseconds, 4 milliseconds, 5 milliseconds, or any range of two of the foregoing values. In some embodiments, the rise time can be between 1 nanosecond and 500 nanoseconds per Pascale. In some embodiments, the rise time can be between 10 nanosecond and 300 nanoseconds per Pascale. The pressure wave can have a short pulse width. In some embodiments, the pulse width can be between 0.5 microsecond and 3 milliseconds. The pressure wave can have a supersonic velocity. The pressure waves can include a high-amplitude phase and a low amplitude phase. A series of pressure wave pulses can be delivered at a frequency. There can be a frequency of the motor 830 which drives the generator, which produces a series of pulses between 1 Hz and 3 Hz. The frequency of the pulses can be 1 pulse per second (1 Hz), 2 pulses per second (2 Hz), or 3 pulses per second (3 Hz). The frequency can be changed by the user by adjusting the speed control 810. There can be frequencies associated with the pressure wave such as infrasonic frequencies less than 20 Hz. There can be frequencies associated with the pressure wave such as sonic frequencies between 20 Hz and 20 kHz. There can be frequencies associated with the pressure wave such as ultrasonic frequencies above 20 kHz. There can be frequencies associated with the pressure wave such as ultrasonic frequencies between 20 kHz and 200 kHz. There can be several acoustic bands of frequencies carried by the pressure wave. The pressure waves can be delivered continuously when the generator 800 is powered on. The pressure waves can be delivered continuously when the motor shaft 831 rotates. The system 1100 can vary the intensity of the pressure wave. The system 1100 can vary the amplitude of the pressure wave. The peak pressure can be between 1000 PSI and 2000 PSI. The peak positive pressure can be up to 20 ATM. The system 1100 can vary the periodicity of the wave pulse of the pressure wave. The system 1100 can vary parameters of the pressure wave by input by the user. The user can adjust the speed control 810. The speed control 810 can vary the periodicity of the wave pulse. The user can adjust the adjuster 822. The adjuster 822 can adjust the intensity of the pressure wave. The printed circuit board assembly 816 can adjust parameters of the pressure wave. The pressure wave can be adjusted by feedback of the magnets 812, 858. The pressure wave can be adjusted by feedback of the pressure sensor 888. The pressure wave can be adjusted by feedback of the reflective infrared sensor of the cam can 860. The pressure wave can be adjusted by a computer, processor, algorithm, and/or feedback loop. The pressure wave can be adjusted by components within the generator 800. The pressure wave can be adjusted by components outside of the generator 800 configured to transmit a signal to the generator 800. The printed circuit board assembly 816 can include software and/or hardware to receive signals to adjust the pressure wave.

The system 1100 can deliver energy in various waveform. The wave may have any suitable shape, such as, for example, square, sine, triangle, complex, compound, sawtooth, harmonic, or combinations thereof. The system 1100 can deliver a square wave. In some embodiments, the square wave can transition between two discrete levels of pressure over time at a constant frequency. In some embodiments of a square wave, the square wave can transition between more than two discrete levels of pressure over time at a constant frequency. In some embodiments a square wave, the square wave can transition between two discrete levels of pressure over time at a frequency that varies over time. In some embodiments of a square wave, the square wave can transition between more than two discrete levels of pressure over time at a frequency that varies over time.

The system 1100 can deliver a sine wave. In some embodiments, the sine wave can transition between two discrete levels of pressure over time at a constant frequency. In some embodiments of the sine wave, the sine wave can transition between more than two discrete levels of pressure over time at a constant frequency. In some embodiments of the sine wave, the sine wave can transition between two discrete levels of pressure over time at a frequency that varies over time. In some embodiments of the sine wave, the sine wave can transition between more than two discrete levels of pressure over time at a frequency that varies over time.

The system 1100 can deliver a triangular wave. In some embodiments, the triangular wave can transition between two discrete levels of pressure over time at a constant frequency. In some embodiments of the triangular wave, the triangular wave can transition between more than two discrete levels of pressure over time at a constant frequency. In some embodiments of the triangular wave, the triangular wave can transition between two discrete levels of pressure over time at a frequency that varies over time. In some embodiments of the triangular wave, the triangular wave can transition between more than two discrete levels of pressure over time at a frequency that varies over time.

The system 1100 can deliver a complex wave. In some embodiments, the complex (or compound) wave can transitions between two discrete levels of pressure over time at a constant frequency. In some embodiments of the complex wave, the complex wave can transition between more than two discrete levels of pressure over time at a constant frequency. In some embodiments of the complex wave, the complex wave can transition between two discrete levels of pressure over time at a frequency that varies over time. In some embodiments of the complex wave, the complex wave can transition between more than two discrete levels of pressure over time at a frequency that varies over time.

The system 1100 can deliver a saw tooth wave. In some embodiments, the saw tooth wave can transition between two discrete levels of pressure over time at a constant frequency. In some embodiments of the saw tooth wave, the saw tooth wave can transition between more than two discrete levels of pressure over time at a constant frequency. In some embodiments of the saw tooth wave, the saw tooth wave can transition between two discrete levels of pressure over time at a frequency that varies over time. In some embodiments of the saw tooth wave, the saw tooth wave can transition between more than two discrete levels of pressure over time at a frequency that varies over time.

The system 1100 can deliver a harmonic wave. In some embodiments, the energy contains a harmonic wave with one or more harmonic waves. In some embodiments, the harmonic wave can transition between two discrete levels of pressure over time at a constant frequency. In some embodiments of the harmonic wave, the harmonic wave can transition between more than two discrete levels of pressure over time at a constant frequency. In some embodiments of the harmonic wave, the harmonic wave can transition between two discrete levels of pressure over time at a frequency that varies over time. In some embodiments of the harmonic wave, the harmonic wave can transition between more than two discrete levels of pressure over time at a frequency that varies over time.

In some embodiments, the pressure wave can include one or more of the above waveforms.

As described herein, the generator 800 can be configured to deliver pressure waves according to certain parameters. The resulting change in the balloon 100 can correlate with the pressure of the pressure waves. The pressure wave can be any form described herein or combinations thereof.

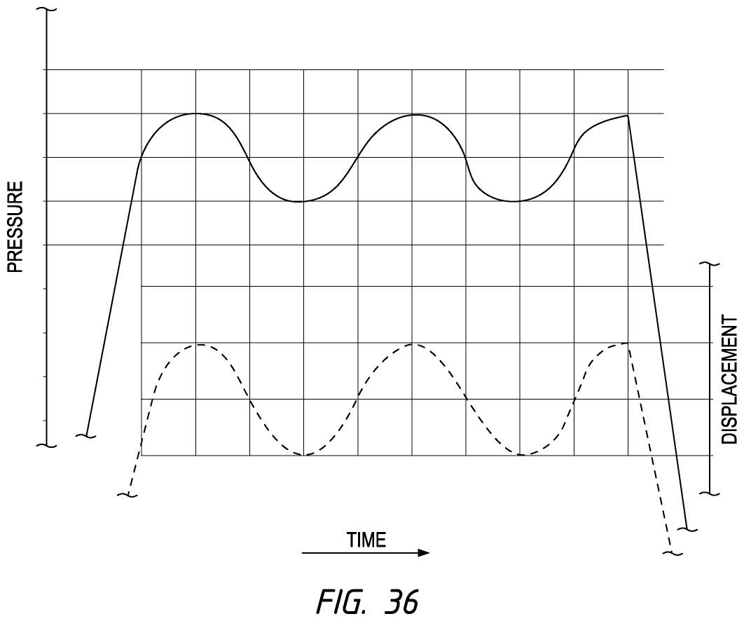

FIG. 36 illustrates the pressure wave in sinusoidal form. The pressure wave is shown in a dark solid line. The diameter displacement of the balloon 100 can correspond to the sinusoidal form of the pressure wave. The diameter displacement of the balloon 100 is shown in the dashed line.

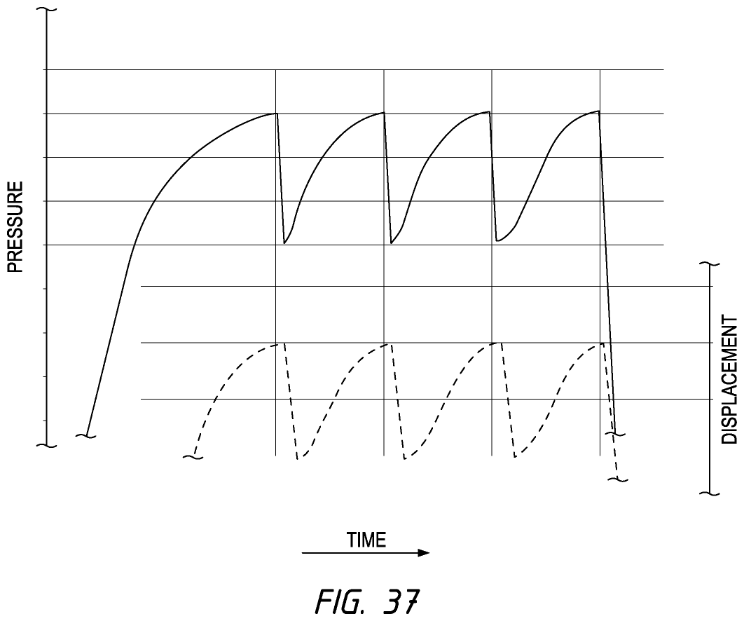

FIG. 37 illustrates that the pressure wave is in interrupted sinusoidal form. In some embodiments, upon reaching maximum pressure, pressure is immediately reduced to target minimum, then increased following sinusoidal ramp. The pattern can be repeated. The pressure wave is shown in a dark solid line. The diameter displacement of the balloon 100 can correspond to the pressure wave. The diameter displacement of the balloon 100 is shown in the dashed line.

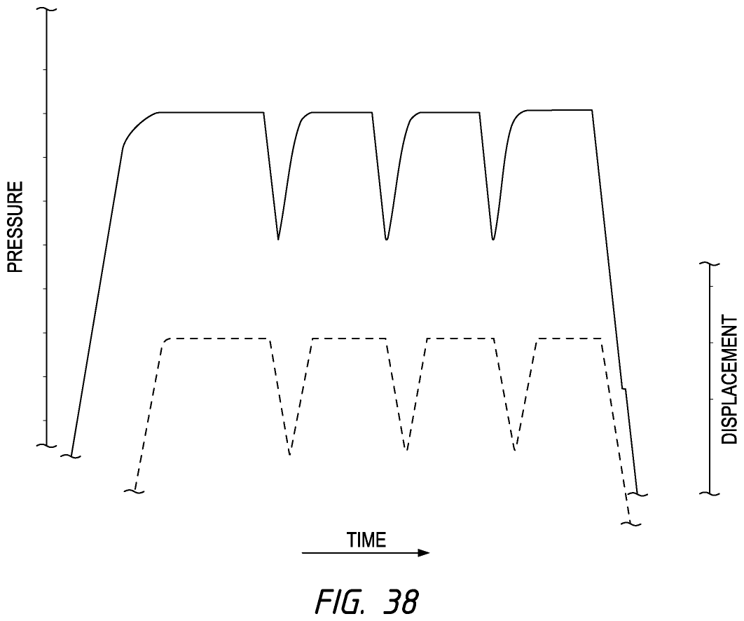

FIG. 38 illustrates that the pressure wave is increased and maximum and held (dwell) for period P. The pressure can be rapidly reduced to target minimum and immediately returned to target maximum for period P. The pattern can be repeated. The pressure wave is shown in a dark solid line. The diameter displacement of the balloon 100 can correspond to the pressure wave. The dwell of P′ can be maintained, with short deflation periods. The diameter displacement of the balloon 100 is shown in the dashed line.

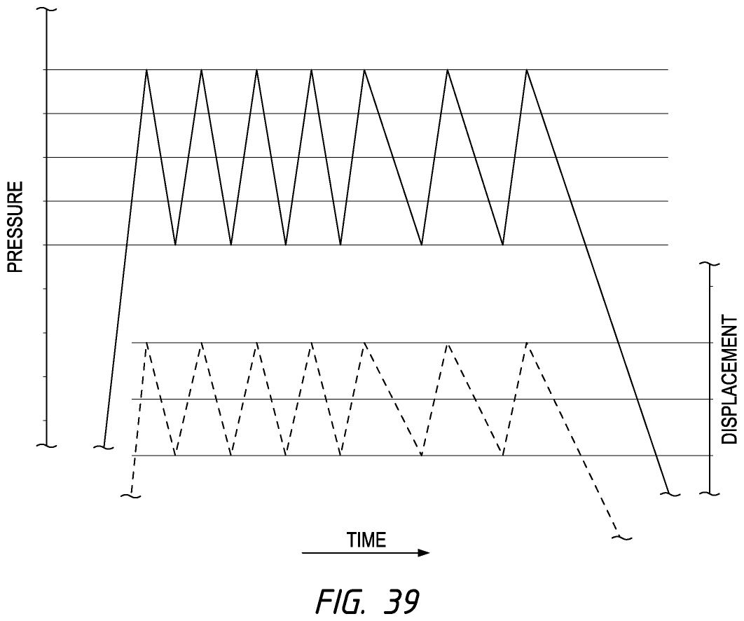

FIG. 39 illustrates that the pressure wave is increased to a maximum and rapidly reduced to a target minimum P. The pattern can be repeated. The pressure wave is shown in a dark solid line. The diameter displacement of the balloon 100 can correspond to the pressure wave. The diameter displacement of the balloon 100 is shown in the dashed line.

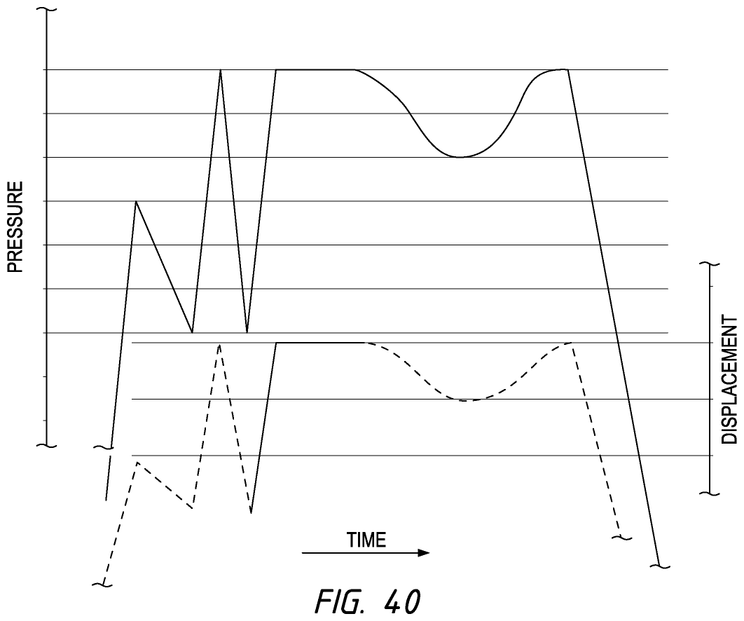

FIG. 40 illustrates that the hybrid pressure wave is applied, illustrating wide range of possible waveforms. The pattern can be repeated. The pressure wave is shown in a dark solid line. The diameter displacement of the balloon 100 can correspond to the hybrid wave. The diameter displacement of the balloon 100 is shown in the dashed line.

FIG. 41 illustrates an empirical plot of pressure and diameter according to one pattern. The waveform can be a square wave. The pattern can be repeated. The pressure wave is shown in a dark solid line. The diameter displacement of the balloon 100 can correspond to the square wave. The diameter displacement of the balloon 100 is shown in the dashed line.

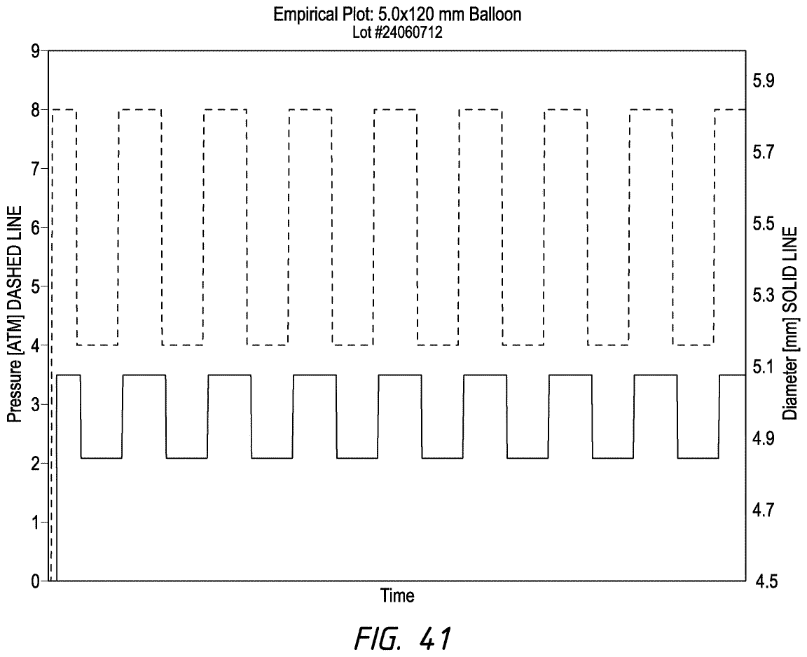

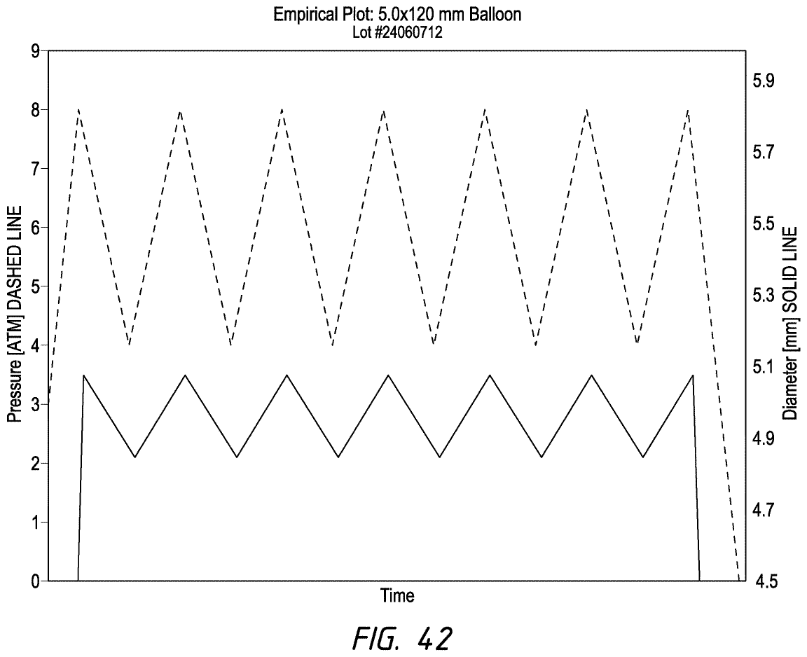

FIG. 42 illustrates an empirical plot of pressure and diameter according to one pattern. The waveform can vary between a minimum and maximum pressure. The pattern can be repeated. The pressure wave is shown in a dark solid line. The diameter displacement of the balloon 100 can correspond to the pressure wave. The diameter displacement of the balloon 100 is shown in the dashed line.

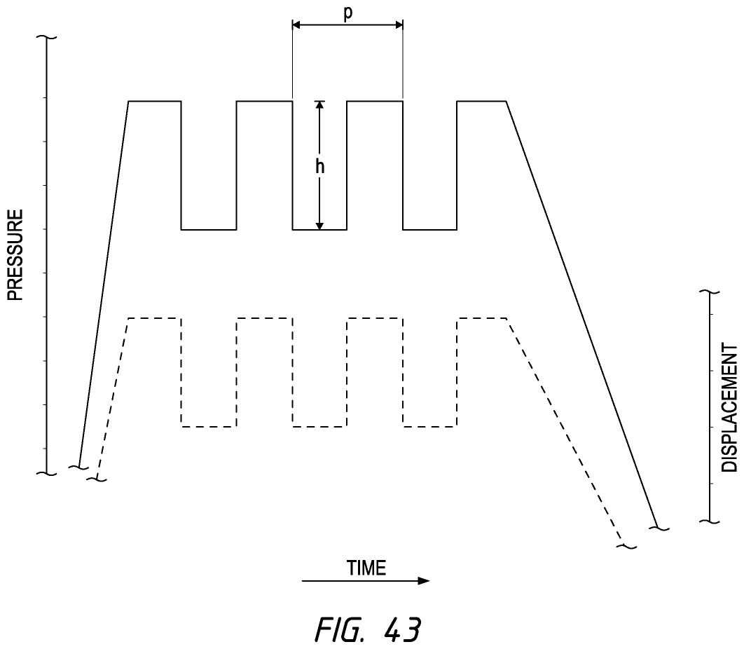

FIG. 43 illustrates a plot of pressure and diameter according to a square wave pattern of the pressure wave. The square wave can have a constant pressure high. The square wave can have a constant pressure low. The square wave can have a constant periodicity. The square wave can have a constant height. The pattern can be repeated. The pressure wave is shown in a dark solid line. The diameter displacement of the balloon 100 can correspond to the square wave. The diameter displacement of the balloon 100 is shown in the dashed line.

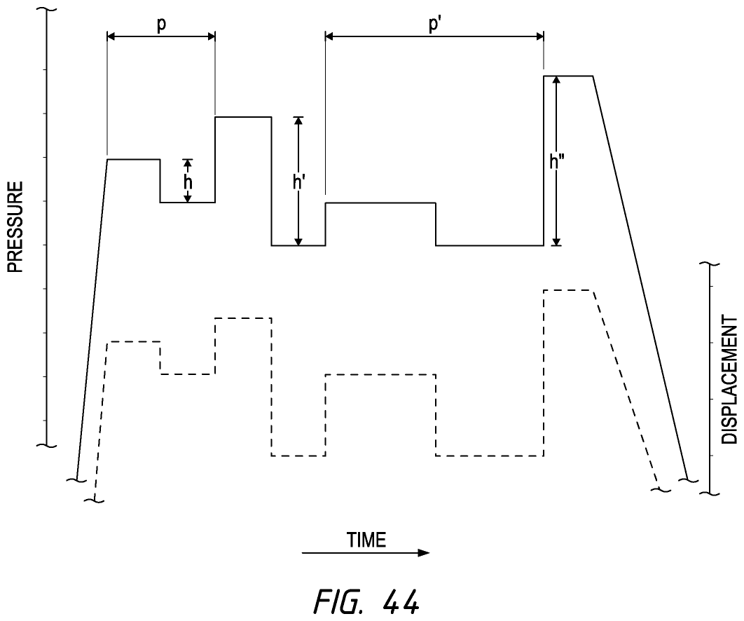

FIG. 44 illustrates a plot of pressure and diameter according to a square wave pattern of the pressure wave. The square wave can have two or more pressure highs. The square wave can have two or more pressure lows. The square wave can have two or more pressure periods. The square wave can have a variable height. The pattern can be repeated. The pressure wave is shown in a dark solid line. The diameter displacement of the balloon 100 can correspond to the square wave. The diameter displacement of the balloon 100 is shown in the dashed line.

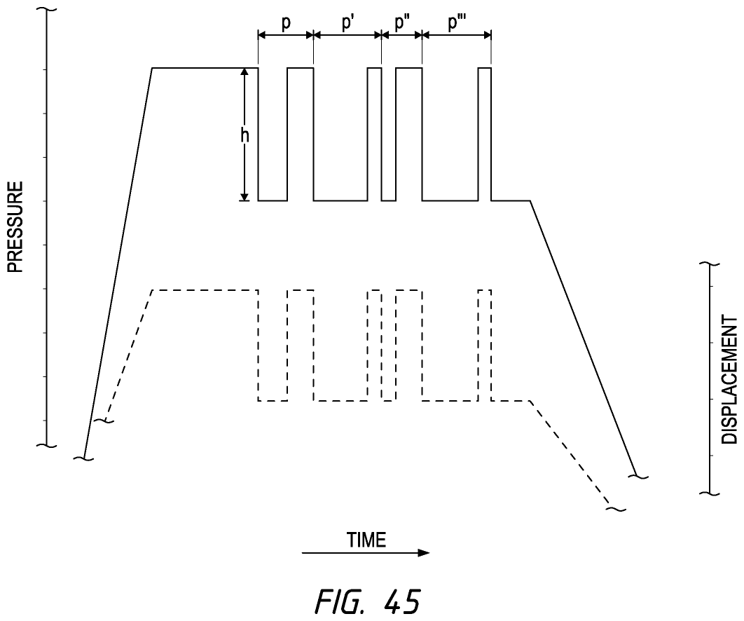

FIG. 45 illustrates a plot of pressure and diameter according to a square wave pattern of the pressure wave. The square wave can have a constant pressure high. The square wave can have a constant pressure low. The square wave can have two or more pressure periods. The square wave can have a constant height. The pattern can be repeated. The pressure wave is shown in a dark solid line. The diameter displacement of the balloon 100 can correspond to the square wave. The diameter displacement of the balloon 100 is shown in the dashed line.

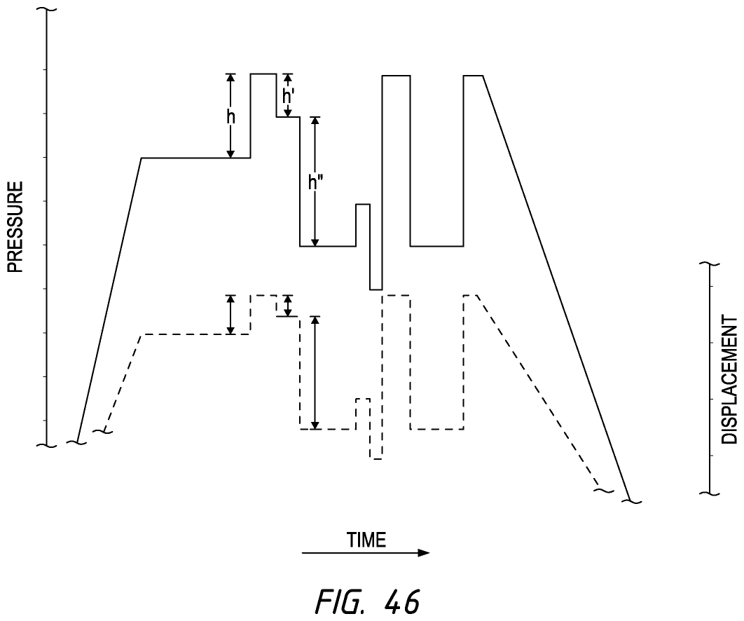

FIG. 46 illustrates a plot of pressure and diameter according to a square wave pattern of the pressure wave. The square wave can have two or more pressure highs. The square wave can have two or more pressure lows. The square wave can have two or more pressure periods. The square wave can have a variable height. The pattern can be repeated. The pressure wave is shown in a dark solid line. The diameter displacement of the balloon 100 can correspond to the square wave. The diameter displacement of the balloon 100 is shown in the dashed line.

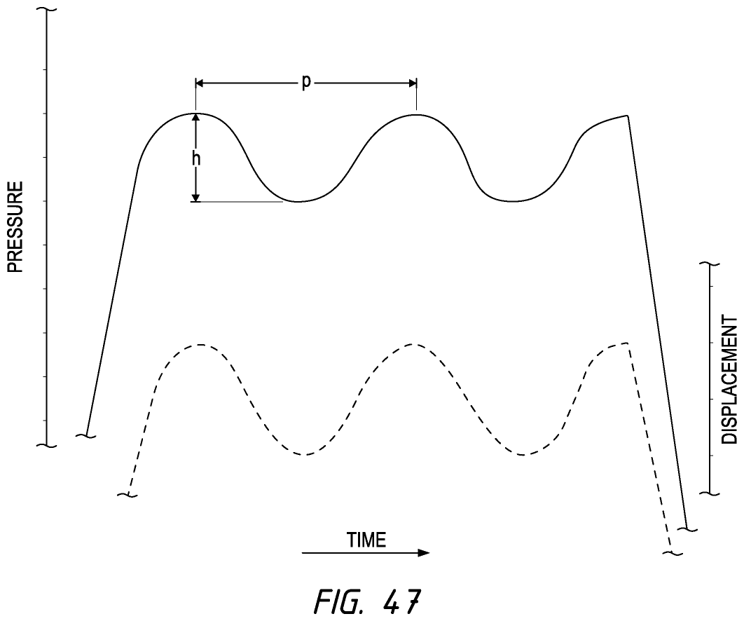

FIG. 47 illustrates a plot of pressure and diameter according to a sine wave pattern of the pressure wave. The sine wave can have a constant pressure high. The sine wave can have a constant pressure low. The sine wave can have a constant periodicity. The sinewave can have a constant height. The pattern can be repeated. The pressure wave is shown in a dark solid line. The diameter displacement of the balloon 100 can correspond to the sine wave. The diameter displacement of the balloon 100 is shown in the dashed line.

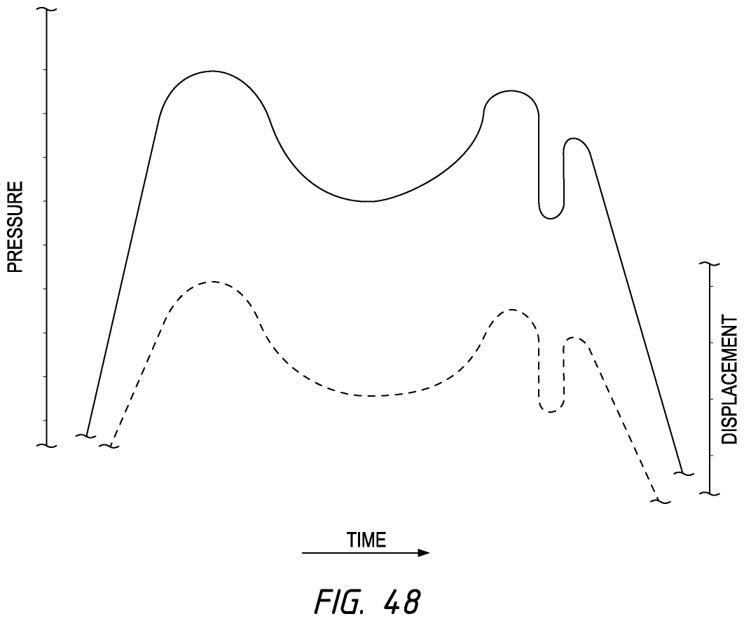

FIG. 48 illustrates a plot of pressure and diameter according to a sine wave pattern of the pressure wave. The sine wave can have two or more pressure highs. The sine wave can two or more pressure lows. The sine wave can have two or more pressure periods. The sine wave can have a variable height. The pattern can be repeated. The pressure wave is shown in a dark solid line. The diameter displacement of the balloon 100 can correspond to the sine wave. The diameter displacement of the balloon 100 is shown in the dashed line.

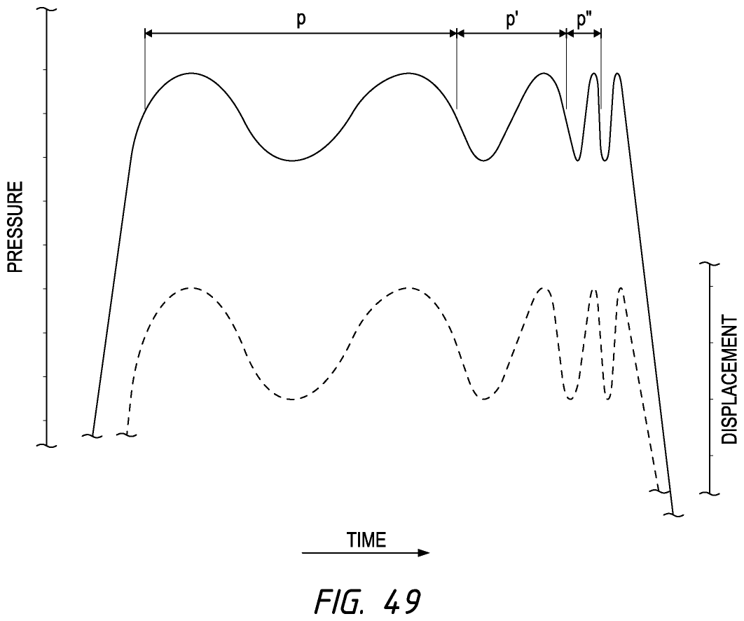

FIG. 49 illustrates a plot of pressure and diameter according to a sine wave pattern of the pressure wave. The sine wave can have a constant pressure high. The square sine can have a constant pressure low. The sine wave can have two or more pressure periods. The sine wave can have a constant height. The pattern can be repeated. The pressure wave is shown in a dark solid line. The diameter displacement of the balloon 100 can correspond to the sine wave. The diameter displacement of the balloon 100 is shown in the dashed line.

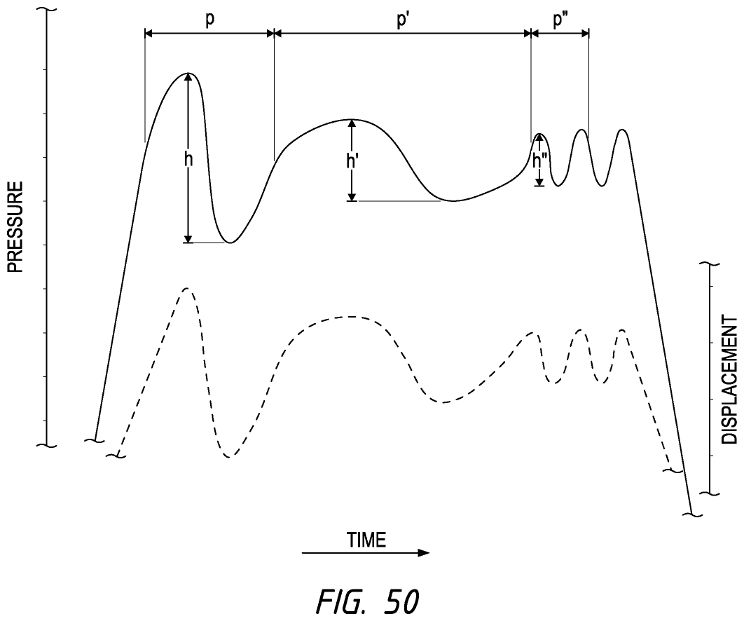

FIG. 50 illustrates a plot of pressure and diameter according to a sine wave pattern of the pressure wave. The sine wave can have two or more pressure highs. The sine wave can two or more pressure lows. The sine wave can have two or more pressure periods. The sine wave can have a variable height. The pattern can be repeated. The pressure wave is shown in a dark solid line. The diameter displacement of the balloon 100 can correspond to the sine wave. The diameter displacement of the balloon 100 is shown in the dashed line.

The balloon 100 can undergo a pressure wave cycle. In some embodiments, the system 1100 can emit a total of 80 pressure wave pulses in 8 cycles (for instance, 10 pressure wave pulses in each cycle at a frequency of 1 pulse/s). In some embodiments, the system 1100 can emit any number of pressure wave pulses per cycle including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range of two of the forgoing values. The system 1100 can pause between cycles. The system 1100 can turn off after a number of cycles. In some embodiments, the system 1100 can emit any number cycles including 1, 2, 3, 4, 5, 6, 7, 8, 9, 10, 11, 12, 13, 14, 15, 16, 17, 18, 19, 20, 30, 40, 50, 100, 150, 200, 250, 300, 350, 400, 500, 600, 700, 800, 900, 1000, or any range of two of the forgoing values. Other number of wave pulses cycles, between 1 to 1,000, are determined by the users simply turning on and off the system, and frequency, between 1 Hz to 3 Hz can be contemplated.

In some embodiments, during each cycle, the pressure of the balloon 100 can be increased by 1 atm, 2 atm, 3 atm, 4 atm, 5 atm, 6 atm, 7 atm, 8 atm, 9 atm, 10 atm, 11 atm, 12 atm, 13 atm, 14 atm, 15 atm, 16 atm, 17 atm, 18 atm, 19 atm, 20 atm, between 1 atm and 12 atm, between 5 atm and 20 atm, or any range of two of the foregoing values. In some embodiments, the base pressure can be 0 atm, greater than 0 atm, 1 atm, 2 atm, 3 atm, 4 atm, 5 atm, 6 atm, 7 atm, 8 atm, 9 atm, about 2 atm, about 1 atm, or any range of two of the foregoing values. In some embodiments, during each cycle, the peak positive pressure can be 1 atm, 2 atm, 3 atm, 4 atm, 5 atm, 6 atm, 7 atm, 8 atm, 9 atm, 10 atm, 11 atm, 12 atm, 13 atm, 14 atm, 15 atm, 16 atm, 17 atm, 18 atm, 19 atm, 20 atm, between 1 atm and 12 atm, between 5 atm and 20 atm, or any range of two of the foregoing values. The base pressure can be the same at the generator side of the membrane 884 and the balloon side of the membrane 884. The base pressure can be the static, resting pressure. The base pressure can be the same on the wet and dry side of the membrane 884. In some embodiments, at the end of each cycle, the pressure can return to the base pressure or nominal pressure. In some embodiments, during each cycle, the pressure of the balloon 100 can be increased to rated burst pressure of the balloon 100. In some embodiments, inflation can be followed by deflation of the balloon 100 by the pressure wave. In some embodiments, the pressure wave begins and ends on the lower boundary of the pressure range and spikes during the pressure wave. In some embodiments, the pressure within the balloon 100 can be dropped to zero or near zero which can allow for distal perfusion of blood.

In some embodiments, for instance designs for coronary use, the balloon 100 may be deflated after few pulses to shorten the time between perfusions, if prolonged inflation is causing ischemia. The pressure wave pulses per cycle for coronary use can be any number of pressure wave pulses per cycle including 1, 2, 3, 4, 5, 6, 7, 8, 9, 10, less than 10, less than 5, or any range of two of the forgoing values. In some embodiments, the balloon 100 can deliver a maximum of 500 pulses for coronary use, or any range described herein. In some embodiments, the balloon 100 can offer treatment of long calcified vessels or other hardened arteries.

The duration of each pressure wave can be 1 ms, 0.5 ms, 0.3 ms, 0.2 ms, 0.1 ms, 0.05 ms, 0.03 ms, 0.02 ms, 0.01 ms, 0.005 ms, 0.001 ms, or any range of two of the foregoing values. In some embodiments, the pressure wave can be in the ultrasonic range. In some embodiments, only a portion of the pressure wave rises at an ultrasonic speed range. In some embodiments, the system 1000 comprises a source of fluid 502. In some embodiments, the system 1000, 1100 can include the inflation source 400. The fluid can be compressed fluid. The fluid can be liquid. The fluid can be pressurized liquid. The fluid can be gas. The fluid can be pressurized gas. The source of fluid may be pressurized in a range from about 80 pounds per square inch (psi) to 140 psi including, for example, 120 psi.

In some embodiments, a pulse wave enhanced angioplasty balloon 100 of the system 1100 can be inflated to a pressure three to four times lower than nominal pressure, such as 2 ATM for the lower boundary of pressure, then cycled up and down by a defined upper boundary of pressure, such as 8 ATM, 9 ATM, or 10 ATM in some embodiments, producing pressure ranges of between 4 ATM to 10 ATM. The pressure range can be 1 ATM, 2 ATM, 3 ATM, 4 ATM, 5 ATM, 6 ATM, 7 ATM, 8 ATM, 9 ATM, 10 ATM, 11 ATM, 12 ATM, 13 ATM, 14 ATM, 15 ATM, 16 ATM, 17 ATM, 18 ATM, 19 ATM, 20 ATM, 21 ATM, between 1 ATM and 8 ATM, between 1 ATM and 21 ATM, or any range of two of the foregoing values.

The pressure wave can produce a shock wave. In some embodiments, the shock wave can include one or more of the following features: a non-linear single pulse with a wide frequency range (from approx. 150 kHz up to 100 MHz), high pressure amplitude (up to 150 MPa), low tensile phase (down to-25 MPa), small pulse width (of a few microseconds) and a short rise time (up to a few hundred nanoseconds). In some embodiments, the pressure wave does not produce a shock wave. In some embodiments, the pressure wave has one or more parameters associated with a shock wave. In some embodiments, the pressure wave has any parameter described herein.

In some embodiments, the pulsations of the balloon 100 with the strips 200 and raised features 300 can be 1 Hz, 2 Hz, 3 Hz, 4 Hz, 5 Hz, 10 Hz, under 5 Hz, under 3 Hz, or any range of two of the foregoing values. In some embodiments, each pulsed wave of the pulsations of the balloon 100 with the strips 200 and raised features 300 can include a portion of the sonic frequency in the ultrasonic frequency range above 20000 Hz. In some embodiments, the pulsations of the balloon 100 with the strips 200 and raised features 300 can include frequency bands in the 20 kHz, 25 kHz, 30 kHz, 35 kHz, 40 kHz, 45 kHz, 50 kHz, 55 kHz, 60 kHz, 65 kHz, 70 kHz, 75 kHz, 80 kHz, 90 kHz, 95 kHz, 100 kHz, 105 kHz, 110 kHz, 115 kHz, 120 kHz, 125 kHz, 130 kHz, 135 kHz, 140 kHz, 150 kHz, 175 kHz, 200 kHz, below 150 kHz, below 200 kHz, between 20 kHz and 150 kHz, or any range of two of the foregoing values. The balloon 100 with the strips 200 and raised features 300 can pulse any number of times while inflated including 1 time, 2 times, 3 times, 4 times, 5 times, 6 times, 7 times, 8 times, 9 times, 10 times, 100 times, 1,000 times, 2,000 times, between 1 and 100 pulses, between 1 and 50 pulses, or any range or combination of two or more of the foregoing values.

The pressure wave can accelerate inflation of the balloon 100. The balloon 100 can inflate with pressure waves in less than 0.5 seconds, 0.5 seconds, greater than 0.5 seconds, less than 1 second, 1 second, greater than 1 second, less than 1.5 seconds, 1.5 seconds, greater than 1.5 seconds, less than 2 seconds, 2 seconds, greater than 2 seconds, less than 2.5 seconds, 2.5 seconds, greater than 2.5 seconds, less than 3 seconds, 3 seconds, greater than 3 seconds, 4 seconds, 5 seconds, 6 seconds, 7 seconds, 8 seconds, 9 seconds, 10 seconds, or any range of two of the forgoing values. The balloon 100 at base pressure can orient the strips 200 and the raised features. The pressure wave can accelerate the time to reach the proper orientation of the strips 200 and the raised features 300. The pressure wave can accelerate microperforation formation. The raised features 300 can move from contact to penetration with pressure waves in less than 0.5 seconds, 0.5 seconds, greater than 0.5 seconds, less than 1 second, 1 second, greater than 1 second, less than 1.5 seconds, 1.5 seconds, greater than 1.5 seconds, less than 2 seconds, 2 seconds, greater than 2 seconds, less than 2.5 seconds, 2.5 seconds, greater than 2.5 seconds, less than 3 seconds, 3 seconds, greater than 3 seconds, 4 seconds, 5 seconds, 6 seconds, 7 seconds, 8 seconds, 9 seconds, 10 seconds, or any range of two of the forgoing values. The pressure wave can accelerate crack formation. The pressure wave can cause the crack to reach the medial plane in less than 0.5 seconds, 0.5 seconds, greater than 0.5 seconds, less than 1 second, 1 second, greater than 1 second, less than 1.5 seconds, 1.5 seconds, greater than 1.5 seconds, less than 2 seconds, 2 seconds, greater than 2 seconds, less than 2.5 seconds, 2.5 seconds, greater than 2.5 seconds, less than 3 seconds, 3 seconds, greater than 3 seconds, 4 seconds, 5 seconds, 6 seconds, 7 seconds, 8 seconds, 9 seconds, 10 seconds, or any range of two of the forgoing values. The pressure wave can accelerate calcium fracturing. The calcium can fracture with pressure waves in less than 0.5 nanoseconds, 1 nanoseconds, 1.5 nanoseconds, 2 nanoseconds, 3 nanoseconds, 4 nanoseconds, 5 nanoseconds, 10 nanoseconds, 100 nanoseconds, less than 1 microsecond, 1 microsecond, 10 microsecond, less than 1 millisecond, 1 millisecond, 10 millisecond, or any range of two of the forgoing values. The crack can from at the speed of sound. The pressure wave can crack calcium bonds in a very short time on the order of milliseconds or less. The pressure wave can crack calcium bonds in a very short time on the order of microseconds or less. The pressure wave can crack calcium bonds in a very short time on the order of nanoseconds or less. The system 1100 can crack calcium bonds with a single pressure wave. The system 1100 can crack calcium bonds with a small number of pressure waves such as less than 2 waves, 2 waves, greater than 2 waves, less than 3 waves, 3 waves, greater than 3 waves, less than 4 waves, 4 waves, greater than 4 waves, less than 5 waves, 5 waves, greater than 5 waves, less than 10 waves, less than 15 waves, less than 20 waves, or any range of two of the foregoing values.

In certain embodiments, cycle time for the pressure variability may be in a range from about 1 Hz to about 1 KHz. In some embodiments, the cycle time for the pressure wave can be 1 Hz, 2 Hz, or 3 Hz. In some embodiments, the cycle time for the pressure wave can be under 5 Hz.

In some embodiments, the amplitude of the pressure variation may be in a range from single digits to triple digits of psi, e.g., 1 psi, 2 psi, 3 psi, 4 psi, 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 200 psi, 300 psi, 400 psi, 500 psi, 600 psi, 700 psi, 800 psi, 900 psi, 1000 psi, 1100 psi, 1200 psi, 1300 psi, 1400 psi, 1500 psi, 1600 psi, 1700 psi, 1800 psi, 1900 psi, 2000 psi, 2100 psi, 2200 psi, 2300 psi, 2400 psi, 2500 psi, between 1000 psi and 2000 psi, or any range of the foregoing values. The frequency carried by the pressure wave may be in a range from about 0.1 Hz to about 10 MHz. The frequency carried by the pressure wave can be above 20 kHz, such as between 20 kHz and 150 kHz. However, in some embodiments, the higher frequencies can be dampened by the length of the catheter 600 and/or the length of the balloon 100. Therefore, some embodiments can be limited to 60 kHz or less, 100 kHz or less, 150 kHz or less, due to the length of the catheter 600 providing the pressure wave to the ballon 100.

The balloon 100 can be inflated at a target location within a vessel, and while the balloon 100 is inflated, the pulse driven mechanism can create a plurality of pulses, such as several pulses per second, at frequencies of 1 Hz, 2 Hz, 3 Hz, 4 Hz, 5 Hz, 10 Hz, or 20 Hz, between 1 Hz and 3 Hz, between 1 Hz and 5 Hz, or a summation of one or more of the foregoing frequencies. The pulse driven mechanism can create pressure waves over a period of time, such as 1 second, 2 seconds, 3 seconds, 4 seconds, 5 seconds, 6 seconds, 7 seconds, 8 seconds, 9 seconds, 10 seconds, 15 seconds, 20 seconds, 25 seconds, 30 seconds, 35 seconds, 40 seconds, 45 seconds, 50 seconds, 55 seconds, 60 seconds, 65 seconds, 70 seconds, 75 seconds, 80 seconds, 85 seconds, 90 seconds, 120 seconds, 180 seconds, or any range of two of the foregoing values. The pulse driven mechanism can create breaks between pulses, such as 0.1 seconds, 0.2 seconds, 0.3 seconds, 0.4 seconds, 0.5 seconds, 0.6 seconds, 0.7 seconds, 0.8 seconds, 0.9 seconds, 1 second, 1.5 seconds, 2 seconds, 2.5 seconds, 3 seconds, 4 seconds, 5 seconds, 6 seconds, 7 seconds, 8 seconds, 9 seconds, 10 seconds, 15 seconds, or any range of two of the foregoing values.

### Energy Transmission

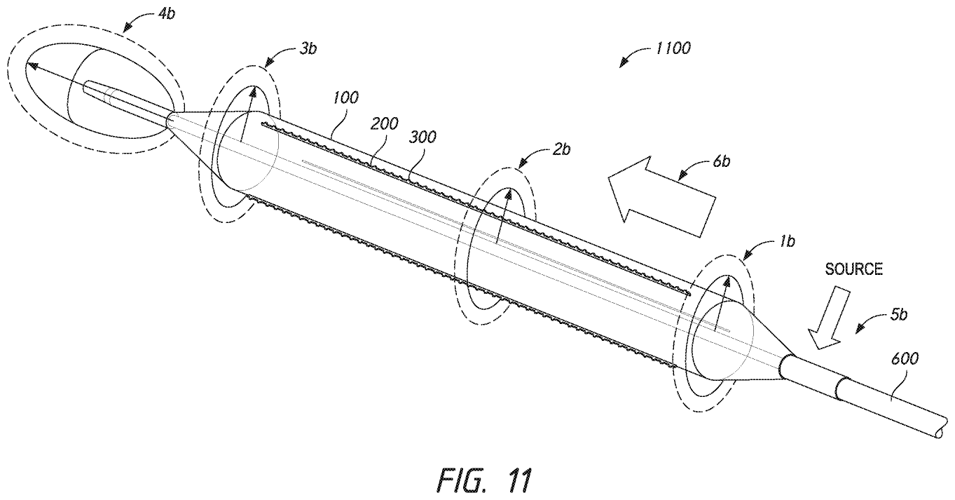

FIGS. 10-11 illustrate the balloon 100 of the system 1100. The balloon 100 can be used with the carrier 1200 as shown in FIG. 61. These figures show the emission of acoustic energy of the pressure wave from the body of the balloon 100. The illustration shows a series of four regions of emission. These four regions can be the regions where acoustic hydrophonic testing was conducted. The testing verified ultrasonic energy was emitted through the balloon 100. Minimal degradation of the acoustic signal was observed along the length of the body of the balloon 100 from region (1) to region (2) to region (3) of the body of the balloon 100. The pressure wave 100 can travel along the length of the balloon 100 without dissipation. The system 1100 can deliver consistent energy along the length of the balloon 100. The system 1100 can deliver maximum pressure that varies +/−10% along the length of the balloon 100. The system 1100 can deliver rise time that varies +/−10% along the length of the balloon 100. The system 1100 can deliver frequency that varies +/−10% along the length of the balloon 100. The system 1100 can avoid hotspots wherein energy delivery is concentrated. The system 1100 can deliver the same acoustic wave along the length of the balloon 100. The system 1100 can deliver the same acoustic wave along the circumference of the balloon 100. The pressure wave 100 can produce consistent effects along the length of the balloon. Minimal degradation of the acoustic signal was observed beyond the body of the balloon 100 at and beyond the tip at region (4). The pressure wave 100 can travel beyond the length of the balloon 100 without dissipation. The system 1100 can deliver consistent energy beyond the length of the balloon 100. The system 1100 can deliver the same acoustic wave along the length of the balloon 100 and beyond the length of the balloon 100. The pressure wave 100 can produce consistent effects beyond the length of the balloon. The pressure wave 100 can travel beyond the length of the balloon 100 to produce effects distal to the balloon 100. The pressure wave 100 can remodel the vessel beyond the length of the balloon 100. The pressure wave 100 can produce lithotripsy effects on calcium beyond the length of the balloon 100. The pressure wave can facilitate vascular remodeling. The pressure wave can cause a change in the structure of the vessel, such as a change in wall thickness, lumen diameter, and/or composition. The pressure wave can cause a change in function of the blood vessel, such as change in blood pressure, capacity, and/or flow. The pressure wave can facilitate vascular remodeling along the entire circumference of the balloon 100. The pressure wave can facilitate vascular remodeling along the entire circumference of the vessel. The pressure wave can facilitate vascular remodeling along the length of the balloon 100. The pressure wave can facilitate vascular remodeling beyond the length of the balloon as the pressure wave travels distally.

In designs where the generator 800 is proximal the body of the balloon 100, the interpretation of the design can be simplified by assuming that the pressure wave source is the point where the proximal balloon cone ends and proximal balloon leg begins. At this point source, the amount of energy entering the balloon 100 can be equal to the amount of energy that is transmitted to this point from any location proximal to this point source origin. In some embodiments, the design of the generator 800 that generates the pressure wave can be independent of the pressure wave transmitted to this assumed point source location.

In designs where a point source proximal the body of the balloon 100 can be assumed to be the pressure wave generator, the interpretation of the pressure wave event can be calculated, predicted, and measured. The four locations to be considered can be location 1a at the proximal edge of the balloon 100, location 2a at the middle of the balloon 100, location 3a at the distal edge of the balloon 100, and location 4a at the end of the distal tip of the balloon. The pressure wave was calculated, predicted, and measured at all four of these locations with minimal acoustic loss at each progressively further location from the point source. The pressure wave can experience minimal acoustic loss along the length of the balloon 100.

The designs that offer a proximal point source for the pressure wave generation can be advantageous in that the orientation of the pressure wave generation can be longitudinally oriented along the balloon length and towards the tip of the balloon 100. With this orientation, the pressure wave more uniformly fills the balloon 100. With this orientation, due to the wave speed, the pressure wave fills the body of the balloon 100 rapidly along the balloon length. As the pressure wave propagates from the proximal edge to the distal edge of the balloon 100, the pressure wave can deliver a progressive regional event horizon. The progressive regional event horizon can move through the balloon 100 and the balloon expansion can follow the wave progression. Although the time domain can be very small in which this pressure wave fills the balloon 100, the time domain can be calculated such that the filing is not uniform instantaneously. For the balloon 100 with no raised features 300 on its outer surface, for instance when the balloon 100 is used with the carrier 1200, this pressure wave progress has the ability to limit tissue injury by minimizing force loading across the entire disease zone instantaneously.

FIG. 11 illustrates the balloon 100 with strips 200 and raised features 300. This figure illustrates the emission of acoustic energy of the pressure wave from the body of the balloon 100 with strips 200 and raised features 300 longitudinally oriented on the outside of the surface of the balloon 100. The illustration shows a series of four regions. These four regions can be the regions where acoustic hydrophonic testing was conducted. The testing verified ultrasonic pressure waves were emitting through the body of the balloon 100 with strips 200 and raised features 300. Minimal degradation of the acoustic signal of the pressure wave was observed along the length from (1b) to (2b) to (3b) of the body of the ballon 100 and beyond the body of the ballon 100 at and beyond the tip (4b). The acoustic wave progressive regional event horizon can travel from the pressure wave generation source (5b) along the length of the balloon 100, illustrated by 6b.

As the pressure wave propagates from the proximal edge to the distal edge of the balloon 100 with the strips 200 and raised features 300, the pressure wave delivers a progressive regional event horizon. The progressive regional event horizon moves through the balloon 100 and the balloon expansion follows the wave progression. Although the time domain is very small in which this wave fills the balloon, the time domain can be calculated that the filing is not uniform instantaneously. The effect of this progressive regional event horizon in combination with serration angioplasty can have an magnifying effect. The magnifying effect can be driven by the wave motion through the body of the balloon 100 on each raised feature 300 of each strip 200. The magnifying effect can be in some cases in the range of 10,000×, 20,000×, 30,000×, 40,000×, 50,000× or even greater than 100,000×. Once the balloon 100 is inflated and the strips 200 with the raised features 300 have penetrated the tissue, the progressive pressure wave can produce a zipper like event that produces a linear expansion along the strips 200 and raised features 300. The progressive micro movement of each raised feature 300 in series along each strip 200 can encourage linear vessel expansion at the speed of the pressure wave event. The system 1100 can leverage the three features that can only found in intravascular lithotripsy serration technology including that a point source proximal the body of the balloon 100 transmits a pressure wave longitudinally down the body of the balloon 100, progressively expanding the balloon 100 with the strips 200 and raised features 300 to progressively penetrate into the diseased tissue with the raised features 300, and the pressure wave enters the tissue at the surface of the balloon 100, the raised features 300, and out the distal tip of the balloon 100.

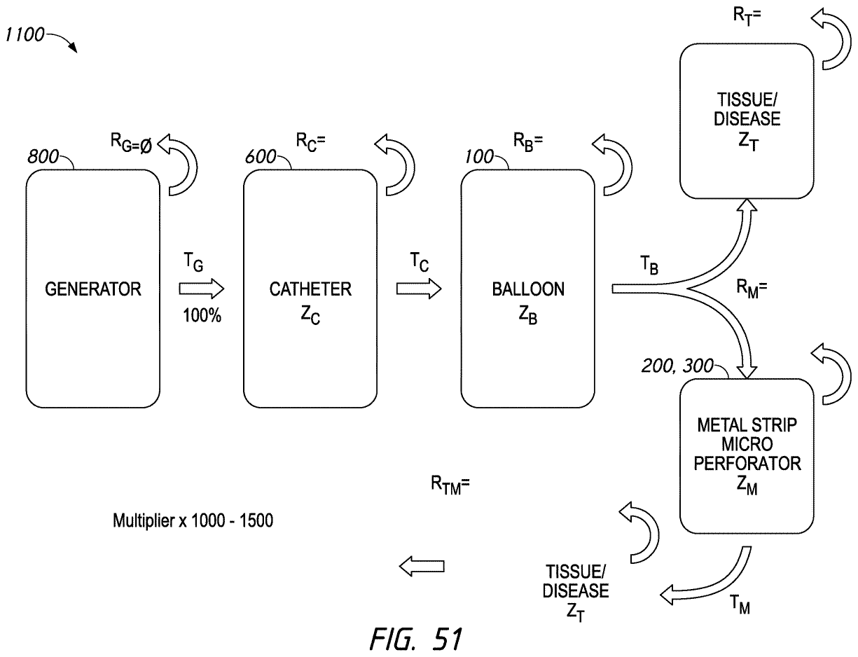

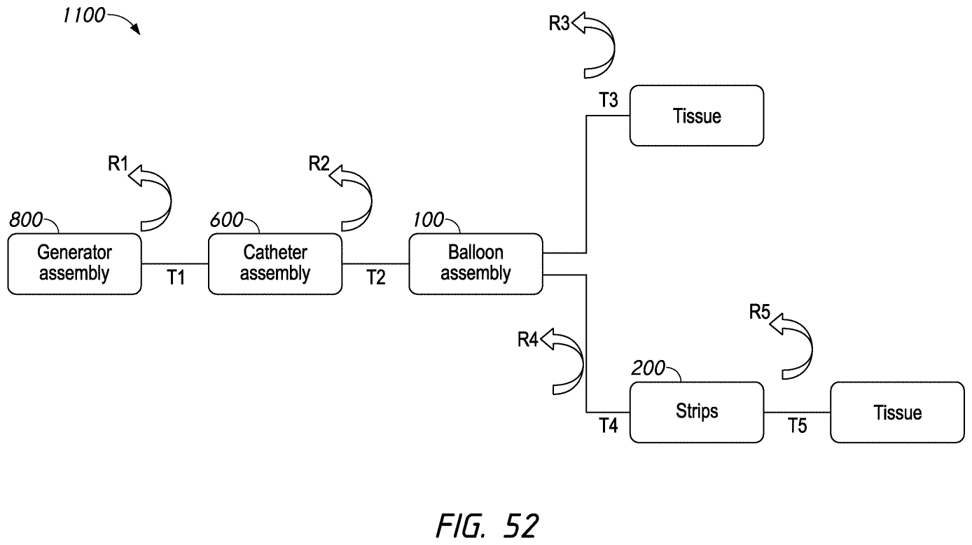

FIGS. 51-52 illustrate the system 1100. These figures show the acoustic energy transmission (T_{PowSub}) of the pressure wave from one material or design feature to the next. These figures also include the corresponding relationship of reflected energy. Some of the energy may not be transmitted but may be reflected (R_{PowSub}) instead. For acoustic waves energy there can be a power reflection component that can be calculated as:

$${R}_{PowSub} = \frac{{( {Z}_{2} - {Z}_{1} )}^{2}}{{( {Z}_{2} + {Z}_{1} )}^{2}}$$

$${T}_{PowSub} = \frac{4 {Z}_{2} {Z}_{1}}{{( {Z}_{2} + {Z}_{1} )}^{2}}$$

Where due to the energy conservation law the sum of ΣR_{PowSub} and ΣT_{PowSub} is equal to 100% of the power transmitted.

For acoustic waves energy there can be also an amplitude reflection coefficient that can be calculated as:

$${R}_{AmpSub} = \frac{{Z}_{2} - {Z}_{1}}{{Z}_{2} + {Z}_{1}}$$

$${T}_{AmpSub} = \frac{2 {Z}_{2}}{( {Z}_{2} + {Z}_{1} )}$$

Where the sum of ΣR_{Ampsub} and ΣT_{Ampsub} is equal to 100% of the intensity transmitted.

The effect of the interface between material changes can contribute to the amount of reflection and transmission of the acoustic wave. The length of the catheter 600 can contribute to the amount of reflection and transmission of the acoustic wave. As shown in FIGS. 51-52, the change from the catheter 600 to the balloon 100 can have a reflection and transmission coefficient in both power output and amplitude. These components of the waveform can be detected at various locations along the body of the balloon 100. These components of the waveform have been measured and calculated to show minimal transmission loss throughout the length of the balloon 100 from measurement (1b) to measurement (2b, 3b) and even at measurement location (4b).

When an acoustic wave encounters the interface between two different materials, part of the energy can be transmitted into the second medium while the rest can be reflected back into the first. The transmitted and reflected power can be quantified using the power reflection coefficient (R PowSub) and the power transmission coefficient (T PowSub). Similarly, the amplitude of the acoustic wave at the interface can be described by the amplitude reflection coefficient (R AmpSub) and the amplitude transmission coefficient (T AmpSub). The sum of the reflected and transmitted power coefficients can always equals 100% due to energy conservation, and likewise, the sum of the amplitude coefficients can equal the total intensity of the incident wave.

In comparing these coefficients, the power coefficients (R PowSub and T PowSub) can account for the total energy carried by the acoustic wave, which can be influenced by the acoustic impedances of the two materials. The amplitude coefficients (R AmpSub and T AmpSub) can describe the proportion of the acoustic wave's amplitude that can be reflected or transmitted at the interface. While both sets of coefficients can be affected by the impedance mismatch between the materials, the power coefficients can be more directly related to the energy transfer, whereas the amplitude coefficients pertain to the wave's displacement or pressure. Consequently, the design and material properties at the interface, such as those in the transition from the catheter 600 to the balloon 100, play a significant role in determining the balance of transmitted and reflected energy and amplitude, ultimately impacting the efficiency of acoustic energy delivery through the system 100.

Referring to FIGS. 10 and 11, the interpretation of the output of the pressure wave can be more effectively described. These figures show the progression of the pressure wave along balloon 100. The illustration shows a series of four regions. The testing showed that ultrasonic energy was emitted through the balloon 100 at each of these four regions. The pressure wave 100 can travel along the length of the balloon 100 from the proximal end to the distal end of the balloon. The pressure wave phenomena traveling along the length of the balloon 100 from proximal to distal can provide the can cause the raised features to move into and out of the vessel. This can create a zippering like effect. The vessel can have nucleation sites created by the raised features 300. These nucleation sites can provide voids that the raised features 300 can extend into and retract from. The raised features 300 can form one portion of the zipper and the hardened deposits can form the other portion of the zipper. The raised features 300 can interlock with the hardened deposits, similar to the row of raised teeth of one half of a zipper interlocks with another row of raised teeth of the other half of the zipper. The strips 200 with the raised features 300 can be pulsated with the periodicity of the pressure wave. The raised features 300 can rise and retract with each pressure wave. The raised features 300 can zipper into the hardened deposits with each pressure wave. The raised features 300 can zipper out of the hardened deposits when the balloon 100 returns to base pressure.

The raised features 300 can zipper with each pressure wave. The raised features can pulsate into and out of the hardened deposits. The frequency of the zippering can be correlated with the pulse frequency of the pressure wave. The movement of the raised features 300 can originate from the movement of the membrane 884 by the impact of the piston 878 of the generator 800. The pressure wave can travel from the reservoir 854, along the catheter 600, to the distal end of the proximal balloon leg and enter the body of the balloon 100. The pressure wave that enters the body of the balloon 100 can travel across the balloon 100. The speed of the pressure wave can be the speed of sound in the medium carrying the pressure wave. In some embodiments, the medium can be a preset mixture of saline and contrast, defined by the physician. The pressure wave can cause the expansion of the balloon 100 and thus the expansion of the raised features 300. The raised features 300 can extend into the nucleation sites at the peak positive pressure. The pressure wave can pass out of the balloon 100. The pressure within the balloon 100 can return to base pressure. The return to base pressure can cause the retraction of the balloon 100 and thus the retraction of the raised features 300. The raised features 300 can retract from the nucleation site at the base pressure. The raised features 300 can have micromovement with each pressure wave. The micromovement of the raised features 300 can cause the hammering motion when the raised features 300 contact hardened material such as plaque or other calcium deposits. The micromovement of the raised features 300 can cause cavitation to occur within the nucleation site. The zippering motion can be an in and out motion created by the rise and fall of pressure. The zippering motion can occur at the pulse frequency of the pressure waves. The hammering motion can be the impact motion when contact is made with the hardened material by the raised features 300. The zippering motion can occur without the hammering motion, in some embodiments. For instance, the raised features 300 can move into and out of the nucleation site without impacting the hardened material. The zippering motion can be less than the height of the raised features 300. The zippering motion can be on the order of microns. The zippering motion can be on the order of millimeters.

In some embodiments, all of the raised features 300 of a single strip 200 zipper into and out of the hardened material at the same time. In some embodiments, the raised features 300 of a single strip 200 move into and out of the hardened material at different times. Although the length of time to cross the balloon 100 can be very small, the pressure wave phenomena can travel in space at a measurable speed. This time for the pressure wave to pass along the length of the balloon 100 can cause the strip 200 to have a rolling motion. The strip 200 can undulate with the pressure wave as the pressure wave moves along the length of the balloon 100. The coefficients of the pressure wave can reduce in magnitude as they pass through each layer of material, however the shape and limited surface contact of the raised features 300 can provide a magnifying effect of the pressure wave. The raised features 300 can magnify the pressure wave by a factor of at least 1000. Both the transverse and longitudinal components of the pressure wave can be magnified by the narrow contact regions of the raised features 300 of the strips 200. The transverse wave component of the pressure wave can follow the edge of the raised features 300. The longitudinal wave component of the pressure wave can have a greater influence on the body of the balloon 100 and the body of the raised features 300 causing them to move in and out of the surrounding diseased tissue.

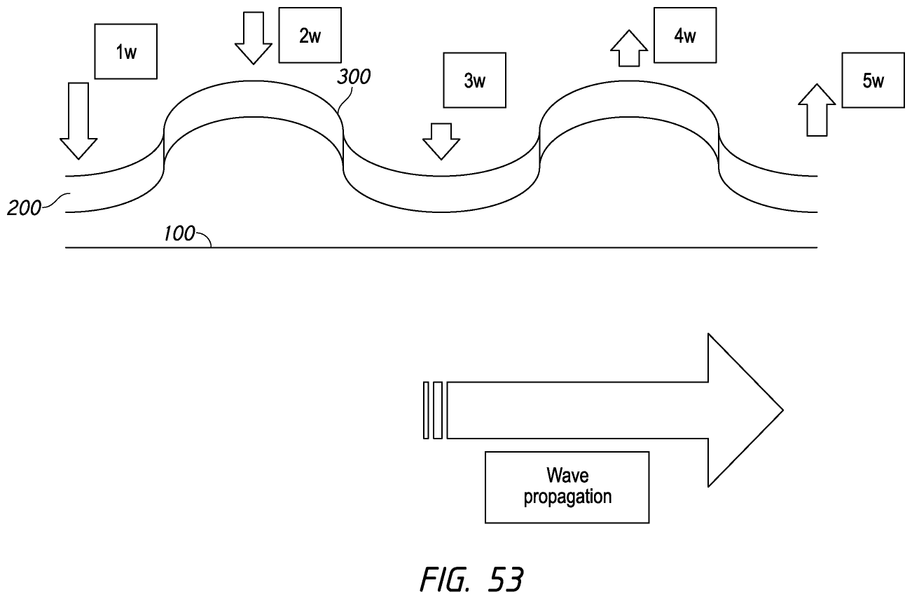

FIG. 53 illustrates the system 1100. As the pressure wave moves from the proximal to the distal regions of the balloon 100, there can be a regional effect on the surface of the balloon 100 that can be measured such as through hydrophonic testing that determines the signal along the length of the balloon and along different surfaces of the balloon. This regional effect measured was closely correlated with the pressure changes in the balloon 100. The pressure wave can carry a regional increase in pressure that moves the outer wall of the balloon 100 circumferentially, expanding the diameter in the regions where the pressure wave passes on its journey out the distal tip of the balloon 100. The dynamic nature of the pressure wave and the travel of the pressure wave in combination with strips 200 with raised features 300 offers an advantage of providing a stable platform for the raised features 300 to maintain their relative position to each other and control the directed energy pathway. In some embodiments, the motion of the strips 200 with raised features 300 may not be a one-to-one correlation to balloon expansion defined by a traveling pressure wave through an incompressible medium. As the strips 200 with raised features 300 can be rigid metal structures, the compliant nature of the balloon 100 can expand and contract at a rate greater than that of the strips 200 with raised features 300. In some embodiments, along any of the strips 200 with raised features 300, there is translation of motion correlating to the pressure wave propagation through the length of the balloon 100. As the pressure wave progresses from the proximal end to the distal end of the balloon 100, the strip 200 with raised features 300 can undergo a ripple like elevation to a new height that is established by the newly defined pressure change. Although this pressure change event can be short in nature, following the motion of the membrane 884 displacement in the reservoir 854, there can be pressure wave phenomena that travels at a known speed. The pressure wave can effect the body of the balloon 100 in a progressive nature from proximal end to distal end. As the pressure wave travels through the body of the balloon 100, the strips 200 with raised features 300 that sit proud of the body of the balloon 100 can expand at the speed of the traveling pressure wave, or in some embodiments, slightly slower due to the rigidity of the material of the strips 200 with raised features 300. The strips 200 with raised features 300 can move in an outward radial direction at least to some small degree, on the order of microns to millimeters in some embodiments. In addition to this inward and outward movement, there can be additional rolling movement of the strip caused by the propagation of the pressure wave. The undulation can be more than radially outward movement. The undulation can be successive radially outward movement along the length of the balloon 100. The raised features 300 near the proximal end can undulate outward first while the raised features near the distal end can undulate outward last. The undulation can be successive radially inward movement along the length of the balloon 100. The undulation can cause a rolling motion along the length of the strip 200. The strip 200 with the raised features 300 can have smooth wavelike motion that follows the propagation of the pressure wave. The strip 200 can rise and fall in a rolling manner with the pressure in the balloon 100. This undulation of the strip 200 with raised features 300 can be advantageous to fracture mechanics of calcium. This undulation of the strip 200 with raised features 300 can be advantageous to inducing a linear line of weakness that follows the periodic nucleation sites occurring due to the pressure wave event in the balloon 100 with the strips 200 and the raised features 300. The undulation event can effect the strips 200 with raised features 300 by rising the proximal edge of the strip 200 followed by rising the first proximal raised feature 300, then the neighboring raised features 300, then by rising additional raised features 300 until all the raised features 300 have risen to the same height. Shortly thereafter the membrane 884 of the generator 800 can return to the starting or neutral position where no volume of the reservoir 854 is displaced. In this stage, the distal edges of the strips 200 with raised features 300 begin to fall as the static pressure of the balloon 100 and the catheter 600 return to the base pressure. A reverse undulation event can then take place where the strips 200 with raised features 300 move radially inwardly starting at the distal edge of the strip 200 followed by falling the first distal raised feature 300, then the neighboring raised features 300, then followed by falling additional raised features 300 until all of the raised features 300 have fallen to the same height. This event can be repeated at a frequency equal to the speed of motor 830 which translates the cam hammer 848 and the piston 878 to push the membrane 884 outward and then releases the membrane 884 to fall back to the original position of the membrane 884 due in part to back pressure of the system 1100.

FIG. 54 illustrates time data collected to crack a calcium modeled ring for the balloon 100 with the strips 200 and the raised features 300 without the generator 800 as a wave generator. FIG. 55 illustrates time data collected to crack a calcium modeled ring for the balloon 100 with the strips 200 and the raised features 300 with the generator 800 as a wave generator. Using an external pressurized system, such as an indeflator or the inflation device 400, the system base pressure can be set to 2 atm. In FIGS. 54-55, the system pressure was increased with the generator 800 at a rate of 0.5 ml/min, which corresponded to a slow a constant ramp rate. FIG. 54 illustrates the control test. The control test was performed using the balloon 100 with the strips 200 and the raised features 300 without pulse wave generation of the generator 800. The median time to break the calcium modeled rings in the control test was 28.4 seconds. FIG. 55 illustrates the time to crack the calcium modeled rings using the balloon 100 with the strips 200 and the raised features 300 when combined with the generator 800. The median time to break with wave generation was 2.7 seconds. In some embodiments, the time to break was nearly instantaneous when the first pressure wave entered the balloon 100 with the strips 200 and the raised features, for nearly all 20 balloons tested with wave generation of the generator 800. The time to break the calcium modeled rings with the pressure wave generation was more clustered. The time to break with the pressure wave generation was more predictable. The time to break with the pressure wave generation was faster. The time to break with the pressure wave generation was faster by about 10×. While not to be limited by a theory, time to yield can be dramatically increased with the pressure wave. In some embodiments, the piston 878 can be driven forward. There can be back pressure against the membrane 884. The dry side of the piston 878 can have store energy in the form of the spring 846. The spring 846 can set the upward limit of pressure. The spring 846 can set the pressure we don't want to exceed. In some embodiments, the pressure wave can cause the calcium to yield with a single strike of the membrane 884. In some embodiments, the pressure wave can cause the calcium to yield within 1 second. In some embodiments, the pressure wave can cause the calcium to yield with two strikes of the membrane 884. In some embodiments, the pressure wave can cause the calcium to yield within 2 seconds. In some embodiments, the pressure wave can cause the calcium to yield with three strikes of the membrane 884. In some embodiments, the pressure wave can cause the calcium to yield within 3 seconds. In some embodiments, the pressure wave can cause the calcium to yield with less than five strikes of the membrane 884. In some embodiments, the pressure wave can cause the calcium to yield within 5 seconds.

FIG. 56 illustrates the median time to yield in seconds of a calcified ring. The experiment compared the use of the balloon 100 with the strips 200 and the raised features 300 as the serrated balloon with the use of the same serrated balloon with the generator 800. The time to yield was 28.4 seconds with the serrated balloon without pressure wave generation. The time to yield was 2.7 seconds with the serrated balloon with pressure wave generation. In some embodiments, the calcified ring yielded on the first pressure wave. This is a dramatic difference in time to yield. This reduction can shorten the procedure time. This reduction can shorten the time that the vessel is occluded by the balloon. While not to be limited by a theory, limiting the occlusion of the vessel can be critical for certain medical procedures to prevent tissue damage. When a vessel is blocked, tissue can be deprived of oxygen. Other reasons for limiting occlusion can include reducing ischemia, reducing arrythmias, reducing vascular damage, among other considerations. In some cases, reduced occlusion time can lead to quicker recovery for the patient including fewer complications and better outcomes. In some embodiments, reduced occlusion times can lead to shorter procedure times, reducing costs associated with the procedure. There can also be increased capacity, allowing for additional procedures per facility per day.

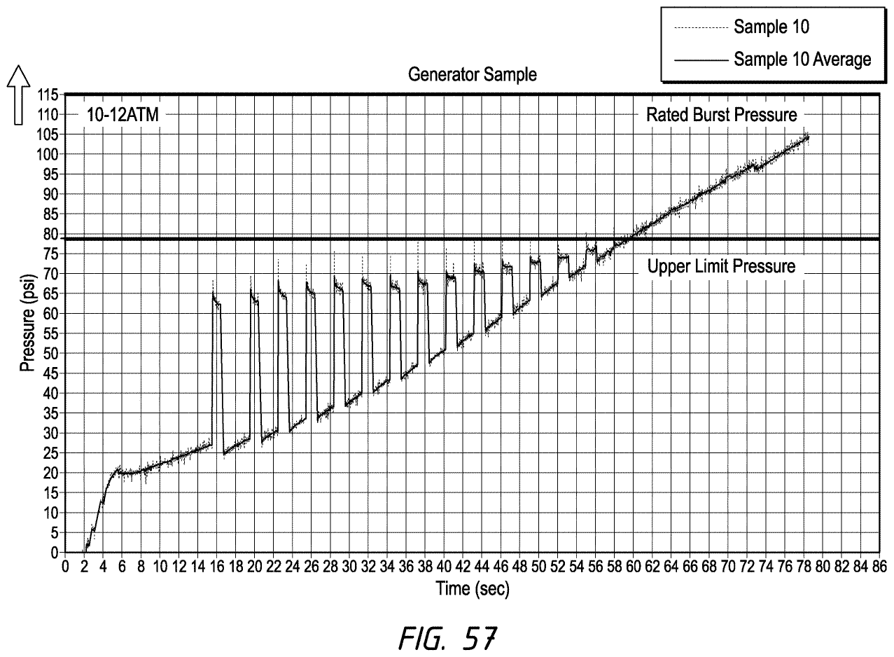

FIG. 57 illustrates data showing an upper limit of pressure entering the system 1100. The system pressure was increased with the generator 800 at a rate of 0.5 ml/min, which corresponded to a slow a constant ramp rate. Each consecutive short pulse pressure wave from the generator 800 showed a decrease in the range the from peak to peak. The balloon 100 can have an upper limit for pressure set by the generator 800. The upper limit can be about 80 psi. The orange line illustrates the sample. The blue line illustrates the sample average. The pressure spikes of the pressure wave can be below the upper pressure limit. The upper pressure limit can be set to be below the rated burst pressure of the balloon 100. The upper pressure limit can be set to be between 1 to 3 ATM below the rated burst pressure of the balloon 100.

### Calcium Fracturing Mechanics

The systems 1100 can use pressure waves to mechanically disrupt calcium deposits. The pressure waves can disrupt calcium deposits within vessel walls. The pressure waves can disrupt superficial calcium. The pressure waves can reduce calcium length between edges of the calcium. The pressure waves can reduce calcium thickness to the outer border of the calcium. The pressure waves can reduce calcium volume. The pressure waves can cause discontinuity in the calcium sheet. The pressure waves can reduce calcium density. The leading edge of the pressure wave can impart compressive stress on calcium. This compressive stress can lead to fracture. The pressure waves can create calcium plate fracture. The pressure waves can cause stress release at the junction of the calcium plate and fibrous tissue.

The pressure waves can increase concentricity of the vessel. The pressure waves can cause lumen gain. The pressure waves can increase the area of the lumen. The pressure waves can improve vessel compliance. The raised features 300 can enable controlled luminal gain. The raised features 300 can allow luminal gain at low pressure. The raised features 300 can allow luminal gain with minimal recoil.

The pressure waves can create microfractures. The pressure waves can impart compressive stress on calcium. The pressure waves can break molecular bonds of calcium. The pressure waves can disrupt calcium instead of removing calcium. The pressure waves can disrupt calcium in situ. The pressure waves can break circumferential calcium rings into smaller segments. The pressure waves can break calcium arcs into smaller segments. The pressure waves can break eccentric calcium deposits. The pressure waves can cause microfractures through the intimal layer. The pressure waves can cause microfractures through the medial layer. The pressure waves can create a fracture. The pressure waves can be reflected to further break the calcium. The pressure waves can break nodular calcium. The pressure waves can debulk nodular calcium. The pressure waves can debulk the whole coronary segment by extending beyond the balloon 100.

The pressure waves can cause fractures when encountering materials with different acoustic impedance. The pressure waves can cause fractures when encountering different impedances between soft tissue and calcification. The pressure waves can rely on a mismatch of impedance. The pressure wave can pass from the fluid of the balloon into dense calcium. The pressure wave can pass from the tissue into dense calcium. The high impedance of the calcium can cause stress within the calcium leading to fragmentation. The pressure waves can cause shearing within the calcium. The pressure waves can cause spallation within the calcium. The pressure waves can cause tensile stress within the calcium. The pressure waves can cause cavitation within the calcium. The pressure waves can enter the calcium deposit. The pressure gradient can create tensile forces which cause layer-by-layer breakage in the calcium. The pressure wave can change speed when entering calcium. The pressure wave can induce shear stress at the interface between tissue and calcium. The pressure waves can break the bonds between tissue and calcium at the interface. The pressure waves can break the brittle structure of calcium. The pressure waves can break crystalline bonds of calcium. The pressure waves can pass through the elastic bonds of tissue

The pressure wave can expand the balloon 100 radially. The radial expansion of the balloon 100 can cause strain on the vessel wall described herein. The radial expansion of the balloon 100 can facilitate vessel expansion described herein. The radial expansion of the balloon 100 can facilitate compression of the plaque described herein. The radial expansion of the balloon 100 can facilitate crack propagation described herein. The pressure wave can expand the balloon longitudinally, in some embodiments. While not to be bound by a theory, the balloon 100 can lengthen when the hardened material of the vessel limits radially expansion. The pressure wave can cause the balloon to lengthen distally. The balloon 100 can lengthen distally when radially expansion is limited. The balloon 100 can length distally beyond the distal end of the strip. In some embodiments, the balloon 100 can be constrained by the strip 100. The ballon 100 may not be able to lengthen along the length of the strip 100 which can be bonded to the balloon. The balloon 100 can lengthen proximally. The balloon 100 can lengthen proximally when radially expansion is limited. The balloon 100 can length proximally beyond the proximal end of the strip 100. The pressure waves can expand the balloon 100 both radially outward and longitudinally in some embodiments. The pressure wave can expand the strip 200 with raised features 300 radially. The radial expansion of the strip 200 with raised features 300 can cause the zippering effect described herein. The radial expansion of the strip 200 with raised features 300 can cause the hammering effect described herein. In some embodiments, the pressure wave does not expand spherically outwards from an emitter. The pressure waves can be generated proximal to the balloon 100 and travel distally. The pressure waves can generate an acoustic shockwave. The pressure waves can be unfocused. The pressure waves can produce lower energy flux density compared with other systems. The pressure waves can be generated along the length of the balloon 100, in proximity to the vascular calcium. The pressure waves can disrupt and fracture the calcium within the vessel wall. The pressure waves can change vessel compliance. The pressure waves can maintain the structure of the vessel including fibrous tissues of the vessel.

The system 1100 can deliver pressure waves circumferentially. The system 1100 can modify vascular calcium. The system 1100 can deliver pressure within a range suitable for tissue delivery. The system 1100 can deliver pressure to apply compressive force to modify calcium. The system 1100 can deliver pressure within a tensile stress range. The system 1100 can deliver pressure waves without thermal generation of emitters. The system 1100 can deliver pressure without thermal injury. The system 1100 can deliver pressure waves without multiple emitters. The system 1100 can deliver pressure waves from a source outside of the body of the balloon 100. The source can be conceptualized as a theoretical source distal to the distal end of the balloon. The system 1100 can deliver pressure waves from the theoretical source outside of the body of the balloon 100. The system 1100 can deliver pressure waves that originate at the generator 800. The system 1100 can deliver pressure waves that originate at the membrane 884. The system 1100 can deliver pressure waves for treatment along the length of the balloon 100. The system 1100 can deliver pressure waves for treatment beyond the length of the balloon 100. The system 1100 can provide support to the vessel via the balloon 100. The balloon 100 can apply a pressure to the vessel to minimize deformation of the vessel during delivery of the pressure waves. The balloon 100 can allow the acoustic waves to travel therethrough. The balloon 100 can provide a large surface area for transmission of the pressure waves into the tissue. The balloon 100 can provide a transmission surface to the vessel wall.

### Methods

The balloon 100 can be used in any method described herein. FIG. 58A-58E is a series of illustrations showing inflation of the balloon 100 with strips 200 and raised features 300. In some embodiments, the balloon 100 can be inflated by inflation device 400 connected to the reservoir 854. The balloon 100 can be inflated to the base pressure. The pressure wave can be generated. In some methods, the pressure wave can be generated during any step of methods described herein. In some methods, the pressure wave can be generated when fluid fills the balloon 100. In some methods, the pressure wave can be generated during filling of the balloon 100. In some methods, the pressure wave can be generated after filling of the balloon 100. In some methods, the pressure wave can be generated before the balloon 100 reaches base pressure. In some methods, the pressure wave can be generated after the balloon 100 reaches base pressure. In some methods, the pressure wave can be generated before the balloon 100 contacts the vessel 10. In some methods, the pressure wave can be generated while balloon 100 contacts the vessel 10. In some methods, the pressure wave can be generated after the balloon 100 contacts a vessel 10. In some methods, the pressure wave can be generated after the balloon 100 reaches a base pressure. In some methods, the pressure wave can be generated before the raised features 300 penetrate the vessel. In some methods, the pressure wave can be generated while the raised features 300 penetrate the vessel. In some methods, the pressure wave can be generated after the raised features 300 penetrate the vessel. The pressure wave can be generated during any period of the methods described herein.

The system 1100 can deliver pressure waves to the balloon 100 during one or more steps described herein. The system 1100 can deliver pressure waves to the balloon 100 during one or more steps shown in FIGS. 58B-58E in some methods. The system 1100 can deliver pulsatile pressure to the balloon 100 before one or more steps shown in FIGS. 58A-58E in some methods. The system 1100 can deliver pulsatile pressure to the balloon 100 after one or more steps shown in FIGS. 58A-58E in some methods. The system 1100 can be designed for breaking up calcified lesions in vessels. In some embodiments, the system 1100 generates a pressure wave within the fluid-filled balloon 100. In some embodiments, the system 1100 generates a subsonic pressure wave. In some embodiments, the system 1100 generates a ultrasonic pressure wave. In some embodiments, the system 1100 comprises an intravascular lithotripsy system. The pressure wave can travel through the fluid in the catheter 600 and through fluid material as a fluid wave. The pressure wave can travel through the balloon 100 and through the strip 200 with the raised features 300 to interact with the calcification. The pressure wave can travel through the fluid medium of the reservoir 854, the catheter 600, and the balloon 100. The pressure wave can travel through the solid medium of the balloon 100 to interact with the vessel 10 and/or calcification. The pressure wave can travel through the solid medium of the strips 200 with raised features 300 to interact with the vessel 10 and/or calcification.

In some methods, there can be a set periods defining specific events. The periods can be independent of the waveform or other parameters of the pressure wave delivered. The pressure wave can be delivered during any of the periods described herein. . . . In some embodiments, some of the periods can be defined in the time domain. In some embodiments, some of the periods can be defined in the pressure domain. For instance, one period can be described as the unfolding period as described further herein. This unfolding period can be a time domain period that takes a defined amount of time to occur. The event that occurs during the unfolding period includes the series of steps to enable the strips 200 that lie within the folds of the balloon 100 during delivery to rotate upward and face perpendicular to the wall of the vessel in preparation for penetration into the vessel intima as described herein. In some embodiments, the time for the balloon 100 to move from its crimped or pleated delivery state to its pressurized fully expanded state can be typically between 1 seconds to 60 seconds without the use of pressure waves. In some embodiments, the time period from the unfolding period to the full expansion period can occur in less time with the use of pressure waves, typically as short as 0.2 seconds to 1 seconds. In some embodiments, the time period from the unfolding period to the for the full expansion period can be 0.2 seconds, 0.5 seconds, 1 second, 2 seconds, 3 seconds, 4 seconds, 5 seconds, 10 seconds, 15 seconds, 20 seconds, 25 seconds, 30 seconds, 35 seconds, 40 seconds, 45 seconds, 50 seconds, 55 seconds, 60 seconds, or any range of two of the foregoing values. The rise in pressure from the unfolding period to the full expansion period can be typically between 0.1 atmosphere and 2 atmosphere without the use of pressure waves. In some embodiments, the rise in pressure from the unfolding period to the during full expansion can be between 1 atmosphere and 4 atmospheres without the use of pressure waves. In some embodiments, the rise in pressure from the unfolding period to the during full expansion can be between 1 atmosphere and 12 atmospheres with the use of pressure waves. In some embodiments, the unfolding zone occurs prior to the delivery of the pressure wave. The pressure wave can be delivered once fluid fills the balloon 100 in some methods. The pressure wave can include an ultrasonic frequency. This controlled increase in pressure by the pressure wave can be generated by the generator 800 described herein.

Positioning within the Vessel

The balloon 100 can be used at any position relative to the occlusion. The balloon 100 can be deflated when moved within the vessel. The balloon 100 can be positioned at the treatment site. The balloon 100 can be brought to a location within a vessel with a stenosis. The balloon 100 can be used with any method described herein.

The balloon 100 can be positioned proximally to the treatment site. The balloon 100 can be positioned proximally to the occlusion. The balloon 100 can be positioned upstream from the target location. In some methods, the balloon 100 may not be able to access the target location. In some methods, the balloon 100 may have a diameter larger than the diameter of the occlusion. The vessel can include partial occlusion. The vessel can include mural calcification. The vessel can include deposits of calcium within the vessel. The vessel can include deposits of calcium within the media. The vessel can include calcium that form curvilinear deposits. The vessel can include ring-like calcium. The vessel can include calcium within the intima. The vessel can include deposits such as plaque or other calcifications on the wall of the vessel. The deposits can partially occlude the vessel.

### Delivery Period

In some embodiments, a pre-angioplasty catheter can be used for delivery of the balloon 100 across tight lesions. The pre-angioplasty catheter can be used for balloon dilatation of the vessel prior to delivery of the balloon 100. The balloon 100 can be used with any intravascular device or method to reach the treatment site.

FIG. 58A illustrates the pleated and wrapped balloon 100. The balloon can be folded to a low profile. The balloon 100 can include one or more pleats, for example one pleat, two pleats, three pleats, four pleats, five pleats, six pleats, or any range of two of the foregoing values. The number of pleats can correspond to the number of strips 200. In the illustrated embodiment, the balloon 100 can include three strips 200 and the balloon 100 includes three pleats. The pleats can be configured to at least partially cover the strip 200. The pleat can cover the height of the strip 200. The pleat can cover the tip of the strip 200. The pleat can overhang the strip 200. The strip 200 can be disposed between the pleat and a surface of the balloon. The strip 200 can be disposed within a pocket. The strips 200 can be contained within the folds of the balloon 100. The pleat can form a wing 110. The wing 110 can a portion of the balloon 100 that overlies a portion of the strip 200. The wing 110 can be a flap of material of the balloon 100. The wing 110 can be configured to be inflated as described herein.

The strips 200 can be at least partially covered by the balloon 100 during insertion. The balloon 100, in particular the wing 110, can serve as a buffer between the vessel lumen and the strips 200 during delivery or retrieval. The balloon 100 can cover the tip of the strip 200. The tip of the strip 200 can be covered to protect the raised features 300. The raised features 300 can be covered to protect the vessel wall during insertion and retrieval.

The strip 200 can be delivered in a tangential orientation. The tangential orientation can allow the strips 200 to lie against a surface of the balloon 100. The tangential orientation can allow the strips 200 to have a low profile delivery configuration. The tangential orientation can allow the raised features 300 to be covered by the wing 110. The tangential orientation can reduce the diameter of the catheter for delivery and retrieval. The tangential orientation can contain the strips 200 within the folds or pleats of the balloon 200. The tangential orientation can allow the strips 200 to lie flat. The strips 200 can be near or against an inner member 150 of the balloon 100. The inner member 150 can include a lumen for guidewire insertion. The low profile diameter of the balloon 100 with the strips 200 can include the thickness of the strips 200. In some embodiments, the low profile diameter of the balloon 100 with the strips 200 does not include the height of the strips 200. The low profile diameter of the balloon 100 with the strips 200 can be smaller due to the tangential orientation of the strips 200 for delivery. In some embodiments, the delivery diameter of the balloon 100 with the strips 200 would be greater if the strips 200 with the raised features 300 were oriented perpendicularly. The delivery diameter balloon 100 with the strips 200 can be smaller than the vessel diameter. The balloon 100 with the strips 200 and the raised features 300 can be delivered to the treatment site within the vessel in the low profile configuration. The balloon 100 with the strips 200 and the raised features 300 can be delivered to a treatment site with the strips 200 in the tangential orientation.

The location of the strips 200 within the fold of the balloon 100 can offer an advantage during the inflation cycle as described herein. The location of the strips 200 within the fold of the balloon 100 can enable the strips 200 to rotate from their tangential orientation illustrated in FIG. 58A. The strip 200 can rotate once the balloon 100 is located at the treatment site. The strip 200 can rotate once the balloon 100 further inflates. The strips 200 have a tangential orientation for delivery. The tangential orientation for delivery can allow the strips 200 to be tucked within the fold the balloon 100.

The wings 110 of the balloon 100 can offer an advantage during the inflation cycle as described herein. The balloon material 100 can overlie the strip 200. The balloon material can overhang the tip of each raised feature 300. The balloon material that forms the pleats or wings 110 is shown. In some embodiments, the wing 110 can extend beyond the raised feature. In some embodiments, the wing 110 does not to extend past the neighboring strip 200. In some embodiments, the wing 110 only covers one or a portion of one strip 200. In some embodiments, the wing 110 does not fully cover a strip 200. In some embodiments, the wing 110 fully covers a strip 200. In some embodiments, the wing 110 does not fully cover another strip 200.

### Initial Expansion Period

FIG. 58B illustrates the initial expansion period (or unfolding period) with fluid entering the balloon 100 and expanding the balloon 100. The inflation device 400 can supply fluid to the reservoir 854 which can fill the balloon 100. The fluid can be any medium described herein. The fluid can be contrast material. The inflation device 400 can supply fluid at low pressures during the initial expansion event, such as a pressure of 0.5 ATM, 1 ATM, 1.5 ATM, 2 ATM, 2.5 ATM, 3 ATM, under 0.5 ATM, under 1 ATM, under 1.5 ATM, under 2 ATM, under 2.5 ATM, under 3 ATM, at least 0.5 ATM, at least 1 ATM, at least 1.5 ATM, at least 2 ATM, at least 2.5 ATM, at least 3 ATM, or any range of two of the foregoing values. During this phase of the expansion, the strips 200 with the raised features 300 can begin the rotation to the perpendicular orientation. The treatment site within the vessel 10 can be illustrated. For instance, the vessel 10 can include an occlusion with a 2 mm inner diameter The vessel 10 can represent a surface that might limit expansion and limit rotation of the strip elements 200. As the balloon 100 fills with fluid, the pressure within the balloon 100 can be negligible but enough to begin to unfold the balloon 100. As the balloon 100 unfolds, the wings 110 of balloon 100 that buried the strip 200 can peel back. At the same time, the regions of the balloon 100 between the strips 200, where minimal resistance to filling can be present, begin to fill and begin to apply gentle pressure to the inside wall of the strips 200 facing the inner member 150 of the balloon 100.

FIG. 58B illustrates the initial expansion of the balloon 100. The balloon 100 can be positioned within the vessel 10. The wall of the vessel 300 is illustrated as a circle, but the vessel wall can have any cross-sectional shape. The fluid can begin to enter the balloon 100. The fluid can expand the balloon 100. The balloon 100 can form lobes 120. The lobes 120 can be located between the strips 200. The balloon 100 can have any number of lobes, for example one lobe, two lobes, three lobes, four lobes, five lobes, six lobes, or any range of two of the foregoing values. The lobes 120 can expand. When the lobes are configured as shown in FIGS. 58B and 58C, the lobes 120 can pulsate at the frequency of the pressure wave. The pulsating lobes 120 can further promote the strips 200 to penetrate the vessel. The pulsating lobes 120 can deliver ultrasonic energy into the vessel. The pressure wave together with the pulsating lobes 120 can raise the strips 200 into hardened calcium. The raised strips 200 can contact the calcium sheet and generate a node or region of weakness. The region of weakness in the calcium sheet promotes the translation of ultrasonic energy through out the calcium sheet generating a regional sheering force in the calcium. The sheering force weakens the calcium sheet and promotes the pulsating lobes 120 to deliver sheering forces to the calcium sheet in regions where no strips 200 are present. A greater amount of the ultrasound can be transmitted to the strips from the side walls of the lobes 120 facing the strips 200 in FIGS. 58B and 58C. The increase in regional surface contact or adjacent lobe area that can transmit the ultrasonic signal to the strip can be as high as 5× the amount of the base area contact of the strip 200. This increase in ultrasonic energy transfer can occur during the initial phase of strip-tissue penetration. The lobes 120 can expand when supplied with fluid from the inflation device 400. The lobes 120 can expand from a position not in contact with the vessel 10 to a position contacting the vessel 10. The balloon 100 can inflate until the lobes 120 rest against the vessel 10. Continued filling of the balloon 100 can cause a greater surface area of the lobes 120 to contact the vessel 10. The fluid enters the balloon 100 and expands the lobes 120. The lobes 120 can uniformly expand. The lobes 120 can simultaneously expand. The lobes 120 can non-uniformly expand. The lobes 120 can sequentially expand. The lobes 120 can passively expand based on the geometry of the vessel 10. The lobes 120 can expand based on areas of the balloon 100 with low resistance. During inflation, the lobes 120 of the balloon 100 can contact the vessel 10. The lobes 120 of the balloon 100 can contact a percentage of the circumference of the vessel 10. The lobes 120 of the balloon 100 can contact a majority of the circumference of the vessel 10.

The strips 200 can rotate. The strip 200 can rotate as the balloon 100 expands. The strips 200 can also rotate as the balloon 100 deflates, as further described herein. The direction the strips 200 rotate can be defined by the orientation of how the balloon 100 and strips 200 are pleated. The direction the strips 200 rotate can be determined by whether the balloon 100 is being inflated or deflated. For the following description, in relation to FIGS. 58A-58E, the strips 200 can be oriented tangentially pointing counterclockwise. In the series from FIG. 58A to FIG. 58E, the balloon can be undergoing inflation and the strips 200 with the raised features 300 can rotate from the transverse orientation to the perpendicular orientation by rotating clockwise during inflation. The strips 200 with the raised features 300 can rotate from the perpendicular orientation to the transverse orientation by rotating counterclockwise during deflation. In other embodiments, the strips 200 can be oriented tangentially pointing clockwise. In that embodiment, the strips 200 rotate from the transverse orientation to the perpendicular orientation by rotating counterclockwise during inflation. The strips 200 with the raised features 300 can rotate from the perpendicular orientation to the transverse orientation by rotating clockwise during deflation. The strips 200 can be able to rotate in either direction due to the orientation of the pleats placed during the manufacturing process. The pleats can help to define the direction the strips 200 point either clockwise or counterclockwise. The pleats can help reliably unfold and rotate the strips 200 during inflation. The pleats can help reliably refold and rotate the strips 200 during deflation. The strips 200 can rotate clockwise or counter-clockwise. The strips 200 can rotate out and back during the inflation and deflation cycles. The strips 200 can rotate in a first direction during inflation. The strips 200 with the raised features 300 can rotate in a second, opposite direction during deflation. The strips 200 with the raised features 300 can reliably and repeatedly rotate from the transverse orientation to the perpendicular orientation, and vice versa. The inflation and deflation cycles can occur more than once during a procedure. The repositioning of the strips 200 under the pleat or the balloon wing 110 can occur many times in a single procedure.

The strips 200 can rotate from the tangential orientation to a less tangential orientation. The strips 200 with the raised features 300 can rotate from the tangential orientation to a more perpendicular orientation. The strips 200 with the raised features 300 can rotate relative to the inner member 150 of the balloon 100. The strips 200 with the raised features 300 can rotate under the influence of the expansion of the balloon 100. The strip 200 with the raised features 300 can begin rotation to the perpendicular orientation. The strip 200 can rotate to the wall of the vessel 10. The raised feature 300 of the strip 200 can have the radially outwardly facing surface 304. The radially outwardly facing surface 304 of the raised feature 300 can be unhoned. The strip 200 can rotate until the radially outwardly facing surface 304 contacts the wall of the vessel 10. The vessel 10 can have any diameter. The lobes 120 can be against the vessel 10. The radially outwardly facing surfaces 304 of the raised features 300 can be against the vessel 10. The vessel 10 can limit expansion of the lobes 120. The vessel 10 can limit rotation of the strips 200.

The balloon 100 can continue to fill with fluid. The balloon 100 can expand when supplied with fluid from the inflation device 400. The fluid can flow from the reservoir 854 to the catheter 600 and into the balloon 100. The fluid can be any fluid, such as liquid or gas. The fluid can be contrast material. The pressure in the balloon 100 can be low, e.g., negligible, less than 0.5 atm, less than 1 atm, less than 2 atm. The low pressure can allow the balloon 100 to unfold. The balloon 100 can form bigger lobes 120 as the balloon 100 fills. The wings 110 of the balloon 100 can begin to pull back as the balloon 100 fills. The wings 110 of the balloon 100 can fill with fluid. The wings 110 of the balloon 100 can begin to merge with the lobes 120 of the balloon 100. The wings 110 of the balloon 100 can uncover the radially outwardly facing surfaces 304 of the raised features 300 of the strips 200. The wings 110 of the balloon 100 can uncover the side or height of the strip 200. The wings 110 of the balloon 100 can uncover the strip 200.

The lobes 120 of the balloon 100 can continue to expand. The lobes 120 can expand when supplied with fluid from the inflation device 400. The lobe 120 can be the region of the balloon 100 between adjacent strips 200. The lobes 120 can experience minimal resistance to filling. In some embodiments, the filling of the lobes 120 is not constrained by the strips 200. The lobe 120 can have less resistance to filling than the wing 110. As the wing 110 merges with the lobe 120, the wing 110 can merge with the lobe 120. The wing 110 can migrate to the region between adjacent strips 200. The wing 100 can migrate to uncover the strip 200. The fluid can fill the areas with minimal resistance to filling. The lobes 120 can apply a pressure to the wall of the vessel 10. The lobe 120 can apply a pressure to the strip 200. The lobe 120 can apply a pressure to the inside wall of the strip 200. The lobe 120 can apply pressure to the inside wall of the strip 100 that faces the inner member 150 of the balloon 100. The inside wall of the strip 100 can be a sloped sidewall of the strip 200. The inside wall of the strip 100 can be a longitudinally oriented wall. The lobe 120 can apply pressure to rotate the strip 200. The lobe 120 can apply pressure to orient the strip 200 with the raised features 300 perpendicular to the inner member 150 of the balloon 100.

While not to be limited by a theory, there may be a portion 130 of the lobe 120 that causes rotation. The portion 130 can be located between the surface of the strip 200 and the inner member 150 of the balloon 100. As the portion 130 of the lobe 120 expands, the portion 130 can apply a force or torque on the strip 200. The pressure of the expansion of the lobe 120 can overcome the rigidity and resistance to twist of the strip 200, thereby rotating the strip 200. The rigidity of the strip 200 can keep the strip 200 in a transverse orientation during delivery. The pressure of the lobes 120 must overcome this rigidity to rotate the strip 200. The expansion of the lobes 120 can cause the rotation of the strip 200 to occur. The directionality of the rotation of the strips 200 can be influenced by the pleating. The pleating can direct the expansion of the lobes 120. The controlled expansion of the lobes 200, in relation to the shaped side walls and rigidity of the strips 200, can cause reliably and predicable rotation. The rotation can be bi-directional. The strips 200 can rotate one direction with inflation and rotate the other direction with deflation. The strips 200 can reversibly change between configurations. The shape of the lobes 120 can be load centering. The shape of the lobes 120 can ensure stability when rotating the strips 200. The shape of the lobes 120 can prevent tipping of the strips 200. The lobes 120 can allow the strips 200 with the raised features 300 to rotate to a perpendicular orientation. The lobes 120 can allow the strips 200 with the raised features 300 to repeatedly return to the perpendicular orientation. The lobes 120 can allow the strips 200 with the raised features 300 to repeatedly return to the transverse orientation.

In some embodiments, the lobes 120 can apply a force to the sloped side walls of the strip 200 and the raised features 300 to rotate the strip 200 and the raised features 300 from the generally perpendicular orientation to the generally tangential orientation. In some embodiments, the balloon 100 with the strip 200 and the raised features 300 can be bi-directional to allow the plurality of strips 200 to rotate clockwise or counterclockwise. In some embodiments, the plurality of strips 200 point counterclockwise in the generally tangential orientation before inflation. In some embodiments, the plurality of strips 200 point counterclockwise in the generally tangential orientation after inflation. In some embodiments, the plurality of strips 200 can be at least partially covered by a pleat of the balloon 100 in the generally tangential orientation. In some embodiments, each strip 200 can be at least partially covered by a pleat of the balloon 100 when the balloon 100 is deflated. In some embodiments, the covered strips 200 can be located near the base of the pleated wings of the balloon 100. In some embodiments, the covered strips 200 can be oriented with the radially outward facing surface 304 laying tangential to the circumference of the inner member 150 limiting the radially outward facing surface 304 from contacting the pleated wings of the balloon 100. In some embodiments, the sloped side walls of the strip 200 and the raised features 300 in combination with the expansion of the lobes 120 can be configured to allow for more effective control of the generally perpendicular orientation of the strip 200 and the raised features 300.

During one or more of the above events described herein, the pressure wave can be generated. During one or more of the above events described herein, the balloon 100 can be pulsated by the pressure wave. The balloon 100 can be at least partially inflated while the pressure in the balloon increases by the pressure wave. The balloon 100 can be pulsated by the pressure wave during inflation in some methods. The balloon 100 can be pulsated by the pressure wave during the initial expansion period in some methods. The balloon 100 can be inflated without being pulsated by the pressure wave in some methods. The balloon 100 can be pulsated by the pressure wave after the initial expansion period in some methods. The balloon 100 can unfold in the vessel 10 and the strips 200 can be exposed from their resting place within the folds. The balloon 100 can be pulsated by the pressure wave during unfolding of the strips 200 in some methods. The balloon 100 can be unfolded without being pulsated by the pressure wave in some methods. The pressure wave can be generated when the balloon 100 is at least partially inflated. The pressure wave can be generated before the balloon 100 is in contact with the vessel 10. The pressure wave can be generated when the balloon 100 contacts the vessel 10. The pressure wave can be generated when the balloon 100 is unfolded. The pressure wave can be generated when the balloon 100 is initially expanding. The pressure wave can be generated when the balloon 100 is inflated greater than 0 ATM, 0.5 ATM, 1 ATM, 1.5 ATM, 2 ATM, 2.5 ATM, 3 ATM, or any range of two of the foregoing values. The pressure wave can accelerate unfolding. The pressure wave can accelerate rotation of the strip 200 from a tangential orientation to a perpendicular orientation. The pressure wave can accelerate formation of lobes 120 of the balloon 100. The pressure wave can accelerate the proper orientation of the raised feature relative to the vessel 10.

Continued Expansion Period without Penetration

FIG. 58C illustrates the continued expansion event with fluid filling the majority of the balloon 100 and expanding the balloon structure 100 to contact nearly all of the diseased vessel 10. The inflation device 400 can supply fluid to the reservoir 854 which can fill the balloon 100. The inflation device 400 can supply fluid at low pressures during the initial expansion event, such as a pressure of 0.5 ATM, 1 ATM, 1.5 ATM, 2 ATM, 2.5 ATM, 3 ATM, under 0.5 ATM, under 1 ATM, under 1.5 ATM, under 2 ATM, under 2.5 ATM, under 3 ATM, at least 0.5 ATM, at least 1 ATM, at least 1.5 ATM, at least 2 ATM, at least 2.5 ATM, at least 3 ATM, or any range of two of the foregoing values. During this phase of the expansion, the pressure can be still low (below 2 atmospheres) but enough to force the strips 200 with the raised features 300 into a perpendicular orientation. Note the vessel 10 illustrating an occlusion with a 2 mm inner diameter is shown below the expanded balloon surface 100. The balloon 100 in this phase can be behaving like that of plain balloons with minimal serration effects occurring. The rigidness of the strips 200 and the tightly bound disease can limit the strips 200 from penetrating the wall. The balloon 100 can be able to push the diseased vessel 10 outward while the lobes 120 of the expanding balloon 100 between the strips 200 continue to align the strip 200 with the raised features 300 upward and perpendicular to the vessel wall.

The balloon 100 can exert a pressure on the vessel 10 . . . . The pressure in the balloon 100 can be low, for instance such as under 2 atm. The diameter of the vessel can expand, in some methods. The expanded diameter can be slight. The balloon 100 can expand the vessel 10 to a larger diameter vessel 10′. The circles illustrate the diameter of the vessel 10 from FIG. 58B and the expanded diameter of the vessel 10′ are illustrated. The diameter of the vessel 10 can be 2 mm and the increased diameter of the vessel 10′ can be greater than 2 mm. The balloon 100 in this phase can exert a pressure on the vessel 10′ based on the pressure of the fluid within the balloon 100. The inflation device 400 can supply fluid to the balloon 100. The balloon 100 can continue to expand. The fluid can fill the majority of the balloon 100. The lobes 120 can continue to expand. The surface area of the lobes 120 in contact with the vessel wall can increase. The lobes 120 can contact nearly all of the vessel 10′. The lobes 120 can contact the vessel 10′ except for the area near the strips 200. The lobes 120 contact nearly the entire circumference of the vessel 10′.

The pressure in the balloon 100 can be low, for instance, less than 0.5 atm, less than 1 atm, less than 2 atm. The pressure in the balloon 100 can push the strips 200 to the desired orientation. The pressure can rotate the strips 200 with the raised features 300 to the perpendicular orientation. The fluid can fill areas of less resistance. The fluid can fill the lobes 120 of the balloon 100. The lobes 120 can act on the strips 200, thereby rotating the strips 200 to the perpendicular orientation.

While not to be limited by a theory, the portion of the lobe 130 can be fully expanded against the sides of the strip 200. The portion 130 can expand from a location between the surface of the strip 200 and the inner member 150 of the balloon 100 to a location beside the inner facing surfaces of neighboring strips 200. The portion 130 can expand to be against the strip 200. The portion 130 can apply a force or torque on the strip 200 to orient the strip 200 with the raised features 300 perpendicularly. Two adjacent lobes 120 can apply a force to the strip 200. The two adjacent lobes 120 can apply a pressure to orient the strip 200 with the raised features 300 perpendicularly. During this phase of the expansion, the pressure of the fluid can be under 2 atm. The pressure can be sufficient to rotate the strip 200. The strip 200 with the raised features 300 can rotate from the transverse orientation shown in FIG. 58A to the perpendicular orientation in FIG. 58C. The balloon 100 can include one or more intermediate orientations as the balloon 100 is inflated and the strip 200 rotates.

In some embodiments, referring to FIG. 13, the side surfaces 204 the strips 200 can be sloped, with the base wider than the top. The side surfaces 204 can be flat. In some embodiments, referring to FIG. 13, the anterior surface 310 and the posterior surface 312 of the raised features 300 can be sloped, with the base wider than the top. FIG. 14 schematically illustrates an example of the anterior surface 310 of the raised feature 300. The raised features 300 can include strip-facing surface width W_{B} which slopes to the radially outwardly facing surface width W_{U}. There can be an angle that can be equal to or less than about 90 degrees that defines the slope from the strip-facing surface width W_{B} to the radially outwardly facing surface width W_{U}. There can be a constant slope angle or a plurality of different angles, such as more than a single slope angle such as a double, triple or more bevel. In some embodiments, there can be a series of steps at different heights where the width transitions to a narrower width toward the radially outwardly facing surface 304. The anterior surface 310 and the posterior surface 312 of the raised features 300 can be flat. The strip 200 with the raised features 300 together can have a sloped side wall. The sloped side walls of the strip 200 with the raised features 300 can be adjacent to the lobes 120. The lobes 120 apply a pressure to the sloped side walls of the strip 200 with the raised features 300. The lobes 120 apply a pressure to the sloped side walls to rotate the strips 200 during inflation, as described herein. This slope of the side walls, in combination with the expansion of the portion 130 of the lobes 120 adjacent to the strips 200, can allow for more effective control of the perpendicular orientation of the strips 200 with the raised features 300. The lobes 120 can apply a pressure to the sloped side walls to maintain the perpendicular orientation during inflation, as described herein. The lobes 120 can apply pressure to the side surfaces 204 of the strip 200. The lobes 120 can also apply pressure to the side surfaces 310, 312 of the raised features 300. The lobes 120 can bulge out around the profile the strip 200 with the raised features 300. The lobes 120 can bulge out above the with the raised features 300. With the lobes 120 bulging out in the regions above the strip 200 with the raised features 300, the base of the strip 200 can be more effectively positioned into the optimized perpendicular orientation. The sloped side walls can be considered another element that enable the strip 200 with the raised features 300 to be perpendicular. The lobes 120 can expand in and around the raised features 300 of the strip 200 to help maintain the perpendicular orientation. In some embodiments, the two side walls of a single strip 200 with raised features 300 can have the same slope. The side walls can experience the same loads from adjacent lobes 120. The lobes 120 can reach an equilibrium based on the sloped side walls of the strips with raised features 300. As the lobes 120 extend up the sloped side walls, the lobes 120 can steady the strip with raised features 300 in a perpendicular orientation.

The pressure can be sufficient to slightly expand the vessel from 10 to 10′ as shown in FIG. 58C. The expanded vessel 10′ can allow the strips 200 to have the perpendicular orientation. The strips 200 can be positioned inward from the vessel 10′. In some methods, the radially outwardly facing surface 304 of the raised feature 300 does not contact the vessel 10′. In some methods, there can be no serration effect or minimal serration effect in the position of the strips 200 with raised features 300 shown in FIG. 58C. In this position, the radially outwardly facing surface 304 may not touch the vessel 10′. The rigidness of the strips 200 along with the hardness of the disease can limit or prevent penetration into the vessel 10′. The vessel can allow for slight expansion under low pressure from vessel 10 to vessel 10′. The vessel 10′ can be tightly bound. The vessel 10′ can be calcified or diseased, thereby limiting the penetration of the strip 200 with raised features 300. The balloon 100 can be able to slightly expand the diseased vessel outward under low pressure. The vessel can expand to vessel 10′. The lobes 120 of the balloon 100 can push against the vessel 10 to expand the vessel 10′. The lobes 120 of the balloon 100 can continue to align the strips 200 with raised features 300 between the lobes 120. The lobes 120 of the balloon 100 can continue to align the strips 200 radially outwardly facing surface 304 perpendicularly. The lobes 120 of the balloon 100 can continue to align the strips 200 radially outward from the inner member 150 of the balloon 100.

The strips 200 can be attached to an outer surface of the balloon 100 as described herein. The strips 200 can have one or more layers overlapping the strips 200. The outer retention balloon 104 can overlie a portion of the strips 200. The raised features 300 can rise above the outer retention balloon 104. The strips 200 can be bonded to the base balloon 102. The strips 200 can be bonded to the surface of the balloon 100. In some embodiments, the strips 200 may not be contained in a rigid structure. The strips 200 can be unattached from adjacent strips 200. The strips 200 can be unconstrained from a carrier. The strips 200 with the raised features 300 can be free to rotate as the balloon 100 expands. The strips 200 with the raised features 300 can be delivered in a flattened, rotated state. The strips 200 with the raised features 300 can undergo rotation as the balloon 100 expands. The strips 200 with the raised features 300 can undergo rotation as the balloon 100 deflates. The strips 200 with the raised features 300 can be considered self-righting. The strips 200 with the raised features 300 can be considered self-laying down. The strips 200 with the raised features 300 can rotate to the proper orientation during use. The strips 200 with the raised features 300 can rotate to the proper orientation during deflation. The lobes 120 can provide repeatable and predicable orientation of the strips 200 with the raised features 300.

During one or more of the above events described herein, the pressure wave can be generated. During one or more of the above events, the balloon 100 can be pulsated by the pressure wave. The balloon 100 can be inflated during the continued expansion period while the pressure in the balloon 100 increases. The balloon 100 can be inflated during the continued expansion period before the raised features 300 penetrate the vessel 10. The balloon 100 can be pulsated by the pressure wave during the continued expansion period in some methods. The balloon 100 can be inflated without being pulsated during the continued expansion period in some methods. The balloon 100 can be pulsated by the pressure wave to hammer against the vessel 10. The balloon 100 can be pulsated by the pressure wave to expand the diameter of the vessel 10 to vessel 10′. The raised features 300 of the strips 200 contact the wall of the vessel during the continued expansion period without penetration. In some methods, the radially outward facing surfaces 304 of the raised features 300 of the strips 200 do not contact the wall of the vessel 10 during the continued expansion period without penetration. In some methods, the radially outward facing surfaces 304 of the raised features 300 of the strips 200 contact the wall of the vessel 10 but do not penetrate the wall of the vessel 10 during the continued expansion period. The balloon 100 can be pulsated with the pressure waves while the raised features 300 of the strips 200 contact the wall in some methods. The raised features 300 of the strips 200 can contact the wall without the balloon 100 being pulsated by the pressure wave in some methods. In some methods, the raised features 300 of the strips 200 do not penetrate the wall generating nucleation sites for the fissuring event during the continued expansion period without penetration. The pressure wave can be generated when the balloon is in contact with the vessel 10. The pressure wave can be generated when the balloon is in contact with the expanded vessel 10′. The pressure wave can be generated to expand the vessel 10 to vessel 10′. The pressure wave can be generated before the strip 200 contacts the vessel. The pressure wave can be generated before the raised features 300 contact the vessel. The pressure wave can be generated when the raised features 300 are not yet contacting the vessel. The pressure wave can be generated before penetration. The pressure wave can be generated prior to nodes of weakness at points of contact of the strips 200 to the calcium sheet. The pressure wave can be generated before creating of microperforations. The pressure wave can be generated when the lobes 120 oppose the vessel. The pressure wave can be generated when the balloon 100 is inflated. The pressure wave can be generated when the balloon 100 is inflated to 0.5 ATM, 1 ATM, 1.5 ATM, 2 ATM, 2.5 ATM, 3 ATM, or any range of two of the foregoing values. The pressure wave can be generated when the balloon 100 is inflated to base pressure. The pressure wave can extend through the balloon 100. The pressure wave can promote the strip 200 to raise and penetrate the internal elastic lamina. The pressure wave can promote the strip 200 to raise and impact the calcium with greater impact force. The increase in the impact force of the strip 200 can increase the likelihood of the strip 200 to weaken the calcium. The weaken calcium promotes a zone for ultrasonic energy to propagate through the calcium generating higher regional sheer forces in the calcium. The sheering forces enable faster cracking propagation with regions of lower energy states within the calcium sheet. The fracture planes promoted by the strip 200 increase the balloon 100 to delivery ultrasonic energy to the weakened calcium promoting lobe generated ultrasound delivery to be more effective at crack promotion. Through contact impact and contact delivery of ultrasound, both regionally at the contact points and more globally at the lobe regions, a lower amplitude of ultrasound is needed to produce effective and safe calcium fracturing through out the diseased morphology. The balloon 100 can be contact with the majority of the circumference of the vessel. The pressure wave can pass through the lobes 120 of the balloon 100. The pressure waves can cause calcium fracturing. The pressure waves can create a shear stress on the calcium sheet. The pressure wave can provide a lithotripsy effect before penetration. The pressure wave can accelerate rotation of the strip 200 from a tangential orientation to a perpendicular orientation. The strip 200 can be properly oriented perpendicular to the balloon quicker with pressure waves than without pressure waves. The pressure wave can accelerate the application of tensile force by the lobes 120. The pressure wave can accelerate the expansion of the vessel 10.

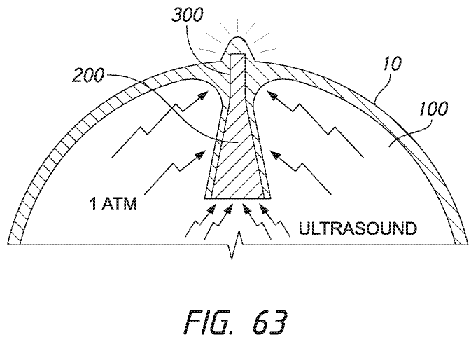

While not to be limited by a theory, the pressure wave can be transmitted through the balloon before penetration. FIG. 63 illustrates an example of how the pressure wave can be transmitted through the balloon 100 to the strip 200 with the raised features 300 during the inflation cycle. At this interim stage of balloon inflation, when the raised feature 300 encounters a rigid calcium structure, the acoustic wave energy can pass through the walls of the lobes 120 and the base of the strip 200 into the strip 200. The collection of the acoustic wave energy in the strip 200 can be highest at this interim point. This increase in energy can more effectively pass through the raised features 300 and into the hardened plaque. The balloon 100 can be compliant. The lobes 120 of the balloon can press against the side walls of the strip 200 with the raised feature. The balloon 100 can conform to the strip 200 with the raised features 300. The pressure wave can be generated. The lobes 120 can direct the pressure wave toward the raised feature 300. The base of the strip 200 can direct the pressure wave toward the raised feature 300. The energy of the pressure wave can be concentrated to the raised feature. The raised feature 300 can deliver this energy to create the serration. The raised feature 300 can deliver this energy to propagate the crack. The raised feature 300 can deliver this energy to create cavitations.

Continued Expansion Period with Penetration

FIG. 58D illustrates the continued expansion event with pressure of the balloon at atmospheres of between 2 atm and 4 atm in some embodiments. The inflation device 400 can supply fluid to the reservoir 854 which can fill the balloon 100. The inflation device 400 can supply fluid at higher pressures during the continued expansion event, such as a pressure of 2 ATM, 2.5 ATM, 3 ATM, 3.5 ATM, 4 ATM, 4.5 ATM, 5 ATM, 5.5 ATM, 6 ATM, under 2 ATM, under 2.5 ATM, under 3 ATM, under 3.5 ATM, under 4 ATM, under 4.5 ATM, under 5 ATM, under 5.5 ATM, under 6 ATM, at least 2 ATM, at least 2.5 ATM, at least 3 ATM, at least 3.5 ATM, at least 4 ATM, at least 4.5 ATM, at least 5 ATM, at least 5.5 ATM, at least 6 ATM, or any range of two of the foregoing values. During this phase of the expansion, the pressure exerts a greater force at the base of the strips 200 with the raised features 300 while still aiding the strips 200 to maintain their perpendicular orientation. Note the vessel 10′ is a circle illustrating a penetration into tissue with a 3 mm inner diameter. The balloon 100 in this phase can be producing serrated lines through the intimal tissue and into the medial plane. At this phase, the physician can hold the balloon 100 for a period of time to allow the serrated lines to seat and initiate the serration effect as described herein. The period of time can be 10 seconds, 20 seconds, 30 seconds, 40 seconds, 50 seconds, 60 seconds, less than 10 seconds, less than 20 seconds, less than 30 seconds, less than 40 seconds, less than 50 seconds, less than 60 seconds, greater than 10 seconds, greater than 20 seconds, greater than 30 seconds, greater than seconds, greater than 50 seconds, greater than 60 seconds, or any range of two of the foregoing values.

With the raised features 300 on the strips 200 being pushed into the vessel 10′, a series of lines of weakness can be formed along the serrated lines. The serrated lines can have spaces between the microperforations. In some embodiments, the serrated lines may not be continuous. The serrated lines can barely disrupt the diseased vessel 10′. The lobes 120 can exert a pressure on the vessel 10′. The lobes 120 can be in contact with a significant majority of the circumference of the vessel 10′. The lobes 120 can push on the vessel wall 10′. As the lobes 120 expand the vessel 10′, the lobes 120 can generate a tensile force under the regions where the raised features 300 have penetrated the tissue. This stretching of the vessel 10′ by the lobes 120 enables the vessel lumen to expand. A linear dissection line can be produced as the expansion of the diseased vessel 10′ increases. The lumen gain can be with minimal arterial injury. The weakened tissue can more easily give to the pressure of the lobes 120 of the balloon 100 and the weakened tissue can yield. The balloon 100 can be more easily able to push the diseased vessel 10′ outward, the raised features 300 of the strips 200 can form serrations which slowly combine into a linear dissected line pulling away from the raised features 300 of the strips 200 and gently expanding the diameter of the vessel 10′. The lobes 120 can be pushing the vessel 10′, thereby creating a tensile force. Under the stretching of the vessel 10′ due to the lobes 120, the permanent linear dissection line can be created. Under the stretching of the vessel 10′ due to the lobes 120, there can be permanent lumen gain. Under the stretching of the vessel 10′ due to the lobes 120, the low pressure force can prevent or reduce vessel injury. The lobes 120 can stretch the vessel 10′ to cause the vessel 10′ to open and the permanent linear dissection line to be created. The tearing along the microperforations created by the raised features 300 can be significantly caused by the lobes 120. The lobes 120 can exert a tensile force to facilitate the tearing.

The balloon 100 continues to fill under increased pressure. The pressure inside the lobes 120 rises. While the lobes 120 were around 2 atm or less in the previous phase, the pressure increases. The pressure can increase gradually from 2 atm to up to 4 atm, for instance up to 2 atm, at least 2 atm, 2.5 atm, up to 2.5 atm, at least 2.5 atm, not more than 2.5 atm, 3 atm, up to 3 atm, at least 3 atm, not more than 3 atm, 3.5 atm, up to 3.5 atm, at least 3.5 atm, not more than 3.5 atm, 4 atm, up to 4 atm, at least 4 atm, not more than 4 atm, or any range of two of the foregoing values. The lobes 120 can be fully expanded against the surface of the vessel wall 10′. The vessel 10′ can be diseased, thereby resisting further expansion of the vessel wall 10′. The vessel 10′ can be diseased, thereby allowing minimal expansion from the lobes 120 of the vessel wall 10′ of less than 0.5 mm, less than 1.0 mm, less than 1.5 mm, less than 2.0 mm of any range of two or more of the foregoing values. The balloon 100 can cause minimal dissection. The balloon 100 can expand the vessel 10′ very slightly. The serrated lines, acting under the tensile force of the lobes 120, can cause a bigger growth in the diameter of the vessel 10′. The permanent linear dissection line which can occur later in the procedure can allow for increased lumen gain of the vessel 10′. In some embodiments, the expansion of the balloon 100 under low pressure at this stage can do little to expand the vessel 10′, since the permanent linear dissection line have not been formed.

The fluid can fill the balloon 100 in areas with least resistance. There can be a portion of the balloon 100 under the strip 200. This portion 140 of the balloon 100 can be inflated after the lobes 120, in some methods. The lobes 120 can present less resistance to filling, in some situations. There can be a balance between which areas of the balloon 100 exert a greater resistance to flow. In some instances, the portion 140 can present less of resistance than further expansion of the vessel wall 10′. The portion 140 can be inflated under increased pressure from the fluid. The increased pressure can overcome the inertial force or rigidity of the strip 200 to initiate the node of penetration by the raised feature 300 into the intimal tissues of the vessel wall. The pressure of the fluid of the balloon 100 can exert a greater force on the strip 200. The pressure of the balloon 100 can exert a force on the base of the strips 200. The pressure of the balloon 100 can exert a force on the base of the strips 200 after the strips 200 with the raised features 300 are in their perpendicular orientation.

The strip 200 with the raised features 300 can gradually push outward. The strips 200 with the raised features 300 can penetrate the intimal tissue. The strip 200 with the raised features 300 can expand radially outward relative to the inner member 150 of the balloon 100. The strips 200 with the raised features 300 can penetrate the internal elastic lamina. The strips 200 with the raised features 300 can be disposed between the lobes 120. The lobes 120 can facilitate the perpendicular orientation. The strips 200 with the raised features 300 can maintain their orientation relative to the lobes 120 during expansion. The lobes 120 can guide the strips 200 with the raised features 300 during expansion. The strip 200 with the raised features 300 can move radially outward. The strip 200 with the raised features 300 can move radially outward from the inner member 150 of the balloon 100. The portion 140 of the balloon 100 under the base of the strip 200 can continue to expand.

In some methods, the vessel 10′ may not expand during this phase. The vessel 10′ may not expand at these low pressures, for instance under 4 atm. The low pressure may not be sufficient to cause further expansion of the diameter of the vessel 10′. On balance, the area of least resistance can be filling the portion 140 at the base of the strip 200 compared with further expanding the vessel 10′.

While not to be bound by a theory, the area 140 under the strip 200 can be able to expand due to many considerations. The area 140 can be small relative to the lobes 120 that have already been filled. The geometry of the area 140 of the balloon 100 under the base of the strips 200 can facilitate movement of the strip 200. The strip 200 can have a thin base. Referring back to FIG. 13, the base width of the strip 200 can be many factors smaller than the base length of the strip 200. The area 140 of the balloon corresponds to the surface area of the base.

While not to be bound by a theory, the area 140 can act to generate a force to move the strip 200 when pressure inside the balloon 100 is increased. The area 140 can be thin and long compared with the lobes 120. The pressure in the area 140 under the strip 200 can cause movement outward of the strip 200 as the pressure rises. The increase pressure of the fluid must go somewhere and the area of least resistance can be the area 140 of the balloon 100 under the base of the strip 200. There can be many factors that contribute to the outward movement of the strips 200 with the raised feature 300. The lobes 120 can be filled and act along the sloped sides of the strips 200 with the raised feature 300 to maintain the perpendicular direction. The forces of the balloon 100 can provide directional guidance for the outward movement of the strips 200 with the raised feature 300. There can be a pressure change from the mechanics of the vessel 10′. The reason for the change in pressure distribution can be due to the raised features 300 of the strip 200 having induced nodes of separations in the intima. These nodes of separation allow the raised features 300 of the strip 200 to have a place to go. There can be low pressure from the vessel 10′ pushing back on the strip 200 with raised features 300. This low pressure from the vessel 10′ in combination with the pressure from the lobes 120 of the balloon 100 can allow the strips 200 with raised features 300 to move radially outward, further into the vessel 10′. The strips 200 with raised features 300 can rapidly accelerate toward the medial layer when the initial resistance from the vessel 10′ is overcome by node formation. The low pressure of the balloon 100, such as pressures under 4 atm, can be sufficient to drive the raised features 300 of the strip 200 into the vessel 10′ after node formation. The interaction between the strip 200 and the vessel 10′ can be dynamic, changing over time under sustained pressure from the balloon 100. The design and method of the balloon 100 can encourage the raised features 300 to penetrate under the increased pressure on the vessel 10′ by the lobe 120. The strips 200 with raised features 300 can extend into the tissue at low pressure. The balloon 100 can create a strain under the strip 200 with raised features 300 to encourage outward movement.

The strip 200 with raised features 300 can be pliable, yet durable and rigid. While not to be bound by a theory, the fluid pressure within the balloon 100 must overcome the rigidity of the strip 200 with raised features 300 to push the strip 200 with raised features 300 radially outward into diseased tissue. In some methods, the pressure must overcome the inertia of the static strip 200 with raised features 300 to penetrate the disease. In some methods, the pressure can be 4 atm or less. The strip 200 with raised features 300 can be configured to move radially outward at this low pressure.

The vessel 10′ is illustrated. The vessel 10′ can have any diameter. The vessel 10′ can have a greater cross-sectional dimension than the initial vessel 10. The vessel 10′ can be slightly expanded under the low pressure of the balloon 100. The pressure can be less than 4 atm. This low pressure can be insufficient to cause significant dilation of the vessel 10′. In some methods, the vessel 10 can be expanded 0.25 mm, 0.50 mm, 0.75 mm, 1 mm, or any range of two of the foregoing values. In some methods, the vessel wall 10 can be expanded 10%, 20%, 30%, 40%, 50%, or any range of two of the foregoing values. The vessel wall 10 can be slightly expanded to 10′ due to the low pressure of the fluid of the balloon 100, wherein the pressure can be 4 atm or less. The strips 200 with raised features 300 can penetrate the intima. The strips 200 with raised features 300 can penetrate the internal elastic lamina. The strips 200 with raised features 300 can penetrate into the medial tissue.

The strip 200 with raised features 300 can radially expand to the vessel wall 10′. The raised feature 300 of the strip 200 can include the radially outwardly facing surface 304 which can be unhoned. The top view unhoned surface can be slightly blunt. The unhoned surface can have a width. The width of the unhoned surface can be less than the width of the base but can be directly correlated with the slope or slopes of the raised feature 300. The unhoned surface can be any geometry described herein. In some embodiments, the radially outward facing surface 304 can be rectangular, square, oval, rounded, polygonal, or any other shape having a non-zero width. In some embodiments, the radially outward facing surface 304 can have a width, for example, that can be about 20 nm, 50 nm, 100 nm, 500 nm, 1 μm, 2 μm, 5 μm, 7 μm, 10 μm, 15 μm, 20 μm, 25 μm, and 30 μm, 40 μm, 50 μm, 60 μm, 70 μm, 80 μm, 90 μm, 100 μm, between 5 μm and 30 μm, greater than 30 μm, between 30 μm and 100 μm, or any range of two of the foregoing values, measured at the radially outward facing edge or surface. In some embodiments, the radially outward facing surface 304 does not include a sharp honed point or edge as described herein. The radially outward facing surface 304 can have a width that can be larger than that of a honed edge. The raised feature 300 can have the radially outward facing surface 304 that can be completely unhoned along its length. The raised feature 300 of the strip 200 can have any feature described herein.

The radially outward facing surface 304 of the raised feature 300 of the strip 200 can contact the vessel wall 10′. While not to be bound by a theory, the radially outward facing surface 304 of the raised feature 300 of the strip 200 does not function as a blade. In some embodiments, the radially outward facing surface 304 of the raised feature 300 of the strip 200 may not be a honed cutting surface. The radially outward facing surface 304 of the raised feature 300 of the strip 200 is not sharpened. With honed edges such as blades, the edge can apply high pressure with minimal force applied to push the material part. The honed edge can cut through the material from the extremely narrow edge. The effectiveness of the honed edge can relate to the sharpness or geometry of the edge. The honed edge can separate molecules, such as molecules of a vessel wall. The honed edge of the raised features 300 can push aside longitudinally oriented intima cells. The honed edge of the raised features 300 can minimize injury to cells. The molecules can slip past each other at the honed edge. The narrower in width the edge for honed edges, e.g., the closest it comes to atomic-scale sharpness, the easier the edge can cut through material.

The radially outward facing surfaces 304 of the raised features 300 can be advantageously unhoned. The radially outward facing surfaces 304 of the raised features 300 can be advantageously slightly blunt. The raised features 300 of the strip 200 can create serrations, indentations, and/or microperforations in the vessel 10′. These serrations, indentations, and/or microperforations in the vessel 10′ can be desirable rather than making cuts through the vessel 10′, such as with a honed surface. While not to be bound by a theory, the serrations, indentations, and/or microperforations in the vessel 10′ can occur under the influence of many forces. The radially outward facing surface 304 of the raised feature 300 of the strip 200 can be against the vessel 10′. The fluid pressure of the balloon can apply a force to firmly contact the raised feature 300 of the strip 200 with the vessel wall 300′. In some instances, the width of the radially outward facing surface 304 of the raised 300 can be too great to penetrate the vessel 10′ under the influence of the fluid pressure of the balloon 100. The pressure can be 4 atm or less. In some instances, the radially outward facing surface 304 of the raised 300 can be unable to penetrate into the vessel 10′ under pressure at area 140 alone.

The serrations, indentations, and/or microperforations can occur due to, at least in part, the force exert by the lobes 120. While not to be bound by a theory, the lobes 120 exert a force on the vessel 10′ causing the vessel 10′ to pull away from the raised features 300 of the strip 200. In some instances, the vessel 10′ does not pull apart at the radially outward facing surfaces 304 of the raised features 300. Rather, the vessel 10′ can pull apart along the height of the raised features 300. The mechanics of the expansion of the balloon 100 can facilitate the serration by the raised features 300. The mechanics of the expansion of the balloon 100 can allow serrations to form at low pressure, wherein the raised features 300 was unable to penetrate due to pressure on the strip 200 alone.

As described herein, the strip 200 can include a plurality of raised features 300 spaced apart along a surface of each strip 200. In some embodiments, the strip 200 does not comprise a longitudinally continuous penetration surface. Rather, the strip can include discontinuities or spaces between the raised features 300. The vessel 10′ can pull away from the strip 200 only at or near the raised features 300. The vessel 10′ can remain intact at or the discontinuities or spaces between the raised features 300. The strip 200 with the raised features 300 and the balloon 100 thereby create serrations, indentations, and/or microperforations. The serrations, indentations, and/or microperforations can be discontinuous. The serrations, indentations, and/or microperforations pattern can mirror the pattern of the raised features 300 of the strips 200. Due to crack propagation, the serrations, indentations, and/or microperforations can be slightly larger than the width and length of each raised feature 300. The vessel 10′ pulls way from plurality of raised features 300 of the strip 200 thereby creating serrations, indentations, and/or microperforations. The serrations, indentations, and/or microperforations can be longitudinally oriented. The serrations, indentations, and/or microperforations can be discrete. The serrations, indentations, and/or microperforations can be discontinuous.

The mechanics of creating serrations, indentations, and/or microperforations can be a highly complex process. The geometry of the strip 200 with the raised features 300 can impact the geometry of the resulting serrations, indentations, and/or microperforations. The pressure exerted by the balloon 100 at the lobes 120 can impact the geometry of the resulting serrations, indentations, and/or microperforations. The pressure exerted by the balloon 100 at the area 140 under the base of the strips 200 can impact the geometry of the resulting serrations, indentations, and/or microperforations. The surface of the vessel 10′ can impact the geometry of the resulting serrations, indentations, and/or microperforations. While not to be bound by a theory, the serrations, indentations, and/or microperforations can be created by the 10′ separating at an atomic level along the height of the raised features 300 under the influence of pressure by the balloon 100 both on the vessel 10′ and on the strip 200 with the raised features 300.

The strip 200 with the raised features 300 can expand radially outward as serrations, indentations, and/or microperforations form in the vessel 10′. As the vessel 10′ opens, there can be less pressure preventing the outward expansion of the strip 200 with the raised features 300. In some methods, the balloon 100 does not have to disturb the entire lesion. Rather, the balloon 100 can disturbs a very small area near the raised features 300 to create serrations, indentations, and/or microperforations in the lesion. The strip 200 with the raised features 300 can penetrate into serrations, indentations, and/or microperforations formed in the lesion beyond the wall of the vessel 10′. The strip 200 with the raised features 300 can penetrate into serrations, indentations, and/or microperforations as the serrations, indentations, and/or microperforations are formed. As the vessel 10′ opens into serrations, indentations, and/or microperforations due to the mechanics of the balloon 100 and the strip 200 with the raised features 300, the area 140 of the balloon 100 under the strip can have less resistance to expansion. The serrations, indentations, and/or microperforations can expand deeper into the vessel 10′ under the influence of the pressure of the balloon 100. In some methods, the pressure of the balloon 100 can be 4 atm or less.

The vessel 10′ is illustrated in relation to the strip 200 with the raised features 300. The strip 200 with the raised features 300 can penetrate a distance beyond the wall of the vessel 10′ illustrated in the dashed circle. In some methods, the strips 200 with the raised features 300 can extend 0.25 mm, 0.50 mm, 0.75 mm, 1 mm, or any range of two of the foregoing values. In some methods, the strips 200 with the raised features 300 can be extend a percentage of the diameter of the vessel 10′ such as 10%, 20%, 30%, 40%, 50%, or any range of two of the foregoing values.

The balloon 100 and the strips 200 with the raised features 300 can produce a serrated line. The serrated lines can be discontinuous, at least initially. The serrated lines can be discrete perforations into the lesion and/or vessel 10′, at least initially. The serrated lines can mirror the spacing and separation of the raised features 300 on the strip 200, at least initially.

The serrated lines can be through the wall of the vessel 10′. The serrated lines can be through the intimal tissue of the vessel 10′. The serrated lines can be into the medial plane of the vessel 10′. While not to be bound by a theory, the serrations, indentations, and/or microperforations do not occur instantaneously. For a honed surface, the edge can instantly cut the surface of a vessel. For the complex interactions between the radially outward facing surfaces 304 of the raised features 300 of the strip 200 and the pressure of the balloon 100, the penetration may not be instantaneous. The pressure of the balloon 100 can cause a tearing along the surface height of the raised feature 300, in some methods. The pressure of the balloon 100 can cause slow outward movement of the strip 200 with the raised features 300 due to the pressure at the portion 140 under the strip 200. The serrations, indentations, and/or microperforations can slowly creep through the intimal tissue. The serrations, indentations, and/or microperforations can slowly creep through into the medial plane. In some methods, the creation of serrations, indentations, and/or microperforations can take a period of time. This period of time can be 5 seconds, at least 5 seconds, 10 seconds, at least 10 seconds, 20 seconds, at least 20 seconds, greater than 20 seconds, 30 seconds, at least 30 seconds, greater than 30 seconds, 40 seconds, at least 40 seconds, greater than 40 seconds, 50 seconds, at least 50 seconds, greater than 50 seconds, 60 seconds, at least 60 seconds, greater than 60 seconds, or any range of two of the foregoing values. The period of time can be sufficient to allow the strip 200 with the raised features 300 to seat within the intima layer. The period of time can be sufficient to allow the serrations, indentations, and/or microperforations to extend to intima layer. The period of time can be sufficient to allow the strip 200 with the raised features 300 to seat within the medial layer. The period of time can be sufficient to allow the serrations, indentations, and/or microperforations to extend to medial layer.

While not to be bound by a theory, the serrations, indentations, and/or microperforations can be described as initially being discrete and then merging. However, due to mechanics of the diseased vessel 10′, this may not occur in two stepwise phases. Rather, two or more serrations, indentations, and/or microperforations can merge into a line at any time. The serrations, indentations, and/or microperforations can merge at the intimal layer. The serrations, indentations, and/or microperforations can merge at the medial layer. The serrations, indentations, and/or microperforations can merge under the dynamics of crack propagation. Crack propagation can be a highly complex process based on the balances of energies. The crack can propagate between discrete serrations, indentations, and/or microperforations when the energy overcomes the resistance of the lesion or tissue. The crack can form between discrete serrations, indentations, and/or microperforations if the energy released by the growth of the crack is greater than the energy required to create the crack. The crack propagation can occur under the dynamic loading of the balloon 100. The crack propagation can occur under the influence of pressure of the balloon 100. The propagation can occur under the influence of time. The mechanics of the expansion of the balloon 100 facilitate subsequent crack propagation.

In some methods, the balloon 100 can be maintained at low pressure, such as 4 atm or less. The serrations, indentations, and/or microperforations can be formed. The strip 200 with the raised features 300 can be seated within the serrations, indentations, and/or microperforations. The strip 200 with the raised features 300 can reach an equilibrium. The strips 200 with the raised features 300 cannot expand further under the pressure of the balloon 100. The strip 200 with the raised features 300 can be held at this low pressure for a period of time. The propagation can be time dependent. The strip 200 with the raised features 300 can be held to allow a line of weakness or crack to be formed along the serrated line.

The strips 200 can be longitudinally oriented along the surface of the balloon 100. The strips 200 can be spaced along the surface of the balloon 100. The strips 200 can be angularly spaced along the surface of the balloon 100. The strips 200 can be 120 degrees apart along an arc length. The strips 200 can be spaced along a circumference. The strips 200 can be equidistant. The strips 200 can be circumferentially spaced such that serrated lines are sufficiently separated. The spacing between serrated lines can prevent or reduce circumferential crack propagation. The spacing between adjacent serrations, indentations, and/or microperforations in a longitudinal direction can be less than the spacing between adjacent serrations, indentations, and/or microperforations in a circumferential direction. The spacing between adjacent serrations, indentations, and/or microperforations in a circumferential direction can be a multiple, such as 2× greater, 5× greater, 10× greater, 15× greater, 20× greater, or any range of two of the foregoing values, of the spacing in a longitudinal direction.

The shape of the raised features 300 on the strip 200 can facilitate crack propagation. The radially outward facing surface 304 of the raised feature 300 can be rectangular. The radially outward facing surface 304 can have a greater length than width. The raised feature 300 can have discrete spaces therebetween along the strip 200. The serrations, indentations, and/or microperforations can mirror this shape of the raised feature 300 of the strip 200. The serrations, indentations, and/or microperforations can have a longer length than width. The serrations, indentations, and/or microperforations can have discrete spaces therebetween. While not to be bound by a theory, the serrations, indentations, and/or microperforations may more easily propagate in the longer direction, for instance along the length rather than along the width of the serrations, indentations, and/or microperforations.

The orientation of the strips 200 relative the balloon 100 can facilitate longitudinal crack propagation. The orientation of the strips 200 relative the balloon 100 can reduce or prevent circumferential crack propagation. The orientation of the strips 200 relative the balloon 100 can allow for repeatable and predictable crack formations. The orientation of the strips 200 relative the balloon 100 can produce lines of cracks along length of the balloon 100. The shape of the raised feature 300 on the strip 200 can dictate the direction of crack propagation. The shape of the serrations, indentations, and/or microperforations can dictate the direction of crack propagation.

While not to be bound by a theory, the serrations, indentations, and/or microperforations weaken the tissue. The weakened tissue can eventually give to the pressure of the balloon 100. The weakened tissue can give under the influence of time. The weakened tissue can allow the crack to propagate along the serrations, indentations, and/or microperforations. The serrations, indentations, and/or microperforations can slowly combine into a linear dissected line. The serrations, indentations, and/or microperforations can be initially formed by the raised features 300 of the strip 200. In some methods, the raised features 300 of the strip 200 does not act as a blade to form the linear dissected line. Rather, the linear dissected line can be formed only through crack propagation along the series of serrations, indentations, and/or microperforations, in some embodiments. The crack can propagate longitudinally. The balloon 100 can create one longitudinal crack, corresponding to one strip 200. The balloon 100 can create two longitudinal cracks, corresponding to two strips 200. The balloon 100 can create three longitudinal cracks, corresponding to three strips 200. The longitudinal cracks can allow the diameter of the vessel 10′ to be expanded outward. As the crack propagates, the balloon 100 may be able to more easily push the vessel 10′ outward to a larger diameter.

In some embodiments, the lobes 120 of the balloon 100 can be configured to exert a force on the vessel wall of the vessel 10′ that aids in crack propagation. The lobes 120 of the balloon 100 can exert a tensile force on the vessel wall of the vessel 10′ in the regions between the raised features 300. The lobes 120 of the balloon 100 can exert a force on nearly the entire circumference of the vessel wall of the vessel 10′. The lobes 120 of the balloon 100 can exert a force on a percentage of the circumference such as 50%, 55%, 60%, 65%, 70%, 75%, 80%, 85%, 90%, 95%, or any range of two of the foregoing values. In some embodiments, the lobes 120 can be configured to exert a force on the vessel wall of the vessel 10′ that allows the radially outward facing surface 304 to penetrate slightly to create nucleation sites for the fissure event. In some embodiments, the lobes 120 can be configured to exert a force on the vessel wall of the vessel 10′ that allows the radially outward facing surface 304 to penetrate slightly to create serrations in the vessel wall of the vessel 10′. In some embodiments, the lobes 120 can be configured to exert a force on the vessel wall of the vessel 10′ that allows the radially outward facing surface 304 to penetrate slightly into the intima. In some embodiments, the lobes 120 can be configured to exert a force on the vessel wall of the vessel 10′ for crack propagation. In some embodiments, the lobes 120 can be configured to exert a force on the vessel wall of the vessel 10′ causing the vessel wall to pull away from the raised features 300. In some embodiments, the lobes 120 can be configured to exert a force to connect the nucleation sites. In some embodiments, the lobes 120 can be configured to exert a force to create one longitudinally extending crack.

During one or more of the above events described herein, the pressure wave can be generated. During one or more of the above events, the balloon 100 can be pulsated by the pressure wave. The balloon 100 can be inflated during the continued expansion period while the pressure in the balloon 100 increases. The balloon 100 can be inflated during the continued expansion period with penetration. The balloon 100 can be pulsated with the pressure wave during the continued expansion period with penetration in some methods. The balloon 100 can be pulsated by the pressure wave to hammer against the vessel 10. The balloon 100 can be pulsated by the pressure wave to expand the diameter of the vessel 10 to vessel 10′. The raised features 300 of the strips 200 can contact the wall of the vessel. The tips or radially outward facing surfaces of the raised features 300 of the strips 200 penetrate the wall of the vessel 10 during the continued expansion period with penetration. The balloon 100 can be pulsated with the pressure wave while the raised features 300 of the strips 200 penetrate the wall of the vessel 10 in some methods. The raised features 300 of the strips 200 can penetrate the wall of the vessel 10 without the balloon 100 being pulsated in some methods. The raised features 300 of the strips 200 can penetrate the wall of the vessel 10 generating nucleation sites for the fissuring event. The relatively narrow profile of the raised features 300 of the strips 200 can penetrate the wall of the vessel 10. The raised features 300 can penetrate the intercellular matrix in the of the vessel 10. The raised features 300 can minimize cellular injury by limiting microscopic tearing. The balloon 100 can be pulsated by the pressure wave while the raised features 300 of the strips 200 penetrate the wall of the vessel 10 in some methods. The raised features 300 of the strips 200 can penetrate the wall of the vessel 10 without the balloon 100 being pulsated by the pressure wave in some methods. The balloon 100 can be pulsated by the pressure wave after the raised features 300 of the strips 200 penetrate the wall in some methods. The pressure wave can be generated when the raised features 300 and the balloon 100 are in contact with the vessel 10. The pressure wave can accelerate the formation of microperforations. The pressure wave can accelerate the raised features 300 for penetration. The pressure wave can accelerate the penetration of the internal elastic lamina. The pressure wave can accelerate the penetration into the calcium sheet. The pressure wave can accelerate the formation of stress lines. The pressure wave can accelerate the formation of multiple stress lines from each microperforation. The pressure wave can increase the number of stress lines from each microperforation. The pressure wave can increase the number of stress lines from each microperforation by 2×, 3×, 4×, 5×, 10×, 15×, 20×, or any range of two of the foregoing values. The pressure wave can cause shearing of the calcium sheet. The pressure wave can pass through the raised features 300 to shear the calcium sheet. The pressure wave can pass through the lobes 120 of the balloon 100 to shear the calcium sheet. The pressure wave can accelerate the raised features toward the vessel wall. The raised features 300 can penetrate faster with the pressure wave than without the pressure wave. The raised features 300 can penetrate with more force with the pressure wave than without the pressure wave. The raised features 300 can produce more shearing penetrate with more force with the pressure wave than without the pressure wave. The pressure wave can be generated after creating of microperforations. The pressure wave can be generated when the balloon 100 is inflated to 1 ATM, 1.5 ATM, 2 ATM, 2.5 ATM, 3 ATM, 3.5 ATM, 4 ATM, 4.5 ATM, 5 ATM, or any range of two of the foregoing values. The pressure wave can pass through the balloon 100. The balloon 100 can be contact with the majority of the circumference of the vessel. The pressure wave can pass through the lobes 120 of the balloon 100. The pressure waves can create a shear stress on the calcium sheet. The pressure wave can accelerate the application of tensile force by the lobes 120. The pressure wave can provide a lithotripsy effect during penetration. The pressure wave can provide a lithotripsy effect after penetration. The pressure wave can accelerate penetration of the raised features 300 through the internal elastic lamina. The raised features 300 can penetrate faster with pressure waves than without pressure waves. The raised features 300 can penetrate deeper with pressure waves than without pressure waves. The raised features 300 can create microperforations quicker with pressure waves than without pressure waves. The pressure wave can accelerate the expansion of the vessel 10 to the vessel 10′. The pressure wave can apply dynamic loading on the vessel 10. The vessel can expand under the load applied by the lobes 120 of the balloon when pressure waves are generated.

In some embodiments, the lobes 120 of the balloon 100 can be configured to exert a force to crush plaque. In some embodiments, the lobes 120 can be configured to exert a force to compress the plaque. In some embodiments, the lobes 120 can be configured to exert a mechanical force on the plaque. In some embodiments, the strip 200 with the raised features 300 can be configured to exert a mechanical force on the plaque. Lithotripsy can be a procedure that uses pressure waves to crush plaque. The plaque can have a crystalline structure. The pressure waves can cause the plaque to vibrate to crush the plaque. Some forms of lithotripsy use an ultrasonic means to fracture plaque. In some embodiments, the system 1100 described herein can utilize a mechanical means to form lithotripsy to crush plaque. In some embodiments, the lobes 120 of the balloon 100 can be configured to exert a tensile force on the plaque. In some embodiments, the lobes 120 of the balloon 100 can be configured to exert a tangential force on the plaque. In some embodiments, the lobes 120 of the balloon 100 can be configured to exert a longitudinal force on the plaque. In some embodiments, the pressure of the balloon 100 can exert a force on the plaque to crush the plaque and create the serrations. In some embodiments, the pressure of the balloon 100 and the raised features 300 in combination can crush the plaque. The system 1100 can effectively manage plaque by weakening the bonds of the plaque along serrated lines. The system 1100 can effectively manage plaque by initiating cellular or structural separation through crack propagation. The system 1100 can effectively manage plaque by structural compression or crushing of the plaque. The system 1100 can effectively manage plaque by avoiding plaque rupture by maintaining the mechanical strength of the majority of the plaque.

The generator 800 can be used to crush the plaque in the vasculature. The generator 800 can allow for the diseased lumen to be safely expanded and accurately dilated and stretched, using low pressure, to a desired diameter without creating dissections. The generator 800 can effectively crush the plaque, independent of plaque location. The generator 800 can effectively crush intimal plaque. The generator 800 can effectively crush medial plaque. The generator 800 can effectively crush a combination of both intimal and medial plaque. The generator 800 can effectively crush plaque through the combination of raised features 300 configured to penetrate the plaque and tension applied by the lobes 120 of the balloon 100 which in combination can fracture the plaque along designated lines. These fractured plaque elements can be more easily crushed through the subsequent balloon pressure of the balloon 100. The lobes 120 of the balloon 100 can exert a radial force to break the calcium bonds of the plaque. The lobes 120 of the balloon 100 can exert a radial force to facilitate crack propagation. The lobes 120 of the balloon 100 can exert a radial force on the plaque itself. The lobes 120 of the balloon 100 can exert a tangential force to break the calcium bonds of the plaque. The lobes 120 of the balloon 100 can exert a tangential force to facilitate crack propagation. The lobes 120 of the balloon 100 can exert a tangential force on the plaque itself. The balloon pressure of the balloon 100 can exert a longitudinal force to break the calcium bonds. The lobes 120 of the balloon 100 can exert a longitudinal force to facilitate crack propagation. The lobes 120 of the balloon 100 can exert a longitudinal force on the plaque itself. The lobes 120 of the balloon 100 can exert a radial, tangential, and longitudinal force on the plaque. These forces can exert a compounding force. These forces in combination with the serrated lines can induce energy upon the calcium bonds of the plaque. These forces cab break apart the calcium bonds therein enabling a crushing of the plaque into smaller calcium fragments. The smaller calcium fragments can be less restrictive to vasculature. With smaller vascular calcium fragments, the vasculature can return to a higher level of vascular compliance. The system 1100 can utilize balloon pressure in combination with a plurality of raised features 300 to individually impart radial, tangential, and longitudinal forces to fracture plaque calcification. The system 1100 can utilize balloon pressure in combination with a plurality of raised features 300 to align combined forces in longitudinal arrays to establish controlled fracture planes. The system 1100 can utilize balloon pressure in combination with a plurality of raised features 300 to result in vessel dilatation. The system 1100 can utilize balloon pressure in combination with a plurality of raised features to result in a vessel that can be higher in compliance. The native vessel can become more compliant post serration technology. The native vessel can be more compliant both during and after dilatation. The raised features 300 can be lithotripsy elements. The balloon 100 can have any features described herein. The strip 200 can have any feature described herein. The raised features 300 can have any feature described herein. The system 1100 can have any feature described herein.

### Fully Expanded Period

FIG. 58E illustrates the final resting balloon 100 in the fully expanded diameter typically at atmospheres of 4 or more in some embodiments. The inflation device 400 can supply fluid to the reservoir 854 which can fully expand the balloon 100. The inflation device 400 can supply fluid at higher pressures, such as a pressure of 4 ATM, 4.5 ATM, 5 ATM, 5.5 ATM, 6 ATM, 6.5 ATM, 7 ATM, 7.5 ATM, 8 ATM, 8.5 ATM, 9 ATM, 10 ATM, 11 ATM, 12 ATM, 13 ATM, 14 ATM, 15 ATM, under 4 ATM, under 4.5 ATM, under 5 ATM, under 5.5 ATM, under 6 ATM, under 6.5 ATM, under 7 ATM, under 7.5 ATM, under 8 ATM, under 8.5 ATM, under 9 ATM, under 10 ATM, under 11 ATM, under 12 ATM, under 13 ATM, under 14 ATM, under 15 ATM, at least 4 ATM, at least 4.5 ATM, at least 5 ATM, at least 5.5 ATM, at least 6 ATM, at least 6.5 ATM, at least 7 ATM, at least 7.5 ATM, at least 8 ATM, at least 8.5 ATM, at least 9 ATM, at least 10 ATM, at least 11 ATM, at least 12 ATM, at least 13 ATM, at least 14 ATM, at least 15 ATM, or any range of two of the foregoing values. During this phase of the expansion, the pressure of the balloon 100 exerts a greater force at the base of the strips 200 while still aiding the strips 200 with the raised features 300 to maintain their perpendicular orientation. The vessel 10′ can further expand to vessel 10″. The vessel 10″ is illustrated with a penetration into tissue by the raised features 300 beyond the vessel 10″. The vessel 10″ can have a 3 mm inner diameter. The balloon 100 in this phase can be producing serrated lines through the intimal tissue and into the medial plane. At this phase, the physician can hold the balloon 100 for a period of time, for instance 60 seconds or other times described herein, to allow the serrated lines to seat and initiate the serration effect as described herein. With the raised features 300 of the strips 200 being pushed into the vessel 10″, a series of lines of weakness can be formed along the serrated lines. The weakened tissue can more easily give to the pressure of the balloon 100 and the weakened tissue can yield. The balloon 100 can be more easily able to push the diseased vessel 10″ outward, the raised features 300 of the strips 200 form serrations which slowly combine into a linear dissected line pulling away from the raised features 300 of the strips 200 and gently expanding the diameter of the vessel 10″.

The balloon 100 can continue to fill. The pressure inside the lobes 120 can rise. While the lobes 120 can be around 4 atm or less in the previous phase, the pressure can increase in this phase. The pressure can increase gradually from 4 atm, for instance up to 4 atm, at least 4 atm, 4.5 atm, up to 4.5 atm, at least 4.5 atm, not more than 4.5 atm, 5 atm, up to 5 atm, at least 5 atm, not more than 5 atm, 5.5 atm, up to 5.5 atm, at least 5.5 atm, not more than 5.5 atm, 6 atm, up to 6 atm, at least 6 atm, not more than 6 atm, 7 atm, up to 7 atm, at least 7 atm, not more than 7 atm, 8 atm, up to 8 atm, at least 8 atm, not more than 8 atm, 9 atm, up to 9 atm, at least 9 atm, not more than 9 atm, 10 atm, up to 10 atm, at least 10 atm, not more than 10 atm, 11 atm, up to 11 atm, at least 11 atm, not more than 11 atm, 12 atm, up to 12 atm, at least 12 atm, not more than 12 atm, between 4 atm and 6 atm, or any range of two of the foregoing values.

The lobes 120 can be fully expanded against the surface of the vessel 10″. The vessel can expand from 10′ in FIG. 58D to 10″ in FIG. 58E. The one or more linear dissected lines can be formed in the vessel 10″. The one or more linear dissected lines can allow further expansion of the vessel from 10′ to 10″. In some methods, the vessel 10′ can be expanded 0.25 mm, 0.50 mm, 0.75 mm, 1 mm, or any range of two of the foregoing values to reach the vessel 10″. In some methods, the vessel 10′ can be expanded 10%, 20%, 30%, 40%, 50%, or any range of two of the foregoing values to reach the vessel 10″. The vessel 10′ can be slightly expanded due to the higher pressure of the fluid, wherein the pressure can be greater than 4 atm in some embodiments.

The pressure of the fluid can exert a force on the area 140 under the base of the strips 200. The pressure aids in maintaining the perpendicular orientation of the strips 200 with the raised features 300. The surface tension of the lobes 120 can aid in maintaining the perpendicular orientation of the strips 200 with the raised features 300. The pressure allows the strips 200 with the raised features 300 to further expand outward from the from the inner member 150 of the balloon 100. This further expansion of the strips 200 with the raised features 300 can facilitate crack propagation. The expansion of the balloon 100 and the crack propagation can be synergistic. As the balloon 100 expands, the linear dissected line lengthens by connecting more serrations, indentations, and/or microperforations. As the serrations, indentations, and/or microperforations connect to form a linear dissected line, the balloon 100 can further expand. The linear dissected line can be formed through the intimal tissue and into the medial plane in some methods.

The linear dissected line can slowly creep through the intimal tissue. The linear dissected line can slowly creep through into the medial plane. In some methods, the creation of the linear dissected line can take a period of time. This period of time can be 20 seconds, at least 20 seconds, greater than 20 seconds, 30 seconds, at least 30 seconds, greater than 30 seconds, 40 seconds, at least 40 seconds, greater than 40 seconds, 50 seconds, at least 50 seconds, greater than 50 seconds, 60 seconds, at least 60 seconds, greater than 60 seconds, or any range of two of the foregoing values. The period of time can be sufficient to allow the expanded strips 200 with the raised features 300 to seat within the tissue layers. The period of time can be sufficient to allow the linear dissected line to extend to medial layer. The period of time can be sufficient to allow the serrated lines to seat and initiate the serration effect as described herein.

The balloon 100 can facilitate vascular remodeling as described herein. The vessel 10′ can be expanded to the vessel 10″. In FIG. 58B, the vessel 10 can be contracted due to the diseased state. For instance, extensive stenosis can have constricted the vessel 10 thereby preventing flow. Even with the serrations, indentations, and/or microperforations formed, the vessel 10′ can still be constricted. The vessel 10″ can be expanded due to the serrated line formed by the joining of adjacent serrations, indentations, and/or microperforations under crack propagation. The serrated line can be predictably formed by the balloon 100. The balloon 100 can repeatedly form the same pattern of serrations, indentations, and/or microperforations based on the shape of the strips 200 with the raised features 300. The balloon 100 can repeatedly form the same pattern of one or more serrated line based on the shape of the strips 200 with the raised features 300. The balloon 100 can repeatedly form longitudinal serrated lines.

The strips 200 with the raised features 300 only disturb a small portion of the circumference of the vessel 10″. Compared to the circumference of the vessel 10″, the strips 200 with the raised features 300 have a small width. The width of the strip 200 with the raised features 300 can be a percentage of the of the circumference of the vessel 10″, such as 1%, 2%, 3%, 4%, 5%, 6%, 7%, 8%, 9%, 10%, 15%, less than 5%, less than 10%, or any range of two of the foregoing values.

The width of the lobe 120 can be a large percentage of the of the initial circumference of the vessel 10, such as 110%, 111%, 112%, 113%, 114%, 115%, 116%, 117%, 118%, 119%, 120%, less than 120%, less than 115%, more than 110% or any range of two of the foregoing values. The width of the lobe 120 can be a large percentage of the of the expanded circumference of the vessel 10″, such as 99%, 98%, 97%, 95%, 94%, 93%, 92%, 91%, 90%, 85%, more than 95%, more than 90%, less than 100%, or any range of two of the foregoing values. With the strips 200 with the raised features 300 being pushed into the vessel 10″, a series of deep and long crack can be formed. The crack can extend to the medial layer. The crack can be relatively thin corresponding generally to the width of the strip 200 with the raised features 300. The crack can be relatively long corresponding generally to the length of the strip 200 with the raised features 300. The crack can be relatively deep, extending deeper than the height of the strip 200 with the raised features 300. The crack can extend beyond the radially outwardly facing surface 304. The serrations, indentations, and/or microperforations can be formed and slowly combined into the linear dissected line over a time period of about 60 seconds, 65 seconds, 70 seconds, 75 seconds, 80 seconds, 85 seconds, 90 seconds, 95 seconds, 100 seconds, 115 seconds, 110 seconds, 115 seconds, 120 seconds, 125 seconds, 130 seconds, 135 seconds, 140 seconds, 145 seconds, 150 seconds, 155 seconds, 160 seconds, 165 seconds, 170 seconds, 175 seconds, 180 seconds, more than 60 seconds, more than 120 seconds, less than 180 seconds, or any range of two of the foregoing values.

The serrations, indentations, and/or microperforations can pull away from the strip 200 with the raised features 300 over time and under increased pressure by the balloon 100, thus forming a linear dissected line. The linear dissected line can allow for the low pressure expansion of the vessel 10″. After forming the one or more linear dissected lines, the balloon 100 can be deflated. The balloon 100 can be removed from the vessel. The vessel 10″ can remain expanded. The linear dissected line can allow for positive vessel remodeling. The linear dissected line can allow for substantial lumen gain. The linear dissected line can allow for reduction in recoil. As described herein, the vessel can experience far less recoil when forming one or more linear dissected lines. As described herein, the vessel can be remodeled back to the pre-diseased state, thereby increasing flow.

In some methods, one or more steps can be reversed. The balloon 100 inflation and deflation can be bi-directional. The balloon 100 can go from the inflate state to the deflated state. The balloon 100 can go from the deflated state to the inflated state. The strip 200 with the raised features 300 rotation from perpendicular to transverse can be bi-directional. The strip 200 with the raised features 300 can go from the perpendicular orientation to the transverse orientation. The strip 200 with the raised features 300 can go from the transverse orientation to the perpendicular orientation. By inflating and deflating the balloon 100, the strip 200 with the raised features 300 can rotate in both direction.

FIG. 58E illustrates the final resting balloon 100 with the fully expanded diameter. The strips 200 with the raised features 300 can be maintained in their perpendicular orientation. The balloon 100 can produce serrated lines through the intimal tissue and into the medial plane. The physician can hold the balloon 100 for a period of time, including 5 seconds, 10 seconds, 20 seconds, 30 seconds, 40 seconds, 50 seconds, 60 seconds, 120 seconds or any range of two or more of the foregoing values to allow the serrated lines to seat and initiate the serration effect. The balloon 100 can push the diseased vessel outward from vessel 10′ to vessel 10″. The serrations can slowly combine into the linear dissected line and pull away from the raised features 300. The balloon 100 can gently expand the diameter of the vessel 10″.

In some methods, the balloon 100 can be inflated typically at atmospheres of 4 or more. The pressure inside the lobes 120 can reach a maximum pressure. The maximum pressure can be 4 atm, 4.5 atm, 5 atm, 5.5 atm, 6 atm, 7 atm, 8 atm, 9 atm, 10 atm, 11 atm, 12 atm, between 4 atm and 6 atm, between 4 atm and 8 atm, or any range of two or more of the foregoing values. The lobes 120 can be expanded against the surface of the vessel 10″, thereby urging the vessel 10″ outward. The vessel 10″ can be expanded. The dissected lines can facilitate lumen gain and vascular remodeling, as described herein. The expansion of the balloon 100 and the crack propagation can function together to allow for vessel expansion.

In some embodiments, the radially outward facing surface 304 can be configured to contact a wall of the vessel 10 while creating little to no separation of plaque from the wall of the vessel 10. In some embodiments, the lobes 120 can be configured to exert a tensile force on the wall of the vessel 10 near the regions where the raised features 300 contact the vessel 10. In some embodiments, the lobes 120 can be configured to exert a force on the vessel 10 causing the vessel 10 to pull away from the raised features 300. In some embodiments, the lobes 120 can be configured to exert a force on the vessel 10 that allows the radially outward facing surface 304 to create serrations in the vessel 10. In some embodiments, the lobes 120 can be configured to exert a force on the vessel 10 that allows the radially outward facing surface 304 to create linear dissected lines. In some embodiments, the sloped side walls in combination with the expansion of the lobes 120 can be configured to produce a plurality of longitudinally oriented lines into or through one or more of the layers of the tissue. The radially outward facing surface 304 of each raised feature 300 can be configured for penetration through layers of the vascular anatomy. In some embodiments, the sloped side walls in combination with the expansion of the lobes 120 can be configured to produce a plurality of longitudinally oriented lines through the intimal layer. In some embodiments, the sloped side walls in combination with the expansion of the lobes 120 can be configured to produce a plurality of longitudinally oriented lines through the internal elastic lamina layer. In some embodiments, the sloped side walls in combination with the expansion of the lobes 120 can be configured to produce a plurality of longitudinally oriented lines into the medial layer. In some embodiments, the sloped side walls in combination with the expansion of the lobes 120 can be configured to provide lumen gain independent of the arterial dimension. In some embodiments, the sloped side walls in combination with the expansion of the lobes 120 can be configured to produce a plurality of longitudinally oriented lines to the medial layer, enabling greater arterial expansion that increases volumetric blood flow. In some embodiments, the sloped side walls in combination with the expansion of the lobes 120 can be configured to produce a plurality of longitudinally oriented lines to the medial layer that improves stenosis. In some embodiments, the sloped side walls in combination with the expansion of the lobes 120 can be configured to cause positive vessel remodeling. In some embodiments, the sloped side walls in combination with the expansion of the lobes 120 can be configured to maintain the generally perpendicular orientation of the raised features 300 as the raised features 300 induce nodes of separation in the intima. In some embodiments, the sloped side walls in combination with the expansion of the lobes 120 can be configured to change the pressure distribution at a wall of the vessel 10 allowing the raised features 300 to further penetrate the vessel 10. In some embodiments, the balloon 100 can be configured to deliver energy. In some embodiments, the strips 200 increase trackability and pushability by translating forces longitudinally along the balloon 100.

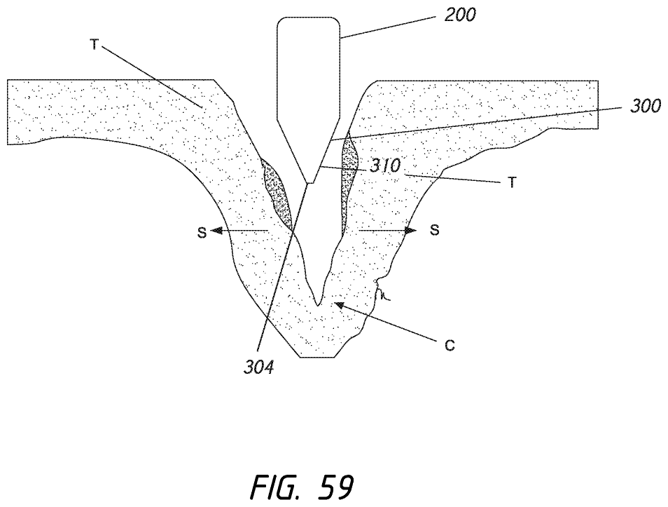

FIG. 59 illustrates that once the initial penetration of the raised feature 300 into the tissue T, the cleaving stress can overcome the need for penetration of the radially outwardly facing surface 304. The further expansion of the tissue through the advancing of the crack C into deeper tissue T can provide a tissue wake for the raised feature 300. In some methods, as the raised feature 300 penetrates the tissue T, the radially outwardly facing surface 304 does not contact the tissue T and the crack generation C can be aided by the side walls 310, 312 of the raised feature 300, the side walls 204 of the strip 200, and/or the strain S produced by the expansion of the balloon 100. Due to the design and nature of the strain S induced by the expansion of the balloon 100 and the radially outwardly facing surface 304, the advancing cleavage plane can quickly migrate into spaces beyond the radially outwardly facing surface 304 such that the radially outwardly facing surface 304 no longer is in direct contact with the leading edge of the crack. The cleavage plane can extend beyond the radially outwardly facing surface 304 of the raised feature 300.

During one or more of the above events described herein, the pressure wave can be generated. During one or more of the above events, the balloon 100 can be pulsated by the pressure wave. The balloon 100 can be inflated during the fully expanded period. The balloon 100 can be inflated to the base pressure in the balloon 100 during the fully expanded period. The balloon 100 can be pulsated with the pressure wave during the fully expanded period in some methods. The balloon 100 can be fully expanded. The vessel 10 can be fully expanded to vessel 10″. The balloon 100 can be pulsated by the pressure wave to hammer against the vessel 10. The balloon 100 can be pulsated by the pressure wave to expand the diameter of the vessel 10 to vessel 10″. The balloon 100 can be pulsated by the pressure wave to expand the diameter of the vessel 10′ to vessel 10″. The raised features 300 of the strips 200 can extend into the vessel 10. The raised features 300 of the strips 200 can be perpendicular to the surface of the balloon 100. The radially outward facing surfaces 304 of the raised features 300 of the strips 200 penetrate into the vessel 10 beyond the wall of the vessel during the fully expanded period. The balloon 100 can be pulsated with the pressure wave while the raised features 300 of the strips 200 penetrate into the vessel 10 in some methods. The raised features 300 of the strips 200 can penetrate into the vessel 10 without the balloon 100 being pulsated in some methods. The raised features 300 of the strips 200 can penetrate into the vessel 10 generating nucleation sites for the fissuring event. The relatively narrow profile of the raised features 300 of the strips 200 can penetrate into the vessel 10 The raised features 300 can penetrate the intercellular matrix in the vessel 10. The raised features 300 can minimize cellular injury by limiting microscopic tearing. The balloon 100 can be pulsated by the pressure wave while the raised features 300 of the strips 200 penetrate into the vessel 10 in some methods. The raised features 300 of the strips 200 can penetrate into the vessel 10 without the balloon 100 being pulsated by the pressure wave in some methods. The balloon 100 can be pulsated by the pressure wave after the raised features 300 of the strips 200 penetrate into the vessel 10 in some methods.

The balloon 100 can be pulsated by the pressure wave to facilitate the fissure in some methods. The balloon 100 can be pulsated by the pressure wave to provide lithotripsy in addition to the fissure. In some methods, the fissures quickly produce cracking along the intra-luminal surface of the vessel 10″. In some methods, due to the proximity and alignment of the cracks, the cracks join to become a long crack along the intra-luminal surface of the vessel 10″ in some methods. The cracks can extend along the entire length of the strip 200. The cracks can extend along less of the length of the strip 200 in some methods. The cracks can extend along greater than the length of the strip 200 in some methods. The depth of the penetration of the crack can be typically similar to the depth of medial tissue in some methods. The balloon 100 can be pulsated by the pressure wave to produce a lithotripsy effect. The balloon 100 can be pulsated to provide a synergistic response with serration caused by the raised features 300. The balloon 100 can be pulsated by the pressure wave to create vibrations at the surface of the lobes 120 of the balloon 100. The lobes 120 can be pulsated by the pressure wave to facilitate increasing the diameter of the vessel 10. The balloon 100 can be pulsated by the pressure wave to create vibrations for dynamical loading of the wall of the vessel. The balloon 100 can be pulsated by the pressure wave to create forces on the wall of the vessel to accelerate crack propagation. The balloon 100 can be pulsated by the pressure wave to create vibrations of the strips 200 with raised features 300. The strip 200 can be pulsated by the pressure wave to facilitate crack propagation. The raised features 300 can be pulsated by the pressure wave to facilitate crack propagation. The balloon 100 can be pulsated to produce any advantage described herein.

During any period during the method described herein, the system 1100 can utilize a base pressure. The base pressure can be provided by the inflation device 400 through the reservoir 854 in some methods. In some embodiments, the balloon 100 can be inflated to a low pressure between 2 atm and 4 atm in some methods. The balloon can be inflated to a base pressure. The balloon can be inflated to about 2 atm of pressure in some methods. The balloon can return to the base pressure after the pressure wave. During any period during the method described herein, the system 1100 can generate a pressure wave. In some methods, once the balloon 100 is at least partially inflated, the system 1100 can produce pressure waves. In some methods, once the balloon 100 is inflated to contact the wall of the vessel 10, the system 1100 can produce pressure waves. In some methods, once the balloon 100 is inflated to cause penetration of the raised features 300, the system 1100 can produce pressure waves. In some methods, once the balloon 100 is fully inflated, the system 1100 can produce pressure waves. The system 1100 can produce pressure waves during any period described herein. The pressure wave can be generated when the balloon 100 is inflated to 1 ATM, 1.5 ATM, 2 ATM, 2.5 ATM, 3 ATM, 3.5 ATM, 4 ATM, 4.5 ATM, 5 ATM, 5.5 ATM, 6 ATM, 7 ATM, 8 ATM, 9 ATM, 10 ATM, or any range of two of the foregoing values. The pressure wave can pass through the balloon 100 and the raised features 300. The pressure waves can create a shear stress on the calcium sheet. The pressure wave can accelerate the application of tensile force by the lobes 120. The pressure wave can accelerate penetration of the raised features 300 through the internal elastic lamina and into the calcium sheet. The raised features 300 can penetrate faster and/or deeper with pressure waves. The pressure wave can accelerate the expansion of the vessel 10 to the vessel 10″.

The pressure waves can be generated with the generator 800 of the system 1100. The motor 830 can be activated to translate the cam hammer 848, which strikes the piston 878 to deflect the membrane 884. The system 1100 can be activated to produce pressure waves. The pressure inside the balloon 100 can spike. The pressure inside the balloon 100 can periodically rise and fall with the striking of the membrane 884. The pressure inside the balloon 100 can periodically rise with pressure wave and fall to base pressure. The pressure inside the balloon can periodically rise and fall from about 2 atm to about 8 atm, from about 2 atm to about 10 atm, from about 2 atm to about 12 atm, from about 2 atm to about 14 atm, from about 2 atm to about 16 atm, from about 2 atm to about 18 atm, from about 2 atm to about 20 atm, or any range of two of the foregoing values. The pressure inside the balloon 100 can spike to press the raised features 300 into the plaque. The pressure inside the balloon 100 can spike to create microperforations. The pressure inside the balloon 100 can spike to cause wave propagation. The pressure inside the balloon 100 can rise and fall almost instantaneously. In some methods, the pressure inside the balloon can be maintained below a threshold. The peak pressure of the pressure wave can be below a threshold throughout the procedure.

The pressure inside the balloon 100 can rise and fall with each rotation of the motor shaft 831. The pressure inside the balloon 100 can rise and fall with each rotation of the cam can 860 and the cam can insert 862. The pressure inside the balloon 100 can rise and fall with each translation and retraction of the cam hammer 848. The pressure inside the balloon 100 can rise and fall with each strike of the piston 878. The pressure inside the balloon 100 can rise and fall with each deflection of the membrane 884. The pressure inside the balloon 100 can rise and fall based on the speed of the motor 830.

In some methods, all of the raised features 300 of the strips 200 can make contact with vessel wall simultaneously. In some methods, all of the raised features 300 of the strips 200 can rise and fall with the changing pressure of the balloon 100. In some methods, all of the raised features 300 along the length of the balloon 100 can move simultaneously. In some methods, all of the raised features 300 along the length of the balloon 100 can move radially outward with the rise in pressure with the balloon 100. In some methods, all of the raised features 300 along the length of the balloon 100 can move radially inward with the fall in pressure with the balloon 100.

In some methods, the raised features 300 of the strips 200 can follow the wave that propagates through the balloon 100. In some methods, the proximal raised features 300 of the strips 200 can move radially outward before the middle raised features 300 of the strips 200 move radially outward. In some methods, the middle raised features 300 of the strips 200 can move radially outward before the distal raised features 300 of the strips 200 move radially outward. In some methods, the raised features 300 of the strips 200 can undulate. In some methods, the raised features 300 of the strips 200 can move with a smooth up and down motion. In some methods, the raised features 300 of the strips 200 can undulate with the pressure wave. The raised features 300 can undulate with the pressure wave. The pressure wave can include a fluid wave that compresses fluid particles. The raised features 300 can undulate with the fluid wave. In some methods, the raised features 300 of the strips 200 can undulate at a microscopic level. In some methods, the raised features 300 of the strips 200 can undulate at the speed of sound. In some methods, the raised features 300 of the strips 200 can undulate along the length of the balloon 100 as the pressure wave propagates along the length of the balloon 100. In some methods, the strip 200 can undulate into plaque due, in part, to the non-circumferential connection between the strips 200. The proximal raised features 300 of one strip 200 can move independently of other proximal raised features 300 of another strip 200. The middle raised features 300 of one strip 200 can move independently of other middle raised features 300 of another strip 200. The distal raised features 300 of one strip 200 can move independently of other distal raised features 300 of another strip 200. The strips 200 can move independently of other strips 200. The strips 200 are not constrained circumferentially. The strips 200 can be unconnected to other strips 200. The strips 200 can be unconnected along the circumference of the balloon 100. The strips 200 can undulate due to the unconnected nature of the strips 200. The strips 200 can undulate due to the elongate nature of the strips 200. The strips 200 can undulate due to the design of the strip 200. The strip 200 can be slender strips of material. The column strength of the strips 200 can facilitate the balloon being pushed along the vessel. The strip 200 can be configured to limit axial compression. The column strength of the strip 200 can limit sideways deflection. The strip 200 can be configured to limit bending in certain directions. The strip 200 can be designed to bend in the direction of undulation. The strip 200 can be designed to remain rigid in other directions. The strip 200 can have structural asymmetry. The design of the strip 200 can allow for controlled flexibility. For example, the features of FIGS. 25A-25K can facilitate bending in certain directions. The strip 200 can bend for undulation by adding notches or reliefs at regular intervals. The reliefs can have as hinges that only operate in one direction. The strips 200 can undulate due to bending direction of the strips 200.

As described herein, the raised features can move outwardly and inwardly with the pressure wave. The raised features 300 can move outwardly with the rise in pressure. In some methods, the raised features 300 can zipper into the plaque. The raised features 300 can produce corresponding spaces in the plaque. This space can be the nucleation site or serration. The raised features 300 can fit into corresponding spaces in the plaque. The raised features 300 can interlock with the plaque. The raised features 300 can interlock with the vessel wall. The raised features 300 can form interlocking projections with the vessel. As described herein, the raised features 300 can move inwardly with the fall of pressure to base pressure. In some methods, the raised features 300 can zipper out of the plaque. The raised features 300 can pull away from corresponding spaces in the plaque. The raised features 300 can disengage with the plaque. The raised features 300 can disengage with the vessel wall.

The raised features 300 can zipper under the influence of the pressure wave. The raised features 300 can engage and disengage with the vessel by the change in pressure from the pressure wave. The raised features 300 can engage and disengage with the vessel by a force applied along the length of the strip 200. The raised features 300 can engage with the vessel at the peak positive pressure of the pressure wave. The raised features 300 can disengage with the vessel when the pressure returns to base pressure. The raised features can zipper into and out of the corresponding spaces, at least partially. The raised features 300 can remain within the corresponding spaces when advanced. The raised features 300 can remain within the corresponding spaces when retracted. The motion of the raised features 300 can be micromotion. The motion of the raised features 300 can be less than the height of the raised features 300. The motion of the raised features 300 can be less than the height of the nucleation site. In some methods, the raised features 300 can form a zipper like feature. The raised features 300 can be teeth along a flexible material. The hardened material with corresponding spaces can be teeth. The raised features 300 can interlock with the corresponding spaces. The raised features 300 can separate from the corresponding spaces.

The zippering into the plaque under the influence of the pressure wave can create unexpected results. The inward and outward motion can create spaces for cavitation. The pressure wave helps the raised features to zipper by increasing the pressure within the balloon. The increase in pressure can cause radially expansion of the balloon 100. The radial expansion of the balloon 100 can cause the zippering of the raised features. The spaces in the plaque can be formed by the hammering motion of the raised features 300. The raised features 300 can impact the plaque to form nucleation sites. The nucleation sites are small indentations into the surface of the plaque. The zippering creates corresponding spaces between the raised features when the raised features are retracted. These spaces allow cavitation bubbles to be formed and imploded. The cavitation can occur in these spaces. The cavitation can occur in the liquid within the vessel. The cavitation can occur when local pressure drops below the vapor pressure of the liquid. The cavitation can cause microjets within the spaces. The cavitation can cause additional pressure waves, such as shock waves, to fracture calcium. Additional features of cavitation are described herein.

The zippering effect can be a repeated motion of the raised features 300 driven by the pressure wave. The zippering effect can be micromotion of the raised features 300 into and out of the corresponding spaces or nucleation sites. The zippering can occur after the raised features 300 produce the corresponding space in plaque. The zippering can occur after penetration of the raised feature 300. The raised feature 300 is designed to penetrate to create the serration or microperforation. The zippering can be a distinct movement caused by the pressure wave. The zippering can be a movement into and out the serration. The zippering can cause the raised feature to move into the serration and move out of the serration. The zipper in can corresponding to movement to occupy more of the serration by the raised feature. The zipper out can correspond to movement to occupy less of the serration by the raised feature 300. In some embodiments, the zippering can appear to be interlocking of the raised feature 300 with the hardened surface. The zippering can occur after the penetration described herein. The raised feature 300 can be designed to penetrate the vessel to create serrations. The pressure of the system can be raised until the raised feature 300 penetrates to create serrations. The zippering can occur after penetration. The zippering can occur when the spaces are created in the plaque. The zippering can be a separate effect driven by the pressure wave.

While not to be limited by a theory, the zippering of the raised features 300 can cause the cavitation. The raised features 300 can be solid object moved through the liquid of the vessel. The movement of the raised features 300, specifically the high-speed movement of the raised features 300 caused by the pressure wave, can cause cavitation. The high-speed movement of the raised features 300 can reduce the local static pressure below the vapor pressure of the fluid, causing the fluid to create vapor bubbles. The high-speed movement of the raised features 300 can create high velocity areas causing pressure to drop and bubbles to form. These cavitation bubbles can collapse creating pressure waves. The high-speed movement of the raised features 300 can create pressure waves outside of the body of the balloon 100. The high-speed movement of the raised features 300 can create pressure waves within the serrations. These pressure waves can travel through the tissue and fracture calcium. The pressure waves created by cavitation bubbles can be in ultrasonic range. The pressure waves created by the cavitation bubbles can be between 20 kHz and 10 MHz. The pressure waves created by the cavitation bubbles can have the same frequency as the pressure waves created by the generator 800. The pressure waves created by the cavitation bubbles can have a different frequency than the pressure waves created by the generator 800.

While not to be limited by a theory, the hammering motion of the raised features 300 can cause cavitation. The movement of the raised features 300 can cause cavitation in addition to the impact of the raised features 300 with the hammering motion. The high-speed movement of the raised features 300 can create vapor bubbles, which can collapse creating pressure waves. The cavitation can create pressure waves in addition to the impact forces created by the hammering motion. The cavitation can amplify the fracturing forces delivered to the calcium.

The strip 200 and the raised features 300 can undulate under the influence of the pressure wave. The undulation can be a rolling motion of the strip 200 with the raised features 300. The undulation can concentrate the pressure wave to a single raised feature 300. In some methods, the raised features 300 of the strips 200 can have a smooth rising and falling form along the length of the strip 200. In some methods, the raised features 300 of the strips 200 can have a rolling motion. In some methods, the raised features 300 of the strips 200 do not move simultaneously. In some methods, the raised features 300 of the strips 200 move in a wave. In some methods, a portion of raised features 300 of the strips 200 can move into engagement with the plaque while a portion of raised features 300 of the strips 200 are not in engagement with the plaque. In some methods, the strip 200 can zipper into plaque due, in part, to the non-circumferential connection between the strips 200. The proximal raised features 300 of one strip 200 can move independently of other proximal raised features 300 of another strip. The proximal raised features 300 of one strip 200 can move independently of other proximal raised features 300 of another strip 200. In cases when the wavefront propagates in a plane, along the length of the balloon 100 from the proximal end to the distal end, more than one strip 200 can move together in synergy with each other. When the wavefront is planar the proximal raised features 300 of one strip 200 can move at the same time of other proximal raised features 300 of another strip 200. The middle raised features 300 of one strip 200 can move independently of other middle raised features 300 of another strip 200. The distal raised features 300 of one strip 200 can move independently of other distal raised features 300 of another strip 200. The strips 200 can move independently of other strips 200. The strips 200 are not constrained circumferentially. The strips 200 can be unconnected to other strips 200. The strips 200 can be unconnected along the circumference of the balloon 100. The strips 200 can zipper due to the unconnected nature of the strips 200. The strips 200 can zipper due to the elongate nature of the strips 200. The strips 200 can zipper due to the column strength of the strips 200. The strips 200 can zipper due to bending direction of the strips 200.

The crack can propagate under the influence of the pressure wave. As described herein, the lobes 120 of the balloon 100 can impart a strain on the wall of the vessel. This strain can cause the hardened material to tear along the spaces between the serrations. This strain can cause the hardened material to tear in less predictable directions but follow lines of low energy calcium bonds. The pressure wave can influence the strain produced by the balloon 100. The lobes 120 can be in contact with the majority of the vessel wall. The lobes can be applying a strain when the pressure wave is generated. The pressure wave can increase the strain applied by the balloon 100. The pressure wave can produce dynamic loading on the vessel wall. The pressure wave can change the strain over time. The pressure wave can cause the balloon 100 to vibrate. The pressure wave can induce vibrations. The pressure wave can induce stress fluctuations. The pressures wave can change the strain periodically. The pressure waves can change the strain at the pulse frequency of the pressure waves. The pressures wave can impart a dynamic load which exerts greater force than static loads. This dynamic loading by the pressure wave can shorten the time of crack propagation. This dynamic loading by the pressure wave can shorten the time of crack formation. This dynamic loading by the pressure wave can shorten the time to lumen gain. This dynamic loading by the pressure wave can shorten the time to segmentation of the hardened material. This dynamic loading by the pressure wave can cause controlled cracking along the serrations. This dynamic loading by the pressure wave can cause longitudinal formation of cracks following the longitudinal orientation of the serrations. This dynamic loading by the pressure wave can cause rapid cracking along regions of lower energy calcium bonds.

The strain effect caused by the pressure wave can be different than the zippering effects, hammering effects, and undulation effects described herein, in some embodiments. The zippering effects, hammering effects, and undulation effects can relate to the strip 200 and the raised features under the influence of the pressure waves. The strain effect can relate to the lobes 120 of the balloon 100 under the influence. Separate and distinct from the zippering effects of the strips 200 with raised features 300 penetrating the tissue driven by the pressure wave energy is the strain effect of the tissue due to the lateral forces on the vessel wall due to the pressure wave propagation through the balloon body. The balloon 100 in its inflated state can be modeled as a cylinder with strips 200 with raised features 300 on the outer surface of the balloon 100. Adding to the balloon cylinder, there can be a pressure wave that enters the proximal end of cylinder, traveling at or near the speed of sound produced by the generator 800. The front of the pressure wave is displacing a defined amount of volume, determined by the displacement of the membrane 884 at the generator. The volume change produces a localized diameter shift in the cylindrical balloon body of the balloon 100. The diameter shift adds tension at the walls of the balloon body of the balloon 100 during the diameter growth. As observed in experiments of cadaveric diseased lesions, the tension at the walls of the balloon body of the balloon 100 produced by the pressure wave can add a separate and immediate localized tension to the vessel wall along the exact region where the strips 200 with the raised features 300 are entering the tissue. This additional strain produced by the pressure wave can drive an accelerated, but controlled cracking of the hardened material or plaque. The strain effect can produce controlled separation. The strain effect can produce controlled dissection.

In some methods, the crack propagation can start at any point along the balloon. The pressure wave can induce a strain along the ballon length. The pressure wave can induce a strain along the ballon circumference. The strain from the pressure wave can be applied along the entire circumference of the balloon. In some methods, the crack propagation can follow the direction of the pressure wave. In some methods, the crack propagation can begin at the proximal serrations and propagate toward distal serrations. In some methods, the crack propagation can follow the undulation of the strip 200. The crack can propagate as the serrations are formed. In some methods, the crack propagation can start at the proximal end of the strip 200 with raised features 300 that has penetrated the tissue. The cracking can occur as quick as the pressure wave movement through the balloon. The cracking can occur along one, two, or three lines of penetrated tissue. The cracking can occur along the intima, media or both. The cracking can break up calcium sheets. The cracking can break up circumferential calcium into one, two, or three arcs of calcium.

The lobes 120 of the balloon 100 can deliver pressure waves. The lobes 120 can be in contact with the majority of the vessel wall. The lobes 120 can deliver acoustic waves to tissue when the pressure wave is generated. The lobes 120 can deliver acoustic waves with a combination of frequencies, including ultrasonic frequencies. The lobes 120 of the balloon 100 can directly contact the vessel wall to deliver energy. When cracking occurs, the arcs of calcium can be more fragile and less stable. At the same time as the cracking, or after the cracking occurs, these fragile arcs of calcium are more susceptible to the pressure wave breaking the calcium into small calcium pieces. The lobes 120 can deliver pressure waves to the tissue before cracking. The lobes 120 can deliver pressure waves to the tissue at the same time as cracking. The lobes 120 can deliver pressure waves to the tissue after cracking. The cracking can thin out the intima layer limiting interference to the pressure wave reaching the calcified disease. The cracking can allow pressure wave to more effectively reach the calcified disease. The cracking can increase luminal diameter. The cracking can be minimally damaging to the vessel. The cracking can reduce flow limiting dissections. The lobes 120 can be involved with both cracking by producing dynamic strain by the pressure wave and fracturing calcium by delivering the acoustic wave to tissue. The dynamic strain can correlate to the increase in pressure in the balloon 100 caused by the pressure wave. In some embodiments, the lobes 120 contribute significantly to calcium fracturing. The lobes 120 can be in contact with the majority of the vessel. The pressure waves delivered around the circumference of the balloon 100 can pass through the lobes 120. The pressure waves can pass into the tissue causing a shearing stress on calcium. The lobes 120 can directly contact the vessel wall to more effectively deliver energy to the tissue.

While not to be bound by a theory, the strips 200 can allow limited movement in certain directions. In some methods, the strip 200 can allow bending in certain directions. In some methods, the strip 200 have a size and shape to allow the rolling motion of the strip 200 under the influence of the pressure wave. In some methods, the dimensions of the strip 200 can allow the rolling motion. In some methods, the material of the strip 200 can allow the rolling motion. In some methods, the physical properties of the strip 200 can allow the rolling motion. In some methods, the material properties of the strip 200 can allow the rolling motion. The pressure wave can apply a load along the length of the strip 200 as the wave propagates distally. The pressure wave can introduce bending to the strip 200. The bending of the strip 200 can accelerate the formation of microperforations. The bending of the strip 200 can accelerate the formation of cracks.

In some methods, the balloon 100 can apply a force on the vessel under the influence of the pressure wave. The pressure wave can increase the pressure of the balloon 100. The balloon 100 can apply a tensile force on the vessel 10. The balloon 100 can pull on the vessel wall under the increased pressure. The balloon 100 can cause the vessel 10 to stretch. The balloon 100 can cause the vessel 10 to expand radially outward. The pressure wave can facilitate vessel expansion. The pressure wave can facilitate vessel expansion before penetration of the raised features 300. The pressure wave can facilitate vessel expansion after penetration of the raised features 300. The pressure wave can accelerate crack formation expansion after penetration of the raised features 300. The pressure wave can increase the pressure of the balloon 100. The balloon 100 can apply a tensile force on the vessel 10. The tensile force can cause the vessel to pull apart from the perforations thereby forming cracks. The cracks can propagate when tensile stress is applied perpendicular to the crack plane. The balloon 100 can apply tensile stress by the pressure wave that drives propagation.

The system 1100 can generate the pressure wave. The system 1100 can generate the acoustic waves that travels from the balloon 100. The system 1100 can be used to disrupt calcium bonds within the vessel 10. The system 1100 can be used to disrupt calcium bonds of superficial plaque. The system 1100 can be used to disrupt calcium bonds of plaque adhered to the internal elastic lamina. The system 1100 can be used to disrupt calcium bonds beyond the internal elastic lamina. The system 1100 can be used to disrupt calcium bonds at the intimal layer. The system 1100 can be used to disrupt calcium bonds at the medial layer. The system 1100 can be used to selectively disrupt calcium bonds. The system 1100 can be used to disrupt calcium bonds along the length of the balloon 100. The system 1100 can be used to disrupt calcium bonds beyond the distal end of the balloon 100. The system 1100 can be used to disrupt calcium bonds beyond the proximal end of the balloon 100. The system 1100 can be used to disrupt calcium bonds along the entire length of the balloon 100. The system 1100 can be used to disrupt calcium bonds greater than the length of the balloon 100. The system 1100 can modify calcified regions deep within the vessel 10. The system 1100 can modify highly calcified regions.

The system 1100 can be used to generate pressure waves in the balloon 100. The pressure waves can allow the treatment of plaque by concurrently creating microperforations and disrupting calcium bonds. The pressure waves can allow the treatment of plaque by disrupting of calcium bonds in plaque after creating microperforations in plaque. The pressure waves can allow the treatment of plaque by creating microperforations in plaque after disrupting of calcium bonds in plaque. The system 1100 can create pressure waves before forming microperforations. The system 1100 can create pressure waves at the same time as forming microperforations. The system 1100 can create pressure waves after forming microperforations. The system 1100 can create pressure waves before forming serrated lines. The system 1100 can create pressure waves at the same time as creating serrated lines. The system 1100 can create pressure waves after forming serrated lines. The system 1100 can create pressure waves between forming serrated lines and forming cracks. The system 1100 can create pressure waves between forming serrated lines and forming cracks to accelerate crack formation. The system 1100 can create pressure waves before forming longitudinal cracks. The system 1100 can create pressure waves at the same time as creating longitudinal cracks. The system 1100 can create pressure waves after forming longitudinal cracks. In some methods, the pressure wave can be a low pressure spike for instance under 12 atm. The pressure wave can increase the pressure to be between 2 atm and 10 atm within the balloon 100. The pressure wave can increase the pressure within the balloon 100 from the base pressure. The pressure wave can be greater within the tissue. The raised features 300 can amplify the pressure wave within the tissue. The raised features 300 can amplify the pressure wave 1000× to 40,000× the pressure wave within the balloon 100. The pressure wave can increase the pressure below a threshold value. The pressure within the balloon 100 can be maintained below a threshold. The pressure wave can be significantly below the burst pressure of the balloon 100. The peak pressure of the pressure wave can be 2× less, at least 2× less, 5× less, at least 5× less, 10× less, at least 10× less, 20× less, at least 20× less, than the burst pressure of the balloon 100, or any range of two of the foregoing values. The balloon 100 can withstand the pressure wave without bursting. The balloon 100 can withstand the pressure wave without failing. The raised features 300 can reduce the need for high pressure. The system 1100 can create a pressure wave at low pressure ranges of the balloon 100. The pressure wave can facilitate breaking up the calcified regions. The pressure wave can alter disease morphologies. The pressure wave can smooth and compress plaque. The pressure wave can facilitate microperforation creation. The pressure wave can facilitate crack propagation. The pressure wave can break up calcium bonds in calcium deposits within the vessel. The pressure wave can be within a desired frequency range. The pressure wave can be within a desired pressure range within the tissue. The raised features 300 concentrate the energy delivery to close to a point source. The raised features 300 can reduce the surface area for energy delivery. The raised features 300 can be small in total surface area compared to the surface area of the balloon 100. The raised features 300 can amplify the delivery of energy due to the reduced surface area.

The methods can treat any anatomy described herein. The methods can treat the entire lower extremity. The methods can function as direct therapy for a treatment site. The methods can function as adjustive vessel preparation. The methods can be used for below-the-knee treatment. The methods can be used for below-the-ankle treatment. The methods can be used for above-the-knee treatment. The methods can be used for AV fistula access. The methods can be used for treatment from the iliac artery to the digital vessels of the foot.

### Deflation Period

The inflation device 400 can remove fluid from the reservoir 854 which can deflate the balloon 100. The balloon 100 can begin to deflate. The balloon 100 can deflate from the maximum pressure. The pressure can gradually decrease over a period of time, including 5 seconds, 10 seconds, 20 seconds, 30 seconds, 40 seconds, 50 seconds, 60 seconds, or any range of two or more of the foregoing values. The decreased pressure can be 4 atm or less. The balloon 100 can deflate in a similar manner as shown in FIG. 58D. The vessel 10″ can remain at the expanded diameter during deflation. The vessel 10″ can permanently retain the expanded diameter. In some methods, the vessel 10″ may experience very slight recoil, which can be significantly less than traditionally angioplasty. The recoil effect can be dampened due to the permeant linear dissected lines created by the balloon 100 and the strips 200 with the raised features 300. The linear dissected lines can create segments of the vessel 10″. The segments can be separated by the linear dissected lines to the medial layers. These segments can be less likely to recoil into the smaller diameter of the vessel 10′ or the vessel 10.

The decreased pressure within the balloon 100 exerts a lesser force at the base of the strips 200 during deflation. Even at lower pressures, the strips 200 can maintain their perpendicular orientation. The pressure can decrease to between 2 atm and 4 atm in some methods. The raised features 300 on the strips 200 can retract from the permeant linear dissected lines. The raised features 300 on the strips 200 can retract radially inward. The retraction can occur faster than the expansion. The expansion can be slowed to allow the serrated lines to seat and initiate the serration effect. The retraction can be quicker since the permanent linear dissected lines are created. The raised features 300 on the strips 200 can easily retract from the expanded vessel 10″

The balloon 100 can continues to deflate under decreasing pressure. The pressure inside the lobes 120 can fall. The lobes 120 can decrease to about 2 atm. The pressure can decrease gradually from 4 atm to 2 atm, such as deceasing to 2 atm, 2.5 atm, 3 atm, 3.5 atm, 4 atm, or any range of two or more of the foregoing values. The lobes 120 can deflate from their position against the surface of the vessel 10″. In some methods, the lobes 120 and the portion 140 under the strips 200 can deflate about equally. In some methods, the lobes 120 can deflate slightly before the portion 140 under the strips 200. In some methods, the portion 140 under the strips 200 can deflate slightly before the lobes 120. The strips 200 with the raised features 300 can maintain their perpendicular orientation during a portion of the deflation. The lobes 120 can be positioned on either side of the strip 200 with the raised features 300. The lobes 120 can exert a pressure to maintain the perpendicular orientation of the strip 200 200 with the raised features 300.

The strip 200 with the raised features 300 can gradually move inward as the balloon 100 deflates. The strip 200 with the raised features 300 can contract radially relative to the inner member 150 of the balloon 100. The strips 200 with the raised features 300 can retract out from the permeant linear dissected lines. The strips 200 with the raised features 300 can retract out from the internal elastic lamina. The strips 200 with the raised features 300 can be disposed between the lobes 120 during retraction. The strips 200 with the raised features 300 can maintain their orientation relative to the lobes 120 during retraction. The lobes 120 can guide the strips 200 with the raised features 300 during retraction. The strip 200 with the raised features 300 can move radially inward. The strip 200 with the raised features 300 can move radially inward toward the inner member 150 of the balloon 100. The portion 140 of the balloon 100 under the base of the strip 200 can continue to deflate as the pressure decreases. The vessel 10″ can maintain the expanded state while the balloon 100 deflates. The expansion of the vessel 10″ can be due to the permeant linear dissected lines. The permeant linear dissected lines can reconstruct the vessel 10″ to increase flow. In some methods, the balloon 100 may not be needed to support the vessel 10″ in the expanded state after the permeant linear dissected lines are formed. Further, the permanent linear dissected lines can reduce the need for lumen support such as stents.

The strip 200 with the raised features 300 can retract into the space between the lobes 120. The balloon 100 can deflate in a similar manner as shown in FIG. 58C. The vessel 10″ can remain at the expanded diameter during deflation. The vessel 10″ can permanently retain the expanded diameter. In some methods, the vessel 10″ does not recoil as the balloon 100 deflates. The strip 200 with the raised features 300 can maintain a perpendicular orientation during deflation. The portion 140 of the balloon 100 under the base of the strip 200 can be small relative to the lobes 120. The geometry of the area 140 of the balloon 100 under the base of the strip 200 can facilitate retraction. The narrow strips 200 with the raised features 300 can easily fit between the lobes 120 during deflation. The strips 200 with the raised features 300 can displace fluid toward the lobes 120 during deflation. The strips 200 with the raised features 300 can retract inward faster than the lobes 120 can deflate. The strip 200 with the raised features 300 can be configured to move radially inward at the low pressure. In some methods, the pressure can be 4 atm or less when the strips 200 with the raised features 300 fall below the circumference of the lobes 120. The fluid can fill less than a majority of the balloon 100. The lobes 120 can begin to pull away from the vessel 10″. The lobes 120 can be deflated such that only a small portion of the circumference of the balloon 100 contacts the vessel 10″. The pressure can continue to fall. The pressure can be below 2 atm. The strips 200 with the raised features 300 can remain in the perpendicular orientation at low pressure.

The pressure in the balloon 100 can continue to fall, such as less than 0.5 atm, less than 1 atm, less than 2 atm. The low pressure in the balloon 100 can facilitate the folding the strips 200 with the raised features 300 to the desired orientation. The decreasing pressure can rotate the strips 200 with the raised features 300 from the perpendicular orientation. The pressure in the lobes 120 continues to decrease. The lobes 120 can act on the strips 200 with the raised features 300 during deflation. The deflation of the lobes 120 can cause rotation of the strips 200 with the raised features 300 from the perpendicular orientation.

The portion of the lobe 130 against the sides of the strip 200 with the raised features 300 can deflate. The portion 130 can apply a force or torque on the strip 200 with the raised features 300 to orient the strip 200 with the raised features 300 transversely during deflation. Two adjacent lobes 120 can apply a force to the strip 200 with the raised features 300. The two adjacent lobes 120 can apply a pressure to orient the strip 200 with the raised features 300 transversely. During this phase of the deflation, the pressure of the fluid can be under 2 atm in some methods. The loss of pressure can be sufficient to rotate the strip 200 with the raised features 300. The strip 200 with the raised features 300 can rotate from the perpendicular orientation shown in FIG. 58C to the transverse orientation in FIG. 58B. The balloon 100 can include one or more intermediate orientations as the strip 200 with the raised features 300 rotates. This slope of the side walls of the strip 200 with the raised features 300, in combination with the deflation of the balloon 100, can allow for effective control of the orientation of the strips 200. With the lobes 120 deflating in the regions near the strip 200, the strip 200 with the raised features 300 can be effectively positioned into the optimized transverse orientation. The vessel 10″ can remain expanded during this further deflation of the balloon 100. The balloon 100 and the strips 200 with the raised features 300 can be out of contact with the vessel 10″. The strips 200 with the raised features 300 can fold due in part on the dynamics of deflation of the balloon 100 including the shape of the strips 200 with the raised features 300 and the lobes 120. The strip 200 with the raised features 300 can assume a flattened, rotated state when the balloon 100 is deflated. The strip 200 with the raised features 300 can undergo rotation as the balloon 100 deflates. The strip 200 with the raised features 300 can be considered self-orienting. The strip 200 with the raised features 300 can rotate to the proper orientation during deflation. The lobes 120 can provide repeatable and predicable orientation of the strips 200 with the raised features 300 during deflation

The strip 200 with the raised features 300 can rotate to the transverse orientation as shown in FIG. 58A. The strip 200 with the raised features 300 can return to a tangential orientation. The strip 200 with the raised features 300 can be positioned under the wing 110 of the balloon 100. As the lobes 120 deflate, the lobes 120 can partially cover the strips 200 with the raised features 300. The balloon 100 can deflate within the expanded vessel 10″. This series of illustrations in reverse can show subsequent deflation. The strips 200 with the raised features 300 can rotate during deflation of the balloon 100. The strips 200 with the raised features 300 can return to their delivery or tangential orientation. The strips 200 with the raised features 300 can be at least partially or fully covered by the balloon 100 after deflation. The strips 200 with the raised features 300 can lie down after deflation. The deflation of the balloon 100 can cause the strips 200 with the raised features 300 to turn from the perpendicular orientation to the tangential orientation. The strips 200 with the raised features 300 can rotate within the expanded vessel 10″.

The balloon 100 can be folded to a low profile. The balloon 100 can include one or more pleats. The number of pleats can correspond to the number of strips 200. The pleats can be configured to at least partially cover the strip 200 with the raised features 300 when the balloon 100 is deflated. The strip 200 with the raised features 300 can be disposed between the pleat and a surface of the balloon 100 when the balloon 100 is deflated. The pleat can form the wing 110. The wing 110 can be a portion of the balloon 100 that overlies a portion of the strip 200 with the raised features 300 when the balloon 100 is deflated. The strips 200 with the raised features 300 can be at least partially covered by the balloon 100 once the balloon is deflated. The strips 200 with the raised features 300 can be at least partially covered by the balloon 100 for removal from the vessel. The strips 200 with the raised features 300 can be at least partially covered by the balloon 100 for movement to another location in the vessel. The balloon 100, in particular the wing 110, can serve as a buffer between the vessel 10 and the raised features 300 of the strips 200 during any movement within the vessel 10 or retrieval. The balloon 100 can cover the radially outwardly facing surface 304 of the raised features 300 of the strip 200. The radially outwardly facing surface 304 of the raised features 300 of the strip 200 can be covered to protect the vessel 10 during movement or retrieval. The strip 200 with the raised features 300 can be in a tangential orientation during movement or retrieval. The tangential orientation allows the strips 200 with the raised features 300 to have a low profile retrieval configuration.

The balloon 100 can be placed at another location within the same vessel 10. The balloon 100 can be moved in the deflated state. The balloon 100 can be subsequently inflated to create permanent linear dissected lines. The balloon 100 can be redeployed within the same vessel 10 without retrieval. The balloon 100 can be moved longitudinally within the same vessel 10. The balloon 100 can be rotated within the same vessel 10. The balloon 100 can create permanent linear dissected lines at two or more locations along the vessel 10. The balloon 100 can be subsequently deflated and inflated along two or more locations.

In some methods, the permanent linear dissected lines from at a first location and the permanent linear dissected lines from a second location can merge. For instance, the balloon 100 can be shifted longitudinally within the vessel 10 about equal to the length of the strips 200. The balloon 100 can be moved longitudinally but not rotated. The permanent linear dissected lines from the first location can align with permanent linear dissected lines of the second location. The balloon 100 can create permanent linear dissected lines longer than the length of the strip 200 by subsequent deployments. The balloon 100 can be subsequently deflated and inflated to create permanent linear dissected lines of the desired length.

The balloon 100 can be placed at another location within the another vessel. The balloon 100 can be moved to an adjacent vessel. The balloon 100 can be moved to a paired vessel. The balloon 100 can be moved in the deflated state. The balloon 100 can be subsequently inflated to create permanent linear dissected lines. The balloon 100 can reconstruct two or more vessels before retrieval. The balloon 100 can create permanent linear dissected lines at two or more locations.

The balloon 100 can be retrieved from the patient's vasculature. The balloon 100 can be moved through vasculature. The balloon 100 can be moved in the deflated state. The balloon 100 can have a low profile configuration similar to the delivery configuration. The strips 200 with the raised features 300 can be at least partially covered during retrieval.

Inch Worming within a Vessel

The diseased state may have severely constricted the vessel such that the balloon 100 cannot reach the treatment site in some methods. The vessel may be such a small diameter than the balloon 100 cannot pass. The vessel can include total occlusion. The vessel can include hardened material that constricts the vessel. The deposits can completely occlude the vessel.

The balloon 100 can be expanded proximally to the treatment site. The system 1100 can produce pressure waves that travel beyond the balloon 100. The system 1100 can produce pressure waves that travel beyond the distal end of balloon 100. The system 1100 can produce pressure waves that propagate distally. The pressure waves can provide a lithotripsy effect distal to the balloon 100. The pressure waves can reduce calcification. The pressure waves can deliver energy to the treatment site. The pressure waves can pass through soft tissue. The pressure waves can create microfractures. The pressure waves can break calcium into small fragments. The pressure waves can shatter calcium bonds. The calcium can remain adhered to the vessel wall. The calcium can remain within the medial layer. The calcium can remain within the intima layer. The pressure waves can cause structural stress to the calcium matrix. The pressure waves can cause curvilinear deposits to break into smaller arcs. The pressure waves can cause ring-like deposits to segment. The pressure waves can cause deposits to break apart. The pressure waves can treat hardened occlusions. The pressure waves can treat severe calcifications. The pressure waves can treat superficial calcium at the treatment site. The pressure waves can treat deep calcium deposits. The pressure waves can fracture calcium distal to the balloon 100 to expand the vessel diameter. The pressure waves can fracture calcium to allow the balloon 100 to reach the treatment site. The pressure waves can increase flexibility of the vessel distal to the balloon 100. The pressure waves can allow for vessel expansion. The pressure waves can make the vessel more compliant to allow the balloon 100 to reach the treatment site. The pressure waves can allow for easier expansion by blood flow.

The vessel can expand by the pressure waves only, in some methods. The vessel can expand while the balloon 100 is proximal to the treatment site. The vessel can expand without the balloon 100 pressing against the treatment site. Instead, in some methods, the pressure wave can travel distally for calcium fracturing. By fracturing calcium, the vessel becomes more compliant allowing for expansion of the vessel by blood flow. The vessel can be expanded to allow the balloon 100 to access the treatment site. The system 1100 can be used in angulated lesions. The system 1100 can be used in tortuous lesions. The system 1100 can be used in uncrossable lesions by applying pressure waves to the treatment site to expand the vessel. The treatment site can be treated with pressure waves before the balloon 100 is positioned at the treatment site. The system 1100 can be used for severe calcifications. The system 1100 can be used in area where the balloon 100 cannot cross the lesion and reach the treatment site. The system 1100 can debulk calcium with the pressure waves before moving the balloon 100 to the treatment site.

The balloon 100 can be deflated. The balloon 100 can be deflated when moved within the vessel. The balloon 100 can be moved to the treatment site. The balloon 100 can reach the treatment site due to the vessel expansion by the pressure waves. The balloon 100 can reach the treatment site due to pressure waves creating a lithotripsy effect distal to the balloon 100. The balloon 100 can expand at the treatment site. The balloon 100 can expand to increase the diameter of the vessel at the treatment site. The subsequent use of the balloon 100 at the treatment can fracture additional calcium. The subsequent use of the balloon 100 at the treatment site can lead to additional lumen gain.

The systems can perform any method described herein at the treatment site. The balloon 100 can be inflated to the base pressure at the treatment site. The balloon 100 can be pulsated with the pressure waves at the treatment site. The raised features 300 can create serrations at the treatment site. The generator 800 can deliver pressure waves to the treatment site.

While the balloon 100 is at the treatment site, the pressure waves can travel distal to the distal end of the balloon 100. The balloon 100 can treat the vessel distal to the balloon 100 while at the treatment site. The pressure waves can propagate distally from the balloon 100. The pressure waves can propagate to fracture calcium distal to the treatment site. The pressure waves can provide lithotripsy beyond the length of the balloon 100. The pressure waves can provide lithotripsy beyond the distal end of the balloon 100. The pressure waves can make the vessel more compliant along a length greater than the length of the balloon 100. The pressure waves can allow for easier expansion downstream of the treatment site. The pressure waves can allow for better blood flow downstream of the treatment site.

The balloon 100 can be deflated. The balloon 100 can be deflated when moved within the vessel. The balloon 100 can be moved downstream of the treatment site. The systems can perform any method described herein downstream of the treatment site. The balloon 100 can be moved to another location within the vasculature. The systems can perform any method described herein at another location.

The balloon 100 can designed based on the occlusion profile at the treatment site. The balloon 100 can be a non-compliant balloon. The balloon 100 can be a cylindrical shaped balloon. The balloon 100 can expand to the diameter of the occlusion. The balloon 100 can be a rigid device that maintains a fixed diameter. The balloon 100 can expand to the diameter of the corresponding vessel. The balloon 100 can be rigid to expand highly calcified vessels. The balloon 100 can expand to a nominal size. The balloon 100 can be configured to minimize dog-boning. The balloon 100 can treat chronic occlusions where higher pressures may be needed. The balloon 100 can open the stenotic area through the use of serrations and/or pressure waves, as described herein. The balloon 100 can maintain a constant diameter to acuate the carrier 1200. The balloon 100 can be used to treat the underlying cause of the obstruction. The balloon 100 can be used for calcified lesion dilation. The balloon 100 can be used for pre-dilation for complex lesions.

The balloon 100 can be compliant. The balloon 100 can dog-bone relative to occlusion. The balloon 100 can expand beyond a nominal size. The balloon 100 can have a non-fixed diameter. The balloon 100 can conform to the vessel shape. The balloon 100 can be expanded with lower pressures. The balloon 100 can be flexible. The balloon 100 can be highly-stretchable. The balloon 100 can confirm to irregular vessel shapes. The balloon 100 can conform to superficial plaque. The balloon 100 can conform to irregular cross-sections of the vessel. The balloon 100 can dog-bone. The end portions of the balloon 100 can over-expand while the center portion near the occlusion stays constrained. The balloon 100 can expand to the vessel wall. The balloon 100 can be constrained along the length of the balloon 100 by the lesion. The occlusion can create uneven pressure. The balloon 100 can stretch on either side of the occlusion. The balloon 100 can have uneven expansion.

The pressure waves can provide a lithotripsy effect relative to the occlusion. The pressure waves can travel radially outward from the balloon. The pressure waves can travel longitudinally. The pressure waves can travel longitudinally into the occlusion. The pressure waves can travel transversely into the occlusion. The ends of the balloon 100 can direct the pressure wave into the occlusion. The ends of the balloon 100 can be expanded to a greater diameter, thereby creating a ring or curvilinear segment near the occlusion. The pressure waves can be generated from this ring or curvilinear segment and travel into the occlusion. During treatment, the waist of the balloon 100 can be reduced as the occlusion lessens. The balloon 100 can expand to nearly or the full diameter of the vessel.

The balloon 100 can treat various disease morphology. The balloon 100 can be non-compliant to treat highly diseased vessel, which may require higher pressures to expand the vessel. The balloon 100 can be compliant to conform to the vessel. The balloon 100 can reshape the vessel through expansion of the balloon 100, creation of serrations, and/or the delivery of the pressure waves. The balloon 100 can be semi-compliant, combining the features described herein.

### Cavitation

The balloon 100 with the strips 200 and the raised features 300 can be used to create cavitation bubbles outside of the balloon 100. The cavitation bubbles can be vapor-filled spaces. The cavitation bubbles can be created by the negative pressure generated by the pressure wave. The cavitation bubbles can be created by ultrasound generated by the pressure wave. The cavitation bubbles can be created by blunt impact of the raised features 300 against the surface of the vessel and/or the plaque. The cavitation bubbles can collapse to create shock waves. In some embodiments, the cavitation bubbles are entirely outside the balloon 100 with the strips 200 and the raised features 300. In some embodiments, the cavitation bubbles can be formed within the microperforations formed by the raised features 300. The microperforations can act as voids or wells for the cavitation bubbles. The cavitation bubbles can be used therapeutically for lithotripsy.

The raised features 300 can contribute to the formation of cavitation bubbles. In some embodiments, the combination of the imperfections in the raised features 300 that rise above the balloon surface of the balloon 100 and the ultrasonic frequency of the pressure wave moving through the raised features 300 can produce localized cavitation bubble formation. In some embodiments, the surface roughness or small imperfections in the material of the raised features 300 can act as nucleation sites for cavitation bubbles. These imperfections of the raised features 300 can lower the energy barrier required for cavitation bubble formation, allowing cavitation to initiate. The regional wedged zone or area of the microperforations, that was generated by the penetration and retraction of the raised features 300 throughout the delivery of the pressure wave, can produce a localized pressure well for cavitation to initiate. The localized cavitation can more easily occur under low-frequency ultrasound in combination with the reginal acoustic pressure of the pressure wave. The ultrasound frequencies of the pressure wave can be between 20,000 Hz and 40,000 Hz in some embodiments. Additional frequencies below 20,000 Hz are often present in the pressure wave in some embodiments. Additional frequencies above 40,000 Hz are often present in the pressure wave in some embodiments. The ultrasound of the pressure wave can propagate as alternating compression and rarefaction waves, causing local reductions in pressure during the rarefaction cycles. When the negative pressure exceeds the threshold for cavitation in the body, cavitation can occur.

The cavitation bubble can collapse near the surface of the raised feature 300, thereby producing a high-speed microjet or shockwave. The effect of this cavitation bubble implosion can impact the calcium surface producing a surface effect which can weaken the calcium and facilitate fracture lines. The effect of this cavitation bubble implosion can impact deeper calcium within the vessel wall to weaken the calcium. When the cavitation bubble growth is rapid and the cavitation bubble collapse is violent, it can be termed inertial cavitation. This inertial cavitation can occur when the radius of the bubble grows rapidly under low pressure, then the surrounding fluid pushes inward as pressure rises. The implosion of the bubble can generate shock waves/or microjets, which can erode nearby solid surfaces. The implosion of the bubble can generate shock waves and/or microjets, which can fracture nearby calcified surfaces. The implosion of the bubble can generate shock waves and/or microjets, which can fracture calcium bonds of embedded calcium. The implosion of the bubble can generate shear forces that impact calcium bonds of surrounding tissue. The implosion of the bubble can fracture calcium for lithotripsy.

In some embodiments, the cavitation bubble can generate a pressure wave that may have different characteristics from the pressure wave generated by the generator 800. The acute or shock like event of the cavitation can be used to generate a pressure wave with a frequency in the acoustic spectrum. In some embodiments, an acute or shock like event of the cavitation can produce a pressure wave with a frequency in the ultrasound spectrum. In some embodiments, an acute or shock like event of the cavitation can produce a pressure wave with a frequency in both the acoustic and the ultrasound spectrums. In some embodiments, generating the change in pressure can produce a wave through cavitation of a bubble. In some embodiments, generating the change in pressure can comprise transitioning between two discrete levels of pressure over time at a constant frequency. In some embodiments, generating the change in pressure can comprise transitioning between more than two discrete levels of pressure over time at a constant frequency. In some embodiments, generating the change in pressure can comprise transitioning between two discrete levels of pressure over time at a variable frequency. In some embodiments, generating the change in pressure can comprise transitioning between more than two discrete levels of pressure over time at a variable frequency. In some embodiments, generating the change in pressure can reduce the time for lesion yielding. In some embodiments, generating the change in pressure can minimize neighboring tissue damage.

In some embodiments, the propensity of the cavitation and cavitation implosion can be random. Experimentally it has been observed that once cavitation occurs in a region, the cavitation is self-promoting thereby facilitating more cavitation events to occur. In addition, when cavitation occurs rapidly in a narrow space, like that generated when a single raised element 300 penetrates the intima and produces a narrow well or microperforation, the well or microperforation can act like a pump compressing and expanding the cavitation bubbles. This compression and expansion can promote cavitation implosion due to the rapid and repeated pressure changes occurring in a narrow zone of the microperforation.

### Carrier Methods

FIG. 61 shows the carrier 1200. The carrier 1200 can be positioned at a treatment site. The carrier 1200 can be an additional structure coupled to the balloon 100. The carrier 1200 can be used in any method described herein. The balloon 100 can be collapsed for delivery within a vessel. The carrier 1200 can be collapsed for delivery within the vessel. The carrier 1200 can have a low-profile configuration. In some embodiments, the balloon 100 and the carrier 1200 can be collapsed within a sheath for delivery. The carrier 1200 can overlap with the balloon 100. The carrier 1200 can overlap with the body of the balloon 100. The raised features 300 can be folded against the carrier 1200 for delivery. The raised features 300 can be in a tangential orientation for delivery. The carrier 1200 can provide column strength to the balloon 100 during delivery. The carrier 1200 can facilitate delivery to the treatment site. In some methods, the vessel can be pre-dilated. The vessel can be dilated with the balloon 100. The vessel can be dilated with another balloon. The vessel can be pre-dilated before delivery of the carrier 1200.

The carrier 1200 can be expanded at the treatment site. The carrier 1200 can be self-expanding. The carrier 1200 can expand upon deployment from the sheath. The carrier 1200 can expand when the user holds the carrier 1200 while pulling back on the sheath to expose the carrier 1200. The carrier 1200 can expand when the user holds the inner member 150 of the balloon 100 while pulling back on the sheath to expose the carrier 1200. The carrier 1200 can self-expand during a controlled delivery technique. The carrier 1200 can self-expand when removed from a constraint. In some embodiments, the carrier 1200 can expand to position the raised features 300 inward from the vessel wall. In some embodiments, the carrier 1200 can expand to position the raised features 300 against the vessel wall. In some embodiments, the carrier 1200 can expand while the raised features 300 do not penetrate. The carrier 1200 can self-expand from a delivery state to a partially expanded state. The carrier 1200 can be expanded by the balloon 100 to the fully expanded state. The carrier 1200 can self-expand from a delivery state to the fully expanded state.

The balloon 100 can expand the carrier 1200. The balloon 100 can be inflated to expand the carrier 1200. The balloon 100 can engage with the carrier 1200 to expand the carrier 1200. The carrier 1200 can be expanded by radial forces of the balloon 100. The carrier 1200 can be expanded by longitudinal forces of the balloon 100. The carrier 1200 can expand from the partially expanded state to the fully expanded state by the balloon 100 in some methods. The balloon 100 can be inflated to fully expand the carrier 1200. The balloon 100 can be expanded by the inflation device 400 to expand the carrier 1200. The carrier 1200 can be expanded within the vessel. The carrier 1200 can be delivered to the treatment site within the vessel. The treatment site can be a location of vessel narrowing caused by plaque. The carrier 1200 can configured to expand under the inflation of the balloon 100. In some embodiments, the carrier 1200 can self-expand and further expand by inflation of the balloon 100. The balloon 100 can expand the carrier 1200 against the wall of the vessel. The balloon 100 can cause the raised features 300 to penetrate. The balloon 100 can be inflated to the base pressure. The balloon 100 can be inflated in a controlled manner. The balloon 100 can be inflated to expand the vessel. The balloon 100 can be expanded to acuate the carrier 1200. The balloon 100 can be expanded to acuate the raised features 300.

The generator 800 can generate a pressure wave during any period of the method. The balloon 100 can be pulsated by the pressure wave. The balloon 100 can be inflated to expand the carrier 1200. The balloon 100 can be inflated to the base pressure in the balloon 100 to expand the carrier 1200. The balloon 100 can be pulsated with the pressure wave to expand the carrier 1200 in some methods. The balloon 100 can be fully expanded. The balloon 100 can be fully expanded against the vessel. The balloon 100 can be pulsated by the pressure wave to hammer against the vessel. The balloon 100 can be fully expanded against the carrier 1200. The balloon 100 can be pulsated by the pressure wave to hammer against the carrier 1200. The balloon 100 can be pulsated by the pressure wave to expand the diameter of the vessel. The generator 800 can be a source of energy for the carrier 1200.

The raised features 300 of the carrier 1200 can have a hammering motion to create serrations. The raised features 300 of the carrier 1200 can have a zippering motion to create cavitations. The raised features 300 of the carrier 1200 can extend into the vessel. The raised features 300 of the carrier 1200 can be perpendicular to the surface of the balloon 100. The raised features 300 of the carrier 1200 can be perpendicular to the surface of the carrier 1200. The radially outward facing surfaces 304 of the raised features 300 of the carrier 1200 can penetrate the vessel beyond the wall of the vessel. The balloon 100 can be pulsated with the pressure wave while the raised features 300 of the carrier 1200 penetrate the vessel in some methods. The raised features 300 of the carrier 1200 can penetrate the vessel without the balloon 100 being pulsated in some methods. The raised features 300 of the carrier 1200 can penetrate the vessel generating nucleation sites for crack formation. The relatively narrow profile of the raised features 300 of the carrier 1200 can penetrate the vessel. The raised features 300 of the carrier 1200 can penetrate the intercellular matrix in the vessel. The raised features 300 of the carrier 1200 can minimize cellular injury by limiting microscopic tearing. The balloon 100 can be pulsated by the pressure wave while the raised features 300 of the carrier 1200 penetrate the vessel in some methods. The raised features 300 of the carrier 1200 can penetrate the vessel without the balloon 100 being pulsated by the pressure wave in some methods. The balloon 100 can be pulsated by the pressure wave after the raised features 300 of the carrier 1200 penetrate the vessel in some methods. In some embodiments, the raised features 300 cannot undulate. The carrier 1200 can circumferentially connected such as a stent. The carrier 1200 can circumferentially connect the raised features which can limit or prevent undulation. In some embodiments, the carrier 1200 can be configured to allow the raised features to undulate.

The balloon 100 can be pulsated by the pressure wave to facilitate the crack propagation in some methods. In some methods, the cracking can be along the intra-luminal surface of the vessel. In some methods, due to the proximity and alignment of the perforations by the raised features 300, the cracks join to become a crack along the intra-luminal surface of the vessel in some methods. The cracks can extend along the entire length of the carrier 1200. The cracks can extend along less than the length of the carrier 1200 in some methods. The cracks can extend along greater than the length of the carrier 1200 in some methods. The depth of the penetration of the crack can be typically similar to the depth of medial tissue in some methods. The carrier 1200 can be pulsated by the pressure wave to facilitate crack propagation. The raised features 300 can be pulsated by the pressure wave to facilitate crack propagation The balloon 100 can be pulsated by the pressure wave to provide lithotripsy in addition to the crack propagation.

The balloon 100 can be pulsated by the pressure wave for lithotripsy. The balloon 100 can be pulsated to break calcium bonds. The balloon 100 can be pulsated to reduce a thickness of calcium. The balloon 100 can be pulsated to fracture calcium into smaller particles. The balloon 100 can be pulsated by the pressure wave to create vibrations at the surface of the carrier 1200. In some embodiments, the balloon 100 is separated from the vessel wall. In some embodiments, the balloon 100 is not in direct contact with the vessel. In some embodiments, the balloon 100 does not impart strain on the vessel wall. In some embodiments, the carrier 1200 can be configured to allow the balloon 100 to be in direct contact with the vessel wall.

The balloon 100 can be pulsated by the pressure wave to facilitate increasing the diameter of the vessel. The balloon 100 can be pulsated by the pressure wave to create vibrations for dynamical loading of the wall of the vessel. The balloon 100 can be pulsated by the pressure wave to create forces on the wall of the vessel to accelerate crack propagation. The balloon 100 can be pulsated by the pressure wave to create vibrations of the carrier 1200. The balloon 100 can be pulsated by the pressure wave to create vibrations of the raised features 300. The balloon 100 can be pulsated to produce any advantage described herein.

The balloon 100 can be inflated to a base pressure. The base pressure can expand the carrier 1200. The base pressure can press the carrier 1200 against the vessel wall. The base pressure can press the raised features 300 against the vessel wall. The base pressure can be provided by the inflation device 400 through the reservoir 854 in some methods. In some embodiments, the balloon 100 can be inflated to a low pressure between 2 atm and 4 atm in some methods. The balloon 100 can return to the base pressure after the pressure wave. In some methods, once the balloon 100 is at least partially inflated, the generator 800 can produce pressure waves. In some methods, once the carrier 1200 is expanded to contact the wall of the vessel, the generator 800 can produce pressure waves. In some methods, once the balloon 100 is inflated to cause penetration of the raised features 300, the generator 800 can produce pressure waves. In some methods, the generator 800 can produce pressure waves at any period during the method.

The pressure waves can be generated with the generator 800 as described herein. The pressure inside the balloon 100 can spike. The pressure inside the balloon 100 can periodically rise and fall with the striking of the membrane 884. The pressure inside the balloon 100 can periodically rise with the peak pressure of the pressure wave and fall to base pressure. The pressure inside the balloon can periodically rise and fall from about 2 atm to about 8 atm, from about 2 atm to about 10 atm, from about 2 atm to about 12 atm, from about 2 atm to about 14 atm, from about 2 atm to about 16 atm, from about 2 atm to about 18 atm, from about 2 atm to about 20 atm, or any range of two of the foregoing values. The pressure inside the balloon 100 can spike to press the raised features 300 of the carrier 1200 into the plaque. The pressure inside the balloon 100 can spike to create channels for drug delivery. The pressure inside the balloon 100 can spike to create microperforations. The pressure inside the balloon 100 can spike to cause crack propagation. The pressure inside the balloon 100 can spike to cause lithotripsy. The pressure inside the balloon 100 can spike to seat the carrier 1200 against the vessel. The pressure inside the balloon 100 can spike to seat the raised features 300 into the vessel. The pressure wave can accelerate positioning of the carrier 1200. The pressure wave can accelerate driving the raised features 300 into tissue and/or plaque. The pressure wave can accelerate anchoring of the carrier 1200. The pressure wave can accelerate the procedure. The pressure wave can modify lesion morphology. The pressure wave can change vessel compliance. The pressure wave can reduce recoil.

In some methods, all of the raised features 300 of the carrier 1200 can contact the vessel wall simultaneously. In some methods, all of the raised features 300 of the strips 200 can rise and fall with the changing pressure of the balloon 100. In some methods, all of the raised features 300 of the carrier 1200 can move simultaneously. In some methods, all of the raised features 300 of the carrier 1200 can move radially outward with the rise in pressure within the balloon 100. In some methods, all of the raised features 300 of the carrier 1200 can move radially inward with the fall in pressure within the balloon 100. In some methods, the raised features 300 of the carrier 1200 can move together. In some methods, the raised features 300 of the carrier 1200 do not move independently. In some methods, the raised features 300 of the carrier 1200 do not undulate with the pressure wave. In some methods, the raised features 300 of the carrier 1200 do not zipper with the pressure wave. In some embodiments, the carrier 1200 can be constrained circumferentially. In some embodiments, the carrier 1200 can be connected by interlocking struts. In some embodiments, the carrier 1200 can be connected along the circumference of the balloon 100.

The carrier 1200 can allow limited movement in certain directions. In some methods, the carrier 1200 can expand with the pressure wave. In some methods, the carrier 1200 can contract with the pressure wave. In some methods, the carrier 1200 can have limited radial motion.

The carrier 1200 can provide a supportive structure. The carrier 1200 can provide temporary support to the vessel. The carrier 1200 can remain expanded for a period of time, e.g., 1 second, 5 seconds, 10 seconds, 20 seconds, 30 seconds, 60 seconds, 90 seconds, 120 seconds, 3 minutes, 5 minutes, 30 minutes, or any range of two of the foregoing values. The balloon 100 can be deflated. The carrier 1200 can be recaptured. The carrier 1200 can be retrievable.

In some embodiments, the balloon 100 can expand the carrier 1200. The balloon 100 can engage with the carrier 1200 to expand the carrier 1200. The carrier 1200 and the balloon 100 can be removed from the vessel together. In some embodiments, the carrier 1200 can be completely removed from the vessel. The carrier 1200 can be retrievable. The carrier 1200 can be a temporary stent. The balloon 100 can be used to deliver the carrier 1200 to more than one treatment site. The balloon 100 can be used to deliver the carrier 1200 to multiple treatment sites in the same procedure, such as two, three, four, five, or any range of two of the foregoing values. The balloon 100 can collapse the carrier 1200 in some embodiments. The balloon 100 can engage with the carrier 1200 to collapse the carrier 1200. The balloon 100 and the carrier 1200 can be removed once the balloon 100 and the carrier 1200 are collapsed.

The balloon 100 can be removable from the carrier 1200, in some embodiments. The balloon 100 can be completely removed from the vessel, while the carrier 1200 remains. The carrier 1200 can provide permanent support to the vessel. The carrier 1200 can remain within the vessel. The carrier 1200 can be detached from the balloon 100. The raised features 300 can engage and anchor the carrier 1200 against the vessel wall. The carrier 1200 can function as a permanent stent. The carrier 1200 can maintain the expanded diameter of the vessel. The carrier 1200 can reinforce collapsed vessels. The raised features 300 can limit or prevent migration of the carrier 1200. In some embodiments, the balloon 100 can collapse while the carrier 1200 remains within the vessel. The balloon 100 can be removed from the vessel. The balloon 100 can be used to deliver another carrier 1200.

The systems described herein can increase luminal diameter by expanding the carrier 1200. The systems described herein can modify the lesion by penetrating with the raised features 300. The systems described herein can change the morphology of plaque. The systems described herein can break calcium bonds with the pressure wave of the generator 800. The systems described herein can change compliance of the vessel. The systems described herein can reduce recoil. The systems described herein can improve wound healing. The systems described herein can improve vessel patency. The systems described herein can provide results similar to a stent, while not leaving a structure behind. The systems described herein can reduce vessel injury and dissections. The systems described herein can reduce restenosis. The systems described herein can reduce neointimal hyperplasia. The systems described herein can enable vessel remodeling. The systems described herein can reduce procedure time.

### Advantages and Results

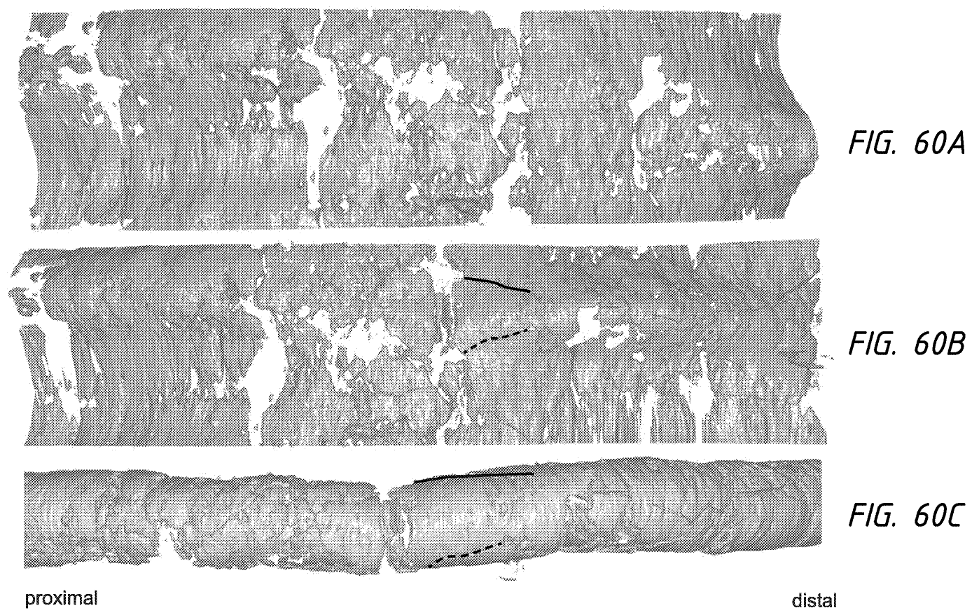

In some embodiments, disclosed herein are systems and methods that produce unexpected results clinically including improved linear fracturing with pressure wave generation and calcium fracturing through the lobes of the balloon, among other features. FIGS. 60A-60C show a set of views of the calcium sheets taken by microCT. FIG. 60A illustrates the flattened exterior view of the vessel before treatment with the system 1100 described herein. FIG. 60B illustrates the flattened exterior view of the vessel after treatment with the system 1100 described herein. This view shows a series of linear fractures that run longitudinally along the axis of the artery. As described herein, the linear fractures can occur faster with pressure wave generation. As described herein, the fracturing can be controlled to occur along longitudinal serrations. FIG. 60B illustrates two linear fractures that occurred in the vessel. The vessel also has additional calcium fracturing of the calcium sheets. Additionally, it can be seen that the fractures are not all aligned with the strip 200 with the raised features 300. This indicates that fracturing occurred in the lobes 120 of the balloon 100 between strips 200. The lobes 120 can deliver pressure waves directly to the calcium sheet that the lobes are in contact with. The pressure waves delivered by the lobes 120 can fracture calcium. The pressure waves delivered by the lobes 120 can reduce the thickness of calcium. The pressure waves delivered by the lobes 120 can shear the calcium sheets. FIG. 60C illustrates the normal 3D view of the vessel after treatment with the system 1100 described herein. In this experiment, only the right side of the artery was treated.

In some embodiments, disclosed herein are systems and methods that produce unexpected results clinically by increasing the number of fractures. FIGS. 66A-66B illustrate additional data from evaluations of the system and methods described herein using pressure wave generation with the generator 800 compared the balloon 100 with the strips 200 and the raised features 300 without pressure wave generation. The experiments were performed with parameters described herein. The study utilized four arteries, and eight lesions. Four of the lesions were treated with the balloon 100 with the strips 200 and the raised features 300 without pressure wave generation (labeled Serranator™). Four of the lesions were treated with the balloon 100 with the strips 200 and the raised features 300 with pressure wave generation of the generator 800 (labeled Sonic IVL). The systems treated both concentric calcifications (greater than 180 degrees) and eccentric calcifications (less than 180 degrees). FIG. 66A shows the comparison of the percentage of the fracture incidence. The percentage presence of fractures was 50% without pressure wave generation. The percentage presence of fractures was 75% with pressure wave generation. The percentage of fracture incidence significantly increased from 50% to 75% with pressure wave generation using the systems and methods described herein. The total number of fractures after pressure wave generation was greater than with systems without pressure wave generation. FIG. 66B illustrates the number of fractures in the frame. The number of fractures in the frame was about 0.5 without pressure wave generation and 1.0 with pressure wave generation. The fractures in the frame doubled with pressure wave generation. While the balloon 100 with the strip 200 and the raised features 300 created fractures well in areas of concentric calcification, using the same balloon 100 with pressure wave generation can further facilitate fractures in both eccentric and concentric calcifications. While not to be bound by a theory, the increase percentage of fracture incidence can be driven by the shear forces of the pressure waves when encountering calcium. The shear stress can be the primary mechanism in intravascular lithotripsy in some methods. The increase percentage of fracture incidence can be directly correlated with the pressure wave generated which delivers ultrasonic frequency to the vessel. The pressure wave can increase the percentage of fractures by creating stress that create fractures in the calcium. The pressure waves generated can create shear and tensile stress within the calcium deposits.

In some embodiments, disclosed herein are systems and methods that produce unexpected results clinically by orienting the fractures. FIG. 67 are summary images of fractures on the flattened outside view of the calcium. The views on the left were treated with the balloon 100 with the strips 200 and the raised features 300 without pressure wave generation (labeled Serranator™). The views on the right were treated with the balloon 100 with the strips 200 and the raised features 300 with pressure wave generation of the generator 800 (labeled Sonic IVL). As described herein, there can increase in the number of fractures with pressure wave generation. The views on the right shown an increase in the number of fractures with one cycle of pressure wave generation. There can be a measurable increase in the number of fractures with a single pressure wave cycle. The views on the right shown an increase in the number of fractures with three cycles of pressure wave generation. While not to be bound by a theory, a single pressure wave cycle may be sufficient. The fracturing of one cycle and three cycles appeared the same and/or substantially similar, in at least some experiments. The views on the right shown an increase in linearity with the fractures with pressure wave generation. While not to be bound by a theory, the increase in linearity can be driven by the raised features 300 that direct the pressure wave to the vessel. The raised features 300 can amply the pressure waves as described herein. The raised features can be longitudinally oriented and thereby create longitudinally oriented serrations. These serrations can serve a nucleation sites for both crack propagation and for additional lithotripsy effects. The pressure waves can cause fractures to propagate along weakened sections of the cellular matrix. The weakness can be caused by the serrations and/or the pressure waves, with both acting synergistically to weaken linearly oriented sections of the cellular matrix. The serrations can cause the weakened sections to facilitate lithotripsy effects of the pressure waves, and the pressure waves can weaken the cellular matrix to more easily form the serrations. These effects can occur simultaneously, in some methods, leading to both serration and pressure waves contributing to the linearity of fractures. The experiments shows fracturing in the calcium adjacent to the lobes 120. The lobes 120 can deliver pressure waves to calcium. The lobes 120 can deliver pressure waves with ultrasonic frequency for calcium fracturing. The lobes 120 can create hairline fracturing. The lobes can create fracturing along weakness in the cellular matrix. The lobes 120 can be in direct contact with the vessel while delivering pressure waves, thereby increasing the efficiency of energy delivery.

In some embodiments, disclosed herein are systems and methods that produce unexpected results clinically by reducing the time for crack propagation, thereby reducing the time of vessel occlusion and procedure time. In some embodiments, disclosed herein are systems and methods that produces linear cracks through serration preparation in tissue by raised features 300. In cardiovascular disease, applying interventional methods to increase lumen size in occluded lesions can aid in blood flow and can increase the likelihood that the vessel will remain patent longer than when minimal lumen gain can be achieved post-procedurally. Methods for increasing lumen diameter have a range of options for intravascular devices. On the basic end, Plain Old Balloon Angioplasty (POBA) or the use of percutaneous transluminal angioplasty (PTA) or similar approaches can be used to open the diseased lesion. In addition, more specialty devices such as the cutting balloon, AngioSculpt (Spectranetics), Chocolate (Cordis), and others that provide a mechanism to aid or control the balloon energy. Often products in this general category provide external structures on the surface of the balloon (either attached or not) that can be configured to contact the wall first and be pressed into the wall surface with the balloon pressure. The theory can be that the structures on the outer surface produce a localized increase in the force on the lumen which in turn can be intended to aid in allowing the surface to be incised and along with the balloon expansion enables arterial expansion. While these designs offer some advantages over POBA or plain balloons alone, they all have limitations on their effectiveness and their ability to facilitate lumen expansion especially in the complexity of diseases they might be used in. Further, the vessel can be occluded by the balloon during the time to press features into the wall surface. Temporary blood vessel occlusion should be limited to minimize ischemia risk. The systems and methods described herein can utilize pressure waves to shorten the time for occlusion of the blood vessel. The pressure wave can shorten the time to create serrations. The pressure wave can shorten the time to create linear cracks. The pressure wave can short the time for vascular remodeling. The time reduction can be 10×, for instance, shortening the time for crack propagation from 28.4 seconds to 2.7 seconds as shown in FIG. 56. FIGS. 54 and 55 show additional data related to the dramatic reduction of time. Reducing the time for crack propagation can reduce the time that the vessel is occluded by the balloon. Reducing the time for crack propagation can also reduce the total time for the procedure. This reduction in procedure time can minimize risks of infections, reduce the duration of anesthesia, and reduce complications which may necessitate longer hospital stays. This reduction in procedure time increase operating room capacity. This reduction in procedure time can reduce surgeon fatigue and reduce surgical errors. This reduction in procedure time can lower costs for the procedure.

In some embodiments, disclosed herein are systems and methods that produce unexpected results clinically by having a pulse frequency that allows for the system to reset to the base pressure after each pressure wave. The pulse frequency can be the number of pressure waves per second, measured in Hz. The systems and methods can utilize lower frequency for pressure wave generation, such as under 5 Hz. The systems and methods can have less than five pressure waves per second. The systems and methods can have less than five pressure impulses per second. The systems and methods can have less than 300 pressure waves per minute. The systems and methods can produce pressure waves that occur at a low frequency or repetition. The systems and methods can produce less than or equal to 180 pressure waves per minute. The systems and methods can produce less than or equal to 120 pressure waves per minute. The systems and methods can produce less than or equal to 60 pressure waves per minute. This rate of pressure wave generation can be superior for lithotripsy compared with systems that operate above 5 Hz. This rate of pressure wave generation can allow for better cavitation. This rate of pressure wave generation can lead to better calcium fragmentation. This rate of pressure wave generation can lead to higher success rates related to blood flow. This rate of pressure wave generation can lead to better clinical outcomes. This rate of pressure wave generation can reduce vascular damage. While not to be bound by a theory, the slower rate can allow more time for the calcium to fracture between pressure waves. Other higher-frequency or rapid-fire systems, such as those above 5 Hz, above 10 Hz, above 100 Hz, can increase inflammation. Other higher-frequency or rapid fire systems, such as those above 5 Hz, above 10 Hz, above 100 Hz, can cause injury to the vessel. Other higher-frequency or rapid fire systems, such as those above 5 Hz, above 10 Hz, above 100 Hz, can be painful to the patient. While not to be bound by a theory, the slower rate can allow the strip to undulate with the rise and fall of the pressure. The undulation can concentrate the energy to one or more raised features in contact with the hardened deposit. The undulation can allow some raised features to make contact with the deposit while other raised features do not make contact. While not to be bound by a theory, this undulation can impart more force than if all raised features were to rise at the same time. The undulation can shorten the time for serration formation. The undulation can shorten the time for crack formation.

In some embodiments, disclosed herein are systems and methods that produce unexpected results clinically by having a short pulse width for precision applications. The systems and methods can have an extremely short pulse width, such as a few microseconds. Other systems can have a pulse duration of the order of 10,000 to 100,000 microseconds. The pressure wave can last only about one to five microseconds. The pressure wave can be near-instantaneous. The systems and methods can have a pressure increase or rise time of about 10 to 100 nanoseconds. The systems and methods can have a positive phase or high pressure phase about one microsecond. The systems and methods can produce shock waves. The systems and methods can produce narrow-width pulses.

In some embodiments, disclosed herein are systems and methods that produce unexpected results clinically by having mechanical actuation of the membrane. The systems and methods can convert mechanical energy into pressure waves. The mechanical impact energy provided by the piston can be more consistent and accurate than pneumatic impact energy. The mechanical impact energy provided by the piston does not rely on air pressure from a compressor. The mechanical impact energy provide by the piston can be repeatable. The mechanical impact energy provide by the piston can be more precise for the generation of the pressure wave. The system and methods can allow for multiple frequency and amplitude settings. The system and methods can allow for more control over delicate tasks. The system and methods can provide superior precision. The system and methods can provide consistent, repeatable results. The system and methods can motor-driven mechanical motion. The system and methods can be ideal for consistent generation of the pressure wave.

In some embodiments, disclosed herein are systems and methods that produce unexpected results clinically related to lithotripsy. The systems and methods described herein are physical embodiments that have been created and successfully tested to perform the intended purpose of lithotripsy in the vascular of the human body. The systems and methods can work in both the human body as well as simulated conditions. The systems and methods can consistently deliver pressure waves with ultrasonic frequencies. The systems and methods can consistently deliver pressure waves with ultrasonic frequencies between 20 kHz and 125 kHz. The systems and methods can consistently deliver pressure waves with ultrasonic frequencies between 20 kHz and 200 kHz. The systems and methods can consistently fracture calcium utilizing pressure waves described herein. The systems and methods can treat severely calcified plaque in the vessels of the human body. The systems and methods can use localized pressure waves that travel along the length of the balloon. The systems and methods can create pressure waves that radiate outward from the balloon. The systems and methods can create pressure waves that travel beyond the length of the balloon. The systems and methods can create pressure waves that propagate circumferentially and transmurally. The systems and methods can disrupt superficial calcium. The systems and methods can disrupt deep calcium deposits. The systems and methods can create multi-plane fractures. The systems and methods can create micro fractures. The systems and methods can create macro fractures.

In some embodiments, disclosed herein are systems and methods that produce unexpected results clinically that maintain the integrity of the soft tissue. The calcium deposits can include fibrin networks extending through the deposit. There can be disease morphologies that have networks through the calcium sheet. There can be additional structures to the cellular network of the calcium. In some embodiments, the systems and methods can fracture calcium bonds around the fibrin networks while leaving the network intact. The fibrin can act as a scaffold for the calcium. The fibrin can limit or reduce dissections when left intact. The pressure waves generated by the system 1100 can pass through the fibrin, which may have a density similar to soft tissue. The pressure waves generated by the system 1100 can shear the calcium sheet. The calcium fragments can remain adhered to the fibrin network. The calcium fragments can remain within the tissue of the vessel.

In some embodiments, disclosed herein are systems and methods have advantages over other systems. It is noted that some other intravascular devices deliver acoustic shockwaves which may be associated with disadvantages. One known complication of acoustic shockwave treatment can be ventricular ectopic activity. Another known known complication of acoustic shockwave treatment can be ventricular ectopics. In some embodiments, acoustic shockwaves can depolarize cardiac tissue. This complication can be more likely in certain populations. In some embodiments, acoustic shockwaves can result in ventricular arrhythmias. In some embodiments, some other intravascular devices utilize spark-gap technology to generate the acoustic shockwave. One additional complication of acoustic shockwave treatment can be a subsequent increase in surface area initiation sites (nidus) for further calcification. In some embodiments, acoustic shockwaves used to break up calcified tissue can result in blooming calcification in the operation sites. In some embodiments, these disadvantages can be reduced or minimized when the acoustic shockwaves are combined with the serration technology according to the devices and methods described herein.

In some embodiments, disclosed herein are systems and methods that produce unexpected results clinically by related to serration in combination with lithotripsy. The balloon 100 used with the system 1100 can be configured for serratoplasty or serration angioplasty. The balloon 100 used with the system 1100 can be used as a standalone angioplasty balloon. The balloon 100 used with the system 1100 can be a preparation device prior to a follow-on therapy. Follow-on therapies can include but are not limited to: stenting, placing a drug eluting stent, performing atherectomy, high pressure ballooning or drug coated balloon treatment or other endovascular procedures requiring effective luminal preparation. Whether or not the balloon 100 is used as a preparation device for follow-on therapy or a stand-alone therapy, through the use of the raised features 300, the balloon 100 used with the system 1100 can effectively manage plaque or calcium by weakening the bonds, initiating cellular or structural disassociation and minimizing cellular or structural compression, typically generated by angioplasty alone. This mechanism of action using mechanical means to fracture vesical calculi can be termed lithotripsy. Serratoplasty designs including the raised features 300 can be used for lithotripsy using the system 1100. The use of the system 1100 with the balloon 100 in the vasculature can allow for the diseased lumen, including all degrees of calcified vasculature, to be safely expanded and accurately dilated and stretched, using low pressure, to a desired diameter without creating numerous and substantial dissections and elevated flaps. Serratoplasty designs used for lithotripsy can effectively crush the vascular calcium, independent of calcium location, including intimal calcium or medial calcium, or a combination of both intimal and medial calcium, through the combination of the raised features 300 and the pressure wave configured to fracture the calcium features. These fractured vascular calcium elements caused by lithotripsy are more easily crushed through the subsequent or concurrent pressure of the balloon 100. The pressure of the balloon 100 can impart a radial force to tear apart the calcium bonds. The pressure of the balloon 100 can also impart a tangential force to tear apart the calcium bonds. The pressure of the balloon 100 can exert a longitudinal force to tear apart the calcium bonds. Independently, these forces can exert a compounding force, in combination with the serrated lines, that induces energy upon the calcium bonds. The force can break apart the vascular calcium bonds therein enabling a crushing of the vascular calcium into smaller calcium fragments. The smaller calcium fragments are less restrictive to the vasculature. With smaller vascular calcium elements, the vasculature can return to a higher level of vascular compliance. The serrations with lithotripsy can enable the plaque to be dilated more evenly and smoothly and avoid forming random cracks that may lead to dissection and residual stenosis. The plaque, after it has been treated or pre-treated with serration, in some cases, can also be dilated with lower pressure than that which is used in standard balloon angioplasty. The lower intra-balloon pressure (e.g., less than, equal to or combinations of 4, 3.5, 3, 2.5, 2, 1, 0.5 atm) can be applied by the balloon 100 with the raised features 300. The raised features 300 can cause less disruption of the plaque, fewer dissections, and less injury to the wall of the vessel. This low pressure serratoplasty with lithotripsy can be less likely to cause the biological reaction that often follows balloon angioplasty with neointimal hyperplasia or smooth muscle cell replication. In addition, serration with lithotripsy can permit the plaque to expand with less fracturing or disruption of the plaque during balloon angioplasty. By preparing the plaque using the balloon 100 with the raised features 300 capable of serrating the inside of the vessel 10, the number and severity of dissections can be reduced. This can decrease the need for stent placement to be used to treat dissection or residual stenosis after balloon angioplasty with serration and lithotripsy.

The system 1100 used with the balloon 100 can have numerous advantages related to clinical outcomes. The system 1100 used with the balloon 100 can lead to consistently higher flow rates for all sizes of vessel 10. The system 1100 used with the balloon 100 can lead to increased flow rates. The system 1100 used with the balloon 100 can increase flow rates after creating the linear dissected line compared with POBA. While not bound by a theory, POBA can rely on simple expansion of the vessel wall. POBA can increase pressure to force the vessel wall outward. In some embodiments, POBA does not remodel the vessel wall. Rather, the POBA can apply a high-pressure force that physically pushes the diseased wall outward. With POBA, the vessel can recoil when the POBA is removed. This can be shown by the resulting flow rate ratio. While POBA can increase the flow rate through the vessel at least temporarily, the system 1100 used with the balloon 100 can consistently produce a greater flow rate ratio compared with POBA. For any give pre-treatment radius, the system 1100 used with the balloon 100 produces a greater flow rate ratio. The greater flow rate ratio can be greater that POBA by a factor of 2, 3, 4, 5, 6, 7, 8, 9, 10 or any range of the foregoing values. For small pre-treatment radius of the vessel 10, such as under 0.15 mm, the system 1100 used with the balloon 100 can increase flow rate ratio up to a factor of 10 compared with POBA.

The system 1100 used with the balloon 100 can also lead to more predictable results. The flow rate ratio for the system 1100 used with the balloon 100 for all of the data points can be close to the best fit line. The flow rate ratio for POBA can have much more variation in the data points. This variation in data points for POBA can be caused by dissections that increase the diameter unexpectedly. POBA can be more prone to dissection than the system 1100 used with the balloon 100. POBA can create unpredictable fractures due to high pressure. This variation in data points for POBA can be caused by varying degrees of recoil depending on the state of the vessel. POBA can be a simple expansion and contraction. POBA can be more prone to recoil than the system 1100 used with the balloon 100. This variation in data points for POBA can be caused by the temporary nature of POBA. POBA can only support the expanded state of the vessel while the POBA is inflated. In some embodiments, POBA does not remodel the vessel in a predictable and permanent way.

The system 1100 used with the balloon 100 can have much less variation in the data points. This low variance for the system 1100 used with the balloon 100 can be caused by the predictable and repeatable creation of linear dissected lines. The dissected lines can have a narrow width compared to the length. The dissected lines can have a narrow width compared with the circumference of the vessel 10. The dissected lines can have a narrow width thereby disturbing less of the surface of the wall of the vessel 10. The system 1100 used with the balloon 100 can create predictable dissected lines under lower pressure than POBA. This low variance for the system 1100 used with the balloon 100 can be caused by less recoil. The vessel 10 can be less likely to return to a small diameter or cross-section. The dissected lines can provide room for the vessel 10 to expand without disturbing the integrity of the diseased portion or plaque. The dissected lines can provide flexibility for the vessel 10 to expand under the fluid flow. This low variance for the system 1100 used with the balloon 100 can be caused by the permanent nature of linear serration line created by the serrated balloon 100. The system 1100 used with the balloon 100 can permanently change the vessel 10 by the addition of one or more permanent and predictable dissected lines. The system 1100 used with the balloon 100 can remodel the vessel in a permanent and predictable manner. The remodeling can lead to less variation in the data points for the serrated balloon 100.

In some embodiments, disclosed herein are systems and methods that produce unexpected results clinically related to flow rate. For pre-treatment radius of the vessel 10 between 0.05 mm and 0.15 mm, the system 1100 used with the balloon 100 can increase the flow rate ratio post-treatment versus pre-treatment between 1000 and 10000. For pre-treatment radius of the vessel between 0.15 mm and 0.25 mm, the system 1100 used with the balloon 100 can increase the flow rate ratio post-treatment versus pre-treatment between 100 and 1000. For pre-treatment radius of the vessel 10 between 0.25 mm and 0.35 mm, the system 1100 used with the balloon 100 can increase the flow rate ratio post-treatment versus pre-treatment between 100 and 1000. For pre-treatment radius of the vessel 10 between 0.35 mm and 0.45 mm, the system 1100 used with the balloon 100 can increase the flow rate ratio post-treatment versus pre-treatment between 10 and 1000. The system 1100 used with the balloon 100 can produce a higher flow rate ratio compared to POBA. The system 1100 used with the balloon 100 can improve the flow rate post-treatment substantially better than POBA.

In some embodiments, disclosed herein are systems and methods that produce unexpected results while utilizing familiar technology platforms. In some embodiments, the balloon 100 can be sized at 1:1 ratio with the reference diameter of the target vessel to allow for apposition to the wall of the vessel 10. In some embodiments, the balloon 100 can be sized greater than the diameter of the vessel 10, such as 1.1:1 or 1.2:1 up to 1.5:1. The balloon 100 can be taken to a treatment site across the calcified lesion. The balloon 100 can be connected to the system 1100 for generating pressure waves. The balloon 100 can be connected to the inflation device 400 for providing the base pressure. The balloon 100 can be inflated with any fluid medium described herein. The balloon 100 can be inflated with a mixture of saline and contrast (at a ratio example such as a 50/50 ratio). The balloon 100 can be inflated to a sub-nominal pressure of between 1 atm and 4 atm in some embodiments. The pressure waves can be generated to produce the results described herein.

In some embodiments, disclosed herein are systems and methods that produce unexpected results clinically while utilizing proven serration technology. The balloon 100 can be coupled to the strips 200 with the raised features 300. The balloon 100 can be removably coupled to the carrier 1200. The balloon 100 can be used with external structures that produce lines of compression along the intima. The balloon 100 can be used with external structures that can produce serrated lines longitudinally along the lumen of the vessel 10. The serrated lines can effectively aid in separation of materials (similar to serrated lines of paper, stamps, cardboard, granite stone, marble, which aid in separation etc.). Since disease morphology often involves both soft and hard materials, serration can be advantageous to effectively aid in vessel expansion. There can be several ways to produce serrations. For example, a series of raised features 300 can be configured to produce serrations or linear serrated scoring at the deployment site.

In some embodiments, disclosed herein are systems and methods that produce unexpected results clinically related to lumen gain. In some embodiments, the lumen gain from the raised features 300 used with the balloon 100 can be better than the balloon 100 alone. The improvement of lumen gain has been recorded with 49% improvement of final stenosis with raised features 300 used with the balloon 100 compared to the balloon 100 alone. The increased lumen diameter of the vessel 10 can improve flow. Applying Poiseuille's Law to vascular blood flow, the volume flow rate can be calculated. The improvements in the average flow rate ratio can be compared, which can be defined as: (the radius post intervention divided by the radius pre intervention) raised to the 4^{th} power. The data set comparing the balloon 100 to the raised features 300 used with the balloon 100 can be mapped along with a set of power-fitting curves. The raised features 300 used with the balloon 100 can be able to achieve a greater than two times the average flow rate ratio compared with the balloon 100.

In some embodiments, disclosed herein are systems and methods that produce unexpected results clinically related to drug delivery. In some embodiments, the inclusion of the raised features 300 can offer advantages to balloons 100, not only for the preparation of tissue prior to or concurrent with the use of drug coated balloons, but also as a single step drug delivery mechanism. The inclusion of drug coatings on the raised features 300, around the raised features 300, and/or within reservoirs of the raised features 300, or regions neighboring the raised feature 300 on the balloon 100 can facilitate the delivery of the desired drug or other therapeutic agent(s) deeper into the desired target location, such as for example the intima, the media, or the adventitial surface of the vessel 10.

In some embodiments, disclosed herein are systems and methods that produce unexpected results clinically related to creating consistent serrations. The raised features 300 can be a modified cutting member for instance by removing a honed tip in favor of the radially outwardly facing surface 304 which can be unhoned. The raised features 300 can have dimensions that provide for a more flexible, more stable design. The raised feature 300 can serrate or approximate serrations in the tissue. The raised feature 300 of the strip 200 can offer, for example, about or greater than about 8, 9, 10, 11, 12, 13, 14, 15, 20, or more degrees lateral flexion with or without sections where the strip 200 can be less or not serrated. Some embodiments can include a series of raised features 300, either in tandem or with periods of raised features 300 as described elsewhere herein. The strip 200 can have spaces between the raised features 300 or on the ends of the strip 200. The raised features 300 can be divided into multiple discrete sections. The raised features 300 have a length, for example, in the range of 0.01 inch to about 0.10 inch. The raised features 300 can be separated by spaces of, for example, about 0.01 inch to about 0.08 inch. In some embodiments, the length of the raised features 300 can be divided into multiple discrete sections separated by spaces of 1:2, 1:2.5, 1:3, 1:3.5, or 1:4, where the space between the raised features 300 can be larger than the length of the raised features 300. The entire strip 200 may have discrete sections of raised features 300 at any one or any number of locations along the strip 200. Once pressure is applied by the balloon 100 into tissue, the resulting tissue disruption may appear to be a series of dots and dashes or any combination of dots and dashes, produced by the raised features 300 piercing the tissue only in narrow regions where the discrete raised features 300 are located. The effect of this discrete localized penetration is that the balloon 100 used with the raised features 300 produces a new angioplasty effect, referred to as serration or serratoplasty. The raised features 300 can be small raised features, able to produce dot like effects (or serration like features) in tissue. The raised features 300 can be smaller on the ends of the strip 200, then longer raised features 300, able to produce dashes can be in the center portion of the strip 200. The raised features 300 can include a mix small and longer raised features in alternating patterns. The raised features 300 can include a series of dots spaced by one or more dashes with a repeated pattern or a non-repeated pattern. The strip 200 can have 1, 3, 4, 5, 6, or 8 raised features 300 on the outside of the balloon 100 with the strip 200 being typically less than the balloon body length.

In some embodiments, disclosed herein are systems and methods that produce unexpected results clinically related to angioplasty at lower pressures. As described herein, the serrated vessel 10 can open with lower pressure and can be capable of retaining the affective lumen gain over time. Observations have been made post serration angioplasty at 15 minutes and higher with the appearance of the lumen gain being retained. The concept of vessel recoil, or vessel lumen decreasing after being stretched open, can occur and there can be a need for designs capable of overcoming the vessels tendency to recoil. With serration angioplasty, it has been observed clinically that minimal to no recoil has been recorded. To assess for recoil events, physicians have collected angiographic images after serration angioplasty and again 15 minutes after serration angioplasty. The images have been assessed for changes in vessel diameter or recoil and the results were that minimal to no recoil was detected. These observations may not be expected and have encouraged further designs to investigate serration angioplasty embodiments designed for the purpose of reduction in recoil. This reduction in recoil phenomenon can be seen in both the arterial system and the venous system, especially in diseases found in anastomosis or near arterial-venous anastomosis. The balloon 100 used with the raised features 300 has shown effective outcomes for reducing and in some cases eliminating recoil in the arterial system.

In some embodiments, disclosed herein are systems and methods that produce unexpected results clinically related to pre-treatment and subsequent angioplasty at low pressures. It has also been observed that balloons 100 smaller than the diameter of the reference vessel 10 can be used to pre-treat the diseased region. This pre-treatment can provide equally effective serration angioplasty treatment. In some methods, a subsequent balloon angioplasty can be performed, at low balloon pressures of about 4 atmospheres or less due to preparation of the plaque with perforations, so as to avoid injury to the arterial wall or its sub-intimal tissues. By performing plaque preparation and then low-pressure angioplasty, there can be less likelihood of a dissection occurring that generates deep tissue tearing while still potentially exposing the media layer of the artery. Exposure of deep tissue from within the artery wall can in some cases stimulate thrombus formation by collagen exposure and also can stimulate smooth muscle cell growth which later can cause neointimal hyperplastic occlusion of the artery. This decrease in number and also decrease in severity of dissection can in some cases be an advantageous differentiating factor in comparison of the system 1100 to conventional cutting or scoring devices and other forms of plaque disruption including but not limited to angioplasty delivering ultrasonic energy.

In some embodiments, disclosed herein are systems and methods that produce unexpected results clinically related to the impact of the raised features 300. In some embodiments, the raised features 300 can deliver intense mechanical energy. In some embodiments, the motion of the raised features 300 under the influence of the pressure wave can translate or create a shock wave. The contact event between the raised features 300 and the plaque can amplify the effectiveness of the translated or created shock wave. In some embodiments, translation of energy through the strips 200 is independent and distinct from the shock wave at the interface of the leading edge of the raised features 300 and the impact of the calcified lesion. In some embodiments, the shock wave of the raised features 300 may not be an ambient shock wave, such as those created by electrodes within the catheter. In some embodiments, the shock wave may not be an electrical event with a cathode and anode, wherein the load of electrons is seeking a release through an arc event within the liquid medium. Instead, in some embodiments, the shock wave can be generated at the interface of the leading edge of the raised features 300 and the impact of the calcified lesion, such as a contact-to-contact event. The contact between the raised features 300 and the plaque can generate an shock wave. The contact between the raised features 300 and the plaque can utilize intense mechanical energy carried by the shock wave. There can be high energy motion of the raised features 300 against the plaque. The radially outward facing surface 304 of the raised feature 300 can be an impact surface. The motion of the raised feature 300 driven by the pressure wave can build energy. The raised features 300 can hit the plaque. The raised features 300 can unload energy in a shock wave. The raised features 300 can have a mechanical vibration. The mechanical impact of the raised features 300 against the plaque can create a shock wave. The mechanical impact of the raised features 300 can be driven by the pressure wave.

In some embodiments, disclosed herein are systems and methods that produce unexpected results clinically related to fracturing calcium. The impedance of the plaque can be similar to the impedance of other calcium structures. The plaque can have high density, especially hardened plaque. The plaque can strongly reflect sounds waves. The plaque can therefore be difficult to break up with sound waves alone in some embodiments, to further aid the energy of sound waves, the system 1100 can utilize mechanical impacts of the raised features 300 driven by the pressure waves in some embodiments. The mechanical impacts can reduce reflection compared with sound waves. The mechanical impacts can be more effectively absorbed by the plaque compared with sound waves. The plaque can be altered by mechanical contact of the raised features 300 and the strips 200. The mechanical impacts can have a lithotripsy effect. The mechanical impacts can be applied at a high frequency at acoustic wave speeds. In some embodiments, the mechanical impacts can be applied at a high frequency at speeds slower than acoustic wave transmission speeds. The mechanical impacts can create a pulse of the raised features 300 that move in and out of the plaque. The mechanical impacts can create a pulse of the raised features 300 at a higher amplitude than the amplitude of the pressure wave. The mechanical impacts can create a pulse of the raised features 300 that creates an impaction force. The mechanical impacts can have an atherectomy effect. The mechanical impacts can be delivered at a lower frequency than the frequency of the pressure wave. The mechanical impacts can create a pulse that moves along the plaque longitudinally. The mechanical energy can create a pulse at a lower amplitude than the amplitude of the pressure wave. The mechanical impacts can create a pulse that scrubs the surface of the plaque. The mechanical impacts can have components that move the strip 200 in both a tangential and longitudinal orientation relative to the tissue surface. The mechanical impacts can move the strip 200 in and out of the tissue. The mechanical impacts can move the strip 200 longitudinally along the tissues surface along the longitudinal axis of the strip 200. The mechanical impacts can move the strip 200 both in and out or the tissue and longitudinally along the axis of the strip 200.

In some embodiments, disclosed herein are systems and methods that produce unexpected results clinically related the hammering motion. The system 1100 can utilize the hammering motion. The balloon 100 can be pulsated by the pressure waves to create a hammering effect. The raised features 300 can be pulsated by the pressure waves to function as a percussive drill. The strip 200 can be pulsated by the pressure waves to function as a percussive drill. The system 1100 can break through hardened materials like plaque. The system 1100 can use pulsations to repeatedly strike the plaque with the raised features 300. The system 1100 can fracture the plaque with mechanical impacts. The system 1100 can create a hole or perforation with the raised features 300. The percussive motion of the balloon 100 can facilitate formation of the perforations in the plaque. The percussive motion can shorten the time to form the perforation. The raised features 300 can repeatedly impact the plaque. In some embodiments, the raised features 300 can repeatedly impact the plaque without rotation of the balloon 100. In some embodiments, the raised features 300 can repeatedly impact the plaque without longitudinal movement of the balloon 100. In some embodiments, the ballon 100 can stay in the same location within the vessel as the balloon 100 pulsates by the pressure waves. The balloon 100 can be particularly suited for impacting plaque, which can be a dense, hard material. The percussive motion can facilitate penetration of plaque, which can be difficult to penetrate with other methods.

In some embodiments, disclosed herein are systems and methods that produce unexpected results clinically related to the radial direction of motion. In some embodiments, the pulsations of the balloon 100 by the pressure waves can have a direction. In some embodiments, the raised features 300 can pulse radially outward. The raised features 300 can pulse toward and away from the plaque. The raised features 300 can pulse toward the vessel wall. The balloon 100 can expand and contract in diameter with each pulse. The balloon 100 can expand radially outward. The balloon 100 can expand to increase and decrease the diameter. The balloon 100 can expand radially. The raised features 300 can diverge from a center of the balloon. The raised features 300 can pulse around a central axis of the balloon 100. The raised features 300 can move toward and away from a central axis of the balloon 100. The raised features 300 can pulse deeper into the plaque. The raised features 300 can pulse toward the intima layer. The raised features 300 can pulse toward the medial layer. The raised features 300 can pulse toward the adventitia. The raised features 300 can pulse toward outward layers of the vessel. The raised features 300 can drive deeper into the plaque. The raised features 300 can create a mechanical impact that propagates radially. The raised features 300 can create a line of weakness that quickly propagates to provide rapid and sustainable luminal gain.

In some embodiments, disclosed herein are systems and methods that produce unexpected results clinically related to the longitudinal direction of motion. In some embodiments, the raised features 300 can pulse longitudinally with the pressure waves. The raised features 300 can pulse across the plaque. The raised features 300 can pulse lengthwise. In some embodiments, the balloon 100 can lengthen with the pressure waves. In some embodiments, the balloon 100 can pulse longitudinally with the pressure waves. The balloon 100 can pulse across the plaque. The balloon 100 pulse lengthwise. The ballon 100 can expand distally. The balloon 100 can expand proximally. . . . The balloon 100 can expand and contract in length with each pulse. The balloon 100 can expand longitudinally with the pressure waves. The balloon 100 can expand to increase and decrease the length. The balloon 100 can expand in the lengthwise dimension. The raised features 300 can remain at the same position from the central axis of the balloon 100. The raised features 300 can remain at the same depth within the plaque. The raised features 300 can move toward and away from other perforations in the plaque. The raised features 300 can move toward holes created by other raised features 300. The raised features 300 can create have micromotion relative to the plaque. The raised features 300 can scrape the plaque. The raised features 300 can rub or grind against the plaque. The raised features 300 can create a wave that propagates longitudinally.

In some embodiments, disclosed herein are systems and methods that produce unexpected results clinically related to the radial and longitudinal direction of motion. The raised features 300 can pulse radially and longitudinally. The balloon 100 can expand radially and longitudinally. The balloon 100 can expand and contract in diameter with each pulse. The balloon 100 can expand and contract in length with each pulse. The raised features 300 can pulse periodically. The raised features 300 can create a shock pulse. The raised features 300 can create a mechanical impact that propagates radially and longitudinally.

In some embodiments, disclosed herein are systems and methods that produce unexpected results clinically related to the change in calcium. In some embodiments, the pulsations by the pressure waves can create changes in the plaque. The pulsations can create disturbances that alter the chemical or biological bonds of the plaque. The pulsations can break calcium bonds. In some embodiments, breaking the bonds of calcium can require adding energy. The calcium bonds of plaque, as measured by the bond energy, can be strong. For example, it takes 80 kcal/mol to break a C—C bond, and 145 kcal/mol for a C═C bond. The system 1100 can utilize mechanical energy that can be greater than 145 kcal/mol to break calcium bonds. The pressure waves can create a shearing force on the calcium sheets. The pressure wave can act on calcium sheets, which are dense deposits of calcium. The calcium sheets can be circumferential. The pressure waves can create microfractures in the calcium. The pressure waves can spare surrounding soft tissue. The pressure waves interact with the calcium which is denser than soft tissue. The microfractures can improve vessel compliance. The microfractures can allow for circumferential expansion. The microfractures can facilitate crack propagation along the serrations.

In some embodiments, disclosed herein are systems and methods that produce unexpected results clinically related to balloon growth. As described herein, balloon growth can be directly correlated to the pressure applied to the balloon 100. The pressure can be applied by the inflation device 400. The pressure can be applied by the generator 800. In some embodiments, the balloon 100 can grow at a scale that is uniform as the pressure increases. In some embodiments, the balloon expansion can be approximately 1% radius growth per ATM of pressure. In some embodiments, the balloon growth can be 2% to 5% increase in radius per ATM of pressure. In some embodiments, the balloon growth can be between 6% and 10% per ATM pressure. Balloon growth can be associated with extensions or contraction in the diameter, however it can be also associated with growth in the length of the balloon 199. The pressure applied to the surface of the vessel can be typically dependent on the pressure applied to the ballon 100 and independent of the balloon growth. In some embodiments, the balloon expansion can be minimized or controlled to limit over expansion while delivering the pressure, forcing the lesion to yield under the pressure load.

In some embodiments, disclosed herein are systems and methods that improve over angioplasty balloons without raised features 300. It has been observed in thousands of cases that the use of serration enhanced angioplasty with raised features 300 applied to diseased lesions yield at lower pressures than angioplasty without raised features 300. Additionally, it has been observed that as the pressure increases and the diseased vessel yields, the balloon diameter can further increase allowing further expansion of the vessel along the serrated lines. The mechanism of action of serration angioplasty can be described herein. Many of the embodiments described herein offer an advantage in the expansion of diseased vessels over both plain balloon angioplasty without raised features 300.

In some embodiments, disclosed herein are systems and methods that improve over serration over serration angioplasty without wave generation The pulse wave serration angioplasty has several advantages described herein. The embodiments described herein incorporate the use of pressure waves to the balloon body thereby these embodiments can leverage an array of advantages offered by combining serration balloon angioplasty with pressure wave generation. These advantages can include driving force of the raised features 300 in and out of the tissue, reducing the time for lesion yielding, minimizing neighboring tissue damage, and increasing vessel compliance by crushing bonds of rock hard vascular disease. Embodiments can produce an in and out motion in the radial direction of the raised features 300, in some embodiments. Embodiments can produce an in and out motion in the longitudinal direction of the raised features 300, in some embodiments. In some embodiments, these advantages can be achieved by using the balloon expansion ratio.

The embodiments can provide various systems and methods for pulse wave enhanced balloon angioplasty treatments that overcome possible disadvantages associated with conventional balloon angioplasty treatments for any vessel in the body. In some embodiments, the treatments can include directing a constant balloon pressure of the balloon 100 toward a diseased vessel. In some embodiments, the pressure wave can include an acoustic wave. The acoustic wave can propagate through the entire vessel in contact with the balloon 100, including the intima, media, and advantitia of the vessel. The acoustic wave can propagate from the site of impact of the raised features 300. The acoustic wave can propagate beyond the length of the balloon 100. The acoustic wave can be effective at cracking, crushing, or fracturing substantially all rigid or tightly bound features within the surrounding vessel walls. The system 1100 can synergistically improve the efficiency of the pressure wave and improves the efficiency of the strips 200 with the raised features 300. In some embodiments, the strips 200 with the raised features 300 can be a set of single strips that are placed longitudinally on the balloon 100. In some embodiments, the strips 200 with the raised features 300 can be a set of single strips that are placed in an array that is circumferentially oriented where one or more strips are placed longitudinally. For instance, referring to the cross-sectional view as a clock, the strips 200 can be placed at 12'oclock, at 3'oclock, at 6'oclock, and at 9'oclock around the circumference of the balloon 100 in some embodiments. In some embodiments, the strips might only be at 12'oclock and 6'oclock around the circumference of the balloon 100. In some embodiments, the strips can be placed at 12'oclock, 4'oclock, and 8'oclock around the circumference of the balloon 100. In some embodiments, a single strip can be utilized and placed at 12'oclock. Other variations are contemplated.

The combination of strips 200 with the raised features 300 with pressure wave generation can produce a repetitive surface impact generating an increase in likelihood of weakening the cellular bonds that are restricting the inner diameter of the vessel.

The system 1100 can allow for control of various aspects of the pressure wave. The system 1100 can control impact frequency. The system 1100 can control impact frequency by controlling the speed of the motor 830. The system 1100 can control the amplitude of the pressure change. The system 1100 can control the amplitude of the pressure change by adjusting the compression of the spring 846. The system 1100 can control the amplitude of the pressure change by creating stops or limits for the movement of the adjuster 822. The system 1100 can control the delivery of the pressure waves. The system 1100 can control the delivery of the pressure waves by turning on and off the motor 830 with the button 820.

The system 1100 can be a modular, hand-held medical instrument. The system can produce pressure waves in a cost-effective manner. The system 1100 can have a mechanical mode of operation which causes the impact of the raised features 300 against plaque. The system 1100 can be robust by having a durable and resistant housing. The system 1100 can be low-maintenance and reliable by having few moving parts. The system 1100 can supply a precise impact energy that is repeatable by the generation of pressure waves by the generator 800. The system 1100 can fracture calcifications embedded tissue. The generator can produce calcium fracturing by generating the pressure wave. The system 1100 can include one or more fracture producing components that are located within the proximal handle of the generator 800. The fracture producing components can produce a pressure wave that fractures calcifications within tissue. The fracture producing components can mechanically produce an impact force which is converted to pressure wave within the liquid medium. The pressure wave can be transmitted from the proximal end of the system 1100 to the distal end of the balloon 100 in some embodiments. The pressure wave can be transmitted from the reservoir 854 in the proximal handle to the balloon 100. The pressure wave can be propagated through the balloon 100 and the strips 200 with the raised features 300 to fracture calcifications. The system 1100 can fracture calcium without removing calcium from the vessel. The calcium can remain with the vessel layers. The calcium can remain adhered to the vessel wall. The system 1100 can modify vascular calcium.

The system 1100 can create a mechanical impact within the vessel. The lobes 120 of the balloon 100 can be in contact with the vessel wall. The raised features 300 of the strip 200 can be in contact with the vessel wall. The vessel wall can include plaque or hardened regions. The mechanical impact can be created by a rapid outward and inward motion of the raised features 300 of the strip 200. The mechanical impact can be created by outward motion that follows a fluid wave of the pressure wave. The mechanical impact can be created by outward motion that propagates distally. The mechanical impact can be created by a rapid outward and inward motion that is greater than the height of the raised feature 300. The mechanical impact can be created by a rapid outward and inward motion that is greater than the height of the strip 200. The mechanical impact can be created by an outward motion that engages the vessel wall with the raised features 300. The mechanical impact can be created by inward motion that disengages the raised features 300 from the vessel wall. In some embodiments, the raised features 300 of the strip 200 can have vibratory motion. In some embodiments, the amplitude of the movement of the raised features 300 of the strip 200 can be on the order of millimeters. In some embodiments, the amplitude of the movement of the raised features 300 of the strip 200 can be on the order of micrometers. In some embodiments, the amplitude of the movement of the raised features 300 of the strip 200 can be less than the height of the raised features 300. In some embodiments, the amplitude of the movement of the raised features 300 of the strip 200 can equal to the height of the raised features 300. In some embodiments, the amplitude of the movement of the raised features 300 of the strip 200 can be greater the height of the raised features 300. In some embodiments, the pressure wave can cause movement of the strip 200 with the raised features 300 of less than 0.05 mm, 0.05 mm, greater than 0.05 mm, less than 0.1 mm, 0.1 mm, greater than 0.1 mm, less than 0.15 mm, 0.15 mm, greater than 0.15 mm, less than 0.2 mm, 0.2 mm, greater than 0.2 mm, less than 0.25 mm, 0.25 mm, greater than 0.25 mm, or any range of two of the foregoing values. In some embodiments, the pressure wave can cause movement of the strip 200 less than the height of the strip 200, the height of the strip 200, greater than the height of the strip 200, twice the height of the strip 200, greater than twice the height of the strip 200, three times the height of the strip 200, greater than three times the height of the strip 200, or any range of two of the foregoing values.

The system 1100 can create pressure waves that travel through the vessel wall. The pressure wave can include an acoustic waves that emanates from the balloon 100. The system 1100 can create pressure waves that travel through soft vascular tissue. The system 1100 can create pressure waves that travel through highly calcified regions. The system 1100 can create pressure waves that break calcium bonds. The system 1100 can create pressure waves that reduce the thickness of calcium deposits. The system 1100 can create pressure waves that reduce the thickness of plaque. The system 1100 can create pressure waves that allow the raised features 300 to penetrate less thickness. The system 1100 can create pressure waves that allow the raised features 300 to penetrate through less material. The system 1100 can create pressure waves that reduce the layer of calcium deposits thereby decreasing the height to the medial layer. The system 1100 can create pressure waves that reduce the layer of calcium deposits thereby decreasing the height of the crack. The system 1100 can create pressure waves that effectively manages calcium by weakening the bonds. The system 1100 can initiate cellular or structural disassociation. The system 1100 can minimize cellular or structural compression.

The system 1100 can create pressure waves that facilitate crack propagation. The system 1100 can create linear cracks can be a key structural alteration in the vessel. The system 1100 can create a linear crack that extends past the intima layer. The system 1100 can create a linear crack that extends past the medial layer. The system 1100 can have raised features 300 which only penetrate the intima in some methods. The system 1100 can have raised features 300 which only penetrate the media in some methods. The system 1100 can form a crack through crack propagation along a series of microperforations. The system 1100 can form a crack which only disturbs a small portion of the circumference of the vessel wall. The pressure wave can allow the crack to open more uniformly along strip 200. The pressure wave can cause the crack to open at the wave speed.

The system 1100 can delivers peak pressure pulses of the pressure wave which apply a compressive force on calcium. In some embodiments, the system 1100 does not include any major thermal rise due to the low intensity of energy being delivered. In some embodiments, the system 1100 has no distinguishing thermal components such as arc generators, mitigating any risk of thermal injury. The system 1100 can allow for longitudinal treatment of the vessel along the length of the balloon 100. The system 1100 can create a pressure wave that radiates circumferentially. The system 1100 can create a pressure wave that radiates transmurally. The system 1100 can create a pressure wave that radiates in a focused manner. The pressure wave can be focused by the raised features 300 of the strip 200. The pressure wave can impart a compressive force on calcium. The pressure wave can fracture the calcium without pulverization. The penetration depth of the pressure wave can be 1 mm, 2 mm, 3 mm, 4 mm, 5 mm, 6 mm, 7 mm, 8 mm, 9 mm, 10 mm, or any range of two of the foregoing values. The primary mechanism for calcium fracturing can be compression stress provided by the pressure wave. The primary mechanism for calcium fracturing can be tensile stress provided by the pressure wave. The pressure induced by the pressure wave can be consistent along the length of the balloon 100. The system 1100 can disrupt calcium deposits located near the surface of vessel wall. The system 1100 can disrupt calcium deposits located within the vessel.

The balloon 100 can be inflated to a low pressure, such as 2 atm. The liquid medium can be saline and/or contrast. The low pressure can be sufficient for the balloon 100 to contact the vessel wall. The balloon 100 can be in contact with the vessel wall for transfer of the pressure wave. The balloon 100 can be inflated to a diameter greater than the initial vessel diameter to produce adequate contact between the balloon 100 and the vessel wall. The pressure waves can be propagated when the balloon 100 is at least partially inflated. The pressure waves can be propagated when the balloon 100 is inflated to the base pressure. The pressure waves can be propagated when the balloon 100 apposes the vessel wall. The pressure waves can impact superficial calcium, such as plaque adhered to the vessel wall. The pressure wave can impact deep calcium.

In some embodiments, the system 1100 can be limited to longitudinal calcium fractures. The system 1100 can be used to create microperforations without the emission of pressure waves. The microperforations can be created by the raised features 300. In some embodiments, the system 1100 can deliver pressure waves only after the microperforations are created. In some embodiments, the microperforations can provide nucleation points for further energy delivery. The system 1100 can create longitudinal calcium fractures along the microperforations. The pressure waves can be focused to follow the longitudinal orientation of the microperforations. The pressure waves can be focused by the raised features 300. The pressure waves can be focused along longitudinal lines of the strip 200. The pressure waves can be focused along the axis of the vessel. The strip 200 and the raised features 300 can be used to concentrate the energy delivered by the pressure waves due in part to the surface area of the raised features 300. The pressure waves can be delivered primarily along the strips 200. The pressure waves can be magnified by the strip 200. The pressure waves can be magnified due to the material of the strip 200. The pressure waves can be focused due to the shape of the strip 200 and the raised features 300. In some embodiments, the pressure waves do not cause circumferential calcium fractures. In some embodiments, the pressure waves do not cause transverse calcium fractures. In some embodiments, the pressure waves can only cause longitudinal calcium fractures. In some embodiments, the pressure waves can only cause longitudinal calcium fractures of superficial calcium that traverses the luminal surface. In some embodiments, the pressure waves can only cause longitudinal calcium fractures of plaque along the vessel wall. In some embodiments, the pressure waves can cause circumferential calcium fractures of calcium within the layers of the vessel. In some embodiments, the pressure waves can cause microfractures of calcium in deep layers of the vessel. In some embodiments, the pressure waves can only travel along the cleavage planes of the cracks.

In some embodiments, the system 1100 can be a single use intravascular device. The system 1100 can be provided to the user in a sterile state. In some embodiments, the system 1100 can comprise a multiple-use intravascular device. In some embodiments, the generator 800 is reusable. In some embodiments, the catheter 600 and the balloon 100 are disposable. In some embodiments, the system 1100 can be sterilized. In some embodiments, the catheter 600 and the balloon 100 can be sterilized. The system 1100 can be disposable. The system 1100 can be reused for multiple balloons 100 within the same procedure and for the same patient.

In some embodiments, the raised features 300 described herein can offer advantages related to the method of use. The raised features 300 can be disposed on the balloon 100. The raised features 300 can be disposed on the carrier 1200. The raised features 300 can contact the vessel wall at low pressure of the balloon 100. The balloon 100 can be inflated such that the raised features 300 abut the vessel wall. The balloon 100 can be inflated such that the raised features 300 abut plaque on the vessel wall. The balloon 100 can be inflated such that the raised features 300 do not penetrate the vessel wall. The balloon 100 can be inflated such that the raised features 300 do not penetrate plaque on the vessel wall. The generator 800 can rapidly spike the pressure by deflecting the membrane 884. The generator 800 can create a pressure wave. The pressure wave can travel the length of the catheter 600 to the balloon 100. The pressure wave can originate outside of the balloon 100. The pressure wave can originate outside of the catheter 600. The pressure wave can originate within the generator 800. The pressure wave can rapidly inflate and deflate the balloon 100. The pressure wave can rapidly spike the pressure in the balloon 100. The pressure wave can cause the raised features 300 to penetrate the vessel wall. The pressure wave can cause the raised features 300 to penetrate plaque on the vessel wall. The pressure wave can decrease the time to create microperforations. The pressure wave can decrease the base pressure needed to create microperforations. The pressure wave can create the spike in pressure to allow the raised features to penetrate. The raised features 300 can move by the spike in pressure of the pressure wave. The raised features 300 can move by the pressure wave. The pressure wave can cause the raised features 300 to pierce through plaque and produce microperforations. The pressure wave can cause the raised features 300 to pierce through the internal elastic lamina and produce microperforations. The pressure wave can cause the raised features 300 to pierce through the intimal layer. The pressure wave can cause the raised features 300 to reduce recoil. In some embodiments, the raised features 300 can only produce the initial microperforation. In some embodiments, the raised features 300 does not produce the longitudinal crack. In some embodiments, the crack propagates beyond the raised feature toward the medial layer. In some embodiments, the crack propagates between the microperforations. The pressure wave can cause expansion of the balloon 100. The pressure wave can cause an increase in pressure in the balloon 100. The balloon 100 can move by the spike in pressure of the pressure wave. The balloon 100 can move by the pressure wave to apply a force. The pressure wave can cause the balloon 100 to apply a force to the vessel. In some embodiments, the crack propagates due to the force applied by the balloon 100.

In some embodiments, disclosed herein are systems and methods that produce unexpected results clinically related to the low base pressure and the peak positive pressure. In some embodiments, the system 1100 utilizes a base pressure. The base pressure can be a low pressure, such as 1 ATM, 2 ATM, 3 ATM, 4 ATM, or any pressure described herein. In some embodiments, the system 1100 utilizes a pressure wave with a peak pressure. In some embodiments, maximum peak pressure of the pressure wave as low as 10 atm have been effective at creating longitudinal cracks. In some embodiments, maximum peak pressure of the pressure wave as low as 8 atm have been effective at creating longitudinal cracks. In some embodiments, maximum peak pressure of the pressure wave as low as 10 atm have been able to create a lithotripsy effect. In some embodiments, maximum peak pressure of the pressure wave as low as 10 atm have been able to create a shock wave. In some embodiments, the use of a maximum peak pressure of the pressure wave less than 10 atm minimizes vessel injury. In some embodiments, the use of a maximum peak pressure of the pressure wave less than 10 atm enables smoother post-angioplasty surfaces.

In some embodiments, the system 1100 described herein can offer advantages related to crack formation. The pressure wave can induce strain on the wall of the vessel. As the pressure spikes, the tissue seeks weakened areas to release the strain. The pressure wave can cause linear cracks through the release of energy along the microperforations. The depth of penetration can be controlled by the parameters of the pressure wave. The depth of penetration can be controlled by limiting the maximum pressure. The pressure wave can generate a tensile stress that is normal to the plane of the crack. The pressure wave can create shear stress acting parallel to the plane of the crack. The pressure wave can create shear stress acting perpendicular to the crack front. The pressure wave can create shear stress acting parallel to the crack front. The pressure wave can create cleavage planes into the media layer of the vessel wall. The pressure wave can expand the cleavage planes. The pressure wave can create a cleavage plane having a depth of 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, or any range of two of the foregoing values. The pressure wave can accelerate crack formation. The pressure wave can cause the advancing crack plane to migrate beyond the surface of the raised features 300 such that the raised features 300 no longer contact the leading edge of the cleavage plane.

In some embodiments, the system 1100 described herein can offer advantages related to longitudinal crack formation. In some embodiments, the system 1100 can only create longitudinal cracks in the superficial plaque. The longitudinal cracks can be advantageous for vessel expansion. The longitudinal cracks can reduce dissection rates. The longitudinal cracks can permit relaxation of the superficial plaque. The longitudinal cracks can allow dilatation at low angioplasty pressures. The longitudinal cracks can reduce flow limiting dissections. The pressure wave can create longitudinal cracks in plaque. The pressure wave can create longitudinal cracks in hardened calcified regions. The pressure wave can create longitudinal cracks in soft calcified regions.

In some embodiments, disclosed herein are systems and methods that produce unexpected results clinically related to the cleave plane. In some embodiments, the system 1100 described herein can allow for the precise control of the beginning and the end of the cleavage plane. The cleavage planes can propagate based in part on the pressure wave generated by the system 1100. The pressure wave can be adjusted by the user to control the beginning of the cleavage plane. The pressure wave can be adjusted by the user to control the end of the cleavage plane. The cleavage planes can propagate through the internal elastic lamina. The cleavage planes can propagate at least partially through the intima. The cleavage planes can propagate through the intima. The cleavage planes can propagate at least partially through the media. The cleavage planes can propagate through the media. The cleavage planes can propagate through at least some of the layers of the internal elastic lamina, the intima, and/or the media. In some methods, the crack growth can stop before reaching the external elastic lamina. In some methods, the crack growth can stop before reaching the adventitia. In some methods, the crack growth can stop before extending through the adventitia. The crack propagation can cease due to the limited driving force from the pressure wave. The pressure wave can control where the cleavage plane stops within the tissue. The crack propagation can cease due to the small depth of penetration of the raised features 300 that initiate the crack. The crack propagation can cease due to the changing makeup of the layers. The crack propagation can cease due to the increase resistance from layers. The crack propagation can cease due to the parameters of the pressure wave generated.

In some embodiments, disclosed herein are systems and methods that produce unexpected results clinically related to the crack propagation. In some embodiments, the system 1100 described herein can allow for rapid crack propagation. The crack propagation can occur over a short period of time with the pressure waves. The crack propagation can occur over 5 nanoseconds, 10 nanoseconds, 15 nanoseconds, 20 nanoseconds, 25 nanoseconds, 30 nanoseconds, 35 nanoseconds, 40 nanoseconds, 45 nanoseconds, 50 nanoseconds, 55 nanoseconds, 60 nanoseconds, 70 nanoseconds, 80 nanoseconds, 90 nanoseconds, 100 nanoseconds, 200 nanoseconds, 300 nanoseconds, or any range of two of the foregoing values. The pressure wave can accelerate crack formation. The use of the pressure wave can accelerate crack propagation compared to serration technology without the pressure wave. The pressure wave can accelerate crack formation by at least 2×, 10×, 20×, 30×, 40×, 50×, 60×, 70×, 80×, 90×, 100×, 200×, 300×, 400×, 500×, 1000×, or any range of two of the foregoing values compared to serration technology without the pressure wave. As one example, the time reduction has been recorded in experiments to drop from over 120 seconds to under 2 seconds to generate effective calcium fracturing.

The crack propagation can occur rapidly. The pressure wave can reduce the time of vessel occlusion by the balloon 100. The pressure wave can reduce the time needed to obstruct the blood flow. The system 1100 can be used in areas of the body where the vessel can only be occluded for a short period of time. The pressure wave can decrease the time to yield the diseased lesion. The pressure wave can decrease the time to propagate the crack. The system 1100 can increase lumen gain, volumetric blood flow, and vessel compliance compared to systems without pressure waves. The system 1100 can decrease vessel injury and vessel recoil compared to systems without pressure waves.

In some embodiments, disclosed herein are systems and methods that produce unexpected results clinically related to the change in structure of the vessel. In some embodiments, the system 1100 described herein can create smoother arterial surface. The pressure wave can compress plaque. The pressure wave can compress plaque while leaving the plaque adhered to the vessel wall. The pressure wave can compress calcium deposits within the tissue of the vessel. The pressure wave can compress calcium deposits while leaving the calcium deposits within the tissue of the vessel. The pressure wave can compact the plaque against the vessel wall. The pressure wave can break calcium bonds, thereby allowing the plaque to compress under the pressure of the balloon 100. The pressure wave can prevent dissections from flapping within the flow. The pressure wave can create a smooth surface. The pressure wave can stabilize the plaque. The pressure wave can compress dissections against the vessel wall. The pressure wave can reduce the need for stenting. The pressure wave can create a smooth laminal flow surface.

In some embodiments, disclosed herein are systems and methods that produce unexpected results clinically related to origin of the pressure wave. In some embodiments, the system 1100 described herein can produce a pressure wave that originates outside of the balloon 100. In some embodiments, the system 1100 described herein can produce a pressure wave that originates proximal to the balloon 100. The pressure wave can travel the length of the catheter 600 between the reservoir 854 and the balloon 100. In some embodiments, the pressure wave does not dissipate along the length of the catheter 600. The pressure wave can travel nearly instantaneously.

In some embodiments, the system 1100 described herein can produce a pressure wave by the generator 800 separate from the inflation device 400. The inflation device 400 can be a pump for inflation of the balloon 100. The inflation device 400 can inflate the balloon 100 to the base pressure, such as 2 atm. The inflation device 400 can inflate the balloon 100 by pressurization of a fluid medium. The inflation device 400 can be connected to the reservoir 854 via the connector 856. The inflation device 400 can be any inflation system capable of producing a pressure within the balloon 100. The inflation device 400 can inflate the balloon 100 such that the balloon 100 can engage the wall of the vessel. In some embodiments, the pressure of the balloon 100 provided by the inflation device 400 can be insufficient to allow the raised features 300 to create microperforations. In some embodiments, the pressure of the balloon 100 provided by the inflation device 400 can be insufficient to create crack propagation. In some embodiments, the pressure of the balloon 100 provided by the inflation device 400 can be insufficient to create a lithotripsy effect. In some embodiments, the pressure of the balloon 100 provided by the inflation device 400 can be insufficient to break calcium deposits. In some embodiments, the generator 800 can also produce a pressure within the balloon 100. The generator 800 can produce a specific pressure spike via the pressure wave. In some embodiments, the pressure wave produced by the generator 800 can cause the raised features 300 to create microperforations. In some embodiments, the pressure wave produced by the generator 800 can cause crack propagation. In some embodiments, the pressure wave produced by the generator 800 can create a lithotripsy effect. In some embodiments, the pressure wave can be a high energy pulse. In some embodiments, the pressure wave can be a single pressure pulse. In some embodiments, the pressure wave can be a continuous oscillation of pressure. In some embodiments, the pressure wave can have a lower frequency between 1 Hz and 3 Hz. In some embodiments, the pressure wave can have a high intensity of up to 10 atm. In some embodiments, the pressure wave can create a mechanical change in the structure of the calcium, by breaking bonds of the calcium. In some embodiments, the pressure wave can break down calcium deposits within the tissue. In some embodiments, the pressure wave can create longitudinal cracks on superficial plaque. In some embodiments, the pressure wave can produce lumen gain by segmenting the vessel along longitudinal cracks. In some embodiments, the pressure wave can generate almost no heat. In some embodiments, the pressure wave can generate almost no noise.

In some embodiments, the system 1100 described herein can deliver pressure waves to an incompressible fluid. The pressure waves can be generated by the deflection of the membrane 884 which acts on the incompressible fluid in the reservoir 854. The pressure waves can travel from the reservoir 854 to the balloon 100. The pressure waves can be an efficient way to transmit energy almost instantaneously. The pressure waves can deliver a controlled amount of energy. The pressure waves can be controlled by modifying the amplitude of the pressure waves. The pressure waves can be controlled by modifying the compression of the spring 846 which modifies the maximum pressure of the pressure waves. The pressure waves can be controlled by modifying the amplitude of the pressure waves. The pressure waves can be controlled by modifying the speed of the motor 830 which modifies the frequency of the pressure waves. The pressure waves can be controlled by the cam surface 850 of the cam hammer 848. The pressure waves can be controlled by the ramp 864 of the cam can insert 862. The pressure waves can be controlled by the translation distance of the cam hammer 848. The pressure waves can be controlled by the translation distance of the piston 878. The pressure waves can be controlled by the deflection distance of the membrane 884.

To achieve the effectiveness of pulse wave serration angioplasty, the embodiments herein can be based on a theory from percussion drilling. As described herein, five time scales can be defined to aid in understanding the dynamics of pulse wave serration angioplasty.

These time scales can include T1, which can be the travel time of an elastic perturbation of the pressure wave through the body of the balloon 100 to the strip 200 and the individual raised features 300 acting as a linear series of drill bits in an assembly. These time scales can include T2, which can be the period of free vibrations of the strip 200 and the individual raised features 300 acting as combined drill bits and bit/rock interface. These time scales can include T3, which can be the length and intensity of the wave pulse generated in the generator 800 impacting the serrated strip 200 and the individual raised features 300. These time scales can include T4, which can be the response time of the bit/rock interface to an impulse load delivered by the wave pulse generated in the generator 800 to the serrated strip 200 and the raised features 300. These time scales can include T5, which can be the travel time of an elastic perturbation generated as an opposing reaction at the contact of the radially outwardly facing surface 304 of the raised features 300 and the hardened diseased vessel.

The ratios of these time scales can include T1/T2, wherein this ratio characterizes the time scale separation between wave propagation and penetration of the raised features 300 into the diseased tissue. The ratios of these time scales can include T3/T4, wherein this ratio can be a measurement of the length of the pressure wave pulse relative to the response time of the bit/rock interface. The ratios of these time scales can include T1/T3, wherein this ratio compares the travel time of the individual raised features 300 to the length of the pressure wave pulse. The ratios of these time scales can include T5/T3, wherein this ratio reflects the elastic perturbation of the raised features 300 and its impact on the incident wave pulse shape.

These ratios can help in understanding and optimizing the energy transfer efficiency for the system 1100. In addition to the time scales, the stiffness of the material of the strip 200 and corresponding raised features 300, particularly the pseudo-stiffness K at the Bit/Rock Interface (BRI), can play an important role in determining the efficiency of energy transfer during the pressure wave generation. The role of metal stiffness can be outlined in these four parameters. The parameters can include pseudo-Stiffness (K), wherein this parameter characterizes the resistance of the diseased vessel to be penetrated by the raised features 300. Pseudo-stiffness (K) can be a measure of how much force is required to achieve a certain amount of penetration into the diseased vessel. A higher pseudo-stiffness indicates that the diseased vessel can be harder to penetrate, requiring more force for the same amount of penetration. The parameters can include Force-Penetration (F-P) Response, wherein the Bit/Rock Interface (BRI) can be modeled using a bi-linear F-P response, where the loading branch can be characterized by the pseudo-stiffness K. This means that during the initial penetration by the raised features 300, the force required increases linearly with the penetration depth, up to a certain point. The parameters can include energy transfer efficiency, wherein the efficiency of energy transfer from the serrated strip 200 and the radially outwardly facing surface 304 of the raised features 300 to the diseased vessel can be influenced by the pseudo-stiffness K. If K is too high, the raised features 300 may not penetrate effectively, and much of the impact energy may be reflected back rather than being used to penetrate and break or crush the disease in the vessel's cellular matrix. Conversely, if K is too low, the raised features 300 may penetrate too easily, thereby the energy transfer can be dramatically reduced due to insufficient resistance to vessel expansion.

The parameters can include optimal stiffness, wherein there can be an optimal range of pseudo-stiffness K that maximizes energy transfer efficiency. This optimal stiffness can ensure that the raised features 300 penetrate and break or crush the disease effectively, while maximizing the amount of impact energy used to achieve penetration and complex inter-cellular fracturing and crushing to produce smaller particles, rather than being lost to reflections or other inefficiencies.

In some embodiments, the material stiffness at the Bit/Rock Interface (BRI), represented by the pseudo-stiffness K, can be an important factor in determining the effectiveness of the pressure wave generation. It can affect how well the impact energy can be transferred to the diseased vessel, influencing the overall drilling efficiency and penetration rate.

In some embodiments, the pressure wave generation can function to transfer force and energy from the raised features 300 to the plaque following an impact. In some embodiments, the pressure wave generation can be optimized for efficiency with consideration of wave energy radiation into the plaque. In percussive drilling of rock, the impact can be used to generate short-duration compressive loads of magnitudes sufficient to crush the rock. Typically, a hydraulic force accelerating a hammer during tens of milliseconds can be transformed into a compressive force of hundreds of kilonewtons which crushes the rock during hundreds of microseconds. The crushing work has its origin in the kinetic impact energy. Therefore, the efficiency of a percussive drilling process, which can be a key performance parameter, can be commonly defined as the ratio of the crushing work to the impact energy per impact cycle. Other performance parameters, of similar importance, can be associated with, e.g., the energy required to crush a unit volume of rock and the removal of the debris.

In some embodiments, disclosed herein are systems and methods that produce unexpected results clinically related to stenosis. . . . The balloon 100 with strips 200 and raised features 300 can have a consistent percent of stenosis post-treatment. The percent of stenosis post-treatment for the balloon 100 with strips 200 and raised features 300 can be approximately 20%. The percent of stenosis post-treatment for the balloon 100 with strips 200 and raised features 300 can range from 10% to 30%. In some embodiments, the majority of the samples were between 19% and 23%. The percent of stenosis post-treatment for POBA can range from 20% to 50%.

The balloon 100 with strips 200 and raised features 300 can have a consistent percent of stenosis for all pre-treatment reference vessel diameter (RVD). The variation for the balloon 100 with strips 200 and raised features 300 can be less than 20%. The variation for the balloon 100 with strips 200 and raised features 300 can range from about 15% to about 29%. With POBA, the results varied greatly across pre-treatment RVD. The variation for POBA can be greater than 20% The variation for POBA can range from about 20% to 48%. These results were consistent for cases with 99% pre-treatment stenosis. These results were consistent for cases with 100% pre-treatment stenosis.

In some embodiments, disclosed herein are systems and methods that produce unexpected results clinically related to lumen gain. The balloon 100 with strips 200 and raised features 300 can have consistent lumen gain. For all arterial dimensions, the lumen gain was remarkably consistent. The best fit line for serrated balloon 100 can be flat. The balloon 100 with strips 200 and raised features 300 can produce consistent lumen gain. POBA had inconsistent lumen gain. For all arterial dimensions tested, the lumen gain varied greatly. The best fit line for POBA increases lumen gain with arterial dimension. The lumen gain can be greater for larger arteries. The lumen gain varied from 20% to 50%.

The balloon 100 with strips 200 and raised features 300 can produce stable results across all arterial dimensions tested. The balloon 100 with strips 200 and raised features 300 can have the tendency to offer more repeatable lumen gain compared to POBA. The balloon 100 with strips 200 and raised features 300 can have the tendency to offer more repeatable lumen gain independent of the arterial dimension. For POBA, lumen gain appeared to be dependent on the arterial dimension.

For pre-treatment RVD between 1.5 mm and 2 mm, the balloon 100 with strips 200 and raised features 300 can have a percentage stenosis post-treatment of between 10% and 20%. For pre-treatment RVD between 2 mm and 2.5 mm, the balloon 100 with strips 200 and raised features 300 can have a percentage stenosis post-treatment between 10% and 30%. For pre-treatment RVD between 2.5 mm and 3.0 mm, the balloon 100 with strips 200 and raised features 300 can have a percentage stenosis post-treatment between 20% and 30%. For pre-treatment RVD between 3.0 mm and 3.5 mm, the balloon 100 with strips 200 and raised features 300 can have a percentage stenosis post-treatment of between 20% and 30%. For pre-treatment RVD between 3.5 mm and 4.0 mm, the balloon 100 with strips 200 and raised features 300 can have a percentage stenosis post-treatment of between 20% and 30%. The balloon 100 with strips 200 and raised features 300 can produce a consistent percentage stenosis post-treatment compared to POBA.

In some embodiments, disclosed herein are systems and methods that produce unexpected results clinically related to vessel remodeling. Vessel remodeling can include alterations in the structure of the vessel. The balloon 100 with strips 200 and raised features 300 can alter the structure of the vessel wall by creating linear serration lines. The balloon 100 with strips 200 and raised features 300 can change the vessel size at the site of the disease, such as an lesion. The balloon 100 with strips 200 and raised features 300 can increase the cross-sectional dimension or diameter of the vessel. The vessel can positively remodeled by expanding the vessel. The remodeled vessel allows for greater flow therethrough. The balloon 100 with strips 200 and raised features 300 can remodel the vessel such that the vessel maintains the lumen size. The balloon 100 with strips 200 and raised features 300 can facilitate the expanded vessel despite the accumulated lesions.

The balloon 100 with strips 200 and raised features 300 can create linear cracks which can be a structural alternation in the vessel. The balloon 100 with strips 200 and raised features 300 can create a crack past the intima layer. The balloon 100 with strips 200 and raised features 300 can create a linear crack to the medial layer. The balloon 100 with strips 200 and raised features 300 can create changes to the internal elastic lamina. The balloon 100 with strips 200 and raised features 300 can create changes to the intima. The balloon 100 with strips 200 and raised features 300 can create changes to the media.

The balloon 100 with strips 200 and raised features 300 can reverse the progression of cardiovascular conditions. Stenosis can be caused by artherosclerosis whereby the vessel can be narrowed due to deposits. These deposits can be fatty deposits such as plaques which harden over time. The balloon 100 with strips 200 and raised features 300 can reverse this blockage. The balloon 100 with strips 200 and raised features 300 can enlarge the vessel without major disruption the deposits. The balloon 100 with strips 200 and raised features 300 can separate the vessel wall into segments. The adjacent segments of the vessel wall can be separated by cracks that extend to the medial layer. While not bound by a theory, the cracks can allow the segments to more easily expand under pressure. The cracks can allow the segments to expand to allow blood flow to dramatically improve. The cracks can prevent the segments from recoiling to a small diameter.

The balloon 100 with strips 200 and raised features 300 can remodel the vessel. The balloon 100 with strips 200 and raised features 300 can allow the lumen to expand. The lumen gain can be visible by the comparison between the cross-section at each location. For instance, at a first location (designated A for pre-intervention and A′ for post-intervention), the lumen can increase in cross-sectional dimension. The lumen can also be reshaped in some methods. The lumen can change from an oval shape to a more rounded shape. The lumen can change back to a pre-diseased shape. The lumen can change back to a pre-diseased diameter or cross-section.

The balloon 100 with strips 200 and raised features 300 can dramatically remodel the vessel. The balloon 100 with strips 200 and raised features 300 can allow the lumen to expand. The lumen gain can be visible by the comparison between the cross-section at each location. For instance, at a second location (shown as the rightmost location), the lumen can increase in cross-sectional dimension. This lumen gain can be dramatic, such as increasing the ratio of flow post-intervention compared with pre-intervention by 100%, 1000%, 10,000%, 100,000%, or any range of two of the foregoing values. The lumen can also be reshaped in some methods. The lumen can be expanded to accommodate the flow of blood normally accommodated by the vessel. The lumen can be expanded by the fluid flow itself after serration technology, in some methods. The blood flow can be restored through the lumen of the vessel.

The lumen can maintain this expanded shape after the removal of the balloon 100 with strips 200 and raised features 300. The balloon 100 with strips 200 and raised features 300 can be an interventional technology. In some embodiments, the balloon 100 with strips 200 and raised features 300 does not remain in the body of the patient after expansion, in some methods. The balloon 100 with strips 200 and raised features 300 can be removed. In some embodiments, the carrier 1200 with the raised features 300 can remain in the body of the patient after expansion. With POBA, the vessel recoils when the POBA is removed. The vessel recoils to a smaller diameter when the vessel is not supported by the pressure of POBA. The hardened plaque can function to snap the vessel back toward the pre-interventional diameter. With the balloon 100 with strips 200 and raised features 300, the recoil of the vessel can be dramatically less. The vessel does not recoil as much since the vessel can be permanently changed by linear serration lines. The vessel does not recoil because the plaque can be separated into segments. The plaque can be interrupted by the linear serration lines thereby decreasing the ability of the plaque to snap back.

In some embodiments, disclosed herein are systems and methods that produce unexpected results clinically related to stenosis. The balloon 100 with strips 200 and raised features 300 can have a dramatically lower final residual stenosis. The balloon 100 with strips 200 and raised features 300 and POBA were utilized with vessels having a high pre-intervention stenosis percentage. For the samples with the balloon 100 with strips 200 and raised features 300, the average pre-intervention stenosis percentage was 86.2%. For the samples with POBA, the average pre-intervention stenosis percentage was 81.4%. The balloon 100 with strips 200 and raised features 300 had remarkable lumen gain. The balloon 100 with strips 200 and raised features 300 had an average post-intervention stenosis percentage of 17.2%. POBA had an average post-intervention stenosis percentage of 33.7%. POBA had a much smaller lumen gain. The balloon 100 with strips 200 and raised features 300 had a much greater lumen gain.

The balloon 100 with strips 200 and raised features 300 had an improved final stenosis compared with POBA. The final residual stenosis was improved by 49%. The final residual stenosis can be improved over POBA by 10%, at least 10%, 20%, at least 20%, 30%, at least 30%, 40%, at least 40%, 50%, at least 50%, 60%, at least 60%, or any range of two of the foregoing values.

The balloon 100 with strips 200 and raised features 300 can produce stable results across all calcifications tested. The balloon 100 with strips 200 and raised features 300 can have the a tendency to offer more repeatable lumen gain for severely calcified lesions and none/mild/moderate calcified lesions. While not to be limited by a theory, the balloon 100 with strips 200 and raised features 300 can create cracks to the medial layers regardless of the diseased state of the vessel wall. The balloon 100 with strips 200 and raised features 300 can act to segment the vessel wall between the cracks regardless of the diseased state of the vessel wall. These segments can be easier to expand regardless of the diseased state of the vessel wall. The balloon 100 with strips 200 and raised features 300 can have the a tendency to offer more repeatable lumen gain independent of the calcifications. For POBA, lumen gain may be dependent on the calcifications. The calcifications may cause recoil, thus diminishing any lumen gain experienced while the POBA exerted pressure.

The balloon 100 with strips 200 and raised features 300 can have numerous advantages. The strip 200 can increase the column strength of the balloon 100. The strip 200 can be longitudinally placed. The strip 200 can allow force to be transmitted from the proximal end to the distal end of the strip 200. The strip 200 can strengthen the balloon 100 in a longitudinal direction.

In some embodiments, disclosed herein are systems and methods that produce unexpected results clinically related ease of use related to the serration balloon. The strip 200 can facilitate placement of the balloon 100. The strip 200 can stiffen the balloon 100 along the length. The strip 200 can allow the balloon 100 to be pushed without buckling. The strip 200 can be flexible in a direction perpendicular to the longitudinal axis of the strip 200. The strip 200 can allow the balloon 100 to flex right and left as the balloon 100 navigates the vasculature. The strip 200 can allow force to be transmitted from the proximal end to the distal end of the balloon 100.

The strip 200 can improve column strength in the delivery configuration shown in FIG. 58A. The strip 200 can be transversely oriented. The strip 200 can function as a support strut. The strip 200 can have column strength in the folded and pleated configuration. The strip 200 can have column strength when transversely oriented. The strip 200 can have column strength when in the insertion or delivery configuration. The strip 200 can have column strength when folded under the wings 110 of the balloon 100.

The strip 200 can improve column strength any configuration shown in FIGS. 58B-58E. The strip 200 can be less transversely oriented. The strip 200 can be perpendicularly oriented. The strip 200 can have column strength in the expanded configuration. The strip 200 can have column strength when perpendicularly oriented. The strip 200 can have column strength when penetrating the vessel wall. The strip 200 can have column strength when seated in the serrations.

In some embodiments, disclosed herein are systems and methods that produce unexpected results clinically related to dissections. The serrated balloon 100 can greatly reduce the likelihood of dissection. The balloon 100 with strips 200 and raised features 300 can allow the diseased lumen to be expanded and stretched using low pressure. The balloon 100 with strips 200 and raised features 300 can create the serrations, indentations, and/or microperforation at low pressures. The balloon 100 with strips 200 and raised features 300 can create the serrations, indentations, and/or microperforation at pressures between 2 atm and 4 atm. The serrated balloon 100 can facilitate crack propagation at low pressure. The balloon 100 with strips 200 and raised features 300 can facilitate crack propagation at pressures greater than 4 atm. The balloon 100 with strips 200 and raised features 300 can facilitate crack propagation at pressures lower than POBA. The balloon 100 with strips 200 and raised features 300 can create linear serration lines without creating numerous and substantial dissections and elevated flaps. For the majority of interventions at each site, there were no dissections in some methods.

In some embodiments, disclosed herein are systems and methods that produce unexpected results clinically related to the arterial surface. The balloon 100 with strips 200 and raised features 300 can enable the plaque to be dilated more evenly and smoothly. The balloon 100 with strips 200 and raised features 300 can creates segment of the vessel wall which may or may not include plaque. The balloon 100 with strips 200 and raised features 300 can create segments that can easily dilated or pushed outward. The balloon 100 with strips 200 and raised features 300 can create segments that can be expanded independently. The balloon 100 with strips 200 and raised features 300 can create segments that can be expanded with less force. The balloon 100 with strips 200 and raised features 300 can create segments that can be expanded under fluid flow, in some methods. The balloon 100 with strips 200 and raised features 300 can create segments can be dilated with lower pressure. The balloon 100 with strips 200 and raised features 300 can advantageously avoid forming random cracks. The balloon 100 with strips 200 and raised features 300 can create linear serration lines which are predictable. The balloon 100 with strips 200 and raised features 300 can create linear serration lines which are repeatable.

The balloon 100 with strips 200 and raised features 300 can advantageously increase arterial diameter while minimizing vessel injury. The balloon 100 with strips 200 and raised features 300 can advantageously improve the use of angioplasty's mechanism of action. The serrated balloon 100 advantageously can increase volumetric flow. The serrated balloon 100 with strips 200 and raised features 300 can advantageously minimize the degree of dissections and other forms of vessel injury. The serrated balloon 100 with strips 200 and raised features 300 can advantageously reduce the need for stents. The serrated balloon 100 with strips 200 and raised features 300 can advantageously combine angioplasty with longitudinally oriented serrations along the intima of the artery. The serrated balloon 100 with strips 200 and raised features 300 can advantageously alter the mechanism of action of angioplasty alone.

The balloon 100 with strips 200 and raised features 300 can advantageously combine features of angioplasty and serration. The balloon 100 with strips 200 and raised features 300 can advantageously require minimal atmospheric pressure to achieve improvements in blood flow. The balloon 100 can include a set of strips 200 which are integrated into the angioplasty balloon 100. The balloon 100 with strips 200 and raised features 300 can advantageously produce a series of serrated lines along the intima when the balloon 100 is inflated. The strips 200 can penetrate the media. The balloon 100 with strips 200 and raised features 300 can advantageously create serrated lines. The balloon 100 with strips 200 and raised features 300 can advantageously promote the angioplasty energy to follow these lines of weakness which are oriented along the arterial axis. As the lumen expands, the intima and medial tissues can separate more gently and predictably. As the lumen expands, the intima and medial tissues can separate enabling more effective blood flow past the repaired stenotic lesion. The crack can extend past the intimal. The crack can extend into the intimal layers. The crack can penetrate the internal elastic lamina. The crack can extend into the medial layers.

The volume flow rate can be highly dependent of the cross section of the artery. For peripheral arteries, restoring lumen diameter to its unconstrained diameter increases the flow rate. The balloon 100 with strips 200 and raised features 300 can advantageously generate a larger lumen in a consistent and predictable manner. The balloon 100 with strips 200 and raised features 300 can advantageously produce a higher volume flow rate over other forms of angioplasty. The ability for serration angioplasty to achieve higher volume flow rate can be described in relation to vessel remodeling. The balloon 100 with strips 200 and raised features 300 advantageously can treat a diseased vessel such that the vessel behavior, including one or more of compliance, flow dynamics, and internal diameter, can be improved. The balloon 100 with strips 200 and raised features 300 can advantageously allow the internal elastic lamina to be serrated. The balloon 100 with strips 200 and raised features 300 can advantageously allow the internal elastic lamina to relax under the balloon pressure. The balloon 100 with strips 200 and raised features 300 can advantageously allow the treated diseased region to become less turbulent or less rough. The balloon 100 with strips 200 and raised features 300 can advantageously allow the treated diseased region to have fewer interruptions or perturbations in the flow stream. The balloon 100 with strips 200 and raised features 300 can advantageously reduce wall friction. The balloon 100 with strips 200 and raised features 300 can advantageously reduce flow resistance. The balloon 100 with strips 200 and raised features 300 can advantageously allow serration angioplasty to be used as either a standalone treatment. The balloon 100 with strips 200 and raised features 300 can advantageously allow serration angioplasty to be used as a conjunctive therapy. The balloon 100 with strips 200 and raised features 300 can advantageously result in an arterial outline and flow dynamics with minimal to no appearance of vessel or disease discontinuity or disruption to the flow. The balloon 100 with strips 200 and raised features 300 can advantageously allow positive vessel remodeling.

In some embodiments, disclosed herein are systems and methods that produce unexpected results clinically related to volumetric flow rates. The balloon 100 can advantageously improve residual stenosis versus pre-treatment RVD (pre-treatment). The balloon 100 with strips 200 and raised features 300 can advantageously consistently outperform POBA. The balloon 100 with strips 200 and raised features 300 can advantageously achieve at least a two time greater average flow rate ratio over POBA for stenotic lesions. The balloon 100 with strips 200 and raised features 300 can advantageously achieve at least 50% less residual stenosis for stenotic lesions. Other advantages are disclosed herein.

The balloon 100 with strips 200 and raised features 300 can incorporate any technologies of angioplasty balloons catheters described herein. The balloon 100 with strips 200 and raised features 300 can treat patients with peripheral artery disease. The balloon 100 with strips 200 and raised features 300 can replace current treatments. The balloon 100 with strips 200 and raised features 300 can be built on familiar balloon catheter technology. The balloon 100 with strips 200 and raised features 300 can be used in conjunction with current treatments. The balloon 100 with strips 200 and raised features 300 can make subsequent angioplasty treatments more effective. The balloon 100 with strips 200 and raised features 300 can make drug coated balloon treatments more effective. The balloon 100 with strips 200 and raised features 300 can advantageously spare soft tissue. The balloon 100 with strips 200 and raised features 300 can treat severely calcified lesions.

In some configurations, the balloon 100 with strips 200 and raised features 300 advantageously incorporate pressure wave generation technology. The balloon 100 with strips 200 and raised features 300 can utilize pressure waves to break up plaque or other lesions in blocked vessels. The balloon 100 with strips 200 and raised features 300 can utilize lithotripsy. The balloon 100 with strips 200 and raised features 300 can utilize pressure waves to break up deposits. The balloon 100 with strips 200 and raised features 300 can include the generator 800 to provide pressure waves in combination with raised features 300 on the outer surface of the balloon 100. The generator 800 and the raised features 300 can be synergistic. The generator 800 and the raised features 300 can safely modify lesions including deep or highly calcified regions. The generator 800 and the raised features 300 can reliably dilate vessels, even vessels containing highly calcified regions. The generator 800 can produce pressure waves with ultra-sonic vibrations or ultrasound. The generator 800 can reduce cardiovascular calcium deposits. The generator 800 can generate sonic pressure waves. The generator 800 can create pressure waves that produce a localized effect at the treatment site. The generator 800 creates pressure waves that travel through soft vascular tissue. The generator 800 can generate a pressure wave to selectively crack deposits. The generator 800 can generate a pressure wave to crack calcium deposits at the intimal layer. The generator 800 can generate a pressure wave to crack calcium deposits at the medial layer. The generator 800 can generate a pressure wave to crack calcium deposits within the vessel wall. The balloon 100 with strips 200 and raised features 300 can create serrations before use of the generator 800. The balloon 100 with strips 200 and raised features 300 can create serrations after use of the generator 800. The balloon 100 with strips 200 and raised features 300 can be used to dilate the lesion. The balloon 100 with strips 200 and raised features 300 can be used to dilate at low pressure. The balloon 100 with strips 200 and raised features 300 can be used to dilate the vessel for lumen gain. While not bound by a theory, the generator 800 can generate a pressure wave to crack deposits which have a different density than soft tissue.

The generator 800 can create a pressure wave within the fluid of the system 1100. The pressure wave can be transmitted through the liquid medium used to fill the balloon 100. The pressure wave can be transmitted through the wall of the balloon 100. The strips 200 can allow the pressure wave to be transmitted. The pressure wave can pass undisturbed through the balloon 100 with the strips 200 and the raised features 300. The strips 200 and the raised features 300 can include a modified shape to facilitate transmission of the pressure wave. The strips 200 and the raised features 300 can include a modified material to facilitate transmission of the pressure wave. In some embodiments, the material of the strips 200 with the raised features 300 can have a similar density to soft tissue. The strips 200 with the raised features 300 can be modified to enable effective transmission of the pressure wave. The pressure wave may produce an acoustic pulse wave. The pressure wave may produce a high-powered sound wave. The pressure wave may produce sonic pressure waves. The generator 800 can allow fewer strips 200 to be utilized on the balloon 100 while producing similar clinical results. In some embodiments, one or more strips 200 with raised features 300 are utilized.

The generator 800 and the strips 200 can be synergistic. The generator 800 can allow greater spacing between the raised features 300. The generator 800 can allow the raised features 300 to be shallower or shorter in height. The generator 800 can allow the raised features 300 to penetrate through less material. The generator 800 can reduce the layer of deposits thereby decreasing the height to the medial layer. The balloon 100 with strips 200 and raised features 300 can allow the combination of pressure dilation and serrations, with the optional pressure wave generation. The pressure wave generation can be available for all clinical events, but only utilized for highly calcified lesions in some embodiments. The pressure wave generation can be available for all clinical events and utilized for all calcified lesions, in some embodiment. The raised features 300 can reduce the complexity of pressure wave generation by the generator 800. In some embodiments, the generator 800 does not need to reduce all of the deposits. In some embodiments, the generator 800 only needs to reduce the deposits. In some embodiments, the generator 800 could deliver less energy to the treatment site when used with the strip 200 with the raised features.

The combination of the strips 200 and the generator 800 can reduce the thickness of deposits. The generator 800 can deliver less energy when the raised features 300 are used. The raised features 300 can require less penetration when the generator 800 is used. The generator 800 could reduce the thickness of the calcium to a lesser degree than to the medial layer. The raised features 300 could penetrate less thickness due to the pressure disruption of the calcium layer by the generator 800.

The systems and methods can include a design and manufacturing method for one or more strips 200 integrated with the balloon 100. The strips 200 can include raised features 300 as described herein. The balloons 100 with strips 200 and raised features 300 can be configured to create serrations as described herein. The strips 200 can be used in any methods described herein. The strips 200 can be produced by any methods described herein. The strip 200 can have advantages including a lower cost of manufacturing and ease of manufacturing.

The systems described herein can allow for predictable vessel or artery expansion. The systems described herein can produce a serrated line of weakness that guides uniform vessel opening. The serrated line of weakness can be accelerated to crack formation by the generation of pressure waves. The serrated line of weakness can be limited to a longitudinal line along the longitudinal axis of the vessel.

The systems described herein can allow for 360° compliance restoration. The pressure waves and serrations by the raised features 300 can act together to enable predictable luminal gain. The pressure waves and serrations by the raised features 300 can act together to speed up the procedure. The pressure waves and serrations by the raised features 300 can act together to segment the vessel along the circumference, such as in 90 degree segments, 120 degree segments, 180 degree segments, or any range of two of the forgoing values. The pressure waves and serrations by the raised features 300 can act together to restore compliance along the entire circumference of the vessel.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “creating microperforations in an arterial plaque” includes “instructing the creating of microperforations in an arterial plaque.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A system comprising:
a balloon,
a strip comprising raised features, wherein the raised features have a greater length than width, wherein a longitudinal axis of the strip is longitudinally oriented along the balloon, and
a generator configured to propagate a wave through the balloon, wherein the raised features follow the wave that propagates through the balloon, wherein the generator is configured to produce a series of waves, wherein series of waves repeatably drive the raised features in a tapping motion.

2. The system of claim 1, wherein the tapping motion is configured to reduce a time required to yield a hardened diseased tissue.

3. The system of claim 1, wherein the tapping motion is configured to accelerate creation of microperforations in plaque.

4. The system of claim 1, wherein the tapping motion is configured to accelerate creation of a crack along a cleavage plane.

5. The system of claim 1, wherein the tapping motion is configured to reduce a time required to create cracks between microperforations.

6. The system of claim 1, wherein the tapping motion is configured to reduce a time required to break calcium bonds.

7. The system of claim 1, wherein the system returns to a baseline pressure between each wave of the series of waves.

8. The system of claim 1, wherein the wave comprises an ultrasonic pressure wave.

9. The system of claim 1, wherein series of waves repeatably drive lobes of the balloon in a tapping motion.

10. A system comprising:
a balloon,
a strip comprising raised features, wherein the raised features have a greater length than width, wherein a longitudinal axis of the strip is longitudinally oriented along the balloon, and
a generator configured to produce a series of pressure waves, wherein all of the raised features along a length of the balloon move radially outward with the rise in pressure with the balloon, wherein all of the raised features along the length of the balloon move radially inward with the fall in pressure with the balloon.

11. The system of claim 10, wherein proximal raised features move radially outward before middle raised features.

12. The system of claim 11, wherein the middle raised features move radially outward before distal raised features.

13. The system of claim 10, wherein all of the raised features are configured to make contact with a vessel wall simultaneously.

14. The system of claim 10, wherein the system is configured to utilize a hammering motion.

15. The system of claim 15, wherein a frequency of the hammer motion is independent of the speed of the pressure wave.

16. The system of claim 10, wherein the pressure wave is configured to propagate through the balloon to create short bursts of pressure or shock waves including ultrasonic frequencies that enter surrounding tissue.

17. The system of claim 10, wherein the system is configured to generate a serrated line in the tissue in a shorter period of time than when the series of pressure waves is not used.

18. The system of claim 10, wherein each pressure wave in the series of pressure waves is configured to spike to the same maximum pressure.

19. The system of claim 10, wherein a maximum pressure of any pressure wave in the series of pressure waves is between 8 atm and 20 atm.

20. The system of claim 10, wherein any two pressure waves of the series of pressure waves comprises a pressure difference between 4 atm and 20 atm.

21. The system of claim 10, wherein the pressure waves propagate from a source proximal to the proximal end of the balloon.

22. The system of claim 10, wherein the raised features are configured to create cavitation bubbles outside of the balloon.

23. A system comprising:
a balloon,
a strip comprising raised features, wherein the raised features have a greater length than width, wherein a longitudinal axis of the strip is longitudinally oriented along the balloon, and
a generator configured to create a series of impacts between the raised features and diseased tissue via pressure waves delivered to the balloon to accelerate fracturing with each consecutive pressure wave.

24. The system of claim 23, wherein the pressure waves propagate from outside of a body of the balloon.

25. The system of claim 23, wherein the pressure waves propagate from a pressure wave source within a proximal handle of the system.

26. The system of claim 23, wherein the pressure wave transmits a frequency in the ultrasound spectrum.

27. The system of claim 23, wherein the raised features are configured to repeatedly impact without longitudinal movement of the balloon.

28. The system of claim 23, wherein the raised features are configured to create cavitation bubbles.

29. The system of claim 23, wherein the raised features are configured to accelerate fracturing through the internal elastic lamina and into a calcium sheet of the diseased tissue.

30. The system of claim 23, wherein strip and the raised features amplify the mechanical force of the pressure wave by 50 times.
